(19) United States
(12) Patent Application Publication
(10) Pub. No.: US 2021/0317461 A1
(43) Pub. Date: Oct. 14, 2021

(54) OLIGONUCLEOTIDE COMPOSITIONS FOR TARGETING CCR2 AND CSF1R AND USES THEREOF

(71) Applicant: Verseau Therapeutics, Inc., Bedford, MA (US)

(72) Inventors: Tatiana I. Novobrantseva, Wellesley, MA (US); Kevin Kauffman, Somerville, MA (US)

(21) Appl. No.: 17/265,699

(22) PCT Filed: Aug. 9, 2019
(86) PCT No.: PCT/US19/45841

§ 371 Date: Feb. 3, 2021

Related U.S. Application Data
(60) Provisional application No. 62/716,671, filed on Aug. 9, 2018.

(51) Int. Cl. C12N 15/113; A61K 9/51; A61K 45/06; A61K 31/713; A61K 9/00; A61P 35/00

(52) U.S. Cl. CPC C12N 15/1138; A61K 9/5123; A61K 45/06; A61K 31/713; C12N 2320/32; A61P 35/00; C12N 2310/14; C12N 2320/31; A61K 9/0019

(57) ABSTRACT

The present invention is based, in part, on providing oligonucleotide compositions for targeting CCR2, CSF1R, and/or both CCR2 and CSF1R, as well as methods of use thereof, such as to modulate myeloid-derived cell inflammatory phenotypes and immune responses that are mediated by such cells.

### CROSS-REFERENCE TO RELATED APPLICATIONS

This application claims the benefit of U.S. Provisional Application No. 62/716,671 filed on 9 Aug. 2018; the entire contents of said application are incorporated herein in their entirety by this reference.

### BACKGROUND OF THE INVENTION

Myeloid-derived cells, including monocytes and macrophages, are key players of the innate immune system. Circulating monocytes (e.g., monocyte egressing from bone marrow) and tissue resident macrophages migrate to an area in response to environmental signals emanating from the area (e.g., local growth factors, pro-inflammatory cytokines, and microbial compounds) and differentiate into mature/polarized macrophages. Under non-pathological conditions, a balanced population of immune-stimulatory and immune-regulatory macrophages exist in the immune system. In some disease conditions, the balance is interrupted and the imbalance causes many clinical conditions. For example, macrophages infiltrating into a tumor tissue can be switched from being pro-inflammatory to pro-tumorigenic under the influence of tumor cells. It has been shown that certain types of cancers exhibit elevated levels of anti-inflammatory macrophages within the tumor, which are often referred to as tumor associated macrophages (TAMs) or tumor infiltrating macrophages. TAMs in the tumor microenvironment are important regulators of cancer progression and metastasis in both positive and negative ways (Pollard et al. (2004) Nat. Rev. Cancer 4:71-78). The imbalanced polarization of macrophages has been recognized as a key risk factor in many other inflammation related diseases, such as infection, chronic inflammation, inflammatory neurological diseases, cardiovascular diseases, allergy and system autoimmune disorders, multiple sclerosis, rheumatoid arthritis, atherosclerosis, Type I diabetes, Type II diabetes and obesity. Macrophage phenotype is dependent on activation via a classical or an alternative pathway (see, e.g., Classen et al. (2009) Methods Mol. Biol., 531:29-43). Classically activated macrophages are activated by interferon gamma (IFNγ) or lipopolysaccharide (LPS) and display an M1 phenotype. This pro-inflammatory phenotype is associated with increased inflammation and stimulation of the immune system. Alternatively activated macrophages are activated by cytokines like IL-4, IL-10, and IL-13, and display an M2 phenotype. This anti-inflammatory phenotype is associated with decreased immune response, increased wound healing, increased tissue repair, and embryonic development.

Monocytes and macrophages express a variety of surface receptors which can be activated by their corresponding ligands, such as chemokines. The ligand binding activates signaling networks inside the cell to regulate the activation and polarization of monocytes and macrophages. Agents that block the interaction of the ligand-receptor pair in monocytes and macrophages, such as ligand-receptor antagonists, have shown promising therapeutic effects in diseases like cancer. Such agents can modulate the function of myeloid-derived cells, such as the recruitment of monocytes and/or macrophages, the development and polarization/activation of macrophages. For example, in some disease conditions, it is useful to rebalance macrophage populations and/or increase immune-stimulatory macrophage numbers and/or activity.

CCR2 and CSF1R are two surface receptors that are expressed by monocytes and macrophages in response to environmental signals. The activation of CCR2 by its ligand (CCL2) leads to the activation of intracellular signaling cascades that mediate chemotactic response, which induces the recruitment of monocytes and macrophages to the tumor microenvironment. CSF1R blockade using receptor inhibitors can reduce macrophage invasion to local disease sites and can slow disease progression in several disease conditions (Patel et al. (2009) Curr, Top. Med. Chem. 9:599-610). Signaling mediated by CSF1R activation by its ligand (CSF1L) regulates the survival, proliferation, and differentiation of myeloid cells and especially the macrophage lineage. CCR2 antagonists are being investigated as therapeutic agents in cancers and other macrophage-mediated inflammatory diseases, such as rheumatoid arthritis, multiple sclerosis, asthma, and obesity (e.g., Zimmermann et al. (2014) Curr, Top. Med. Chem. 14:1539-1552).

Despite advances in the field of macrophage biology, however, there remains a need for identifying agents that are used alone and in combination to effectively target myeloid-derived cell surface receptors like CCR2 and CSF1R in order to modulate their inflammatory phenotype and use such agents to modulate immune responses.

### SUMMARY OF THE INVENTION

The present invention is drawn, in part, to oligonucleotide compositions for targeting CCR2, CSF1R, or both CCR2 and CSF1R, as well as uses thereof. The compositions encompassed by the present invention provide siRNA molecules that specifically target CCR2 or CSF1R and modulate the activity of myeloid-derived cells. The siRNA molecules have been selected to effectively target CCR2 or CSF1R without off-target effects and to optimize a number of other factors useful for inhibiting these targets. In addition, the present invention also provides formulations comprising such siRNA molecules for enhanced delivery to myeloid-derived cells like monocytes and macrophages. Moreover, without being bound by theory, it is believed that the use of a combination of oligonucleotide compositions described herein and formulations comprising same is particularly effective to inhibit CCR2 and CSF1R activation in order to simultaneously inhibit the trafficking, polarization and activation of monocytes and macrophages in response to an environmental signal, such as a growth factor from tumor cells. Methods for inhibiting CCR2 and CSF1R receptor functions, modulating the recruitment, polarization, and activation of myeloid-derived cells, and treating macrophage-mediated diseases, such as cancer, are also provided.

In one aspect, a composition comprising a) at least one siRNA molecule that hybridizes to a nucleic acid molecule encoding CCR2, b) at least one siRNA molecule that hybridizes to a nucleic acid molecule encoding CSF1R, or c) a combination of a) and b), is provided.

Numerous embodiments are further provided that can be applied to any aspect encompassed by the present invention and/or combined with any other embodiment described herein. For example, in one embodiment, the at least one siRNA molecule that hybridizes to the nucleic acid molecule encoding CCR2 comprises a sense strand having a nucleic acid sequence selected from SEQ ID NO: 6 to SEQ ID NO: 67 and an anti-sense strand having a nucleic acid sequence selected from SEQ ID NO: 68 to SEQ ID NO: 129. In another embodiment, the at least one siRNA molecule that hybridizes to the nucleic acid molecule encoding CSF1R comprises a sense strand having a nucleic acid sequence selected from SEQ ID NO: 130 to SEQ ID NO 248 and an anti-sense strand having a nucleic acid sequence selected from SEQ ID NO: 249 to SEQ ID NO: 367. In still another embodiment, the at least one siRNA molecule that hybridizes to the nucleic acid molecule encoding CCR2 or CSF1R further comprise at least one modification. In yet another embodiment, the modification is a modification to the sugar moiety of the nucleic acid sequence, a nucleobase modification, an internucleoside linker modification, an artificial nucleotide, an end cap modification, or any combinations thereof. In another embodiment, the modification locates in the sense strand of the at least one siRNA molecule. In still another embodiment, the modification locates in the anti-sense strand of the at least one siRNA molecule. In yet another embodiment, the modification locates in the sense and anti-sense strands of the at least one siRNA molecule. In another embodiment, the at least one siRNA molecule that hybridizes to the nucleic acid molecule encoding CCR2 comprises a sense strand having a modified nucleic acid sequence selected from SEQ ID NO: 368 to SEQ ID NO: 486 and SEQ ID NO: 883 to SEQ ID NO: 921, and an anti-sense strand having a modified nucleic acid sequence selected from SEQ ID NO: 487 to SEQ ID NO: 605 and SEQ ID NO: 922 to SEQ ID NO: 960. In still another embodiment, the at least one siRNA molecule that hybridizes to the nucleic acid molecule encoding CCR2 comprises a sense strand having a modified nucleic acid sequence selected from SEQ ID NO: 606 to SEQ ID NO: 743 and SEQ ID NO: 961 to SEQ ID NO: 1001, and an anti-sense strand having a modified nucleic acid sequence selected from SEQ ID NO: 744 to SEQ ID NO: 881 and SEQ ID NO: 1002 to SEQ ID NO: 1042.

In another aspect, a composition comprising a) at least one siRNA duplex that hybridizes to a nucleic acid molecule encoding CCR2, b) at least one siRNA duplex that hybridizes to a nucleic acid molecule encoding CSF1R, or c) a combination of a) and b), wherein the at least one siRNA duplex that hybridizes to the nucleic acid molecule encoding CCR2 comprises a sense strand having a nucleic acid sequence selected from SEQ ID NO: 6 to SEQ ID NO: 67, or a modified nucleic acid sequence selected from SEQ ID NO: 606 to SEQ ID NO: 743, or a modification variant selected from SEQ ID NO: 961 to SEQ ID NO: 1001, and an anti-sense strand having a nucleic acid sequence selected from SEQ ID NO: 68 to SEQ ID NO: 129, or a modified nucleic acid sequence selected from SEQ ID NO: 744 to SEQ ID NO: 881, or a modification variant selected from SEQ ID NO: 1002 to SEQ ID NO: 1042; and/or wherein the at least one siRNA duplex that hybridizes to the nucleic acid molecule encoding CSF1R comprises a sense strand having a nucleic acid sequence selected from SEQ ID NO: 130 to SEQ ID NO: 248, or a modified nucleic acid sequence selected from SEQ ID NO: 368 to SEQ ID NO: 486, or a modification variant selected from SEQ ID NO: 883 to SEQ ID NO: 921, and an anti-sense strand having a nucleic acid sequence selected from SEQ ID NO: 249 to SEQ ID NO: 367, or a modified nucleic acid sequence selected from SEQ ID NO: 487 to SEQ ID NO: 605, or a modification variant selected from SEQ ID NO: 922 to SEQ ID NO: 960, is provided.

As described above, numerous embodiments are further provided that can be applied to any aspect of the present invention and/or combined with any other embodiment described herein. For example, in one embodiment, the at least one siRNA duplex that hybridizes to the nucleic acid molecule encoding CCR2 is duplex XD-09048, XD-09050, XD-09098, XD-09117, XD-09127, XD-09043, XD-09045, XD-09060, XD-09062, XD-09086, XD-09094, XD-09095, XD-09107, XD-09112, XD-09113, XD-09115, XD-09121, XD-09138, XD-09143, or XD-09149, or variants thereof. In another embodiment, the at least one siRNA duplex that hybridizes to the nucleic acid molecule encoding CCR2 is duplex XD-09048, XD-09050, XD-09098, XD-09117 or XD-09127, or variants thereof. In still another embodiment, the at least one siRNA duplex that hybridizes to the nucleic acid molecule encoding CSF1R is duplex XD-08944, XD-08947, XD-08988, XD-08993 or XD-08916, XD-08917, XD-08922, XD-08923, XD-08936, XD-08963, XD-08969, XD-08975, XD-08982, XD-08985, XD-08986, XD-08989, XD-09003, XD-09006, XD-09015, or XD-09021, or variants thereof. In yet another embodiment, the at least one siRNA duplex that hybridizes to the nucleic acid molecule encoding CSF1R is duplex XD-08944, XD-08947, XD-08988, XD-08993 or XD-08916, or variants thereof.

In some embodiments, the composition further comprises a lipid and/or a lipidoid. For example, in one embodiment, the lipidoid is of Formula (VI):

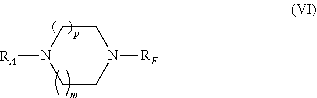

wherein p is an integer between 1 and 3, inclusive; m is an integer between 1 and 3, inclusive; R_{A} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

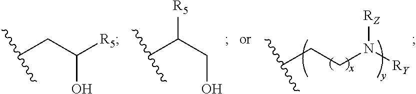

R_{F} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

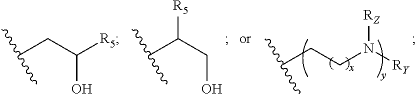

each occurrence of R₅ is independently hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; or substituted or unsubstituted heteroaryl;
wherein, at least one of R_{A}, R_{F}, R_{Y}, and R_{Z} is

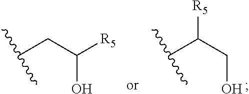

each occurrence of x is an integer between 1 and 10, inclusive; each occurrence of y is an integer between 1 and 10, inclusive; each occurrence of R_{Y} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

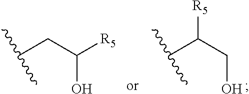

each occurrence of R_{Z} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

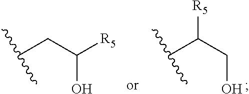

or a pharmaceutically acceptable salt thereof. In another embodiment, p is 1. In still another embodiment, wherein m is 1. In yet another embodiment, each of p and m is 1. In another embodiment, R_{F} is

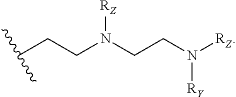

In still another embodiment, R_{A} is

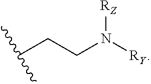

In yet another embodiment, the compound of Formula (VI) is of the formula:

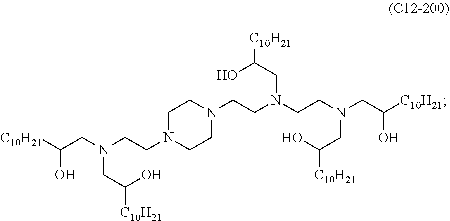

or a salt thereof. In another embodiment, the composition is in the form a lipid nanoparticle. In still another embodiment, the lipid nanoparticle comprises about 1.0% to about 60.0% by mole of C12-200. In yet another embodiment, the lipid nanoparticle further comprises one or more co-lipids. In another embodiment, each co-lipid is selected from disteroylphosphatidyl choline (DSPC), cholesterol, and DMG-PEG. In still another embodiment, the concentration of DSPC is about 1.0% to about 20.0% by mole. In yet another embodiment, the concentration of cholesterol is about 10.0% to about 50.0% by mole. In another embodiment, the concentration of DMG-PEG is about 0.1% to about 5.0% by mole. In still another embodiment, DSPC is present a concentration of about 1.0% to about 20.0% by mole; cholesterol is present at a concentration of about 10.0% to about 50.0% by mole; and DMG-PEG is present a concentration of about 0.1% to about 5.0% by mole. In yet another embodiment, C12-200, DSPC, cholesterol, and DMG-PEG are present at a ratio of 50%:10%:38.5%:1.5%, respectively. In another embodiment, the lipids and lipidoids of the LNP compared to the siRNA molecules are present at a ratio from about 20:1 to about 5:1 by weight. In still another embodiment, the lipids and lipidoids of the LNP compared to the siRNA molecules are present at a ratio of 9:1 by weight. In yet another embodiment, the composition is in a pharmaceutically acceptable formulation.

In still another aspect, a method of generating a myeloid-derived cell having an increased inflammatory phenotype after contact with at least one composition encompassed by the present invention, comprising contacting the myeloid-derived cell with an effective amount of the at least one composition, is provided.

As described above, numerous embodiments are further provided that can be applied to any aspect of the present invention and/or combined with any other embodiment described herein. For example, in one embodiment, the myeloid-derived cell having an increased inflammatory phenotype exhibits one or more of the following after contact with the at least one composition: a) increased expression of cluster of differentiation 80 (CD80), CD86, MHCII, MHCI, interleukin 1-beta (IL-1β), IL-6, CCL3, CCL4, CXCL10, CXCL9, GM-CSF and/or tumor necrosis factor alpha (TNF-α); b) decreased expression of CD206, CD163, CD16, CD53, VSIG4, PSGL-1, TGFb and/or IL-10; c) increased secretion of at least one cytokine or chemokine selected from the group consisting of IL-1β, TNF-α, IL-12, IL-18, GM-CSF, CCL3, CCL4, and IL-23; d) increased ratio of expression of IL-1β, IL-6, and/or TNF-α to expression of IL-10; e) increased CD8+ cytotoxic T cell activation; f) increased recruitment of CD8+ cytotoxic T cell activation; g) increased CD4+ helper T cell activity; h) increased recruitment of CD4+ helper T cell activity; i) increased NK cell activity; j) increased recruitment of NK cell; k) increased neutrophil activity; 1) increased macrophage activity; and/or m) increased spindle-shaped morphology, flatness of appearance, and/or number of dendrites, as assessed by microscopy. In another embodiment, the myeloid-derived cell contacted with the at least one composition are comprised within a population of cells and the at least one composition increases the number of Type 1 and/or M1 macrophages, and/or decreases the number of Type 2 and/or M2 macrophages, in the population of cells. In still another embodiment, the myeloid-derived cell contacted with the at least one composition is comprised within a population of cells and the at least one composition increases the ratio of i) to ii), wherein i) is Type 1 and/or M1 macrophages and ii) is Type 2 and/or M2 macrophages in the population of cells. In yet another embodiment, the myeloid-derived cell is contacted in vitro or ex vivo. In another embodiment, the myeloid-derived cell is a primary myeloid-derived cell. In still another embodiment, the myeloid-derived cell is purified and/or cultured prior to contact with the at least one composition. In yet another embodiment, the myeloid-derived cell is contacted in vivo. In another embodiment, the myeloid-derived cell is contacted in vivo by systemic, peritumoral, or intratumoral administration of the composition. In still another embodiment, the myeloid-derived cell is contacted in a subject in need thereof, optionally wherein the contact is in a tissue microenvironment. In yet another embodiment, the method further comprises contacting the myeloid-derived cell with at least one additional therapeutic agent. In another embodiment, the at least one additional therapeutic agent is an antagonist of CCL2 and/or an antagonist of CSF1. In still another embodiment, the at least one additional therapeutic agent comprises an immunotherapeutic agent that modulates the inflammatory phenotype, optionally wherein the immunotherapeutic agent comprises an immune checkpoint inhibitor, immune-stimulatory agonist, inflammatory agent, cells, a cancer vaccine, and/or a virus.

In yet another aspect, a method of increasing an inflammatory phenotype of myeloid-derived cells in a subject after contact with at least one composition encompassed by the present invention, comprising administering to the subject an effective amount of the at least one composition that contacts the myeloid-derived cells, is provided.

As described above, numerous embodiments are further provided that can be applied to any aspect of the present invention and/or combined with any other embodiment described herein. For example, in one embodiment, the myeloid-derived cells having the increased inflammatory phenotype exhibit one or more of the following after contact with the at least one composition: a) increased expression of cluster of differentiation 80 (CD80), CD86, MHCII, MHCI, interleukin 1-beta (IL-1β), IL-6, CCL3, CCL4, CXCL10, CXCL9, GM-CSF and/or tumor necrosis factor alpha (TNF-α); b) decreased expression of CD206, CD163, CD16, CD53, VSIG4, PSGL-1 and/or IL-10; c) increased secretion of at least one cytokine selected from the group consisting of IL-1β, TNF-α, IL-12, IL-18, and IL-23; d) increased ratio of expression of IL-1β, IL-6, and/or TNF-α to expression of IL-10; e) increased CD8+ cytotoxic T cell activation; f) increased CD4+ helper T cell activity; g) increased NK cell activity; h) increased neutrophil activity; i) increased macrophage activity; and/or j) increased spindle-shaped morphology, flatness of appearance, and/or number of dendrites, as assessed by microscopy. In another embodiment, the at least one composition increases the number of Type 1 and/or M1 macrophages, decreases the number of Type 2 and/or M2 macrophages, and/or increases the ratio of i) to ii), wherein i) is Type 1 and/or M1 macrophages and ii) is Type 2 and/or M2 macrophages, in the subject. In still another embodiment, the number and/or activity of cytotoxic CD8+ T cells in the subject is increased after administration of the at least one composition. In yet another embodiment, the at least one composition is administered systemically, peritumorally, or intratumorally. In another embodiment, the at least one composition contacts the myeloid-derived cells in a tissue microenvironment. In still another embodiment, the method further comprises contacting the myeloid-derived cells with at least one additional therapeutic agent. In yet another embodiment, the at least one additional therapeutic agent is an antagonist of CCL2 and/or an antagonist of CSF1. In another embodiment, the at least one additional therapeutic agent comprises an immunotherapeutic agent that modulates the inflammatory phenotype, optionally wherein the immunotherapeutic agent comprises an immune checkpoint inhibitor, immune-stimulatory agonist, inflammatory agent, cells, a cancer vaccine, and/or a virus. In still another embodiment, the immune checkpoint is selected from the group consisting of PD-1, PD-L1, PD-L2, and CTLA-4. In yet another embodiment, the immune checkpoint is PD-1. In another embodiment, the at least one additional therapeutic agent or regimen is administered before, concurrently with, or after the at least one composition.

In another aspect, a method of sensitizing cancer cells in a subject to cytotoxic CD8+ T cell-mediated killing and/or immune checkpoint therapy comprising administering to the subject a therapeutically effective amount of at least one composition encompassed by the present invention for contacting myeloid-derived cells in the subject, is provided.

As described above, numerous embodiments are further provided that can be applied to any aspect of the present invention and/or combined with any other embodiment described herein. For example, in one embodiment, the at least one composition is administered systemically, peritumorally, or intratumorally. In another embodiment, the method further comprises treating the cancer in the subject by administering to the subject an effective amount of at least one additional therapeutic agent. In still another embodiment, the at least one additional therapeutic agent is an antagonist of CCL2 and/or an antagonist of CSF1. In yet another embodiment, the at least one additional therapeutic agent comprises an immunotherapeutic agent that modulates the inflammatory phenotype of the myeloid-derived cells, optionally wherein the immunotherapeutic agent comprises an immune checkpoint inhibitor, immune-stimulatory agonist, inflammatory agent, cells, a cancer vaccine, and/or a virus. In another embodiment, the immune checkpoint is selected from the group consisting of PD-1, PD-L1, PD-L2, and CTLA-4. In still another embodiment, the immune checkpoint is PD-1. In yet another embodiment, the at least one additional therapeutic agent or regimen is administered before, concurrently with, or after the at least one composition. In another embodiment, the at least one composition reduces the number of proliferating cells in the cancer and/or reduce the volume or size of a tumor comprising the cancer cells. In still another embodiment, the at least one composition increases the amount and/or activity of CD8+ T cells infiltrating a tumor comprising the cancer cells. In yet another embodiment, the at least one composition a) increases the amount and/or activity of M1 macrophages infiltrating a tumor comprising the cancer cells and/or b) decreases the amount and/or activity of M2 macrophages infiltrating a tumor comprising the cancer cells.

In some embodiments, the myeloid-derived cells contacted with the at least one composition have a modulated inflammatory phenotype exhibiting one or more of the following: a) decreased expression of CCR2 and/or CSF1R receptors by monocytes and/or macrophages; b) increased expression of cluster of differentiation 80 (CD80), CD86, MHCII, MHCI, interleukin 1-beta (IL-1β), IL-6, CCL3, CCL4, CXCL10, CXCL9, GM-CSF and/or tumor necrosis factor alpha (TNF-α) by monocytes and/or macrophages; c) decreased expression of CD206, CD163, CD16, CD53, VSIG4, PSGL-1, TGFb and/or IL-10 by monocytes and/or macrophages; d) increased secretion of at least one cytokine or chemokine selected from the group consisting of IL-1β, TNF-α, IL-12, IL-18, GM-CSF, CCL3, CCL4, and IL-2 by monocytes and/or macrophages; e) increased ratio of expression of IL-1β, IL-6, and/or TNF-α to expression of IL-10 by monocytes and/or macrophages; f) increased CD8+ cytotoxic T cell activation; g) increased recruitment of CD8+ cytotoxic T cell activation; h) increased CD4+ helper T cell activity; i) increased recruitment of CD4+ helper T cell activity; j) increased NK cell activity; k) increased recruitment of NK cells; 1) increased neutrophil activity; m) increased macrophage activity; and/or n) increased spindle-shaped morphology, flatness of appearance, and/or number of dendrites, as assessed by microscopy. In one embodiment, the myeloid-derived cell is a macrophage, a monocyte, a circulating bone marrow derived monocyte, a tissue resident macrophage, a macrophage associated with a clinical condition, a Type 1 macrophage, a M1 macrophage, a Type 2 macrophage, a M2 macrophage, a M2c macrophage, a M2d macrophage, and/or a tumor-associated macrophages (TAM). In another embodiment, the cancer is selected from the group consisting of mesothelioma, kidney renal clear cell carcinoma, glioblastoma, lung adenocarcinoma, lung squamous cell carcinoma, pancreatic adenocarcinoma, breast invasive carcinoma, acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous, cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, and uveal melanoma. In still another embodiment, the myeloid-derived cells are comprised within a human tumor model, an animal model of cancer, and/or a thyglycollate peritonitis model. In yet another embodiment, the subject is a mammal. In another embodiment, the mammal is a human, such as a human afflicted with a cancer.

### BRIEF DESCRIPTION OF THE DRAWINGS

FIG. 1A-FIG. 1D show dose response curves for selected oligonucleotide compositions for targeting CSF1R (FIG. 1A) and CCR2 (FIG. 1B), including CSF1R siRNA duplexes and variants (FIG. 1C), and CCR2 siRNA duplexes and variants (FIG. 1D).

FIG. 2 shows the results of silencing both CSF1R and CCR2 using a combination of siRNA duplexes.

FIG. 3A-FIG. 3C show mCSF1R and mCCR2 expression silencing in peritoneal macrophages of mice after intraperitoneal administration of LNPs formulated with mCSF1R and/or mCCR2 siRNAs. FIGS. 3A and 3B show flow cytometry expression plots for n=1 representative mouse per group. FIG. 3C shows normalized mCSF1R and mCCR2 MFI for n=5 mice per group.

FIG. 4A-FIG. 4D show mCSF1R and mCCR2 silencing in blood monocytes of mice after intravenous administration of LNPs formulated with mCSF1R and mCCR2 siRNAs. FIGS. 4A-4C show flow cytometry expression plots for n=1 representative mouse per group. FIG. 4D shows normalized mCSF1R and mCCR2 MFI for n=3 mice per group.

FIG. 5D shows the results of a single-readout in vitro model reporter system using a CSF1R/CCR2 psiCHECK™-2 vector. Renilla/firefly luminesence is plotted normalized to plasmid-transfected untreated cells in the Dual-Glo® Luciferase assay. Data are presented as mean+standard deviation (n=4) and a four-point sigmoidal curve was fit to the data.

### DETAILED DESCRIPTION OF THE INVENTION

The present invention features compositions comprising oligonucleotide compositions that target CCR2 and CSF1R, either alone or in combination, as well as formulations comprising such compositions. Such compositions and formulations can be used in a number of methods, including for modulating myeloid-cell derived cell states, such as converting anti-inflammatory macrophages to pro-inflammatory macrophages in a disease condition or promoting immune responses, such as by increasing CD8+ T cell activity. The compositions and formulations can also be used to modulate immune responses mediated by myeloid-cell derived cells, such as treating cancer by converting pro-tumorigenic macrophages into anti-tumorigenic macrophages.

In particular, the present invention provides small interfering RNA (siRNA) molecules that hybridize to CCR2 and/or CSF1R to antagonize the function of CCR2 and/or CSF1R in myeloid-derived cells, including monocytes and macrophages. Small interfering RNA molecules (also known in the art as “short interfering RNAs”) can induce or mediate RNA interference (RNAi). RNAi is a posttranscriptional process in which small RNA molecules inhibit gene expression by neutralizing targeted mRNA molecules through chromatin remodeling, inhibition of protein translation, or direct mRNA degradation, which can bring about sequence-specific gene silencing. Upon administration, siRNA molecules are recruited to the RNA-induced silencing complex (RISC). This complex is able, via the siRNA molecule, to bind to substantially complementary structures (i.e., the mRNA of a transcribed gene) and degrade them by endonuclease activity. This leads ultimately to inhibition of expression of the corresponding gene that encodes the mRNA complementary to the siRNA molecules (e.g., McManus and Sharp (2002) Nat. Rev, Genet. 3:737-747). Certain siRNA molecules allow for specific on-target silencing of a target gene. Compared with conventional small therapeutic molecules, siRNA molecules offer the advantages of being highly potent and able to act on “non-druggable” targets as they can be designed to affect virtually any gene of interest. Since siRNA molecules do not integrate into the genome and they offer great safety, it is possible to deliver a cocktail of siRNA molecules targeting multiple disease-causing genes in a single delivery system to control complex diseases (e.g., cancer). In accordance with the present invention, a cocktail of siRNA molecules targeting CCR2 and/or CSF1R can be delivered into myeloid-derived cells, including monocytes and macrophages.

### I. Definitions

The term “about,” in some embodiments, encompasses values that are within 1%, 2%, 3%, 4%, 5%, 6%, 7%, 8%, 9%, or 10%, inclusive, or any range in between (e.g., plus or minus 2%-6%), of a value that is measured. In some embodiments, the term “about” refers to the inherent variation of error in a method, assay, or measured value, such as the variation that exists among experiments.

The term “activating receptor” includes immune cell receptors that bind antigen, complexed antigen (e.g., in the context of major histocompatibility complex (MHC) polypeptides), or bind to antibodies. Such activating receptors include T cell receptors (TCR), B cell receptors (BCR), cytokine receptors, LPS receptors, complement receptors, Fc receptors, and other ITAM containing receptors. For example, T cell receptors are present on T cells and are associated with CD3 polypeptides. T cell receptors are stimulated by antigen in the context of MHC polypeptides (as well as by polyclonal T cell activating reagents). T cell activation via the TCR results in numerous changes, e.g., protein phosphorylation, membrane lipid changes, ion fluxes, cyclic nucleotide alterations, RNA transcription changes, protein synthesis changes, and cell volume changes. Similar to T cells activation of macrophages via activation receptors such as, cytokine receptors or pattern associated molecular pattern (PAMP) receptors, results in changes such as protein phosphorylation, alteration to surface receptor phenotype, protein synthesis and release, as well as morphologic changes.

The term “administering” relates to the actual physical introduction of an agent into or onto (as appropriate) a biological target of interest, such as a host and/or subject. A composition can be administered to the cell (e.g., “contacting”) in vitro or in vivo. A composition can be administered to the subject in vivo via an appropriate route of administration. Any and all methods of introducing the composition into the host are contemplated according to the present invention. The method is not dependent on any particular means of introduction and is not to be so construed. Means of introduction are well-known to those skilled in the art, and are also exemplified herein. The term include routes of administration which allow an agent to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which can detrimentally affect its ability to perform its intended function. The agent can be administered alone, or in conjunction with a pharmaceutically acceptable carrier. The agent also can be administered as a prodrug, which is converted to its active form in vivo.

The term “agent” refers to a compound, supramolecular complex, material, and/or combination or mixture thereof. A compound (e.g., a molecule) can be represented by a chemical formula, chemical structure, or sequence. Representative, non-limiting examples of agents, include, e.g., small molecules, polypeptides, proteins, polynucleotides (e.g., RNAi agents, siRNA, miRNA, piRNA, mRNA, antisense polynucleotides, aptamers, and the like), lipids, and polysaccharides. In general, agents can be obtained using any suitable method known in the art. In some embodiments, an agent can be a “therapeutic agent” for use in treating a disease or disorder (e.g., cancer) in a subject (e.g., a human).

The term “agonist” refers to an agent that binds to a target(s) (e.g., a receptor) and activates or increases the biological activity of the target(s). For example, an “agonist” antibody is an antibody that activates or increases the biological activity of the antigen(s) it binds.

The term “antagonist” or “antagonistic” refer to a molecule which is capable of, directly or indirectly, substantially counteracting, reducing or inhibiting the biological activity or activation of a target protein, such as CCR2 and/or CSF1R, as well as isoforms, variants and orthologs thereof. In addition to the siRNA molecules that hybridize to CCR2 or CSF1R, the antagonists can also include monoclonal antibodies, competitive peptides, and small molecules that decrease the activity of CCR2 and/or CSF1R. For example, the CCR2 antagonists can be compounds inhibiting CCR2 signaling and the CSF1R antagonists can be compounds inhibiting CSF1R signaling.

The terms “cancer” or “tumor” or “hyperproliferative” refer to the presence of cells possessing characteristics typical of cancer-causing cells, such as uncontrolled proliferation, immortality, invasive or metastatic potential, rapid growth, and certain characteristic morphological features. In some embodiments, such cells exhibit such characteristics in part or in full due to the expression and activity of immune checkpoint proteins, such as PD-1, PD-L1, PD-L2, and/or CTLA-4.

Cancer cells are often in the form of a tumor, but such cells can exist alone within an animal, or can be a non-tumorigenic cancer cell, such as a leukemia cell. As used herein, the term “cancer” includes premalignant as well as malignant cancers. Cancers include, but are not limited to, a variety of cancers, carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, bone cancer, bone tumors, adult malignant fibrous histiocytoma of bone; childhood, malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytoses such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, bone cancer, brain tumor, lung carcinoma (including lung adenocarcinoma), small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In some embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, Brenner, or undifferentiated.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 60%, 65%, 70%, 75%, 80%, 85%, 90%, 95%, 98%, 99%, 99.5%, 99.9%, or greater of the nucleotide residues of the first portion are capable of base pairing with nucleotide residues in the second portion. More preferably, all nucleotide residues of the first portion are capable of base pairing with nucleotide residues in the second portion. In some embodiments, complementary polynucleotides can be “sufficiently complementary” or can have “sufficient complementarity,” that is, complementarity sufficient to maintain a duplex and/or have a desired activity. For example, in the case of RNAi agents, such complementarity is complementarity between the agent and a target mRNA that is sufficient to partly or completely prevent translation of the mRNA. For example, an siRNA having a “sequence sufficiently complementary to a target mRNA sequence to direct target-specific RNA interference (RNAi)” means that the siRNA has a sequence sufficient to trigger the destruction of the target mRNA by the RNAi machinery or process.

The term “substantially complementary” refers to complementarity in a base-paired, double-stranded region between two nucleic acids and not any single-stranded region such as a terminal overhang or a gap region between two double-stranded regions. The complementarity does not need to be perfect; there can be any number of base pair mismatches. In some embodiments, when two sequences are referred to as “substantially complementary” herein, it is meant that the sequences are sufficiently complementary to each other to hybridize under the selected reaction conditions. Accordingly, substantially complementary sequences can refer to sequences with base-pair complementarity of at least 100, 99, 98, 97, 96, 95, 94, 93, 92, 91, 90, 85, 80, 75, 70, 65, 60 percent or more, or any number in between, in a double-stranded region.

The terms “conjoint therapy” and “combination therapy,” as used herein, refer to the administration of two or more therapeutic agents, e.g., combination of modulators of CCR2 and CSF1R, combination of modulators of CCR2 or CSF1R with at least one additional therapeutic agent, such as an inhibitor of CCL2 or CSF1, combination of modulators of CCR2 and CSF1R further in combination with an addition agent such as an immune checkpoint therapy, and the like. The different agents comprising the combination therapy can be administered concomitant with, prior to, or following, the administration of the other or others. The combination therapy is intended to provide a beneficial (additive or synergistic) effect from the co-action of these therapeutic agents. Administration of these therapeutic agents in combination can be carried out over a defined time period (usually minutes, hours, days, or weeks depending upon the combination selected). In combination therapy, combined therapeutic agent can be applied in a sequential manner, or by substantially simultaneous application.

The term “cytokine” refers to a substance secreted by certain cells of the immune system and has a biological effect on other cells. Cytokines can be a number of different substances such as interferons, interleukins, and growth factors.

The term “gene” encompasses a nucleotide (e.g., DNA) sequence that encodes a molecule (e.g., RNA, protein, etc.) that has a function. A gene generally comprises two complementary nucleotide strands (i.e., dsDNA), a coding strand and a non-coding strand. When referring to DNA transcription, the coding strand is the DNA strand whose base sequence corresponds to the base sequence of the RNA transcript produced (although with thymine replaced by uracil). The coding strand contains codons, while the non-coding strand contains anticodons. During transcription, RNA Pol II binds the non-coding strand, reads the anti-codons, and transcribes their sequence to synthesize an RNA transcript with complementary bases. In some embodiments, the gene sequence (i.e., DNA sequence) listed is the sequence of the coding strand.

The term “gene product” (also referred to herein as “gene expression product” or “expression product”) encompasses products resulting from expression of a gene, such as nucleic acids (e.g., mRNA) transcribed from the gene, and polypeptides or proteins arising from translation of such mRNA. It will be appreciated that certain gene products can undergo processing or modification, e.g., in a cell. For example, mRNA transcripts can be spliced, polyadenylated, etc., prior to translation, and/or polypeptides can undergo co-translational or post-translational processing, such as removal of secretion signal sequences, removal of organelle targeting sequences, or modifications such as phosphorylation, glycosylation, methylation, fatty acylation, etc. The term “gene product” encompasses such processed or modified forms. Genomic mRNA and polypeptide sequences from a variety of species, including human, are known in the art and are available in publicly accessible databases such as those available at the National Center for Biotechnology Information (ncbi.nih.gov) or Universal Protein Resource (uniprot.org). Other databases include, e.g., GenBank, RefSeq, Gene, UniProtKB/SwissProt, UniProtKB/Trembl, and the like. In general, sequences in the NCBI Reference Sequence database can be used as gene product sequences for a gene of interest. It will be appreciated that multiple alleles of a gene can exist among individuals of the same species. Multiple isoforms of certain proteins can exist, e.g., as a result of alternative RNA splicing or editing. In general, where aspects of this disclosure pertain to a gene or gene product, embodiments pertaining to allelic variants or isoforms are encompassed, if applicable, unless indicated otherwise. Certain embodiments can be directed to particular sequence(s), e.g., particular allele(s) or isoform(s).

The term “generating” encompasses any manner in which a desired result is achieved, such as by direct or indirect action. For example, cells having modulated phenotypes described herein can be generated by direct action, such as by contact with at least one agent that modulates one or more biomarkers described herein, and/or by indirect action, such as by propagating cells having a desired physical, genetic, and/or phenotypic attributes.

The terms “high,” “low,” “intermediate,” and “negative” in connection with cellular biomarker expression refers to the amount of the biomarker expressed relative to the cellular expression of the biomarker by one or more reference cells. Biomarker expression can be determined according to any method described herein including, without limitation, an analysis of the cellular level, activity, structure, and the like, of one or more biomarker genomic nucleic acids, ribonucleic acids, and/or polypeptides. In one embodiment, the terms refer to a defined percentage of a population of cells expressing the biomarker at the highest, intermediate, or lowest levels, respectively. Such percentages can be defined as the top 0.1%, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10%, 11%, 12%, 13%, 14%, 15% or more, or any range in between, inclusive, of a population of cells that either highly express or weakly express the biomarker. The term “low” excludes cells that do not detectably express the biomarker, since such cells are “negative” for biomarker expression. The term “intermediate” includes cells that express the biomarker, but at levels lower than the population expressing it at the “high” level. In another embodiment, the terms can also refer to, or in the alternative refer to, cell populations of biomarker expression identified by qualitative or statistical plot regions. For example, cell populations sorted using flow cytometry can be discriminated on the basis of biomarker expression level by identifying distinct plots based on detectable moiety analysis, such as based on mean fluorescence intensities and the like, according to well-known methods in the art. Such plot regions can be refined according to number, shape, overlap, and the like based on well-known methods in the art for the biomarker of interest. In still another embodiment, the terms can also be determined according to the presence or absence of expression for additional biomarkers.

The term “substantially identical” refers to a nucleic acid or amino acid sequence that, when optimally aligned, for example using the methods described below, share at least 60%, 65%, 70%, 75%, 80%, 85%, 90%, 95%, 96%, 97%, 98%, 99%, or 100% sequence identity with a second nucleic acid or amino acid sequence. “Substantial identity” can be used to refer to various types and lengths of sequence, such as full-length sequence, functional domains, coding and/or regulatory sequences, exons, introns, promoters, and genomic sequences. Percent sequence identity between two polypeptides or nucleic acid sequences is determined in various ways that are within the skill in the art, for instance, using publicly available computer software such as BLAST program (Basic Local Alignment Search Tool; (Altschul et al. (1995) J. Mol. Biol. 215:403-410), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. It is understood that for the purposes of determining sequence identity when comparing a DNA sequence to an RNA sequence, a thymine nucleotide is equivalent to a uracil nucleotide. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

The term “immune cell” refers to a cell that is capable of participating, directly or indirectly, in an immune response. Immune cells include, but are not limited to T cells, B cells, antigen presenting cells, dendritic cells, natural killer (NK) cells, natural killer T (NK) cells, lymphokine-activated killer (LAK) cells, monocytes, macrophages, eosinophils, basophils, neutrophils, granulocytes, mast cells, platelets, Langerhan's cells, stem cells, peripheral blood mononuclear cells, cytotoxic T cells, tumor infiltrating lymphocytes (TIL), and the like. An “antigen presenting cell” (APC) is a cell that are capable of activating T cells, and includes, but is not limited to, monocytes/macrophages, B cells and dendritic cells (DCs). The term “dendritic cell” or “DC” refers to any member of a diverse population of morphologically similar cell types found in lymphoid or non-lymphoid tissues. These cells are characterized by their distinctive morphology and high levels of surface MHC-class II expression. DCs can be isolated from a number of tissue sources. DCs have a high capacity for sensitizing MHC-restricted T cells and are very effective at presenting antigens to T cells in situ. The antigens can be self-antigens that are expressed during T cell development and tolerance, and foreign antigens that are present during normal immune processes. The term “neutrophil” generally refers to a white blood cell that makes up part of the innate immune system. Neutrophils typically have segmented nucleic containing about 2-5 lobes. Neutrophils frequently migrate to the site of an injury within minutes following trauma. Neutrophils function by releasing cytotoxic compounds, including oxidants, proteases, and cytokines, at a site of injury or infection. The term “activated DC” is a DC that has been pulsed with an antigen and capable of activating an immune cell. The term “NK cell” has its general meaning in the art and refers to a natural killer (NK) cell. One skilled in the art can easily identify NK cells by determining for instance the expression of specific phenotypic marker (e.g., CD56) and identify its function based on, for example, the ability to express different kind of cytokines or the ability to induce cytotoxicity. The term “B cell” refers to an immune cell derived from the bone marrow and/or spleen. B cells can develop into plasma cells which produce antibodies. The term “T cell” refers to a thymus-derived immune cell that participates in a variety of cell-mediated immune reactions, including CD8+ T cell and CD4+ T cell. Conventional T cells, also known as Tconv or Teffs, have effector functions (e.g., cytokine secretion, cytotoxic activity, anti-self-recognition, and the like) to increase immune responses by virtue of their expression of one or more T cell receptors. Tony or Teffs are generally defined as any T cell population that is not a Treg and include, for example, naïve T cells, activated T cells, memory T cells, resting Tony, or Tony that have differentiated toward, for example, the Th1 or Th2 lineages. In some embodiments, Teffs are a subset of non-regulatory T cells (Tregs). In some embodiments, Teffs are CD4+ Teffs or CD8+ Teffs, such as CD4+ helper T lymphocytes (e.g., Th0, Th1, Tfh, or Th17) and CD8+ cytotoxic T cells (lymphocytes). As described further herein, cytotoxic T cells are CD8+T lymphocytes. “Naïve Tony” are CD4⁺ T cells that have differentiated in bone marrow, and successfully underwent a positive and negative processes of central selection in a thymus, but have not yet been activated by exposure to an antigen. Naïve Tony are commonly characterized by surface expression of L-selectin (CD62L), absence of activation markers such as CD25, CD44 or CD69, and absence of memory markers such as CD45RO. Naïve Tony are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tony are not anergic and can proliferate in response to antigen-based T cell receptor activation (Lechler et al. (2001) Philos. Trans. R. Soc. Lond. Biol. Sci. 356:625-637). In tumors, exhausted cells can present hallmarks of anergy.

The term “immunoregulator” refers to a substance, an agent, a signaling pathway or a component thereof that regulates an immune response. The terms “regulating,” “modifying,” or “modulating” with respect to an immune response refer to any alteration in a cell of the immune system or in the activity of such cell. Such regulation includes stimulation or suppression of the immune system (or a distinct part thereof), which can be manifested by an increase or decrease in the number of various cell types, an increase or decrease in the activity of these cells, or any other changes which can occur within the immune system. Both inhibitory and stimulatory immunoregulators have been identified, some of which can have enhanced function in the cancer microenvironment.

The term “immune response” means a defensive response a body develops against “foreigner” such as bacteria, viruses and substances that appear foreign and harmful. An immune response in particular is the activation and/or action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including antibodies (humoral response), cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues. An anti-cancer immune response refers to an immune surveillance mechanism by which a body recognizes abnormal tumor cells and initiates both the innate and adaptive of the immune system to eliminate dangerous cancer cells.

The innate immune system is a non-specific immune system that comprises the cells (e.g., natural killer cells, mast cells, eosinophils, basophils; and the phagocytic cells including macrophages, neutrophils, and dendritic cells) and mechanisms that defend the host from infection by other organisms. An innate immune response can initiate the productions of cytokines, and active complement cascade and adaptive immune response. The adaptive immune system is specific immune system that is required and involved in highly specialized systemic cell activation and processes, such as antigen presentation by an antigen presenting cell; antigen specific T cell activation and cytotoxic effect.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein.

The term “inhibit” or “downregulate” includes the decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented. Similarly, a biological function, such as the function of a protein, is inhibited if it is decreased as compared to a reference state, such as a control like a wild-type state. Such inhibition or deficiency can be induced, such as by application of an agent at a particular time and/or place, or can be constitutive, such as by a heritable mutation. Such inhibition or deficiency can also be partial or complete (e.g., essentially no measurable activity in comparison to a reference state, such as a control like a wild-type state). Essentially complete inhibition or deficiency is referred to as blocked. The term “promote” or “upregulate” has the opposite meaning.

The term “interaction,” when referring to an interaction between two molecules, refers to the physical contact (e.g., binding) of the molecules with one another. Generally, such an interaction results in an activity (which produces a biological effect) of one or both of said molecules. The activity can be a direct activity of one or both of the molecules, (e.g., signal transduction). Alternatively, one or both molecules in the interaction can be prevented from binding their ligand, and thus be held inactive with respect to ligand binding activity (e.g., binding its ligand and triggering or inhibiting costimulation). To inhibit such an interaction results in the disruption of the activity of one or more molecules involved in the interaction. To enhance such an interaction is to prolong or increase the likelihood of said physical contact, and prolong or increase the likelihood of said activity.

The term “microenvironment” generally refers to the localized area in a tissue area of interest and can, for example, refer to a “tumor microenvironment.” The term “tumor microenvironment” or “TME” refers to the surrounding microenvironment that constantly interacts with tumor cells which is conducive to allow cross-talk between tumor cells and its environment. The tumor microenvironment can include the cellular environment of the tumor, surrounding blood vessels, immune cells, fibroblasts, bone marrow derived inflammatory cells, lymphocytes, signaling molecules and the extracellular matrix. The tumor environment can include tumor cells or malignant cells that are aided and influenced by the tumor microenvironment to ensure growth and survival. The tumor microenvironment can also include tumor-infiltrating immune cells, such as lymphoid and myeloid cells, which can stimulate or inhibit the antitumor immune response, and stromal cells such as tumor-associated fibroblasts and endothelial cells that contribute to the tumor's structural integrity. Stromal cells can include cells that make up tumor-associated blood vessels, such as endothelial cells and pericytes, which are cells that contribute to structural integrity (fibroblasts), as well as tumor-associated macrophages (TAMs) and infiltrating immune cells, including monocytes, neutrophils (PMN), dendritic cells (DCs), T and B cells, mast cells, and natural killer (NK) cells. The stromal cells make up the bulk of tumor cellularity, while the dominating cell type in solid tumors is the macrophage.

The term “modulating” and its grammatical equivalents refer to either increasing or decreasing (e.g., silencing), in other words, either up-regulating or down-regulating.

The term “peripheral blood cell subtypes” refers to cell types normally found in the peripheral blood including, but is not limited to, eosinophils, neutrophils, T cells, monocytes, macrophages, NK cells, granulocytes, and B cells.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “probe” refers to any molecule which is capable of selectively binding to a specifically intended target molecule, for example, a nucleotide transcript or protein encoded by or corresponding to a biomarker nucleic acid. Probes can be either synthesized by one skilled in the art, or derived from appropriate biological preparations. For purposes of detection of the target molecule, probes can be specifically designed to be labeled, as described herein. Examples of molecules that can be utilized as probes include, but are not limited to, RNA, DNA, proteins, antibodies, and organic molecules.

The term “ratio” refers to a relationship between two numbers (e.g., scores, summations, and the like). Although, ratios can be expressed in a particular order (e.g., a to b or a:b), one of ordinary skill in the art will recognize that the underlying relationship between the numbers can be expressed in any order without losing the significance of the underlying relationship, although observation and correlation of trends based on the ratio can be reversed.

The term “receptor” refers to a naturally occurring molecule or complex of molecules that is generally present on the surface of cells of a target organ, tissue or cell type.

The term “cancer response,” “response to immunotherapy,” or “response to modulators of T-cell mediated cytotoxicity/immunotherapy combination therapy” relates to any response of the hyperproliferative disorder (e.g., cancer) to an cancer agent, such as a modulator of T-cell mediated cytotoxicity, and an immunotherapy, preferably to a change in tumor mass and/or volume after initiation of neoadjuvant or adjuvant therapy. Hyperproliferative disorder response can be assessed, for example for efficacy or in a neoadjuvant or adjuvant situation, where the size of a tumor after systemic intervention can be compared to the initial size and dimensions as measured by CT, PET, mammogram, ultrasound or palpation. Responses can also be assessed by caliper measurement or pathological examination of the tumor after biopsy or surgical resection. Response can be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response can be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein can be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality can be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival can be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any cancer therapy. The outcome measurement can be pathologic response to therapy given in the neoadjuvant setting. Alternatively, outcome measures, such as overall survival and disease-free survival can be monitored over a period of time for subjects following cancer therapy for which biomarker measurement values are known. In certain embodiments, the doses administered are standard doses known in the art for cancer therapeutic agents. The period of time for which subjects are monitored can vary. For example, subjects can be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive. The reduction in response can be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to a cancer response, e.g., in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn and Cullen (2002) J. Virol. 76:9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease can be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene encompassed by the present invention, or a fragment thereof, short interfering RNA (siRNA), and small molecules which interfere with or inhibit expression of a target biomarker nucleic acid by RNA interference (RNAi).

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method encompassed by the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the therapies. An increased sensitivity or a reduced sensitivity to a therapeutic treatment is measured according to a known method in the art for the particular treatment and methods described herein below, including, but not limited to, cell proliferative assays (Tanigawa et al. (1982) Cancer Res. 42:2159-2164) and cell death assays (Weisenthal et al. (1984) Cancer Res. 94:161-173; Weisenthal et al. (1985) Cancer Treat Rep. 69:615-632; Weisenthal et al., In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993:415-432; Weisenthal (1994) Contrib. Gynecol. Obstet. 19:82-90). The sensitivity or resistance can also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

“Short interfering RNA” (siRNA), also referred to herein as “small interfering RNA” is defined as an agent which functions to inhibit expression of a target biomarker nucleic acid, e.g., by RNAi. An siRNA can be chemically synthesized, can be produced by in vitro transcription, or can be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and can contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 17-29 nucleotide, 19-25 nucleotide, etc. region) antisense strand, followed by a 4-10 nucleotide loop (e.g., a 4, 5, 6, 7, 8, 9, or 10 base linker region), and the analogous sense strand. Alternatively, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA 9:493-501).

RNA interfering agents, e.g., siRNA molecules, can be administered to a patient having or at risk for having cancer, to inhibit expression of a biomarker gene which is overexpressed in cancer and thereby treat, prevent, or inhibit cancer in the subject.

The term “selective modulator” or “selectively modulate” as applied to a biologically active agent refers to the agent's ability to modulate the target, such as a cell population, signaling activity, etc. as compared to off-target cell population, signaling activity, etc. via direct or interact interaction with the target. For example, an agent that selectively inhibits the interaction between a protein and one natural binding partner over another interaction between the protein and another binding partner, and/or such interaction(s) on a cell population of interest, inhibits the interaction at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 2× (times), 3×, 4×, 5×, 6×, 7×, 8×, 9×, 10×, 15×, 20×, 25×, 30×, 35×, 40×, 45×, 50×, 55×, 60×, 65×, 70×, 75×, 80×, 85×, 90×, 95×, 100×, 105×, 110×, 120×, 125×, 150×, 200×, 250×, 300×, 350×, 400×, 450×, 500×, 600×, 700×, 800×, 900×, 1000×, 1500×, 2000×, 2500×, 3000×, 3500×, 4000×, 4500×, 5000×, 5500×, 6000×, 6500×, 7000×, 7500×, 8000×, 8500×, 9000×, 9500×, 10000×, or greater, or any range in between, inclusive, against at least one other binding partner. Such metrics are typically expressed in terms of relative amounts of agent required to reduce the interaction/activity by half. Such metrics apply to any other selectivity arrangement, such as binding of a nucleic acid molecule to one or more target sequences.

More generally, the term “selective” refers to a preferential action or function. The term “selective” can be quantified in terms of the preferential effect in a particular target of interest relative to other targets. For example, a measured variable (e.g., modulation of biomarker expression in desired cells versus other cells, the enrichment and/or deletion of desired cells versus other cells, etc.) can be 10%, 20%, 25%, 30%, 35%, 40%, 45%, 50%, 55%, 60%, 65%, 70%, 75%, 80%, 85%, 90%, 95%, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 70-fold, 80-fold, 90-fold, 100-fold, or greater or any range in between inclusive (e.g., 50% to 16-fold), different in a target of interest versus unintended or undesired targets. The same fold analysis can be used to confirm the magnitude of an effect in a given tissue, cell population, measured variable, and/or measured effect, and the like, such as cell ratios, hyperproliferative cell growth rate or volume, cell proliferation rate, etc. cell numbers, and the like.

By contrast, the term “specific” refers to an exclusionary action or function. For example, specific modulation of an interaction between a protein and one binding partner refers to the exclusive modulation of that interaction and not to any significant modulation of the interaction between the protein and another binding partner. In another example, specific binding of an antibody to a predetermined antigen refers to the ability of the antibody to bind to the antigen of interest without binding to other antigens. Typically, the antibody binds with an affinity (K_{D}) of approximately less than 1×10⁻⁷M, such as approximately less than 10⁻⁸M, 10⁻⁹ M, 10⁻¹⁰ M, 10⁻¹¹M, or even lower when determined by surface plasmon resonance (SPR) technology in a BIACORE® assay instrument using an antigen of interest as the analyte and the antibody as the ligand, and binds to the predetermined antigen with an affinity that is at least 1.1, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. In addition, K_{D} is the inverse of KA. The phrases “an antibody recognizing an antigen” and “an antibody specific for an antigen” are used interchangeably herein with the term “an antibody which binds specifically to an antigen.”

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. The term is intended to encompass all stereoisomers, geometric isomers, tautomers, and isotopes of a chemical structure of interest, unless otherwise indicated.

The term “subject” refers to an animal, vertebrate, mammal, or human, especially one to whom an agent is administered, e.g., for experimental, diagnostic, and/or therapeutic purposes, or from whom a sample is obtained or on whom a procedure is performed. In some embodiments, a subject is a mammal, e.g., a human, non-human primate, rodent (e.g., mouse or rat), domesticated animals (e.g., cows, sheep, cats, dogs, and horses), or other animals, such as llamas and camels. In some embodiments, the subject is human. In some embodiments, the subject is a human subject with a cancer. The term “subject” is interchangeable with “patient.”

The term “survival” includes all of the following: survival until mortality, also known as overall survival (wherein said mortality can be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival can be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

The term “synergistic effect” refers to the combined effect of two or more cancer agents (e.g., a modulator of biomarkers listed in Table 1 and/or Table 2 and immunotherapy combination therapy) can be greater than the sum of the separate effects of the cancer agents/therapies alone.

The term “target” refers to a gene or gene product that is modulated, inhibited, or silenced by an agent, composition, and/or formulation described herein. A target gene or gene product includes wild-type and mutant forms. Non-limiting, representative lists of targets encompassed by the present invention are provided in Table 1 and Table 2. Similarly, the term “target”, “targets”, or “targeting” used as a verb refers to modulating the activity of a target gene or gene product. Targeting can refer to upregulating or downregulating the activity of a target gene or gene product.

The term “therapeutic effect” encompasses a local or systemic effect in animals, particularly mammals, and more particularly humans, caused by a pharmacologically active substance. The term thus means any substance intended for use in the diagnosis, cure, mitigation, treatment, or prevention of disease or in the enhancement of desirable physical or mental development and conditions in an animal or human. A prophylactic effect encompassed by the term encompasses delaying or eliminating the appearance of a disease or condition, delaying or eliminating the onset of symptoms of a disease or condition, slowing, halting, or reversing the progression of a disease or condition, or any combination thereof.

The term “effective amount” or “effective dose” of an agent (including a composition and/or formulation comprising such an agent) refers to the amount sufficient to achieve a desired biological and/or pharmacological effect, e.g., when delivered to a cell or organism according to a selected administration form, route, and/or schedule. As will be appreciated by those of ordinary skill in this art, the absolute amount of a particular agent or composition that is effective can vary depending on such factors as the desired biological or pharmacological endpoint, the agent to be delivered, the target tissue, etc. Those of ordinary skill in the art will further understand that an “effective amount” can be contacted with cells or administered to a subject in a single dose, or through use of multiple doses, in various embodiments. The term “effective amount” can be a “therapeutically effective amount.”

The terms “therapeutically effective amount” refers to that amount of an agent that is effective for producing some desired therapeutic effect in at least a sub-population of cells in an animal at a reasonable benefit/risk ratio applicable to any medical treatment. Toxicity and therapeutic efficacy of subject compounds can be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, Similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

The term “tolerance” or “unresponsiveness” includes refractivity of cells, such as immune cells, to stimulation, e.g., stimulation via an activating receptor or a cytokine. Unresponsiveness can occur, e.g., because of exposure to immunosuppressants or exposure to high doses of antigen. Several independent methods can induce tolerance. One mechanism is referred to as “anergy,” which is defined as a state where cells persist in vivo as unresponsive cells rather than differentiating into cells having effector functions. Such refractivity is generally antigen-specific and persists after exposure to the tolerizing antigen has ceased. For example, anergy in T cells is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134). Another mechanism is referred to as “exhaustion.” T cell exhaustion is a state of T cell dysfunction that arises during many chronic infections and cancer. It is defined by poor effector function, sustained expression of inhibitory receptors and a transcriptional state distinct from that of functional effector or memory T cells.

A “transcribed polynucleotide” or “nucleotide transcript” is a polynucleotide (e.g., an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g., splicing), if any, of the RNA transcript, and reverse transcription of the RNA transcript.

The term “treat” refers to the therapeutic management or improvement of a condition (e.g., a disease or disorder) of interest. Treatment can include, but is not limited to, administering an agent or composition (e.g., a pharmaceutical composition) to a subject. Treatment is typically undertaken in an effort to alter the course of a disease (which term is used to indicate any disease, disorder, syndrome or undesirable condition warranting or potentially warranting therapy) in a manner beneficial to the subject. The effect of treatment can include reversing, alleviating, reducing severity of, delaying the onset of, curing, inhibiting the progression of, and/or reducing the likelihood of occurrence or recurrence of the disease or one or more symptoms or manifestations of the disease. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. A therapeutic agent can be administered to a subject who has a disease or is at increased risk of developing a disease relative to a member of the general population. In some embodiments, a therapeutic agent can be administered to a subject who has had a disease but no longer shows evidence of the disease. The agent can be administered e.g., to reduce the likelihood of recurrence of evident disease. A therapeutic agent can be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur or to reduce the severity of the disease should it occur. The subject can have been identified as being at risk of developing the disease (e.g., at increased risk relative to the general population or as having a risk factor that increases the likelihood of developing the disease.

The term “unresponsiveness” includes refractivity of cancer cells to therapy or refractivity of therapeutic cells, such as immune cells, to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134).

The term “vaccine” refers to a composition for generating immunity for the prophylaxis and/or treatment of diseases.

### II. Myeloid-Derived Cells, CCR2, and CSF1R

### Myeloid-Derived Cells

Hematopoietic stem cells (HSCs) give rise to committed lymphoid or myeloid progenitor cells. Myeloid progenitor cells in turn give rise to myeloid-derived cells, which include monocytes,

myeloid dendritic, myeloid erythroid, erythroid, megakaryocytes, granulocyte/macrophage, granulocyte, and macrophage cells. The term “myeloid-derived cells” can refer to a granulocyte or monocyte precursor cell in bone marrow or spinal cord, or a resemblance to those found in the bone marrow or spinal cord. The myeloid cell lineage includes circulating monocytic cells in the peripheral blood and the cell populations that they become following maturation, differentiation, and/or activation. These populations include non-terminally differentiated myeloid cells, myeloid derived suppressor cells, and differentiated macrophages. Differentiated macrophages include non-polarized and polarized macrophages, resting and activated macrophages. Without being limiting, the myeloid lineage can also include granulocytic precursors, polymorphonuclear derived suppressor cells, differentiated polymorphonuclear white blood cells, neutrophils, granulocytes, basophils, eosinophils, monocytes, macrophages, microglia, myeloid derived suppressor cells, dendritic cells and erythrocytes.

The term “committed myeloid progenitor cells” refers to cell populations capable of differentiating into any of the terminally differentiated cells of the myeloid lineage. Encompassed within the myeloid progenitor cells are the common myeloid progenitor cells (CMP), a cell population characterized by limited or non-self-renewal capacity but which is capable of cell division to form granulocyte/macrophage progenitor cells (GMP) and megakaryocyte/erythroid progenitor cells (MEP). The marker phenotypes useful for identifying CMPs include those commonly known in the art. For CMP cells of murine origin, the cell population is characterized by the marker phenotype c-Kit^{high} (CD117) CD16^{low} CD34^{low} Sca-1^{neg}Lin^{neg} and further characterized by the marker phenotypes FcyR^{lo} IL-7Rα^{neg} (CD127). The murine CMP cell population is also characterized by the absence of expression of markers that include B220, CD4, CD8, CD3, Ter119, Gr-1 and Mac-1. For CMP cells of human origin, the cell population is characterized by CD34⁺CD38⁺ and further characterized by the marker phenotypes CD123⁺ (IL-3Rα) CD45RA^{neg}. The human CMP cell population is also characterized by the absence of cell markers CD3, CD4, CD7, CD8, CD10, CD11b, CD14, CD19, CD20, CD56, and CD234a. Descriptions of marker phenotypes for various myeloid progenitor cells are described in, for example, U.S. Pat. Nos. 6,465,247 and 6,761,883.

Granulocyte/macrophage progenitor cell (GMP). The cells of this progenitor cell population are characterized by their capacity to give rise to granulocytes (e.g., basophils, eosinophils, and neutrophils) and macrophages. Similar to other committed progenitor cells, GMPs lack self-renewal capacity. Murine GMPs are characterized by the marker phenotype c-Kit^{hi} (CD117) Sca-1^{neg}Fc□R^{hi} (CD16) IL-7Rγ^{neg}CD34P^{pos}. Murine GMPs also lack expression of markers B220, CD4, CD8, CD3, Gr-1, Mac-1, and CD90. Human GMPs are characterized by the marker phenotype CD34⁺CD38⁺CD123+CD45RA⁺. Human GMP cell populations are also characterized by the absence of markers CD3, CD4, CD7, CD8, CD10, CD11b, CD14, CD19, CD20, CD56, and CD235a.

Megakaryocyte/erythroid progenitor cells (MEP), which are derived from the CMPs, are characterized by their capability of differentiating into committed megakaryocyte progenitor and erythroid progenitor cells. Mature megakaryocytes are polyploid cells that are precursors for formation of platelets, a developmental process regulated by thrombopoietin. Erythroid cells are formed from the committed erythroid progenitor cells through a process regulated by erythropoietin, and ultimately differentiate into mature red blood cells. Murine MEPs are characterized by cell marker phenotype c-Kit^{hi} and IL-7Ra^{neg} and further characterized by marker phenotypes FcyR^{lo} and CD34^{low}. Murine MEP cell populations are also characterized by the absence of markers B220, CD4, CD8, CD3, Gr-1, and CD90. Another exemplary marker phenotype for mouse MEPs is c-kit^{high}Sca-1^{neg}Lin^{neg/low}CD16^{low}CD34^{low}. Human MEPs are characterized by marker phenotypes CD34⁺CD38⁺CD123^{neg}CD45RA^{neg}. Human MEP cell populations are also characterized by the absence of markers CD3, CD4, CD7, CD8, CD10, CD11b, CD14, CD19, CD20, CD56, and CD235a.

Further restricted progenitor cells in the myeloid lineage are the granulocyte progenitor, macrophage progenitor, megakaryocyte progenitor, and erythroid progenitor. Granulocyte progenitor cells are characterized by their capability to differentiate into terminally differentiated granulocytes, including eosinophils, basophils, neutrophils. The GPs typically do not differentiate into other cells of the myeloid lineage. With regards to the megakaryocyte progenitor cell (MKP), these cells are characterized by their capability to differentiate into terminally differentiated megakaryocytes but generally not other cells of the myeloid lineage (see, e.g., PCT Publ. No. WO 2004/024875).

### Monocytes and Macrophages

In some embodiments, the myeloid-derived cells of interest are monocytes and/or macrophages.

The term “monocytes” refers to a leukocyte that can differentiate into macrophages and myeloid dendritic cells. Monocytes are found among peripheral blood mononuclear cells (PBMCs), which also comprise other hematopoietic and immune cells, such as B cells, T cells, NK cells, and the like. Monocytes are produced by the bone marrow from hematopoietic stem cell precursors called monoblasts. Monocytes have two main functions in the immune system: (1) they can exit the bloodstream to replenish resident macrophages and dendritic cells (DCs) under normal states, and (2) they can quickly migrate to sites of infection in the tissues and divide/differentiate into macrophages and inflammatory dendritic cells to elicit an immune response in response to inflammation signals. Monocytes are usually identified in stained smears by their large bilobate nucleus. Monocytes also express chemokine receptors and pathogen recognition receptors that mediate migration from blood to tissues during infection. They produce inflammatory cytokines and phagocytose cells. There are at least three types of monocytes in humans, including 1) classical monocytes, which are characterized by high level expression of CD14 cell surface receptor (CD14⁺⁺CD16⁻ monocytes), 2) non-classical monocytes, which are characterized by low level expression of CD14 and additional co-expression of the CD16 receptor (CD14⁺CD16⁺⁺ monocyte), and 3) intermediate monocytes, which are characterized by high level expression of CD14 and low level expression of CD16 (CD14⁺⁺CD16⁺ monocytes).

Macrophages are critical immune effectors and regulators of inflammation and the innate immune response. Macrophages are heterogeneous, tissue-resident, terminally-differentiated, innate myeloid cells, which have remarkable plasticity and can change their physiology in response to local cues from the microenvironment and can assume a spectrum of functional requirements from host defense to tissue homeostasis (Ginhoux et al. (2016) Nat. Immunol. 17:34-40). Macrophages are present in virtually all tissues in the body. They are either tissue resident macrophages, for example Kupffer cells that reside in liver, or derived from circulating monocytic precursors (i.e., monocytes) which mainly originate from bone marrow and spleen reservoirs and migrate into tissue in the steady state or in response to inflammation or other stimulating cues. For example, monocytes can be recruited from the blood to tissue to replenish tissue specific macrophages of the bone, alveoli (lung), central nervous system, connective tissues, gastrointestinal tract, live, spleen and peritoneum.

The term “tissue-resident macrophages” refers to a heterogeneous populations of immune cells that fulfill tissue-specific and/or micro-anatomical niche-specific functions such as tissue immune-surveillance, response to infection and the resolution of inflammation, and dedicated homeostatic functions. Tissue resident macrophages originate in the yolk sac of the embryo and mature in one particular tissue in the developing fetus, where they acquire tissue-specific roles and change their gene expression profile Local proliferation of tissue resident macrophages, which maintain colony-forming capacity, can directly give rise to populations of mature macrophages in the tissue. Tissue resident macrophages can also be identified and named according to the tissues they occupy. For example, adipose tissue macrophages occupy adipose tissue, Kupffer cells occupy liver tissue, sinus histiocytes occupy lymph nodes, alveolar macrophages (dust cells) occupy pulmonary alveoli, Langerhans cells occupy skin and mucosal tissue, histiocytes leading to giant cells occupy connective tissue, microglia occupy central nervous system (CNS) tissue, Hofbauer cells occupy placental tissue, intraglomerular mesangial cells occupy kidney tissue, osteoclasts occupy bone tissue, epithelioid cells occupy granulomas, red pulp macrophages (sinusoidal lining cells) occupy the red pulp of spleen tissue, peritoneal cavity macrophages occupy peritoneal cavity tissue, lysomac cells occupy Peyer's patch tissue, and pancreatic macrophages occupy pancreatic tissue.

Macrophages, in addition to host defense against infectious agents and other inflammation reaction, can perform different homeostatic functions, including but not limited to, development, wound healing and tissue repairing, and regulation of immune response. Macrophages, first recognized as phagocytosis cells in the body which defend infections through phagocytosis, are essential components of innate immunity. In response to pathogens and other inflammation stimuli, activated macrophages can engulf infected bacteria and other microbes; stimulate inflammation and release a cocktail of pro-inflammatory molecules to these intracellular microorganisms. After engulfing the pathogens, macrophages present pathogenic antigens to T cells to further activate adaptive immune response for defense. Exemplary pro-inflammatory molecules include cytokines IL-1β, IL-6 and TNF-α, chemokine MCP-1, CXC-5 and CXC-6, and CD40L.

In addition to their contribution to host defense against infections, macrophages play vital homeostatic roles, independent of their involvement in immune responses. Macrophages are prodigious phagocytic cells that clear erythrocytes and the released substances such as iron and hemoglobin can be recycled for the host to reuse. This clearance process is a vital metabolic contribution without which the host would not survive.

Macrophages are also involved in the removal of cellular debris that is generated during tissue remodeling, and rapidly and efficiently clear cells that have undergone apoptosis. Macrophages are believed to be involved in steady-state tissue homeostasis via the clearance of apoptotic cells. These homeostatic clearance processes are generally mediated by surface receptors on macrophages including scavenger receptors, phosphatidyl serine receptors, the thrombospondin receptor, integrins and complement receptors. These receptors that mediate phagocytosis either fail to transduce signals that induce cytokine-gene transcription or actively produce inhibitory signals and/or cytokines. The homeostatic function of macrophages is independent of other immune cells.

Macrophages can also clear cellular debris/necrotic cells that results from trauma or other damages to cells. Macrophages detect the endogenous danger signals that are present in the debris of necrotic cells through toll-like receptors (TLRs), intracellular pattern-recognition receptors and the interleukin-1 receptor (IL-1R), most of which signal through the adaptor molecule myeloid differentiation primary-response gene 88 (MyD88). The clearance of cellular debris can markedly alter the physiology of macrophages. Macrophages that clear necrosis can undergo dramatic changes in their physiology, including alterations in the expression of surface proteins and the production of cytokines and pro-inflammatory mediators. The alterations in macrophage surface-protein expression in response to these stimuli could potentially be used to identify biochemical markers that are unique to these altered cells.

Macrophages have important functions in maintaining homeostasis in many tissues such as white adipose tissue, brown adipose tissue, liver and pancreas. Tissue macrophages can quickly respond to changing conditions in a tissue, by releasing cell signaling molecules that trigger a cascade of changes allowing tissue cells to adapt. For instance, macrophages in adipose tissue regulate the production of new fat cells in response to changes in diet (e.g., macrophages in white adipose tissue) or exposure to cold temperatures (e.g., macrophages in brown adipose tissue). Macrophages in the liver, known as Kupffer cells, regulate the breakdown of glucose and lipids in response to dietary changes. Macrophages in pancreas can regulate insulin production in response to high fat diet.

Macrophages can also contribute to wound healing and tissue repair. For example, macrophages, in response to signals derived from injured tissues and cells, can be activated and induce a tissue-repair response to repair damaged tissue (Minutti et al. (2017) Science 356:1076-1080).

During embryonic development, macrophages also play a key role in tissue remodeling and organ development. For example, resident macrophages actively shape the development of blood vessels in neonatal mouse hearts (Leid et al. (2016) Circ. Res. 118:1498-1511). Microglia in the brain can produce growth factors that guide neurons and blood vessels in developing brain during embryonic development. Similarly, CD95L, a macrophage-produced protein, binds to CD95 receptors on the surface of neurons and developing blood vessels in the brains of mouse embryos and increases neuron and blood vessel development (Chen et al. (2017) Cell Rep. 19:1378-1393). Without the ligand, neurons branch less frequently, and the resulting adult brain exhibits less electrical activity Monocyte-derived cells known as osteoclasts are involved in bone development, and mice that lack these cells develop dense, hardened bones—a rare condition known as osteopetrosis. Macrophages also orchestrate development of the mammary gland and assist in retinal development in the early postnatal period (Wynn et al. (2013) Nature 496:445-455).

As described above, macrophages regulate immune systems. In addition to the presentation of antigens to T cells, macrophages can provide immunosuppressive/inhibitory signals to immune cells in some conditions. For example, in the testis, macrophages help create a protective environment for sperm from being attacked by the immune system. Tissue resident macrophages in the testis produce immunosuppressant molecules that prevent immune cell reaction against sperm (Mossadegh-Keller et al. (2017) J. Exp. Med. 214:10.1084/jem.20170829).

The plasticity of macrophages in response to different environment signals and in agreement with their functional requirements has resulted in a spectrum of macrophage activation states, including two extremes of the continuum, namely “classically activated” M1 and “alternatively activated” M2 macrophages.

The term “activation” refers to the state of a monocyte and/or macrophage that has been sufficiently stimulated to induce detectable cellular proliferation and/or has been stimulated to exert its effector function, such as induced cytokine expression and secretion, phagocytosis, cell signaling, antigen processing and presentation, target cell killing, and pro-inflammatory function.

The term “M1 macrophages” or “classically activated macrophages” refers to macrophages having a pro-inflammatory phenotype. The term “macrophage activation” (also referred to as “classical activation”) was introduced by Mackaness in the 1960s in an infection context to describe the antigen-dependent, but non-specific enhanced, microbicidal activity of macrophages toward BCG (bacillus Calmette-Guerin) and Listeria upon secondary exposure to the pathogens (Mackaness (1962) J. Exp. Med. 116:381-406). The enhancement was later linked with Th1 responses and IFN-γ production by antigen-activated immune cells (Nathan et al. (1983) J. Exp. Med. 158:670-689) and extended to cytotoxic and antitumoral properties (Pace et al. (1983) Proc. Natl. Acad. Sci. U.S.A. 80:3782-3786; Celada et al. (1984) J. Exp. Med. 160:55-74). Therefore, any macrophage functionality that enhances inflammation by cytokine secretion, antigen presentation, phagocytosis, cell-cell interactions, migration, etc. is considered pro-inflammatory. In vitro and in vivo assays can measure different endpoints: general in vitro measurements include pro-inflammatory cell stimulation as measured by proliferation, migration, pro-inflammatory Th1 cytokine/chemokine secretion and/or migration, while general in vivo measurements further include analyzing pathogen fighting, tissue injury immediate responders, other cell activators, migration inducers, etc. For both in vitro and in vivo, pro-inflammatory antigen presentation can be assessed. Bacterial moieties, such as lipopolysaccharide (LPS), certain Toll-like receptor (TLR) agonists, the Th1 cytokine interferon-gamma (IFNγ) (e.g., IFNγ produced by NK cells in response to stress and infections, and T helper cells with sustained production) and TNF polarize macrophages along the M1 pathway. Activated M1 macrophages phagocytose and destroy microbes, eliminate damaged cells (e.g., tumor cells and apoptotic cells), present antigen to T cells for increasing adaptive immune responses, and produce high levels of pro-inflammatory cytokines (e.g., IL-1, IL-6, and IL-23), reactive oxygen species (ROS), and nitric oxide (NO), as well as activate other immune and non-immune cells. Characterized by their expression of inducible nitric oxide synthase (iNOS), reactive oxygen species (ROS), and production of the Th1-associated cytokine, IL-12, M1 macrophages are well-adapted to promote a strong immune response. The metabolism of M1 macrophages is characterized by enhanced aerobic glycolysis, converting glucose into lactate, increased flux through the pentose phosphate pathway (PPP), fatty acid synthesis, and a truncated tricarboxylic acid (TCA) cycle, leading to accumulation of succinate and citrate.

A “Type 1” or “M1-like” monocyte and/or macrophage is a monocyte and/or macrophage capable of contributing to a pro-inflammatory response that is characterized by at least one of the following: producing inflammatory stimuli by secreting at least one pro-inflammatory cytokine, expressing at least one cell surface activating molecule/a ligand for an activating molecule on its surface, recruiting/instructing/interacting with at least one other cell (including other macrophages and/or T cells) to stimulate pro-inflammatory responses, presenting antigen in a pro-inflammatory context, migrating to the site allowing for pro-inflammatory response initiation or starting to express at least one gene that is expected to lead to pro-inflammatory functionality. In some embodiments, the term includes activating cytotoxic CD8+ T cells, mediating increased sensitivity of cancer cells to immunotherapy, such as immune checkpoint therapy, and/or mediating reversal of cancer cells to resistance. In certain embodiments, such modulation toward a pro-inflammatory state can be measured in a number of well-known manners, including, without limitation, one or more of a) increased cluster of differentiation 80 (CD80), CD86, MHCII, MHCI, interleukin 1-beta (IL-1β, IL-6, CCL3, CCL4, CXCL10, CXCL9, GM-CSF and/or tumor necrosis factor alpha (TNF-α); b) decreased expression of CD206, CD163, CD16, CD53, VSIG4, PSGL-1, TGFb and/or IL-10; c) increased secretion of at least one cytokine or chemokine selected from the group consisting of IL-1β, TNF-α, IL-12, IL-18, GM-CSF, CCL3, CCL4, and IL-23; d) increased ratio of expression of IL-1β, IL-6, and/or TNF-α to expression of IL-10; e) increased CD8+ cytotoxic T cell activation; f) increased recruitment of CD8+ cytotoxic T cell activation; g) increased CD4+ helper T cell activity; h) increased recruitment of CD4+ helper T cell activity; i) increased NK cell activity; j) increased recruitment of NK cell; k) increased neutrophil activity; 1) increased macrophage activity; and/or m) increased spindle-shaped morphology, flatness of appearance, and/or number of dendrites, as assessed by microscopy.

In cells that are already pro-inflammatory, an increased inflammatory phenotype refers to an even more pro-inflammatory state.

By contrast, the term “M2 macrophages” refers to macrophages having an anti-inflammatory phenotype. Th2- and tumor-derived cytokines, such as IL-4, IL-10, IL-13, transforming growth factor beta (TGF-β), or prostaglandin E2 (PGE2) can promulgate M2 polarization. The metabolic profile of M2 macrophages is defined by OXPHOS, FAO, a decreased glycolysis, and PPP. The discovery that the mannose receptor was selectively enhanced by the Th2 IL-4 and IL-13 in murine macrophages, and induced high endocytic clearance of mannosylated ligands, increased major histocompatibility complex (MHC) class II antigen expression, and reduced pro-inflammatory cytokine secretion, led Stein, Doyle, and colleagues to propose that IL-4 and IL-13 induced an alternative activation phenotype, a state altogether different from IFN-γ activation but far from deactivation (Martinez and Gordon (2014) F1000 Prime Reports 6:13). In vitro and in vivo definition/assays can measure different endpoints: general in vitro endpoints include anti-inflammatory cell stimulation measured by proliferation, migration, anti-inflammatory Th2 cytokine/chemokine secretion and/or migration, while general in vivo M2 endpoints further include analyzing pathogen fighting, tissue injury delayed/pro-fibrotic response, other cell Th2 polarization, migration inducers, etc. For both in vitro and in vivo, pro-tolerogenic antigen presentation can be assessed.

A “Type 2” or “M2-like” monocyte and/or macrophage is a monocyte and/or macrophage capable of contributing to an anti-inflammatory response that is characterized by at least one of the following: producing anti-inflammatory stimuli by secreting at least one anti-inflammatory cytokine, expressing at least one cell surface inhibiting molecule/ligand for an inhibitory molecule on its surface, recruiting/instructing/interacting at least one other cell to stimulate anti-inflammatory responses, presenting antigen in a pro-tolerogenic context, migrating to the site allowing for pro-tolerogenic response initiation or starting to express at least one gene that is expected to lead to pro-tolerogenic/anti-inflammatory functionality. In certain embodiments, such modulation toward a pro-inflammatory state can be measured in a number of well-known manners, including, without limitation, the opposite of the Type 1 pro-inflammatory state measurements described above.

A cell that has an “increased inflammatory phenotype” is one that has a more pro-inflammatory response capacity related to a) an increase in one or more of the Type 1 listed-criteria and/or b) a decrease in one or more of the Type 2-listed criteria, after modulation of at least one biomarker (e.g., at least one target listed in Table 1 and/or Table 2) of the present invention, such as contact by an agent that modulates the at least one biomarker (e.g., at least one target listed in Table 1 and/or Table 2) of the present invention.

A cell that has a “decreased inflammatory phenotype” is one that has a more anti-inflammatory response capacity related to a) an decrease in one or more of the Type 1 listed-criteria and/or b) an increase of one or more of the Type 2-listed criteria, after modulation of at least one biomarker (e.g., at least one target listed in Table 1 and/or Table 2) of the present invention, such as contact by an agent that modulates the at least one biomarker (e.g., at least one target listed in Table 1 and/or Table 2) of the present invention.

Thus, macrophages can adopt a continuum of alternatively activated states with intermediate phenotypes between the Type 1 and Type 2 states (see, e.g., Biswas et al. (2010) Nat. Immunol. 11: 889-896; Mosser and Edwards (2008) Nat. Rev. Immunol. 8:958-969; Mantovani et al. (2009) Hum. Immunol. 70:325-330) and such increased or decreased inflammatory phenotypes can be determined as described above.

As used herein, the term “alternatively activated macrophages” or “alternatively activated states” refers to essentially all types of macrophage populations other than the classically activated M1 pro-inflammatory macrophages. Originally, the alternatively activated state was designated only to M2 type anti-inflammatory macrophages. The term has expanded to include all other alternative activation states of macrophages with dramatic difference in their biochemistry, physiology and functionality.

For example, one type of alternatively activated macrophages is those involved in wound healing. In response to innate and adaptive signals released during tissue injury (e.g., surgical wound), such as IL-4 produced by basophils and mast cells, tissue-resident macrophages can be activated to promote wound healing. The wound healing macrophages, instead of producing high levels of pro-inflammatory cytokines, secret large amounts of extracellular matrix components, e.g., chitinase and chitinase-like proteins YM1/CHI3L3, YM2, AMCase and stabilin, all of which exhibit carbohydrate and matrix-binding activities and involve in tissue repair.

Another example of alternatively activated macrophages involves regulatory macrophages that can be induced by innate and adaptive immune response. Regulatory macrophages can contribute to immuno-regulatory function. For example, macrophages can respond to hormones from the hypothalamic-pituitary-adrenal (HPA) axis (e.g., glucocorticoids) to adopt a state with inhibited host defense and inflammatory function such as inhibition of the transcriptions of pro-inflammatory cytokines. Regulatory macrophages can produce regulatory cytokine TGF-β to dampen immune responses in certain conditions, for instance, at late stage of adaptive immune response. Many regulatory macrophages can express high levels of co-stimulatory molecules (e.g., CD80 and CD86) and therefore enhance antigen presentation to T cells.

Many stimuli/cues can induce polarization of regulatory macrophages. The cues can include, but are not limited to, the combination of TLR agonist and immune complexes, apoptotic cells, IL-10, prostaglandins, GPcR ligands, adenosine, dopamine, histamine, sphingosine1-phosphate, melanocortin, vasoactive intestinal peptides and Siglec-9. Some pathogens, such as parasites, viruses, and bacteria, can specifically induce the differentiation of regulatory macrophages, resulting in defective pathogen killing and enhanced survival and spread of the infected microorganisms.

Regulatory macrophages share some common features. For example, regulatory macrophages need two stimuli to induce their anti-inflammatory activity. Differences among the regulatory macrophage subpopulations that are induced by different cues/stimuli are also observed, reflecting their heterogeneity.

Regulatory macrophages also are a heterogeneous population of macrophages, including a variety of subpopulations found in metabolism, during development, in the maintenance of homeostasis. In one example, a subpopulation of alternatively activated macrophages are immunoregulatory macrophages with unique immunoregulatory properties which can be induced in the presence of M-CSF/GM-CSF, a CD16 ligand (such as an immunoglobulin), and IFN-γ (PCT Publ. No. WO 2017/153607).

Macrophages in a tissue can change their activation states in vivo over time. This dynamic reflects constant influx of migrating macrophages to the tissue, dynamic changes of activated macrophages, and macrophages that switch back the rest state. In some conditions, different signals in an environment can induce macrophages to a mix of different activation states. For example, in a condition with chronic wound, macrophages over time, can include pro-inflammatory activation subpopulation, macrophages that are pro-wound healing, and macrophages that exhibit some pro-resolving activities. Under non-pathological conditions, a balanced population of immune-stimulatory and immune-regulatory macrophages exist in the immune system. In some disease conditions, the balance is interrupted and the imbalance causes many clinical conditions.

The apparent plasticity of macrophages also make them vulnerably responsive to environmental cues they receive in a disease condition. Macrophages can be repolarized in response to a variety of disease conditions, demonstrating distinct characteristics. One example is macrophages that are attracted and filtrate into tumor tissues from peripheral blood monocytes, which are often called “tumor associated macrophages” (“TAMs”) or “tumor infiltrating macrophages” (“TIMs”). Tumor-associated macrophages are amongst the most abundant inflammatory cells in tumors and a significant correlation was found between high TAM density and a worse prognosis for most cancers (Zhang et al. (2012) PloS One 7:e50946.10.1371/journal.pone.0050946).

TAMs are a mixed population of both M1-like pro-inflammatory and M2-like anti-inflammatory subpopulations. In the earliest stage of neoplasia, classically activated macrophages that have a pro-inflammatory phenotype are present in the normoxic tumor regions, are believed to contribute to early eradication of transformed tumor cells. However, as a tumor grows and progresses, the majority of TAMs in late stage tumors is M2-like regulatory macrophages that reside in the hypoxic regions of the tumor. This phenotypic change of macrophages is markedly influenced by the tumor microenvironmental stimuli, such as tumor extracellular matrix, anoxic environment and cytokines secreted by tumor cells. The M2-like TAMs demonstrate a hybrid activation state of wound healing macrophages and regulatory macrophages, demonstrating various unique characteristics, including the production of high levels of IL-10 but little or no IL-12, defective TNF production, suppression of antigen presenting cells, and contribution to tumor angiogenesis.

Generally, TAMs are characterized by a M2 phenotype and suppress M1 macrophage-mediated inflammation through IL-10 and IL-1β production. Thus, TAMs promote tumor growth and metastasis through activation of wound-healing (i.e., anti-inflammatory) pathways that provide nutrients and growth signals for proliferation and invasion and promote the creation of new blood vessels (i.e., angiogenesis). In addition, TAMs contribute to the immune-suppressive tumor microenvironment by secreting anti-inflammatory signals that prevent other components of the immune system from recognizing and attacking the tumor. It has been reported that TAMs are key players in promoting cancer growth, proliferation, and metastasis in many types of cancers (e.g., breast cancer, astrocytoma, head and neck squamous cell cancer, papillary renal cell carcinoma Type II, lung cancer, pancreatic cancer, gall bladder cancer, rectal cancer, glioma, classical Hodgkin's lymphoma, ovarian cancer, and colorectal cancer). In general, a cancer characterized by a large population of TAMs is associated with poor disease prognosis.

The diversified functions and activation states can have dangerous consequences if not appropriately regulated. For example, classically activated macrophages can cause damage to host tissue, predispose surrounding tissue and influence glucose metabolism if over activated.

In many disease conditions, the balanced dynamics of macrophage activation states is interrupted and the imbalance causes diseases. For example, tumors are abundantly populated with macrophages. Macrophages can be found in 75 percent of cancers. The aggressive types of cancer are often associated with higher infiltration of macrophages and other immune cells. In most malignant tumors, TAM exert several tumor-promoting functions, including promotion of cancer cell survival, proliferation, invasion, extravasation and metastasis, stimulation of angiogenesis, remodeling of the extracellular matrix, and suppression of antitumor immunity (Qian and Pollard, 2010, Cell, 141(1): 39-51). They also could produce growth-promoting molecules such as ornithine, VEGF, EGF and TGF-β.

TAMs stimulate tumor growth and survival in response to CSF1 and IL4/IL13 encountered in the tumor microenvironment. TAMs also can remodel the tumor microenvironment through the expression of proteases, such as MMPs, cathepsins and uPA and matrix remodeling enzymes (e.g., lysyl oxidase and SPARC).

TAMs play an important role in tumor angiogenesis regulating the dramatic increase of blood vessel in tumor tissues which is required for the transition of the malignant state of tumor. These angiogenic TAMs express angiopoietin receptor, TIE2 and secrete many angiogenic molecules including VEGF family members, TNFα, IL1β, IL8, PDGF and FGF.

A diversity of subpopulations of macrophages perform these individual pro-tumoral functions. These TAMs are different in the extent of macrophage infiltrate as well as phenotype in different tumor types. For example, detailed profiling in human hepatocellular carcinoma shows various macrophage sub-types defined in terms of their anatomic location, and pro-tumoral and anti-tumoral properties. It has been shown that M2-like macrophages are a major resource of pro-tumoral functions of TAMs. M2-like TAMs have been shown to affect the efficacy of anti-cancer treatments, contribute to therapy resistance, and mediate tumor relapse following conventional cancer therapy.

Dysregulated monocytes and/or macrophages have been found in a variety of disorders such as autoimmune diseases, chronic inflammation, multiple sclerosis, rheumatoid arthritis, atherosclerosis, Type I diabetes, Type II diabetes, obesity, allergy, asthma, hemophagocytic lymphohistiocytosis, sarcoidosis, periodontitis, pulmonary alveolar proteinosis, macrophage-related pulmonary disease, cardiovascular diseases, microbial infection, transplant-related complications, metabolic syndrome, hypertension, and inflammatory neurological diseases. Monocytes and macrophages are potential therapeutic targets for those macrophage mediated diseases.

### CCR2 and CSF1R as Myeloid-Derived Cell Targets

CCR2 (C—C chemokine receptor 2; also known as CCR2A, CCR2B, CD192, CMKBR2 and CKR2) is a G protein-coupled receptor expressed on cell surface that can be activated by multiple chemokines known as macrophage chemoattractant proteins including CCL2 (MCP-1), CCL8 (MCP-2), CCL7 (MCP-3), CCL13 (MCP-4) and CCL16 in human (Charo et al. (1994) Proc. Natl. Acad. Sci. U.S.A. 91:2752-2756). Activation of CCR2 results in directional migration of receptor-bearing cell types such as monocytes, dendritic cells and macrophages. CCR2 plays an important role in immune cell trafficking, especially for recruiting circulating bone marrow derived monocytes to inflammatory sites and subsequent transformation to macrophages or dendritic cells. For example, CCR2 activation is deeply involved in cancer metastatic process by increasing the migration and invasion of monocytes from the bone marrow to cancer tissues). It has been shown that tumor cells can express CCL2, which attracts CCR2-positive monocytes and macrophages to the tumor area. The infiltrated macrophages, under the influence of tumor microenvironments, are adapted to tumor-promoting functions. CCR2 signaling cascades are also involved in numerous inflammatory diseases and neurodegenerative disorders, as well as cardiovascular disorders such as atherosclerosis and myocardial infarction (see Franca et al. (2017) Clin. Sci. 131:1215-1224). CCR2 is also a co-receptor for HIV (Conner et al. (1997) J. Exp. Med. 185:621-628).

Due to the involvement of CCR2 and its ligand CCL2/MCP-1 in undesirable immune responses in various diseases, it has been recognized that CCR2 antagonists are promising therapeutic agents in preventing, treating, or ameliorating a macrophage-mediated inflammatory disease, such as cancer. For example, a CCR antagonist can suppress the proliferation, migration and invasion of human lung adenocarcinoma cells (An et al. (2017) Oncotarget 8:39230-39240). Blocking CCL2/CCR2 axis can suppress TAMs and activate anti-tumor immune response in cancers such as hepatocellular carcinoma (Li et al. (2017) Gut 66:157-167).

CSF1R (colony stimulating factor 1 receptor; also known as macrophage colony-stimulating factor receptor (M-CSFR), FMS, FIM2, C-FMS, and CD115 in the art) is a single-pass transmembrane receptor with an N-terminal extracellular domain (ECD) and a C-terminal intracellular domain with tyrosine kinase activity. Activation of CSF1R by the ligand CSF1 (also known as M-CSF) or IL-34 can stimulate the trafficking, survival, proliferation, and differentiation of monocytes and macrophages. Dysregulation of CSF1R activity can result in an imbalance in the levels and/or activities of macrophage cell populations, which can lead to several diseases. Expression and activity of CSF1R has been shown to be important for sustaining tumor infiltrating macrophages (TAMs) in a variety of solid tumor types and hematologic malignancies (e.g., chronic lymphocytic leukemia (CLL)).

Both CSF1R and its ligand CSF1 have been identified as potential therapeutic targets for many macrophage-mediated diseases, including cancer, autoimmune diseases, and inflammation. It has been reported that CSF1R inhibition can deplete the suppressive tumor micro-environmental signal from CD4⁺ monocytes in AML (Edwards et al. (2015) Blood 126:3824). Several studies also showed that CSF1 and/or CSF1R inhibitors, such as siRNAs, antagonist antibodies, and small molecule inhibitors (e.g., GW2580) can reverse immune-inhibitory TAMs in pancreatic cancer (Zhu et al. (2014) Cancer Res. 74:5057-5069), diffuse-type giant cell tumor (Dt-GCT) (Ries et al. (2014) Cancer Cell 25:846-859), and acute myeloid leukemia (AML) (Moughon et al. (2015) Cancer Res. 75:4742-4752). The blockage of TAMs by inhibiting CSF1R activation can effectively improve cancer treatment in tested tumor models. CSF1R and CSF1 antagonists such as antibodies directed against CSF1R and CSF1 interaction, RNAi mediated silencing of CSF1R or CSF1 expression (e.g., PCT Publ. No. WO 2007/081879), soluble forms of the CSF1R extracellular domain (ECD) (see e.g., WO 2007/081879), and small molecule inhibitors of CSF1R tyrosine kinase activity, and inhibitors of CSF1 have been investigated for treatment of macrophage mediated diseases (see, e.g., PCT Publ. No. WO 2007/081879; Irvine et al. (2006) FASEB J. 20:1315-1326; Ohno et al. (2008) Clin. Immunol. 38: 283-291).

Without being bound by theory, it is believed that the use of a combination of oligonucleotide compositions described herein and formulations comprising same is particularly effective to inhibit CCR2 and CSF1R activation in order to simultaneously inhibit the trafficking, polarization and activation of monocytes and macrophages in response to an environmental signal, such as a growth factor from tumor cells.

### III. Compositions of the Invention

Antagonists of CCR2 and CSF1R have been investigated for their effects on modulating macrophage content in a disease condition, for example the content of pro-tumorigenic macrophages (e.g., TAMs) and pro-inflammatory macrophages that inhibit tumorigenesis. The present invention provides compositions comprising particularly effective antagonists of CCR2 and/or CSF1R that block CCR2 and CSF1R signaling and that can functions synergistically to block CCR2 and CSF1R simultaneously.

In accordance with the present invention, antagonists can be small molecules, peptidomimetics, polypeptides, peptides, antibodies, nucleic acid molecules in either sense or anti-sense orientation, either single or double stranded nucleic acids specifically targeted to CCR2 and/or CSF1R. In some embodiments, a nucleic acid-based agent can be a single molecule that targets both CCR2 and CSF1R by comprising complementary sequences (e.g., anti-sense) against CCR2 and CSF1R, such as by separation using an oligonucleotide linker. In some embodiments, nucleic acid-based agents individually target either CCR2 or CSF1R. In some embodiments, the combined antagonists of CCR2 and CSF1R comprise double stranded siRNA molecule cocktail.

One aspect encompassed by the present invention involves the use of nucleic acid molecules. Nucleic acid molecules can be deoxyribonucleic acid (DNA) molecules (e.g., cDNA, genomic DNA, and the like), ribonucleic acid (RNA) molecules (e.g., mRNA, long non-coding RNA, small RNA species, and the like), DNA/RNA hybrids, and analogs of the DNA or RNA generated using nucleotide analogs. RNA agents can include RNAi (RNA interfering) agents (e.g., small interfering RNA (siRNA)), single-strand RNA (ssRNA) molecules (e.g., antisense oligonucleotides) or double-stranded RNA (dsRNA) molecules. A dsRNA molecule comprises a first strand and a second strand, wherein the second strand is substantially complementary to the first strand, and the first strand and the second strand form at least one double-stranded duplex region. The dsRNA molecule can be blunt-ended or have at least one terminal overhang. When used as agents that bind target nucleic acid sequences, nucleic acid agents encompassed by the present invention can n hybridize to any region of a target sequence, such as genomic sequence and/or mRNA sequence, including, but not limited to, the enhancer region, the promoter region, the transcriptional start and/or stop region, splice sites, the coding region, the 3′-untranslated region (3′-UTR), the 5′-untranslated region (5′-UTR), the 5′ cap, the 3′ poly adenylyl tail, or any combination thereof.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A nucleic acid molecule encompassed by the present invention can be isolated using standard molecular biology techniques and the sequence information in the database records described herein. Using all or a portion of such nucleic acid sequences, nucleic acid molecules encompassed by the present invention can be isolated using standard hybridization and cloning techniques (e.g., as described in Sambrook et al., ed., Molecular Cloning: A Laboratory Manual, 4th ed., Cold Spring Harbor Laboratory Press, Cold Spring Harbor, N.Y., 2012).

A nucleic acid molecule encompassed by the present invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, nucleic acid molecules corresponding to all or a portion of a nucleic acid molecule encompassed by the present invention can be prepared by standard synthetic techniques, e.g., using an automated nucleic acid synthesizer. Alternatively, the nucleic acid molecules can be produced biologically using an expression vector into which a nucleic acid has been sub-cloned. For example, antisense nucleic acid molecules can be cloned in an antisense orientation (i.e., RNA transcribed from the inserted nucleic acid will be of an antisense orientation to a target nucleic acid of interest as described further below).

Moreover, a nucleic acid molecule encompassed by the present invention can comprise only a portion of a nucleic acid sequence, wherein the full length nucleic acid sequence comprises a marker encompassed by the present invention or which encodes a polypeptide corresponding to a marker encompassed by the present invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a biomarker nucleic acid sequence. Probes based on the sequence of a biomarker nucleic acid molecule can be used to detect transcripts or genomic sequences corresponding to one or more markers encompassed by the present invention. The probe comprises a label group attached thereto, e.g., a radioisotope, a fluorescent compound, an enzyme, or an enzyme co-factor.

Biomarker nucleic acid molecules that differ, due to degeneracy of the genetic code, from the nucleotide sequence of nucleic acid molecules encoding a protein which corresponds to the biomarker, and thus encode the same protein, are also contemplated.

In addition, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that can affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. For example, biomarker alleles can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's can occur in protein-coding nucleic acid sequences, in which case they can give rise to a defective or otherwise variant protein, or genetic disease. Such a SNP can alter the coding sequence of the gene and therefore specify another amino acid (a “missense” SNP) or a SNP can introduce a stop codon (a “nonsense” SNP). When a SNP does not alter the amino acid sequence of a protein, the SNP is called “silent.” SNP's can also occur in noncoding regions of the nucleotide sequence. This can result in defective protein expression, e.g., as a result of alternative spicing, or it can have no effect on the function of the protein.

As used herein, the terms “gene” and “recombinant gene” refer to nucleic acid molecules comprising an open reading frame encoding a polypeptide corresponding to a marker encompassed by the present invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope encompassed by the present invention.

In another embodiment, a biomarker nucleic acid molecule can be at least 7, 15, 20, 25, 30, 40, 60, 80, 100, 150, 200, 250, 300, 350, 400, 450, 550, 650, 700, 800, 900, 1000, 1100, 1200, 1300, 1400, 1500, 1600, 1700, 1800, 1900, 2000, 2200, 2400, 2600, 2800, 3000, 3500, 4000, 4500, or more nucleotides in length and hybridizes under stringent conditions to a nucleic acid molecule corresponding to a marker encompassed by the present invention or to a nucleic acid molecule encoding a protein corresponding to a marker encompassed by the present invention. The term “hybridizes under stringent conditions” is intended to describe conditions for hybridization and washing under which nucleotide sequences at least 60% (65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

In addition to naturally-occurring allelic variants of a nucleic acid molecule encompassed by the present invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species can be non-essential for activity and thus would be likely targets for alteration. Alternatively, amino acid residues that are conserved among the homologs of various species (e.g., murine and human) can be essential for activity and thus would not be likely targets for alteration.

Accordingly, another aspect encompassed by the present invention encompasses nucleic acid molecules encoding a polypeptide encompassed by the present invention that contain changes in amino acid residues that are not essential for activity. Such polypeptides differ in amino acid sequence from the naturally-occurring proteins which correspond to the markers encompassed by the present invention, yet retain biological activity. In one embodiment, a biomarker protein has an amino acid sequence that is at least about 40% identical, 50%, 60%, 70%, 75%, 80%, 83%, 85%, 87.5%, 90%, 91%, 92%, 93%, 94%, 95%, 96%, 97%, 98%, 99% or identical to the amino acid sequence of a biomarker protein described herein.

An isolated nucleic acid molecule encoding a variant protein can be created by introducing one or more nucleotide substitutions, additions or deletions into the nucleotide sequence of nucleic acids encompassed by the present invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

As described further below, some forms of nucleic acids useful according to the present invention can act as inhibitors, which refers to an agent that inhibits the function of a biological target. In some embodiments, the inhibitor is a gene silencing agent that prevents the expression of a gene or gene product. “Gene silencing” is often referred to as “gene knockdown.” Gene silencing can occur on the transcriptional level, i.e., prevent the transcription of DNA to RNA, or on the translational level, i.e., post-transcriptional silencing i.e., prevent the translation of mRNA to protein. Types of transcriptional gene silencing include genomic imprinting, paramutation, transposon silencing, histone modification, transgene silencing, position effect, and RNA-directed DNA methylation, for example. Examples of post-transcriptional gene silencing include RNA interference (RNAi), RNA silencing, and nonsense mediated decay. A gene silencing agent can be designed to silence (e.g., inhibit the expression of) a specific gene or to silence multiple genes simultaneously. A gene silencing agent can reduce the expression of a gene and/or gene product by at least about 20%, at least about 30%, at least about 40%, at least about 50%, at least about 60%, at least about 70%, at least about 80%, at least about 90%, at least about 95%, at least about 98%, at least about 99%, or at least about 100%. In some embodiments, a gene silencing agent reduces expression of a gene and/or gene product by at least about 70%.

In some embodiments, nucleic acids in genomes are useful and can be used as targets and/or agents. For example, target DNA in the genome can be manipulated using well-known methods in the art. Target DNA in the genome can be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, can be altered by site-directed mutagenesis.

siRNA Molecules

In some embodiments, the antagonists of CCR2 and CSF1R are small interfering RNA (siRNA) molecules that hybridize to CCR2 or CSF1R. In other embodiments, the antagonists of CCR2 and CSF1R can be shRNA (short hairpin RNA) molecules in which the two strands of the siRNA molecule can be connected by a linker region (e.g., a nucleotide linker or a non-nucleotide linker).

The siRNA molecules specific to CCR2 can hybridize to human CCR2 mRNA, including the coding region, the untranslated regions and UTRs (Gene Bank Ref. Sequence NM_001123041.2; SEQ ID NO: 1). The siRNA molecules specific to CCR2 can target all protein coding transcripts of CCR2 (e.g., CCR2 isoforms CCR2A (NM_001123041.2; SEQ ID NO: 1) and CCR2B (NM_001123396.1; SEQ ID NO: 3)) and its orthologs, such as in cynomolgus and rhesus monkey.

The siRNA molecules specific to CSF1R can hybridize to human CSF1R mRNA including the coding region, the untranslated regions and UTRs (Gene Bank Ref. Sequence NM_005211.3; SEQ ID NO: 2). The siRNA molecules specific to CSF1R2 can target all protein coding transcripts of CSF1R2 (e.g., CSF1R isoform 1 (NM_005211.3; SEQ ID NO: 2) and CSF1R isoform 2 (NM_001288705.2; SEQ ID NO: 4) and CSF1R isoform 4 (NM_001349736.1; SEQ ID NO: 5) and its orthologs, such as in cynomolgus and rhesus monkey.

TABLE 1
Representative CCR2 and CSF1R cDNA sequences

| | | SEQ |
|---|---|---|
| | | ID |
| Description | Sequence (5′-3′) | NO: |
| Human | TTTATTCTCTGGAACATGAAACATTCTGTTGTGCTCATATCATGCAAATT | 1 |
| CCR2 | ATCACTAGTAGGAGAGCAGAGAGTGGAAATGTTCCAGGTATAAAGACCC | |
| mRNA | ACAAGATAAAGAAGCTCAGAGTCGTTAGAAACAGGAGCAGATGTACAG | |
| NM_001123041.2 | GGTTTGCCTGACTCACACTCAAGGTTGCATAAGCAAGATTTCAAAATTAA | |
| | TCCTATTCTGGAGACCTCAACCCAATGTACAATGTTCCTGACTGGAAAAG | |
| | AAGAACTATATTTTTCTGATTTTTTTTTTCAAATCTTTACCATTAGTTGCC | |
| | CTGTATCTCCGCCTTCACTTTCTGCAGGAAACTTTATTTCCTACTTCTGCA | |
| | TGCCAAGTTTCTACCTCTAGATCTGTTTGGTTCAGTTGCTGAGAAGCCTG | |
| | ACATACCAGGACTGCCTGAGACAAGCCACAAGCTGAACAGAGAAAGTG | |
| | GATTGAACAAGGACGCATTTCCCCAGTACATCCACAACATGCTGTCCAC | |
| | ATCTCGTTCTCGGTTTATCAGAAATACCAACGAGAGCGGTGAAGAAGTC | |
| | ACCACCTTTTTTGATTATGATTACGGTGCTCCCTGTCATAAATTTGACGTG | |
| | AAGCAAATTGGGGCCCAACTCCTGCCTCCGCTCTACTCGCTGGTGTTCAT | |
| | CTTTGGTTTTGTGGGCAACATGCTGGTCGTCCTCATCTTAATAAACTGCA | |
| | AAAAGCTGAAGTGCTTGACTGACATTTACCTGCTCAACCTGGCCATCTCT | |
| | GATCTGCTTTTTCTTATTACTCTCCCATTGTGGGCTCACTCTGCTGCAAAT | |
| | GAGTGGGTCTTTGGGAATGCAATGTGCAAATTATTCACAGGGCTGTATC | |
| | ACATCGGTTATTTTGGCGGAATCTTCTTCATCATCCTCCTGACAATCGAT | |
| | AGATACCTGGCTATTGTCCATGCTGTGTTTGCTTTAAAAGCCAGGACGGT | |
| | CACCTTTGGGGTGGTGACAAGTGTGATCACCTGGTTGGTGGCTGTGTTTG | |
| | CTTCTGTCCCAGGAATCATCTTTACTAAATGCCAGAAAGAAGATTCTGTT | |
| | TATGTCTGTGGCCCTTATTTTCCACGAGGATGGAATAATTTCCACACAAT | |
| | AATGAGGAACATTTTGGGGCTGGTCCTGCCGCTGCTCATCATGGTCATCT | |
| | GCTACTCGGGAATCCTGAAAACCCTGCTTCGGTGTCGAAACGAGAAGAA | |
| | GAGGCATAGGGCAGTGAGAGTCATCTTCACCATCATGATTGTTTACTTTC | |
| | TCTTCTGGACTCCCTATAATATTGTCATTCTCCTGAACACCTTCCAGGAAT | |
| | TCTTCGGCCTGAGTAACTGTGAAAGCACCAGTCAACTGGACCAAGCCAC | |
| | GCAGGTGACAGAGACTCTTGGGATGACTCACTGCTGCATCAATCCCATC | |
| | ATCTATGCCTTCGTTGGGGAGAAGTTCAGAAGCCTTTTTCACATAGCTCT | |
| | TGGCTGTAGGATTGCCCCACTCCAAAAACCAGTGTGTGGAGGTCCAGGA | |
| | GTGAGACCAGGAAAGAATGTGAAAGTGACTACACAAGGACTCCTCGATG | |
| | GTCGTGGAAAAGGAAAGTCAATTGGCAGAGCCCCTGAAGCCAGTCTTCA | |
| | GGACAAAGAAGGAGCCTAGAGACAGAAATGACAGATCTCTGCTTTGGA | |
| | AATCACACGTCTGGCTTCACAGATGTGTGATTCACAGTGTGAATCTTGGT | |
| | GTCTACGTTACCAGGCAGGAAGGCTGAGAGGAGAGAGACTCCAGCTGG | |
| | GTTGGAAAACAGTATTTTCCAAACTACCTTCCAGTTCCTCATTTTTGAAT | |
| | ACAGGCATAGAGTTCAGACTTTTTTTAAATAGT | |
| | AAAAATAAAATTAAAGCTGAAAACTGCAACTTGTAAATGTGGTAAAGAG | |
| | TTAGTTTGAGTTACTATCATGTCAAACGTGAAAATGCTGTATTAGTCACA | |
| | GAGATAATTCTAGCTTTGAGCTTAAGAATTTTGAGCAGGTGGTATGTTTG | |
| | GGAGACTGCTGAGTCAACCCAATAGTTGTTGATTGGCAGGAGTTGGAAG | |
| | TGTGTGATCTGTGGGCACATTAGCCTATGTGCATGCAGCATCTAAGTAAT | |
| | GATGTCGTTTGAATCACAGTATACGCTCCATCGCTGTCATCTCAGCTGGA | |
| | TCTCCATTCTCTCAGGCTTGCTGCCAAAAGCCTTTTGTGTTTTGTTTTGTA | |
| | TCATTATGAAGTCATGCGTTTAATCACATTCGAGTGTTTCAGTGCTTCGC | |
| | AGATGTCCTTGATGCTCATATTGTTCCCTATTTTGCCAGTGGGAACTCCT | |
| | AAATCAAGTTGGCTTCTAATCAAAGCTTTTAAACCCTATTGGTAAAGAAT | |
| | GGAAGGTGGAGAAGCTCCCTGAAGTAAGCAAAGACTTTCCTCTTAGTCG | |
| | AGCCAAGTTAAGAATGTTCTTATGTTGCCCAGTGTGTTTCTGATCTGATG | |
| | CAAGCAAGAAACACTGGGCTTCTAGAACCAGGCAACTTGGGAACTAGAC | |
| | TCCCAAGCTGGACTATGGCTCTACTTTCAGGCCACATGGCTAAAGAAGG | |
| | TTTCAGAAAGAAGTGGGGACAGAGCAGAACTTTCACCTTCATATATTTGT | |
| | ATGATCCTAATGAATGCATAAAATGTTAAGTTGATGGTGATGAAATGTA | |
| | AATACTGTTTTTAACAACTATGATTTGGAAAATAAATCAATGCTATAACT | |
| | ATGTTGAAAAAAAAAAAAAAAAAA | |
| Human | GAAGGGCAGACAGAGTGTCCAAAAGCGTGAGAGCACGAAGTGAGGAGA | 2 |
| CSF1R | AGGTGGAGAAGAGAGAAGAGGAAGAGGAAGAGGAAGAGAGGAAGCGG | |
| (NM_005211.3) | AGGGAACTGCGGCCAGGCTAAAAGGGGAAGAAGAGGATCAGCCCAAGG | |
| | AGGAGGAAGAGGAAAACAAGACAAACAGCCAGTGCAGAGGAGAGGAA | |
| | CGTGTGTCCAGTGTCCCGATCCCTGCGGAGCTAGTAGCTGAGAGCTCTGT | |
| | GCCCTGGGCACCTTGCAGCCCTGCACCTGCCTGCCACTTCCCCACCGAGG | |
| | CCATGGGCCCAGGAGTTCTGCTGCTCCTGCTGGTGGCCACAGCTTGGCAT | |
| | GGTCAGGGAATCCCAGTGATAGAGCCCAGTGTCCCTGAGCTGGTCGTGA | |
| | AGCCAGGAGCAACGGTGACCTTGCGATGTGTGGGCAATGGCAGCGTGGA | |
| | ATGGGATGGCCCCCCATCACCTCACTGGACCCTGTACTCTGATGGCTCCA | |
| | GCAGCATCCTCAGCACCAACAACGCTACCTTCCAAAACACGGGGACCTA | |
| | TCGCTGCACTGAGCCTGGAGACCCCCTGGGAGGCAGCGCCGCCATCCAC | |
| | CTCTATGTCAAAGACCCTGCCCGGCCCTGGAACGTGCTAGCACAGGAGG | |
| | TGGTCGTGTTCGAGGACCAGGACGCACTACTGCCCTGTCTGCTCACAGAC | |
| | CCGGTGCTGGAAGCAGGCGTCTCGCTGGTGCGTGTGCGTGGCCGGCCCC | |
| | TCATGCGCCACACCAACTACTCCTTCTCGCCCTGGCATGGCTTCACCATC | |
| | CACAGGGCCAAGTTCATTCAGAGCCAGGACTATCAATGCAGTGCCCTGA | |
| | TGGGTGGCAGGAAGGTGATGTCCATCAGCATCCGGCTGAAAGTGCAGAA | |
| | AGTCATCCCAGGGCCCCCAGCCTTGACACTGGTGCCTGCAGAGCTGGTG | |
| | CGGATTCGAGGGGAGGCTGCCCAGATCGTGTGCTCAGCCAGCAGCGTTG | |
| | ATGTTAACTTTGATGTCTTCCTCCAACACAACAACACCAAGCTCGCAATC | |
| | CCTCAACAATCTGACTTTCATAATAACCGTTACCAAAAAGTCCTGACCCT | |
| | CAACCTCGATCAAGTAGATTTCCAACATGCCGGCAACTACTCCTGCGTGG | |
| | CCAGCAACGTGCAGGGCAAGCACTCCACCTCCATGTTCTTCCGGGTGGT | |
| | AGAGAGTGCCTACTTGAACTTGAGCTCTGAGCAGAACCTCATCCAGGAG | |
| | GTGACCGTGGGGGAGGGGCTCAACCTCAAAGTCATGGTGGAGGCCTACC | |
| | CAGGCCTGCAAGGTTTTAACTGGACCTACCTGGGACCCTTTTCTGACCAC | |
| | CAGCCTGAGCCCAAGCTTGCTAATGCTACCACCAAGGACACATACAGGC | |
| | ACACCTTCACCCTCTCTCTGCCCCGCCTGAAGCCCTCTGAGGCTGGCCGC | |
| | TACTCCTTCCTGGCCAGAAACCCAGGAGGCTGGAGAGCTCTGACGTTTG | |
| | AGCTCACCCTTCGATACCCCCCAGAGGTAAGCGTCATATGGACATTCATC | |
| | AACGGCTCTGGCACCCTTTTGTGTGCTGCCTCTGGGTACCCCCAGCCCAA | |
| | CGTGACATGGCTGCAGTGCAGTGGCCACACTGATAGGTGTGATGAGGCC | |
| | CAAGTGCTGCAGGTCTGGGATGACCCATACCCTGAGGTCCTGAGCCAGG | |
| | AGCCCTTCCACAAGGTGACGGTGCAGAGCCTGCTGACTGTTGAGACCTT | |
| | AGAGCACAACCAAACCTACGAGTGCAGGGCCCACAACAGCGTGGGGAG | |
| | TGGCTCCTGGGCCTTCATACCCATCTCTGCAGGAGCCCACACGCATCCCC | |
| | CGGATGAGTTCCTCTTCACACCAGTGGTGGTCGCCTGCATGTCCATCATG | |
| | GCCTTGCTGCTGCTGCTGCTCCTGCTGCTATTGTACAAGTATAAGCAGAA | |
| | GCCCAAGTACCAGGTCCGCTGGAAGATCATCGAGAGCTATGAGGGCAAC | |
| | AGTTATACTTTCATCGACCCCACGCAGCTGCCTTACAACGAGAAGTGGG | |
| | AGTTCCCCCGGAACAACCTGCAGTTTGGTAAGACCCTCGGAGCTGGAGC | |
| | CTTTGGGAAGGTGGTGGAGGCCACGGCCTTTGGTCTGGGCAAGGAGGAT | |
| | GCTGTCCTGAAGGTGGCTGTGAAGATGCTGAAGTCCACGGCCCATGCTG | |
| | ATGAGAAGGAGGCCCTCATGTCCGAGCTGAAGATCATGAGCCACCTGGG | |
| | CCAGCACGAGAACATCGTCAACCTTCTGGGAGCCTGTACCCATGGAGGC | |
| | CCTGTACTGGTCATCACGGAGTACTGTTGCTATGGCGACCTGCTCAACTT | |
| | TCTGCGAAGGAAGGCTGAGGCCATGCTGGGACCCAGCCTGAGCCCCGGC | |
| | CAGGACCCCGAGGGAGGCGTCGACTATAAGAACATCCACCTCGAGAAG | |
| | AAATATGTCCGCAGGGACAGTGGCTTCTCCAGCCAGGGTGTGGACACCT | |
| | ATGTGGAGATGAGGCCTGTCTCCACTTCTTCAAATGACTCCTTCTCTGAG | |
| | CAAGACCTGGACAAGGAGGATGGACGGCCCCTGGAGCTCCGGGACCTGC | |
| | TTCACTTCTCCAGCCAAGTAGCCCAGGGCATGGCCTTCCTCGCTTCCAAG | |
| | AATTGCATCCACCGGGACGTGGCAGCGCGTAACGTGCTGTTGACCAATG | |
| | GTCATGTGGCCAAGATTGGGGACTTCGGGCTGGCTAGGGACATCATGAA | |
| | TGACTCCAACTACATTGTCAAGGGCAATGCCCGCCTGCCTGTGAAGTGG | |
| | ATGGCCCCAGAGAGCATCTTTGACTGTGTCTACACGGTTCAGAGCGACG | |
| | TCTGGTCCTATGGCATCCTCCTCTGGGAGATCTTCTCACTTGGGCTGAAT | |
| | CCCTACCCTGGCATCCTGGTGAACAGCAAGTTCTATAAACTGGTGAAGG | |
| | ATGGATACCAAATGGCCCAGCCTGCATTTGCCCCAAAGAATATATACAG | |
| | CATCATGCAGGCCTGCTGGGCCTTGGAGCCCACCCACAGACCCACCTTCC | |
| | AGCAGATCTGCTCCTTCCTTCAGGAGCAGGCCCAAGAGGACAGGAGAGA | |
| | GCGGGACTATACCAATCTGCCGAGCAGCAGCAGAAGCGGTGGCAGCGG | |
| | CAGCAGCAGCAGTGAGCTGGAGGAGGAGAGCTCTAGTGAGCACCTGAC | |
| | CTGCTGCGAGCAAGGGGATATCGCCCAGCCCTTGCTGCAGCCCAACAAC | |
| | TATCAGTTCTGCTGAGGAGTTGACGACAGGGAGTACCACTCTCCCCTCCC | |
| | ACAAACTTCAACTCCTCCATGGATGGGGCGACACGGGGAGAACATACAA | |
| | ACTCTGCCTTCGGTCATTTCACTCAACAGCTCGGCCCAGCTCTGAAACTT | |
| | GGGAAGGTGAGGGATTCAGGGGAGGTCAGAGGATCCCACTTCCTGAGCA | |
| | TGGGCCATCACTGCCAGTCAGGGGCTGGGGGCTGAGCCCTCACCCCCCC | |
| | CTCCCCTACTGTTCTCATGGTGTTGGCCTCGTGTTTGCTATGCCAACTAGT | |
| | AGAACCTTCTTTCCTAATCCCCTTATCTTCATGGAAATGGACTGACTTTAT | |
| | GCCTATGAAGTCCCCAGGAGCTACACTGATACTGAGAAAACCAGGCTCT | |
| | TTGGGGCTAGACAGACTGGCAGAGAGTGAGATCTCCCTCTCTGAGAGGA | |
| | GCAGCAGATGCTCACAGACCACACTCAGCTCAGGCCCCTTGGAGCAGGA | |
| | TGGCTCCTCTAAGAATCTCACAGGACCTCTTAGTCTCTGCCCTATACGCC | |
| | GCCTTCACTCCACAGCCTCACCCCTCCCACCCCCATACTGGTACTGCTGT | |
| | AATGAGCCAAGTGGCAGCTAAAAGTTGGGGGTGTTCTGCCCAGTCCCGT | |
| | CATTCTGGGCTAGAAGGCAGGGGACCTTGGCATGTGGCTGGCCACACCA | |
| | AGCAGGAAGCACAAACTCCCCCAAGCTGACTCATCCTAACTAACAGTCA | |
| | CGCCGTGGGATGTCTCTGTCCACATTAAACTAACAGCATTAATGCAGTCA | |
| | AAAAAAAAAAAAAAAAA | |

* The nucleic acid sequences encompassed by the present invention listed in Table 1 have been submitted at GenBank under the unique identifier provided herein and each such uniquely identified sequence submitted at GenBank is hereby incorporated in its entirety by reference.
* Included in Table 1 are RNA nucleic acid molecules (e.g., thymidines replaced with uridines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any publicly available sequence listed in Table 1, or a portion thereof. Such nucleic acid molecules can have a function of the full-length nucleic acid as described further herein.
* Included in Table 1 are orthologs of the proteins encoded by the nucleic acid sequences, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any publicly available sequence listed in Table 2, or a portion thereof. Such polypeptides can have a function of the full-length polypeptide as described further herein.
* Included in Table 1 are additional known nucleic acid and amino acid sequences for the listed CCR2 and CSF1R biomarkers.

In some embodiments, the siRNA molecules encompassed by the present invention can comprise about 10 to 50 nucleotides or nucleotide analogs. The siRNA molecules encompassed by the present invention include a duplex region wherein the duplex region comprises (or consists of) a sense region and an antisense region that together form the duplex region. The antisense strand having sufficient complementarity to a target mRNA (e.g., CCR2 mRNA or CSF1R mRNA) to mediate RNAi. The siRNA molecule encompassed by the present invention can have a length from about 10-50 or more nucleotides, i.e., each strand comprises 10-50 nucleotides. Preferably, the sense and antisense strand of the siRNA molecule each has a length from about 15-45 nucleotides. Further preferably, the antisense and the sense strand of the siRNA molecule each has a length from 18 to 30 nucleotides, for example, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, or about 30 nucleotides, and the antisense region comprises (or alternatively, consists essentially of, or consists of) a nucleotide sequence that is substantially complementary to the target mRNA. As used herein, the term “substantially complementary” refers to complementarity in a based-paired and double stranded region of the siRNA molecule. The complementarity does not need to be perfect; there can be any number of base pair mismatches that do not impact hybridization under even the least stringent hybridization conditions. For example, the antisense region of the siRNA molecule encompassed by the present invention can comprise at least about 80% or greater complementary, or at least about 85% or greater complementary, or at least about 90% or greater complementary, or at least about 91% or greater complementary, or at least about 92% or greater complementary, or at least about 93% or greater complementary, or at least about 94% or greater complementary, or at least about 95% or greater complementary, or at least about 96% or greater complementary, or at least about 97% or greater complementary, or at least about 98% or greater complementary, or at least about 99% or greater complementary, to the nucleic acid sequence of the target mRNA molecule, for example the nucleic acid sequence of CCR2 mRNA (SEQ ID NO:1), or the nucleic acid sequence of CSF1R mRNA (SEQ ID NO: 2), to direct target specific RNA interference (RNAi).

In some embodiments, the siRNA molecules encompassed by the present invention can further include at least one overhang region, wherein each overhang region has six or fewer nucleotides. That is to say, when the antisense and sense strands of a siRNA molecule are aligned, there are at least one, two, three, four, five or six nucleotides at the end of the strands which do not align (i.e., no complementary bases in the opposing strand). In some examples, an overhang can occur at one or both ends of the duplex when the sense and antisense strands are annealed.

In some examples, the antisense region and the sense region of the siRNA molecule encompassed by the present invention can vary in lengths, sequences and the nature of chemical modifications thereto.

The siRNA molecule that hybridizes to CCR2 mRNA (SEQ ID NO: 1) can comprise a sense strand nucleic acid sequence selected from the group consisting of nucleic acid sequences of SEQ ID NOs: 6 to 67; and an antisense strand nucleic acid sequence selected from the group consisting of nucleic acid sequences of SEQ ID NOs: 68 to 129 (Table 2).

In some embodiments, the siRNA molecule that hybridizes to CCR2 mRNA (SEQ ID NO: 1) can comprise a sense strand nucleic acid sequence of SEQ ID NO: 6 and an antisense strand nucleic acid sequence of SEQ ID NO: 68; or a sense strand nucleic acid sequence of SEQ ID NO: 7 and an antisense strand nucleic acid sequence of SEQ ID NO: 69; or a sense strand nucleic acid sequence of SEQ ID NO: 8 and an antisense strand nucleic acid sequence of SEQ ID NO: 70; or a sense strand nucleic acid sequence of SEQ ID NO: 9 and an antisense strand nucleic acid sequence of SEQ ID NO: 71; or a sense strand nucleic acid sequence of SEQ ID NO: 10 and an antisense strand nucleic acid sequence of SEQ ID NO: 72; or a sense strand nucleic acid sequence of SEQ ID NO: 11 and an antisense strand nucleic acid sequence of SEQ ID NO: 73; or a sense strand nucleic acid sequence of SEQ ID NO: 12 and an antisense strand nucleic acid sequence of SEQ ID NO: 74; or a sense strand nucleic acid sequence of SEQ ID NO: 13 and an antisense strand nucleic acid sequence of SEQ ID NO: 75; or a sense strand nucleic acid sequence of SEQ ID NO: 14 and an antisense strand nucleic acid sequence of SEQ ID NO: 76; or a sense strand nucleic acid sequence of SEQ ID NO: 15 and an antisense strand nucleic acid sequence of SEQ ID NO: 77; or a sense strand nucleic acid sequence of SEQ ID NO: 16 and an antisense strand nucleic acid sequence of SEQ ID NO: 78; or a sense strand nucleic acid sequence of SEQ ID NO: 17 and an antisense strand nucleic acid sequence of SEQ ID NO: 79; or a sense strand nucleic acid sequence of SEQ ID NO: 18 and an antisense strand nucleic acid sequence of SEQ ID NO: 80; or a sense strand nucleic acid sequence of SEQ ID NO: 19 and an antisense strand nucleic acid sequence of SEQ ID NO: 81; or a sense strand nucleic acid sequence of SEQ ID NO: 20 and an antisense strand nucleic acid sequence of SEQ ID NO: 82; or a sense strand nucleic acid sequence of SEQ ID NO: 21 and an antisense strand nucleic acid sequence of SEQ ID NO: 83; or a sense strand nucleic acid sequence of SEQ ID NO: 22 and an antisense strand nucleic acid sequence of SEQ ID NO: 84; or a sense strand nucleic acid sequence of SEQ ID NO: 23 and an antisense strand nucleic acid sequence of SEQ ID NO: 85; or a sense strand nucleic acid sequence of SEQ ID NO: 24 and an antisense strand nucleic acid sequence of SEQ ID NO: 86; or a sense strand nucleic acid sequence of SEQ ID NO: 25 and an antisense strand nucleic acid sequence of SEQ ID NO: 87; or a sense strand nucleic acid sequence of SEQ ID NO: 26 and an antisense strand nucleic acid sequence of SEQ ID NO: 88; or a sense strand nucleic acid sequence of SEQ ID NO: 27 and an antisense strand nucleic acid sequence of SEQ ID NO: 89; or a sense strand nucleic acid sequence of SEQ ID NO: 28 and an antisense strand nucleic acid sequence of SEQ ID NO: 90; or a sense strand nucleic acid sequence of SEQ ID NO: 29 and an antisense strand nucleic acid sequence of SEQ ID NO: 91; or a sense strand nucleic acid sequence of SEQ ID NO: 30 and an antisense strand nucleic acid sequence of SEQ ID NO: 92; or a sense strand nucleic acid sequence of SEQ ID NO: 31 and an antisense strand nucleic acid sequence of SEQ ID NO: 93; or a sense strand nucleic acid sequence of SEQ ID NO: 32 and an antisense strand nucleic acid sequence of SEQ ID NO: 94; or a sense strand nucleic acid sequence of SEQ ID NO: 33 and an antisense strand nucleic acid sequence of SEQ ID NO: 95; or a sense strand nucleic acid sequence of SEQ ID NO: 34 and an antisense strand nucleic acid sequence of SEQ ID NO: 96; or a sense strand nucleic acid sequence of SEQ ID NO: 35 and an antisense strand nucleic acid sequence of SEQ ID NO: 97; or a sense strand nucleic acid sequence of SEQ ID NO: 36 and an antisense strand nucleic acid sequence of SEQ ID NO: 98; or a sense strand nucleic acid sequence of SEQ ID NO: 37 and an antisense strand nucleic acid sequence of SEQ ID NO: 99; or a sense strand nucleic acid sequence of SEQ ID NO: 38 and an antisense strand nucleic acid sequence of SEQ ID NO: 100; or a sense strand nucleic acid sequence of SEQ ID NO: 39 and an antisense strand nucleic acid sequence of SEQ ID NO: 101; or a sense strand nucleic acid sequence of SEQ ID NO: 40 and an antisense strand nucleic acid sequence of SEQ ID NO: 102; or a sense strand nucleic acid sequence of SEQ ID NO: 41 and an antisense strand nucleic acid sequence of SEQ ID NO: 103; or a sense strand nucleic acid sequence of SEQ ID NO: 42 and an antisense strand nucleic acid sequence of SEQ ID NO: 104; or a sense strand nucleic acid sequence of SEQ ID NO: 43 and an antisense strand nucleic acid sequence of SEQ ID NO: 105; or a sense strand nucleic acid sequence of SEQ ID NO: 44 and an antisense strand nucleic acid sequence of SEQ ID NO: 106; or a sense strand nucleic acid sequence of SEQ ID NO: 45 and an antisense strand nucleic acid sequence of SEQ ID NO: 107; or a sense strand nucleic acid sequence of SEQ ID NO: 46 and an antisense strand nucleic acid sequence of SEQ ID NO: 108; or a sense strand nucleic acid sequence of SEQ ID NO: 47 and an antisense strand nucleic acid sequence of SEQ ID NO: 109; or a sense strand nucleic acid sequence of SEQ ID NO: 48 and an antisense strand nucleic acid sequence of SEQ ID NO: 110; or a sense strand nucleic acid sequence of SEQ ID NO: 49 and an antisense strand nucleic acid sequence of SEQ ID NO: 111; or a sense strand nucleic acid sequence of SEQ ID NO: 50 and an antisense strand nucleic acid sequence of SEQ ID NO: 112; or a sense strand nucleic acid sequence of SEQ ID NO: 51 and an antisense strand nucleic acid sequence of SEQ ID NO: 113; or a sense strand nucleic acid sequence of SEQ ID NO: 52 and an antisense strand nucleic acid sequence of SEQ ID NO: 114; or a sense strand nucleic acid sequence of SEQ ID NO: 53 and an antisense strand nucleic acid sequence of SEQ ID NO: 115; or a sense strand nucleic acid sequence of SEQ ID NO: 54 and an antisense strand nucleic acid sequence of SEQ ID NO: 116; or a sense strand nucleic acid sequence of SEQ ID NO: 55 and an antisense strand nucleic acid sequence of SEQ ID NO: 117; or a sense strand nucleic acid sequence of SEQ ID NO: 56 and an antisense strand nucleic acid sequence of SEQ ID NO: 118; or a sense strand nucleic acid sequence of SEQ ID NO: 57 and an antisense strand nucleic acid sequence of SEQ ID NO: 119; or a sense strand nucleic acid sequence of SEQ ID NO: 58 and an antisense strand nucleic acid sequence of SEQ ID NO: 120; or a sense strand nucleic acid sequence of SEQ ID NO: 59 and an antisense strand nucleic acid sequence of SEQ ID NO: 121; or a sense strand nucleic acid sequence of SEQ ID NO: 60 and an antisense strand nucleic acid sequence of SEQ ID NO: 122; or a sense strand nucleic acid sequence of SEQ ID NO: 61 and an antisense strand nucleic acid sequence of SEQ ID NO: 123; or a sense strand nucleic acid sequence of SEQ ID NO: 62 and an antisense strand nucleic acid sequence of SEQ ID NO: 124; or a sense strand nucleic acid sequence of SEQ ID NO: 63 and an antisense strand nucleic acid sequence of SEQ ID NO: 125; or a sense strand nucleic acid sequence of SEQ ID NO: 64 and an antisense strand nucleic acid sequence of SEQ ID NO: 126; or a sense strand nucleic acid sequence of SEQ ID NO: 65 and an antisense strand nucleic acid sequence of SEQ ID NO: 127; or a sense strand nucleic acid sequence of SEQ ID NO: 66 and an antisense strand nucleic acid sequence of SEQ ID NO: 128; or a sense strand nucleic acid sequence of SEQ ID NO: 67 and an antisense strand nucleic acid sequence of SEQ ID NO: 129.

TABLE 2
Sense and anti-sense sequences of siRNA molecules specific to CCR2

| ID (position | | SEQ | | SEQ |
|---|---|---|---|---|
| in CCR2 | | ID | | ID |
| NM_001123041.2 | sense strand sequence (5′-3′) | NO: | antisense strand sequence (5′-3′) | NO: |
| 463 | CAUUUCCCCAGUACAUCCA | 6 | UGGAUGUACUGGGGAAAUG | 68 |
| 464 | AUUUCCCCAGUACAUCCAC | 7 | GUGGAUGUACUGGGGAAAU | 69 |
| 486 | AUGCUGUCCACAUCUCGUU | 8 | AACGAGAUGUGGACAGCAU | 70 |
| 487 | UGCUGUCCACAUCUCGUUC | 9 | GAACGAGAUGUGGACAGCA | 71 |
| 488 | GCUGUCCACAUCUCGUUCU | 10 | AGAACGAGAUGUGGACAGC | 72 |
| 489 | CUGUCCACAUCUCGUUCUC | 11 | GAGAACGAGAUGUGGACAG | 73 |
| 491 | GUCCACAUCUCGUUCUCGG | 12 | CCGAGAACGAGAUGUGGAC | 74 |
| 529 | AGAGCGGUGAAGAAGUCAC | 13 | GUGACUUCUUCACCGCUCU | 75 |
| 533 | CGGUGAAGAAGUCACCACC | 14 | GGUGGUGACUUCUUCACCG | 76 |
| 541 | AAGUCACCACCUUUUUUGA | 15 | UCAAAAAAGGUGGUGACUU | 77 |
| 544 | UCACCACCUUUUUUGAUUA | 16 | UAAUCAAAAAAGGUGGUGA | 78 |
| 545 | CACCACCUUUUUUGAUUAU | 17 | AUAAUCAAAAAAGGUGGUG | 79 |
| 546 | ACCACCUUUUUUGAUUAUG | 18 | CAUAAUCAAAAAAGGUGGU | 80 |
| 571 | GUGCUCCCUGUCAUAAAUU | 19 | AAUUUAUGACAGGGAGCAC | 81 |
| 572 | UGCUCCCUGUCAUAAAUUU | 20 | AAAUUUAUGACAGGGAGCA | 82 |
| 574 | CUCCCUGUCAUAAAUUUGA | 21 | UCAAAUUUAUGACAGGGAG | 83 |
| 577 | CCUGUCAUAAAUUUGACGU | 22 | ACGUCAAAUUUAUGACAGG | 84 |
| 579 | UGUCAUAAAUUUGACGUGA | 23 | UCACGUCAAAUUUAUGACA | 85 |
| 580 | GUCAUAAAUUUGACGUGAA | 24 | UUCACGUCAAAUUUAUGAC | 86 |
| 581 | UCAUAAAUUUGACGUGAAG | 25 | CUUCACGUCAAAUUUAUGA | 87 |
| 583 | AUAAAUUUGACGUGAAGCA | 26 | UGCUUCACGUCAAAUUUAU | 88 |
| 586 | AAUUUGACGUGAAGCAAAU | 27 | AUUUGCUUCACGUCAAAUU | 89 |
| 587 | AUUUGACGUGAAGCAAAUU | 28 | AAUUUGCUUCACGUCAAAU | 90 |
| 783 | CACUCUGCUGCAAAUGAGU | 29 | ACUCAUUUGCAGCAGAGUG | 91 |
| 799 | AGUGGGUCUUUGGGAAUGC | 30 | GCAUUCCCAAAGACCCACU | 92 |
| 800 | GUGGGUCUUUGGGAAUGCA | 31 | UGCAUUCCCAAAGACCCAC | 93 |
| 802 | GGGUCUUUGGGAAUGCAAU | 32 | AUUGCAUUCCCAAAGACCC | 94 |
| 808 | UUGGGAAUGCAAUGUGCAA | 33 | UUGCACAUUGCAUUCCCAA | 95 |
| 821 | GUGCAAAUUAUUCACAGGG | 34 | CCCUGUGAAUAAUUUGCAC | 96 |
| 833 | CACAGGGCUGUAUCACAUC | 35 | GAUGUGAUACAGCCCUGUG | 97 |
| 851 | CGGUUAUUUUGGCGGAAUC | 36 | GAUUCCGCCAAAAUAACCG | 98 |
| 853 | GUUAUUUUGGCGGAAUCUU | 37 | AAGAUUCCGCCAAAAUAAC | 99 |
| 854 | UUAUUUUGGCGGAAUCUUC | 38 | GAAGAUUCCGCCAAAAUAA | 100 |
| 855 | UAUUUUGGCGGAAUCUUCU | 39 | AGAAGAUUCCGCCAAAAUA | 101 |
| 856 | AUUUUGGCGGAAUCUUCUU | 40 | AAGAAGAUUCCGCCAAAAU | 102 |
| 857 | UUUUGGCGGAAUCUUCUUC | 41 | GAAGAAGAUUCCGCCAAAA | 103 |
| 858 | UUUGGCGGAAUCUUCUUCA | 42 | UGAAGAAGAUUCCGCCAAA | 104 |
| 965 | AAGUGUGAUCACCUGGUUG | 43 | CAACCAGGUGAUCACACUU | 105 |
| 968 | UGUGAUCACCUGGUUGGUG | 44 | CACCAACCAGGUGAUCACA | 106 |
| 969 | GUGAUCACCUGGUUGGUGG | 45 | CCACCAACCAGGUGAUCAC | 107 |
| 970 | UGAUCACCUGGUUGGUGGC | 46 | GCCACCAACCAGGUGAUCA | 108 |
| 974 | CACCUGGUUGGUGGCUGUG | 47 | CACAGCCACCAACCAGGUG | 109 |
| 1007 | AGGAAUCAUCUUUACUAAA | 48 | UUUAGUAAAGAUGAUUCCU | 110 |
| 1055 | UGGCCCUUAUUUUCCACGA | 49 | UCGUGGAAAAUAAGGGCCA | 111 |
| 1056 | GGCCCUUAUUUUCCACGAG | 50 | CUCGUGGAAAAUAAGGGCC | 112 |
| 1057 | GCCCUUAUUUUCCACGAGG | 51 | CCUCGUGGAAAAUAAGGGC | 113 |
| 1060 | CUUAUUUUCCACGAGGAUG | 52 | CAUCCUCGUGGAAAAUAAG | 114 |
| 1061 | UUAUUUUCCACGAGGAUGG | 53 | CCAUCCUCGUGGAAAAUAA | 115 |
| 1062 | UAUUUUCCACGAGGAUGGA | 54 | UCCAUCCUCGUGGAAAAUA | 116 |
| 1065 | UUUCCACGAGGAUGGAAUA | 55 | UAUUCCAUCCUCGUGGAAA | 117 |
| 1066 | UUCCACGAGGAUGGAAUAA | 56 | UUAUUCCAUCCUCGUGGAA | 118 |
| 1067 | UCCACGAGGAUGGAAUAAU | 57 | AUUAUUCCAUCCUCGUGGA | 119 |
| 1069 | CACGAGGAUGGAAUAAUUU | 58 | AAAUUAUUCCAUCCUCGUG | 120 |
| 1083 | AAUUUCCACACAAUAAUGA | 59 | UCAUUAUUGUGUGGAAAUU | 121 |
| 1085 | UUUCCACACAAUAAUGAGG | 60 | CCUCAUUAUUGUGUGGAAA | 122 |
| 1089 | CACACAAUAAUGAGGAACA | 61 | UGUUCCUCAUUAUUGUGUG | 123 |
| 1104 | AACAUUUUGGGGCUGGUCC | 62 | GGACCAGCCCCAAAAUGUU | 124 |
| 1294 | AAUUCUUCGGCCUGAGUAA | 63 | UUACUCAGGCCGAAGAAUU | 125 |
| 1295 | AUUCUUCGGCCUGAGUAAC | 64 | GUUACUCAGGCCGAAGAAU | 126 |
| 1296 | UUCUUCGGCCUGAGUAACU | 65 | AGUUACUCAGGCCGAAGAA | 127 |
| 1299 | UUCGGCCUGAGUAACUGUG | 66 | CACAGUUACUCAGGCCGAA | 128 |
| 1306 | UGAGUAACUGUGAAAGCAC | 67 | GUGCUUUCACAGUUACUCA | 129 |

The siRNA molecule that hybridizes to CSF1R mRNA (SEQ ID NO: 2) can comprise a sense strand nucleic acid sequence selected from the group consisting of nucleic acid sequences of SEQ ID NOs: 130 to 248; and an antisense strand nucleic acid sequence selected from the group consisting of nucleic acid sequences of SEQ ID NOs: 249 to 367 (Table 3).

In some embodiments, the siRNA molecule that hybridizes to CSF1R mRNA (SEQ ID NO: 2) can comprise a sense strand nucleic acid sequence of SEQ ID NO: 130 and an antisense strand nucleic acid sequence of SEQ ID NO: 249; or a sense strand nucleic acid sequence of SEQ ID NO: 131 and an antisense strand nucleic acid sequence of SEQ ID NO: 250; or a sense strand nucleic acid sequence of SEQ ID NO: 132 and an antisense strand nucleic acid sequence of SEQ ID NO: 251; or a sense strand nucleic acid sequence of SEQ ID NO: 133 and an antisense strand nucleic acid sequence of SEQ ID NO: 252; or a sense strand nucleic acid sequence of SEQ ID NO: 134 and an antisense strand nucleic acid sequence of SEQ ID NO: 253; or a sense strand nucleic acid sequence of SEQ ID NO: 135 and an antisense strand nucleic acid sequence of SEQ ID NO: 254; or a sense strand nucleic acid sequence of SEQ ID NO: 136 and an antisense strand nucleic acid sequence of SEQ ID NO: 255; or a sense strand nucleic acid sequence of SEQ ID NO: 137 and an antisense strand nucleic acid sequence of SEQ ID NO: 256; or a sense strand nucleic acid sequence of SEQ ID NO: 138 and an antisense strand nucleic acid sequence of SEQ ID NO: 257; or a sense strand nucleic acid sequence of SEQ ID NO: 139 and an antisense strand nucleic acid sequence of SEQ ID NO: 258; or a sense strand nucleic acid sequence of SEQ ID NO: 140 and an antisense strand nucleic acid sequence of SEQ ID NO: 259; or a sense strand nucleic acid sequence of SEQ ID NO: 141 and an antisense strand nucleic acid sequence of SEQ ID NO: 260; or a sense strand nucleic acid sequence of SEQ ID NO: 142 and an antisense strand nucleic acid sequence of SEQ ID NO: 261; a sense strand nucleic acid sequence of SEQ ID NO: 143 and an antisense strand nucleic acid sequence of SEQ ID NO: 262; or a sense strand nucleic acid sequence of SEQ ID NO: 144 and an antisense strand nucleic acid sequence of SEQ ID NO: 263; or a sense strand nucleic acid sequence of SEQ ID NO: 145 and an antisense strand nucleic acid sequence of SEQ ID NO: 264; or a sense strand nucleic acid sequence of SEQ ID NO: 146 and an antisense strand nucleic acid sequence of SEQ ID NO: 265; or a sense strand nucleic acid sequence of SEQ ID NO: 147 and an antisense strand nucleic acid sequence of SEQ ID NO: 266; or a sense strand nucleic acid sequence of SEQ ID NO: 148 and an antisense strand nucleic acid sequence of SEQ ID NO: 267; or a sense strand nucleic acid sequence of SEQ ID NO: 149 and an antisense strand nucleic acid sequence of SEQ ID NO: 268; or a sense strand nucleic acid sequence of SEQ ID NO: 150 and an antisense strand nucleic acid sequence of SEQ ID NO: 269; or a sense strand nucleic acid sequence of SEQ ID NO: 151 and an antisense strand nucleic acid sequence of SEQ ID NO: 270; or a sense strand nucleic acid sequence of SEQ ID NO: 152 and an antisense strand nucleic acid sequence of SEQ ID NO: 271; or a sense strand nucleic acid sequence of SEQ ID NO: 153 and an antisense strand nucleic acid sequence of SEQ ID NO: 272; or a sense strand nucleic acid sequence of SEQ ID NO: 154 and an antisense strand nucleic acid sequence of SEQ ID NO: 273; or a sense strand nucleic acid sequence of SEQ ID NO: 155 and an antisense strand nucleic acid sequence of SEQ ID NO: 274; or a sense strand nucleic acid sequence of SEQ ID NO: 156 and an antisense strand nucleic acid sequence of SEQ ID NO: 275; or a sense strand nucleic acid sequence of SEQ ID NO: 157 and an antisense strand nucleic acid sequence of SEQ ID NO: 276; or a sense strand nucleic acid sequence of SEQ ID NO: 158 and an antisense strand nucleic acid sequence of SEQ ID NO: 277; or a sense strand nucleic acid sequence of SEQ ID NO: 159 and an antisense strand nucleic acid sequence of SEQ ID NO: 278; or a sense strand nucleic acid sequence of SEQ ID NO: 160 and an antisense strand nucleic acid sequence of SEQ ID NO: 279; or a sense strand nucleic acid sequence of SEQ ID NO: 161 and an antisense strand nucleic acid sequence of SEQ ID NO: 280; or a sense strand nucleic acid sequence of SEQ ID NO: 162 and an antisense strand nucleic acid sequence of SEQ ID NO: 281; or a sense strand nucleic acid sequence of SEQ ID NO: 163 and an antisense strand nucleic acid sequence of SEQ ID NO: 282; or a sense strand nucleic acid sequence of SEQ ID NO: 164 and an antisense strand nucleic acid sequence of SEQ ID NO: 283; or a sense strand nucleic acid sequence of SEQ ID NO: 165 and an antisense strand nucleic acid sequence of SEQ ID NO: 284; or a sense strand nucleic acid sequence of SEQ ID NO: 166 and an antisense strand nucleic acid sequence of SEQ ID NO: 285; or a sense strand nucleic acid sequence of SEQ ID NO: 167 and an antisense strand nucleic acid sequence of SEQ ID NO: 286; or a sense strand nucleic acid sequence of SEQ ID NO: 168 and an antisense strand nucleic acid sequence of SEQ ID NO: 287; or a sense strand nucleic acid sequence of SEQ ID NO: 169 and an antisense strand nucleic acid sequence of SEQ ID NO: 288; or a sense strand nucleic acid sequence of SEQ ID NO: 170 and an antisense strand nucleic acid sequence of SEQ ID NO: 289; or a sense strand nucleic acid sequence of SEQ ID NO: 171 and an antisense strand nucleic acid sequence of SEQ ID NO: 290; or a sense strand nucleic acid sequence of SEQ ID NO: 172 and an antisense strand nucleic acid sequence of SEQ ID NO: 291; or a sense strand nucleic acid sequence of SEQ ID NO: 173 and an antisense strand nucleic acid sequence of SEQ ID NO: 292; or a sense strand nucleic acid sequence of SEQ ID NO: 174 and an antisense strand nucleic acid sequence of SEQ ID NO: 293; or a sense strand nucleic acid sequence of SEQ ID NO: 175 and an antisense strand nucleic acid sequence of SEQ ID NO: 294; or a sense strand nucleic acid sequence of SEQ ID NO: 176 and an antisense strand nucleic acid sequence of SEQ ID NO: 295; or a sense strand nucleic acid sequence of SEQ ID NO: 177 and an antisense strand nucleic acid sequence of SEQ ID NO: 296; or a sense strand nucleic acid sequence of SEQ ID NO: 178 and an antisense strand nucleic acid sequence of SEQ ID NO: 297; or a sense strand nucleic acid sequence of SEQ ID NO: 179 and an antisense strand nucleic acid sequence of SEQ ID NO: 298; or a sense strand nucleic acid sequence of SEQ ID NO: 180 and an antisense strand nucleic acid sequence of SEQ ID NO: 299; or a sense strand nucleic acid sequence of SEQ ID NO: 181 and an antisense strand nucleic acid sequence of SEQ ID NO: 300; or a sense strand nucleic acid sequence of SEQ ID NO: 182 and an antisense strand nucleic acid sequence of SEQ ID NO: 301; or a sense strand nucleic acid sequence of SEQ ID NO: 183 and an antisense strand nucleic acid sequence of SEQ ID NO: 302; or a sense strand nucleic acid sequence of SEQ ID NO: 184 and an antisense strand nucleic acid sequence of SEQ ID NO: 303; or a sense strand nucleic acid sequence of SEQ ID NO: 185 and an antisense strand nucleic acid sequence of SEQ ID NO: 304; or a sense strand nucleic acid sequence of SEQ ID NO: 186 and an antisense strand nucleic acid sequence of SEQ ID NO: 305; or a sense strand nucleic acid sequence of SEQ ID NO: 187 and an antisense strand nucleic acid sequence of SEQ ID NO: 306; or a sense strand nucleic acid sequence of SEQ ID NO: 188 and an antisense strand nucleic acid sequence of SEQ ID NO: 307; or a sense strand nucleic acid sequence of SEQ ID NO: 189 and an antisense strand nucleic acid sequence of SEQ ID NO: 308; or a sense strand nucleic acid sequence of SEQ ID NO: 190 and an antisense strand nucleic acid sequence of SEQ ID NO: 309; or a sense strand nucleic acid sequence of SEQ ID NO: 191 and an antisense strand nucleic acid sequence of SEQ ID NO: 310; or a sense strand nucleic acid sequence of SEQ ID NO: 192 and an antisense strand nucleic acid sequence of SEQ ID NO: 311; or a sense strand nucleic acid sequence of SEQ ID NO: 193 and an antisense strand nucleic acid sequence of SEQ ID NO: 312; or a sense strand nucleic acid sequence of SEQ ID NO: 194 and an antisense strand nucleic acid sequence of SEQ ID NO: 313; or a sense strand nucleic acid sequence of SEQ ID NO: 195 and an antisense strand nucleic acid sequence of SEQ ID NO: 314; or a sense strand nucleic acid sequence of SEQ ID NO: 196 and an antisense strand nucleic acid sequence of SEQ ID NO: 315; or a sense strand nucleic acid sequence of SEQ ID NO: 197 and an antisense strand nucleic acid sequence of SEQ ID NO: 316; or a sense strand nucleic acid sequence of SEQ ID NO: 198 and an antisense strand nucleic acid sequence of SEQ ID NO: 317; or a sense strand nucleic acid sequence of SEQ ID NO: 199 and an antisense strand nucleic acid sequence of SEQ ID NO: 318; or a sense strand nucleic acid sequence of SEQ ID NO: 200 and an antisense strand nucleic acid sequence of SEQ ID NO: 319; or a sense strand nucleic acid sequence of SEQ ID NO: 201 and an antisense strand nucleic acid sequence of SEQ ID NO: 320; or a sense strand nucleic acid sequence of SEQ ID NO: 202 and an antisense strand nucleic acid sequence of SEQ ID NO: 321; or a sense strand nucleic acid sequence of SEQ ID NO: 203 and an antisense strand nucleic acid sequence of SEQ ID NO: 322; or a sense strand nucleic acid sequence of SEQ ID NO: 204 and an antisense strand nucleic acid sequence of SEQ ID NO: 323; or a sense strand nucleic acid sequence of SEQ ID NO: 205 and an antisense strand nucleic acid sequence of SEQ ID NO: 324; or a sense strand nucleic acid sequence of SEQ ID NO: 206 and an antisense strand nucleic acid sequence of SEQ ID NO: 325; or a sense strand nucleic acid sequence of SEQ ID NO: 207 and an antisense strand nucleic acid sequence of SEQ ID NO: 326; or a sense strand nucleic acid sequence of SEQ ID NO: 208 and an antisense strand nucleic acid sequence of SEQ ID NO: 327; or a sense strand nucleic acid sequence of SEQ ID NO: 209 and an antisense strand nucleic acid sequence of SEQ ID NO: 328; or a sense strand nucleic acid sequence of SEQ ID NO: 210 and an antisense strand nucleic acid sequence of SEQ ID NO: 329; or a sense strand nucleic acid sequence of SEQ ID NO: 211 and an antisense strand nucleic acid sequence of SEQ ID NO: 330; or a sense strand nucleic acid sequence of SEQ ID NO: 212 and an antisense strand nucleic acid sequence of SEQ ID NO: 331; or a sense strand nucleic acid sequence of SEQ ID NO: 213 and an antisense strand nucleic acid sequence of SEQ ID NO: 332; or a sense strand nucleic acid sequence of SEQ ID NO: 214 and an antisense strand nucleic acid sequence of SEQ ID NO: 333; or a sense strand nucleic acid sequence of SEQ ID NO: 215 and an antisense strand nucleic acid sequence of SEQ ID NO: 334; or a sense strand nucleic acid sequence of SEQ ID NO: 216 and an antisense strand nucleic acid sequence of SEQ ID NO: 335; or a sense strand nucleic acid sequence of SEQ ID NO: 217 and an antisense strand nucleic acid sequence of SEQ ID NO: 336; or a sense strand nucleic acid sequence of SEQ ID NO: 218 and an antisense strand nucleic acid sequence of SEQ ID NO: 337; or a sense strand nucleic acid sequence of SEQ ID NO: 219 and an antisense strand nucleic acid sequence of SEQ ID NO: 338; or a sense strand nucleic acid sequence of SEQ ID NO: 220 and an antisense strand nucleic acid sequence of SEQ ID NO: 339; or a sense strand nucleic acid sequence of SEQ ID NO: 221 and an antisense strand nucleic acid sequence of SEQ ID NO: 340; or a sense strand nucleic acid sequence of SEQ ID NO: 222 and an antisense strand nucleic acid sequence of SEQ ID NO:341; or a sense strand nucleic acid sequence of SEQ ID NO: 223 and an antisense strand nucleic acid sequence of SEQ ID NO: 342; or a sense strand nucleic acid sequence of SEQ ID NO: 224 and an antisense strand nucleic acid sequence of SEQ ID NO: 343; or a sense strand nucleic acid sequence of SEQ ID NO: 225 and an antisense strand nucleic acid sequence of SEQ ID NO: 344; or a sense strand nucleic acid sequence of SEQ ID NO: 226 and an antisense strand nucleic acid sequence of SEQ ID NO: 345; or a sense strand nucleic acid sequence of SEQ ID NO: 227 and an antisense strand nucleic acid sequence of SEQ ID NO: 346; or a sense strand nucleic acid sequence of SEQ ID NO: 228 and an antisense strand nucleic acid sequence of SEQ ID NO: 347; or a sense strand nucleic acid sequence of SEQ ID NO: 229 and an antisense strand nucleic acid sequence of SEQ ID NO: 348; or a sense strand nucleic acid sequence of SEQ ID NO: 230 and an antisense strand nucleic acid sequence of SEQ ID NO: 349; or a sense strand nucleic acid sequence of SEQ ID NO: 231 and an antisense strand nucleic acid sequence of SEQ ID NO: 350; or a sense strand nucleic acid sequence of SEQ ID NO: 232 and an antisense strand nucleic acid sequence of SEQ ID NO: 351; or a sense strand nucleic acid sequence of SEQ ID NO: 233 and an antisense strand nucleic acid sequence of SEQ ID NO: 352; or a sense strand nucleic acid sequence of SEQ ID NO: 234 and an antisense strand nucleic acid sequence of SEQ ID NO: 353; or a sense strand nucleic acid sequence of SEQ ID NO: 235 and an antisense strand nucleic acid sequence of SEQ ID NO: 354; or a sense strand nucleic acid sequence of SEQ ID NO: 236 and an antisense strand nucleic acid sequence of SEQ ID NO: 355; or a sense strand nucleic acid sequence of SEQ ID NO: 237 and an antisense strand nucleic acid sequence of SEQ ID NO: 356; or a sense strand nucleic acid sequence of SEQ ID NO: 238 and an antisense strand nucleic acid sequence of SEQ ID NO: 357; or a sense strand nucleic acid sequence of SEQ ID NO: 239 and an antisense strand nucleic acid sequence of SEQ ID NO: 358; or a sense strand nucleic acid sequence of SEQ ID NO: 240 and an antisense strand nucleic acid sequence of SEQ ID NO: 359; or a sense strand nucleic acid sequence of SEQ ID NO: 241 and an antisense strand nucleic acid sequence of SEQ ID NO: 360; or a sense strand nucleic acid sequence of SEQ ID NO: 242 and an antisense strand nucleic acid sequence of SEQ ID NO: 361; or a sense strand nucleic acid sequence of SEQ ID NO: 243 and an antisense strand nucleic acid sequence of SEQ ID NO: 362; or a sense strand nucleic acid sequence of SEQ ID NO: 244 and an antisense strand nucleic acid sequence of SEQ ID NO: 363; or a sense strand nucleic acid sequence of SEQ ID NO: 245 and an antisense strand nucleic acid sequence of SEQ ID NO: 364; or a sense strand nucleic acid sequence of SEQ ID NO: 246 and an antisense strand nucleic acid sequence of SEQ ID NO: 365; or a sense strand nucleic acid sequence of SEQ ID NO: 247 and an antisense strand nucleic acid sequence of SEQ ID NO: 366; or a sense strand nucleic acid sequence of SEQ ID NO: 248 and an antisense strand nucleic acid sequence of SEQ ID NO: 367.

TABLE 3
Sense and anti-sense sequences of siRNA specific to CSF1R

| ID (Position | | SEQ | | SEQ |
|---|---|---|---|---|
| in CSF1R | core sense strand sequence | ID | core antisense strand sequence | ID |
| NM_005211.3) | (5′-3′) | NO: | (5′-3′) | NO: |
| 346 | GGGAAUCCCAGUGAUAGAG | 130 | CUCUAUCACUGGGAUUCCC | 249 |
| 410 | UUGCGAUGUGUGGGCAAUG | 131 | CAUUGCCCACACAUCGCAA | 250 |
| 412 | GCGAUGUGUGGGCAAUGGC | 132 | GCCAUUGCCCACACAUCGC | 251 |
| 508 | CAACGCUACCUUCCAAAAC | 133 | GUUUUGGAAGGUAGCGUUG | 252 |
| 510 | ACGCUACCUUCCAAAACAC | 134 | GUGUUUUGGAAGGUAGCGU | 253 |
| 511 | CGCUACCUUCCAAAACACG | 135 | CGUGUUUUGGAAGGUAGCG | 254 |
| 604 | UGCCCGGCCCUGGAACGUG | 136 | CACGUUCCAGGGCCGGGCA | 255 |
| 675 | UGCUCACAGACCCGGUGCU | 137 | AGCACCGGGUCUGUGAGCA | 256 |
| 700 | AGGCGUCUCGCUGGUGCGU | 138 | ACGCACCAGCGAGACGCCU | 257 |
| 817 | UCAAUGCAGUGCCCUGAUG | 139 | CAUCAGGGCACUGCAUUGA | 258 |
| 859 | CAGCAUCCGGCUGAAAGUG | 140 | CACUUUCAGCCGGAUGCUG | 259 |
| 921 | CAGAGCUGGUGCGGAUUCG | 141 | CGAAUCCGCACCAGCUCUG | 260 |
| 923 | GAGCUGGUGCGGAUUCGAG | 142 | CUCGAAUCCGCACCAGCUC | 261 |
| 930 | UGCGGAUUCGAGGGGAGGC | 143 | GCCUCCCCUCGAAUCCGCA | 262 |
| 948 | CUGCCCAGAUCGUGUGCUC | 144 | GAGCACACGAUCUGGGCAG | 263 |
| 950 | GCCCAGAUCGUGUGCUCAG | 145 | CUGAGCACACGAUCUGGGC | 264 |
| 953 | CAGAUCGUGUGCUCAGCCA | 146 | UGGCUGAGCACACGAUCUG | 265 |
| 1053 | AUAACCGUUACCAAAAAGU | 147 | ACUUUUUGGUAACGGUUAU | 266 |
| 1055 | AACCGUUACCAAAAAGUCC | 148 | GGACUUUUUGGUAACGGUU | 267 |
| 1056 | ACCGUUACCAAAAAGUCCU | 149 | AGGACUUUUUGGUAACGGU | 268 |
| 1060 | UUACCAAAAAGUCCUGACC | 150 | GGUCAGGACUUUUUGGUAA | 269 |
| 1288 | AGGUUUUAACUGGACCUAC | 151 | GUAGGUCCAGUUAAAACCU | 270 |
| 1289 | GGUUUUAACUGGACCUACC | 152 | GGUAGGUCCAGUUAAAACC | 271 |
| 1292 | UUUAACUGGACCUACCUGG | 153 | CCAGGUAGGUCCAGUUAAA | 272 |
| 1293 | UUAACUGGACCUACCUGGG | 154 | CCCAGGUAGGUCCAGUUAA | 273 |
| 1300 | GACCUACCUGGGACCCUUU | 155 | AAAGGGUCCCAGGUAGGUC | 274 |
| 1467 | UGACGUUUGAGCUCACCCU | 156 | AGGGUGAGCUCAAACGUCA | 275 |
| 1468 | GACGUUUGAGCUCACCCUU | 157 | AAGGGUGAGCUCAAACGUC | 276 |
| 1473 | UUGAGCUCACCCUUCGAUA | 158 | UAUCGAAGGGUGAGCUCAA | 277 |
| 1476 | AGCUCACCCUUCGAUACCC | 159 | GGGUAUCGAAGGGUGAGCU | 278 |
| 1485 | UUCGAUACCCCCCAGAGGU | 160 | ACCUCUGGGGGGUAUCGAA | 279 |
| 1488 | GAUACCCCCCAGAGGUAAG | 161 | CUUACCUCUGGGGGGUAUC | 280 |
| 1715 | GAGACCUUAGAGCACAACC | 162 | GGUUGUGCUCUAAGGUCUC | 281 |
| 1717 | GACCUUAGAGCACAACCAA | 163 | UUGGUUGUGCUCUAAGGUC | 282 |
| 1759 | CAGCGUGGGGAGUGGCUCC | 164 | GGAGCCACUCCCCACGCUG | 283 |
| 1815 | AUCCCCCGGAUGAGUUCCU | 165 | AGGAACUCAUCCGGGGGAU | 284 |
| 1819 | CCCGGAUGAGUUCCUCUUC | 166 | GAAGAGGAACUCAUCCGGG | 285 |
| 1847 | GUGGUCGCCUGCAUGUCCA | 167 | UGGACAUGCAGGCGACCAC | 286 |
| 1896 | UGCUAUUGUACAAGUAUAA | 168 | UUAUACUUGUACAAUAGCA | 287 |
| 1931 | CAGGUCCGCUGGAAGAUCA | 169 | UGAUCUUCCAGCGGACCUG | 288 |
| 1933 | GGUCCGCUGGAAGAUCAUC | 170 | GAUGAUCUUCCAGCGGACC | 289 |
| 1935 | UCCGCUGGAAGAUCAUCGA | 171 | UCGAUGAUCUUCCAGCGGA | 290 |
| 1936 | CCGCUGGAAGAUCAUCGAG | 172 | CUCGAUGAUCUUCCAGCGG | 291 |
| 1946 | AUCAUCGAGAGCUAUGAGG | 173 | CCUCAUAGCUCUCGAUGAU | 292 |
| 1948 | CAUCGAGAGCUAUGAGGGC | 174 | GCCCUCAUAGCUCUCGAUG | 293 |
| 1958 | UAUGAGGGCAACAGUUAUA | 175 | UAUAACUGUUGCCCUCAUA | 294 |
| 1962 | AGGGCAACAGUUAUACUUU | 176 | AAAGUAUAACUGUUGCCCU | 295 |
| 1964 | GGCAACAGUUAUACUUUCA | 177 | UGAAAGUAUAACUGUUGCC | 296 |
| 1990 | CACGCAGCUGCCUUACAAC | 178 | GUUGUAAGGCAGCUGCGUG | 297 |
| 2021 | UUCCCCCGGAACAACCUGC | 179 | GCAGGUUGUUCCGGGGGAA | 298 |
| 2026 | CCGGAACAACCUGCAGUUU | 180 | AAACUGCAGGUUGUUCCGG | 299 |
| 2042 | UUUGGUAAGACCCUCGGAG | 181 | CUCCGAGGGUCUUACCAAA | 300 |
| 2044 | UGGUAAGACCCUCGGAGCU | 182 | AGCUCCGAGGGUCUUACCA | 301 |
| 2145 | UGAAGUCCACGGCCCAUGC | 183 | GCAUGGGCCGUGGACUUCA | 302 |
| 2146 | GAAGUCCACGGCCCAUGCU | 184 | AGCAUGGGCCGUGGACUUC | 303 |
| 2264 | CCUGUACUGGUCAUCACGG | 185 | CCGUGAUGACCAGUACAGG | 304 |
| 2265 | CUGUACUGGUCAUCACGGA | 186 | UCCGUGAUGACCAGUACAG | 305 |
| 2266 | UGUACUGGUCAUCACGGAG | 187 | CUCCGUGAUGACCAGUACA | 306 |
| 2268 | UACUGGUCAUCACGGAGUA | 188 | UACUCCGUGAUGACCAGUA | 307 |
| 2272 | GGUCAUCACGGAGUACUGU | 189 | ACAGUACUCCGUGAUGACC | 308 |
| 2276 | AUCACGGAGUACUGUUGCU | 190 | AGCAACAGUACUCCGUGAU | 309 |
| 2277 | UCACGGAGUACUGUUGCUA | 191 | UAGCAACAGUACUCCGUGA | 310 |
| 2279 | ACGGAGUACUGUUGCUAUG | 192 | CAUAGCAACAGUACUCCGU | 311 |
| 2283 | AGUACUGUUGCUAUGGCGA | 193 | UCGCCAUAGCAACAGUACU | 312 |
| 2288 | UGUUGCUAUGGCGACCUGC | 194 | GCAGGUCGCCAUAGCAACA | 313 |
| 2292 | GCUAUGGCGACCUGCUCAA | 195 | UUGAGCAGGUCGCCAUAGC | 314 |
| 2295 | AUGGCGACCUGCUCAACUU | 196 | AAGUUGAGCAGGUCGCCAU | 315 |
| 2305 | GCUCAACUUUCUGCGAAGG | 197 | CCUUCGCAGAAAGUUGAGC | 316 |
| 2308 | CAACUUUCUGCGAAGGAAG | 198 | CUUCCUUCGCAGAAAGUUG | 317 |
| 2313 | UUCUGCGAAGGAAGGCUGA | 100 | UCAGCCUUCCUUCGCAGAA | 318 |
| 2444 | CAGGGUGUGGACACCUAUG | 200 | CAUAGGUGUCCACACCCUG | 319 |
| 2518 | GGACAAGGAGGAUGGACGG | 201 | CCGUCCAUCCUCCUUGUCC | 320 |
| 2600 | GCUUCCAAGAAUUGCAUCC | 202 | GGAUGCAAUUCUUGGAAGC | 321 |
| 2679 | ACUUCGGGCUGGCUAGGGA | 203 | UCCCUAGCCAGCCCGAAGU | 322 |
| 2687 | CUGGCUAGGGACAUCAUGA | 204 | UCAUGAUGUCCCUAGCCAG | 323 |
| 2689 | GGCUAGGGACAUCAUGAAU | 205 | AUUCAUGAUGUCCCUAGCC | 324 |
| 2774 | UUUGACUGUGUCUACACGG | 206 | CCGUGUAGACACAGUCAAA | 325 |
| 2776 | UGACUGUGUCUACACGGUU | 207 | AACCGUGUAGACACAGUCA | 326 |
| 2778 | ACUGUGUCUACACGGUUCA | 208 | UGAACCGUGUAGACACAGU | 327 |
| 2781 | GUGUCUACACGGUUCAGAG | 209 | CUCUGAACCGUGUAGACAC | 328 |
| 2785 | CUACACGGUUCAGAGCGAC | 210 | GUCGCUCUGAACCGUGUAG | 329 |
| 2788 | CACGGUUCAGAGCGACGUC | 211 | GACGUCGCUCUGAACCGUG | 330 |
| 2791 | GGUUCAGAGCGACGUCUGG | 212 | CCAGACGUCGCUCUGAACC | 331 |
| 2793 | UUCAGAGCGACGUCUGGUC | 213 | GACCAGACGUCGCUCUGAA | 332 |
| 2843 | CUUGGGCUGAAUCCCUACC | 214 | GGUAGGGAUUCAGCCCAAG | 333 |
| 2874 | UGAACAGCAAGUUCUAUAA | 215 | UUAUAGAACUUGCUGUUCA | 334 |
| 2880 | GCAAGUUCUAUAAACUGGU | 216 | ACCAGUUUAUAGAACUUGC | 335 |
| 2940 | AGAAUAUAUACAGCAUCAU | 217 | AUGAUGCUGUAUAUAUUCU | 336 |
| 3047 | AGAGAGCGGGACUAUACCA | 218 | UGGUAUAGUCCCGCUCUCU | 337 |
| 3048 | GAGAGCGGGACUAUACCAA | 219 | UUGGUAUAGUCCCGCUCUC | 338 |
| 3049 | AGAGCGGGACUAUACCAAU | 220 | AUUGGUAUAGUCCCGCUCU | 339 |
| 3052 | GCGGGACUAUACCAAUCUG | 221 | CAGAUUGGUAUAGUCCCGC | 340 |
| 3055 | GGACUAUACCAAUCUGCCG | 222 | CGGCAGAUUGGUAUAGUCC | 341 |
| 3149 | UGCUGCGAGCAAGGGGAUA | 223 | UAUCCCCUUGCUCGCAGCA | 342 |
| 3153 | GCGAGCAAGGGGAUAUCGC | 224 | GCGAUAUCCCCUUGCUCGC | 343 |
| 3154 | CGAGCAAGGGGAUAUCGCC | 225 | GGCGAUAUCCCCUUGCUCG | 344 |
| 3164 | GAUAUCGCCCAGCCCUUGC | 226 | GCAAGGGCUGGGCGAUAUC | 345 |
| 3186 | AGCCCAACAACUAUCAGUU | 227 | AACUGAUAGUUGUUGGGCU | 346 |
| 3187 | GCCCAACAACUAUCAGUUC | 228 | GAACUGAUAGUUGUUGGGC | 347 |
| 3188 | CCCAACAACUAUCAGUUCU | 229 | AGAACUGAUAGUUGUUGGG | 348 |
| 3292 | ACAAACUCUGCCUUCGGUC | 230 | GACCGAAGGCAGAGUUUGU | 349 |
| 3298 | UCUGCCUUCGGUCAUUUCA | 231 | UGAAAUGACCGAAGGCAGA | 350 |
| 3301 | GCCUUCGGUCAUUUCACUC | 232 | GAGUGAAAUGACCGAAGGC | 351 |
| 3305 | UCGGUCAUUUCACUCAACA | 233 | UGUUGAGUGAAAUGACCGA | 352 |
| 3457 | CUCAUGGUGUUGGCCUCGU | 234 | ACGAGGCCAACACCAUGAG | 353 |
| 3458 | UCAUGGUGUUGGCCUCGUG | 235 | CACGAGGCCAACACCAUGA | 354 |
| 3459 | CAUGGUGUUGGCCUCGUGU | 236 | ACACGAGGCCAACACCAUG | 355 |
| 3462 | GGUGUUGGCCUCGUGUUUG | 237 | CAAACACGAGGCCAACACC | 356 |
| 3473 | CGUGUUUGCUAUGCCAACU | 238 | AGUUGGCAUAGCAAACACG | 357 |
| 3808 | AGCUAAAAGUUGGGGGUGU | 239 | ACACCCCCAACUUUUAGCU | 358 |
| 3911 | CCAAGCUGACUCAUCCUAA | 240 | UUAGGAUGAGUCAGCUUGG | 359 |
| 3914 | AGCUGACUCAUCCUAACUA | 241 | UAGUUAGGAUGAGUCAGCU | 360 |
| 3916 | CUGACUCAUCCUAACUAAC | 242 | GUUAGUUAGGAUGAGUCAG | 361 |
| 3917 | UGACUCAUCCUAACUAACA | 243 | UGUUAGUUAGGAUGAGUCA | 362 |
| 3918 | GACUCAUCCUAACUAACAG | 244 | CUGUUAGUUAGGAUGAGUC | 363 |
| 3924 | UCCUAACUAACAGUCACGC | 245 | GCGUGACUGUUAGUUAGGA | 364 |
| 3925 | CCUAACUAACAGUCACGCC | 246 | GGCGUGACUGUUAGUUAGG | 365 |
| 3958 | UCCACAUUAAACUAACAGC | 247 | GCUGUUAGUUUAAUGUGGA | 366 |
| 3959 | CCACAUUAAACUAACAGCA | 248 | UGCUGUUAGUUUAAUGUGG | 367 |

Chemical Modifications of siRNA Molecules

In some embodiments, nucleic acid molecules encompassed by the present invention can contain one or more chemical modifications. The modifications will not compromise the activity of the nucleic acid molecules. Chemical modifications well-known in the art are capable of increasing stability, availability, and/or cell uptake of the nucleic acid molecules. In one embodiment, modifications can be used to provide improved resistance to degradation (by nucleases) or improved uptake of nucleic acid molecules by cells. In some embodiments, modified nucleic acid molecules encompassed by the present invention can have an enhanced target efficiency as compared to corresponding non-modified nucleic acid molecules.

In some embodiments, nucleic acid molecules encompassed by the present invention can be optimized, such as to increase expression, improve the effectiveness of gene silencing for use to silence a target gene, and the like. In another embodiment, modifications can be used to increase or decrease affinity for the complementary nucleotides in the target mRNA and/or in the complementary siRNA strand. In some embodiments, siRNAs encompassed by the present invention can be modified to increase the ability to avoid or modulate an immune response in a cell, tissue or organism.

In some embodiments, nucleic acid molecules encompassed by the present invention can be further modified to increase the membrane penetrance and/or delivery to a target organ, tissue and cell. In one example, the nucleic acid molecule can be modified to increase its delivery to myeloid cells, monocytes and macrophages. For example, nucleic acid molecules can be modified such that they specifically bind to receptors or antigens expressed on a selected cell surface, e.g., by linking the antisense nucleic acid molecules to peptides or antibodies which bind to cell surface receptors or antigens. The nucleic acid molecules can also be modified as part of vectors that target cells of interest and/or selectively express within cells of interest.

Duplex molecules encompassed by the present invention, such as siRNA molecules, can comprise a modified sense strand, a modified anti-sense strand, or modified sense and antisense strands.

In some embodiments, a nucleic acid molecule encompassed by the present invention can be an α-anomeric nucleic acid molecule. An α-anomeric nucleic acid molecule forms specific double-stranded hybrids with complementary RNA in which, contrary to the usual α-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

Nucleic acid molecules encompassed by the present invention can be modified at the 5′ end, 3′ end, 5′ and 3′ end, and/or at internal residues, or any combination thereof. As described herein, a naturally occurring nucleic acid with repeating nucleotide residues has a backbone consisting of sugars and phosphodiesters, and nitrogenous bases (often called nucleobases or simply bases). Accordingly chemically modified nucleotides can include modified nucleobases, modified sugars and/or non-phosphodiester linkages (i.e., backbone modifications). In some embodiments, the modification is a mixture of different kinds of modifications described herein, such as a combination of unlocked nucleomonomer agents (UNAs), modified cap structures, modified inter-nucleoside linkages and or nucleobase modifications.

### End/Cap Modifications

In some embodiments, nucleic acid molecules encompassed by the present invention can further comprise at least one terminal modification or “cap.”

For example, the cap can be a 5′ and/or a 3′-cap structure. The terms “cap” and “end-cap” include chemical modifications at either terminus of each strand of the nucleic acid molecule (with respect to terminal ribonucleotides), and/or modifications at the linkage between the last two nucleotides at the 5′ end and/or the last two nucleotides at the 3′ end. The cap structure can increase resistance of the nucleic acid molecule to exonucleases without compromising molecular interactions with target mRNAs or cellular machinery. Such modifications can be selected on the basis of their increased potency in vitro or in vivo.

The cap can be present at the 5′-terminus (5′-cap) or at the 3′-terminus (3′-cap) or can be present at both ends. In certain embodiments, the 5′- and/or 3′-cap is independently selected from phosphorothioate monophosphate, abasic residue (moiety), phosphorothioate linkage, 4′-thio nucleotide, carbocyclic nucleotide, phosphorodithioate linkage, inverted nucleotide or inverted abasic moiety (2′-3′ or 3′-3′) (e.g., Invabasic X, Abasic II, rSpacer/RNA abasic), and dSpacer), phosphorodithioate monophosphate, and methylphosphonate moiety. The phosphorothioate or phosphorodithioate linkage(s), when part of a cap structure, are generally positioned between the two terminal nucleotides at the 5′ end and the two terminal nucleotides at the 3′ end.

In some embodiments, nucleic acid molecules encompassed by the present invention have at least one terminal phosphorothioate monophosphate. The phosphorothioate monophosphate can be at the 5′ and/or 3′ end of each strand of the nucleic acid molecule. In other embodiments, the nucleic acid molecule has terminal phosphorothioate monophosphate at both 5′ and 3′ terminus of the sense and/or antisense strand. The phosphorothioate monophosphate can support a higher potency by inhibiting the action of exonucleases.

In some embodiments, modifications at the 5′ end is preferred in the sense strand, and comprises, for example, a 5′-propylamine group. Modifications to the 3′ OH terminus are in the sense strand, antisense strand, or in the sense and antisense strands. A 3′ end modification comprises, for example, 3′-puromycin, 3′-biotin and the like.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof. Exemplary 5′-modifications include, but are not limited to, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc.), 5′-alkyletherphosphonates (R(OH)(O)P—O-5′, R=alkylether, e.g., methoxymethyl (CH₂OMe), ethoxymethyl, etc.).

In some embodiments, the cap at the terminus of the nucleic acid molecule can be a conjugate, for example, a 5′ conjugate. The 5′ end conjugates can inhibit 5′ to 3′ exonucleolytic cleavage (e.g., naproxen; ibuprofen; small alkyl chains; aryl groups; heterocyclic conjugates; modified sugars (D-ribose, deoxyribose, glucose etc.)).

### Modified Nucleobases (Bases)

In some embodiments, nucleic acid molecules encompassed by the present invention can include base modifications and/or substitutions of natural nucleobases.

The term “unmodified” or “natural” nucleobases include the purine bases adenine (A) and guanine (G), and the pyrimidine bases thymine (T), cytosine (C) and uracil (U). In some embodiments, nucleic acid molecules can comprise one or more nucleobase-modified nucleotides. It can comprise about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, abut 27, about 28, about 29, or more nucleobase-modified nucleotides. In some examples, nucleic acid molecules can comprise about 1% to 10% modified nucleotides, or about 10% to 50% modified nucleotides. Modified bases refer to nucleotide bases such as, for example, adenine (A), guanine (G), cytosine (C), thymine (T), uracil (U), xanthine, inosine, and queuosine that have been modified by the replacement or addition of one or more atoms or groups. Some examples of types of modifications that can comprise nucleotides that are modified with respect to the base moieties include but are not limited to, alkylated, halogenated, thiolated, aminated, amidated, or acetylated bases, individually or in combination. More specific examples include, for example, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine, 5-propynyluridine, 5-propynylcytidine, 6-methyladenine, 6-methylguanine, N,N,-dimethyladenine, 2-propyladenine, 2-propylguanine, 2-aminoadenine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any 0- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties can be, or be based on, mannoses, arabinoses, glucopyranoses, galactopyranoses, 4′-thioribose, and other sugars, heterocycles, or carbocycles.

Exemplary modified nucleobases include, but are not limited to, other synthetic and naturally modified nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. In some particular embodiments, nucleobase-modified nucleotides useful in the invention include, but are not limited to: 5-bromo-uridine, 5-iodo-uridine, 5-methyl-cytidine, ribo-thymidine, 2-aminopurine, 5-fluoro-cytidine, and 5-fluoro-uridine, 2,6-diaminopurine, 4-thio-uridine; and 5-amino-allyl-uridine and the like.

In some embodiments, nucleic acid molecules encompassed by the present invention can also contain nucleotides with base analogues.

The nucleobase can be naturally occurring non canon bases such as CpG islands, inosine which can base pair with C, U or A, thiouridine, dihydrouridine, queuosine, xanthine, hypoxanthine, nubularine, isoguanisine, tubercidine and wyosine. Other analogues can include fluorophores (e.g., rhodamine, fluorescein) and other fluorescent base analogues such as 2-AP (2-aminopurine), 3-MI, 6-MI, 6-MAP, pyrrolo-dC, modified and improved derivatives of pyrrolo-dC, furan-modified bases, and tricyclic cytosine family (e.g., 1,3-Diaza-2-oxophenothiazine, tC; oxo-homologue of tC, tC^{O}; 1,3-diaza-2-oxophenoxazine). Nucleobase modified nucleotides can also include universal bases. By way of example, universal bases include but are not limited to 3-nitropyrrole, 5-nitroindole, or nebularine. The term “nucleotide” is also meant to include the N3′ to P5′ phosphoramidate, resulting from the substitution of a ribosyl 3′ oxygen with an amine group. As used herein, a universal nucleobase is any modified nucleobase that can base pair with all of the four naturally occurring nucleobases without substantially affecting the melting behavior, recognition by intracellular enzymes or activity of the oligonucleotide duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenzyl, tetracenyl, pentacenyl, and structural derivatives thereof. In some embodiments, the nucleotides of the nucleic acid molecules can incorporate base analogues and modified bases that are described in U.S. Pat. Nos. 6,008,334; 6,107,039; 6,664,058; 7,678,894; 7,786,292; and 7,956,171; U.S. Pat. Publ. Nos. 2013/122,506 and 2013/0296402; carboxamido-modified bases as described in PCT Pat. Publ. No. WO 2012/061810).

### Artificial Nucleic Acid Analogues (or Nucleotide Analogues)

In some embodiments, modified nucleic acid molecules encompassed by the present invention can comprise artificial nucleic acid analogues.

The term “nucleoside” refers to a molecule having a purine or pyrimidine base covalently linked to a ribose or deoxyribose sugar. Exemplary nucleosides include adenosine, guanosine, cytidine, uridine and thymidine.

The term “nucleotide” refers to a nucleoside having one or more phosphate groups joined in ester linkages to the sugar moiety. A nucleotide can be a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs.

The term “nucleotide analog”, also referred to herein as an “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function. Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

An analog can have any of the phosphate backbone, sugar, or the nucleobase (i.e., G, C, T, U, and A) altered. In some embodiments, the modified nucleotide can be an unlocked nucleomonomer agent (UNA). UNAs include any monomer unit suitable for inclusion in an oligomeric or polymeric composition such as an oligonucleotide or polynucleotide and which have, in reference to nucleosides or nucleotides, an unlocked or acyclic sugar moiety. Where such UNAs are included in a larger oligomer or polymer, such larger oligomer or polymer, e.g., oligonucleotide, can also be referred to as a UNA oligomer or UNA polymer, or UNA oligonucleotide. Where a UNA is included in a standard nucleotide, such variant nucleotide is referred to as a UNA nucleotide. Where a UNA is included in a standard nucleoside, such variant nucleoside is referred to as a UNA nucleoside. UNAs can be used as substitutes for nucleosides or nucleotides in oligonucleotides. In this case, UNAs, whether the monomer or oligomer containing the monomer, have often been referred to as “unlocked nucleic acids” in the art. When referred to as an unlocked nucleic acid herein, one of skill will understand that the inventors are referring to UNAs. According to the present invention, UNAs are not naturally occurring nucleomonomer agents. In one embodiment, one or more nucleotides in the nucleic acid molecule can be replaced with one or more unlocked nucleic acid/nuclomonomer agent (UNA) moieties, including those described in, e.g., PCT Publ. WO 2015/148580. A UNA oligomer can be a chain composed of UNA monomers, as well as various nucleotides that can be based on naturally-occurring nucleosides or modified nucleotides. UNA oligomers have been reported to have reduced off-target effects as compared to counterpart oligonucleotides lacking the modifications. Other UNA modifications and uses which can be utilized in accordance with the present invention include any of those disclosed in U.S. Pat. Publ. 2015/0232851, 2015/0232849, 2015/0239926, 2015/0239834, and 2015/0141678; U.S. Pat. No. 9,051,570; EP Publ. Nos. 2162538 and 2370577; and PCT Publ. No. WO 2015/074085.

In some embodiments, artificial nucleic acid analogs with backbone analogues include, but are not limited to, a bicyclic nucleotide analog such as locked nucleic acid (LNA), bridged nucleic acid (BNA), glycol nucleic acid (GNA), threose nucleic acid (TNA), and morpholino. The modified oligonucleotides that comprise these backbone analogs, although having a different backbone sugar, or in case of PNA, an amino acid residue in place of the ribose phosphate, still bind to RNA or DNA according to Watson and Crick pairing, but are immune to nuclease activity. LNAs are described, for example, in U.S. Pat. Nos. 6,268,490; 6,316,198; 6,403,566; 6,770,748; 6,998,484; 6,670,461; and 7,034,133; and PCT Publ. No. 1999/014226. Other suitable locked nucleotides that can be incorporated in the nucleic acid molecules encompassed by the present invention include those described in U.S. Pat. Nos. 6,403,566; 6,833,361; and 7,060,809. Other locked nucleic acid derivatives, such as D-oxy-LNA, α-L-oxy-LNA, β-D-amino-LNA, α-L-amino-LNA, thio-LNA, α-L-thio-LNA, seleno-LNA, methylene-LNA and β-D-ENA, can be incorporated into nucleic acid molecules encompassed by the present invention. Those LNA derivatives described in U.S. Pat. Nos. 7,569,575; 8,084,458; and 8,429,390, can also be incorporated into the nucleic acid molecules.

### Modified Sugars

In some embodiments, nucleic acid molecules encompassed by the present invention can comprise one or more sugar-modified nucleotides.

It can comprise about 1, about 2, about 3, about 4, about 5, about 6, about 7, about 8, about 9, about 10, or more sugar-modified nucleotides. Sugar-modified nucleotides useful in the invention include, but are not limited to: 2′-fluoro modified ribonucleotide, 2′-OMe modified ribonucleotide, 2′-deoxy ribonucleotide, 2′-amino modified ribonucleotide and 2′-thio modified ribonucleotide. The sugar-modified nucleotide can be, for example, 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine or 2′-amino-butyryl-pyrene-uridine. In addition to 2′ modification of the backbone sugar, the sugar group can be modified at other positions. The sugar group can comprise two different modifications at the same carbon of the sugar. The sugar group can also contain one or more carbons that possess the opposite stereochemical configuration than that of the corresponding carbon in ribose. Thus, a nucleic acid molecule can include nucleotides containing, e.g., arabinose, as the sugar. The nucleotide can have an alpha linkage at the 1′ position on the sugar, e.g., alpha-nucleosides. The nucleotide can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

The nucleic acid molecules encompassed by the present invention can also include abasic sugars, which lack a nucleobase at C-1′ or have other chemical groups in place of a nucleobase at Cr (see, e.g., U.S. Pat. No. 5,998,203). These abasic sugars can also be further containing modifications at one or more of the constituent sugar atoms. In other embodiments, nucleic acid molecules can also contain one or more sugars that are the L isomers. In one aspect, modification to the sugar group can also include replacement of the 4′-O with a sulfur, optionally substituted nitrogen or CH₂ group. In another aspect, modifications to the sugar group can also include acyclic nucleotides, wherein a C—C bond between ribose carbons is absent and/or at least one of ribose carbons or oxygen are independently or in combination absent from the nucleotide. Such acyclic nucleotides have been disclosed in U.S. Pat. Nos. 5,047,533 and 7,737,273, and U.S. Pat. Publ. No. 20130130378. It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration. The term “xylose configuration”, as used herein, refers to the placement of a substituent on the C3′ of ribose in the same configuration as the 3′-OH is in the xylose sugar. The hydrogen attached to C4′ and/or C1′ of the sugar group can be replaced by substitutes as described for 2′ modification. In one example, nucleic acid molecules encompassed by the present invention can comprise 2′-fluoro modified ribonucleotide. Preferably, the 2′-fluoro ribonucleotides are in the sense and antisense strands. More preferably, the 2′-fluoro ribonucleotides are every uridine and cytidine.

In some embodiments, the internucleoside linkage groups of the nucleic acid molecules encompassed by the present invention are modified.

The internucleoside linkage modification can be within the sense strand, antisense strand, or within the sense and antisense strands. The term “internucleoside linkage group” is intended to mean a group capable of covalently coupling together two nucleobases, such as between DNA residues, between RNA residues, between DNA and RNA residues and nucleotide analogues, between two non-LNA residues, between a non-LNA residue and a LNA residue, and between two LNA residues, etc. The naturally standard linkage is the phosphodiester linkage (PO linkage), consisting of —O—P(O)₂—O— (from 5′ to 3′ end), wherein the deoxyribose/ribose sugars are joined at both the 3′-hydroxyl and 5′-hydroxyl groups to phosphate groups in ester links, also known as “phosphodiester” bonds/linker. The linker can be modified by the replacement of one or both linking oxygens (i.e., oxygens that link the phosphate to the nucleoside), with nitrogen (bridged phosphoroamidates), sulfur (bridged phosphorothioates) and carbon (bridged methylenephosphonates). In some embodiments, the phosphate linker moiety can be replaced by non-phosphorus containing linkers, e.g., dephospho-linkers. While not wishing to be bound by theory, it is believed that since the charged phosphodiester group is the reaction center in nucleolytic degradation, its replacement with neutral structural mimics should impart enhanced nuclease stability. Examples of moieties which can replace the phosphate linker include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH₂ component parts.

In some embodiments, the modification of the linkage further comprises at least one of the oxygen atoms of one phosphate which is replaced or modified. In some aspects, one or both of the non-linking phosphate oxygens on the phosphate linker can be modified or replaced. The modified phosphates can include, but are not limited to, phosphonocarboxylate (in which one of the non-linking oxygen atoms has been replaced/modified with a carboxylic acid) (e.g., phosphoacetate, phosphonoformic acid, phosphoramidate); phosphorothioate (—O—P(O,S)—O—, —O—P(S)₂—O—); methylphosphonate (—O—P(OCH3)-O—), and alkyl or aryl phosphonates. As discussed herein, one or more atoms of the linkage between two successive monomers in the siRNA molecules encompassed by the present invention are modified. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═, —CH₂—CH₂—O—, —NR^{H}—CH₂—CH₂—, —CH₂—CH₂—NR^{H}, —CH₂—NR^{H}—CH₂, —O—CH₂—CH₂—NR^{H}—, —NR^{H}—CO—O—, —NR^{H}CO—NR^{H}—, —NR^{H}—CS—NR^{H}—, —NR^{H}—, —C(═NR^{H})—NR^{H}—, —NR^{H}CO—CH₂—NR^{H}—, —O—CO—O—, —O—CO—CH₂—O—, —O—CH₂—CO—O—, —CH₂—CO—NR^{H}—, —O—CO—NR^{H}—, —NR^{H}CO—CH₂—, —O—CH₂CO—NR^{H}—, —O—CH₂—CH₂—NR^{H}—, —CH═N—O—, —CH₂—NR^{H}O—, —CH₂—O—N═, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR″—CO—O—, —NR^{H}CO—NR^{H}—, —O—CO—O—, —O—CO—NR^{H}—, —NR^{H}CO—CH₂—, —O—CH₂—CO—NR^{H}, —O—CH₂—CH₂—NR^{H}—, —CO—NR^{H}—CH₂—, —CH₂—NR^{H}—CO—, —O—CH₂—CH₂—S—, —S—CH₂—CH₂—O—, —S—CH₂—CH₂—S—, —CH₂—SO₂—CH₂—, —CH₂—CO—NR^{H}—, —O—CH₂—CH₂—NR^{H}CO—, —CH₂—NCH₃—O—CH₂—, —S—CH₂—CH═, —O—PO(OCH₂CH₃)—O—, —O—PO(OCH₂CH₂S—R)—O—, —O—PO(BH₃)—O—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}S(O)₂—CH₂—, —O—S(O)₂—CH₂—, —O—P(O)₂—O—, —O—P(O,S)—O—, —O—P(S)₂—O—, —O—P(O,NR^{H})—O—, —O—PO(R″)—O—, —O—PO(CH₃)—O—, and —O—PO(NHR^{N})—O—, wherein R^{H} is selected from hydrogen and C₁₋₄-alkyl.

In the context encompassed by the present invention, preferred examples include phosphate, phosphodiester (PO) linkages and phosphorothioate (PS) linkages.

Phosphorodithioates have both non-bridging oxygens replaced by sulfur. The phosphorus center in the phosphorodithioates is achiral which precludes the formation of oligonucleotide diastereomers. Thus, while not wishing to be bound by theory, modifications to both non-linking oxygens, which eliminate the chiral center, e.g., phosphorodithioate formation, can be desirable in that they cannot produce diastereomer mixtures. Thus, the non-linking oxygens can be independently any one of O, S, Se, B, C, H, N, or OR (R is alkyl or aryl). In some embodiments, nucleic acid molecules encompassed by the present invention can contain one or more phosphorothioate linkages. For example, the polynucleotide can be partially phosphorothioate-linked, for example, phosphorothioate linkages can alternate with phosphodiester linkages. In certain embodiments, the oligonucleotide is fully phosphorothioate-linked. In other embodiments, the oligonucleotide has from one to seven, one to five or one to three phosphodiester linkages. Phosphorothioate linkages have been used to render oligonucleotides more resistant to nuclease cleavage. In addition to normal 5′-3′ linkage, modified oligonucleotide can have 5′-2′ linkage and those having inverted polarity wherein the adjacent pairs of nucleoside units are linked 3′-5′ to 5′-3′ or 2′-5′ to 5′-2′. Representative U.S. patents that teach modifications of internucleoside linkage groups include U.S. Pat. Nos. 5,519,126; 5,536,821; 5,541,306; 5,550,111; 5,563,253; 5,571,799; 5,587,361; 5,625,050; 5,378,825; 5,697,248 and 7,368,439. Other references that teach internucleoside linkage modifications include Mesmaeker et al. (1995) Curr. Opin. Struct. Biol. 5:343-355; Freier and Altmann (1997) Nucl. Acids Res. 25:4429-4443; and Micklefield (2001) Curr. Med. Chem. 8:1157-1179.

In some embodiments, nucleic acid molecules encompassed by the present invention can comprise one or more backbone-modified nucleotides.

The backbone-modified nucleotide is within the sense strand, antisense strand, or within the sense and antisense strands. A normal “backbone”, as used herein, refers to the repeating alternating sugar-phosphate sequences in a DNA or RNA molecule. In naturally occurring DNA and RNA molecules, the backbone of a nucleic acid molecule includes deoxyribose/ribose sugars joined at both the 3′-hydroxyl and 5′-hydroxyl groups to phosphate groups in ester links (i.e.PO linkage). The natural phosphodiester bonds can be replaced by amide bonds but the four atoms between two sugar units are kept. Such amide modifications can increase the thermodynamic stability of duplex formed with miRNA complement (see, e.g., Mesmaeker et al. (1997) Pure Appl. Chem. 3:437-440). In some embodiments, nucleic acid molecules encompassed by the present invention can contain chemical modifications with respect to non-locked nucleotides in the sequence, such as 2′ modification with respect to 2′hydroxyl. For example, incorporation of 2′-position modified nucleotides in an siRNA molecule can increase both resistance of the oligonucleotides to nucleases and their thermal stability with complementary targets. Various modifications at the 2′ positions can be independently selected from those that provide increased nuclease resistance, without compromising molecular interactions with the target or cellular machinery. Such modifications can be selected on the basis of their increased potency in vitro or in vivo. In some embodiments, the 2′ modification can be independently selected from a number of different “oxy” or “deoxy” substituents. Examples of “oxy”-2′ hydroxyl group modifications include alkoxy or aryloxy (e.g., Omethyl, R═H, alkyl, cycloalkyl, aryl, aralkyl, heteroaryl or sugar; polyethyleneglycols (PEG), O(CH₂CH₂O)ₙCH₂CH₂OR (n=1-50); O-AMINE or O—(CH₂)ₙAMINE (n=1-10), AMINE=NH₂; alkylamino, dialkylamino, heterocyclyl, arylamino, diaryl amino, heteroaryl amino, diheteroaryl amino, ethylene diamine or polyamino; and O—CH₂CH₂(NCH₂CH₂NMe₂)₂). “Deoxy” modifications include hydrogen (i.e., deoxyribose sugars, which are of particular relevance to the single-strand overhangs); halo (e.g., fluoro); amino (e.g., NH₂; alkylamino, dialkylamino, heterocyclyl, arylamino, diaryl amino, heteroaryl amino, diheteroaryl amino, or amino acid; NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino; —NHC(O)R(R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl.

Substantially all, or all, nucleotide 2′ positions of the non-locked nucleotides can be modified in certain embodiments. For example, the 2′ modifications can each be independently selected from O-methyl and fluoro. In exemplary embodiments, purine nucleotides each have a 2′ O-methyl and pyrrolidine nucleotides each have a 2′-F. In accordance with the present invention, 2′ position modifications can also include small hydrocarbon substituents. The hydrocarbon substituents include alkyl, alkenyl, alkynyl, and alkoxyalkyl, where the alkyl (including the alkyl portion of alkoxy), alkyl and alkyl can be substituted or unsubstituted. The alkyl, alkenyl, and alkynyl can be C1 to C10 alkyl, alkenyl or alkynyl, such as C1, C2, or C3. The hydrocarbon substituents can include one or two or three non-carbon atoms, which can be independently selected from N, O, and/or S. The 2′ modifications can further include the alkyl, alkenyl, and alkynyl as O-alkyl, O-alkenyl, and O-alkynyl. Exemplary 2′ modifications in accordance with the invention include 2′-H, 2′-O-alkyl (C1-3 alkyl, such as 2′O-Methyl or 2′OEt), 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethyiaminoethyioxyethyl (2′-O-DMAEOE), 2′-O—N-methylacetamido (2′-O-NMA) or gem 2′-OMe/2′F substitutions. In some embodiments, nucleic acid molecules encompassed by the present invention contains at least one 2′ position modified as 2′O-Methoxy (2′-OMe) in non-locked nucleotides. The oligonucleotide can contain from 1 to about 5 2′-O-Methoxy (2′-OMe) modified nucleotides, or from 1 to about 3 2′-O-Methoxy (2′-OMe) modified nucleotides. In some embodiments, all the nucleotides of the miR-124 mimic contain 2′-O-Methoxy (2′-OMe) modification. Other exemplary combinations of different types of 2′ position modifications can contain at least one 2′-halo modification (e.g., in place of a 2′ hydroxyl), such as 2′-fluoro, 2′-chloro, 2′-bromo, and 2′-iodo.

In some embodiments, the backbone of a strand or the strand of the nucleic acid molecule can be constructed wherein the phosphate linker and ribose sugar are replaced by nuclease resistant nucleosides or nucleotide surrogates. While not wishing to be bound by theory, it is believed that the absence of a repetitively charged backbone diminishes binding to proteins that recognize polyanions (e.g., nucleases). As non-limiting examples, such nucleotide surrogates include morpholino, cyclobutyl, pyrrolidine, peptide nucleic acid (PNA), aminoethylglycyl PNA (Aegina) and backbone-extended pyrimidine PNA (bepPNA) nucleoside surrogates (e.g., U.S. Pat. Nos. 5,359,044; 5,519,134; 5,142,047 and 5,235,033; Bioorganic & Medicinal Chemistry (1996), 4:5-23). A surrogate for the replacement of the sugar-phosphate backbone involves a PNA surrogate (peptide nucleic acid). The term “peptide nucleic acid (PNA)” is chemically synthesized polymer similar to DNA and RNA, wherein the backbone is composed of repeating N-(2-aminoethyl)-glycine (AEG) units linked by peptide bonds (Nielsen et al. (1991) Science 254:1497-1500). Synthetic oligonucleotides with PNAs have higher binding strength and greater specificity in binding to complementary DNAs or RNAs, with a PNA/DNA base mismatch being more desirable than a similar DNA/RNA duplex. PNAs are not easily recognized by either nucleases or proteases, making them resistant to enzyme degradation. PNAs are also stable over a wide pH range. PNA has been suggested for use in antisense and anti-gene therapy in a number of studies. PNA is resistant to DNases and proteases and can be further modified for increased cell penetration, etc.

### Other Modifications

Nucleic acid molecules encompassed by the present invention can also contain additional modifications, such as mismatches, bulges, or crosslinks. Similarly, they can also include other conjugates, such as linkers, heterofunctional cross linkers, dendrimer, nano-particle, peptides, organic compounds (e.g., fluorescent dyes), and/or photocleavable compounds. In some embodiments, nucleic acid molecules encompassed by the present invention can comprise any combination of two or more modifications as described herein. The nucleic acid sequences can comprise, independently, one or more modifications to one or more sugar moieties, to one or more internucleoside linkages, and/or to one or more nucleobases. As disclosed herein, these sequences can be modified with any combinations of chemical modifications.

In some embodiments, the nucleic acid molecule is a siRNA which comprises a nucleic acid sequence wherein the sense strand and anti-sense strand comprise one or more mismatches, for example, 1, 2, 3, 4, 5, 6, 7, 8, 9, 10, 11, 12, 13, 14, 15, 16, or more mismatches. The term “mismatch” refers to a basepair consisting of non-complementary bases, e.g., not normal complementary G:C, A:T or A:U base pairs. In some embodiments, the antisense strand of the siRNA molecule encompassed by the present invention and the target mRNA sequence can comprise one or more mismatches, for example, 1, 2, 3, 4, 5, 6, 7, 8, 9, 10, 11, 12, 13, 14, 15, 16, or more mismatches. In some instances, the mismatch can be downstream of the cleavage site referencing the antisense strand. More preferably, the mismatch can be present within 1-6 nucleotides from the 3′ end of the antisense strand. In another embodiment, the siRNA molecule encompassed by the present invention comprises a bulge, e.g., one or more, e.g., 1, 2, 3, 4, 5, 6, 7, 8, 9, 10, 11, 12, 13, 14, 15, 16, or more, unpaired bases in the duplex siRNA. Preferably, the bulge can be in the sense strand.

In some embodiments, the siRNA molecule encompassed by the present invention comprises one or more (e.g., about 1, 2, 3, 4, 5, 6, 7, 8, 9, 10, 11, 12, 13, 14, 15, 16, or more) crosslinks, e.g., a crosslink wherein the sense strand is crosslinked to the antisense strand of the siRNA duplex. Crosslinkers useful in the invention are those commonly known in the art, including, but not limited to, psoralen, mitomycin C, cisplatin, chloroethylnitrosoureas and the like. Preferably, the crosslink is present downstream of the cleavage site referencing the antisense strand, and more preferably, the crosslink is present at the 5′ end of the sense strand. In accordance with the present invention, siRNA derivatives are also included, such as a siRNA derivative having a single crosslink (e.g., a psoralen crosslink), a siRNA having a photocleavable biotin (e.g., photocleavable biotin), a peptide (e.g., a Tat peptide), a nanoparticle, a peptidomimetic, organic compounds (e.g., a dye such as a fluorescent dye), or dendrimer.

In some embodiments, nucleic acid molecules encompassed by the present invention can include other appended groups, such as peptides (e.g., for targeting host cell receptors in vivo), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. U.S.A. 84:648-652; PCT Pat. Publ. No. WO 88/09810) or the blood-brain barrier (see, e.g., PCT Publ. No. WO 89/10134). In addition, nucleic acid molecules can be modified with hybridization-triggered cleavage agents (see, e.g., Krol et al. (1988) BioTechniques 6:958-976) or intercalating agents (see, e.g., Zon (1988) Pharm. Res. 5:539-549).

In some embodiments, the siRNA molecules encompassed by the present invention can comprise any combinations of two or more modifications as described herein. The nucleic acid sequences set forth herein are independent of any modification to the nucleic acid. As such, nucleic acids defined by a SEQ ID NO can comprise, independently, one or more modifications to one or more sugar moieties, to one or more internucleoside linkages, and/or to one or more nucleobases. As disclosed herein, these sequences can be modified with any combinations of chemical modifications.

In some embodiments, the siRNA molecules encompassed by the present invention can include a sense strand and an antisense strand, wherein the antisense strand has a sequence sufficiently complementary to CCR2 mRNA sequence (SEQ ID NO: 1), or to CSF1R mRNA sequence (SEQ ID NO: 2), to direct target-specific RNA interference (RNAi) and wherein the sense strand and/or antisense strand is modified by the substitution of nucleotides with modified nucleotides. In one embodiment, the sense strand and/or antisense strand is modified by the substitution of at least one nucleotide. In another embodiment, the sense strand and/or antisense strand is modified by the substitution of at least 2 nucleotides, 3 nucleotides, 4 nucleotides, 5 nucleotides, 6 nucleotides, 7 nucleotides, 8 nucleotides 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, or more nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the nucleotides.

In some embodiments, the siRNA molecule that hybridize to CSF1R can comprise a modified sense nucleic acid sequence selected from the group consisting of nucleic acid sequences of SEQ ID NOs: 368 to 486 and a modified antisense nucleic acid sequence selected from the group consisting of nucleic acid sequences of SEQ ID NOs: 487 to 605 (Table 4). The target position of the sense and antisense duplex is indicated in the first column in Table 4. In other embodiments, a modified siRNA molecule that can result in a significant reduction of CSF1R mRNA in macrophages can be further modified to generate one or more variants. As non-limiting examples, some variants derived from siRNA molecules that hybridize to CSF1R can comprise a modified sense nucleic acid sequence selected from the group consisting of nucleic acid sequences of SEQ ID NOs: 883 to 921 and a modified antisense nucleic acid sequence selected from the group consisting of nucleic acid sequences of SEQ ID NOs: 922 and 960. These modifications can increase the efficiency, specificity and stability of the siRNA molecule.

TABLE 4
Modified sense and anti-sense sequences of siRNA specific to CSF1R

| | | | sense strand | SEQ | | antisense strand | SEQ |
|---|---|---|---|---|---|---|---|
| Duplex | duplex | | sequence | ID | | sequence | ID |
| Position | ID | SS ID | (5′-3′) | NO | AS ID | (5′-3′) | NO: |
| 346 | XD- | X28754 | ggGAAucccAGuGau | 368 | X28755 | CUCuAUcACUGG | 487 |
| | 08903 | | AGAGdTsdT | | | GAUUCCCdTsdT | |
| 410 | XD- | X28756 | uuGcGAuGuGuGGgc | 369 | X28757 | cAUUGCCcAcAcA | 488 |
| | 08904 | | AAuGdTsdT | | | UCGcAAdTsdT | |
| 412 | XD- | X28758 | gcGAuGuGuGGGca | 370 | X28759 | GCcAUUGCCcAcA | 489 |
| | 08905 | | AuGGcdTsdT | | | cAUCGCdTsdT | |
| 508 | XD- | X28760 | caAcGcuAccuuccAA | 371 | X28761 | GUUUUGGAAGG | 490 |
| | 08906 | | AAcdTsdT | | | uAGCGUUGdTsdT | |
| 510 | XD- | X28762 | acGcuAccuuccAaAA | 372 | X28763 | GUGUUUUGGAA | 491 |
| | 08907 | | cAcdTsdT | | | GGuAGCGUdTsdT | |
| 511 | XD- | X28764 | cgcuAccuuccAAaAc | 373 | X28765 | CGUGUUUUGGA | 492 |
| | 08908 | | AcGdTsdT | | | AGGuAGCGdTsdT | |
| 604 | XD- | X28766 | ugcccGGcccuGGaAc | 374 | X28767 | cACGUUCcAGGG | 493 |
| | 08909 | | GuGdTsdT | | | CCGGGcAdTsdT | |
| 675 | XD- | X28768 | ugcucAcAGAcccgGu | 375 | X28769 | AGcACCGGGUCU | 494 |
| | 08910 | | GcudTsdT | | | GUGAGcAdTsdT | |
| 700 | XD- | X28770 | agGcGucucGcuGguG | 376 | X28771 | ACGcACcAGCGA | 495 |
| | 08911 | | cGudTsdT | | | GACGCCUdTsdT | |
| 817 | XD- | X28772 | ucAAuGcAGuGcccu | 377 | X28773 | cAUcAGGGcACU | 496 |
| | 08912 | | GAuGdTsdT | | | GcAUUGAdTsdT | |
| 859 | XD- | X28774 | caGcAuccGGcuGaA | 378 | X28775 | cACUUUcAGCCG | 497 |
| | 08913 | | AGuGdTsdT | | | GAUGCUGdTsdT | |
| 921 | XD- | X28776 | caGAGcuGGuGcGg | 379 | X28777 | CGAAUCCGcACc | 498 |
| | 08914 | | AuucGdTsdT | | | AGCUCUGdTsdT | |
| 923 | XD- | X28778 | gaGcuGGuGcGGAuu | 380 | X28779 | CUCGAAUCCGcA | 499 |
| | 08915 | | cGAGdTsdT | | | CcAGCUCdTsdT | |
| 930 | XD- | X28780 | ugcGGAuucGAGGg | 381 | X28781 | GCCUCCCCUCGA | 500 |
| | 08916 | | GAGGcdTsdT | | | AUCCGcAdTsdT | |
| 948 | XD- | X28782 | cuGcccAGAucGugu | 382 | X28783 | GAGcAcACGAUC | 501 |
| | 08917 | | GcucdTsdT | | | UGGGcAGdTsdT | |
| 950 | XD- | X28784 | gcccAGAucGuGugcu | 383 | X28785 | CUGAGcAcACGA | 502 |
| | 08918 | | cAGdTsdT | | | UCUGGGCdTsdT | |
| 953 | XD- | X28786 | caGAucGuGuGcucA | 384 | X28787 | UGGCUGAGcAcA | 503 |
| | 08919 | | GccAdTsdT | | | CGAUCUGdTsdT | |
| 1053 | XD- | X28788 | auAAccGuuAccAaA | 385 | X28789 | ACUUUUUGGuAA | 504 |
| | 08920 | | AAGudTsdT | | | CGGUuAUdTsdT | |
| 1055 | XD- | X28790 | aaccGuuAccAAAaA | 386 | X28791 | GGACUUUUUGGu | 505 |
| | 08921 | | GuccdTsdT | | | AACGGUUdTsdT | |
| 1056 | XD- | X28792 | accGuuAccAAAAaG | 387 | X28793 | AGGACUUUUUG | 506 |
| | 08922 | | uccudTsdT | | | GuAACGGUdTsdT | |
| 1060 | XD- | X28794 | uuAccAAAAAGuccu | 388 | X28795 | GGUcAGGACUUU | 507 |
| | 08923 | | GAccdTsdT | | | UUGGuAAdTsdT | |
| 1288 | XD- | X28796 | agGuuuuAAcuGGacc | 389 | X28797 | GuAGGUCcAGUu | 508 |
| | 08924 | | uAcdTsdT | | | AAAACCUdTsdT | |
| 1289 | XD- | X28798 | gguuuuAAcuGGAccu | 390 | X28799 | GGuAGGUCcAGU | 509 |
| | 08925 | | AccdTsdT | | | uAAAACCdTsdT | |
| 1292 | XD- | X28800 | uuuAAcuGGAccuacc | 391 | X28801 | CcAGGuAGGUCc | 510 |
| | 08926 | | uGGdTsdT | | | AGUuAAAdTsdT | |
| 1293 | XD- | X28802 | uuAAcuGGAccuAcc | 392 | X28803 | CCcAGGuAGGUC | 511 |
| | 08927 | | uGGGdTsdT | | | cAGUuAAdTsdT | |
| 1300 | XD- | X28804 | gaccuAccuGGGAccc | 393 | X28805 | AAAGGGUCCcAG | 512 |
| | 08928 | | uuudTsdT | | | GuAGGUCdTsdT | |
| 1467 | XD- | X28806 | ugAcGuuuGAGcucA | 394 | X28807 | AGGGUGAGCUcA | 513 |
| | 08929 | | cccudTsdT | | | AACGUcAdTsdT | |
| 1468 | XD- | X28808 | gacGuuuGAGcucaccc | 395 | X28809 | AAGGGUGAGCUc | 514 |
| | 08930 | | uudTsdT | | | AAACGUCdTsdT | |
| 1473 | XD- | X28810 | uuGAGcucAcccuucG | 396 | X28811 | uAUCGAAGGGUG | 515 |
| | 08931 | | AuAdTsdT | | | AGCUcAAdTsdT | |
| 1476 | XD- | X28812 | agcucAcccuucGauAc | 397 | X28813 | GGGuAUCGAAGG | 516 |
| | 08932 | | ccdTsdT | | | GUGAGCUdTsdT | |
| 1485 | XD- | X28814 | uucGAuAccccccaGA | 398 | X28815 | ACCUCUGGGGG | 517 |
| | 08933 | | GGudTsdT | | | GuAUCGAAdTsdT | |
| 1488 | XD- | X28816 | gauAccccccAGAgGu | 399 | X28817 | CUuACCUCUGGG | 518 |
| | 08934 | | AAGdTsdT | | | GGGuAUCdTsdT | |
| 1715 | XD- | X28818 | gaGAccuuAGAGcac | 400 | X28819 | GGUUGUGCUCuA | 519 |
| | 08935 | | AAccdTsdT | | | AGGUCUCdTsdT | |
| 1717 | XD- | X28820 | gaccuuAGAGcAcaAc | 401 | X28821 | UUGGUUGUGCU | 520 |
| | 08936 | | cAAdTsdT | | | CuAAGGUCdTsdT | |
| 1759 | XD- | X28822 | caGcGuGGGGAGug | 402 | X28823 | GGAGCcACUCCC | 521 |
| | 08937 | | GcuccdTsdT | | | cACGCUGdTsdT | |
| 1815 | XD- | X28824 | aucccccGGAuGAguu | 403 | X28825 | AGGAACUcAUCC | 522 |
| | 08938 | | ccudTsdT | | | GGGGGAUdTsdT | |
| 1819 | XD- | X28826 | cccGGAuGAGuuccuc | 404 | X28827 | GAAGAGGAACUc | 523 |
| | 08939 | | uucdTsdT | | | AUCCGGGdTsdT | |
| 1847 | XD- | X28828 | guGGucGccuGcAuG | 405 | X28829 | UGGAcAUGcAGG | 524 |
| | 08940 | | uccAdTsdT | | | CGACcACdTsdT | |
| 1896 | XD- | X28830 | ugcuAuuGuAcAAgu | 406 | X28831 | UuAuACUUGuAcA | 525 |
| | 08941 | | AuAAdTsdT | | | AuAGcAdTsdT | |
| 1931 | XD- | X28832 | caGGuccGcuGGAaG | 407 | X28833 | UGAUCUUCcAGC | 526 |
| | 08942 | | AucAdTsdT | | | GGACCUGdTsdT | |
| 1933 | XD- | X28834 | gguccGcuGGAAGau | 408 | X28835 | GAUGAUCUUCcA | 527 |
| | 08943 | | cAucdTsdT | | | GCGGACCdTsdT | |
| 1935 | XD- | X28836 | uccGcuGGAAGAuc | 409 | X28837 | UCGAUGAUCUU | 528 |
| | 08944 | | AucGAdTsdT | | | CcAGCGGAdTsdT | |
| 1936 | XD- | X28838 | cc GcuGGAAGAucau | 410 | X28839 | CUCGAUGAUCU | 529 |
| | 08945 | | cGAGdTsdT | | | UCcAGCGGdTsdT | |
| 1946 | XD- | X28840 | aucAucGAGAGcuau | 411 | X28841 | CCUcAuAGCUCU | 530 |
| | 08946 | | GAGGdTsdT | | | CGAUGAUdTsdT | |
| 1948 | XD- | X28842 | caucGAGAGcuAugA | 412 | X28843 | GCCCUcAuAGCU | 531 |
| | 08947 | | GGGcdTsdT | | | CUCGAUGdTsdT | |
| 1958 | XD- | X28844 | uauGAGGGcAAcAg | 413 | X28845 | uAuAACUGUUGC | 532 |
| | 08948 | | uuAuAdTsdT | | | CCUcAuAdTsdT | |
| 1962 | XD- | X28192 | agGGcAAcAGuuAu | 414 | X28193 | AAAGuAuAACUG | 533 |
| | 08718 | | AcuuudTsdT | | | UUGCCCUdTsdT | |
| 1964 | XD- | X28846 | ggcAAcAGuuAuAcu | 415 | X28847 | UGAAAGuAuAAC | 534 |
| | 08950 | | uucAdTsdT | | | UGUUGCCdTsdT | |
| 1990 | XD- | X28848 | cacGcAGcuGccuuAc | 416 | X28849 | GUUGuAAGGcAG | 535 |
| | 08951 | | AAcdTsdT | | | CUGCGUGdTsdT | |
| 2021 | XD- | X28850 | uucccccGGAAcAacc | 417 | X28851 | GcAGGUUGUUCC | 536 |
| | 08952 | | uGcdTsdT | | | GGGGGAAdTsdT | |
| 2026 | XD- | X28852 | ccGGAAcAAccuGcA | 418 | X28853 | AAACUGcAGGUU | 537 |
| | 08953 | | GuuudTsdT | | | GUUCCGGdTsdT | |
| 2042 | XD- | X28854 | uuuGGuAAGAcccuc | 419 | X28855 | CUCCGAGGGUC | 538 |
| | 08954 | | GGAGdTsdT | | | UuACcAAAdTsdT | |
| 2044 | XD- | X28856 | ugGuAAGAcccucgG | 420 | X28857 | AGCUCCGAGGG | 539 |
| | 08955 | | AGcudTsdT | | | UCUuACcAdTsdT | |
| 2145 | XD- | X28858 | ugAAGuccAcGGccc | 421 | X28859 | GcAUGGGCCGUG | 540 |
| | 08956 | | AuGcdTsdT | | | GACUUcAdTsdT | |
| 2146 | XD- | X28860 | gaAGuccAcGGcccAu | 422 | X28861 | AGcAUGGGCCGU | 541 |
| | 08957 | | GcudTsdT | | | GGACUUCdTsdT | |
| 2264 | XD- | X28862 | ccuGuAcuGGucAuc | 423 | X28863 | CCGUGAUGACcA | 542 |
| | 08958 | | AcGGdTsdT | | | GuAcAGGdTsdT | |
| 2265 | XD- | X28864 | cuGuAcuGGucAucA | 424 | X28865 | UCCGUGAUGACc | 543 |
| | 08959 | | cGGAdTsdT | | | AGuAcAGdTsdT | |
| 2266 | XD- | X28866 | uguAcuGGucAucacG | 425 | X28867 | CUCCGUGAUGA | 544 |
| | 08960 | | GAGdTsdT | | | CcAGuAcAdTsdT | |
| 2268 | XD- | X28868 | uacuGGucAucAcgG | 426 | X28869 | uACUCCGUGAUG | 545 |
| | 08961 | | AGuAdTsdT | | | ACcAGuAdTsdT | |
| 2272 | XD- | X28870 | ggucAucAcGGAGuA | 427 | X28871 | AcAGuACUCCGU | 546 |
| | 08962 | | cuGudTsdT | | | GAUGACCdTsdT | |
| 2276 | XD- | X28872 | aucAcGGAGuAcugu | 428 | X28873 | AGcAAcAGuACU | 547 |
| | 08963 | | uGcudTsdT | | | CCGUGAUdTsdT | |
| 2277 | XD- | X28874 | ucAcGGAGuAcuGuu | 429 | X28875 | uAGcAAcAGuACU | 548 |
| | 08964 | | GcuAdTsdT | | | CCGUGAdTsdT | |
| 2279 | XD- | X28876 | acGGAGuAcuGuugc | 430 | X28877 | cAuAGcAAcAGuA | 549 |
| | 08965 | | uAuGdTsdT | | | CUCCGUdTsdT | |
| 2283 | XD- | X28878 | aguAcuGuuGcuAuG | 431 | X28879 | UCGCcAuAGcAAc | 550 |
| | 08966 | | GcGAdTsdT | | | AGuACUdTsdT | |
| 2288 | XD- | X28880 | uguuGcuAuGGcGacc | 432 | X28881 | GcAGGUCGCcAu | 551 |
| | 08967 | | uGcdTsdT | | | AGcAAcAdTsdT | |
| 2292 | XD- | X28882 | gcuAuGGcGAccugcu | 433 | X28883 | UUGAGcAGGUCG | 552 |
| | 08968 | | cAAdTsdT | | | CcAuAGCdTsdT | |
| 2295 | XD- | X28884 | auGGcGAccuGcucA | 434 | X28885 | AAGUUGAGcAGG | 553 |
| | 08969 | | AcuudTsdT | | | UCGCcAUdTsdT | |
| 2305 | XD- | X28886 | gcucAAcuuucuGcGA | 435 | X28887 | CCUUCGcAGAAA | 554 |
| | 08970 | | AGGdTsdT | | | GUUGAGCdTsdT | |
| 2308 | XD- | X28888 | caAcuuucuGcGAaGG | 436 | X28889 | CUUCCUUCGcAG | 555 |
| | 08971 | | AAGdTsdT | | | AAAGUUGdTsdT | |
| 2313 | XD- | X28890 | uucuGcGAAGGAAg | 437 | X28891 | UcAGCCUUCCUU | 556 |
| | 08972 | | GcuGAdTsdT | | | CGcAGAAdTsdT | |
| 2444 | XD- | X28892 | caGGGuGuGGAcAcc | 438 | X28893 | cAuAGGUGUCcAc | 557 |
| | 08973 | | uAuGdTsdT | | | ACCCUGdTsdT | |
| 2518 | XD- | X28894 | ggAcAAGGAGGAug | 439 | X28895 | CCGUCcAUCCUC | 558 |
| | 08974 | | GAcGGdTsdT | | | CUUGUCCdTsdT | |
| 2600 | XD- | X28896 | gcuuccAAGAAuugc | 440 | X28897 | GGAUGcAAUUCU | 559 |
| | 08975 | | AuccdTsdT | | | UGGAAGCdTsdT | |
| 2679 | XD- | X28898 | acuucGGGcuGGcuA | 441 | X28899 | UCCCuAGCcAGC | 560 |
| | 08976 | | GGGAdTsdT | | | CCGAAGUdTsdT | |
| 2687 | XD- | X28900 | cuGGcuAGGGAcAu | 442 | X28901 | UcAUGAUGUCCC | 561 |
| | 08977 | | cAuGAdTsdT | | | uAGCcAGdTsdT | |
| 2689 | XD- | X28902 | ggcuAGGGAcAucau | 443 | X28903 | AUUcAUGAUGUC | 562 |
| | 08978 | | GAAudTsdT | | | CCuAGCCdTsdT | |
| 2774 | XD- | X28904 | uuuGAcuGuGucuacA | 444 | X28905 | CCGUGuAGAcAc | 563 |
| | 08979 | | cGGdTsdT | | | AGUcAAAdTsdT | |
| 2776 | XD- | X28906 | ugAcuGuGucuAcacG | 445 | X28907 | AACCGUGuAGAc | 564 |
| | 08980 | | GuudTsdT | | | AcAGUcAdTsdT | |
| 2778 | XD- | X28908 | acuGuGucuAcAcgGu | 446 | X28909 | UGAACCGUGuAG | 565 |
| | 08981 | | ucAdTsdT | | | AcAcAGUdTsdT | |
| 2781 | XD- | X28910 | guGucuAcAcGGuuc | 447 | X28911 | CUCUGAACCGU | 566 |
| | 08982 | | AGAGdTsdT | | | GuAGAcACdTsdT | |
| 2785 | XD- | X28912 | cuAcAcGGuucAGaG | 448 | X28913 | GUCGCUCUGAA | 567 |
| | 08983 | | cGAcdTsdT | | | CCGUGuAGdTsdT | |
| 2788 | XD- | X28914 | cacGGuucAGAGcgA | 449 | X28915 | GACGUCGCUCU | 568 |
| | 08984 | | cGucdTsdT | | | GAACCGUGdTsdT | |
| 2791 | XD- | X28916 | gguucAGAGcGAcgu | 450 | X28917 | CcAGACGUCGCU | 569 |
| | 08985 | | cuGGdTsdT | | | CUGAACCdTsdT | |
| 2793 | XD- | X28918 | uucAGAGcGAcGucu | 451 | X28919 | GACcAGACGUCG | 570 |
| | 08986 | | GGucdTsdT | | | CUCUGAAdTsdT | |
| 2843 | XD- | X28920 | cuuGGGcuGAAuccc | 452 | X28921 | GGuAGGGAUUcA | 571 |
| | 08987 | | uAccdTsdT | | | GCCcAAGdTsdT | |
| 2874 | XD- | X28922 | ugAAcAGcAAGuucu | 453 | X28923 | UuAuAGAACUUG | 572 |
| | 08988 | | AuAAdTsdT | | | CUGUUcAdTsdT | |
| 2880 | XD- | X28924 | gcAAGuucuAuAAac | 454 | X28925 | ACcAGUUuAuAG | 573 |
| | 08989 | | uGGudTsdT | | | AACUUGCdTsdT | |
| 2940 | XD- | X28926 | agAAuAuAuAcAGc | 455 | X28927 | AUGAUGCUGuAu | 574 |
| | 08990 | | AucAudTsdT | | | AuAUUCUdTsdT | |
| 3047 | XD- | X28928 | agAGAGcGGGAcua | 456 | X28929 | UGGuAuAGUCCC | 575 |
| | 08991 | | uAccAdTsdT | | | GCUCUCUdTsdT | |
| 3048 | XD- | X28930 | gaGAGcGGGAcuAu | 457 | X28931 | UUGGuAuAGUCC | 576 |
| | 08992 | | AccAAdTsdT | | | CGCUCUCdTsdT | |
| 3049 | XD- | X28932 | agAGcGGGAcuAuac | 458 | X28933 | AUUGGuAuAGUC | 577 |
| | 08993 | | cAAudTsdT | | | CCGCUCUdTsdT | |
| 3052 | XD- | X28934 | gcGGGAcuAuAccaA | 459 | X28935 | cAGAUUGGuAuA | 578 |
| | 08994 | | ucuGdTsdT | | | GUCCCGCdTsdT | |
| 3055 | XD- | X28936 | ggAcuAuAccAAucu | 460 | X28937 | CGGcAGAUUGGu | 579 |
| | 08995 | | GccGdTsdT | | | AuAGUCCdTsdT | |
| 3149 | XD- | X28938 | ugcuGcGAGcAAGg | 461 | X28939 | uAUCCCCUUGCU | 580 |
| | 08996 | | GGAuAdTsdT | | | CGcAGcAdTsdT | |
| 3153 | XD- | X28940 | gcGAGcAAGGGGA | 462 | X28941 | GCGAuAUCCCCU | 581 |
| | 08997 | | uAucGcdTsdT | | | UGCUCGCdTsdT | |
| 3154 | XD- | X28942 | cgAGcAAGGGGAua | 463 | X28943 | GGCGAuAUCCCC | 582 |
| | 08998 | | ucGccdTsdT | | | UUGCUCGdTsdT | |
| 3164 | XD- | X28944 | gauAucGcccAGcccuu | 464 | X28945 | GcAAGGGCUGGG | 583 |
| | 08999 | | GcdTsdT | | | CGAuAUCdTsdT | |
| 3186 | XD- | X28946 | agcccAAcAAcuAucA | 465 | X28947 | AACUGAuAGUUG | 584 |
| | 09000 | | GuudTsdT | | | UUGGGCUdTsdT | |
| 3187 | XD- | X28948 | gcccAAcAAcuAucA | 466 | X28949 | GAACUGAuAGUU | 585 |
| | 09001 | | GuucdTsdT | | | GUUGGGCdTsdT | |
| 3188 | XD- | X28950 | cccAAcAAcuAucaGu | 467 | X28951 | AGAACUGAuAGU | 586 |
| | 09002 | | ucudTsdT | | | UGUUGGGdTsdT | |
| 3292 | XD- | X28952 | acAAAcucuGccuucG | 468 | X28953 | GACCGAAGGcAG | 587 |
| | 09003 | | GucdTsdT | | | AGUUUGUdTsdT | |
| 3298 | XD- | X28954 | ucuGccuucGGucauuu | 469 | X28955 | UGAAAUGACCG | 588 |
| | 09004 | | cAdTsdT | | | AAGGcAGAdTsdT | |
| 3301 | XD- | X28956 | gccuucGGucAuuucA | 470 | X28957 | GAGUGAAAUGA | 589 |
| | 09005 | | cucdTsdT | | | CCGAAGGCdTsdT | |
| 3305 | XD- | X28958 | ucGGucAuuucAcucA | 471 | X28959 | UGUUGAGUGAA | 590 |
| | 09006 | | AcAdTsdT | | | AUGACCGAdTsdT | |
| 3457 | XD- | X28960 | cucAuGGuGuuGGcc | 472 | X28961 | ACGAGGCcAAcA | 591 |
| | 09007 | | ucGudTsdT | | | CcAUGAGdTsdT | |
| 3458 | XD- | X28962 | ucAuGGuGuuGGccu | 473 | X28963 | cACGAGGCcAAc | 592 |
| | 09008 | | cGuGdTsdT | | | ACcAUGAdTsdT | |
| 3459 | XD- | X28964 | cauGGuGuuGGccuc | 474 | X28965 | AcACGAGGCcAA | 593 |
| | 09009 | | GuGudTsdT | | | cACcAUGdTsdT | |
| 3462 | XD- | X28966 | gguGuuGGccucGuGu | 475 | X28967 | cAAAcACGAGGC | 594 |
| | 09010 | | uuGdTsdT | | | cAAcACCdTsdT | |
| 3473 | XD- | X28968 | cguGuuuGcuAuGccA | 476 | X28969 | AGUUGGcAuAGc | 595 |
| | 09011 | | AcudTsdT | | | AAAcACGdTsdT | |
| 3808 | XD- | X28970 | agcuAAAAGuuGGg | 477 | X28971 | AcACCCCcAACU | 596 |
| | 09012 | | GGuGudTsdT | | | UUuAGCUdTsdT | |
| 3911 | XD- | X28972 | ccAAGcuGAcucAucc | 478 | X28973 | UuAGGAUGAGUc | 597 |
| | 09013 | | uAAdTsdT | | | AGCUUGGdTsdT | |
| 3914 | XD- | X28974 | agcuGAcucAuccuAA | 479 | X28975 | uAGUuAGGAUGA | 598 |
| | 09014 | | cuAdTsdT | | | GUcAGCUdTsdT | |
| 3916 | XD- | X28976 | cuGAcucAuccuAacu | 480 | X28977 | GUuAGUuAGGAU | 599 |
| | 09015 | | AAcdTsdT | | | GAGUcAGdTsdT | |
| 3917 | XD- | X28978 | ugAcucAuccuAAcuA | 481 | X28979 | UGUuAGUuAGGA | 600 |
| | 09016 | | AcAdTsdT | | | UGAGUcAdTsdT | |
| 3918 | XD- | X28980 | gacucAuccuAAcuAA | 482 | X28981 | CUGUuAGUuAGG | 601 |
| | 09017 | | cAGdTsdT | | | AUGAGUCdTsdT | |
| 3924 | XD- | X28982 | uccuAAcuAAcAGuc | 483 | X28983 | GCGUGACUGUuA | 602 |
| | 09018 | | AcGcdTsdT | | | GUuAGGAdTsdT | |
| 3925 | XD- | X28984 | ccuAAcuAAcAGucA | 484 | X28985 | GGCGUGACUGUu | 603 |
| | 09019 | | cGccdTsdT | | | AGUuAGGdTsdT | |
| 3958 | XD- | X28986 | uccAcAuuAAAcuaAc | 485 | X28987 | GCUGUuAGUUuA | 604 |
| | 09020 | | AGcdTsdT | | | AUGUGGAdTsdT | |
| 3959 | XD- | X28988 | ccAcAuuAAAcuAac | 486 | X28989 | UGCUGUuAGUUu | 605 |
| | 09021 | | AGcAdTsdT | | | AAUGUGGdTsdT | |

In some embodiments, the siRNA molecule that hybridize to CCR2 can comprise a modified sense nucleic acid sequence selected from the group consisting of nucleic acid sequences of SEQ ID NOs: 606 to 743 and a modified antisense nucleic acid sequence selected from the group consisting of nucleic acid sequences of SEQ ID NOs: 744 to 881 (Table 5). The identifier of the sense and antisense duplex is indicated in the first column in Table 5. In other embodiments, a modified siRNA molecule that can result in a significant reduction of CCR2 mRNA in macrophages can be further modified to generate one or more variants. As non-limiting examples, some variants derived from siRNA molecules that hybridize to CCR2 can comprise a modified sense nucleic acid sequence selected from the group consisting of nucleic acid sequences of SEQ ID NOs: 961 to 1001 and a modified antisense nucleic acid sequence selected from the group consisting of nucleic acid sequences of SEQ ID NOs: 1002 and 1042. These modifications can increase the efficiency, specificity and stability of the siRNA molecule.

TABLE 5
Modified sense and anti-sense sequences of siRNA specific to CCR2

| | | | SEQ | | | SEQ |
|---|---|---|---|---|---|---|
| Duplex | SS | Sense Sequence | ID | AS | Antisense Sequence | ID |
| ID | ID | (5′-3′) | NO | ID | (5′-3′) | NO |
| XD-09027 | X29012 | caAGGAcGcAuuucccc | 606 | X29013 | CUGGGGAAAUGCGUCC | 744 |
| | | AGdTsdT | | | UUGdTsdT | |
| XD-09028 | X29014 | aaGGAcGcAuuuccccA | 607 | X29015 | ACUGGGGAAAUGCGUC | 745 |
| | | GudTsdT | | | CUUdTsdT | |
| XD-09029 | X29016 | ggAcGcAuuuccccAGu | 608 | X29017 | GuACUGGGGAAAUGCG | 746 |
| | | AcdTsdT | | | UCCdTsdT | |
| XD-09030 | X29018 | gacGcAuuuccccaGuAc | 609 | X29019 | UGuACUGGGGAAAUGC | 747 |
| | | AdTsdT | | | GUCdTsdT | |
| XD-09031 | X29020 | acGcAuuuccccAguAcA | 610 | X29021 | AUGuACUGGGGAAAUG | 748 |
| | | udTsdT | | | CGUdTsdT | |
| XD-09032 | X29022 | cgcAuuuccccAGuAcAu | 611 | X29023 | GAUGuACUGGGGAAAU | 749 |
| | | cdTsdT | | | GCGdTsdT | |
| XD-09033 | X29024 | cauuuccccAGuAcAucc | 612 | X29025 | UGGAUGuACUGGGGAA | 750 |
| | | AdTsdT | | | AUGdTsdT | |
| XD-09034 | X29026 | auuuccccAGuAcauccA | 613 | X29027 | GUGGAUGuACUGGGGA | 751 |
| | | cdTsdT | | | AAUdTsdT | |
| XD-09035 | X29028 | uuuccccAGuAcAuccAc | 614 | X29029 | UGUGGAUGuACUGGGG | 752 |
| | | AdTsdT | | | AAAdTsdT | |
| XD-09036 | X29030 | uccccAGuAcAuccAcA | 615 | X29031 | GUUGUGGAUGuACUGG | 753 |
| | | AcdTsdT | | | GGAdTsdT | |
| XD-09037 | X29032 | caGuAcAuccAcAacAu | 616 | X29033 | GcAUGUUGUGGAUGuA | 754 |
| | | GcdTsdT | | | CUGdTsdT | |
| XD-09038 | X29034 | guAcAuccAcAAcauGc | 617 | X29035 | cAGcAUGUUGUGGAUGu | 755 |
| | | uGdTsdT | | | ACdTsdT | |
| XD-09039 | X29036 | acAuccAcAAcAugcuG | 618 | X29037 | GAcAGcAUGUUGUGGA | 756 |
| | | ucdTsdT | | | UGUdTsdT | |
| XD-09040 | X29038 | auGcuGuccAcAucucGu | 619 | X29039 | AACGAGAUGUGGAcAG | 757 |
| | | udTsdT | | | cAUdTsdT | |
| XD-09041 | X29040 | ugcuGuccAcAucucGuu | 620 | X29041 | GAACGAGAUGUGGAcA | 758 |
| | | cdTsdT | | | GcAdTsdT | |
| XD-09042 | X29042 | gcuGuccAcAucucGuuc | 621 | X29043 | AGAACGAGAUGUGGAc | 759 |
| | | udTsdT | | | AGCdTsdT | |
| XD-09043 | X29044 | cuGuccAcAucucguucuc | 622 | X29045 | GAGAACGAGAUGUGGA | 760 |
| | | dTsdT | | | cAGdTsdT | |
| XD-09044 | X29046 | guccAcAucucGuucucG | 623 | X29047 | CCGAGAACGAGAUGUG | 761 |
| | | GdTsdT | | | GACdTsdT | |
| XD-09045 | X29048 | uccAcAucucGuucucGG | 624 | X29049 | ACCGAGAACGAGAUGU | 762 |
| | | udTsdT | | | GGAdTsdT | |
| XD-09046 | X29050 | ccAcAucucGuucucGGu | 625 | X29051 | AACCGAGAACGAGAUG | 763 |
| | | udTsdT | | | UGGdTsdT | |
| XD-09047 | X29052 | cacAucucGuucucGGuu | 626 | X29053 | AAACCGAGAACGAGAU | 764 |
| | | udTsdT | | | GUGdTsdT | |
| XD-09048 | X29054 | acAucucGuucucgGuuu | 627 | X29055 | uAAACCGAGAACGAGA | 765 |
| | | AdTsdT | | | UGUdTsdT | |
| XD-09049 | X29056 | caucucGuucucGguuuA | 628 | X29057 | AuAAACCGAGAACGAG | 766 |
| | | udTsdT | | | AUGdTsdT | |
| XD-09050 | X29058 | ucucGuucucGGuuuAuc | 629 | X29059 | UGAuAAACCGAGAACG | 767 |
| | | AdTsdT | | | AGAdTsdT | |
| XD-09051 | X29060 | cucGuucucGGuuuAuc | 630 | X29061 | CUGAuAAACCGAGAAC | 768 |
| | | AGdTsdT | | | GAGdTsdT | |
| XD-09052 | X29062 | ucGuucucGGuuuaucA | 631 | X29063 | UCUGAuAAACCGAGAA | 769 |
| | | GAdTsdT | | | CGAdTsdT | |
| XD-09053 | X29064 | cguucucGGuuuAucAG | 632 | X29065 | UUCUGAuAAACCGAGA | 770 |
| | | AAdTsdT | | | ACGdTsdT | |
| XD-09054 | X29066 | uucucGGuuuAucaGAA | 633 | X29067 | AUUUCUGAuAAACCGA | 771 |
| | | AudTsdT | | | GAAdTsdT | |
| XD-09055 | X29068 | ucucGGuuuAucAgAA | 634 | X29069 | uAUUUCUGAuAAACCG | 772 |
| | | AuAdTsdT | | | AGAdTsdT | |
| XD-09056 | X29070 | cucGGuuuAucAGaAA | 635 | X29071 | GuAUUUCUGAuAAACC | 773 |
| | | uAcdTsdT | | | GAGdTsdT | |
| XD-09057 | X29072 | ucGGuuuAucAGAaAu | 636 | X29073 | GGuAUUUCUGAuAAAC | 774 |
| | | AccdTsdT | | | CGAdTsdT | |
| XD-09058 | X29074 | uuuAucAGAAAuAccA | 637 | X29075 | CGUUGGuAUUUCUGAu | 775 |
| | | AcGdTsdT | | | AAAdTsdT | |
| XD-09059 | X29076 | agAGcGGuGAAGAaG | 638 | X29077 | GUGACUUCUUcACCGC | 776 |
| | | ucAcdTsdT | | | UCUdTsdT | |
| XD-09060 | X29078 | cgGuGAAGAAGucacc | 639 | X29079 | GGUGGUGACUUCUUcA | 777 |
| | | AccdTsdT | | | CCGdTsdT | |
| XD-09061 | X29080 | aaGucAccAccuuuuuuG | 640 | X29081 | UcAAAAAAGGUGGUGA | 778 |
| | | AdTsdT | | | CUUdTsdT | |
| XD-09062 | X29082 | ucAccAccuuuuuuGAuu | 641 | X29083 | uAAUcAAAAAAGGUGG | 779 |
| | | AdTsdT | | | UGAdTsdT | |
| XD-09063 | X29084 | caccAccuuuuuugAuuA | 642 | X29085 | AuAAUcAAAAAAGGUG | 780 |
| | | udTsdT | | | GUGdTsdT | |
| XD-09064 | X29086 | accAccuuuuuuGauuAu | 643 | X29087 | cAuAAUcAAAAAAGGUG | 781 |
| | | GdTsdT | | | GUdTsdT | |
| XD-09065 | X29088 | uuuuGAuuAuGAuuAc | 644 | X29089 | ACCGuAAUcAuAAUcAA | 782 |
| | | GGudTsdT | | | AAdTsdT | |
| XD-09066 | X29090 | ugAuuAuGAuuAcgGu | 645 | X29091 | AGcACCGuAAUcAuAAU | 783 |
| | | GcudTsdT | | | cAdTsdT | |
| XD-09067 | X29092 | uuAuGAuuAcGGugcuc | 646 | X29093 | GGGAGcACCGuAAUcAu | 784 |
| | | cdTsdT | | | AAdTsdT | |
| XD-09068 | X29094 | uauGAuuAcGGuGcucc | 647 | X29095 | AGGGAGcACCGuAAUcA | 785 |
| | | cudTsdT | | | uAdTsdT | |
| XD-09069 | X29096 | ugAuuAcGGuGcucccu | 648 | X29097 | AcAGGGAGcACCGuAAU | 786 |
| | | GudTsdT | | | cAdTsdT | |
| XD-09070 | X29098 | gauuAcGGuGcucccuG | 649 | X29099 | GAcAGGGAGcACCGuAA | 787 |
| | | ucdTsdT | | | UCdTsdT | |
| XD-09071 | X29100 | auuAcGGuGcucccuGuc | 650 | X29101 | UGAcAGGGAGcACCGuA | 788 |
| | | AdTsdT | | | AUdTsdT | |
| XD-09072 | X29102 | uuAcGGuGcucccuGuc | 651 | X29103 | AUGAcAGGGAGcACCGu | 789 |
| | | AudTsdT | | | AAdTsdT | |
| XD-09073 | X29104 | acGGuGcucccuGucAu | 652 | X29105 | UuAUGAcAGGGAGcACC | 790 |
| | | AAdTsdT | | | GUdTsdT | |
| XD-09074 | X29106 | guGcucccuGucAuAAA | 653 | X29107 | AAUUuAUGAcAGGGAGc | 791 |
| | | uudTsdT | | | ACdTsdT | |
| XD-09075 | X29108 | ugcucccuGucAuaAAuu | 654 | X29109 | AAAUUuAUGAcAGGGA | 792 |
| | | udTsdT | | | GcAdTsdT | |
| XD-09076 | X29110 | cucccuGucAuAAauuu | 655 | X29111 | UcAAAUUuAUGAcAGGG | 793 |
| | | GAdTsdT | | | AGdTsdT | |
| XD-09077 | X29112 | ccuGucAuAAAuuuGA | 656 | X29113 | ACGUcAAAUUuAUGAcA | 794 |
| | | cGudTsdT | | | GGdTsdT | |
| XD-09078 | X29114 | ugucAuAAAuuuGacGu | 657 | X29115 | UcACGUcAAAUUuAUGA | 795 |
| | | GAdTsdT | | | cAdTsdT | |
| XD-09079 | X29116 | gucAuAAAuuuGAcGu | 658 | X29117 | UUcACGUcAAAUUuAUG | 796 |
| | | GAAdTsdT | | | ACdTsdT | |
| XD-09080 | X29118 | ucAuAAAuuuGAcguG | 659 | X29119 | CUUcACGUcAAAUUuAU | 797 |
| | | AAGdTsdT | | | GAdTsdT | |
| XD-09081 | X29120 | auAAAuuuGAcGugAA | 660 | X29121 | UGCUUcACGUcAAAUUu | 798 |
| | | GcAdTsdT | | | AUdTsdT | |
| XD-09082 | X29122 | aauuuGAcGuGAAgcA | 661 | X29123 | AUUUGCUUcACGUcAA | 799 |
| | | AAudTsdT | | | AUUdTsdT | |
| XD-09083 | X29124 | auuuGAcGuGAAGcA | 662 | X29125 | AAUUUGCUUcACGUcA | 800 |
| | | AAuudTsdT | | | AAUdTsdT | |
| XD-09084 | X29126 | uuuGAcGuGAAGcaA | 663 | X29127 | cAAUUUGCUUcACGUcA | 801 |
| | | AuuGdTsdT | | | AAdTsdT | |
| XD-09085 | X29128 | uuGAcGuGAAGcAaA | 664 | X29129 | CcAAUUUGCUUcACGUc | 802 |
| | | uuGGdTsdT | | | AAdTsdT | |
| XD-09086 | X29130 | ugAcGuGAAGcAAauu | 665 | X29131 | CCcAAUUUGCUUcACGU | 803 |
| | | GGGdTsdT | | | cAdTsdT | |
| XD-09087 | X29132 | gacGuGAAGcAAAuu | 666 | X29133 | CCCcAAUUUGCUUcACG | 804 |
| | | GGGGdTsdT | | | UCdTsdT | |
| XD-09088 | X29134 | acGuGAAGcAAAuuG | 667 | X29135 | GCCCcAAUUUGCUUcAC | 805 |
| | | GGGcdTsdT | | | GUdTsdT | |
| XD-09089 | X29136 | cguGAAGcAAAuugG | 668 | X29137 | GGCCCcAAUUUGCUUcA | 806 |
| | | GGccdTsdT | | | CGdTsdT | |
| XD-09090 | X29138 | gaAGcAAAuuGGGgcc | 669 | X29139 | UUGGGCCCcAAUUUGC | 807 |
| | | cAAdTsdT | | | UUCdTsdT | |
| XD-09091 | X29140 | aaGcAAAuuGGGGccc | 670 | X29141 | GUUGGGCCCcAAUUUG | 808 |
| | | AAcdTsdT | | | CUUdTsdT | |
| XD-09092 | X29142 | agcAAAuuGGGGcccA | 671 | X29143 | AGUUGGGCCCcAAUUU | 809 |
| | | AcudTsdT | | | GCUdTsdT | |
| XD-09093 | X29144 | gcAAAuuGGGGcccA | 672 | X29145 | GAGUUGGGCCCcAAUU | 810 |
| | | AcucdTsdT | | | UGCdTsdT | |
| XD-09094 | X29146 | ugcAAAAAGcuGAaG | 673 | X29147 | AGcACUUcAGCUUUUU | 811 |
| | | uGcudTsdT | | | GcAdTsdT | |
| XD-09095 | X29148 | aaAGcuGAAGuGcuuG | 674 | X29149 | AGUcAAGcACUUcAGCU | 812 |
| | | AcudTsdT | | | UUdTsdT | |
| XD-09096 | X29150 | ugcuuGAcuGAcAuuuA | 675 | X29151 | GGuAAAUGUcAGUcAAG | 813 |
| | | ccdTsdT | | | cAdTsdT | |
| XD-09097 | X29152 | cuuGAcuGAcAuuuAcc | 676 | X29153 | cAGGuAAAUGUcAGUcA | 814 |
| | | uGdTsdT | | | AGdTsdT | |
| XD-09098 | X29154 | ugcuuuuucuuAuuAcucu | 677 | X29155 | AGAGuAAuAAGAAAAA | 815 |
| | | dTsdT | | | GcAdTsdT | |
| XD-09099 | X29156 | uucuuAuuAcucucccAu | 678 | X29157 | AAUGGGAGAGuAAuAA | 816 |
| | | udTsdT | | | GAAdTsdT | |
| XD-09100 | X29158 | cucccAuuGuGGGcucA | 679 | X29159 | AGUGAGCCcAcAAUGG | 817 |
| | | cudTsdT | | | GAGdTsdT | |
| XD-09101 | X29160 | cccAuuGuGGGcucAcu | 680 | X29161 | AGAGUGAGCCcAcAAU | 818 |
| | | cudTsdT | | | GGGdTsdT | |
| XD-09102 | X29162 | cacucuGcuGcAAauGA | 681 | X29163 | ACUcAUUUGcAGcAGAG | 819 |
| | | GudTsdT | | | UGdTsdT | |
| XD-09103 | X29164 | acucuGcuGcAAAuGA | 682 | X29165 | cACUcAUUUGcAGcAGA | 820 |
| | | GuGdTsdT | | | GUdTsdT | |
| XD-09104 | X29166 | aguGGGucuuuGGgAA | 683 | X29167 | GcAUUCCcAAAGACCcA | 821 |
| | | uGcdTsdT | | | CUdTsdT | |
| XD-09105 | X29168 | guGGGucuuuGGGaAu | 684 | X29169 | UGcAUUCCcAAAGACCc | 822 |
| | | GcAdTsdT | | | ACdTsdT | |
| XD-09106 | X29170 | ggGucuuuGGGAAuGc | 685 | X29171 | AUUGcAUUCCcAAAGA | 823 |
| | | AAudTsdT | | | CCCdTsdT | |
| XD-09107 | X29172 | uuGGGAAuGcAAugu | 686 | X29173 | UUGcAcAUUGcAUUCCc | 824 |
| | | GcAAdTsdT | | | AAdTsdT | |
| XD-09108 | X29174 | guGcAAAuuAuucacA | 687 | X29175 | CCCUGUGAAuAAUUUG | 825 |
| | | GGGdTsdT | | | cACdTsdT | |
| XD-09109 | X29176 | cacAGGGcuGuAucAc | 688 | X29177 | GAUGUGAuAcAGCCCU | 826 |
| | | AucdTsdT | | | GUGdTsdT | |
| XD-09110 | X29178 | acAGGGcuGuAucacAu | 689 | X29179 | CGAUGUGAuAcAGCCC | 827 |
| | | cGdTsdT | | | UGUdTsdT | |
| XD-09111 | X29180 | caGGGcuGuAucAcAuc | 690 | X29181 | CCGAUGUGAuAcAGCCC | 828 |
| | | GGdTsdT | | | UGdTsdT | |
| XD-09112 | X29182 | agGGcuGuAucAcaucG | 691 | X29183 | ACCGAUGUGAuAcAGC | 829 |
| | | GudTsdT | | | CCUdTsdT | |
| XD-09113 | X29184 | ggcuGuAucAcAucGGu | 692 | X29185 | uAACCGAUGUGAuAcAG | 830 |
| | | uAdTsdT | | | CCdTsdT | |
| XD-09114 | X29186 | gcuGuAucAcAucgGuu | 693 | X29187 | AuAACCGAUGUGAuAcA | 831 |
| | | AudTsdT | | | GCdTsdT | |
| XD-09115 | X29188 | cuGuAucAcAucGguuA | 694 | X29189 | AAuAACCGAUGUGAuAc | 832 |
| | | uudTsdT | | | AGdTsdT | |
| XD-09116 | X29190 | uguAucAcAucGGuuAu | 695 | X29191 | AAAuAACCGAUGUGAu | 833 |
| | | uudTsdT | | | AcAdTsdT | |
| XD-09117 | X29192 | guAucAcAucGGuuAuu | 696 | X29193 | AAAAuAACCGAUGUGA | 834 |
| | | uudTsdT | | | uACdTsdT | |
| XD-09118 | X29194 | uaucAcAucGGuuauuuu | 697 | X29195 | cAAAAuAACCGAUGUG | 835 |
| | | GdTsdT | | | AuAdTsdT | |
| XD-09119 | X29196 | ucAcAucGGuuAuuuuG | 698 | X29197 | GCcAAAAuAACCGAUG | 836 |
| | | GcdTsdT | | | UGAdTsdT | |
| XD-09120 | X29198 | acAucGGuuAuuuuGGc | 699 | X29199 | CCGCcAAAAuAACCGAU | 837 |
| | | GGdTsdT | | | GUdTsdT | |
| XD-09121 | X29200 | caucGGuuAuuuugGcG | 700 | X29201 | UCCGCcAAAAuAACCGA | 838 |
| | | GAdTsdT | | | UGdTsdT | |
| XD-09122 | X29202 | aucGGuuAuuuuGgcGG | 701 | X29203 | UUCCGCcAAAAuAACCG | 839 |
| | | AAdTsdT | | | AUdTsdT | |
| XD-09123 | X29204 | ucGGuuAuuuuGGcGG | 702 | X29205 | AUUCCGCcAAAAuAACC | 840 |
| | | AAudTsdT | | | GAdTsdT | |
| XD-09124 | X29206 | cgGuuAuuuuGGcgGA | 703 | X29207 | GAUUCCGCcAAAAuAA | 841 |
| | | AucdTsdT | | | CCGdTsdT | |
| XD-09125 | X29208 | guuAuuuuGGcGGaAuc | 704 | X29209 | AAGAUUCCGCcAAAAu | 842 |
| | | uudTsdT | | | AACdTsdT | |
| XD-09126 | X29210 | uuAuuuuGGcGGAaucu | 705 | X29211 | GAAGAUUCCGCcAAAA | 843 |
| | | ucdTsdT | | | uAAdTsdT | |
| XD-09127 | X29212 | uauuuuGGcGGAAucuu | 706 | X29213 | AGAAGAUUCCGCcAAA | 844 |
| | | cudTsdT | | | AuAdTsdT | |
| XD-09128 | X29214 | auuuuGGcGGAAucuuc | 707 | X29215 | AAGAAGAUUCCGCcAA | 845 |
| | | uudTsdT | | | AAUdTsdT | |
| XD-09129 | X29216 | uuuuGGcGGAAucuucu | 708 | X29217 | GAAGAAGAUUCCGCcA | 846 |
| | | ucdTsdT | | | AAAdTsdT | |
| XD-09130 | X29218 | uuuGGcGGAAucuucuu | 709 | X29219 | UGAAGAAGAUUCCGCc | 847 |
| | | cAdTsdT | | | AAAdTsdT | |
| XD-09131 | X29220 | aaGuGuGAucAccuGG | 710 | X29221 | cAACcAGGUGAUcAcAC | 848 |
| | | uuGdTsdT | | | UUdTsdT | |
| XD-09132 | X29222 | uguGAucAccuGGuuG | 711 | X29223 | cACcAACcAGGUGAUcAc | 849 |
| | | GuGdTsdT | | | AdTsdT | |
| XD-09133 | X29224 | guGAucAccuGGuuGG | 712 | X29225 | CcACcAACcAGGUGAUc | 850 |
| | | uGGdTsdT | | | ACdTsdT | |
| XD-09134 | X29226 | ugAucAccuGGuugGuG | 713 | X29227 | GCcACcAACcAGGUGAU | 851 |
| | | GcdTsdT | | | cAdTsdT | |
| XD-09135 | X29228 | ucAccuGGuuGGugGcu | 714 | X29229 | AcAGCcACcAACcAGGU | 852 |
| | | GudTsdT | | | GAdTsdT | |
| XD-09136 | X29230 | caccuGGuuGGuGgcuG | 715 | X29231 | cAcAGCcACcAACcAGGU | 853 |
| | | uGdTsdT | | | GdTsdT | |
| XD-09137 | X29232 | agGAAucAucuuuacuA | 716 | X29233 | UUuAGuAAAGAUGAUU | 854 |
| | | AAdTsdT | | | CCUdTsdT | |
| XD-09138 | X29234 | ucAucuuuAcuAAauGc | 717 | X29235 | UGGcAUUuAGuAAAGA | 855 |
| | | cAdTsdT | | | UGAdTsdT | |
| XD-09139 | X29236 | gaAGAuucuGuuuauGu | 718 | X29237 | AGAcAuAAAcAGAAUCU | 856 |
| | | cudTsdT | | | UCdTsdT | |
| XD-09140 | X29238 | cuGuGGcccuuAuuuucc | 719 | X29239 | UGGAAAAuAAGGGCcAc | 857 |
| | | AdTsdT | | | AGdTsdT | |
| XD-09141 | X29240 | ugGcccuuAuuuuccAcG | 720 | X29241 | UCGUGGAAAAuAAGGG | 858 |
| | | AdTsdT | | | CcAdTsdT | |
| XD-09142 | X29242 | ggcccuuAuuuuccAcGA | 721 | X29243 | CUCGUGGAAAAuAAGG | 859 |
| | | GdTsdT | | | GCCdTsdT | |
| XD-09143 | X29244 | gcccuuAuuuuccacGAG | 722 | X29245 | CCUCGUGGAAAAuAAG | 860 |
| | | GdTsdT | | | GGCdTsdT | |
| XD-09144 | X29246 | cuuAuuuuccAcGaGGA | 723 | X29247 | cAUCCUCGUGGAAAAu | 861 |
| | | uGdTsdT | | | AAGdTsdT | |
| XD-09145 | X29248 | uuAuuuuccAcGAgGAu | 724 | X29249 | CcAUCCUCGUGGAAAA | 862 |
| | | GGdTsdT | | | uAAdTsdT | |
| XD-09146 | X29250 | uauuuuccAcGAGgAuG | 725 | X29251 | UCcAUCCUCGUGGAAA | 863 |
| | | GAdTsdT | | | AuAdTsdT | |
| XD-09147 | X29252 | uuuccAcGAGGAugGA | 726 | X29253 | uAUUCcAUCCUCGUGG | 864 |
| | | AuAdTsdT | | | AAAdTsdT | |
| XD-09148 | X29254 | uuccAcGAGGAuGgA | 727 | X29255 | UuAUUCcAUCCUCGUG | 865 |
| | | AuAAdTsdT | | | GAAdTsdT | |
| XD-09149 | X29256 | uccAcGAGGAuGGaA | 728 | X29257 | AUuAUUCcAUCCUCGU | 866 |
| | | uAAudTsdT | | | GGAdTsdT | |
| XD-09150 | X29258 | cacGAGGAuGGAAuA | 729 | X29259 | AAAUuAUUCcAUCCUC | 867 |
| | | AuuudTsdT | | | GUGdTsdT | |
| XD-09151 | X29260 | aauuuccAcAcAAuAAu | 730 | X29261 | UcAUuAUUGUGUGGAA | 868 |
| | | GAdTsdT | | | AUUdTsdT | |
| XD-09152 | X29262 | uuuccAcAcAAuAauG | 731 | X29263 | CCUcAUuAUUGUGUGG | 869 |
| | | AGGdTsdT | | | AAAdTsdT | |
| XD-09153 | X29264 | cacAcAAuAAuGAgG | 732 | X29265 | UGUUCCUcAUuAUUGU | 870 |
| | | AAcAdTsdT | | | GUGdTsdT | |
| XD-09154 | X29266 | uaAuGAGGAAcAuuuu | 733 | X29267 | CCcAAAAUGUUCCUcAU | 871 |
| | | GGGdTsdT | | | uAdTsdT | |
| XD-09155 | X29268 | aacAuuuuGGGGcuGG | 734 | X29269 | GGACcAGCCCcAAAAUG | 872 |
| | | uccdTsdT | | | UUdTsdT | |
| XD-09156 | X29270 | gccGcuGcucAucauGGu | 735 | X29271 | GACcAUGAUGAGcAGC | 873 |
| | | cdTsdT | | | GGCdTsdT | |
| XD-09157 | X29272 | aauucuucGGccugAGu | 736 | X29273 | UuACUcAGGCCGAAGA | 874 |
| | | AAdTsdT | | | AUUdTsdT | |
| XD-09158 | X29274 | auucuucGGccuGaGuA | 737 | X29275 | GUuACUcAGGCCGAAG | 875 |
| | | AcdTsdT | | | AAUdTsdT | |
| XD-09159 | X29276 | uucuucGGccuGAguAA | 738 | X29277 | AGUuACUcAGGCCGAA | 876 |
| | | cudTsdT | | | GAAdTsdT | |
| XD-09160 | X29278 | uucGGccuGAGuAacu | 739 | X29279 | cAcAGUuACUcAGGCCG | 877 |
| | | GuGdTsdT | | | AAdTsdT | |
| XD-09161 | X29280 | ugAGuAAcuGuGAaA | 740 | X29281 | GUGCUUUcAcAGUuACU | 878 |
| | | GcAcdTsdT | | | cAdTsdT | |
| XD-09162 | X29282 | caccAGucAAcuGgAcc | 741 | X29283 | UUGGUCcAGUUGACUG | 879 |
| | | AAdTsdT | | | GUGdTsdT | |
| XD-09163 | X29284 | caAcuGGAccAAGccA | 742 | X29285 | GCGUGGCUUGGUCcAG | 880 |
| | | cGcdTsdT | | | UUGdTsdT | |
| XD-09164 | X29286 | aacuGGAccAAGccAc | 743 | X29287 | UGCGUGGCUUGGUCcA | 881 |
| | | GcAdTsdT | | | GUUdTsdT | |

### Other Antagonists of CCR2 and CSF1R

In some embodiments, the antagonists of CCR2 and CSF1R can be inhibitory oligonucleotides, antibody antagonists of CCR2 and CSF1R, small molecules, peptide antagonists, and combinations thereof for CCR2, CSF1R, or both CCR, and CSF1R.

As used herein, the term “antibody” refers to an immunoglobulin molecule with a specific amino acid sequence evoked by an antigen, e.g. CCR2 or CSF1R, and characterized by reacting specifically with the antigen. The term “antibody” encompasses polyclonal and monoclonal antibodies, CDR-grafted antibodies, hybrid antibodies, VHH antibodies, altered antibodies, F(ab)2 fragments, F(ab) molecules, Fab′ fragments, Fv fragments, single domain antibodies, ScFvs, chimeric antibodies, humanized antibodies, nanobodies, diabodies, tandem antibodies and functional fragments thereof which exhibit immunological binding properties of the parent antibody molecule.

In some embodiments, CCR2 antagonist antibodies can be the humanized CCR2 antibodies of U.S. Pat. Nos. 6,696,550 and 6,084,075; human antibodies in U.S. Pat. Nos. 9,315,579 and 9,238,691; antibodies in U.S. Pat. Publ. No. 2009/0297502; anti-CCR2 antibodies in PCT Publ. Nos. WO 2016/08180 and WO 2010/021697. The antibodies or functional fragments thereof which bind to CCR2 can also include, for example, an anti-CCR2 antibody and its fragments as described in U.S. Publ. Nos. 2002/0042370; 2004/0132980; 2004/0126851; 2005/0048052; 2008/0241923; 2009/0191192, 2011/0274696; 2013/0344070; and 2015/0086546; PCT Publ. Nos. WO 2005/060368 and WO2007/147026.

In one embodiment, the antagonist of CCR2 is an antagonist peptide such as a blocking peptide that blocks the binding of its ligand and inhibits activation of the receptor, for example, a CCR non-competitive antagonist peptide that consists of LGTFLKC (SEQ ID NO: 882) disclosed in U.S. Pat. No. 9,434,766.

In other embodiments, CCR2 antagonists can be a modified chemokine ligand, for example, a modified MCP-1 chemokine and a modified MCP-5 chemokine.

The antagonists of CCR2 can also include a range of small molecule antagonists of CCR2, including, but not limited to compounds, for example, described in U.S. Pat. Nos. 8,546,408; 8,575,173 and 9,394,307; U.S. Pat. Publ. Nos. 2010/0056509 and 2011/0118248; PCT Publ. Nos. WO2004/069809, WO2005/118578, WO2006/012135, WO2007/130712, WO2007/014008, WO 2008/008374, WO2008/109238, WO2008/008375, WO2010/008761, WO2011/156554, WO2011/159854, WO2011/042399, WO2012/125661, WO2012/125662, WO2012/125663, WO2013/111129, WO2013/152269, WO2014/014901, and WO2016/187393.

In some embodiments, the antagonists of CSF1R can be antibodies and their functional fragments and variants; other inhibitory nucleic acid molecules such as oligonucleotides and aptamers; small molecules; and competitive ligands such as CSF1R extracellular domain (ECD) fusion molecules.

In some embodiments, CSF1R antagonist antibodies can include, but are not limited to, anti-CSF1R antibodies in U.S. Pat. Nos. 8,747,845 and 9,200,075; antibodies that bind CSF1R in PCT Publ. Nos. WO 2011/140294, WO 2016/168149, and WO 2016/106180; anti-CSF1R antibodies in U.S. Pat. Publ. Nos 2017/0081415 and 2017/0152320.

CSF1R inhibitors can include, but are not limited to, CSF1R inhibitors, such as GW2580, KI20227, HY-13075, cFMS Receptor Inhibitor II, cFMS Receptor Inhibitor III, cFMS Receptor Inhibitor IV or ARRY-382 (e.g., U.S. Pat. Publ. No. 2016/0032248). The CSF1R inhibitors can also comprise the compounds discussed in U.S. Pat. Nos. 8,648,086 and 9,452,167; inhibitors screened in PCT Publ. No. WO 2009/075344.

In some embodiments, CSF1R antagonist can be a CSF1R ECD-Fc fusion protein as described in U.S. Pat. No. 8,080,246.

In some embodiments, the siRNA molecules encompassed by the present invention can be combined with other antagonists of CCR2 and CSF1R. In one embodiment, the siRNA molecules specific to CCR2 can be combined with another antagonist of CSF1R to form combined antagonists. In another embodiment, the siRNA molecules specific to CCR2 can be combined with another antagonist of CCR2 (e.g., an anti-CCR2 antibody) to achieve a dual inhibition of CCR2.

### Cell-Based Compositions

In some embodiments, cell-based agents are used. For example, myeloid-derived cells contacted with agents described herein can be administered.

Cell-based agents have an immunocompatibility relationship to a subject host and any such relationship is contemplated for use according to the present invention. For example, the cells, such as adoptive monocytes and/or macrophages, T cells, and the like, can be syngeneic. The term “syngeneic” can refer to the state of deriving from, originating in, or being members of the same species that are genetically identical, particularly with respect to antigens or immunological reactions. These include identical twins having matching MHC types. Thus, a “syngeneic transplant” refers to transfer of cells from a donor to a recipient who is genetically identical to the donor or is sufficiently immunologically compatible as to allow for transplantation without an undesired adverse immunogenic response (e.g., such as one that would work against interpretation of immunological screen results described herein).

A syngeneic transplant can be “autologous” if the transferred cells are obtained from and transplanted to the same subject. An “autologous transplant” refers to the harvesting and reinfusion or transplant of a subject's own cells or organs. Exclusive or supplemental use of autologous cells can eliminate or reduce many adverse effects of administration of the cells back to the host, particular graft versus host reaction.

A syngeneic transplant can be “matched allogeneic” if the transferred cells are obtained from and transplanted to different members of the same species yet have sufficiently matched major histocompatibility complex (MEW) antigens to avoid an adverse immunogenic response. Determining the degree of MEW mismatch can be accomplished according to standard tests known and used in the art. For instance, there are at least six major categories of MEW genes in humans, identified as being important in transplant biology. HLA-A, HLA-B, HLA-C encode the HLA class I proteins while HLA-DR, HLA-DQ, and HLA-DP encode the HLA class II proteins. Genes within each of these groups are highly polymorphic, as reflected in the numerous HLA alleles or variants found in the human population, and differences in these groups between individuals is associated with the strength of the immune response against transplanted cells. Standard methods for determining the degree of MHC match examine alleles within HLA-B and HLA-DR, or HLA-A, HLA-B and HLA-DR groups. Thus, tests can be made of at least 4, and even 5 or 6 MEW antigens within the two or three HLA groups, respectively. In serological MEW tests, antibodies directed against each HLA antigen type are reacted with cells from one subject (e.g., donor) to determine the presence or absence of certain MHC antigens that react with the antibodies. This is compared to the reactivity profile of the other subject (e.g., recipient). Reaction of the antibody with an MEW antigen is typically determined by incubating the antibody with cells, and then adding complement to induce cell lysis (i.e., lymphocytotoxicity testing). The reaction is examined and graded according to the amount of cells lysed in the reaction (see, for example, Mickelson and Petersdorf (1999) Hematopoietic Cell Transplantation, Thomas, E. D. et al. eds., pg 28-37, Blackwell Scientific, Malden, Mass.). Other cell-based assays include flow cytometry using labeled antibodies or enzyme linked immunoassays (ELISA). Molecular methods for determining MEW type are well-known and generally employ synthetic probes and/or primers to detect specific gene sequences that encode the HLA protein. Synthetic oligonucleotides can be used as hybridization probes to detect restriction fragment length polymorphisms associated with particular HLA types (Vaughn (2002) Method. Mol. Biol. MHC Protocol. 210:45-60). Alternatively, primers can be used for amplifying the HLA sequences (e.g., by polymerase chain reaction or ligation chain reaction), the products of which can be further examined by direct DNA sequencing, restriction fragment polymorphism analysis (RFLP), or hybridization with a series of sequence specific oligonucleotide primers (SSOP) (Petersdorf et al. (1998) Blood 92:3515-3520; Morishima et al. (2002) Blood 99:4200-4206; and Middleton and Williams (2002) Method. Mol. Biol. MHC Protocol. 210:67-112).

A syngeneic transplant can be “congenic” if the transferred cells and cells of the subject differ in defined loci, such as a single locus, typically by inbreeding. The term “congenic” refers to deriving from, originating in, or being members of the same species, where the members are genetically identical except for a small genetic region, typically a single genetic locus (i.e., a single gene). A “congenic transplant” refers to transfer of cells or organs from a donor to a recipient, where the recipient is genetically identical to the donor except for a single genetic locus. For example, CD45 exists in several allelic forms and congenic mouse lines exist in which the mouse lines differ with respect to whether the CD45.1 or CD45.2 allelic versions are expressed.

By contrast, “mismatched allogeneic” refers to deriving from, originating in, or being members of the same species having non-identical major histocompatibility complex (MEW) antigens (i.e., proteins) as typically determined by standard assays used in the art, such as serological or molecular analysis of a defined number of MEW antigens, sufficient to elicit adverse immunogenic responses. A “partial mismatch” refers to partial match of the MEW antigens tested between members, typically between a donor and recipient. For instance, a “half mismatch” refers to 50% of the MEW antigens tested as showing different MHC antigen type between two members. A “full” or “complete” mismatch refers to all MEW antigens tested as being different between two members.

Similarly, in contrast, “xenogeneic” refers to deriving from, originating in, or being members of different species, e.g., human and rodent, human and swine, human and chimpanzee, etc. A “xenogeneic transplant” refers to transfer of cells or organs from a donor to a recipient where the recipient is a species different from that of the donor.

In addition, cells can be obtained from a single source or a plurality of sources (e.g., a single subject or a plurality of subjects). A plurality refers to at least two (e.g., more than one). In still another embodiment, the non-human mammal is a mouse. The animals from which cell types of interest are obtained can be adult, newborn (e.g., less than 48 hours old), immature, or in utero. Cell types of interest can be primary cancer cells, cancer stem cells, established cancer cell lines, immortalized primary cancer cells, and the like. In certain embodiments, the immune systems of host subjects can be engineered or otherwise elected to be immunological compatible with transplanted cancer cells. For example, in one embodiment, the subject can be “humanized” in order to be compatible with human cancer cells. The term “immune-system humanized” refers to an animal, such as a mouse, comprising human HSC lineage cells and human acquired and innate immune cells, survive without being rejected from the host animal, thereby allowing human hematopoiesis and both acquired and innate immunity to be reconstituted in the host animal. Acquired immune cells include T cells and B cells. Innate immune cells include macrophages, granulocytes (basophils, eosinophils, neutrophils), DCs, NK cells and mast cells. Representative, non-limiting examples include SCID-hu, Hu-PBL-SCID, Hu-SRC-SCID, NSG (NOD-SCID IL2r-gamma(null) lack an innate immune system, B cells, T cells, and cytokine signaling), NOG (NOD-SCID IL2r-gamma(truncated)), BRG (BALB/c-Rag2(null)IL2r-gamma(null)), and H2dRG (Stock-H2d-Rag2(null)IL2r-gamma(null)) mice (see, for example, Shultz et al. (2007) Nat. Rev. Immunol. 7:118; Pearson et al. (2008) Curr. Protocol. Immunol. 15:21; Brehm et al. (2010) Clin. Immunol. 135:84-98; McCune et al. (1988) Science 241:1632-1639, U.S. Pat. No. 7,960,175, and U.S. Pat. Publ. No. 2006/0161996), as well as related null mutants of immune-related genes like Rag1 (lack B and T cells), Rag2 (lack B and T cells), TCR alpha (lack T cells), perforin (cD8+ T cells lack cytotoxic function), FoxP3 (lack functional CD4+T regulatory cells), IL2rg, or Prf1, as well as mutants or knockouts of PD-1, PD-L1, Tim3, and/or 2B4, allow for efficient engraftment of human immune cells in and/or provide compartment-specific models of immunocompromised animals like mice (see, for example, PCT Publ. No. WO 2013/062134). In addition, NSG-CD34+(NOD-SCID IL2r-gamma(null) CD34+) humanized mice are useful for studying human gene and tumor activity in animal models like mice.

As used herein, “obtained” from a biological material source means any conventional method of harvesting or partitioning a source of biological material from a donor. For example, biological material can obtained from a solid tumor, a blood sample, such as a peripheral or cord blood sample, or harvested from another body fluid, such as bone marrow or amniotic fluid. Methods for obtaining such samples are well-known to the artisan. In the present invention, the samples can be fresh (i.e., obtained from a donor without freezing). Moreover, the samples can be further manipulated to remove extraneous or unwanted components prior to expansion. The samples can also be obtained from a preserved stock. For example, in the case of cell lines or fluids, such as peripheral or cord blood, the samples can be withdrawn from a cryogenically or otherwise preserved bank of such cell lines or fluid. Such samples can be obtained from any suitable donor.

The obtained populations of cells can be used directly or frozen for use at a later date. A variety of mediums and protocols for cryopreservation are known in the art. Generally, the freezing medium will comprise DMSO from about 5-10%, 10-90% serum albumin, and 50-90% culture medium. Other additives useful for preserving cells include, by way of example and not limitation, disaccharides such as trehalose (Scheinkonig et al. (2004) Bone Marrow Transplant. 34:531-536), or a plasma volume expander, such as hetastarch (i.e., hydroxyethyl starch). In some embodiments, isotonic buffer solutions, such as phosphate-buffered saline, can be used. An exemplary cryopreservative composition has cell-culture medium with 4% HSA, 7.5% dimethyl sulfoxide (DMSO), and 2% hetastarch. Other compositions and methods for cryopreservation are well-known and described in the art (see, e.g., Broxmeyer et al. (2003) Proc. Natl. Acad. Sci. U.S.A. 100:645-650). Cells are preserved at a final temperature of less than about −135° C.

In some embodiments, useful agents can be CAR (chimeric antigen receptor)-T therapy, where T cells engineered to express CARs comprising an antigen-binding domain specific to an antigen on tumor cells of interest. The term “chimeric antigen receptor” or “CAR” refers to receptors having a desired antigen specificity and signaling domains to propagate intracellular signals upon antigen binding. For example, T lymphocytes recognize specific antigens through interaction of the T cell receptor (TCR) with short peptides presented by major histocompatibility complex (MHC) class I or II molecules. For initial activation and clonal expansion, naive T cells are dependent on professional antigen-presenting cells (APCs) that provide additional co-stimulatory signals. TCR activation in the absence of co-stimulation can result in unresponsiveness and clonal anergy. To bypass immunization, different approaches for the derivation of cytotoxic effector cells with grafted recognition specificity have been developed. CARs have been constructed that consist of binding domains derived from natural ligands or antibodies specific for cell-surface components of the TCR-associated CD3 complex. Upon antigen binding, such chimeric antigen receptors link to endogenous signaling pathways in the effector cell and generate activating signals similar to those initiated by the TCR complex. Since the first reports on chimeric antigen receptors, this concept has steadily been refined and the molecular design of chimeric receptors has been optimized and routinely use any number of well-known binding domains, such as scFV, Fav, and another protein binding fragments described herein.

In some embodiments, monocytes and macrophages can be engineered to, for example, express a chimeric antigen receptor (CAR). The modified cell can be recruited to the tumor microenvironment where it acts as a potent immune effector by infiltrating the tumor and killing target cancer cells. The CAR includes an antigen binding domain, a transmembrane domain and an intracellular domain. The antigen binding domain binds to an antigen on a target cell. Examples of cell surface markers that can act as an antigen that binds to the antigen binding domain of the CAR include those associated with viral, bacterial, parasitic infections, autoimmune disease and cancer cells (e.g., tumor antigens).

In one embodiment, the antigen binding domain binds to a tumor antigen, such as an antigen that is specific for a tumor or cancer of interest. Non-limiting examples of tumor associated antigens include BCMA, CD19, CD24, CD33, CD38; CD44v6, CD123, CD22, CD30, CD117, CD171, CEA, CS-1, CLL-1, EGFR, ERBB2, EGFRvIII, FLT3, GD2, NY-BR-1, NY-ESO-1, p53, PRSS21, PSMA, ROR1, TAG72, Tn Ag, VEGFR2.

In one embodiment, the transmembrane domain is naturally associated with one or more of the domains in the CAR. The transmembrane domain can be derived either from a natural or from a synthetic source. Transmembrane regions of particular use in this invention can be derived from (i.e. comprise at least the transmembrane region(s) of) the alpha, beta or zeta chain of the T-cell receptor, CD28, CD3 epsilon, CD45, CD4, CD5, CD8, CD9, CD 16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and TLR9. In some instances, a variety of human hinges can be employed as well including the human Ig (immunoglobulin) hinge.

In one embodiment, the intracellular domain of the CAR includes a domain responsible for signal activation and/or transduction. Examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP 12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD 1 id, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

In some embodiments, agents, compositions and methods encompassed by the present invention can be used to re-engineer monocytes and macrophages to increase their ability to present antigens to other immune effector cells, for example, T cells. Engineered monocytes and macrophages as antigen presenting cells (APCs) will process tumor antigens and present antigenic epitopes to T cells to stimulate adaptive immune responses to attack tumor cells.

### IV. Formulations

In some embodiments, the oligonucleotide compositions encompassed by the present invention comprising siRNA molecules specific to CCR2 are used alone as therapeutic agents. In other embodiments, the oligonucleotide compositions encompassed by the present invention comprising siRNA molecules specific to CSF1R can be used alone as therapeutic agents.

In still other embodiments, the oligonucleotide compositions encompassed by the present invention comprising siRNA molecules specific to CCR2 and CSF1R are used in combination. In some examples, the siRNA molecules specific to CCR2 and CSF1R can form a siRNA molecule cocktail. The siRNA molecules specific to CCR2 and the siRNA molecules specific to CSF1R can be present in the siRNA molecule cocktail composition at a ratio from 1:1 to 1:10. Alternatively, the siRNA molecules specific to CSF1R and the siRNA molecules specific to CCR2 can be present in the siRNA molecule cocktail composition at a ratio from 1:1 to 1:10. In other embodiments, the siRNA molecules specific to CCR2 and CSF1R can be incorporated with a complex of macromolecular assemblies or pharmaceutical compositions. The siRNA molecules encompassed by the present invention can be formulated as a variety of pharmaceutical compositions.

The pharmaceutical compositions will be prepared in a form appropriate for the desired use, such as in vitro, ex vivo, or in vivo administration and include an effective amount of pharmacologically active compound encompassed by the present invention, alone or in combination with one or more pharmaceutically acceptable carriers.

The siRNA molecule cocktail composition comprising the siRNA molecules specific to CCR2 and the siRNA molecules specific to CSF1R encompassed by the present invention can be used to suppress the expression of CCR2 and CSF1R receptors, and/or to inhibit the activity of CCR2 and CSF1R.

In some embodiments, the siRNA composition encompassed by the present invention can further comprise an antagonist against the ligands of CCR2 and CSF1R, such as CCL2 and CSF1, respectively. In one example, the composition encompassed by the present invention can comprise siRNA molecules specific to CCR2 in combination with a CCL2 antagonist; the CCL2 antagonist can be a siRNA molecule specific to CCL2, an anti-CCL2 antibody and/or a small molecule. In another example, the composition encompassed by the present invention can comprise siRNA molecules specific to CSF1R in combination with a CSF1 antagonist; the CSF1 antagonist can be a siRNA molecule specific to CSF1, an anti-CSF1 antibody and/or a small molecule.

In some embodiments, the siRNA cocktail composition comprising the siRNA molecules specific to CCR2 and the siRNA molecules specific to CSF1R encompassed by the present invention can further comprise one or more agents, such as those that target monocytes and macrophages, those that stimulate immune responses, and the like. Such monocyte/macrophage targeting drugs can include, but are not limited to, rovelizumab which targets CD11b, small molecules MNRP1685A that targets Neurophilin-1, nesvcumab targeting ANG2, pascolizumab specific to IL-4, dupilumab specific to IL4Ra, tocilizumab and sarilumab specific to IL-6R, adalimumab, certolizumab, tanercept, golimumab, and infliximab specific to TNF-α, and CP-870 and CP-893 targeting CD40.

In some embodiments, the oligonucleotide compositions comprising siRNA molecules specific to CCR2 and/or siRNA molecules specific to CSF1R encompassed by the present invention can be used as naked compositions. In other embodiments, the oligonucleotide compositions encompassed by the present invention can be formulated as combined agents.

In some embodiments, the pharmaceutical compositions comprising the oligonucleotide compositions encompassed by the present invention can be formulated with one or more agents that can enhance the uptake of oligonucleotides at the cellular level, such as for the transport of oligomers across a cell membrane.

A composition in accordance with the invention can be prepared, packaged, and/or sold in bulk, as a single unit dose, and/or as a plurality of single unit doses. As used herein, a “unit dose” is discrete amount of the pharmaceutical composition comprising a pre-determined amount of the active ingredient. The amount of the active ingredient is generally equal to the dosage of the active ingredient which would be administered to a subject and/or a convenient fraction of such a dosage such as, for example, one-half or one-third of such a dosage.

### Lipid-Based Formulations

In some embodiments, lipid-based formulations are used. Accordingly, provided herein are lipid-based formulations comprising a composition as described herein and one or more lipids. In some embodiments, the lipid is a lipid particle or amphiphilic compound. The lipid can be neutral, anionic, or cationic at physiologic pH.

Suitable solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40, preferably 12-30 carbon atoms, such as glyceryl monostearate, glycerol behenate, glycerol palmitostearate, glycerol trilaurate, tricaprin, trilaurin, trimyristin, tripalmitin, tristearin, and hydrogenated castor oil. Suitable solid lipids can include cetyl palmitate, beeswax, or cyclodextrin.

Amphiphilic compounds include, but are not limited to, phospholipids, such as 1,2 distearoyl-sn-glycero-3-phosphoethanolamine (DSPE), dipalmitoylphosphatidylcholine (DPPC), di stearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), for example, between 0.1-30 (weight lipid/w polymer). Phospholipids which can be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-γ-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), di stearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) can also be used.

In some embodiments, lipid-based particles are used. The term “lipid particles” refers to liposomes, lipid micelles, solid lipid particles, lipoplexes, lipid nanoparticles (LNPs), or lipid-stabilized polymeric particles, composed of one or a mixture of different biocompatible lipids, e.g., at least one or more cationic lipids and/or one or more neutral lipids and/or polyethylene glycol (PEG)-lipids.

The particle can be a lipid micelle. Lipid micelles can be formed, for instance, as a water-in-oil emulsion with a lipid surfactant. An emulsion is a blend of two immiscible phases wherein a surfactant is added to stabilize the dispersed droplets. In some embodiments the lipid micelle is a microemulsion. A microemulsion is a thermodynamically stable system composed of at least water, oil and a lipid surfactant producing a transparent and thermodynamically stable system whose droplet size is less than 1 micron, from about 10 nm to about 500 nm, or from about 10 nm to about 250 nm. Lipid micelles are generally useful for encapsulating hydrophobic active agents, including hydrophobic therapeutic agents, hydrophobic prophylactic agents, or hydrophobic diagnostic agents.

The particle can be a solid lipid particle. Solid lipid particles present an alternative to the colloidal micelles and liposomes. Solid lipid particles are typically submicron in size, i.e. from about 10 nm to about 1 micron, from 10 nm to about 500 nm, or from 10 nm to about 250 nm. Solid lipid particles are formed of lipids that are solids at room temperature. They are derived from oil-in-water emulsions, by replacing the liquid oil by a solid lipid.

The particle can be a liposome. Liposomes are small vesicles composed of an aqueous medium surrounded by lipids arranged in spherical bilayers. Liposomes can be classified as small unilamellar vesicles, large unilamellar vesicles, or multi-lamellar vesicles. Multi-lamellar liposomes contain multiple concentric lipid bilayers. Liposomes can be used to encapsulate agents, by trapping hydrophilic agents in the aqueous interior or between bilayers, or by trapping hydrophobic agents within the bilayer.

The lipid micelles and liposomes typically have an aqueous center. The aqueous center can contain water or a mixture of water and alcohol. Suitable alcohols include, but are not limited to, methanol, ethanol, propanol, (such as isopropanol), butanol (such as n-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

Liposomes are artificially-prepared vesicles which can primarily be composed of a lipid bilayer and can be used as a delivery vehicle for the administration of nutrients and pharmaceutical formulations. Liposomes can be of different sizes such as, but not limited to, a multilamellar vesicle (MLV) which can be hundreds of nanometers in diameter and can contain a series of concentric bilayers separated by narrow aqueous compartments, a small unicellular vesicle (SUV) which can be smaller than 50 nm in diameter, and a large unilamellar vesicle (LUV) which can be between 50 and 500 nm in diameter. Liposome design can include, but is not limited to, opsonins or ligands in order to improve the attachment of liposomes to unhealthy tissue or to activate events such as, but not limited to, endocytosis. Liposomes can contain a low or a high pH in order to improve the delivery of the pharmaceutical formulations.

The formation of liposomes can depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In one embodiment, pharmaceutical compositions described herein can include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)[1,3]-dioxolane (DLin-KC2-DMA), and MC3 (e.g., as described in U.S. Pat. Publ. No. 2010/0324120).

In one embodiment, the compositions encompassed by the present invention can be formulated in a lipid-polycation complex. The formation of the lipid-polycation complex can be accomplished by methods known in the art and/or as described in U.S. Pat. Publ. No. 2012/0178702. As a non-limiting example, the polycation can include a cationic peptide or a polypeptide such as, but not limited to, polylysine, polyornithine and/or polyarginine and the cationic peptides described in PCT Publ. No. WO 2012/013326. In another embodiment, the compositions encompassed by the present invention can be formulated in a lipid-polycation complex which can further include a neutral lipid such as, but not limited to, cholesterol or dioleoyl phosphatidylethanolamine (DOPE). The liposome formulation can be influenced by, but not limited to, the selection of the cationic lipid component, the degree of cationic lipid saturation, the nature of the PEGylation, ratio of all components and biophysical parameters such as size.

In some embodiments, the lipid particle is a lipid nanoparticle (LNP). The term “lipid nanoparticle (LNP)” refers to lipid-based particles in the submicron range which include one or more lipid components as described herein. LNPs can have structural characteristics of liposomes and/or have alternative non-bilayer types of structures, which can be used to systemically deliver nucleic acid based drugs, including, for example, siRNA molecules complementary to the nucleic acid sequence of mRNA transcribed from at least one biomarker (e.g., at least one target listed in Table 1 and/or Table 2) described herein. In some embodiments, the LNP formulation comprises one or more cationic lipids. Cationic lipids are lipids that carry a net positive charge at any physiological pH. In certain particular embodiments, the LNP comprises a lipidoid as described herein. The positive charge is useful for association with negatively charged therapeutic agents, such as siRNA molecules.

In certain embodiments, a lipid nanoparticle comprises one or more lipids and a composition as described herein. In certain particular embodiments, a composition as described herein is encapsulated within a lipid nanoparticle.

In some embodiments, the sizes and charge ratios and other physical properties (e.g., membrane fluidity) of LNPs are optimized for increased cell transfection and delivery.

Lipid or lipidoid particles can comprise, for example, cationic lipids, neutral lipids, amino acid- or peptide-based lipids, polyethylene glycol (PEG)-lipids, e.g., lipids with PEG chains such as hydrogenated soybean phosphatidylcholine (HSPC), cholesterol (CHE), 1, 2-distearoyl-glycero-3-phosphoethanolamine-N-[methoxy (PEG)-2000] (DSPE-PEG2000), 1, 2-distearoyl-sn-glycero-3-phosphoethanolamine-N-[methoxy (PEG)-2000] modified with a maleimidic group in the distal end of the chain 1, 2-distearoyl-sn-glycero-3-phosphoethanolamine-N-[maleimide (PEG)-2000], DSPE-PEG2000-MAL, 1,2-dimyristoyl-sn-glycero-3-phosphoethanolamine-N-[methoxy(polyethylene glycol)-550] (DMPE-PEG550), 1, 2-dioleoyl-1-3-trimethylammonium propane (DOTAP), and those with a glycerol backbone e.g., DMG-PEG, DSG-PEG (DMG-PEG2000) etc. As used herein, a liposome is a structure comprising lipid-containing membranes enclosing an aqueous interior. For example, lipid-based formulations can be used to deliver nucleic acid agents of the present invention, e.g., siRNAs, miRNAs, oligonucleotides, modified mRNAs and other types of nucleic acid molecules.

Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-di stearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

A number of cationic lipids, and methods for making them, are described in, for example, U.S. Pat. Nos. 5,830,430; 6,056,938; 7,893,302; 7,404,969; 8,034,376; 8,283,333; and 8,642,076, as well as PCT Publ. Numbers WO 2010/054406, WO 2010/054401, WO 2010/054405, WO 2010/054384, WO 2012/040184, WO 2011/153120, WO 2011/149733, WO 2011/090965, WO 2011/043913, WO 2011/022460, WO 2012/061259, WO 2012/054365, WO 2012/044638, WO 2010/080724, WO 2010/21865, and WO 2008/103276.

The term “cationic lipid” is meant to include those lipids having one or two fatty acid or fatty aliphatic chains and an amino head group (including an alkylamino or dialkylamino group) that can be protonated to form a cationic lipid at physiological pH, which consist of a positively charged headgroup and a hydrophobic tail. The positively charged headgroup can serve to electrostatically bind the negatively charged siRNA molecule, while the hydrophobic tail leads to self-assembly into lipophilic particles. Examples of cationic lipids can include, but are not limited to: DLin-K-DMA, DLinDMA, DLinDAP, DLin-K-C2-DMA, DLin-K2-DMA, DOTAP, DMME, DOME, DOTMA, DDAB, Ethyl PC, multivalent cationic lipid and DC-cholesterol, DODA, DODMA, DSDMA, DOTMA, DDAB, DODAP, DOTAP, DOTAP-Cl, DC-Chol, DMRIE, DOSPA, DOGS, DOPE, CLinDMA, CpLinDMA, DMOBA, DOcarbDAP, DLincarbDAP, DLinCDAP. A number of these lipids and related analogs have been described in U.S. Pat. Publ. Numbers 2006/0083780 and 2006/0240554; and U.S. Pat. Nos. 5,208,036; 5,264,618; 5,279,833; 5,283,185; 5,753,613 and 5,785,992. Cationic lipids can also be a lipofectin (see, e.g., U.S. Pat. No. 5,705,188), such as Lipofectamine®, Lipofectamine 2000®, Lipofectamine 3000®, RNAiMAX®, and the like.

Other cationic lipids, which carry a net positive charge at about physiological pH, can be used in the lipid particles of the present invention, including, but not limited to, N,N-dioleyl-N,N-dimethylammonium chloride (DODAC), dioctadecyldimethylammonium (DODMA), di stearyldimethylammonium (DSDMA), N-(1-(2,3-dioleyloxy)propyl)-N,N,N-trimethylammonium chloride (DOTMA), N,N-distearyl-N,N-dimethylammonium bromide (DDAB), 1,2-dioleoyl-3-dimethylammonium propane (DODAP), N-(1-(2,3-dioleoyloxy)propyl)-N,N,N-trimethylammonium chloride (DOTAP), 1,2-Dioleyloxy-3-trimethylaminopropane chloride salt (DOTAP.Cl), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 1,2-dileoyl-sn-3-phosphoethanolamine (DOPE, which carries a positive charge at physiological pH but at acidic pH), 3-dimethylamino-2-(cholest-5-en-3-beta-oxybutan-4-oxy)-1-(cis,cis-9,12-octadecadienoxy)propane (CLinDMA), 2-[5′-(cholest-5-en-3(3-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-Dilinoleylcarbamyl-3-dimethylaminopropane (DLincarbDAP), 1,2-Dilinoleoylcarbamyl-3-dimethylaminopropane (DLinCDAP), and mixtures thereof. A number of these lipids and related analogs have been described in U.S. Pat. Publ. Nos. 2006/0083780 and 2006/0240554; U.S. Pat. Nos. 5,208,036; 5,264,618; 5,279,833; 5,283,185; 5,753,613; and 5,785,992.

Suitable additional cationic lipids can also include, but are not limited to, N-[1-(2,3-dioleoyloxy)propyl]-N,N,N-trimethyl ammonium salts, also referenced as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3 [N—(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N,N,N′,N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DOME), 1,2-dioleyloxypropyl-3-dimetyl-hydroxypropyl ammonium bromide (DOME-HP), 1,2-dioleyl-oxy-propyl-3-dimethyl-hydroxybutyl ammonium bromide (DOME-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

Cationic lipids can also be ionizable cationic lipids. Suitable ionizable cationic lipids for use in formulating a composition described herein include lipids described in WO2015/074805. Other suitable ionizable cationic lipids suitable for formulating a composition of the present invention can include those described in US 2015/0239834.

In some embodiments, symmetric or asymmetric or ionizable cationic lipids can be used in a nanoparticle or lipid formulation. Such lipids are disclosed in, for example, U.S. Pat. Publ. Nos. 2015/0239926, 2015/0239834, and 2015/0141678, and PCT Publ. No. WO 2015/074805.

Additionally, a number of commercial preparations of cationic lipids can be used, such as LIPOFECTIN® (including DOTMA and DOPE, available from GIBCO/BRL), LIPOFECTAMINE® (comprising DOSPA and DOPE, available from GIBCO/BRL), TRANSFECTIN® (from Bio-Rad Laboratories, Inc.) and siPORT NEOFX® (from Applied Biosystems).

Cationic lipids can also be modified cationic lipids suitable for cellular delivery of compositions comprising agents described herein, such as siRNA molecules (see, for example, those described in U.S. Pat. Publ. No. 2013/0323269); cationic glycerol derivatives, and polycationic molecules, such as polylysine (PCT Publ. No. WO 97/30731), cationic group including one or more biodegradable groups (U.S. Pat. Publ. No. 2013/0195920).

In some embodiments, the ionizable lipid can be ionizable amino lipids described in WO 2015/074805 or US 2015/0239834.

In certain embodiments, a composition described herein further comprises an aminoalcohol lipidoid as described in WO 2010/053572. In certain embodiments, the lipidoid compound is selected from Formulae (I)-(V):

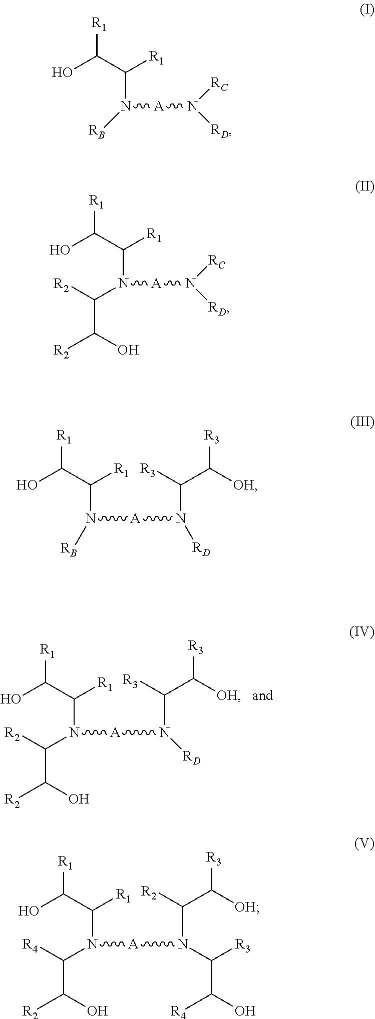

and pharmaceutically acceptable salts thereof, wherein:
A is a substituted or unsubstituted, branched or unbranched, cyclic or acyclic C₂₋₂₀ alkylene, optionally interrupted by 1 or more heteroatoms independently selected from O, S and N, or A is a substituted or unsubstituted, saturated or unsaturated 4-6-membered ring;

R₁ is hydrogen, a substituted, unsubstituted, branched or unbranched C₁₋₂₀-aliphatic or a substituted, unsubstituted, branched or unbranched C₁₋₂₀ heteroaliphatic, wherein at least one occurrence of R₁ is hydrogen;

R_{B}, R_{C}, and R_{D} are, independently, hydrogen, a substituted, unsubstituted, branched or unbranched C₁₋₂₀-aliphatic, or a substituted, unsubstituted, branched or unbranched C₁₋₂₀-heteroaliphatic or —CH₂CH(OH)R_{E};

R_{B} and R_{D} together can optionally form a cyclic structure;

R_{C} and R_{D} together can optionally form a cyclic structure; and
R_{E} is a substituted, unsubstituted, branched or unbranched C₁₋₂₀ aliphatic or a substituted, unsubstituted, branched or unbranched C₁₋₂₀ heteroaliphatic.

In certain particular embodiments, the lipidoid is of Formula (VI):

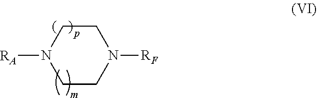

or a pharmaceutically acceptable salt thereof, wherein:

p is an integer between 1 and 3, inclusive;

m is an integer between 1 and 3, inclusive;

R_{A} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

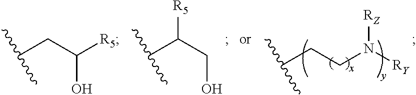

R_{F} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

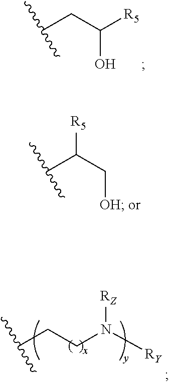

each occurrence of R₅ is independently hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; or substituted or unsubstituted heteroaryl;

wherein, at least one of R_{A}, R_{F}, R_{Y}, and R_{Z} is

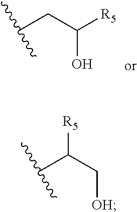

each occurrence of x is an integer between 1 and 10, inclusive;

each occurrence of y is an integer between 1 and 10, inclusive;

each occurrence of R_{Y} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

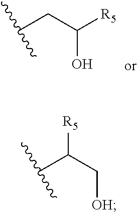

each occurrence of R_{Z} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

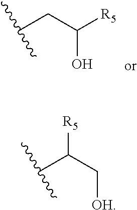

In certain embodiments of Formula (VI), p is 1. In certain embodiments, m is 1. In certain embodiments, p and m are both 1. In certain embodiments, R_{F} is

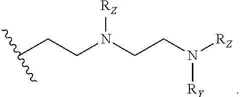

In certain embodiments, R_{A} is

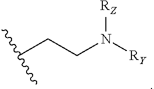

In certain embodiments, the composition comprises an aminoalcohol lipidoid selected from C14-120, C16-120, C14-98, C14-113, C14-96, C12-200, C12-205, C16-96, C12-111, and C12-210 (see U.S. Pat. No. 8,450,298 and PCT Publ. No. WO 2010/053572, referenced above).

In certain particular embodiments, the aminoalcohol lipidoid is C12-200:

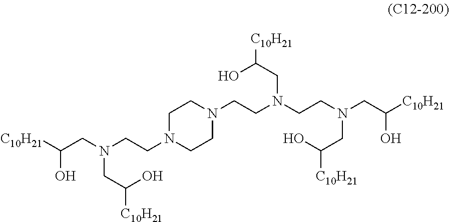

In certain particular embodiments, the lipidoid is of Formula (VII):

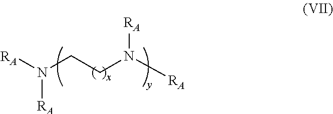

or a pharmaceutically acceptable salt thereof, wherein:

each occurrence of R_{A} is independently hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

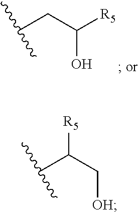

wherein at least one R_{A} is

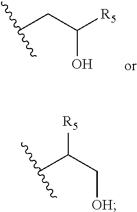

each occurrence of R₅ is independently hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; or substituted or unsubstituted heteroaryl;

each occurrence of x is an integer between 1 and 10, inclusive; and

each occurrence of y is an integer between 1 and 10, inclusive.

In certain embodiments, a composition described herein further comprises an amine-containing lipidoid as described in WO 2014/028847.

In certain embodiments, the amine-containing lipidoid is of Formula (VIII):

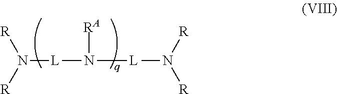

or a pharmaceutically acceptable salt thereof, wherein:

each L is, independently, branched or unbranched C₁₋₆ alkylene, wherein L is optionally substituted with one or more fluorine radicals;

each R^{A} is, independently, branched or unbranched C₁₋₆ alkyl, C₃₋₇ cycloalkyl, or branched or unbranched C₄₋₁₂ cycloalkylalkyl, wherein R^{A} is optionally substituted with one or more fluorine radicals;

each R is, independently, hydrogen or —CH₂CH₂C(═O)OR^{B};

each R^{B} is, independently, C₁₀₋₁₄ alkyl, wherein R^{B} is optionally substituted with one or more fluorine radicals; and

q is 1, 2, or 3;

provided that at least three R groups are —CH₂CH₂C(═O)OR^{B};

provided that the compound is not

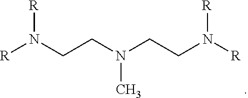

In certain embodiments, a composition described herein further comprises a polyamine-fatty acid derived lipidoid as described in WO 2016/004202.

In certain embodiments, the amine-containing lipidoid is of Formula (IX):

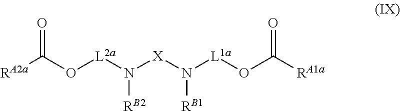

or a pharmaceutically acceptable salt, wherein:

X is substituted or unsubstituted alkylene, substituted or unsubstituted alkenylene, substituted or unsubstituted alkynylene, substituted or unsubstituted heteroalkylene, substituted or unsubstituted heteroalkenylene, substituted or unsubstituted heteroalkynylene, substituted or unsubstituted carbocyclylene, substituted or unsubstituted heterocyclylene, substituted or unsubstituted arylene, substituted or unsubstituted heteroarylene, a divalent moiety of the formula:

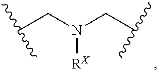

or a combination thereof, wherein each instance of R^{X} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group, or a moiety of the formula:

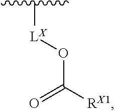

or R^{B1} and an instance of R^{X} are joined to form a substituted or unsubstituted, heterocyclic ring or a substituted or unsubstituted, heteroaryl ring, or R^{B2} and an instance of R^{X} are joined to form a substituted or unsubstituted, heterocyclic ring or a substituted or unsubstituted, heteroaryl ring, wherein:

each instance of L^{X} is independently substituted or unsubstituted alkylene or substituted or unsubstituted heteroalkylene; and

each instance of R^{X1} is independently substituted or unsubstituted, C₄₋₃₀ alkyl, substituted or unsubstituted, C₄₋₃₀ alkenyl, or substituted or unsubstituted, C₄₋₃₀ alkynyl;

L^{1a} is substituted or unsubstituted alkylene or substituted or unsubstituted heteroalkylene;

R^{A1a} is substituted or unsubstituted, C₄₋₃₀ alkyl, substituted or unsubstituted, C₄₋₃₀ alkenyl, or substituted or unsubstituted, C₄₋₃₀ alkynyl;

R^{B1} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group, or a moiety of the formula:

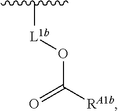

wherein L^{1b} is substituted or unsubstituted alkylene or substituted or unsubstituted heteroalkylene, and R^{A1b} is substituted or unsubstituted, C₄₋₃₀ alkyl, substituted or unsubstituted, C₄₋₃₀ alkenyl, or substituted or unsubstituted, C₄₋₃₀ alkynyl;

L^{2a} is substituted or unsubstituted alkylene or substituted or unsubstituted heteroalkylene;

R^{A2a} is substituted or unsubstituted, C₄₋₃₀ alkyl, substituted or unsubstituted, C₄₋₃₀ alkenyl, or substituted or unsubstituted, C₄₋₃₀ alkynyl; and

R^{B2} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group, or a moiety of the formula:

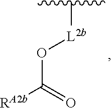

wherein L^{2b} is substituted or unsubstituted alkylene or substituted or unsubstituted heteroalkylene, and R^{A2b} is substituted or unsubstituted, C₄₋₃₀ alkyl, substituted or unsubstituted, C₄₋₃₀ alkenyl, or substituted or unsubstituted, C₄₋₃₀ alkynyl; or

R^{B1} and R^{B2} are joined to form a substituted or unsubstituted, heterocyclic ring or a substituted or unsubstituted, heteroaryl ring.

In certain embodiments, a composition described herein further comprises an amino acid-, peptide- or polypeptide-lipid as described in WO 2013/063468. In certain embodiments, the amine-containing lipidoid is of Formula (X):

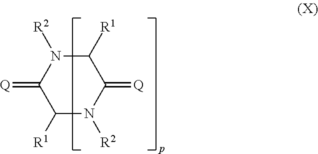

or a pharmaceutically acceptable salt, wherein:

p is an integer of between 1 and 9, inclusive;

each instance of Q is independently O, S, or NR^{Q}, wherein R^{Q} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), (iii);

each instance of R¹ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, halogen, —OR^{A1}, —N(R^{A1})₂, —SR^{A1}; wherein each occurrence of R^{A1} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to an sulfur atom, a nitrogen protecting group when attached to a nitrogen atom, or two R^{A1} groups are joined to form an optionally substituted heterocyclic or optionally substituted heteroaryl ring;

or at least one instance of R¹ is a group of formula:

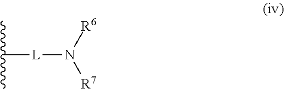

wherein L is an optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, or optionally substituted heteroarylene, and

R⁶ and R⁷ are each independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group;

each instance of R² is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), or (iii); and

Formulae (i), (ii), and (iii) are:

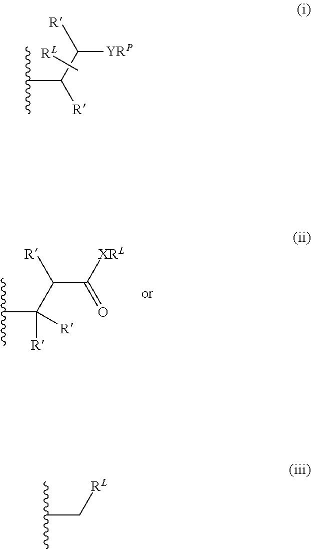

wherein:

each instance of R′ is independently hydrogen or optionally substituted alkyl;

X is O, S, NR^{X}, wherein R^{X} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;

Y is O, S, NR^{Y}, wherein R^{Y} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;

R^{P} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom; and

R^{L} is optionally substituted C₁₋₅₀ alkyl, optionally substituted C₂₋₅₀ alkenyl, optionally substituted C₂₋₅₀ alkynyl, optionally substituted heteroC₁₋₅₀ alkyl, optionally substituted heteroC₂₋₅₀ alkenyl, optionally substituted heteroC₂₋₅₀ alkynyl, or a polymer;

provided that at least one instance of R^{Q}, R², R⁶, or R⁷ is a group of the formula (i), (ii), or (iii).

In certain particular embodiments, the amino acid-, peptide- or polypeptide-lipid has the formula:

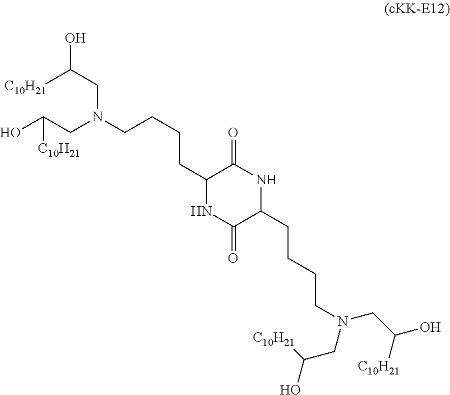

In certain particular embodiments, a composition as described herein can be formulated with C12-200 containing lipid nanoparticles. In some embodiments, the C12-200 is present in a molar percentage of about 1.0% to about 60.0%, about 10.0% to 40.0%, or about 20.0% to about 50.0% of the total composition. In some embodiments, the composition comprises C12-200 in a concentration of about 5.0%, about 7.5%, about 10.0%, about 12.5%, about 15.0%, about 17.5%, about 20.0%, about 20.5%, about 21.0%, about 21.5%, about 22.0%, about 22.5%, about 23.0%, about 23.5%, about 24.0%, about 24.5%, about 25.0%, about 25.5%, about 26.0%, about 26.5%, about 27.0%, about 27.5%, about 28.0%, about 28.5%, about 29.0%, about 29.5%, about 30.0%, about 30.5%, about 31.0%, about 31.5%, about 32.0%, about 32.5%, about 33.0%, about 33.5%, about 34.0%, about 34.5%, about 35.0%, about 35.5%, about 36.0%, about 36.5%, about 37.0%, about 37.5%, about 38.0%, about 38.5%, about 39.0%, about 39.5%, about 40.0%, about 40.5%, about 41.0%, about 41.5%, about 42.0%, about 42.5%, about 43.0%, about 43.5%, about 44.0%, about 44.5%, about 45.0%, about 45.5%, about 46.0%, about 46.5%, about 47.0%, about 47.5%, about 48.0%, about 48.5%, about 49.0%, about 49.5%, about 50.0%, about 50.5%, about 51.0%, about 52.0%, about 53.0%, about 54.0%, about 55.0%, about 56.0%, about 57.0%, about 58.0%, about 59.0% or about 60.0& by mole of the total composition. In certain embodiments, the composition comprises about 50.0% by mole C12-200.

In some embodiments, the lipid nanoparticles can also include one or more auxiliary lipids (also referred to herein as “co-lipids”) including, but not limited to, neutral lipids, amphipathic lipids, PEG-containing lipids, anionic lipids, and sterols.

In some embodiments, the lipid nanoparticles further comprise one or more neutral lipids. Neutral lipids, when present, can be any of a number of lipid species, which exist either in an uncharged or neutral zwitterionic form at physiological pH. Such lipids include, for example, diacylphosphatidylcholine, diacylphosphatidylethanolamine, ceramide, sphingomyelin, dihydrosphingomyelin, cephalin, and cerebrosides. In some embodiments, the neutral lipid component is a lipid having two acyl groups (e.g., diacylphosphatidylcholine and diacylphosphatidylethanolamine). In some embodiments, the neutral lipid comprises saturated fatty acids with carbon chain lengths in the range of C₁₀ to C₂₀, inclusive, In some embodiments, the neutral lipid includes mono- or di-unsaturated fatty acids with carbon chain lengths in the range of C₁₀ to C₂₀, inclusive. Suitable neutral lipids include, but are not limited to, DPPC (Dipalmitoyl phosphatidylcholine), POPC (Palmitoyl-Oleoyl Phosphatidyl Cholin), DOPE (1,2-dioleoyl-sn-glycero-3-phosphoethanolamine), DSPC (disteroylphosphatidyl choline), egg L-alpha-phosphatidylcholine (EPC); 1,2-dipalmitoyl-sn-glycero-3-phosphoethanolamine (DPPE); and SM (Sphingomyelin). In some embodiments, the neutral lipid is DSPC (disteroylphosphatidyl choline). In some embodiments, the composition comprises DSPC at about 1.0% to about 20.0%, or from about 5.0% to about 10.0% by mole of the total composition. In some embodiments, the composition comprises DSPC at about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, about 11.0%, about 11.5%, about 12.0%, about 12.5%, about 13.0%, about 13.5%, about 14.0%, about 14.5%, about 15.0%, about 15.5%, about 16.0%, about 16.5%, about 17.0%, about 17.5%, about 18.0%, about 18.5%, about 19.0% about 19.5% or about 20.0% by mole of the total composition. In some embodiments, composition comprises about 10% DSPC by mole.

In some embodiments, the lipid nanoparticles further comprise one or more anionic lipids. Anionic lipids are lipids that carry a net negative charge at physiological pH. Anionic lipids, when used in combination with cationic lipids, can reduce the overall surface charge of lipid particles, and/or introduce pH-dependent disruption of lipid structures, facilitating the release of therapeutic agents formulated in the lipid particles (e.g., siRNA molecules). Anionic lipids can include, but are not limited to, fatty acids (e.g., oleic, linoleic, linolenic acids); cholesteryl hemisuccinate (CHEMS); 1,2-di-0-tetradecyl-sn-glycero-3-phospho-(1′-rac-glycerol) (Diether PG); 1,2-dimyristoyl-sn-glycero-3-phospho-(1′-rac-glycerol) (sodium salt); 1,2-dimyristoyl-sn-glycero-3-phospho-L-serine (sodium salt); 1-hexadecanoyl,2-(9Z,12Z)-octadecadienoyl-sn-glycero-3-phosphate; 1,2-dioleoyl-sn-glycero-3-[phospho-rac-(1-glycerol)] (DOPG); dioleoylphosphatidic acid (DOPA); 1,2-dioleoyl-sn-glycero-3-phospho-L-serine (DOPS); and derivatives thereof. Other examples of suitable anionic lipids include, but are not limited to: fatty acids, such as oleic, linoleic, and linolenic acids; and cholesteryl hemisuccinate. Such lipids can be used alone or in combination, for a variety of purposes, such as to attach ligands to the liposome surface.

The lipid nanoparticle can also include one or more lipids capable of reducing aggregation. Examples of lipids that reduce aggregation of particles during formulation include PEG lipids (e.g., DMG-PEG (1,2-Dimyristoyl-sn-glycerol, methoxypolyethylene glycol-PEG), DMA-PEG (poly(ethylene glycol)-dimethacrylate-PEG) and DMPE-PEG550 (1,2-dimyristoyl-sn-glycero-3-phosphoethanolamine-N-[methoxy(polyethylene glycol)-550]), PEG), monosialoganglioside Gml, and polyamide oligomers (PAO), such as those described in U.S. Pat. No. 6,320,017. The lipid nanoparticles can include DMPE-PEG2000 or DMG-PEG which could be substituted with DMPE-PEG2000 in any of the formulations taught herein. Other suitable PEG lipids include, but are not limited to, PEG-modified phosphatidylethanolamine and phosphatidic acid, PEG-ceramide conjugates (e.g., PEG-CerC₁₄ or PEG-CerC₂₀) (such as those described in U.S. Pat. No. 5,820,873), PEG-modified dialkylamines and PEG-modified 1,2-diacyloxypropan-3-amines, PEG-modified diacylglycerols and dialkylglycerols, mPEG (mw2000)-diastearoylphosphatidylethanolamine (PEG-DSPE).

In some embodiments, a lipid capable of reducing aggregation is DMPE-PEG2000 or DMG-PEG (1,2-Dimyristoyl-sn-glycerol, methoxypolyethylene glycol, PEG). In some embodiments, the compositions comprises about 0.1% to about 5.0% DMPE-PEG2000 or DMG-PEG by mole (i.e., about 0.1% to about 5.0% DMPE-PEG2000 or 0.1% to about 5.0% DMG-PEG) or from about 0.5% to 2.0% DMPE-PEG2000 or DMG-PEG by mole. In some embodiments, the composition comprises about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% DMPE-PEG2000 or DMG-PEG by mole in the total composition. In some embodiments, the composition comprises about 1.5% DMPE-PEG2000 or DMG-PEG by mole.

In some embodiments, the lipid nanoparticle further comprises a sterol. In some embodiments, the sterol is cholesterol. In some embodiments, the composition comprises from about 10.0% to about 50.0% cholesterol by mole, or about 15.0% to about 40.0% cholesterol by mole. In some embodiments, the composition comprises about 10.0%, about 11.0%, about 11.5%, about 12.0%, about 12.5%, about 13.0%, about 13.5%, about 14.0%, about 14.5%, about 15.0%, about 15.5%, about 16.0%, about 16.5%, about 17.0%, about 17.5%, about 18.0%, about 18.5%, about 19.0%, about 19.5%, about 20.0%, about 20.5%, about 21.0%, about 21.5%, about 22.0%, about 22.5%, about 23.0%, about 23.5%, about 24.0%, about 24.5%, about 25.0%, about 25.5%, about 26.0%, about 26.5%, about 27.0%, about 27.5%, about 28.0%, about 28.5%, about 29.0%, about 29.5%, about 30.0%, about 30.5%, about 31.0%, about 31.5%, about 32.0%, about 32.5%, about 33.0%, about 33.5%, about 34.0%, about 34.5%, about 35.0%, about 35.5%, about 36.0%, about 36.5%, about 37.0%, about 37.5%, about 38.0%, about 38.5%, about 39.0%, about 39.5% or about 40.0% cholesterol by mole. In some embodiments, the composition comprises about 38.5% cholesterol by mole.

The ratio of PEG in the LNP formulations can be increased or decreased and/or the carbon chain length of the PEG lipid can be modified from C14 to C18 to alter the pharmacokinetics and/or biodistribution of the LNP formulations.

In some embodiments, the lipid nanoparticles described herein further comprise one or more compounds that are capable of enhancing the cellular uptake or cytosolic distribution of the lipid nanoparticle and/or its encapsulated composition (e.g., gene silencing agent, siRNA molecule, peptide, etc.). Compounds that can enhance the cellular uptake can include levodopa, naphazoline hydrochloride, acetohexamide, niclosamide, diprophylline, and isoxicam, or a combination thereof. Compounds that can enhance the cytosolic distribution can include azaguanine-8, isoflupredone acetate, chloroquine, trimethobenzamide, hydrochloride, isoxsuprine hydrochloride, and diphemanil methylsulfate, or a combination thereof.

In some embodiments, the lipid nanoparticles comprise lipid bilayers encapsulating one or more agents encompassed by the present invention, such as siRNA molecules sufficiently complementary to the mRNA transcription product of at least one biomarker described herein. In some embodiments, the lipid nanoparticles are formulated to facilitate an uptake into cells. In some embodiments, the lipid nanoparticles are formulated to facilitate uptake into monocytes, dendritic cells, and/or macrophages.

The lipid nanoparticle can, in some aspects, further comprise additional agents. In some embodiments, the lipid nanoparticle further comprises one or more antioxidants. Without wishing to be bound by any particular theory, the antioxidant can help stabilize the lipid nanoparticle and prevent, decrease, and/or inhibit degradation of the cationic lipids and/or active agents encapsulated in the lipid nanoparticle. In some embodiments, the antioxidant is a hydrophilic antioxidant, a lipophilic antioxidant, a metal chelator, a primary antioxidant, a secondary antioxidant, or salts or mixtures thereof. In some embodiments, the antioxidant comprises EDTA, or a salt thereof. In some embodiments, the lipid nanoparticle furhter comprises EDTA in combination with one, two, three, four, five, six, seven, eight, or more additional antioxidants (e.g., primary antioxidants, secondary antioxidants, or other metal chelators). Examples of antioxidants include, but are not limited to, hydrophilic antioxidants, lipophilic antioxidants, and mixtures thereof. Non-limiting examples of hydrophilic antioxidants include chelating agents (e.g., metal chelators) such as ethylenediaminetetraacetic acid (EDTA), citrate, ethylene glycol tetraacetic acid (EGTA), 1,2-bis(o-aminophenoxy)ethane-N,N,N′,N′-tetraacetic acid (BAPTA), diethylene triamine pentaacetic acid (DTPA), 2,3-dimercapto-1-propanesulfonic acid (DMPS), dimercaptosuccinic acid (DMSA), cc-lipoic acid, salicylaldehyde isonicotinoyl hydrazone (SIR), hexyl thioethylamine hydrochloride (HTA), desferrioxamine, salts thereof, and mixtures thereof. Additional hydrophilic antioxidants include ascorbic acid, cysteine, glutathione, dihydrolipoic acid, 2-mercaptoethane sulfonic acid, 2-mercaptobenzimidazole sulfonic acid, 6-hydroxy-2,5,7,8-tetramethylchroman-2-carboxylic acid, sodium metabisulfite, salts thereof, and mixtures thereof. Non-limiting examples of lipophilic antioxidants include vitamin E isomers such as α-, β-, γ-, and δ-tocopherols and α-, β-, γ-, and δ-tocotrienols; polyphenols such as 2-tert-butyl-4-methyl phenol, 2-tert-butyl-5-methyl phenol, and 2-tert-butyl-6-methyl phenol; butylated hydroxyanisole (BHA) (e.g., 2-teri-butyl-4-hydroxyanisole and 3-tert-butyl-4-hydroxyanisole); butylhydroxytoluene (BHT); tert-butylhydroquinone (TBHQ); ascorbyl palmitate; rc-propyl gallate; salts thereof; and mixtures thereof.

In some embodiments, the lipid-based particles formulated for delivery of one or more agents (e.g., gene silencing agents, siRNA molecules, peptides) are selected from lipid vectors, liposomes, lipoplexes, lipid nanoparticles, and micelles. In some embodiments, the lipid-based particle is a pH-sensitive nanoparticle. Such pH-sensitive nanoparticles (PNSDS), which are positive-charge-free nanocarriers comprising siRNA chemically cross-linked with multi-armed poly(ethylene glycol) carriers via acid-labile acetal linkers, can be beneficial for the delivery of siRNA molecules (Tang et al., SiRNA Crosslinked Nanoparticles for the Treatment of Inflammation-induced Liver Injury, Advanced Science, 2016, 4(2), e1600228).

In some embodiments, the lipid nanoparticle further comprises one or more C12-200 aminoalcohol lipids. In some embodiments, the lipid nanoparticle comprises from about 40.0% to about 50.0% C12-200 by mole. In some embodiments, the lipid nanoparticle comprises from about 5.0% to about 10.0% DSPC by mole. In some embodiments, the lipid nanoparticle comprises from about 1.0% to about 2.0% DMG-PEG by mole. In some embodiments, the lipid nanoparticle comprises from about 20.0% to about 40.0% cholesterol by mole. In some embodiments, the lipid nanoparticle comprises 50% C12-200, 10.0% DSPC, 1.5% DMG-PEG, and 38.5% cholesterol by mole.

In some embodiments, the total siRNA molecule moles with respect to the total lipid moles within the formulation ranges from about 1:5 to about 1:20. In some embodiments, the total siRNA molecule moles with respect to the total lipid moles is about 1:5, about 1:6, about 1:7, about 1:8, about 1:9, about 1:10, about 1:11, about 1:12, about 1:13, about 1:14, about 1:15, about 1:16, about 1:17, about 1:18, about 1:18, about 1:19, or about 1:20. In some embodiments, the total siRNA molecule moles with respect to the total lipid moles is about 1:9.

In some embodiments, the lipid nanoparticle (LNP) is formulated to encapsulate an agent, such as an siRNA, using a spontaneous vesicle formation formulation procedure as previously described in Semple et al. (2010) Nat. Biotechnol. 28172-28176.

In some embodiments, the total concentration of one or more agents encompassed by the present invention, such as siRNA molecules that are sufficiently complementary to the mRNA transcription product of at least one biomarker described herein in the formulation is about 0.001 mg/ml to about 100 mg/ml, about 0.01 mg/ml to about 10 mg/ml, or about 0.1 mg/ml to about 20 mg/ml. In some embodiments, the total concentration of two or more, three or more, four or more, five or more, or all six siRNA molecules is about 0.001 mg/ml to about 100 mg/ml, about 0.01 mg/ml to about 10 mg/ml, or about 0.1 mg/ml to about 20 mg/ml.

In some embodiments, the lipid nanoparticles (LNPs) ranging in size from about 40 to about 200 nm, or from about 50 nm to about 100 nm. In some embodiments, the lipid nanoparticle is about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 110 nm, about 120 nm, about 130 nm, about 140 nm, about 150 nm, about 160 nm, about 170 nm, about 180 nm, or about 200 nm in size. In some embodiments, the lipid nanoparticle is about 80 nm in size.

In accordance with the present invention, the formulations as described herein are stable. The term “stable,” as used herein, means remaining in a state or condition that is suitable for administration to a patient. In some embodiments, the formulations are substantially pure. As used herein, “substantially pure” means that the active ingredient (e.g., the siRNA molecules sufficiently complementary to the mRNA transcription product of at least one biomarker described herein) is the predominant species present in the formulation. In some embodiments, a substantially pure composition comprises a composition that is more than 80% comprised of macromolecular species (e.g., active agents, gene silencing agents, siRNA molecules, additional agents (e.g., antioxidants)). In some embodiments, the substantially pure composition comprises a composition that is more than 85%, 90%, 95%, 96%, 97%, 98%, or 99% comprised of macromolecular species. In some embodiments, the one or more active agents are purified to essential homogeneity (i.e., contaminant species cannot be detected in the composition by conventional detection methods), wherein the composition consists essentially of a single macromolecular species.

Other nanoparticles can be used as delivery vehicles of the agents and compositions described herein. In some embodiments, the nanoparticles comprises chemically and/or enzymatically modified lipoproteins (e.g., apolipoproteins as described in U.S. Pat. Publ. No. 2011/0256224). In some embodiments, the nanoparticles comprise other lipoprotein-based nanoparticles, such as HDL, HDL-like lipoprotein particles, or synthetic HDL-like particles (See, e.g., U.S. Pat. Publ. No. 2009/0110739 and U.S. Pat. No. 7,824,709).

In some embodiments, nanoparticles with increased macrophage targeted delivery are used to encapsulate a composition as described herein. In some embodiments, the nanoparticle is a GP nanoparticle comprising 1,3-D-glucan (Soto et al. (2012) J. Drug. Deliv. e143524), or a mannosylated chitosan (MCS) nanoparticle (Peng et al. (2015) J. Nanosci. Nanotechnol. 15:2619-2627).

The nanoparticle formulations can be a carbohydrate nanoparticle comprising a carbohydrate carrier. As a non-limiting example, the carbohydrate carrier can include, but is not limited to, an anhydride-modified phytoglycogen or glycogen-type material, phtoglycogen octenyl succinate, phytoglycogen beta-dextrin, anhydride-modified phytoglycogen beta-dextrin. (see, e.g., PCT Publ. No. WO 2012/109121).

In some embodiments, lipid nanoparticles can be engineered to alter the surface properties of particles so the lipid nanoparticles can penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles can be removed from the mucosa tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. (2007) Proc. Natl. Acad. Sci. U.S.A. 104:1482-1487; Lai et al. (2009) Adv Drug Deliv Rev. 61:158-171). The transport of nanoparticles can be determined using rates of permeation and/or fluorescent microscopy techniques including, but not limited to, fluorescence recovery after photo bleaching (FRAP) and high resolution multiple particle tracking (MPT). As a non-limiting example, compositions which can penetrate a mucosal barrier can be made as described in U.S. Pat. No. 8,241,670.

Lipid nanoparticle engineered to penetrate mucus can comprise a polymeric material (i.e., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material can be biodegradable and/or biocompatible. The polymeric material can additionally be irradiated. As a non-limiting example, the polymeric material can be gamma irradiated (see, e.g., PCT Publ. No. WO 2012/082165). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), and trimethylene carbonate, polyvinylpyrrolidone. The lipid nanoparticle can be coated or associated with a co-polymer such as, but not limited to, a block co-polymer, and (poly(ethylene glycol))-(poly(propylene oxide))-(poly(ethylene glycol)) triblock copolymer (see, e.g., U.S. Pat. Publ. Numbers 2012/0121718 and 2010/0003337; and U.S. Pat. No. 8,263,665). The co-polymer can be a polymer that is generally regarded as safe (GRAS) and the formation of the lipid nanoparticle can be in such a way that no new chemical entities are created. For example, the lipid nanoparticle can comprise poloxamers coating PLGA nanoparticles without forming new chemical entities which are still able to rapidly penetrate human mucus (Yang et al. (2011) Angew. Chem. Int. Ed. 50:2597-2600).

For example, LNPs encompassed by the present invention can comprise a PLGA-PEG block copolymer (see, e.g., U.S. Pat. Publ. No. 2012/0004293 and U.S. Pat. No. 8,236,330); a diblock copolymer of PEG and PLA or PEG and PLGA (see, e.g., U.S. Pat. No. 8,246,968); a multiblock copolymer (see, e.g., U.S. Pat. Nos. 8,263,665 and 8,287,910); a polyion complex comprising a non-polymeric micelle and the block copolymer (see, e.g., U.S. Pat. Publ. No. 2012/00768); or amine-containing polymer such as, but not limited to polylysine, polyethylene imine, poly(amidoamine) dendrimers, poly(beta-amino esters) (see, e.g., U.S. Pat. No. 8,287,849).

LNPs encompassed by the present invention can comprise one or more other polymer such as acrylic polymers. Acrylic polymers can include but are not limited to, acrylic acid, methacrylic acid and methacrylic acid copolymersx, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

LNPs encompassed by the present invention can comprise at least one degradable polyester which can contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters can include a PEG conjugation to form a PEGylated polymer. The LNPs can further include at least one targeting ligand. The targeting ligand can be any ligand known in the art such as, but not limited to, a monoclonal antibody (Kirpotin et al. (2006) Cancer Res. 66:6732-6740).

In some embodiments, compositions encompassed by the present invention can be formulated as a solid lipid nanoparticle. A solid lipid nanoparticle (SLN) can be spherical with an average diameter between 10 to 1000 nm. SLN possess a solid lipid core matrix that can solubilize lipophilic molecules and can be stabilized with surfactants and/or emulsifiers. In a further embodiment, the lipid nanoparticle can be a self-assembly lipid-polymer nanoparticle (see, e.g., Zhang et al. (2008) ACS Nano 2:1696-1702).

In some embodiments, agents encompassed by the present invention can be sustained release formulations, such as encapsulated into a nanoparticle or a rapidly eliminated nanoparticle and the nanoparticles or a rapidly eliminated nanoparticle can then be encapsulated into a polymer, hydrogel and/or surgical sealant described herein and/or known in the art. As a non-limiting example, the polymer, hydrogel or surgical sealant can be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.). In another embodiment, the nanoparticle can be encapsulated into any polymer known in the art which can form a gel when injected into a subject. As a non-limiting example, the nanoparticle can be encapsulated into a polymer matrix which can be biodegradable.

In some embodiments, compositions encompassed by the present invention can be formulated as controlled release nanoparticles. In one example, the nanoparticle formulation for controlled release and/or targeted delivery can further include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®). In another example, the controlled release and/or targeted delivery formulation can comprise at least one degradable polyester which can contain polycationic side chains. Degradable polyesters include, but are not limited to, poly(serine ester), poly(L-lactide-co-L-lysine), poly(4-hydroxy-L-proline ester), and combinations thereof.

In another embodiment, the degradable polyesters can include a PEG conjugation to form a PEGylated polymer.

In some embodiments, compositions encompassed by the present invention can be formulated as a lipoplex, such as, without limitation, the ATUPLEX™ system, the DACC system, the DBTC system and other conjugate-lipoplex technology from Silence Therapeutics (London, United Kingdom), STEMFECT™ from STEMGENT® (Cambridge, Mass.), and polyethylenimine (PEI) or protamine-based targeted and non-targeted delivery of therapeutic agents (Aleku et al. (2008) Cancer Res. 68: 9788-9798; Strumberg et al. (2012) Int. J Clin. Pharmacol. Ther. (2012) 50:76-78; Santel et al. (2006) Gene Ther. 13:1222-1234; Santel et al. (2006) Gene Ther. 13:1360-1370; Gutbier et al. (2010) Pulm. Pharmacol. Ther. 23:334-344; Kaufmann et al. (2010) Microvasc. Res. 80:286-293; Weide et al. (2009) J. Immunother. 32:498-507; Weide et al. (2008) J. Immunother. 31:180-188; Pascolo (2004) Exp. Opin. Biol. Ther. 4:1285-1294; Fotin-Mleczek et al. (2011) J Immunother. 34:1-15; Song et al. (2005) Nature Biotechnol. 23:709-717; Peer et al. (2007) Proc. Natl. Acad. Sci. U.S.A. 6:4095-4100; and deFougerolles (2008) Hum. Gene Ther. 19:125-132).

In some embodiments, therapeutic agents and compositions encompassed by the present invention can be encapsulated in, linked to and/or associated with synthetic nanocarriers. Synthetic nanocarriers include, but are not limited to, those described in International Pub. Nos. WO 2010/005740, WO 2010/030763, WO 2012/13501, WO 2012/149252, WO 2012/149255, WO 2012/149259, WO 2012/149265, WO 2012/149268, WO 2012/149282, WO 2012/149301, WO 2012/149393, WO 2012/149405, WO 2012/149411, and WO 2012/149454, and U.S. Pat. Publ. Numbers 2011/0262491, 2010/0104645, 2010/0087337, and 2012/0244222. In another embodiment, the synthetic nanocarrier formulations can be lyophilized, such as by methods described in PCT Publ. No. WO 2011/072218 and U.S. Pat. No. 8,211,473.

In some embodiments, the synthetic nanocarriers can contain reactive groups to release the conjugates described herein (see, e.g., PCT Publ. No. WO 2012/0952552 and U.S. Pat. Publ. No. 2012/0171229). In one embodiment, the synthetic nanocarriers can be formulated for targeted release. In one embodiment, the synthetic nanocarrier is formulated to release the therapeutic agents at a specified pH and/or after a desired time interval. As a non-limiting example, the synthetic nanoparticle can be formulated to release the conjugates after 24 hours and/or at a pH of 4.5 (see, e.g., PCT Publ. Numbers WO 2010/138193 and WO 2010/138194 and U.S. Pat. Publ. Numbers 2011/0020388 and 2011/0027217). In some embodiments, the synthetic nanocarriers can be formulated for controlled and/or sustained release of conjugates described herein. As a non-limiting example, the synthetic nanocarriers for sustained release can be formulated by methods known in the art, described herein and/or as described in PCT Publ. No. WO 2010/138192 and U.S. Pat. Publ. No. 2010/0303850.

In some embodiments, the nanoparticle can be optimized for oral administration. The nanoparticle can comprise at least one cationic biopolymer such as, but not limited to, chitosan or a derivative thereof. As a non-limiting example, the nanoparticle can be formulated by the methods described in U.S. Pat. Publ. No. 20120282343.

In some embodiments, agents encompassed by the present invention can also be formulated using natural and/or synthetic polymers. Non-limiting examples of polymers which can be used for drug delivery include, but are not limited to, DYNAMIC POLYCONJUGATE® (Arrowhead Research Corp., Pasadena, Calif.) formulations from MIRUS® Bio (Madison, Wis.) and Roche Madison (Madison, Wis.), PHASERX™ polymer formulations such as, without limitation, SMARTT POLYMER TECHNOLOGY™ (Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers, RONDEL™ (RNAi/Oligonucleotide Nanoparticle Delivery) polymers (Arrowhead Research Corporation, Pasadena, Calif.) and pH responsive co-block polymers such as, but not limited to, PHASERX™ (Seattle, Wash.). For example, agents and compositions encompassed by the present invention can be formulated in a pharmaceutical compound including a poly(alkylene imine), a biodegradable cationic lipopolymer, a biodegradable block copolymer, a biodegradable polymer, or a biodegradable random copolymer, a biodegradable polyester block copolymer, a biodegradable polyester polymer, a biodegradable polyester random copolymer, a linear biodegradable copolymer, PAGA, a biodegradable cross-linked cationic multi-block copolymer or combinations thereof.

The polymers used in the present invention can have undergone processing to reduce and/or inhibit the attachment of unwanted substances such as, but not limited to, bacteria, to the surface of the polymer. The polymer can be processed by methods known and/or described in the art and/or described in PCT Publ. No. WO 2011/50467.

Nanoparticles can contain one or more polymers. Polymers can contain one more of the following polyesters: homopolymers including glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA,” and caprolactone units, such as poly(ε-caprolactone), collectively referred to herein as “PCL,” and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA,” and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers.” In certain embodiments, the PEG region can be covalently associated with polymer to yield “PEGylated polymers” by a cleavable linker.

The nanoparticles can contain one or more hydrophilic polymers. Hydrophilic polymers include cellulosic polymers such as starch and polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol); polyoxazoline; and copolymers thereof.

The nanoparticles can contain one or more hydrophobic polymers. Examples of suitable hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof.

In certain embodiments, the hydrophobic polymer is an aliphatic polyester. In some embodiments, the hydrophobic polymer is poly(lactic acid), poly(glycolic acid), or poly(lactic acid-co-glycolic acid).

The nanoparticles can contain one or more amphiphilic polymers. Amphiphilic polymers can be polymers containing a hydrophobic polymer block and a hydrophilic polymer block. The hydrophobic polymer block can contain one or more of the hydrophobic polymers above or a derivative or copolymer thereof. The hydrophilic polymer block can contain one or more of the hydrophilic polymers above or a derivative or copolymer thereof. In some embodiments the amphiphilic polymer is a di-block polymer containing a hydrophobic end formed from a hydrophobic polymer and a hydrophilic end formed of a hydrophilic polymer. In some embodiments, a moiety can be attached to the hydrophobic end, to the hydrophilic end, or both. The particle can contain two or more amphiphilic polymers.

The polymer can also include but is not limited to, polyethenes, polyethylene glycol (PEG), poly(l-lysine) (PLL), PEG grafted to PLL, cationic lipopolymer, biodegradable cationic lipopolymer, polyethylenimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

The polymers can be a cross linkable polyester. Cross linkable polyesters include those known in the art and described in U.S. Pat. Publ. No. 2012/0269761.

The nanoparticles can contain one or more biodegradable polymers. Biodegradable polymers can include polymers that are insoluble or sparingly soluble in water that are converted chemically or enzymatically in the body into water-soluble materials. Biodegradable polymers can include soluble polymers crosslinked by hydolyzable cross-linking groups to render the crosslinked polymer insoluble or sparingly soluble in water.

Biodegradable polymers can include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose such as methyl cellulose and ethyl cellulose, hydroxyalkyl celluloses such as hydroxypropyl cellulose, hydroxy-propyl methyl cellulose, and hydroxybutyl methyl cellulose, cellulose ethers, cellulose esters, nitro celluloses, cellulose acetate, cellulose propionate, cellulose acetate butyrate, cellulose acetate phthalate, carboxylethyl cellulose, cellulose triacetate, cellulose sulphate sodium salt, polymers of acrylic and methacrylic esters such as poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene imines), poly(caprolactones), poly(hydroxyalkanoates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof. In some embodiments the particle contains biodegradable polyesters or polyanhydrides such as poly(lactic acid), poly(glycolic acid), and poly(lactic-co-glycolic acid).

Degradable polyesters can contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters can include a PEG conjugation to form a PEGylated polymer.

The biodegradable cationic lipopolymer can be made by methods known in the art, such as those described in U.S. Pat. No. 6,696,038 and U.S. Pat. Publ. Numbers 2003/0073619 and 2004/0142474. The poly(alkylene imine) can be made using methods known in the art, such as those described in U.S. Pat. Publ. No. 2010/0004315. The biodegradable polymer, biodegradable block copolymer, the biodegradable random copolymer, biodegradable polyester block copolymer, biodegradable polyester polymer, or biodegradable polyester random copolymer can be made using methods known in the art, such as those described in U.S. Pat. Nos. 6,517,869 and 6,267,987. The linear biodegradable copolymer can be made using methods known in the art, such as those described in U.S. Pat. No. 6,652,886. The PAGA polymer can be made using methods known in the art, such as those described in U.S. Pat. No. 6,217,912. The PAGA polymer can be copolymerized to form a copolymer or block copolymer with polymers such as but not limited to, poly-L-lysine, polyarginine, polyornithine, histones, avidin, protamines, polylactides and poly(lactide-co-glycolides). The biodegradable cross-linked cationic multi-block copolymers can be made using methods known in the art, such as those described in U.S. Pat. No. 8,057,821 and U.S. Pat. Publ. No. 2012/009145. For example, the multi-block copolymers can be synthesized using linear polyethylenimine (LPEI) blocks which have distinct patterns as compared to branched polyethyleneimines.

The polymers described herein can be conjugated to a lipid-terminating PEG. As a non-limiting example, PLGA can be conjugated to a lipid-terminating PEG forming PLGA-DSPE-PEG. As another non-limiting example, PEG conjugates for use according to the present invention are described in PCT Publ. No. WO 2008/103276. The polymers can be conjugated using a ligand conjugate such as, but not limited to, conjugates described in U.S. Pat. No. 8,273,363.

Polymer nanoparticles can also comprise chitosan. The chitosan formulation includes a core of positively charged chitosan and an outer portion of negatively charged substrate (see, e.g., U.S. Pat. Publ. No. 2012/0258176). Chitosan includes, but is not limited to N-trimethyl chitosan, mono-N-carboxymethyl chitosan (MCC), N-palmitoyl chitosan (NPCS), EDTA-chitosan, low molecular weight chitosan, chitosan derivatives, or combinations thereof.

Polymer nanoparticles can also comprise PLGA. The PLGA formulations can include, but are not limited to, PLGA injectable depots (e.g., ELIGARD® which is formed by dissolving PLGA in 66% N-methyl-2-pyrrolidone (NMP) and the remainder being aqueous solvent and leuprolide. Once injected, the PLGA and leuprolide peptide precipitates into the subcutaneous space. In other examples, PLGA microspheres can be formulated by preparing the PLGA microspheres with tunable release rates (e.g., days and weeks) and encapsulating the active agents in the PLGA microspheres while maintaining the integrity of the agent during the encapsulation process.

In some embodiments, Evac, which are non-biodegradable, biocompatible polymers used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; and catheters), can be used. Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C. PEG-based surgical sealants comprise two synthetic PEG components mixed in a delivery device which can be prepared in one minute, seals in 3 minutes and is reabsorbed within 30 days. GELSITE® and natural polymers are capable of in-situ gelation at the site of administration. They have been shown to interact with protein and peptide therapeutic candidates through ionic interaction to provide a stabilizing effect.

Other representative examples of polymer nanoparticles useful according to the present invention include the polymeric compound of PEG grafted with PLL as described in U.S. Pat. No. 6,177,274, as well as suspensions in a solution or medium with a cationic polymer, in a dry pharmaceutical composition or in a solution that is capable of being dried as described in U.S. Pat. Publ. Numbers 2009/0042829 and 2009/0042825.

A polyamine derivative can be used to deliver therapeutic agents and compositions encompassed by the present invention or to treat and/or prevent a disease or to be included in an implantable or injectable device (U.S. Pat. Publ. No. 2010/0260817). As a non-limiting example the agents encompassed by the present invention can be delivered using a polyamide polymer comprising a 1,3-dipolar addition polymer prepared by combining a carbohydrate diazide monomer with a dilkyne unite comprising oligoamines (U.S. Pat. No. 8,236,280).

Other polymers can include acrylic polymers, such as acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof; or amine-containing polymers such as, but not limited to polylysine, polyethylene imine, poly(amidoamine) dendrimers or combinations thereof; or a PEG-charge-conversional polymer (Pitella et al. (2011) Biomat. 32:3106-3114).

Polymer nanoparticle can further comprise a diblock copolymer. In one embodiment, the diblock copolymer can include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. In some embodiments, agents encompassed by the present invention can be formulated with a PLGA-PEG block copolymer (see, e.g., U.S. Pat. Publ. No. US 2012/0004293 and U.S. Pat. No. 8,236,330) or PLGA-PEG-PLGA block copolymers (see, e.g., U.S. Pat. No. 6,004,573). As a non-limiting example, the agents encompassed by the present invention can be formulated with a diblock copolymer of PEG and PLA or PEG and PLGA (see, e.g., U.S. Pat. No. 8,246,968).

In some embodiments, polymer nanoparticles can comprise a plurality of polymers such as, but not limited to hydrophilic-hydrophobic polymers (e.g., PEG-PLGA), hydrophobic polymers (e.g., PEG) and/or hydrophilic polymers (see, e.g., PCT Publ. No. WO 2012/0225129).

In some embodiments, polymer nanoparticles can be formulated as therapeutic nanoparticles. Therapeutic nanoparticles can be formulated by methods and polymers described herein and known in the art such as, but not limited to, PCT Publ. Numbers WO 2010/005740, WO 2010/030763, WO 2010/005721, WO 2010/005723, and WO 2012/054923, and U.S. Pat. Publ. Numbers 2011/0262491, 2010/0104645, 2010/0087337, 2010/0068285, 2011/0274759, 2010/0068286, and 2012/0288541, and U.S. Pat. Nos. 8,206,747; 8,293,276; 8,318,208; and 8,318,211. In some embodiments, therapeutic polymer nanoparticles can be identified by the methods described in U.S. Pat. Publ. No. 2012/0140790.

Polymer formulations can also be selectively targeted through expression of different ligands as exemplified by, but not limited by, folate, transferrin, and N-acetylgalactosamine (GalNAc) (Benoit et al. (2011) Biomacromol. 12:2708-2714; Rozema et al. (2007) Proc. Natl. Acad. Sci. U.S.A. 104:12982-12887; Davis (2009) Mol. Pharm. 6:659-668; Davis (2010) Nature 464:1067-1070).

In some embodiments, the polymer formulation encompassed by the present invention can be stabilized by contacting the polymer formulation, which can include a cationic carrier, with a cationic lipopolymer which can be covalently linked to cholesterol and polyethylene glycol groups. The polymer formulation can be contacted with a cationic lipopolymer using the methods described in U.S. Pat. Publ. No. 2009/0042829. The cationic carrier can include, but is not limited to, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B—[N—(N′,N′-Dimethylaminoethane)-carbamoyl]Cholesterol Hydrochloride (DC-Cholesterol HCl) diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride DODAC) and combinations thereof.

The conjugates encompassed by the present invention can be formulated in a polyplex of one or more polymers (see, e.g., U.S. Pat. Publ. Numbers 2012/0237565 and 2012/0270927). In one embodiment, the polyplex comprises two or more cationic polymers. The catioinic polymer can comprise a poly(ethylene imine) (PEI), such as linear PEI.

In some embodiments, other forms of nanoparticles can be used.

For example, agents and compositions encompassed by the present invention can be formulated as a nanoparticle using a combination of polymers, lipids, and/or other biodegradable agents, such as, but not limited to, calcium phosphate. Components can be combined in a core-shell, hybrid, and/or layer-by-layer architecture, to allow for fine-tuning of the nanoparticle so that delivery of the composition encompassed by the present invention. Biodegradable calcium phosphate nanoparticles in combination with lipids and/or polymers have been shown to deliver therapeutic agents in vivo. In one embodiment, a lipid coated calcium phosphate nanoparticle, which can also contain a targeting ligand such as anisamide, can be used to deliver the composition encompassed by the present invention (see, e.g., Li et al. (2010) J. Contr. Rel. 142:416-421; Li et al. (2012) J. Contr. Rel. 158:108-114; Yang et al. (2012) Mol. Ther. 20:609-615). This delivery system combines both a targeted nanoparticle and a component to enhance the endosomal escape, calcium phosphate, in order to improve delivery of the agent.

In some embodiments, the particles can be hydrophobic ion-pairing complexes or hydrophobic ioin-pairs formed by one or more conjugates described above and counterions.

In some embodiments, core-shell nanoparticles can be used for pharmaceutical formulations. The use of core-shell nanoparticles has additionally focused on a high-throughput approach to synthesize cationic cross-linked nanogel cores and various shells (Siegwart et al. (2011) Proc. Natl. Acad. Sci. U.S.A. 108:12996-13001). The complexation, delivery, and internalization of the polymeric nanoparticles can be precisely controlled by altering the chemical composition in both the core and shell components of the nanoparticle. For example, the core-shell nanoparticles can efficiently deliver a therapeutic agent to mouse hepatocytes after they covalently attach cholesterol to the nanoparticle. Core-shell nanoparticles for use with the composition encompassed by the present invention are described and can be formed by the methods described in U.S. Pat. No. 8,313,777.

Inorganic nanoparticles exhibit a combination of physical, chemical, optical and electronic properties and provide a highly multifunctional platform to image and diagnose diseases, to selectively deliver therapeutic agents, and to sensitive cells and tissues to treatment regiments. Not wishing to be bound to any theory, enhanced permeability and retention (EPR) effect of inorganic nanoparticle provides a basis for the selective accumulation of many high-molecular-weight drugs. Circulating inorganic nanoparticles preferentially accumulate at tumor sites and in inflamed tissues (Yuan et al. (1995) Cancer Res. 55:3752-3756) and remain lodged due to their low diffusivity (Pluen et al. (2001) Proc. Natl. Acad. Sci. U.S.A. 98:4628-4633. The size of the inorganic nanoparticles can be 10 nm-500 nm, 10 nm-100 nm, or 100 nm-500 nm. The inorganic nanoparticles can comprise metal (gold, iron, silver, copper, nickel, etc.), oxides (ZnO, TiO₂, Al₂O₃, SiO₂, iron oxide, copper oxide, nickel oxide, etc.), or semiconductor (CdS, CdSe, etc.). The inorganic nanoparticles can also be perfluorocarbon or FeCo.

Inorganic nanoparticles have high surface area per unit volume. Therefore, they can be loaded with therapeutic drugs and imaging agents at high densitives. A variety of methods can be used to load therapeutic drugs into/onto the inorganic nanoparticles, including but not limited to, colvalent bonds, electrostatic interactions, entrapment, and encapsulation. In addition to therapeutic agent drug loads, the inorganic nanoparticles can be funcationalized with targeting moieties, such as tumor-targeting ligands, on the surface. Formulating therapeutic agents with inorganic nanoparticles allows imaging, detection and monitoring of the therapeutic agents.

In some embodiments, agents and compositions encompassed by the present invention is hydrophobic and can be form a kinetically stable complex with gold nanoparticles funcationalized with water-soluble zwitterionic ligands (see, e.g., Kim et al. (2009) JACS 131:1360-1361).

Agents and compositions encompassed by the present invention can be formulated with gold nanoshells. As a non-limiting example, the compositions can be delivered with a temperature sensitive system comprising polymers and gold nanoshells and can be released photothermally (see, e.g., Sershen et al. (2000) J. Biomed. Mater. 51:293-298). Irradiation at 1064 nm was absorbed by the nanoshells and converted to heat, which led to the collapse of the hydrogen and release of the drug. Agents can also be encapsulated inside hollow gold nanoshells, such as by covalent bonding between agents and nanoparticles. Covalent attachment to gold nanoparticles can be achieved through a linker, such as a free thiol, amine or carboxylate functional group. In some embodiments, the linkers are located on the surface of the gold nanoparticles. In some embodiments, agents encompassed by the present invention can be modified to comprise the linkers. The linkers can comprise a PEG or oligoethylene glycol moiety with varying length to increase the particles' stability in biological environment and to control the density of the drug loads. PEG or oligoethylene glycol moieties also minimize nonspecific adsorption of undesired biomolecules. PEG or oligoethylene gycol moieties can be branched or linear (see, e.g., Tong et al. (2009) Langmuir 25:12454-12549). Agents encompassed by the present invention can be tethered to an amine-functionalized gold nanoparticles (see, e.g., Lippard et al. (2009) JACS 131:14652-14653). The cytotoxic effects for the Pt(IV)-gold nanoparticle complex are higher than the free Pt(IV) drugs and free cisplatin.

In some embodiments, agents encompassed by the present invention can be formulated with magnetic nanoparticles, such as those made from iron, cobalt, nickel, and oxides thereof, or iron hydroxide nanoparticles. Localized magnetic field gradients can be used to attract magnetic nanoparticles to a chosen site, to hold them until the therapy is complete, and then to remove them (see, e.g., Alexiou et al. (2000) Cancer Res. 60:6641-6648). In some embodiments, agents encompassed by the present invention can be bonded to magnetic nanoparticles with a linker. The linker can be a linker capable of undergoing an intramolecular cyclization to release agents. Any linker and nanoparticles disclosed can be used (see, e.g., PCT Publ. No. WO 2014/124329). Cyclization can be induced by heating the magnetic nanoparticle or by application of an alternating electromagnetic field to the magnetic nanoparticles.

In some embodiments, agents encompassed by the present invention are loaded onto iron oxide nanoparticles. In some embodiments, the agents encompassed by the present invention are formulated with super paramagnetic nanoparticles based on a core consisting of iron oxides (SPION). SPION are coated with inorganic materials (silica, gold, etc.) or organic materials (phospholipids, fatty acids, polysaccharides, peptides or other surfactants and polymers) and can be further functionalized with drugs, proteins or plasmids.

In one embodiment, water-dispersible oleic acid (OA)-poloxamer-coated iron oxide magnetic nanoparticles are used (see, e.g., Jain Mol. Pharm. (2005) 2:194-205) can be used to deliver the agents. Agents can partition into the OA shell surrounding the iron oxide nanoparticles and the poloxamer copolymers (e.g., Pluronics) confer aqueous dispersity to the formulation.

In some embodiments, nanoparticles having a phosphate moiety are used to deliver agents encompassed by the present invention (see, e.g., U.S. Pat. No. 8,828,975). The nanoparticles can comprise gold, iron oxide, titanium dioxide, zinc oxide, tin dioxide, copper, aluminum, cadmium selenide, silicon dioxide, and/or diamond. The nanoparticles can contain a PEG moiety on the surface.

In some embodiments, agents encompassed by the present invention can be formulated with peptides and/or other conjugates in order to increase penetration of cells such as macrophages and other immune cells. In one embodiment, peptides such as, but not limited to, cell penetrating peptides and proteins and peptides that enable intracellular delivery can be used to deliver pharmaceutical formulations. A non-limiting example of a cell-penetrating peptide that can be used with agents encompassed by the present invention include a cell-penetrating peptide sequence attached to polycations that facilitates delivery to the intracellular space, e.g., HIV-derived TAT peptide, penetratins, transportans, or hCT derived cell-penetrating peptides (see, e.g., Caron et al. (2001) Mol. Ther. 3:310-318; Langel, Cell-Penetrating Peptides: Processes and Applications (CRC Press, Boca Raton Fla., 2002); El-Andaloussi et al. (2003) Curr. Pharm. Des. 11:3597-35611; and Deshayes et al. (2005) Cell. Mol. Life Sci. 62:1839-1849).

In some embodiments, agents encompassed by the present invention can further comprise one or more conjugates that enhance delivery of the active agents (e.g., siRNA molecules) to targeted cells (e.g., monocytes, macrophages, and the like). The conjugate can be a ligand that can be incorporated into lipid formulations to specifically target cells of interest. Using a ligand targeting strategy for lipid particle drug delivery has the advantages of potentially increasing target specificity and avoiding the need for cationic lipids to trigger intracellular delivery. The ligand can include peptides, antibodies, proteins, polysaccharides, glycolipids, glycoproteins, and lectins which make use of mononuclear phagocytes characteristic receptor expression and phagocytic innate processes.

In some embodiments, the conjugated ligand can be a cell targeting peptide (CTP) or a cell-penetrating peptide (CPP) which can improve cell-specific targeting and cell uptake. A few example of the peptides include, but are not limited to muramyl tripeptide (MTP), RGD peptide, GGP-peptide that is selectively associated with monocytes (Karathanasis et al. (2009) Ann. Biomed. Engin. 37:1984-1992). The macrophage peptide targeting agent can also include those identified from phage display and sequencing (see, e.g., Liu et al. (2015) Bioconjug. Chem. 26:1811-1817). In some embodiments the ligand can be antibodies and fragments thereof, Exemplary antibodies specific to monocytes and macrophages include anti-VCAM-1 antibodies, anti-CC52 antibodies, anti-CC531 antibodies, anti-CD11c/DEC-205 antibodies. For example, antibodies can be coupled to the surface of liposomes or distally via their Fc-region to liposome-attached PEG.

In some embodiments, the nanoparticles can be mannosylated by incorporating into the lipid particles a lectin such as alkyl mannosides, Mann-C4-Chol, Mann-His-C4-Chol, Man2DOG, 4-aminophenyl-α-D-mannopyranoside, Aminophenyl-α-D-mannopyranoside, and Man3-DPPE. Immune cells, including alveolar macrophages, peritoneal macrophages, monocyte-derived dendritic cells, and Kupffer cells, constitutively express high levels of the mannose receptor (MR). Macrophages and DCs can therefore be targeted via mannosylated lipid nanoparticles.

Other ligands can also include maleylated bovine serum albumin (MBSA), O-steroly amylopectin (O-SAP), and fibronectin (see, e.g., Ahsan et al. (2002) J Cont. Rel. 79:29-40; Vyas et al. (2004) Intl. J. Pharm. 269:37-49).

### Other Components of Formulations

The compositions encompassed by the present invention can be incorporated into various formulations, including pharmaceutical formulations. The term “pharmaceutically acceptable” refers to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutical compositions encompassed by the present invention can be presented as anhydrous pharmaceutical formulations and dosage forms, liquid pharmaceutical formulations, solid pharmaceutical formulations, vaccines, and the like. Suitable liquid preparations can include, but are not limited to, isotonic aqueous solutions, suspensions, emulsions, or viscous compositions that are buffered to a selected pH.

As described in detail below, the agents and other compositions encompassed by the present invention can be specially formulated for administration in solid or liquid form, including those adapted for various routes of administration, such as (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound. Any appropriate form factor for an agent or composition described herein, such as, but not limited to, tablets, capsules, liquid syrups, soft gels, suppositories, and enemas, is contemplated.

Pharmaceutical compositions encompassed by the present invention can be presented as discrete dosage forms, such as capsules, sachets, or tablets, or liquids or aerosol sprays each containing a pre-determined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy. In some embodiments, the pharmaceutical compositions comprising the oligonucloetide compositions encompassed by the present invention can be formulated as, for example, solutions, emulsions (including microemulsions and creams), powders and liposome-containing formulations. The compositions can be formulated into any possible form factor such as, but not limited to, tablets, capsules, liquid syrups, soft gels, suppositories, and enemas.

In some embodiments, such formulations can also be constructed or compositions altered such that they passively or actively are directed to different cell types in vivo, including but not limited to monocytes, macrophages, and other immune cells (e.g., dendritic cells, antigen presenting cells, T lymphocytes, B lymphocytes, and natural killer cells), cancer cells and the like. Formulations can also be selectively targeted through expression of different ligands on their surface as exemplified by, but not limited by, folate, transferrin, N-acetylgalactosamine (GalNAc), and antibody targeted approaches.

The pharmaceutical compositions encompassed by the present invention can be formulated using one or more excipients to: (1) increase stability; (2) permit the sustained or delayed release (e.g., from a depot formulation); (3) alter the biodistribution (e.g., target an agent to a specific tissue or cell type); (4) alter the release profile of the agent in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients encompassed by the present invention can also include, without limitation, lipidoids, liposomes, lipid nanoparticles, polymers, lipoplexes, core-shell nanoparticles, peptides, proteins, hyaluronidase, nanoparticle mimics and combinations thereof.

The term “pharmaceutically acceptable carrier” or “pharmaceutically acceptable excipient” is intended to include any and all solvents, dispersion media, diluents or other liquid vehicles, dispersion or suspension agents, surface active agents, isotonic agents, thickening or emulsifying agents, disintegrating agents, preservatives, buffering agents, solid binders, lubricants, oils, coatings, antibacterial and antifungal agents, absorption delaying agents, and the like, as suited to the particular dosage form desired. Remington's The Science and Practice of Pharmacy, 21st Edition, A. R. Gennaro (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention. Supplementary active ingredients can also be incorporated into the described compositions.

In some embodiments, a pharmaceutically acceptable excipient is at least 95%, at least 96%, at least 97%, at least 98%, at least 99%, at least 99.5%, or at least 99.9% or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Various embodiments of the formulations can, optionally, include one or more of the following: buffer, pH adjuster, tonicity agent, cosolvent or pharmaceutically acceptable carrier.

In some embodiments, the formulation encompassed by the present invention can further comprise a buffer. A buffer is any substance that, when added to a solution, is capable of neutralizing both acids and bases without appreciably changing acidity or alkalinity of the solution. Examples of buffers include, but are not limited to, pharmaceutically acceptable salts and acids of acetate, glutamate, citrate, tartrate, benzoate, lactate, histidine, or other amino acids, gluconate, phosphate, malate, succinate, formate, propionate and carbonate.

In some embodiments, the formulation encompassed by the present invention can further comprise a pH adjuster. A pH adjuster is used to adjust the pH of the formulation. Suitable pH adjusters typically include at least an acid or a salt thereof and/or a base or a salt thereof. Acids and bases can be added on an as needed basis in order to achieve a desired pH. For example, if the pH is greater than the desired pH, an acid can be used to lower the pH to the desired pH. Examples of acids include, but are not limited to, hydrochloric acid, phosphoric acid, citric acid, ascorbic acid, acetic acid, sulfuric acid, carbonic acid and nitric acid. By way of another example, if the pH is less than the desired pH, a base can be used to adjust the pH to the desired pH. Examples of bases include, but are not limited to, sodium hydroxide, potassium hydroxide, calcium hydroxide, sodium carbonate, sodium citrate, sodium acetate and magnesium hydroxide.

In some embodiments, the formulation encompassed by the present invention can further comprise a tonicity agent. Tonicity agents are used to adjust the osmolality of the formulation in order to bring it closer to the osmotic pressure of body fluids, such as blood or plasma. Examples of tonicity agents include, but are not limited to, anhydrous or hydrous forms of sodium chloride, dextrose, sucrose, xylitol, fructose, glycerol, sorbitol, mannitol, potassium chloride, mannose, calcium chloride, magnesium chloride and other inorganic salts.

In some embodiments, the formulation encompassed by the present invention can further comprise a cosolvent. A cosolvent is a solvent that is added to the aqueous formulation in a weight amount that is less than that of water and assists in the solubilization of the aptamer. Examples of cosolvents include, but are not limited to, glycols, ethanol and polyhydric alcohols.

In some embodiments, the formulation encompassed by the present invention can further comprise a “pharmaceutically acceptable excipient.” As used herein, the term “pharmaceutically acceptable carrier” or “excipient” is a pharmaceutically acceptable inactive substance formulated alongside with the active ingredient of a medication (e.g., siRNA molecules of CCR2 and/or CSF1R).

The excipient can be liquid or solid and is selected, with the planned manner of administration in mind, so as to provide for the desired bulk, consistency, etc., when combined with a nucleic acid and the other components of a given pharmaceutical composition. The pharmaceutically acceptable excipients can be used for different purposes, for example, as anti-adherents that reduce the adhesion between the powder (e.g., pregelatinized maize starch, polyvinylpyrrolidone or hydroxypropyl methylcellulose, etc.), binders that hold the ingredients in a tablet together (e.g., saccharides and their derivatives, gelatin, synthetic polymers: polyvinylpyrrolidone (PVP), polyethylene glycol (PEG)), coatings (e.g., cellulose ether hydroxypropyl methylcellulose (HPMC) film coating for tablets, polymers, shellac, corn protein zein, polysaccharides, etc.), disintegrants (e.g., crosslinked polymers, crosslinked polyvinylpyrrolidone (crospovidone)), crosslinked sodium carboxymethyl cellulose (croscarmellose sodium), modified starch sodium starch glycolate), fillers (e.g., lactose and other sugars, microcrystalline cellulose, pectin, gelatin, calcium sulfate, ethyl cellulose, polyacrylates or calcium hydrogen phosphate, etc.), flavors (e.g., natural fruit extracts, etc.), colors (e.g., to improve the appearance of a formulation), lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), glidants (e.g., fumed silica, talc, and magnesium carbonate, etc.), sorbents, preservatives (e.g., antioxidants amino acids cysteine, methionine, citric acid, methyl paraben, etc.), sweeteners (e.g., sugar), and wetting agents (e.g., sodium lauryl sulphate, etc.). Pharmaceutically acceptable carriers are well known in the art. Examples of pharmaceutically acceptable carriers can be found, for example, in Goodman and Gillman, The Pharmacological Basis of Therapeutics, latest edition.

The formulations encompassed by the present invention can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components (e.g., siRNA molecules of CCR2 and/or CSF1R) of the compositions encompassed by the present invention.

In some embodiments, the composition encompassed by the present invention can also be formulated as suspensions in aqueous, non-aqueous, or mixed media. Aqueous suspensions can further contain substances which increase the viscosity of the suspension including, for example, sodium carboxymethylcellulose, sorbitol and/or dextran. The suspension can also contain stabilizers.

In some embodiments, the pharmaceutical compositions encompassed by the present invention can be formulated and used as foams. Pharmaceutical foams include formulations such as, but not limited to, emulsions, microemulsions, creams, jellies and formulations containing liposomes.

The pharmaceutical compositions encompassed by the present invention can be formulated as emulsions. Emulsions are typically heterogeneous systems of one liquid dispersed in another in the form of droplets usually exceeding 0.1 um in diameter. (See, e.g. Idson, in Pharmaceutical Dosage Forms. Disperse Systems, Vol. 1). Emulsions are often biphasic systems comprising of two immiscible liquid phases intimately mixed and dispersed with each other. In general, emulsions can be either water in oil (w/o) or of the oil in water (o/w) variety. Emulsions can contain additional components in addition to the dispersed phases and the active components (e.g., siRNA molecules specific to CCR2 and/or CSF1R) which can be present as a solution in the aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases, such as, for example, in the case of oil in water in oil (o/w/o) and water in oil in water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise, a system of oil droplets enclosed in globules of water stabilized in an oily continuous provides an o/w/o emulsion.

In some embodiments, the pharmaceutical compositions encompassed by the present invention are formulated as microemulsions. A microemulsion can be defined as a system of water, oil and amphiphile which is a single optically isotropic and thermodynamically stable liquid solution (see, e.g., Rosoff, in Pharmaceutical Dosage Forms: Disperse Systems, Vol. 1). Microemulsions commonly are prepared via a combination of three to five components that include oil, water, surfactant, cosurfactant and electrolyte.

In some embodiments, the pharmaceutical compositions encompassed by the present invention are reconstituted with a suitable diluent, e.g., sterile water or sterile saline for subcutaneous or intravenous injection.

### V. Dosage and Route of Administration

### Dosing

The pharmaceutical compositions in accordance with the invention are typically formulated in dosage unit form for ease of administration and uniformity of dosage. It will be understood, however, that the total daily usage of the compositions encompassed by the present invention can be decided by the attending physician within the scope of sound medical judgment. The specific therapeutically effective, prophylactically effective, or appropriate preventing dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known.

The total dosage can be administered in a single dose, multiple doses, repeated doses, as a continual dose or a combination thereof. In some embodiments, pharmaceutical compositions encompassed by the present invention can be administered in a single daily dose, or the total daily dosage can be administered in divided doses of two, three or four times daily.

The formulations and dosages described herein are designed to maximize clinical efficacy in the treatment of diseases and disorders while simultaneously decreasing or minimizing adverse side effects.

In some embodiments, agents in accordance with the present invention can be administered at dosage levels sufficient to deliver from about 0.0001 mg/kg to about 1000 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, or from about 10 mg/kg to about 100 mg/kg, or from about 100 mg/kg to about 500 mg/kg, of subject body weight per day, one or more times a day, to obtain the desired therapeutic, diagnostic, prophylactic, or imaging effect. The desired dosage can be delivered three times a day, two times a day, once a day, every other day, every third day, every week, every two weeks, every three weeks, or every four weeks, or every two months. In some embodiments, the desired dosage can be delivered using multiple administrations (e.g., two, three, four, five, six, seven, eight, nine, ten, eleven, twelve, thirteen, fourteen, or more administrations). When multiple administrations are employed, split dosing regimens such as those described herein can be used.

In some embodiments, an agent encompassed by the present invention is an antibody. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors can influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment can increase or decrease over the course of a particular treatment. Changes in dosage can result from the results of diagnostic assays.

As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hour period. It can be administered as a single unit dose.

Cells can be administered at 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, or more, or any range in between or any value in between, cells per kilogram of subject body weight. The number of cells transplanted can be adjusted based on the desired level of engraftment in a given amount of time. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, can be transplanted. In some embodiment, transplantation of at least about 0.1×10⁶, 0.5×10⁶, 1.0×10⁶, 2.0×10⁶, 3.0×10⁶, 4.0×10⁶, or 5.0×10⁶ total cells relative to an average size mouse is effective.

Cells can be administered in any suitable route as described herein, such as by infusion. Cells can also be administered before, concurrently with, or after, other anti-cancer agents.

### Routes of Administration

Administration can be accomplished using methods generally known in the art. Agents, including cells, can be introduced to the desired site by direct injection, or by any other means used in the art including, but are not limited to, intra-tumoral, intravascular, intracerebral, parenteral, intraperitoneal, intravenous, epidural, intraspinal, intrasternal, intra-articular, intra-synovial, intrathecal, intra-arterial, intracardiac, or intramuscular administration.

For example, compositions and formulations are usually administered through either parenteral or non-parenteral routes to a subject. Parenteral administration relates to a pharmaceutical composition administered to a body in a manner other than through the digestive tract, such as by intravenous or intramuscular injection. Parenteral administration can include administration intraarticularly, intravenously, intraperitoneally, subcutaneously, and intramuscularly.

In some embodiments, non-parenteral administration can be used including, but not limited to, buccal, sublingual, endoscopic, oral, rectal, transdermal, topical, nasal, intratracheal, pulmonary, urethral, vaginal, and ocular. When administered by such non-parenteral modes, the methods and pharmaceutical composition encompassed by the present invention can deliver the drug both locally and systemically as desired.

Cell-based agents can be administered in one infusion, or through successive infusions over a defined time period sufficient to generate a desired effect. Exemplary methods for transplantation, engraftment assessment, and marker phenotyping analysis of transplanted cells are well-known in the art (see, for example, Pearson et al. (2008) Curr. Protoc. Immunol. 81:15.21.1-15.21.21; Ito et al. (2002) Blood 100:3175-3182; Traggiai et al. (2004) Science 304:104-107; Ishikawa et al. Blood (2005) 106:1565-1573; Shultz et al. (2005) J. Immunol. 174:6477-6489; and Holyoake et al. (1999) Exp. Hematol. 27:1418-1427).

Two or more cell types can be combined and administered, such as cell-based therapy and adoptive cell transfer of stem cells, cancer vaccines and cell-based therapy, and the like. For example, adoptive cell-based immunotherapies can be combined with the cell-based therapies of the present invention. In some embodiments, the cell-based agents can be used alone or in combination with additional cell-based agents, such as immunotherapies like adoptive T cell therapy (ACT). For example, T cells genetically engineered to recognize CD19 used to treat follicular B cell lymphoma. Immune cells for ACT can be dendritic cells, T cells such as CD8⁺ T cells and CD4⁺ T cells, natural killer (NK) cells, NK T cells, cytotoxic T lymphocytes (CTLs), tumor infiltrating lymphocytes (TILs), lymphokine activated killer (LAK) cells, memory T cells, regulatory T cells (Tregs), helper T cells, cytokine-induced killer (CIK) cells, and any combination thereof. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like. The ratio of an agent encompassed by the present invention, such as cancer cells, to another agent encompassed by the present invention or other composition can be 1:1 relative to each other (e.g., equal amounts of 2 agents, 3 agents, 4 agents, etc.), but can modulated in any amount desired (e.g., 1:1, 1.1:1, 1.2:1, 1.3:1, 1.4:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, or greater).

Engraftment of transplanted cells can be assessed by any of various methods, such as, but not limited to, tumor volume, cytokine levels, time of administration, flow cytometric analysis of cells of interest obtained from the subject at one or more time points following transplantation, and the like. For example, a time-based analysis of waiting 1, 2, 3, 4, 5, 6, 7, 8, 9, 10, 11, 12, 13, 14, 15, 16, 17, 18, 19, 20, 21, 22, 23, 24, 25, 26, 27, 28 days or can signal the time for tumor harvesting. Any such metrics are variables that can be adjusted according to well-known parameters in order to determine the effect of the variable on a response to anti-cancer immunotherapy. In addition, the transplanted cells can be co-transplanted with other agents, such as cytokines, extracellular matrices, cell culture supports, and the like.

The pharmaceutical compositions comprising the siRNA molecules of CCR2 and CSF1R are administered to subjects in need, preferably human subjects, in an amount effective to modulate the activity of myeloid-derived cells, such as monocytes and/or macrophages, associated with diseases, such as cancers.

### VI. Uses and Methods

The present invention provides methods of inhibiting the activity of CCR2 and CSF1R receptors comprising contacting a myeloid-derived cell (e.g., a monocyte and/or macrophage) with an effective amount of an oligonucleotide composition targeting CCR2, an oligonucleotide composition targeting CSF1R, an oligonucleotide targeting both CCR2 and CSF1R, and/or an oligonucleotide composition targeting CCR2 in combination with an oligonucleotide composition targeting CSF1R, encompassed by the present invention, wherein the siRNA molecule cocktail is sufficient to inhibit the expression of CCR2 and/or CSF1R in the cell. In some embodiments, the oligonucleotide composition can further comprise at least one additional therapeutic agent, such one or more antagonists of CCL2 and CSF1, immunotherapeutic agent, and the like.

The compositions, agents, and formulations described herein can be used in a variety of modulatory, therapeutic, screening, diagnostic, prognostic, and therapeutic applications described herein, such as a modulatory method, therapeutic method, screening method, diagnostic method, prognostic method, or combinations thereof. All steps of any such method or methods can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

In addition, any aspect of the present invention described herein can be performed either alone or in combination with any other aspect of the present invention, including one, more than one, or all embodiments thereof. For example, diagnostic and/or screening methods can be performed alone or in combination with a treatment step, such as providing an appropriate therapy upon determining an appropriate diagnosis and/or screening result.

One aspect encompassed by the present invention relates to methods of modulating the copy number, amount (e.g., expression), and/or activity (e.g., modulating subcellular localization) of at least one biomarker (e.g., one or more targets listed in Table 1, Table 2, the Examples, etc.) described herein, such as for therapeutic purposes. Such agents can be used to manipulate myeloid-derived cells. In one embodiment, a particular subpopulation of monocytes and/or macrophages is manipulated to regulate their numbers and/or activities in a physiological condition. For example, compositions encompassed by the present invention can modulate the expression of CCR2 and/or CSF1R to thereby modulate the inflammatory phenotype of myeloid-derived cells, including monocytes and macrophages, and further modulate immune responses. In some embodiments, cell activities (e.g., cytokine secretion, cell population ratios, etc.) are modulated rather than modulating immune responses per se. Methods for modulating myeloid-cell derived cell inflammatory phenotypes using the compositions and formulations disclosed herein, are provided. Accordingly, the compositions and methods can be used for modulating immune responses by modulating CCR2 and/or CSF1R expression, which depletes or enriches certain types of cells and/or to modulate the ratio of cell types. For example, inhibiting CCR2 and/or CSF1R expression on such cells increases pro-inflammatory monocytes/macrophages versus anti-inflammatory monocytes/macrophages. In some embodiments, the compositions are used to treat cancer in a subject afflicted with a cancer.

The present disclosure demonstrates that the downregulation of the expression of CCR2 and/or CSF1R in myeloid-derived cells, including monocytes and macrophages can re-polarize (e.g., change the inflammatory phenotype) of the cells. In some embodiments, the phenotype of an M2 macrophage is changed to result in a macrophage with a Type 1 or M1 phenotype, or vice versa regarding M1 macrophages and Type 2 or M2 phenotypes. In some embodiments, compositions encompassed by the present invention are used to inhibit the trafficking, polarization, and/or activation of monocytes and macrophages with an M2 phenotype, or vice versa regarding Type 1 and M1 macrophages. The present invention further provides methods for reducing populations of monocytes and/or macrophages of interest, such as M1 macrophages, M2 macrophages (e.g., TAMs in a tumor), and the like.

In some embodiments, the present invention provides methods for changing the distribution of monocytes and/or macrophages, including subtypes thereof, such as pro-tumoral macrophages and anti-tumoral macrophages. In one example, the present invention provides methods for driving macrophages towards a pro-inflammatory immune response from an anti-inflammatory immune response and vice versa. Cell types can be depleted and/or enriched by 5%, 10%, 15%, 20%, 25%, 30%, 35%, 40%, 45%, 50%, 55%, 60%, 65%, 70%, 75%, 80%, 85%, 90%, 95%, 99%, or more, or any range in between inclusive, such as 45-55%.

In some embodiments, the modulation occurs in cells, such as monocyte, macrophage, or other phagocyte, like a dendritic cell. In some embodiments, the cell is a macrophage subtype, such as a macrophage subtype described herein. For example, the macrophage can be a tissue resident macrophage (TAM) or a macrophage derived from a circulating monocyte in the bloodstream.

In some embodiments, modulating monocyte and/or macrophage inflammatory phenotypes results in desired modulated immune responses, such as modulation of abnormal monocyte migration and proliferation, unregulated proliferation of tissue resident macrophages, unregulated pro-inflammatory macrophages, unregulated anti-inflammatory macrophages, unbalanced distribution of pro-inflammatory and anti-inflammatory macrophage subpopulations in a tissue, an abnormally adopted activation state of monocytes and macrophages in a disease condition, modulated cytotoxic T-cell activation and function, overcoming of resistance of cancer cells to therapy, and sensitivity of cancer cells to immunotherapy, such as immune checkpoint therapy.

Methods for treating and/or preventing a disease associated with unwanted myeloid-derived cell phenotypes comprise contacting such cells, either in vitro, ex vivo, or in vivo (e.g., administering to a subject), with compositions encompassed by the present invention, wherein the compositions manipulate the migration, recruitment, differentiation and polarization, activation, function, and/or survival of the cells.

In one aspect encompassed by the present invention, methods for increasing pro-inflammatory activities of monocytes and/or macrophages are provided.

In another aspect encompassed by the present invention, methods for balancing pro-inflammatory monocytes and macrophages and anti-inflammatory monocytes and macrophages in a tissue are provided.

### Subjects

The present invention provides methods of treating an individual afflicted with a condition or disorder that would benefit from inhibition of CCR2 and/or CSF1R, e.g., a disorder characterized by unwanted CCR2 and/or CSF1R expression or activity comprising contacting myeloid-derived cells of interest with at least one composition encompassed by the present invention.

In some embodiments, the subject is an animal. The animal can be of either sex and can be at any stage of development. In some embodiments, the animals is a vertebrate, such as a mammal. In some embodiments, the subject is a non-human mammal. In some embodiments, the subject is a domesticated animal, such as a dog, cat, cow, pig, horse, sheep, or goat. In some embodiments, the subject is a companion animal, such as a dog or cat. In some embodiments, the subject is a livestock animal, such as a cow, pig, horse, sheep, or goat. In some embodiments, the subject is a zoo animal. In some embodiments, the subject is a research animal, such as a rodent (e.g., mouse or rat), dog, pig, or non-human primate. In some embodiments, the animal is a genetically engineered animal. In some embodiments, the animal is a transgenic animal (e.g., transgenic mice and transgenic pigs). In some embodiments, the subject is a fish or reptile. In some embodiments, the subject is a human. In some embodiments, the subject is an animal model of cancer. For example, the animal model can be an orthotopic xenograft animal model of a human-derived cancer.

In some embodiments of the methods encompassed by the present invention, the subject has not undergone treatment, such as chemotherapy, radiation therapy, targeted therapy, and/or immunotherapies. In some embodiments, the subject has undergone treatment, such as chemotherapy, radiation therapy, targeted therapy, and/or immunotherapies.

In some embodiments, the subject has had surgery to remove cancerous or precancerous tissue. In some embodiments, the cancerous tissue has not been removed, e.g., the cancerous tissue can be located in an inoperable region of the body, such as in a tissue that is essential for life, or in a region where a surgical procedure would cause considerable risk of harm to the patient.

In some embodiments, the subject or cells thereof are resistant to a therapy of relevance, such as resistant to immune checkpoint inhibitor therapy. For example, modulating one or more biomarkers encompassed by the present invention can overcome resistance to immune checkpoint inhibitor therapy.

In some embodiments, the subjects are in need of modulation according to compositions and methods described herein, such as having been identified as having an unwanted absence, presence, or aberrant expression and/or activity of one or more biomarkers described herein.

In addition, these modulatory agents can also be administered in combination therapy to further modulate a desired activity, such as stimulating immune responses. For examples, agents and compositions that target to IL-4, IL-4Ra, IL-13, and CD40 can be used to modulate monocyte and/or macrophage differentiation and/or polarization. Agents and compositions that target to CD11b, CSF-1R, CCL2, neurophilim-1 and ANG-2 can be used to modulate macrophage recruitment to a tissue. Agents and compositions that target to IL-6, IL-6R and TNF-α can be used to modulate macrophage function. Additional agents include, without limitations, chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, these modulatory agents can be administered with a therapeutically effective dose of chemotherapeutic agent. In another embodiment, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular melanoma, being treated, the extent of the disease and other factors familiar to the physician of skill in the art and can be determined by the physician.

### Cancer Therapies

In some embodiments, compositions encompassed by the present invention are used to treat cancer. For example, the present invention provides methods for reducing pro-tumoral functions of myeloid-derived cells including monocytes and macrophages (i.e., tumorigenicity) and/or increasing anti-tumoral functions of myeloid-derived cells including monocytes and macrophages. In some particular embodiments, the method encompassed by the present invention can reduce at least one of the pro-tumoral functions of macrophages including 1) recruitment and polarization of tumor associate macrophages (TAMs), 2) tumor angiogenesis, 3) tumor growth, 4) tumor cell differentiation, 5) tumor cell survival, 6) tumor invasion and metastasis, 7) immune inhibition, and 8) immunosuppressive tumor microenvironment.

Cancer therapy or combinations of therapies including the use of compositions encompassed by the present invention can be used to contact cancer cells and/or administered to a desired subject, such as a subject that is indicated as being a likely responder to cancer therapy. In another embodiment, such cancer therapy can be avoided once a subject is indicated as not being a likely responder to the cancer therapy (e.g., a subject whose myeloid-derived cells do not express appreciable or desired levels of CCR2 and/or CSF1R) and an alternative treatment regimen, such as targeted and/or untargeted cancer therapies can be administered. Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with or without cancer therapy (e.g., at least one modulator of one or more targets listed in Table 1 and/or Table 2).

Representative exemplary compositions useful for inhibiting CCR2 and/or CSF1R are described above. As described further below, anti-cancer agents encompass biotherapeutic anti-cancer agents (e.g., interferons, cytokines (e.g., tumor necrosis factor, interferon α, interferon γ, etc.), vaccines, hematopoietic growth factors, monoclonal serotherapy, immunostimulants and/or immunodulatory agents (e.g., IL-1, 2, 4, 6, and/or 12), immune cell growth factors (e.g., GM-CSF), and antibodies (e.g., trastuzumab, T-DM1, bevacizumab, cetuximab, panitumumab, rituximab, tositumomab, and the like), as well as chemotherapeutic agents.

The term “targeted therapy” refers to administration of agents that selectively interact with a chosen biomolecule to thereby treat cancer. For example, targeted therapy regarding the inhibition of immune checkpoint inhibitor is useful in combination with the methods encompassed by the present invention.

The term “immunotherapy” or “immunotherapies” generally refers to any strategy for modulating an immune response in a beneficial manner and encompasses the treatment of a subject afflicted with, or at risk of contracting or suffering a recurrence of, a disease by a method comprising inducing, enhancing, suppressing or otherwise modifying an immune response, as well as any treatment that uses certain parts of a subject's immune system to fight diseases, such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” In some embodiments, an immunotherapy is specific for cells of interest, such as cancer cells. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Some forms of immunotherapy are targeted therapies that can comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They can also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer. Similarly, immunotherapy can take the form of cell-based therapies. For example, adoptive cellular immunotherapy is a type of immunotherapy using immune cells, such as T cells, that have a natural or genetically engineered reactivity to a patient's cancer are generated and then transferred back into the cancer patient. The injection of a large number of activated tumor-specific T cells can induce complete and durable regression of cancers.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In some embodiments, an immunotherapeutic agent is an agonist of an immune-stimulatory molecule; an antagonist of an immune-inhibitory molecule; an antagonist of a chemokine; an agonist of a cytokine that stimulates T cell activation; an agent that antagonizes or inhibits a cytokine that inhibits T cell activation; and/or an agent that binds to a membrane bound protein of the B7 family. In some embodiments, the immunotherapeutic agent is an antagonist of an immune-inhibitory molecule. In some embodiments, the immunotherapeutic agents can be agents for cytokines, chemokines and growth factors, for examples, neutralizing antibodies that neutralize the inhibitory effect of tumor associated cytokines, chemokines, growth factors and other soluble factors including IL-10, TGF-β and VEGF.

In some embodiments, immunotherapy comprises inhibitors of one or more immune checkpoints. The term “immune checkpoint” refers to a group of molecules on the cell surface of CD4+ and/or CD8+ T cells that fine-tune immune responses by modulating anti-cancer immune responses, such as down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD200R, CD160, gp49B, PIR-B, KRLG-1, KIR family receptors, TIM-1, TIM-3, TIM-4, LAG-3 (CD223), IDO, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR (see, for example, WO 2012/177624).

Some immune checkpoints are “immune-inhibitory immune checkpoints” encompassing molecules (e.g., proteins) that inhibit, down-regulate, or suppress a function of the immune system (e.g., an immune response). For example, PD-L1 (programmed death-ligand 1), also known as CD274 or B7-H1, is a protein that transmits an inhibitory signal that reduces proliferation of T cells to suppress the immune system. CTLA-4 (cytotoxic T-lymphocyte-associated protein 4), also known as CD152, is a protein receptor on the surface of antigen-presenting cells that serves as an immune checkpoint (“off” switch) to downregulate immune responses. TIM-3 (T-cell immunoglobulin and mucin-domain containing-3), also known as HAVCR2, is a cell surface protein that serves as an immune checkpoint to regulate macrophage activation. VISTA (V-domain Ig suppressor of T cell activation) is a type I transmembrane protein that functions as an immune checkpoint to inhibit T cell effector function and maintain peripheral tolerance. LAG-3 (lymphocyte-activation gene 3) is an immune checkpoint receptor that negatively regulates proliferation, activation, and homeostasis of T cells. BTLA (B- and T-lymphocyte attenuator) is a protein that displays T cell inhibition via interactions with tumor necrosis family receptors (TNF-R). KIR (killer-cell immunoglobulin-like receptor) is a family of proteins expressed on NK cells, and a minority of T cells, that suppress the cytotoxic activity of NK cells. In some embodiments, immunotherapeutic agents can be agents specific to immunosuppressive enzymes such as inhibitors that can block the activities of arginase (ARG) and indoleamine 2,3-dioxygenase (IDO), an immune checkpoint protein that suppresses T cells and NK cells, which change the catabolism of the amino acids arginine and tryptophan in the immunosuppressive tumor microenvironment. The inhibitors can include, but are not limited to, N-hydroxy-L-Arg (NOHA) targeting to ARG-expressing M2 macrophages, nitroaspirin or sildenafil (Viagra®), which blocks ARG and nitric oxide synthase (NOS) simultaneously; and IDO inhibitors, such as 1-methyl-tryptophan. The term further encompasses biologically active protein fragment, as well as nucleic acids encoding full-length immune checkpoint proteins and biologically active protein fragments thereof. In some embodiment, the term further encompasses any fragment according to homology descriptions provided herein.

By contrast, other immune checkpoints are “immune-stimulatory” encompassing molecules (e.g., proteins) that activate, stimulate, or promote a function of the immune system (e.g., an immune response). In some embodiments, the immune-stimulatory molecule is CD28, CD80 (B7.1), CD86 (B7.2), 4-1BB (CD137), 4-1BBL (CD137L), CD27, CD70, CD40, CD40L, CD122, CD226, CD30, CD30L, OX40, OX40L, HVEM, BTLA, GITR and its ligand GITRL, LIGHT, LTβR, LTαβ, ICOS (CD278), ICOSL (B7-H2), and NKG2D. CD40 (cluster of differentiation 40) is a costimulatory protein found on antigen presenting cells that is required for their activation. OX40, also known as tumor necrosis factor receptor superfamily member 4 (TNFRSF4) or CD134, is involved in maintenance of an immune response after activation by preventing T-cell death and subsequently increasing cytokine production. CD137 is a member of the tumor necrosis factor receptor (TNF-R) family that co-stimulates activated T cells to enhance proliferation and T cell survival. CD122 is a subunit of the interleukin-2 receptor (IL-2) protein, which promotes differentiation of immature T cells into regulatory, effector, or memory T cells. CD27 is a member of the tumor necrosis factor receptor superfamily and serves as a co-stimulatory immune checkpoint molecule. CD28 (cluster of differentiation 28) is a protein expressed on T cells that provides co-stimulatory signals required for T cell activation and survival. GITR (glucocorticoid-induced TNFR-related protein), also known as TNFRSF18 and AITR, is a protein that plays a key role in dominant immunological self-tolerance maintained by regulatory T cells. ICOS (inducible T-cell co-stimulator), also known as CD278, is a CD28-superfamily costimulatory molecule that is expressed on activated T cells and play a role in T cell signaling and immune responses.

Immune checkpoints and their sequences are well-known in the art and representative embodiments are described further below. Immune checkpoints generally relate to pairs of inhibitory receptors and the natural binding partners (e.g., ligands). For example, PD-1 polypeptides are inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells. The term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell inhibitory signals, and the ability to modulate the immune response. The term “PD-1 ligand” refers to binding partners of the PD-1 receptor and includes both PD-L1 (Freeman et al. (2000) J Exp. Med. 192:1027-1034) and PD-L2 (Latchman et al. (2001) Nat. Immunol. 2:261). The term “PD-1 ligand activity” includes the ability of a PD-1 ligand polypeptide to bind its natural receptor(s) (e.g., PD-1 or B7-1), the ability to modulate immune cell inhibitory signals, and the ability to modulate the immune response.

As used herein, the term “immune checkpoint therapy” refers to the use of agents that inhibit immune-inhibitory immune checkpoints, such as inhibiting their nucleic acids and/or proteins. Inhibition of one or more such immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins that block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g., the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. Therapeutic agents used for blocking the PD-1 pathway include antagonistic antibodies and soluble PD-L1 ligands. The antagonist agents against PD-1 and PD-L1/2 inhibitory pathway can include, but are not limited to, antagonistic antibodies to PD-1 or PD-L1/2 (e.g., 17D8, 2D3, 4H1, 5C4 (also known as nivolumab or BMS-936558), 4A11, 7D3 and 5F4 disclosed in U.S. Pat. No. 8,008,449; AMP-224, pidilizumab (CT-011), pembrolizumab, and antibodies disclosed in U.S. Pat. Nos. 8,779,105; 8,552,154; 8,217,149; 8,168,757; 8,008,449; 7,488,802; 7,943,743; 7,635,757; and 6,808,710. Similarly, additional representative checkpoint inhibitors can be, but are not limited to, antibodies against inhibitory regulator CTLA-4 (anti-cytotoxic T-lymphocyte antigen 4 anti-cytotoxic T-lymphocyte antigen 4), such as ipilimumab, tremelimumab (fully humanized), anti-CD28 antibodies, anti-CTLA-4 adnectins, anti-CTLA-4 domain antibodies, single chain anti-CTLA-4 antibody fragments, heavy chain anti-CTLA-4 fragments, light chain anti-CTLA-4 fragments, and other antibodies, such as those disclosed in U.S. Pat. Nos. 8,748,815; 8,529,902; 8,318,916; 8,017,114; 7,744,875; 7,605,238; 7,465,446; 7,109,003; 7,132,281; 6,984,720; 6,682,736; 6,207,156; and 5,977,318, as well as EP Pat. No. 1212422, U.S. Pat Publ. Numbers 2002/0039581 and 2002/086014, and Hurwitz et al. (1998) Proc. Natl. Acad. Sci. U.S.A. 95:10067-10071.

The representative definitions of immune checkpoint activity, ligand, blockade, and the like exemplified for PD-1, PD-L1, PD-L2, and CTLA-4 apply generally to other immune checkpoints.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent can be, but is not limited to, those selected from among the following groups of compounds: platinum compounds, cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary agents include, but are not limited to, alkylating agents: nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Similarly, additional exemplary agents including platinum-ontaining compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genentech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. Compositions comprising one or more chemotherapeutic agents (e.g., FLAG, CHOP) can also be used. FLAG comprises fludarabine, cytosine arabinoside (Ara-C) and G-CSF. CHOP comprises cyclophosphamide, vincristine, doxorubicin, and prednisone. In another embodiment, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of beta-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard et. al. (2003) Exp. Hematol. 31:446-454); Herceg (2001) Mut. Res. 477:97-110). Poly(ADP-ribose) polymerase 1 (PARP1) is a key molecule in the repair of DNA single-strand breaks (SSBs) (de Murcia J. et al. (1997) Proc. Natl. Acad. Sci. U.S.A. 94:7303-7307; Schreiber et al. (2006) Nat. Rev. Mol. Cell Biol. 7:517-528; Wang et al. (1997) Genes Dev. 11:2347-2358). Knockout of SSB repair by inhibition of PARP1 function induces DNA double-strand breaks (DSBs) that can trigger synthetic lethality in cancer cells with defective homology-directed DSB repair (Bryant et al. (2005) Nature 434:913-917; Farmer et al. (2005) Nature 434:917-921). The foregoing examples of chemotherapeutic agents are illustrative and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In another embodiment, hyperthermia, a procedure in which body tissue is exposed to high temperatures (up to 106° F.) is used. Heat can help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area can be heated externally with high-frequency waves aimed at a tumor from a device outside the body. To achieve internal heating, one of several types of sterile probes can be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early nonsmall cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It can also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and can be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) lasers can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time can be needed because the precision of the laser allows for a smaller incision. Healing time is often shortened; since laser heat seals blood vessels, there is less bleeding, swelling, or scarring. Laser surgery can be less complicated. For example, with fiber optics, laser light can be directed to parts of the body without making a large incision. More procedures can be done on an outpatient basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They can be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also can be used with low-power microscopes, giving the doctor a clear view of the site being treated. Used with other instruments, laser systems can produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers can be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat can help shrink tumors by damaging cells or depriving them of substances they need to live. In this treatment, lasers are directed to interstitial areas (areas between organs) in the body. The laser light then raises the temperature of the tumor, which damages or destroys cancer cells.

### VII. Kits

The present invention also encompasses kits comprising the compositions and formulations encompassed by the present invention. A “kit” is any manufacture (e.g. a package or container) comprising at least one reagent, e.g. an oligonucleotide composition, for specifically detecting and/or affecting the expression of CCR2 and/or CSF1R. The kit can be promoted, distributed, or sold as a unit for performing the methods of the present invention. The kit can comprise one or more reagents necessary to detect, inhibit, screen, etc. that are useful in the methods of the present invention.

Reagents in the kit can be provided in individual containers or as mixtures of two or more reagents in a single container. In addition, instructional materials which describe the use of the compositions within the kit can be included. A kit encompassed by the present invention can also include instructional materials disclosing or describing the use of the kit for a method encompassed by the present invention as provided herein. A kit can also include additional components to facilitate the particular application for which the kit is designed. For example, a kit can additionally contain controls (e.g., control biological samples or standards). A kit can additionally include buffers and other reagents recognized for use in a method of the disclosed invention.

Other embodiments encompassed by the present invention are described in the following Examples. The present invention is further illustrated by the following examples which should not be construed as further limiting.

### EXAMPLES

### Example 1: Identification of Candidate siRNAs Targeting the Human CCR2 mRNA

Design of siRNAs Using Bioinformatics Algorithms

Human CCR2 mRNA sequence (Gene Bank NO. NM_001123041.2; SEQ ID NO: 1) was used as the target template. All possible 19-mer siRNA molecules were created from this reference sequence. At the same time, the off-target genes of all possible siRNAs were predicted for human, non-human primates (NHPs), such as rhesus monkey and cynomolgus monkey, and mouse and rat, as well. A specificity score was assigned to each siRNA strand analyzed and compared. More than 900 siRNA candidates directed against human CCR2 transcripts were created and further evaluated.

Evaluation of siRNA Candidates

For all the siRNA candidates, target specificity, intra- and inter-species cross-activity, activity and other key features were evaluated.

### Target Specificity

The siRNA candidates with lowest sequence complementarity to any non-target transcript and siRNA candidates whose seed regions (around positions 2-7) is ideally not identical to a seed region (positions 2-7) of known microRNA molecules are identified.

For each predicted siRNA candidate, off-gene targets were predicted for human, rhesus monkey and cynomolgus monkey. A specificity score was assigned to each siRNA strand (i.e., sense strand and antisense strand). Each siRNA strand with a specificity score was categorized and analyzed. The specificity score considers the likelihood of unintended downregulation of any other transcript by full or partial complementarity of a siRNA strand (up to 4 mismatches within positions 2-18 of 19-mer) and the score describes the predicted most likely off-target(s) for antisense and sense strand of each siRNA molecule by transcriptome-wide off-target analysis. The off-target frequency was categorized by numbers of mismatches (e.g., from 0 mismatch to 4 mismatches). Another criteria then was analyzed for target specificity. siRNAs can function in a miRNA-like manner via base-pairing with complementary sequences within the 3′-UTR of mRNA molecules. That complementarity typically encompasses the 5′-end 2-7 of the miRNA (seed region). In order to circumvent siRNAs to act via functional miRNA binding sites, siRNA strands that contain natural miRNA seed regions were evaluated and avoided. Furthermore, conserved seed regions in miRNAs from human, mouse, rat, rhesus monkey, dog and pig were also examined (data received from the miRBase database).

TABLE 6
Target specificity classification criteria

| | | | Contain | Contain |
|---|---|---|---|---|
| | | Specificity | miRNA | conserved |
| Category | | score | seed | miRNA seed |
| 1 | highly specific | ≥3 | NO | NO |
| 2 | specific | ≥2 | YES/NO | NO |
| 3 | minimal specificity | ≥1 | YES/NO | NO |
| 4 | (A) Specificity not | — | YES/NO | YES/NO |
| | considered | | | |
| | (B) unspecific | 0 | YES/NO | YES/NO |
| | (C) unspecific | — | YES/NO | YES |

Based on these criteria (as shown in Table 6), highly specific siRNA molecules were selected for further evaluation.

### Cross-Species Reactivity

siRNA candidates that target at least all-protein coding transcripts of the target gene CCR2 and for each species were selected. Sequences including transcript variants from different species were analyzed for cross-reactivity (Table 7). The analysis was separately performed for all 19-mers and 17-mers (positions of 2-18 of 19-mer) with full match to the target sequences in primary species, and for siRNAs that match their respective target site with 19-mer or 17-mer, with full match or with single mismatch to the target sequences in the secondary species. About 553 to 847 siRNAs were predicted without considering specificity. About 108-221 siRNAs were predicted to be specific in human and about 84 to 173 siRNAs were predicted to be specific in both human and NHPs (rhesus and cynomolgus monkey).

TABLE 7
CCR2 transcripts for cross-reactivity analysis

| | | Number of 19- | | |
|---|---|---|---|---|
| | | mers cross- | | |
| | Transcript | reactive with | 19-mers in | |
| Species | (Accession/ID) | NM_001123041.2 | common | Type |
| Human | NM_001123041.2 | 2650 | 962 | mRNA |
| | NM_001123396.1 | 1402 | | mRNA |
| | ENS011534069.1 | 2235 | | mRNA |
| | ENST00000445132 | 1402 | | Known protein coding |
| | ENST00000400888 | 2204 | | Known protein coding |
| | ENST00000292301 | 2643 | | Known protein coding |
| | ENST00000421659 | 433 | | Known protein coding |
| | ENST00000465202 | 625 | | Known processed transcript |
| Rhesus | By prediction | 1181 | 1181 | By prediction |
| | NM_001032806.1 | 537 | | mRNA |
| | ENSMMUT00000020033 | 616 | | Known by projection_protein coding |
| Cynomolgus | XM_005546901.2 | 1196 | 1196 | mRNA |
| Mouse | NM_009915.2 | 36 | 36 | mRNA |
| | XM_006512428.2 | 36 | | mRNA |
| | XM_011243063.1 | 56 | | mRNA |
| | XM_011243064.2 | 36 | | mRNA |
| | ENSMUST00000171719 | 53 | | Known protein coding |
| | ENSMUST00000168841 | 36 | | Known protein coding |
| | ENSMUST00000055918 | 36 | | Known protein coding |
| | ENSMUST00000165984 | 36 | | Known protein coding |
| Rat | NM_021866.1 | 35 | 35 | mRNA |
| | ENSRNOT00000078529 | 0 | | Known protein coding |
| Dog | XM_005632629.1 | 47 | 36 | mRNA |
| | ENS005632630.2 | 46 | | mRNA |
| | XM_005632631.2 | 46 | | mRNA |
| | ENSCAFT00000021891 | 36 | | Known by projection_protein coding |
| Pig | NM_001001619.1 | 75 | 75 | mRNA |
| | ENSSSCT00000028261 | 75 | | Known protein coding |
| Rabbit | XM_002713280.3 | 53 | 53 | mRNA |

A separate specificity analysis of siRNA candidates for each species was also performed. Through this analysis, 962 siRNA candidates directed against human CCR2 were analyzed. 274 antisense strands were specific in humans and 896 sense strands only have minimal specificity in humans. Among all siRNA candidates, 262 siRNA candidates were specific in humans and 108 of 553 rhesus and cynomolgus (i.e., non-human primates) cross-reactive siRNAs (human X NHP) were specific in humans.

553 siRNAs were analyzed for predicted specificity in NHP. It was found that 146 antisense strands were specific in NHP and 512 sense strands only have minimal specificity in NHP. Among all siRNA candidates, 138 siRNA candidates were specific in humans and showed humans and NHP cross-reactivity.

Another specificity analysis between humans and NHP was also performed. In the analysis of 19-mer, 84 siRNAs out of the total 553 siRNAs were specific in human and NHP. 4 siRNAs were highly specific in human. In the analysis with 17-mer (positions 2-18 of 19-mer), 90 siRNAs out of the total 581 siRNAs were specific in human and NHP. 6 siRNAs were highly specific in human.

These siRNAs can be further filtered according to the specificity criteria (e.g., absence of human miRNA seeds, absence of rhesus miRNA seeds, absence of conserved miRNA seeds among human, mouse and rat; off target frequency and two-or-more mismatches) and predicted siRNA activity.

siRNA Activity Prediction

Selected siRNA candidates were further evaluated for predicted siRNA activity. In order to obtain the specific activity, siRNAs with target sites that are abundant with SNPs (single nucleotide polymorphisms) were excluded. Human SNPs were mapped to siRNA target sites in the CCR2 transcript and analyzed. siRNA candidates for which the target sites were free from SNPs were selected.

The siRNA activity was also predicted based on selected siRNA chemistry and other algorithms. The siRNA candidate that is predicted to most likely be inactive siRNA is removed from the evaluation list.

siRNA Selection

Selected siRNA candidates after evaluation were listed in Table 2. The location of the target site for each siRNA molecule on human CCR2 mRNA (SEQ ID NO: 1) is also indicated in Table 2. The selected siRNAs target the coding region of human CCR2 mRNA.

### Example 2: Identification of Candidate siRNAs Targeting CSF1R

A similar approach to the identification of siRNA candidates targeting CCR2 was used to identify siRNA candidates targeting human CSF1R.

Human CSF1R mRNA sequence (Gene Bank NO. NM_005211.3; SEQ ID NO: 2) was used as the target template. All possible 19-mer siRNA molecules were created from this reference sequence. For all the siRNA candidates, target specificity, intra- and inter-species cross-activity, activity and other key features were evaluated.

The off-target genes for each siRNA strand were predicted for human, rhesus monkey, cynomolgus monkey. A specific score according to the off-target frequency was assigned to each siRNA strand. All the siRNA strands were analyzed for presence of human, rhesus monkey, dog, pig, rat, and mouse miRNA seed regions. Each siRNA candidate was then assigned to a specificity category in consideration of both the specificity score and miRNA seed analysis (as shown in Table 6).

The inter- and intra-species cross-reactivity of siRNA candidates were then calculated for transcript variants and different species, for 19-mers and 17-mers (nucleotides 2-18 of 19-mer), and for 19-mers and 17-mers with a single mismatch. About 1770 to 2957 siRNAs were predicted without considering specificity. About 623 to 1051 siRNAs were predicted to be specific in human and about 444 to 771 siRNAs were predicted to be specific in both human and NHPs (rhesus and cynomolgus monkey). Sequences including transcript variants from different species analyzed for cross-reactivity were in Table 8.

TABLE 8
CSF1R transcripts for cross-reactivity analysis

| | | Number of 19-mers | 19-mers | |
|---|---|---|---|---|
| | Transcript | cross-reactive with | in | Type |
| Species | (Accession/ID) | NM_005211.3 | common | |
| Human | NM_005211.3 | 3977 | 3860 | mRNA |
| | NM_001288705.2 | 3866 | | mRNA |
| | NM_001349736.1 | 3866 | | mRNA |
| | ENST00000286301 | 3971 | | Known protein coding |
| | NR_109969.1 | 3688 | | non-coding RNA |
| | ENST00000504875 | 3664 | | Known nonsense mediated decay |
| | ENST00000509861 | 478 | | Known retained intron |
| | ENST00000515068 | 499 | | Known nonsense mediated decay |
| | ENST00000515239 | 411 | | Known processed transcript |
| | ENST00000513609 | 396 | | Known protein coding |
| | ENST00000502660 | 998 | | Known protein coding |
| | ENST00000511344 | 620 | | Known protein coding |
| | ENST00000543093 | 887 | | Known protein coding |
| Rhesus | XM_001107711.3 | 1878 | 1878 | mRNA |
| | ENSMMUT00000016183 | 1598 | | Known by projection_protein coding |
| | ENSMMUT00000040845 | 1430 | | Known by projection_protein coding |
| | ENSMMUT00000064465 | 333 | | Known by projection_protein coding |
| Cyno | XM_005558240.2 | 1933 | 1849 | mRNA |
| | XM_015452130.1 | 1849 | | mRNA |
| Mouse | NM_001037859.2 | 115 | 115 | mRNA |
| | XM_006525584.1 | 115 | | mRNA |
| | XM_006525585.3 | 115 | | mRNA |
| | XM_006525586.3 | 115 | | mRNA |
| | XM_017317810.1 | 115 | | mRNA |
| | ENSMUST00000025523 | 115 | | Known protein coding |
| | ENSMUST00000115268 | 115 | | Known protein coding |
| | XR_001782332.1 | 109 | | misc_RNA |
| Rat | NM_001029901.1 | 105 | 102 | mRNA |
| | XM_006254813.3 | 108 | | mRNA |
| | XM_008772147.2 | 108 | | mRNA |
| | XM_008772148.2 | 108 | | mRNA |
| | ENSRNOT00000079360 | 105 | | Known protein coding |
| | ENSRNOT00000049357 | 105 | | Known protein coding |
| | ENSRNOT00000088988 | 22 | | Known protein coding |
| Dog | XM_546306.5 | 304 | 274 | mRNA |
| | ENSCAFT00000028942 | 309 | | Known by projection_protein coding |
| Pig | XM_003124100.5 | 222 | | mRNA |
| | XM_013995332.1 | 222 | 214 | mRNA |
| | ENSSSCT00000015787 | 214 | | Novel_protein coding |
| Rabbit | XM_017341189.1 | 220 | 218 | mRNA |
| | ENSOCUT00000021641 | 224 | | Known by projection_protein coding |

A separate specificity analysis of siRNA candidates to CSF1R for each species was also performed. Through the analysis, 3860 siRNA candidates directed against human CSF1R were analyzed. 1493 antisense strands were specific in humans, and 3504 sense strands only have minimal specificity in humans. Among all siRNA candidates, 1418 siRNA candidates were specific in humans and 623 of 1770 rhesus and cynomolgus cross-reactive siRNAs (human and NHP) were specific in humans.

siRNAs were analyzed for predicted specificity in NHP. It was found that 691 antisense strands were specific in NHP and 1636 sense strands only have minimal specificity in NHP. Among all siRNA candidates, 655 siRNA candidates were specific in humans and showed human and NHP cross-reactivity.

Another specificity analysis between humans and NHP was also performed. In the analysis of 19-mer, 444 siRNAs out of the total 1770 siRNAs were specific in human and NHP. 21 siRNAs were highly specific in humans. In the analysis with 17-mers (positions 2-18 of 19-mer), 481 siRNAs out of the total 1901 siRNAs were specific in humans and NHP. 23 siRNAs were highly specific in humans.

These siRNAs can be further filtered according to the specificity criteria (e.g., absence of human miRNA seeds, absence of rhesus miRNA seeds, absence of conserved miRNA seeds among human, mouse and rat; off target frequency and two-or-more mismatches) and predicted siRNA activity.

Selected siRNA candidates were further evaluated for predicted siRNA activity. In order to obtain the specific activity, siRNAs with target sites that are abundant with SNPs will be excluded. Human SNPs were mapped to siRNA target sites in the CSF1R transcript and analyzed. siRNA candidates for which the target sites were free from SNPs were selected. The siRNA activity was also predicted based on selected siRNA chemistry and other algorithms. The siRNA candidate that is predicted most likely to be inactive siRNA is removed from the evaluation list.

The selected siRNA candidates specific to CSF1R after evaluation were listed in Table 3 and modified siRNA strands are in Table 4. The location of the target site for each siRNA molecule on human CSF1R mRNA (SEQ ID NO: 2) is also indicated in the Tables. The selected siRNAs target both the coding region and 3′ UTR region of human CSF1R mRNA.

### Example 3: CSF1R siRNA Screening

Dual Dose Screening of CSF1R siRNA Duplexes

Human monocytic THP-1 cells were cultured and maintained in 96-well plates at a density of 25,000 cells per well. THP-1 monocytes were transfected with CSF1R siRNA duplexes (see Table 9) using Lipofectomine® 2000 (0.5 μl/well). The CSF1R siRNAs were transfected at a final concentration of 0.2 nM and 20 nM, respectively. An anti-Aha1 siRNA (XD-00033) was transfected as a positive control. Two scramble siRNA sequences (XD-00379 and XD-00385) were used as negative control. After incubating for 24 hours, the treated cells were harvested and the remaining CSF1R mRNA level was measured (Table 10).

TABLE 9
CSF1R siRNA duplexes and sequences

| Duplex- | | SEQ ID | | SEQ ID |
|---|---|---|---|---|
| ID | Sense Sequence | NO: | Antisense Sequence | NO |
| XD-08903 | ggGAAucccAGuGauAGAGdTsdT | 368 | CUCuAUcACUGGGAUUCCCdTsdT | 487 |
| XD-08904 | uuGcGAuGuGuGGgcAAuGdTsdT | 369 | cAUUGCCcAcAcAUCGcAAdTsdT | 488 |
| XD-08905 | gcGAuGuGuGGGcaAuGGcdTsdT | 370 | GCcAUUGCCcAcAcAUCGCdTsdT | 489 |
| XD-08906 | caAcGcuAccuuccAAAAcdTsdT | 371 | GUUUUGGAAGGuAGCGUUGdTsdT | 490 |
| XD-08907 | acGcuAccuuccAaAAcAcdTsdT | 372 | GUGUUUUGGAAGGuAGCGUdTsdT | 491 |
| XD-08908 | cgcuAccuuccAAaAcAcGdTsdT | 373 | CGUGUUUUGGAAGGuAGCGdTsdT | 492 |
| XD-08909 | ugcccGGcccuGGaAcGuGdTsdT | 374 | cACGUUCcAGGGCCGGGcAdTsdT | 493 |
| XD-08910 | ugcucAcAGAcccgGuGcudTsdT | 375 | AGcACCGGGUCUGUGAGcAdTsdT | 494 |
| XD-08911 | agGcGucucGcuGguGcGudTsdT | 376 | ACGcACcAGCGAGACGCCUdTsdT | 495 |
| XD-08912 | ucAAuGcAGuGcccuGAuGdTsdT | 377 | cAUcAGGGcACUGcAUUGAdTsdT | 496 |
| XD-08913 | caGcAuccGGcuGaAAGuGdTsdT | 378 | cACUUUcAGCCGGAUGCUGdTsdT | 497 |
| XD-08914 | caGAGcuGGuGcGgAuucGdTsdT | 379 | CGAAUCCGcACcAGCUCUGdTsdT | 498 |
| XD-08915 | gaGcuGGuGcGGAuucGAGdTsdT | 380 | CUCGAAUCCGcACcAGCUCdTsdT | 499 |
| XD-08916 | ugcGGAuucGAGGgGAGGcdTsdT | 381 | GCCUCCCCUCGAAUCCGcAdTsdT | 500 |
| XD-08917 | cuGcccAGAucGuguGcucdTsdT | 382 | GAGcAcACGAUCUGGGcAGdTsdT | 501 |
| XD-08918 | gcccAGAucGuGugcucAGdTsdT | 383 | CUGAGcAcACGAUCUGGGCdTsdT | 502 |
| XD-08919 | caGAucGuGuGcucAGccAdTsdT | 384 | UGGCUGAGcAcACGAUCUGdTsdT | 503 |
| XD-08920 | auAAccGuuAccAaAAAGudTsdT | 385 | ACUUUUUGGuAACGGUuAUdTsdT | 504 |
| XD-08921 | aaccGuuAccAAAaAGuccdTsdT | 386 | GGACUUUUUGGuAACGGUUdTsdT | 505 |
| XD-08922 | accGuuAccAAAAaGuccudTsdT | 387 | AGGACUUUUUGGuAACGGUdTsdT | 506 |
| XD-08923 | uuAccAAAAAGuccuGAccdTsdT | 388 | GGUcAGGACUUUUUGGuAAdTsdT | 507 |
| XD-08924 | agGuuuuAAcuGGaccuAcdTsdT | 389 | GuAGGUCcAGUuAAAACCUdTsdT | 508 |
| XD-08925 | gguuuuAAcuGGAccuAccdTsdT | 390 | GGuAGGUCcAGUuAAAACCdTsdT | 509 |
| XD-08926 | uuuAAcuGGAccuaccuGGdTsdT | 391 | CcAGGuAGGUCcAGUuAAAdTsdT | 510 |
| XD-08927 | uuAAcuGGAccuAccuGGGdTsdT | 392 | CCcAGGuAGGUCcAGUuAAdTsdT | 511 |
| XD-08928 | gaccuAccuGGGAcccuuudTsdT | 393 | AAAGGGUCCcAGGuAGGUCdTsdT | 512 |
| XD-08929 | ugAcGuuuGAGcucAcccudTsdT | 394 | AGGGUGAGCUcAAACGUcAdTsdT | 513 |
| XD-08930 | gacGuuuGAGcucacccuudTsdT | 395 | AAGGGUGAGCUcAAACGUCdTsdT | 514 |
| XD-08931 | uuGAGcucAcccuucGAuAdTsdT | 396 | uAUCGAAGGGUGAGCUcAAdTsdT | 515 |
| XD-08932 | agcucAcccuucGauAcccdTsdT | 397 | GGGuAUCGAAGGGUGAGCUdTsdT | 516 |
| XD-08933 | uucGAuAccccccaGAGGudTsdT | 398 | ACCUCUGGGGGGuAUCGAAdTsdT | 517 |
| XD-08934 | gauAccccccAGAgGuAAGdTsdT | 399 | CUuACCUCUGGGGGGuAUCdTsdT | 518 |
| XD-08935 | gaGAccuuAGAGcacAAccdTsdT | 400 | GGUUGUGCUCuAAGGUCUCdTsdT | 519 |
| XD-08936 | gaccuuAGAGcAcaAccAAdTsdT | 401 | UUGGUUGUGCUCuAAGGUCdTsdT | 520 |
| XD-08937 | caGcGuGGGGAGugGcuccdTsdT | 402 | GGAGCcACUCCCcACGCUGdTsdT | 521 |
| XD-08938 | aucccccGGAuGAguuccudTsdT | 403 | AGGAACUcAUCCGGGGGAUdTsdT | 522 |
| XD-08939 | cccGGAuGAGuuccucuucdTsdT | 404 | GAAGAGGAACUcAUCCGGGdTsdT | 523 |
| XD-08940 | guGGucGccuGcAuGuccAdTsdT | 405 | UGGAcAUGcAGGCGACcACdTsdT | 524 |
| XD-08941 | ugcuAuuGuAcAAguAuAAdTsdT | 406 | UuAuACUUGuAcAAuAGcAdTsdT | 525 |
| XD-08942 | caGGuccGcuGGAaGAucAdTsdT | 407 | UGAUCUUCcAGCGGACCUGdTsdT | 526 |
| XD-08943 | gguccGcuGGAAGaucAucdTsdT | 408 | GAUGAUCUUCcAGCGGACCdTsdT | 527 |
| XD-08944 | uccGcuGGAAGAucAucGAdTsdT | 409 | UCGAUGAUCUUCcAGCGGAdTsdT | 528 |
| XD-08945 | ccGcuGGAAGAucaucGAGdTsdT | 410 | CUCGAUGAUCUUCcAGCGGdTsdT | 529 |
| XD-08946 | aucAucGAGAGcuauGAGGdTsdT | 411 | CCUcAuAGCUCUCGAUGAUdTsdT | 530 |
| XD-08947 | caucGAGAGcuAugAGGGcdTsdT | 412 | GCCCUcAuAGCUCUCGAUGdTsdT | 531 |
| XD-08948 | uauGAGGGcAAcAguuAuAdTsdT | 413 | uAuAACUGUUGCCCUcAuAdTsdT | 532 |
| XD-08718 | agGGcAAcAGuuAuAcuuudTsdT | 414 | AAAGuAuAACUGUUGCCCUdTsdT | 533 |
| XD-08950 | ggcAAcAGuuAuAcuuucAdTsdT | 415 | UGAAAGuAuAACUGUUGCCdTsdT | 534 |
| XD-08751 | cacGcAGcuGccuuAcAAcdTsdT | 416 | GUUGuAAGGcAGCUGCGUGdTsdT | 535 |
| XD-08952 | uucccccGGAAcAaccuGcdTsdT | 417 | GcAGGUUGUUCCGGGGGAAdTsdT | 536 |
| XD-08953 | ccGGAAcAAccuGcAGuuudTsdT | 418 | AAACUGcAGGUUGUUCCGGdTsdT | 537 |
| XD-08954 | uuuGGuAAGAcccucGGAGdTsdT | 419 | CUCCGAGGGUCUuACcAAAdTsdT | 538 |
| XD-08955 | ugGuAAGAcccucgGAGcudTsdT | 420 | AGCUCCGAGGGUCUuACcAdTsdT | 539 |
| XD-08956 | ugAAGuccAcGGcccAuGcdTsdT | 421 | GcAUGGGCCGUGGACUUcAdTsdT | 540 |
| XD-08957 | gaAGuccAcGGcccAuGcudTsdT | 422 | AGcAUGGGCCGUGGACUUCdTsdT | 541 |
| XD-08958 | ccuGuAcuGGucAucAcGGdTsdT | 423 | CCGUGAUGACcAGuAcAGGdTsdT | 542 |
| XD-08959 | cuGuAcuGGucAucAcGGAdTsdT | 424 | UCCGUGAUGACcAGuAcAGdTsdT | 543 |
| XD-08960 | uguAcuGGucAucacGGAGdTsdT | 425 | CUCCGUGAUGACcAGuAcAdTsdT | 544 |
| XD-08961 | uacuGGucAucAcgGAGuAdTsdT | 426 | uACUCCGUGAUGACcAGuAdTsdT | 545 |
| XD-08962 | ggucAucAcGGAGuAcuGudTsdT | 427 | AcAGuACUCCGUGAUGACCdTsdT | 546 |
| XD-08963 | aucAcGGAGuAcuguuGcudTsdT | 428 | AGcAAcAGuACUCCGUGAUdTsdT | 547 |
| XD-08964 | ucAcGGAGuAcuGuuGcuAdTsdT | 429 | uAGcAAcAGuACUCCGUGAdTsdT | 548 |
| XD-08965 | acGGAGuAcuGuugcuAuGdTsdT | 430 | cAuAGcAAcAGuACUCCGUdTsdT | 549 |
| XD-08966 | aguAcuGuuGcuAuGGcGAdTsdT | 431 | UCGCcAuAGcAAcAGuACUdTsdT | 550 |
| XD-08967 | uguuGcuAuGGcGaccuGcdTsdT | 432 | GcAGGUCGCcAuAGcAAcAdTsdT | 551 |
| XD-08968 | gcuAuGGcGAccugcucAAdTsdT | 433 | UUGAGcAGGUCGCcAuAGCdTsdT | 552 |
| XD-08969 | auGGcGAccuGcucAAcuudTsdT | 434 | AAGUUGAGcAGGUCGCcAUdTsdT | 553 |
| XD-08970 | gcucAAcuuucuGcGAAGGdTsdT | 435 | CCUUCGcAGAAAGUUGAGCdTsdT | 554 |
| XD-08971 | caAcuuucuaGAaGGAAGdTsdT | 436 | CUUCCUUCGcAGAAAGUUGdTsdT | 555 |
| XD-08972 | uucuGcGAAGGAAgGcuGAdTsdT | 437 | UcAGCCUUCCUUCGcAGAAdTsdT | 556 |
| XD-08973 | caGGGuGuGGAcAccuAuGdTsdT | 438 | cAuAGGUGUCcAcACCCUGdTsdT | 557 |
| XD-08974 | ggAcAAGGAGGAugGAcGGdTsdT | 439 | CCGUCcAUCCUCCUUGUCCdTsdT | 558 |
| XD-08975 | gcuuccAAGAAuugcAuccdTsdT | 440 | GGAUGcAAUUCUUGGAAGCdTsdT | 559 |
| XD-08976 | acuucGGGcuGGcuAGGGAdTsdT | 441 | UCCCuAGCcAGCCCGAAGUdTsdT | 560 |
| XD-08977 | cuGGcuAGGGAcAucAuGAdTsdT | 442 | UcAUGAUGUCCCuAGCcAGdTsdT | 561 |
| XD-08978 | ggcuAGGGAcAucauGAAudTsdT | 443 | AUUcAUGAUGUCCCuAGCCdTsdT | 562 |
| XD-08979 | uuuGAcuGuGucuacAcGGdTsdT | 444 | CCGUGuAGAcAcAGUcAAAdTsdT | 563 |
| XD-08980 | ugAcuGuGucuAcacGGuudTsdT | 445 | AACCGUGuAGAcAcAGUcAdTsdT | 564 |
| XD-08981 | acuGuGucuAcAcgGuucAdTsdT | 446 | UGAACCGUGuAGAcAcAGUdTsdT | 565 |
| XD-08982 | guGucuAcAcGGuucAGAGdTsdT | 447 | CUCUGAACCGUGuAGAcACdTsdT | 566 |
| XD-08983 | cuAcAcGGuucAGaGcGAcdTsdT | 448 | GUCGCUCUGAACCGUGuAGdTsdT | 567 |
| XD-08984 | cacGGuucAGAGcgAcGucdTsdT | 449 | GACGUCGCUCUGAACCGUGdTsdT | 568 |
| XD-08985 | gguucAGAGcGAcgucuGGdTsdT | 450 | CcAGACGUCGCUCUGAACCdTsdT | 569 |
| XD-08986 | uucAGAGcGAcGucuGGucdTsdT | 451 | GACcAGACGUCGCUCUGAAdTsdT | 570 |
| XD-08987 | cuuGGGcuGAAucccuAccdTsdT | 452 | GGuAGGGAUUcAGCCcAAGdTsdT | 571 |
| XD-08988 | ugAAcAGcAAGuucuAuAAdTsdT | 453 | UuAuAGAACUUGCUGUUcAdTsdT | 572 |
| XD-08989 | gcAAGuucuAuAAacuGGudTsdT | 454 | ACcAGUUuAuAGAACUUGCdTsdT | 573 |
| XD-08990 | agAAuAuAuAcAGcAucAudTsdT | 455 | AUGAUGCUGuAuAuAUUCUdTsdT | 574 |
| XD-08991 | agAGAGcGGGAcuauAccAdTsdT | 456 | UGGuAuAGUCCCGCUCUCUdTsdT | 575 |
| XD-08992 | gaGAGcGGGAcuAuAccAAdTsdT | 457 | UUGGuAuAGUCCCGCUCUCdTsdT | 576 |
| XD-08993 | agAGcGGGAcuAuaccAAudTsdT | 458 | AUUGGuAuAGUCCCGCUCUdTsdT | 577 |
| XD-08994 | gcGGGAcuAuAccaAucuGdTsdT | 459 | cAGAUUGGuAuAGUCCCGCdTsdT | 578 |
| XD-08995 | ggAcuAuAccAAucuGccGdTsdT | 460 | CGGcAGAUUGGuAuAGUCCdTsdT | 579 |
| XD-08996 | ugcuGcGAGcAAGgGGAuAdTsdT | 461 | uAUCCCCUUGCUCGcAGcAdTsdT | 580 |
| XD-08997 | gcGAGcAAGGGGAuAucGcdTsdT | 462 | GCGAuAUCCCCUUGCUCGCdTsdT | 581 |
| XD-08998 | cgAGcAAGGGGAuaucGccdTsdT | 463 | GGCGAuAUCCCCUUGCUCGdTsdT | 582 |
| XD-08999 | gauAucGcccAGcccuuGcdTsdT | 464 | GcAAGGGCUGGGCGAuAUCdTsdT | 583 |
| XD-09000 | agcccAAcAAcuAucAGuudTsdT | 465 | AACUGAuAGUUGUUGGGCUdTsdT | 584 |
| XD-09001 | gcccAAcAAcuAucAGuucdTsdT | 466 | GAACUGAuAGUUGUUGGGCdTsdT | 585 |
| XD-09002 | cccAAcAAcuAucaGuucudTsdT | 467 | AGAACUGAuAGUUGUUGGGdTsdT | 586 |
| XD-09003 | acAAAcucuGccuucGGucdTsdT | 468 | GACCGAAGGcAGAGUUUGUdTsdT | 587 |
| XD-09004 | ucuGccuucGGucauuucAdTsdT | 469 | UGAAAUGACCGAAGGcAGAdTsdT | 588 |
| XD-09005 | gccuucGGucAuuucAcucdTsdT | 470 | GAGUGAAAUGACCGAAGGCdTsdT | 589 |
| XD-09006 | ucGGucAuuucAcucAAcAdTsdT | 471 | UGUUGAGUGAAAUGACCGAdTsdT | 590 |
| XD-09007 | cucAuGGuGuuGGccucGudTsdT | 472 | ACGAGGCcAAcACcAUGAGdTsdT | 591 |
| XD-09008 | ucAuGGuGuuGGccucGuGdTsdT | 473 | cACGAGGCcAAcACcAUGAdTsdT | 592 |
| XD-09009 | cauGGuGuuGGccucGuGudTsdT | 474 | AcACGAGGCcAAcACcAUGdTsdT | 593 |
| XD-09010 | gguGuuGGccucGuGuuuGdTsdT | 475 | cAAAcACGAGGCcAAcACCdTsdT | 594 |
| XD-09011 | cguGuuuGcuAuGccAAcudTsdT | 476 | AGUUGGcAuAGcAAAcACGdTsdT | 595 |
| XD-09012 | agcuAAAAGuuGGgGGuGudTsdT | 477 | AcACCCCcAACUUUuAGCUdTsdT | 596 |
| XD-09013 | ccAAGcuGAcucAuccuAAdTsdT | 478 | UuAGGAUGAGUcAGCUUGGdTsdT | 597 |
| XD-09014 | agcuGAcucAuccuAAcuAdTsdT | 479 | uAGUuAGGAUGAGUcAGCUdTsdT | 598 |
| XD-09015 | cuGAcucAuccuAacuAAcdTsdT | 480 | GuuAGUuAGGAUGAGUcAGdTsdT | 599 |
| XD-09016 | ugAcucAuccuAAcuAAcAdTsdT | 481 | UGUuAGUuAGGAUGAGUcAdTsdT | 600 |
| XD-09017 | gacucAuccuAAcuAAcAGdTsdT | 482 | CUGUuAGUuAGGAUGAGUCdTsdT | 601 |
| XD-09018 | uccuAAcuAAcAGucAcGcdTsdT | 483 | GCGUGACUGUuAGUuAGGAdTsdT | 602 |
| XD-09019 | ccuAAcuAAcAGucAcGccdTsdT | 484 | GGCGUGACUGUuAGUuAGGdTsdT | 603 |
| XD-09020 | uccAcAuuAAAcuaAcAGcdTsdT | 485 | GCUGUuAGUUuAAUGUGGAdTsdT | 604 |
| XD-09021 | ccAcAuuAAAcuAacAGcAdTsdT | 486 | UGCUGUuAGUUuAAUGUGGdTsdT | 605 |

TABLE 10
CSF1R mRNA expression level after siRNA duplex treatment

| | Remaining | | Remaining | |
|---|---|---|---|---|
| SiRNA | CSF1R mRNA | | CSF1R mRNA | |
| Duplex ID | (20 nM) | SD | (0.2 nM) | SD |
| XD-09016 | 0.18537 | 0.02284 | 0.50702 | 0.06445 |
| XD-08982 | 0.18692 | 0.00529 | 0.78050 | 0.02888 |
| XD-08947 | 0.19355 | 0.01912 | 0.55900 | 0.06748 |
| XD-08993 | 0.19394 | 0.04830 | 0.58760 | 0.02935 |
| XD-08988 | 0.21506 | 0.01206 | 0.57405 | 0.06757 |
| XD-08944 | 0.21750 | 0.01495 | 0.62030 | 0.07051 |
| XD-08969 | 0.21805 | 0.01873 | 0.72911 | 0.04768 |
| XD-09003 | 0.22426 | 0.02053 | 0.73782 | 0.01199 |
| XD-09002 | 0.23038 | 0.00631 | 0.90398 | 0.07340 |
| XD-08917 | 0.23503 | 0.05984 | 0.80799 | 0.08275 |
| XD-08936 | 0.24159 | 0.01493 | 0.60641 | 0.05563 |
| XD-08923 | 0.24349 | 0.03950 | 0.69406 | 0.06145 |
| XD-09021 | 0.24489 | 0.02555 | 0.80475 | 0.07459 |
| XD-08989 | 0.25338 | 0.01847 | 0.82919 | 0.06989 |
| XD-08963 | 0.25451 | 0.01114 | 0.77794 | 0.04950 |
| XD-08986 | 0.25769 | 0.01899 | 0.71013 | 0.10820 |
| XD-08922 | 0.26956 | 0.05050 | 0.67210 | 0.05036 |
| XD-08985 | 0.27418 | 0.01579 | 0.81107 | 0.05929 |
| XD-08911 | 0.27698 | 0.02007 | 0.97347 | 0.05620 |
| XD-08975 | 0.27764 | 0.02523 | 0.80713 | 0.06685 |
| XD-08998 | 0.27953 | 0.02538 | 0.92203 | 0.05824 |
| XD-08920 | 0.28126 | 0.01955 | 0.88899 | 0.04191 |
| XD-08910 | 0.28392 | 0.04528 | 0.97103 | 0.05298 |
| XD-08919 | 0.28978 | 0.00295 | 0.82317 | 0.07477 |
| XD-09000 | 0.29223 | 0.02449 | 1.01798 | 0.07085 |
| XD-09005 | 0.29234 | 0.01560 | 0.80071 | 0.12501 |
| XD-09006 | 0.29309 | 0.01102 | 0.66257 | 0.06075 |
| XD-09015 | 0.29864 | 0.02154 | 0.77911 | 0.04748 |
| XD-08959 | 0.30378 | 0.01653 | 0.88676 | 0.05556 |
| XD-09020 | 0.30655 | 0.03732 | 0.91608 | 0.06192 |
| XD-08965 | 0.30676 | 0.01428 | 0.94295 | 0.08738 |
| XD-08983 | 0.30887 | 0.04038 | 0.95985 | 0.11313 |
| XD-09017 | 0.31064 | 0.01470 | 0.86726 | 0.07573 |
| XD-08718 | 0.31191 | 0.02699 | 0.89423 | 0.07456 |
| XD-08921 | 0.31315 | 0.03781 | 0.95873 | 0.10268 |
| XD-08964 | 0.31794 | 0.00603 | 0.87172 | 0.02964 |
| XD-08937 | 0.33454 | 0.04225 | 0.67101 | 0.06215 |
| XD-09014 | 0.33545 | 0.02391 | 0.82205 | 0.06377 |
| XD-08946 | 0.34265 | 0.01887 | 0.86198 | 0.07153 |
| XD-08970 | 0.34529 | 0.02865 | 1.03488 | 0.06560 |
| XD-08961 | 0.35079 | 0.02145 | 1.00360 | 0.07367 |
| XD-08929 | 0.35756 | 0.04288 | 0.95156 | 0.18035 |
| XD-08974 | 0.35893 | 0.01063 | 0.89774 | 0.08100 |
| XD-08906 | 0.35986 | 0.01554 | 0.97084 | 0.05452 |
| XD-09004 | 0.36750 | 0.02836 | 0.62993 | 0.04527 |
| XD-08954 | 0.37519 | 0.03992 | 0.84890 | 0.02625 |
| XD-09011 | 0.37644 | 0.03108 | 0.96006 | 0.09453 |
| XD-08908 | 0.37894 | 0.03524 | 1.00367 | 0.06173 |
| XD-08925 | 0.37905 | 0.09014 | 0.83044 | 0.08860 |
| XD-09001 | 0.37916 | 0.01949 | 1.11165 | 0.09949 |
| XD-08996 | 0.41014 | 0.02609 | 0.94661 | 0.02649 |
| XD-08945 | 0.41198 | 0.02662 | 0.95633 | 0.05070 |
| XD-08907 | 0.43809 | 0.03115 | 0.96289 | 0.06900 |
| XD-08930 | 0.44301 | 0.02980 | 0.98719 | 0.03229 |
| XD-08973 | 0.44496 | 0.05228 | 0.90093 | 0.10734 |
| XD-08976 | 0.44526 | 0.01723 | 0.82831 | 0.03961 |
| XD-08939 | 0.44655 | 0.02934 | 0.93025 | 0.04863 |
| XD-09018 | 0.46374 | 0.03399 | 0.88168 | 0.03309 |
| XD-08955 | 0.46474 | 0.04281 | 0.83119 | 0.08266 |
| XD-09013 | 0.48310 | 0.03093 | 0.96855 | 0.01648 |
| XD-08992 | 0.48411 | 0.03658 | 0.98462 | 0.09052 |
| XD-08977 | 0.48602 | 0.04398 | 0.95141 | 0.08327 |
| XD-08971 | 0.51621 | 0.02496 | 0.97747 | 0.09487 |
| XD-08914 | 0.52102 | 0.00757 | 0.97345 | 0.07494 |
| XD-08995 | 0.54138 | 0.01044 | 1.02092 | 0.08231 |
| XD-08991 | 0.55009 | 0.02018 | 1.04190 | 0.07506 |
| XD-08987 | 0.55465 | 0.01375 | 0.93050 | 0.10621 |
| XD-09008 | 0.55637 | 0.02834 | 1.03410 | 0.05269 |
| XD-09007 | 0.56457 | 0.05419 | 0.95311 | 0.04485 |
| XD-08950 | 0.57008 | 0.03851 | 0.94692 | 0.06396 |
| XD-08984 | 0.57905 | 0.03816 | 0.92953 | 0.04740 |
| XD-08980 | 0.58398 | 0.05652 | 1.02200 | 0.07496 |
| XD-08927 | 0.59037 | 0.04877 | 0.92002 | 0.17925 |
| XD-08994 | 0.59708 | 0.06231 | 0.97299 | 0.11487 |
| XD-08948 | 0.60345 | 0.01408 | 0.97558 | 0.04949 |
| XD-08943 | 0.61742 | 0.03261 | 0.88335 | 0.05723 |
| XD-09010 | 0.63971 | 0.06697 | 1.09204 | 0.13856 |
| XD-08979 | 0.65838 | 0.09571 | 1.01448 | 0.09057 |
| XD-08913 | 0.66500 | 0.04354 | 0.95628 | 0.06459 |
| XD-08960 | 0.67138 | 0.03943 | 1.08670 | 0.07317 |
| XD-08978 | 0.67992 | 0.06028 | 0.96962 | 0.06596 |
| XD-08718 | 0.68433 | 0.02467 | 0.94005 | 0.03550 |
| XD-09019 | 0.68810 | 0.05982 | 0.98429 | 0.04044 |
| XD-08931 | 0.71472 | 0.01747 | 1.02415 | 0.09088 |
| XD-08942 | 0.71682 | 0.06685 | 0.92023 | 0.04772 |
| XD-08967 | 0.71778 | 0.05235 | 1.11323 | 0.06628 |
| XD-08997 | 0.72882 | 0.01181 | 1.06092 | 0.05433 |
| XD-08968 | 0.73791 | 0.02149 | 1.03693 | 0.14572 |
| XD-08941 | 0.75432 | 0.04593 | 0.98308 | 0.04523 |
| XD-08972 | 0.76128 | 0.10513 | 0.95568 | 0.06176 |
| XD-08966 | 0.79411 | 0.03587 | 0.81351 | 0.13077 |
| XD-08953 | 0.80859 | 0.06608 | 0.90581 | 0.07752 |
| XD-08903 | 0.81277 | 0.05747 | 1.00663 | 0.04189 |
| XD-08904 | 0.81916 | 0.05336 | 1.00953 | 0.04823 |
| XD-08990 | 0.83319 | 0.06048 | 0.91711 | 0.07383 |
| XD-08909 | 0.83663 | 0.07080 | 0.96907 | 0.10826 |
| XD-08981 | 0.84219 | 0.15995 | 1.09766 | 0.11967 |
| XD-08918 | 0.84787 | 0.01734 | 0.95026 | 0.05807 |
| XD-08916 | 0.85213 | 0.04898 | 0.97463 | 0.06636 |
| XD-09009 | 0.85404 | 0.03917 | 1.15060 | 0.04359 |
| XD-08915 | 0.86321 | 0.12021 | 1.01780 | 0.18566 |
| XD-08956 | 0.86690 | 0.08694 | 0.85477 | 0.07408 |
| XD-08962 | 0.87373 | 0.05496 | 1.10057 | 0.02479 |
| XD-08957 | 0.90137 | 0.03356 | 0.88267 | 0.08385 |
| XD-08928 | 0.90659 | 0.10278 | 0.95260 | 0.20736 |
| XD-08933 | 0.92109 | 0.09930 | 0.91102 | 0.06753 |
| XD-08958 | 0.92751 | 0.02078 | 1.00619 | 0.06992 |
| XD-08926 | 0.92776 | 0.12489 | 0.91675 | 0.12077 |
| XD-08932 | 0.93011 | 0.10329 | 1.02311 | 0.09976 |
| XD-08912 | 0.93804 | 0.02806 | 1.02081 | 0.08995 |
| XD-08935 | 0.97156 | 0.06127 | 0.83523 | 0.06430 |
| XD-08924 | 0.98158 | 0.06688 | 0.89400 | 0.07711 |
| XD-08940 | 0.99113 | 0.01645 | 0.94643 | 0.01959 |
| XD-08934 | 0.99225 | 0.08202 | 0.89670 | 0.08148 |
| XD-08905 | 0.99864 | 0.05744 | 0.91416 | 0.14105 |
| XD-08938 | 1.02480 | 0.06030 | 0.82452 | 0.10330 |
| XD-08952 | 1.03108 | 0.06577 | 0.89369 | 0.03782 |
| XD-08999 | 1.11078 | 0.06150 | 1.08661 | 0.10997 |
| XD-00379 (Negative | 0.86933 | 0.02880 | 1.12239 | 0.02086 |
| Control) | | | | |
| XD-00385(Negative | 0.90707 | 0.03773 | 1.03351 | 0.03986 |
| control) | | | | |
| XD-00033 (Aha-1; | 0.18975 | 0.01787 | 0.66840 | 0.04754 |
| positive control) | | | | |

Dose Response of Selected CSF1R siRNA Duplexes

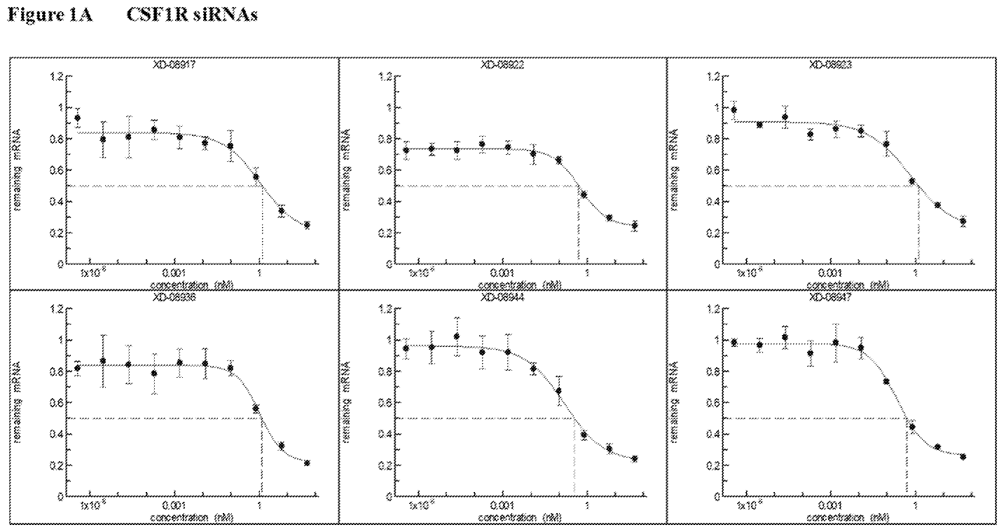

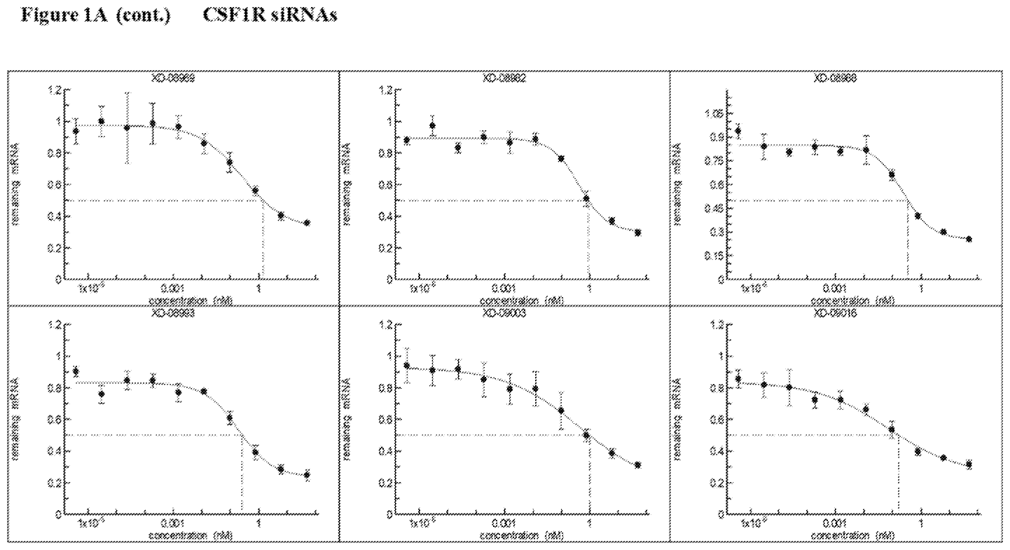

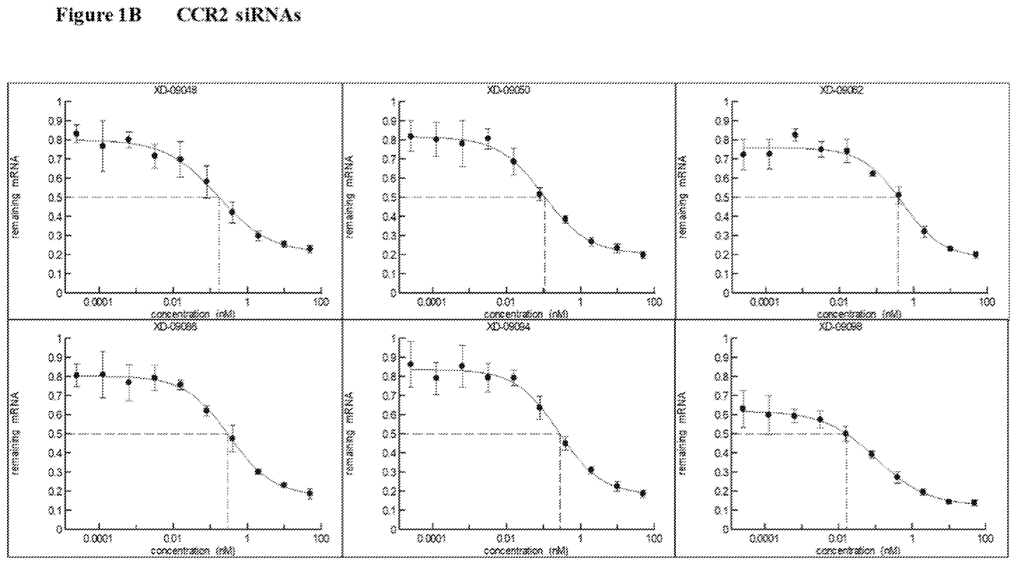

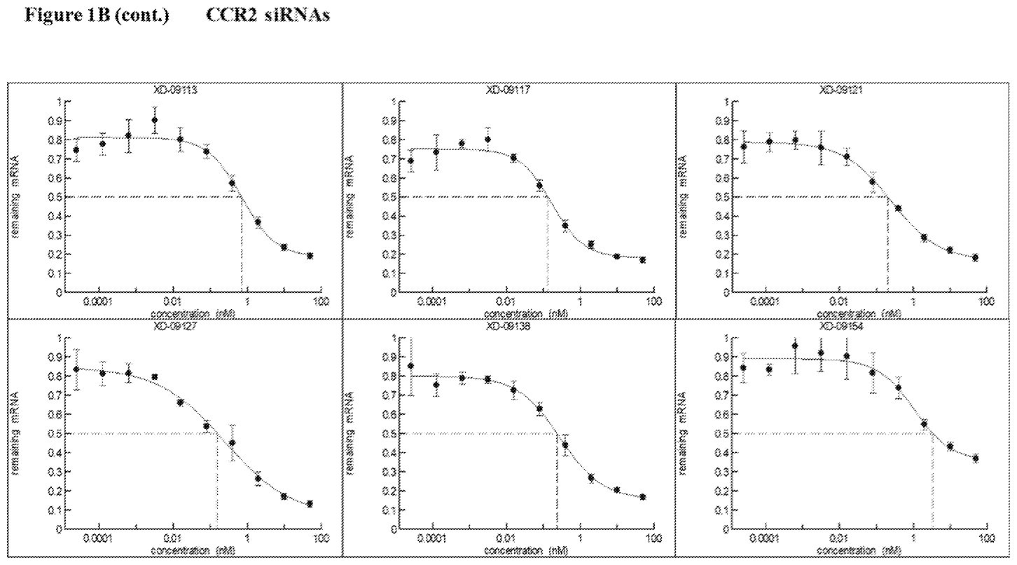

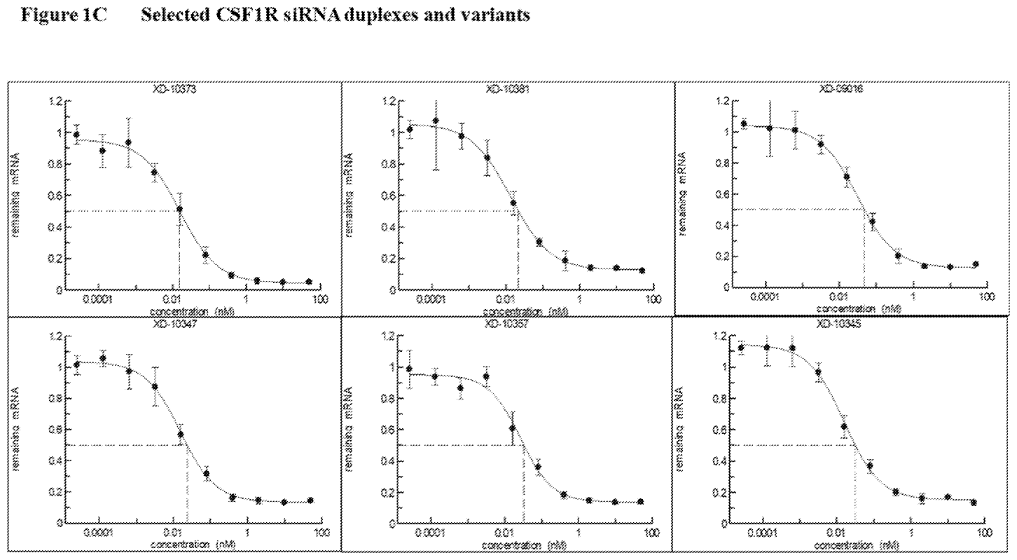

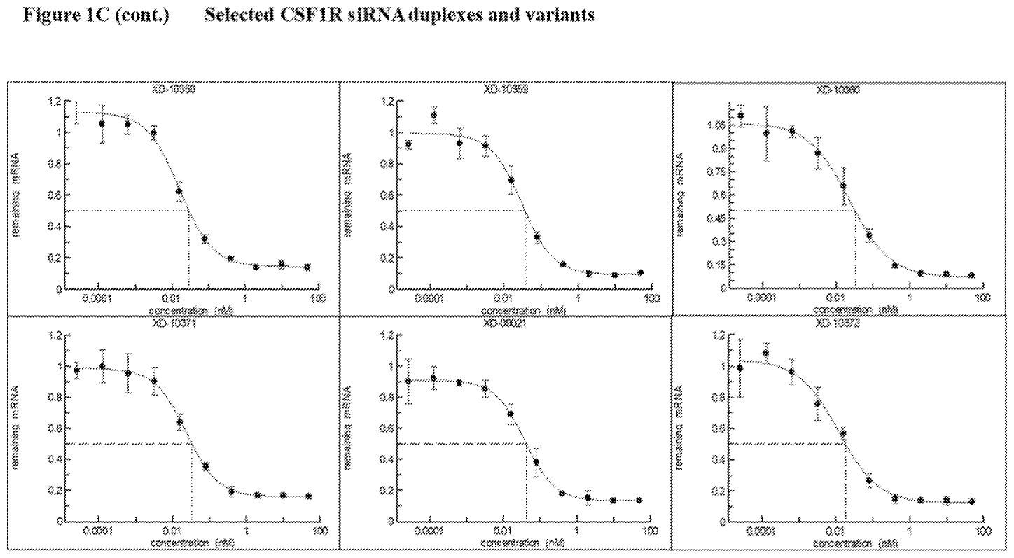

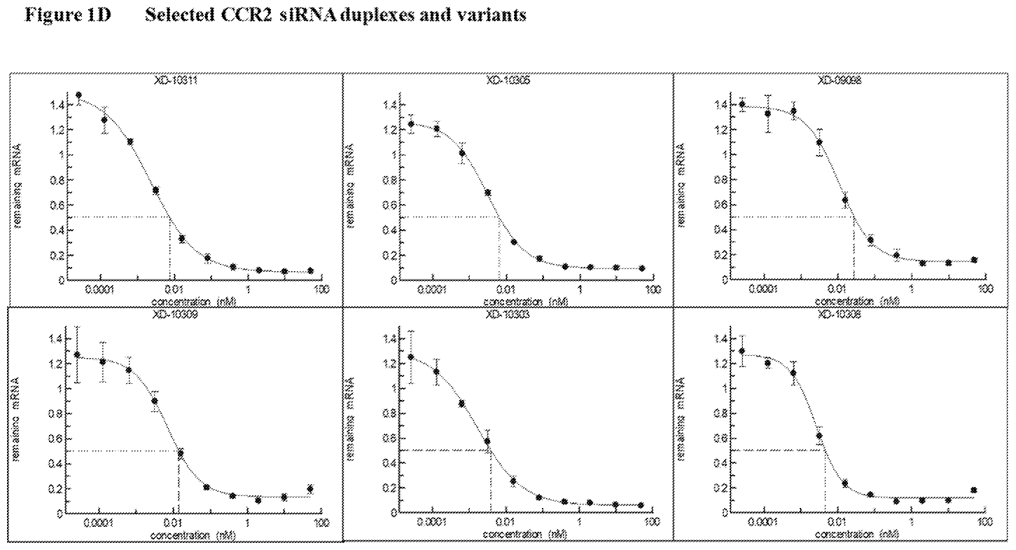

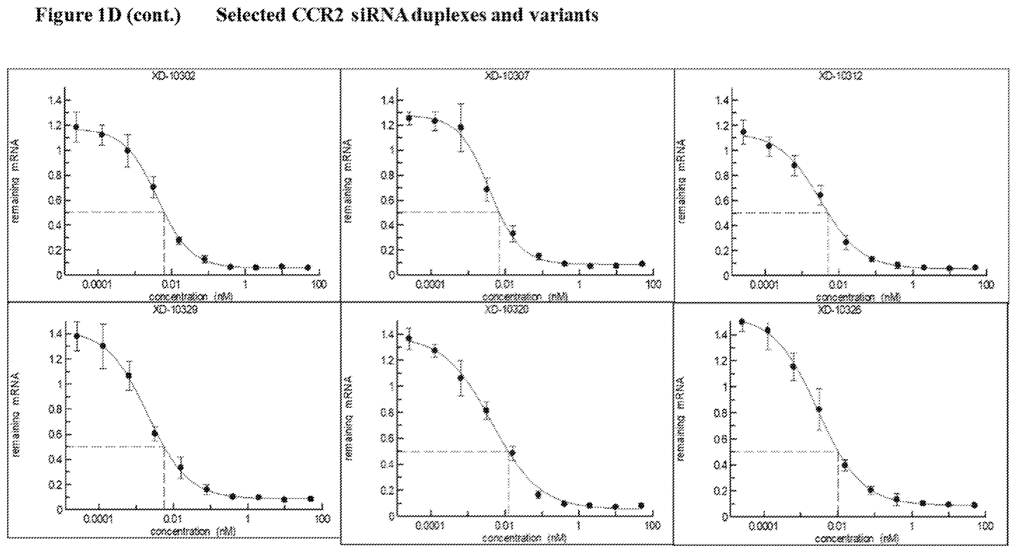

CSF1R siRNA duplexes that caused a significant reduction of CSF1R mRNA level in the dual dose screening were selected and further tested for the dose response. Human monocytic THP-1 cells were cultured and maintained in 96-well plates at a density of 25,000 cells per well. THP-1 monocytes were transfected with CSF1R siRNA duplexes selected from the previous dual dose screening at various concentrations using Lipofectomine® 2000 (0.5 μl/well). The doses for each CSF1R siRNA duplex included 50 nM, 6.25 nM, 0.78 nM, 1.2×10⁻² nM, 1.5×10⁻³ nM, 1.9×10⁻⁴ nM, 3.0×10⁻⁶ nM, and 3.7×10⁻⁷ nM. Following incubation of 24 hours, the treated cells were harvested and the remaining CSF1R mRNA level was measured in each condition. The IC₅₀ value of each CSF1R duplex was determined as shown in Table 11 and each dose response curve is shown in FIG. 1A.

TABLE 11
IC₅₀ of CSF1R siRNA duplexes

| siRNA Duplex ID | IC₅₀ (nM) |
|---|---|
| XD-08917 | 1.292 |
| XD-08922 | 0.494 |
| XD-08923 | 1.283 |
| XD-08936 | 1.251 |
| XD-08944 | 0.349 |
| XD-08947 | 0.495 |
| XD-08969 | 1.452 |
| XD-08982 | 0.906 |
| XD-08988 | 0.348 |
| XD-08993 | 0.267 |
| XD-09003 | 0.981 |
| XD-09016 | 0.167 |

### Example 4: CCR2 siRNA Screening

Dual Dose Screening of CCR2 siRNA Duplexes

Human monocytic THP-1 cells were cultured and maintained in 96-well plates at a density of 25,000 cells per well. THP-1 monocytes were transfected with CCR2 siRNA duplexes (Table 12) using Lipofectomine® 2000 (0.5 μl/well). The CCR2 siRNAs were transfected at a final concentration of 0.2 nM and 20 nM, respectively. An anti-Aha1 siRNA (XD-00033) was transfected as a positive control. Two scramble siRNA sequences (XD-00379 and XD-00385) were used as negative control. Following incubation of 24 hours, the treated cells were harvested and the remaining CCR2 mRNA level was measured (Table 13).

TABLE 12
CCR2 siRNA duplexes and sequences

| Duplex | | SEQ ID | | SEQ ID |
|---|---|---|---|---|
| ID | Sense Sequence | NO: | Antisense Sequence | NO: |
| XD-09027 | caAGGAcGcAuuuccccAGdTsdT | 606 | CUGGGGAAAUGCGUCCUUGdTsdT | 744 |
| XD-09028 | aaGGAcGcAuuuccccAGudTsdT | 607 | ACUGGGGAAAUGCGUCCUUdTsdT | 745 |
| XD-09029 | ggAcGcAuuuccccAGuAcdTsdT | 608 | GuACUGGGGAAAUGCGUCCdTsdT | 746 |
| XD-09030 | gacGcAuuuccccaGuAcAdTsdT | 609 | UGuACUGGGGAAAUGCGUCdTsdT | 747 |
| XD-09031 | acGcAuuuccccAguAcAudTsdT | 610 | AUGuACUGGGGAAAUGCGUdTsdT | 748 |
| XD-09032 | cgcAuuuccccAGuAcAucdTsdT | 611 | GAUGuACUGGGGAAAUGCGdTsdT | 749 |
| XD-09033 | cauuuccccAGuAcAuccAdTsdT | 612 | uGGAUGuACUGGGGAAAUGdTsdT | 750 |
| XD-09034 | auuuccccAGuAcauccAcdTsdT | 613 | GUGGAUGuACUGGGGAAAUdTsdT | 751 |
| XD-09035 | uuuccccAGuAcAuccAcAdTsdT | 614 | UGUGGAUGuACUGGGGAAAdTsdT | 752 |
| XD-09036 | uccccAGuAcAuccAcAAcdTsdT | 615 | GUUGUGGAUGuACUGGGGAdTsdT | 753 |
| XD-09037 | caGuAcAuccAcAacAuGcdTsdT | 616 | GcAUGUUGUGGAUGuACUGdTsdT | 754 |
| XD-09038 | guAcAuccAcAAcauGcuGdTsdT | 617 | cAGcAUGUUGUGGAUGuACdTsdT | 755 |
| XD-09039 | acAuccAcAAcAugcuGucdTsdT | 618 | GAcAGcAUGUUGUGGAUGUdTsdT | 756 |
| XD-09040 | auGcuGuccAcAucucGuudTsdT | 619 | AACGAGAUGUGGAcAGcAUdTsdT | 757 |
| XD-09041 | ugcuGuccAcAucucGuucdTsdT | 620 | GAACGAGAUGUGGAcAGcAdTsdT | 758 |
| XD-09042 | gcuGuccAcAucucGuucudTsdT | 621 | AGAACGAGAUGUGGAcAGCdTsdT | 759 |
| XD-09043 | cuGuccAcAucucguucucdTsdT | 622 | GAGAACGAGAUGUGGAcAGdTsdT | 760 |
| XD-09044 | guccAcAucucGuucucGGdTsdT | 623 | ccGAGAACGAGAUGUGGACdTsdT | 761 |
| XD-09045 | uccAcAucucGuucucGGudTsdT | 624 | ACCGAGAACGAGAUGUGGAdTsdT | 762 |
| XD-09046 | ccAcAucucGuucucGGuudTsdT | 625 | AACCGAGAACGAGAUGUGGdTsdT | 763 |
| XD-09047 | cacAucucGuucucGGuuudTsdT | 626 | AAACCGAGAACGAGAUGUGdTsdT | 764 |
| XD-09048 | acAucucGuucucgGuuuAdTsdT | 627 | uAAACCGAGAACGAGAUGUdTsdT | 765 |
| XD-09049 | caucucGuucucGguuuAudTsdT | 628 | AuAAACCGAGAACGAGAUGdTsdT | 766 |
| XD-09050 | ucucGuucucGGuuuAucAdTsdT | 629 | UGAuAAACCGAGAACGAGAdTsdT | 767 |
| XD-09051 | cucGuucucGGuuuAucAGdTsdT | 630 | CUGAuAAACCGAGAACGAGdTsdT | 768 |
| XD-09052 | ucGuucucGGuuuaucAGAdTsdT | 631 | UCUGAuAAACCGAGAACGAdTsdT | 769 |
| XD-09053 | cguucucGGuuuAucAGAAdTsdT | 632 | UUCUGAuAAACCGAGAACGdTsdT | 770 |
| XD-09054 | uucucGGuuuAucaGAAAudTsdT | 633 | AUUUCUGAuAAACCGAGAAdTsdT | 771 |
| XD-09055 | ucucGGuuuAucAgAAAuAdTsdT | 634 | uAUUUCUGAuAAACCGAGAdTsdT | 772 |
| XD-09056 | cucGGuuuAucAGaAAuAcdTsdT | 635 | GuAUUUCUGAuAAACCGAGdTsdT | 773 |
| XD-09057 | ucGGuuuAucAGAaAuAccdTsdT | 636 | GGuAUUUCUGAuAAACCGAdTsdT | 774 |
| XD-09058 | uuuAucAGAAAuAccAAcGdTsdT | 637 | CGUUGGuAUUUCUGAuAAAdTsdT | 775 |
| XD-09059 | agAGcGGuGAAGAaGucAcdTsdT | 638 | GUGACUUCUUcACCGCUCUdTsdT | 776 |
| XD-09060 | cgGuGAAGAAGucaccAccdTsdT | 630 | GGUGGUGACUUCUUcACCGdTsdT | 777 |
| XD-09061 | aaGucAccAccuuuuuuGAdTsdT | 640 | UcAAAAAAGGUGGUGACUUdTsdT | 778 |
| XD-09062 | ucAccAccuuuuuuGAuuAdTsdT | 641 | uAAUcAAAAAAGGUGGUGAdTsdT | 779 |
| XD-09063 | caccAccuuuuuugAuuAudTsdT | 642 | AuAAUcAAAAAAGGUGGUGdTsdT | 780 |
| XD-09064 | accAccuuuuuuGauuAuGdTsdT | 643 | cAuAAUcAAAAAAGGUGGUdTsdT | 781 |
| XD-09065 | uuuuGAuuAuGAuuAcGGudTsdT | 644 | ACCGuAAUcAuAAUcAAAAdTsdT | 782 |
| XD-09066 | ugAuuAuGAuuAcgGuGcudTsdT | 645 | AGcACCGuAAUcAuAAUcAdTsdT | 783 |
| XD-09067 | uuAuGAuuAcGGugcucccdTsdT | 646 | GGGAGcACCGuAAUcAuAAdTsdT | 784 |
| XD-09068 | uauGAuuAcGGuGcucccudTsdT | 647 | AGGGAGcACCGuAAUcAuAdTsdT | 785 |
| XD-09069 | ugAuuAcGGuGcucccuGudTsdT | 648 | AcAGGGAGcACCGuAAUcAdTsdT | 786 |
| XD-09070 | gauuAcGGuGcucccuGucdTsdT | 649 | GAcAGGGAGcACCGuAAUCdTsdT | 787 |
| XD-09071 | auuAcGGuGcucccuGucAdTsdT | 650 | UGAcAGGGAGcACCGuAAUdTsdT | 788 |
| XD-09072 | uuAcGGuGcucccuGucAudTsdT | 651 | AUGAcAGGGAGcACCGuAAdTsdT | 789 |
| XD-09073 | acGGuGcucccuGucAuAAdTsdT | 652 | UuAUGAcAGGGAGcACCGUdTsdT | 790 |
| XD-09074 | guGcucccuGucAuAAAuudTsdT | 653 | AAUUuAUGAcAGGGAGcACdTsdT | 701 |
| XD-09075 | ugcucccuGucAuaAAuuudTsdT | 654 | AAAuUuAUGAcAGGGAGcAdTsdT | 792 |
| XD-09076 | cucccuGucAuAAauuuGAdTsdT | 655 | UcAAAUUuAUGAcAGGGAGdTsdT | 793 |
| XD-09077 | ccuGucAuAAAuuuGAcGudTsdT | 656 | ACGUcAAAUUuAUGAcAGGdTsdT | 794 |
| XD-09078 | ugucAuAAAuuuGacGuGAdTsdT | 657 | UcACGUcAAAUUuAUGAcAdTsdT | 795 |
| XD-09079 | gucAuAAAuuuGAcGuGAAdTsdT | 658 | UUcACGUcAAAUUuAUGACdTsdT | 796 |
| XD-09080 | ucAuAAAuuuGAcguGAAGdTsdT | 659 | CUUcACGUcAAAUUuAUGAdTsdT | 797 |
| XD-09081 | auAAAuuuGAcGugAAGcAdTsdT | 660 | UGCUUcACGUcAAAUUuAUdTsdT | 798 |
| XD-09082 | aauuuGAcGuGAAgcAAAudTsdT | 661 | AUUUGCUUcACGUcAAAUUdTsdT | 799 |
| XD-09083 | auuuGAcGuGAAGcAAAuudTsdT | 662 | AAUUUGCUUcACGUcAAAUdTsdT | 800 |
| XD-09084 | uuuGAcGuGAAGcaAAuuGdTsdT | 663 | cAAUUUGCUUcACGUcAAAdTsdT | 801 |
| XD-09085 | uuGAcGuGAAGcAaAuuGGdTsdT | 664 | CcAAUUUGCUUcACGUcAAdTsdT | 802 |
| XD-09086 | ugAcGuGAAGcAAauuGGGdTsdT | 665 | CCcAAUUUGCUUcACGUcAdTsdT | 803 |
| XD-09087 | gacGuGAAGcAAAuuGGGGdTsdT | 666 | CCCcAAUUUGCUUcACGUCdTsdT | 804 |
| XD-09088 | acGuGAAGcAAAuuGGGGcdTsdT | 667 | GCCCcAAUUUGCUUcACGUdTsdT | 805 |
| XD-09089 | cg-uGAAGcAAAuugGGGccdTsdT | 668 | GGCCCcAAUUUGCUUcACGdTsdT | 806 |
| XD-09090 | gaAGcAAAuuGGGgcccAAdTsdT | 669 | UUGGGCCCcAAUUUGCUUCdTsdT | 807 |
| XD-09091 | aaGcAAAuuGGGGcccAAcdTsdT | 670 | GUUGGGCCCcAAUUUGCUUdTsdT | 808 |
| XD-09092 | agcAAAuuGGGGcccAAcudTsdT | 671 | AGUUGGGCCCcAAUUUGCUdTsdT | 809 |
| XD-09093 | gcAAAuuGGGGcccAAcucdTsdT | 672 | GAGUUGGGCCCcAAUUUGCdTsdT | 810 |
| XD-09094 | ugcAAAAAGcuGAaGuGcudTsdT | 673 | AGcACUUcAGCUUUUUGcAdTsdT | 811 |
| XD-09095 | aaAGcuGAAGuGcuuGAcudTsdT | 674 | AGUcAAGcACUUcAGCUUUdTsdT | 812 |
| XD-09096 | ugcuuGAcuGAcAuuuAccdTsdT | 675 | GGuAAAUGUcAGUcAAGcAdTsdT | 813 |
| XD-09097 | cuuGAcuGAcAuuuAccuGdTsdT | 676 | cAGGuAAAUGUcAGUcAAGdTsdT | 814 |
| XD-09098 | ugcuuuuucuuAuuAcucudTsdT | 677 | AGAGuAAuAAGAAAAAGcAdTsdT | 815 |
| XD-09099 | uucuuAuuAcucucccAuudTsdT | 678 | AAUGGGAGAGuAAuAAGAAdTsdT | 816 |
| XD-09100 | cucccAuuGuGGGcucAcudTsdT | 679 | AGUGAGCCcAcAAUGGGAGdTsdT | 817 |
| XD-09101 | cccAuuGuGGGcucAcucudTsdT | 680 | AGAGUGAGCCcAcAAUGGGdTsdT | 818 |
| XD-09102 | cacucuGcuGcAAauGAGudTsdT | 681 | ACUcAUUUGcAGcAGAGUGdTsdT | 819 |
| XD-09103 | acucuGcuGcAAAuGAGuGdTsdT | 682 | cACUcAUUUGcAGcAGAGUdTsdT | 820 |
| XD-09104 | aguGGGucuuuGGgAAuGcdTsdT | 683 | GcAUUCCcAAAGACCcACUdTsdT | 821 |
| XD-09105 | guGGGucuuuGGGaAuGcAdTsdT | 684 | UGcAUUCCcAAAGACCcACdTsdT | 822 |
| XD-09106 | ggGucuuuGGGAAuGcAAudTsdT | 685 | AUUGcAUUCCcAAAGACCCdTsdT | 823 |
| XD-09107 | uuGGGAAuGcAAuguGcAAdTsdT | 686 | UUGcAcAUUGcAUUCCcAAdTsdT | 824 |
| XD-09108 | guGcAAAuuAuucacAGGGdTsdT | 687 | CCCUGUGAAuAAUUUGcACdTsdT | 825 |
| XD-09109 | cacAGGGcuGuAucAcAucdTsdT | 688 | GAUGUGAuAcAGCCCUGUGdTsdT | 820 |
| XD-09110 | acAGGGcuGuAucacAucGdTsdT | 689 | CGAUGUGAuAcAGCCCUGUdTsdT | 827 |
| XD-09111 | caGGGcuGuAucAcAucGGdTsdT | 690 | CCGAUGUGAuAcAGCCCUGdTsdT | 828 |
| XD-09112 | agGGcuGuAucAcaucGGudTsdT | 691 | ACCGAUGUGAuAcAGCCCUdTsdT | 829 |
| XD-09113 | ggcuGuAucAcAucGGuuAdTsdT | 692 | uAACCGAUGUGAuAcAGCCdTsdT | 830 |
| XD-09114 | gcuGuAucAcAucgGuuAudTsdT | 693 | AuAACCGAUGUGAuAcAGCdTsdT | 831 |
| XD-09115 | cuGuAucAcAucGguuAuudTsdT | 694 | AAuAACCGAUGUGAuAcAGdTsdT | 832 |
| XD-09116 | uguAucAcAucGGuuAuuudTsdT | 695 | AAAuAACCGAUGUGAuAcAdTsdT | 833 |
| XD-09117 | guAucAcAucGGuuAuuuudTsdT | 690 | AAAAuAACCGAUGUGAuACdTsdT | 834 |
| XD-09118 | uaucAcAucGGuuauuuuGdTsdT | 697 | cAAAAuAACCGAUGUGAuAdTsdT | 835 |
| XD-09119 | ucAcAucGGuuAuuuuGGcdTsdT | 698 | GCcAAAAuAACCGAUGUGAdTsdT | 836 |
| XD-09120 | acAucGGuuAuuuuGGcGGdTsdT | 699 | CCGCcAAAAuAACCGAUGUdTsdT | 837 |
| XD-09121 | caucGGuuAuuuugGcGGAdTsdT | 700 | UCCGCcAAAAuAACCGAUGdTsdT | 838 |
| XD-09122 | aucGGuuAuuuuGgcGGAAdTsdT | 701 | UUCCGCcAAAAuAACCGAUdTsdT | 839 |
| XD-09123 | ucGGuuAuuuuGGcGGAAudTsdT | 702 | AUUCCGCcAAAAuAACCGAdTsdT | 840 |
| XD-09124 | cgGuuAuuuuGGcgGAAucdTsdT | 703 | GAUUCCGCcAAAAuAACCGdTsdT | 841 |
| XD-09125 | guuAuuuuGGcGGaAucuudTsdT | 704 | AAGAUUCCGCcAAAAuAACdTsdT | 842 |
| XD-09126 | uuAuuuuGGcGGAaucuucdTsdT | 705 | GAAGAUUCCGCcAAAAuAAdTsdT | 843 |
| XD-09127 | uauuuuGGcGGAAucuucudTsdT | 700 | AGAAGAUUCCGCcAAAAuAdTsdT | 844 |
| XD-09128 | auuuuGGcGGAAucuucuudTsdT | 707 | AAGAAGAUUCCGCcAAAAUdTsdT | 845 |
| XD-09129 | uuuuGGcGGAAucuucuucdTsdT | 708 | GAAGAAGAUUCCGCcAAAAdTsdT | 846 |
| XD-09130 | uuuGGcGGAAucuucuucAdTsdT | 709 | UGAAGAAGAUUCCGCcAAAdTsdT | 847 |
| XD-09131 | aaGuGuGAucAccuGGuuGdTsdT | 710 | cAACcAGGUGAUcAcACUUdTsdT | 848 |
| XD-09132 | uguGAucAccuGGuuGGuGdTsdT | 711 | cACcAACcAGGUGAUcAcAdTsdT | 849 |
| XD-09133 | guGAucAccuGGuuGGuGGdTsdT | 712 | CcACcAACcAGGUGAUcACdTsdT | 850 |
| XD-09134 | ugAucAccuGGuugGuGGcdTsdT | 713 | GCcACcAACcAGGUGAUcAdTsdT | 851 |
| XD-09135 | ucAccuGGuuGGugGcuGudTsdT | 714 | AcAGCcACcAACcAGGUGAdTsdT | 852 |
| XD-09136 | caccuGGuuGGuGgcuGuGdTsdT | 715 | cAcAGCcACcAACcAGGUGdTsdT | 853 |
| XD-09137 | agGAAucAucuuuacuAAAdTsdT | 716 | UUuAGuAAAGAUGAUUCCUdTsdT | 854 |
| XD-09138 | ucAucuuuAcuAAauGccAdTsdT | 717 | UGGcAUUuAGuAAAGAUGAdTsdT | 855 |
| XD-09139 | gaAGAuucuGuuuauGucudTsdT | 718 | AGAcAuAAAcAGAAUCUUCdTsdT | 856 |
| XD-09140 | cuGuGGcccuuAuuuuccAdTsdT | 719 | UGGAAAAuAAGGGCcAcAGdTsdT | 857 |
| XD-09141 | ugGcccuuAuuuuccAcGAdTsdT | 720 | UCGUGGAAAAuAAGGGCcAdTsdT | 858 |
| XD-09142 | ggcccuuAuuuuccAcGAGdTsdT | 721 | CUCGUGGAAAAuAAGGGCCdTsdT | 859 |
| XD-09143 | gcccuuAuuuuccacGAGGdTsdT | 722 | CCUCGUGGAAAAuAAGGGCdTsdT | 860 |
| XD-09144 | cuuAuuuuccAcGaGGAuGdTsdT | 723 | cAUCCUCGUGGAAAAuAAGdTsdT | 861 |
| XD-09145 | uuAuuuuccAcGAgGAuGGdTsdT | 724 | CcAUCCUCGUGGAAAAuAAdTsdT | 862 |
| XD-09146 | uauuuuccAcGAGgAuGGAdTsdT | 725 | UCcAUCCUCGUGGAAAAuAdTsdT | 863 |
| XD-09147 | uuuccAcGAGGAugGAAuAdTsdT | 726 | uAUUCcAUCCUCGUGGAAAdTsdT | 864 |
| XD-09148 | uuccAcGAGGAuGgAAuAAdTsdT | 727 | UuAUUCcAUCCUCGUGGAAdTsdT | 865 |
| XD-09149 | uccAcGAGGAuGGaAuAAudTsdT | 728 | AUuAUUCcAUCCUCGUGGAdTsdT | 866 |
| XD-09150 | cacGAGGAuGGAAuAAuuudTsdT | 729 | AAAUuAUUCcAUCCUCGUGdTsdT | 867 |
| XD-09151 | aauuuccAcAcAAuAAuGAdTsdT | 730 | UcAUuAUUGUGUGGAAAUUdTsdT | 868 |
| XD-09152 | uuuccAcAcAAuAauGAGGdTsdT | 731 | CCUcAUuAUUGUGUGGAAAdTsdT | 869 |
| XD-09153 | cacAcAAuAAuGAgGAAcAdTsdT | 732 | UGUUCCUcAUuAUUGUGUGdTsdT | 870 |
| XD-09154 | uaAuGAGGAAcAuuuuGGGdTsdT | 733 | CCcAAAAUGUUCCUcAUuAdTsdT | 871 |
| XD-09155 | aacAuuuuGGGGcuGGuccdTsdT | 734 | GGACcAGCCCcAAAAUGUUdTsdT | 872 |
| XD-09156 | gccGcuGcucAucauGGucdTsdT | 735 | GACcAUGAUGAGcAGCGGCdTsdT | 873 |
| XD-09157 | aauucuucGGccugAGuAAdTsdT | 736 | UuACUcAGGCCGAAGAAUUdTsdT | 874 |
| XD-09158 | auucuucGGccuGaGuAAcdTsdT | 737 | GUuACUcAGGCCGAAGAAUdTsdT | 875 |
| XD-09159 | uucuucGGccuGAguAAcudTsdT | 738 | AGUuACUcAGGCCGAAGAAdTsdT | 876 |
| XD-09160 | uucGGccuGAGuAacuGuGdTsdT | 739 | cAcAGUuACUcAGGCCGAAdTsdT | 877 |
| XD-09161 | ugAGuAAcuGuGAaAGcAcdTsdT | 740 | GUGCUUUcAcAGUuACUcAdTsdT | 878 |
| XD-09162 | caccAGucAAcuGgAccAAdTsdT | 741 | UUGGUCcAGUUGACUGGUGdTsdT | 879 |
| XD-09163 | caAcuGGAccAAGccAcGcdTsdT | 742 | GCGUGGCUUGGUCcAGUUGdTsdT | 880 |
| XD-09164 | aacuGGAccAAGccAcGcAdTsdT | 743 | UGCGUGGCUUGGUCcAGUUdTsdT | 881 |

TABLE 13
CCR2 mRNA expression level after siRNA treatment

| | Remaining CCR2 | | Remaining | |
|---|---|---|---|---|
| SiRNA | mRNA | | CCR2 mRNA | |
| Duplex ID | (20 nM) | SD | (0.2 nM) | SD |
| XD-09127 | 0.133 | 0.012 | 0.988 | 0.067 |
| XD-09117 | 0.150 | 0.057 | 0.990 | 0.136 |
| XD-09098 | 0.151 | 0.031 | 0.715 | 0.114 |
| XD-09138 | 0.152 | 0.022 | 1.039 | 0.046 |
| XD-09121 | 0.156 | 0.052 | 1.087 | 0.272 |
| XD-09062 | 0.160 | 0.019 | 1.487 | 0.132 |
| XD-09113 | 0.178 | 0.024 | 0.929 | 0.104 |
| XD-09050 | 0.198 | 0.011 | 1.271 | 0.081 |
| XD-09048 | 0.202 | 0.009 | 1.230 | 0.069 |
| XD-09043 | 0.202 | 0.011 | 1.200 | 0.286 |
| XD-09094 | 0.203 | 0.027 | 0.902 | 0.162 |
| XD-09086 | 0.204 | 0.006 | 0.952 | 0.056 |
| XD-09060 | 0.245 | 0.046 | 1.295 | 0.176 |
| XD-09143 | 0.257 | 0.028 | 1.047 | 0.074 |
| XD-09095 | 0.258 | 0.034 | 0.843 | 0.158 |
| XD-09107 | 0.267 | 0.024 | 0.951 | 0.076 |
| XD-09045 | 0.278 | 0.039 | 1.042 | 0.115 |
| XD-09149 | 0.281 | 0.031 | 1.055 | 0.031 |
| XD-09115 | 0.283 | 0.012 | 1.028 | 0.076 |
| XD-09112 | 0.289 | 0.067 | 0.964 | 0.138 |
| XD-09147 | 0.289 | 0.045 | 1.001 | 0.073 |
| XD-09154 | 0.291 | 0.049 | 0.472 | 0.060 |
| XD-09065 | 0.306 | 0.015 | 1.084 | 0.168 |
| XD-09146 | 0.311 | 0.028 | 0.979 | 0.065 |
| XD-09123 | 0.315 | 0.121 | 1.201 | 0.312 |
| XD-09047 | 0.320 | 0.035 | 1.091 | 0.147 |
| XD-09125 | 0.332 | 0.117 | 0.750 | 0.034 |
| XD-09109 | 0.367 | 0.037 | 0.999 | 0.106 |
| XD-09046 | 0.370 | 0.072 | 1.011 | 0.103 |
| XD-09044 | 0.371 | 0.018 | 1.187 | 0.185 |
| XD-09053 | 0.375 | 0.071 | 1.384 | 0.152 |
| XD-09088 | 0.379 | 0.030 | 0.923 | 0.076 |
| XD-09139 | 0.404 | 0.044 | 0.980 | 0.073 |
| XD-09137 | 0.425 | 0.051 | 1.031 | 0.052 |
| XD-09041 | 0.443 | 0.036 | 1.159 | 0.233 |
| XD-09055 | 0.459 | 0.123 | 1.159 | 0.145 |
| XD-09051 | 0.476 | 0.049 | 1.380 | 0.108 |
| XD-09158 | 0.483 | 0.029 | 0.930 | 0.116 |
| XD-09027 | 0.486 | 0.048 | 0.980 | 0.102 |
| XD-09090 | 0.490 | 0.012 | 0.953 | 0.171 |
| XD-09059 | 0.494 | 0.063 | 1.200 | 0.186 |
| XD-09052 | 0.497 | 0.046 | 1.392 | 0.020 |
| XD-09054 | 0.503 | 0.071 | 1.078 | 0.116 |
| XD-09039 | 0.512 | 0.045 | 1.200 | 0.180 |
| XD-09161 | 0.514 | 0.045 | 0.816 | 0.214 |
| XD-09159 | 0.522 | 0.049 | 0.762 | 0.134 |
| XD-09153 | 0.541 | 0.078 | 0.745 | 0.085 |
| XD-09130 | 0.548 | 0.024 | 0.770 | 0.052 |
| XD-09141 | 0.550 | 0.064 | 0.964 | 0.097 |
| XD-09135 | 0.551 | 0.087 | 0.980 | 0.104 |
| XD-09049 | 0.552 | 0.055 | 1.320 | 0.160 |
| XD-09096 | 0.554 | 0.055 | 0.776 | 0.084 |
| XD-09157 | 0.556 | 0.063 | 0.807 | 0.197 |
| XD-09036 | 0.557 | 0.024 | 1.067 | 0.155 |
| XD-09114 | 0.558 | 0.076 | 0.939 | 0.129 |
| XD-09030 | 0.559 | 0.034 | 1.113 | 0.172 |
| XD-09035 | 0.567 | 0.038 | 1.127 | 0.177 |
| XD-09108 | 0.569 | 0.093 | 1.090 | 0.147 |
| XD-09152 | 0.576 | 0.077 | 1.020 | 0.072 |
| XD-09101 | 0.609 | 0.050 | 0.945 | 0.029 |
| XD-09118 | 0.615 | 0.262 | 1.081 | 0.285 |
| XD-09061 | 0.620 | 0.060 | 1.432 | 0.132 |
| XD-09140 | 0.626 | 0.077 | 0.951 | 0.085 |
| XD-09126 | 0.628 | 0.082 | 0.965 | 0.104 |
| XD-09124 | 0.631 | 0.229 | 0.914 | 0.177 |
| XD-09028 | 0.631 | 0.041 | 1.100 | 0.123 |
| XD-09156 | 0.638 | 0.086 | 0.814 | 0.129 |
| XD-09129 | 0.657 | 0.039 | 0.930 | 0.137 |
| XD-09150 | 0.658 | 0.118 | 1.020 | 0.072 |
| XD-09080 | 0.672 | 0.025 | 0.795 | 0.082 |
| XD-09082 | 0.672 | 0.076 | 0.893 | 0.073 |
| XD-09122 | 0.673 | 0.221 | 1.062 | 0.288 |
| XD-09042 | 0.697 | 0.060 | 1.228 | 0.279 |
| XD-09075 | 0.705 | 0.042 | 1.022 | 0.040 |
| XD-09164 | 0.713 | 0.110 | 0.659 | 0.116 |
| XD-09128 | 0.714 | 0.081 | 0.924 | 0.035 |
| XD-09120 | 0.716 | 0.239 | 1.000 | 0.210 |
| XD-09163 | 0.721 | 0.023 | 0.756 | 0.112 |
| XD-09058 | 0.725 | 0.096 | 1.190 | 0.251 |
| XD-09162 | 0.737 | 0.038 | 0.802 | 0.111 |
| XD-09116 | 0.738 | 0.021 | 0.969 | 0.134 |
| XD-09151 | 0.739 | 0.056 | 1.006 | 0.105 |
| XD-09099 | 0.742 | 0.070 | 1.021 | 0.046 |
| XD-09105 | 0.751 | 0.079 | 0.895 | 0.043 |
| XD-09056 | 0.756 | 0.232 | 1.203 | 0.091 |
| XD-09089 | 0.765 | 0.057 | 0.810 | 0.078 |
| XD-09110 | 0.768 | 0.039 | 1.071 | 0.034 |
| XD-09111 | 0.771 | 0.096 | 1.007 | 0.067 |
| XD-09155 | 0.772 | 0.151 | 1.077 | 0.267 |
| XD-09160 | 0.781 | 0.059 | 0.983 | 0.131 |
| XD-09057 | 0.785 | 0.158 | 1.161 | 0.144 |
| XD-09142 | 0.787 | 0.067 | 0.968 | 0.039 |
| XD-09145 | 0.792 | 0.123 | 0.878 | 0.135 |
| XD-09091 | 0.793 | 0.156 | 0.894 | 0.110 |
| XD-09144 | 0.797 | 0.115 | 0.958 | 0.078 |
| XD-09119 | 0.801 | 0.269 | 1.095 | 0.292 |
| XD-09066 | 0.801 | 0.053 | 0.941 | 0.146 |
| XD-09148 | 0.804 | 0.112 | 1.061 | 0.102 |
| XD-09081 | 0.830 | 0.076 | 0.893 | 0.060 |
| XD-09063 | 0.831 | 0.105 | 1.172 | 0.413 |
| XD-09072 | 0.841 | 0.050 | 0.982 | 0.123 |
| XD-09033 | 0.843 | 0.045 | 1.072 | 0.194 |
| XD-09106 | 0.850 | 0.100 | 0.940 | 0.101 |
| XD-09136 | 0.855 | 0.079 | 0.937 | 0.113 |
| XD-09087 | 0.858 | 0.103 | 0.878 | 0.109 |
| XD-09038 | 0.860 | 0.020 | 1.118 | 0.213 |
| XD-09131 | 0.862 | 0.086 | 0.791 | 0.172 |
| XD-09037 | 0.865 | 0.071 | 1.062 | 0.218 |
| XD-09134 | 0.869 | 0.118 | 0.762 | 0.140 |
| XD-09040 | 0.883 | 0.043 | 1.275 | 0.273 |
| XD-09100 | 0.888 | 0.117 | 0.930 | 0.068 |
| XD-09079 | 0.890 | 0.040 | 0.765 | 0.136 |
| XD-09102 | 0.901 | 0.144 | 0.937 | 0.123 |
| XD-09032 | 0.904 | 0.057 | 1.036 | 0.098 |
| XD-09031 | 0.905 | 0.048 | 1.152 | 0.167 |
| XD-09132 | 0.911 | 0.064 | 0.818 | 0.056 |
| XD-09076 | 0.917 | 0.028 | 1.040 | 0.198 |
| XD-09092 | 0.925 | 0.093 | 0.968 | 0.166 |
| XD-09074 | 0.937 | 0.046 | 0.941 | 0.172 |
| XD-09085 | 0.939 | 0.017 | 0.925 | 0.047 |
| XD-09083 | 0.954 | 0.098 | 0.938 | 0.043 |
| XD-09078 | 0.962 | 0.043 | 0.919 | 0.144 |
| XD-09073 | 0.963 | 0.045 | 0.905 | 0.101 |
| XD-09071 | 0.969 | 0.085 | 0.896 | 0.195 |
| XD-09029 | 0.972 | 0.070 | 1.051 | 0.101 |
| XD-09084 | 0.973 | 0.069 | 0.869 | 0.059 |
| XD-09064 | 0.984 | 0.054 | 1.060 | 0.120 |
| XD-09133 | 0.987 | 0.030 | 0.951 | 0.170 |
| XD-09103 | 1.003 | 0.092 | 0.946 | 0.105 |
| XD-09097 | 1.010 | 0.140 | 0.727 | 0.128 |
| XD-09104 | 1.014 | 0.109 | 0.877 | 0.072 |
| XD-09034 | 1.031 | 0.117 | 1.186 | 0.116 |
| XD-09093 | 1.046 | 0.130 | 0.810 | 0.134 |
| XD-09067 | 1.058 | 0.147 | 1.016 | 0.127 |
| XD-09069 | 1.081 | 0.018 | 0.886 | 0.149 |
| XD-09070 | 1.089 | 0.064 | 0.860 | 0.160 |
| XD-09068 | 1.102 | 0.054 | 0.918 | 0.138 |
| XD-09077 | 1.153 | 0.121 | 0.943 | 0.112 |
| XD-00379 | 0.851 | 0.034 | 1.020 | 0.207 |
| (Negative Control) | | | | |
| XD- | 0.891 | 0.095 | 0.863 | 0.068 |
| 00385(Negative | | | | |
| control) | | | | |
| XD-00033 (Aha-1; | 0.186 | 0.044 | 0.678 | 0.067 |
| positive control) | | | | |

Dose Response of Selected CCR2 siRNA Duplexes

CCR2 siRNA duplexes that caused a significant reduction of CCR2 mRNA level in the dual dose screening were selected and further tested for the dose response. Human monocytic THP-1 cells were cultured and maintained in 96-well plates at a density of 25,000 cells per well. THP-1 monocytes were transfected with CCR2 siRNA duplexes selected from the previous dual dose screening at various concentrations using Lipofectomine® 2000 (0.5 μl/well). The doses for each CCR2 siRNA duplex included 50.0 nM, 10.0 nM, 2.0 nM, 0.4 nM, 0.8×10⁻¹ nM, 1.6×10⁻² nM, 3.2×10⁻³ nM, 6.4×10⁻⁴ nM, 1.28×10⁻⁴ nM, and 2.6×10⁻⁵ nM. Following incubation of 24 hours, the treated cells were harvested and the remaining CCR2 mRNA level was measured in each condition. The IC₅₀ value for each CCR2 siRNA duplex was determined as shown in Table 14 and each dose response is shown in FIG. 1B.

TABLE 14
IC₅₀ of CCR2 siRNA duplexes

| siRNA Duplex ID | IC₅₀ (nM) |
|---|---|
| XD-09048 | 0.17046435 |
| XD-09050 | 0.11069267 |
| XD-09062 | 0.38493077 |
| XD-09086 | 0.2956292 |
| XD-09094 | 0.27921953 |
| XD-09098 | 0.01619127 |
| XD-09113 | 0.70462092 |
| XD-09117 | 0.13414519 |
| XD-09121 | 0.20361795 |
| XD-09127 | 0.15636242 |
| XD-09138 | 0.23593031 |
| XD-09154 | 3.37845447 |

### Example 5: Dual Dose Screening of CSF1R siRNA Duplexes and Variants

siRNA modification variants derived from siRNA duplexes with high efficiency and specificity to CSF1R mRNA knock-down were designed by sequence and chemical modifications and the resulting duplex variants were further tested in Hepa 1-6 cells. Hepa 1-6 cells derived from mouse hepatoma were cultured with the standard culture condition and maintained in 96-well plates at a density of 15,000 cells per well. Hepa 1-6 cells were transfected with CSF1R siRNA duplexes and variants using Lipofectomine® 2000 (0.5 μl/well). A total of 59 CSF1R siRNA duplexes including variants from the original modified siRNA duplexes were introduced into Hepa 1-6 cells and further validated (Table 15). The CSF1R siRNAs were transfected at a final concentration of 0.2 nM and 20 nM, respectively. An anti R-Luc siRNA duplex (XD-00379) and a scramble RNA duplex (XD-00194) were used as positive and negative control, respectively. The information and sequences of these siRNA duplexes are shown in Table 15.

TABLE 15
CSF1R siRNA duplexes and variants

| | | | | SEQ | | | SEQ |
|---|---|---|---|---|---|---|---|
| | Duplex- | SS | | ID | AS | | ID |
| Position | ID | ID | Sense Sequence | NO | ID | Antisense Sequence | NO |
| 948 | XD- | X28782 | cuGcccAGAucGugu | 382 | X28783 | GAGcAcACGAUCUG | 501 |
| | 08917 | | GcucdTsdT | | | GGcAGdTsdT | |
| 1056 | XD- | X28792 | accGuuAccAAAAa | 387 | X28793 | AGGACUUUUUGGu | 506 |
| | 08922 | | GuccudTsdT | | | AACGGUdTsdT | |
| 1060 | XD- | X28794 | uuAccAAAAAGucc | 388 | X28795 | GGUcAGGACUUUU | 507 |
| | 08923 | | uGAccdTsdT | | | UGGuAAdTsdT | |
| 1717 | XD- | X28820 | gaccuuAGAGcAca | 401 | X28821 | UUGGUUGUGCUCu | 520 |
| | 08936 | | AccAAdTsdT | | | AAGGUCdTsdT | |
| 3049 | XD- | X28836 | uccGcuGGAAGAuc | 409 | X28837 | UCGAUGAUCUUCcA | 528 |
| | 08944 | | AucGAdTsdT | | | GCGGAdTsdT | |
| 1935 | XD- | X28842 | caucGAGAGcuAug | 431 | X28843 | GCCCUcAuAGCUCU | 531 |
| | 08947 | | AGGGcdTsdT | | | CGAUGdTsdT | |
| 2276 | XD- | X28872 | aucAcGGAGuAcug | 428 | X28873 | AGcAAcAGuACUCC | 547 |
| | 08963 | | uuGcudTsdT | | | GUGAUdTsdT | |
| 2295 | XD- | X28884 | auGGcGAccuGcuc | 434 | X28885 | AAGUUGAGcAGGU | 553 |
| | 08969 | | AAcuudTsdT | | | CGCcAUdTsdT | |
| 2600 | XD- | X28896 | gcuuccAAGAAuug | 440 | X28897 | GGAUGcAAUUCUU | 559 |
| | 08975 | | cAuccdTsdT | | | GGAAGCdTsdT | |
| 2781 | XD- | X28910 | guGucuAcAcGGuu | 447 | X28911 | CUCUGAACCGUGuA | 566 |
| | 08982 | | cAGAGdTsdT | | | GAcACdTsdT | |
| 2791 | XD- | X28916 | gguucAGAGcGAcg | 450 | X28917 | CcAGACGUCGCUCU | 569 |
| | 08985 | | ucuGGdTsdT | | | GAACCdTsdT | |
| 2793 | XD- | X28918 | uucAGAGcGAcGuc | 451 | X28919 | GACcAGACGUCGCU | 570 |
| | 08986 | | uGGucdTsdT | | | CUGAAdTsdT | |
| 2874 | XD- | X28922 | ugAAcAGcAAGuu | 453 | X28923 | UuAuAGAACUUGCU | 572 |
| | 08988 | | cuAuAAdTsdT | | | GUUcAdTsdT | |
| 2880 | XD- | X28924 | gcAAGuucuAuAAa | 454 | X28925 | ACcAGUUuAuAGAA | 573 |
| | 08989 | | cuGGudTsdT | | | CUUGCdTsdT | |
| 1948 | XD- | X28932 | agAGcGGGAcuAua | 458 | X28933 | AUUGGuAuAGUCCC | 577 |
| | 08993 | | ccAAudTsdT | | | GCUCUdTsdT | |
| 3292 | XD- | X28952 | acAAAcucuGccuuc | 468 | X28953 | GACCGAAGGcAGA | 587 |
| | 09003 | | GGucdTsdT | | | GUUUGUdTsdT | |
| 3305 | XD- | X28958 | ucGGucAuuucAcuc | 471 | X28959 | UGUUGAGUGAAAU | 590 |
| | 09006 | | AAcAdTsdT | | | GACCGAdTsdT | |
| 3916 | XD- | X28976 | cuGAcucAuccuAac | 480 | X28977 | GUuAGUuAGGAUGA | 599 |
| | 09015 | | uAAcdTsdT | | | GUcAGdTsdT | |
| 3917 | XD- | X28978 | ugAcucAuccuAAcu | 481 | X28979 | UGUuAGUuAGGAUG | 600 |
| | 09016 | | AAcAdTsdT | | | AGUcAdTsdT | |
| 3959 | XD- | X28988 | ccAcAuuAAAcuAa | 486 | X28989 | UGCUGUuAGUUuAA | 605 |
| | 09021 | | cAGcAdTsdT | | | UGUGGdTsdT | |
| 1935_ | XD- | X32298 | ucCGcuGGAaGAuc | 883 | X32372 | dTCGAUGAUCUUCc | 922 |
| var1 | 10343 | | AuCgsa | | | AGCGGAusu | |
| 1948_ | XD- | X32299 | caUcGAGAGcuAug | 884 | X32373 | dTCCCUcAuAGCUcU | 923 |
| var1 | 10344 | | AGGgsa | | | CGAUGusu | |
| 2874_ | XD- | X32300 | ugAAcAGCAaGUu | 885 | X32374 | dTUAuAGAACUUGc | 924 |
| var1 | 10345 | | cuAUasa | | | UGUUcAusu | |
| 3049_ | XD- | X32301 | agAGcGGGAcuAua | 886 | X32375 | dTUUGGuAuAGUCcC | 925 |
| var1 | 10346 | | ccAasa | | | GCUCUusu | |
| 3917_ | XD- | X32302 | ugAcucAUCcuAAc | 887 | X32376 | dTGUuAGUuAGGAu | 926 |
| var1 | 10347 | | uAAcsa | | | GAGUcAusu | |
| 1935_ | XD- | X32303 | uccGcuGGAaGAuc | 888 | X32372 | dTCGAUGAUCUUCc | 927 |
| var2 | 10348 | | Aucgsa | | | AGCGGAusu | |
| 1948_ | XD- | X32304 | caucGAGAGcuAug | 889 | X32373 | dTCCCUcAuAGCUcU | 928 |
| var2 | 10349 | | AGGgsa | | | CGAUGusu | |
| 2874_ | XD- | X32305 | ugAAcAGcAaGuuc | 890 | X32374 | dTUAuAGAACUUGc | 929 |
| var2 | 10350 | | uAuasa | | | UGUUcAusu | |
| 3049_ | XD- | X32301 | agAGcGGGAcuAua | 891 | X32377 | dTUUGGuAuAGUCcC | 930 |
| var2 | 10351 | | ccAasa | | | gCUCUusu | |
| 3917_ | XD- | X32306 | ugAcucAuccuAAcu | 892 | X32376 | dTGUuAGUuAGGAu | 931 |
| var2 | 10352 | | AAcsa | | | GAGUcAusu | |
| 1935_ | XD- | X32307 | ucCGcuGGAaGAuc | 893 | X32378 | UCGAUGAUCUUCcA | 932 |
| var3 | 10353 | | AuCga(C6) | | | GCGGAusu | |
| 1948_ | XD- | X32308 | caUcGAGAGcuAug | 894 | X32379 | UCCCUcAuAGCUcU | 933 |
| var3 | 10354 | | AGGga(C6) | | | CGAUGusu | |
| 2874_ | XD- | X32309 | ugAAcAGCAaGUu | 895 | X32380 | UUAuAGAACUUGcU | 934 |
| var3 | 10355 | | cuAUaa(C6) | | | GUUcAusu | |
| 3049_ | XD- | X32310 | agAGcGGGAcuAua | 896 | X32381 | UUUGGuAuAGUCcC | 935 |
| var3 | 10356 | | ccAaa(C6) | | | GCUCUusu | |
| 3917_ | XD- | X32311 | ugAcucAUCcuAAc | 897 | X32382 | UGUuAGUuAGGAuG | 936 |
| var3 | 10357 | | uAAca(C6) | | | AGUcAusu | |
| 948_ | XD- | X32312 | cuGcccAGAucGugu | 898 | X32383 | dTAGcAcACGAUCuG | 937 |
| var1 | 10358 | | GCusa | | | GGcAGusu | |
| 1056_ | XD- | X32313 | acCGuuACCaAAA | 899 | X32384 | dTGGACUUUUUGGu | 938 |
| var1 | 10359 | | aGuCcsa | | | AACGGUusu | |
| 1060_ | XD- | X32314 | uuAccAAAAaGUcc | 900 | X32385 | dTGUcAGGACUUUu | 939 |
| var1 | 10360 | | uGAcsa | | | UGGuAAusu | |
| 1717_ | XD- | X32315 | gaCcuuAGAgcAca | 901 | X32386 | dTUGGUUGUGCUCu | 940 |
| var1 | 10361 | | AcCasa | | | AAGGUCusu | |
| 2276_ | XD- | X32316 | auCAcGGAGuACu | 902 | X32387 | dTGcAAcAGuACUcC | 941 |
| var1 | 10362 | | guuGcsa | | | GUGAUusu | |
| 2295_ | XD- | X32317 | auGGcGACCuGCu | 903 | X32388 | dTAGUUGAGcAGGu | 942 |
| var1 | 10363 | | cAACusa | | | CGCcAUusu | |
| 2600_ | XD- | X32318 | gcUuccAAGaAUug | 904 | X32389 | dTGAUGcAAUUCUu | 943 |
| var1 | 10364 | | cAUcsa | | | GGAAGCusu | |
| 2781_ | XD- | X32319 | guGucuACAcGGuu | 905 | X32390 | dTUCUGAACCGUGu | 944 |
| var1 | 10365 | | cAGasa | | | AGAcACusu | |
| 2791_ | XD- | X32320 | ggUucAGAGcGAc | 906 | X32391 | dTCAGACGUCGCUc | 945 |
| var1 | 10366 | | gucUgsa | | | UGAACCusu | |
| 2793_ | XD- | X32321 | uuCAGAGCGacGu | 907 | X32392 | dTACcAGACGUCGc | 946 |
| var1 | 10367 | | cuGGusa | | | UCUGAAusu | |
| 2880_ | XD- | X32322 | gcAAGuUCUauAA | 908 | X32393 | dTCcAGUUuAuAGaA | 947 |
| var1 | 10368 | | acuGgsa | | | CUUGCusu | |
| 3292_ | XD- | X32323 | acAAAcUCUgcCuu | 909 | X32394 | dTACCGAAGGcAGa | 948 |
| var1 | 10369 | | cGGusa | | | GUUUGUusu | |
| 3305_ | XD- | X32324 | ucGGucAUUucAcu | 910 | X32395 | dTGUUGAGUGAAAu | 949 |
| var1 | 10370 | | cAAcsa | | | GACCGAusu | |
| 3916_ | XD- | X32325 | cuGAcuCAUccUAa | 911 | X32396 | dTUuAGUuAGGAUg | 950 |
| var1 | 10371 | | cuAasa | | | AGUcAGusu | |
| 3959_ | XD- | X32326 | ccAcAuUAAacUAa | 912 | X32397 | dTGCUGUuAGUUua | 951 |
| var1 | 10372 | | cAGcsa | | | AUGUGGusu | |
| 973_ | XD- | X32327 | caGcGuUGAuGUua | 913 | X32398 | dTAAGUuAAcAUcaA | 952 |
| var1 | 10373 | | AcUusa | | | CGCUGusu | |
| 1087_ | XD- | X32328 | cgAucAAGUaGAu | 914 | X32399 | dTUGGAAAUCuACu | 953 |
| var1 | 10374 | | uucCasa | | | UGAUCGusu | |
| 1941_ | XD- | X32329 | ggAAGAUCAucG | 915 | X32400 | dTAGCUCUCGAUGa | 954 |
| var1 | 10375 | | AgAGCusa | | | UCUUCCusu | |
| 1964_ | XD- | X32330 | ggCAAcAGUuAU | 916 | X32401 | dTGAAAGuAuAACu | 955 |
| var1 | 10376 | | AcuuUcsa | | | GUUGCCusu | |
| 2689_ | XD- | X32331 | ggCuAGGGAcAUc | 917 | X32402 | dTUUcAUGAUGUCc | 956 |
| var1 | 10377 | | auGAasa | | | CuAGCCusu | |
| 2896_ | XD- | X32332 | ggUGAAGGAuGG | 918 | X32403 | dTUGGuAUCcAUCcU | 957 |
| var1 | 10378 | | AuAcCasa | | | UcACCusu | |
| 3527_ | XD- | X32333 | ggAAAuGGAcuGA | 919 | X32404 | dTUAAAGUcAGUCc | 958 |
| var1 | 10379 | | cuuUasa | | | AUUUCCusu | |
| 3911_ | XD- | X32334 | ccAAGcUGAcuCA | 920 | X32405 | dTUAGGAUGAGUca | 959 |
| var1 | 10380 | | uccUasa | | | GCUUGGusu | |
| 3962_ | XD- | X32335 | caUuAAACUaACA | 921 | X32406 | dTAAUGCUGUuAGu | 960 |
| var1 | 10381 | | gcAUusa | | | UuAAUGusu | |

After incubating for 24 hours, the treated cells were harvested and the remaining CSF1R mRNA level was measured in each condition and ranked. The remaining CSF1R mRNA level after the 24 hours treatment with each siRNA duplex is listed in Table 16. Among the 59 duplexes tested, 5 siRNAs and the variants thereof that have the most reduced CSF1R mRNA level were selected, including duplex XD-08944 and its variants, XD-10343, XD-10348 and XD-10353; duplex XD-08947 and its variants, XD-10344, XD-10349 and XD-10354; duplex XD-08988 and its variants, XD-10345, XD-10350 and XD-10355; duplex XD-08993 and its variants, XD-10346, XD-10351 and XD-10356; and duplex XD-09016 and its variants, XD-10347, XD-10352 and XD-10357. The top 20 siRNAs and their modification variants were also ranked, including XD-08927 and its variant XD-10358; XD-08922 and its variant XD-10359; XD-08923 and its variant XD-10360; XD-08936 and its variant XD-10361; XD-08963 and its variant XD-10362; XD-08969 and its variant XD-10363; XD-08975 and its variant XD-10364; XD-08982 and its variant XD-10365; XD-08985 and its variant XD-10366; XD-08986 and its variant XD-10367; XD-08989 and its variant XD-10368; XD-09003 and its variant XD-10369; XD-09006 and its variant XD-10370; XD-09015 and its variant XD-10371; and XD-09021 and its variant XD-10372 (Table 16). The data also indicate several other siRNA modifications that result in significant reduction of expression, such as XD-10373, XD-10374, XD-10375, XD-10376, XD-10377, XD-10378, XD-10379, XD-10380, and XD-10381.

TABLE 16
CSF1R mRNA expression after siRNA treatment

| | Remaining | | Remaining | |
|---|---|---|---|---|
| Duplex | mRNA | | mRNA | |
| ID | (20 nM siRNA) | SD | (0.2 nM siRNA) | SD |
| XD-08944 | 0.136 | 0.009 | 0.244 | 0.028 |
| XD-10343 | 0.128 | 0.007 | 0.207 | 0.011 |
| XD-10348 | 0.145 | 0.015 | 0.210 | 0.007 |
| XD-10353 | 0.130 | 0.006 | 0.269 | 0.017 |
| XD-08947 | 0.256 | 0.012 | 0.655 | 0.042 |
| XD-10344 | 0.231 | 0.006 | 0.387 | 0.028 |
| XD-10349 | 0.233 | 0.013 | 0.380 | 0.028 |
| XD-10354 | 0.259 | 0.019 | 0.413 | 0.036 |
| XD-08988 | 0.162 | 0.009 | 0.275 | 0.009 |
| XD-10345 | 0.122 | 0.009 | 0.161 | 0.010 |
| XD-10350 | 0.109 | 0.007 | 0.181 | 0.014 |
| XD-10355 | 0.112 | 0.009 | 0.196 | 0.013 |
| XD-08993 | 0.218 | 0.009 | 0.403 | 0.039 |
| XD-10346 | 0.193 | 0.011 | 0.359 | 0.016 |
| XD-10351 | 0.233 | 0.009 | 0.327 | 0.016 |
| XD-10356 | 0.198 | 0.006 | 0.369 | 0.023 |
| XD-09016 | 0.118 | 0.007 | 0.180 | 0.009 |
| XD-10347 | 0.110 | 0.008 | 0.172 | 0.007 |
| XD-10352 | 0.117 | 0.005 | 0.181 | 0.014 |
| XD-10357 | 0.103 | 0.010 | 0.174 | 0.013 |
| XD-08917 | 0.120 | 0.008 | 0.392 | 0.003 |
| XD-10358 | 0.132 | 0.007 | 0.194 | 0.010 |
| XD-08922 | 0.100 | 0.005 | 0.202 | 0.017 |
| XD-10359 | 0.078 | 0.005 | 0.154 | 0.023 |
| XD-08923 | 0.072 | 0.010 | 0.236 | 0.021 |
| XD-10360 | 0.068 | 0.002 | 0.149 | 0.019 |
| XD-08936 | 0.131 | 0.002 | 0.332 | 0.030 |
| XD-10361 | 0.119 | 0.006 | 0.290 | 0.033 |
| XD-08963 | 0.186 | 0.006 | 0.465 | 0.041 |
| XD-10362 | 0.207 | 0.009 | 0.432 | 0.044 |
| XD-08969 | 0.279 | 0.022 | 0.465 | 0.055 |
| XD-10363 | 0.166 | 0.006 | 0.378 | 0.038 |
| XD-08975 | 0.167 | 0.013 | 0.582 | 0.036 |
| XD-10364 | 0.140 | 0.011 | 0.352 | 0.045 |
| XD-08982 | 0.221 | 0.026 | 0.494 | 0.035 |
| XD-10365 | 0.166 | 0.013 | 0.400 | 0.024 |
| XD-08985 | 0.286 | 0.008 | 0.784 | 0.041 |
| XD-10366 | 0.196 | 0.017 | 0.543 | 0.063 |
| XD-08986 | 0.195 | 0.021 | 0.376 | 0.019 |
| XD-10367 | 0.192 | 0.014 | 0.284 | 0.030 |
| XD-08989 | 0.250 | 0.025 | 0.751 | 0.032 |
| XD-10368 | 0.206 | 0.019 | 0.545 | 0.045 |
| XD-09003 | 0.197 | 0.018 | 0.591 | 0.014 |
| XD-10369 | 0.131 | 0.014 | 0.238 | 0.015 |
| XD-09006 | 0.146 | 0.006 | 0.321 | 0.030 |
| XD-10370 | 0.074 | 0.003 | 0.214 | 0.015 |
| XD-09015 | 0.251 | 0.012 | 0.459 | 0.069 |
| XD-10371 | 0.159 | 0.015 | 0.194 | 0.009 |
| XD-09021 | 0.117 | 0.008 | 0.224 | 0.035 |
| XD-10372 | 0.117 | 0.006 | 0.147 | 0.020 |
| XD-10373 | 0.042 | 0.005 | 0.095 | 0.020 |
| XD-10374 | 0.061 | 0.002 | 0.111 | 0.012 |
| XD-10375 | 0.073 | 0.007 | 0.284 | 0.036 |
| XD-10376 | 0.202 | 0.010 | 0.362 | 0.036 |
| XD-10377 | 0.644 | 0.037 | 0.571 | 0.046 |
| XD-10378 | 0.212 | 0.008 | 0.378 | 0.048 |
| XD-10379 | 0.077 | 0.004 | 0.175 | 0.013 |
| XD-10380 | 0.126 | 0.014 | 0.191 | 0.020 |
| XD-10381 | 0.094 | 0.005 | 0.166 | 0.021 |
| XD-00194 | 0.965 | 0.117 | 0.980 | 0.048 |
| (negative | | | | |
| control) | | | | |
| XD-00379 | 0.009 | 0.003 | 0.069 | 0.009 |
| (Renilla | | | | |
| Luciferase | | | | |
| control) | | | | |

Dose Response of Selected CSF1R siRNA Duplexes and Variants

CSF1R siRNA duplexes and variants that caused a significant reduction of CSF1R mRNA level in the dual dose screening were selected and further tested for dose responses. Hepa 1-6 cells were cultured and maintained in 96-well plates at a density of 15,000 cells per well. Hepa 1-6 cells were transfected with CSF1R siRNA duplexes and variants selected from the previous dual dose screening at various concentrations using Lipofectomine® 2000 (0.5 μl/well). The doses for each CSF1R siRNA duplex included 50 nM, 10 nM, 2.0 nM, 0.40 nM, 0.08 nM, 1.6×10⁻² nM, 3.2×10⁻³ nM, 6.4×10⁻⁴ nM, 1.28×10⁻⁴ nM, and 2.6×⁻⁵ nM. Following incubation of 24 hours, the treated cells were harvested and the remaining CSF1R mRNA level was measured in each condition. The IC₅₀ and IC₈₀ values of each CSF1R duplex was determined (shown in Table 17) and each dose response curve is shown in FIG. 1C.

TABLE 17
IC₅₀ and IC₈₀ of CSF1R siRNA duplexes

| siRNA Duplex ID | IC₅₀ (nM) | IC₈₀ (nM) | Maximal KD |
|---|---|---|---|
| XD-10373 | 0.01566477 | 0.10114948 | 95% |
| XD-10381 | 0.02151724 | 0.26504219 | 88% |
| XD-09016 | 0.04774574 | 0.52067604 | 87% |
| XD-10347 | 0.02366698 | 0.25339097 | 87% |
| XD-10357 | 0.03270323 | 0.30401297 | 96% |
| XD-10345 | 0.03142479 | 0.42640625 | 87% |
| XD-10350 | 0.02893749 | 0.29063486 | 86% |
| XD-10359 | 0.03676781 | 0.21262353 | 91% |
| XD-10360 | 0.03227182 | 0.2567045 | 92% |
| XD-10371 | 0.03299214 | 0.4377136 | 84% |
| XD-09021 | 0.04241552 | 0.34612646 | 87% |
| XD-10372 | 0.01809142 | 0.21942896 | 87% |

### Example 6: Dual Response Screening of CCR2 siRNA Duplexes and Variants

siRNA modification variants derived from siRNA duplexes with high efficiency and specificity to CCR2 mRNA knock-down were designed according to sequence and chemical modifications and the resulting duplex variants were further tested in Hepa 1-6 cell. Hepa 1-6 cells were cultured using the standard culture condition and maintained in 96-well plates at a density of 15,000 cells per well. Hepa 1-6 cells were transfected with CCR2 siRNA duplexes using Lipofectomine® 2000 (0.5 μl/well). A total of 61 siRNA duplexes, including variants from the original modified siRNA duplexes, were transfected into Hepa 1-6 cells and further validated (Table 18). The CCR2 siRNAs were transfected at a final concentration of 0.2 nM and 20 nM, respectively. An anti R-Luc siRNA duplex (XD-00379) and a scramble RNA duplex (XD-00194) were used as positive and negative control, respectively. After incubating for 24 hours, the treated cells were harvested and the remaining CCR2 mRNA level was measured and ranked. The information and sequences of these siRNA duplexes are included in Table 18.

TABLE 18
CCR2 siRNA duplexes and variants

| | | | | SEQ | | | SEQ |
|---|---|---|---|---|---|---|---|
| | Duplex- | SS | | ID | AS | | ID |
| Position | ID | ID | Sense Sequence | NO | ID | Antisense Sequence | NO |
| 489 | XD- | X29044 | cuGuccAcAucucguuc | 622 | X29045 | GAGAACGAGAUG | 760 |
| | 09043 | | ucdTsdT | | | UGGAcAGdTsdT | |
| 492 | XD- | X29048 | uccAcAucucGuucuc | 624 | X29049 | ACCGAGAACGAGA | 762 |
| | 09045 | | GGudTsdT | | | UGUGGAdTsdT | |
| 495 | XD- | X29054 | acAucucGuucucgGu | 627 | X29055 | uAAACCGAGAACG | 765 |
| | 09048 | | uuAdTsdT | | | AGAUGUdTsdT | |
| 498 | XD- | X29058 | ucucGuucucGGuuuA | 629 | X29059 | UGAuAAACCGAGA | 767 |
| | 09050 | | ucAdTsdT | | | ACGAGAdTsdT | |
| 533 | XD- | X29078 | cgGuGAAGAAGuca | 639 | X29079 | GGUGGUGACUUC | 777 |
| | 09060 | | ccAccdTsdT | | | UUcACCGdTsdT | |
| 544 | XD- | X29082 | ucAccAccuuuuuuGA | 641 | X29083 | uAAUcAAAAAAGG | 779 |
| | 09062 | | uuAdTsdT | | | UGGUGAdTsdT | |
| 590 | XD- | X29130 | ugAcGuGAAGcAAa | 665 | X29131 | CCcAAUUUGCUUc | 803 |
| | 09086 | | uuGGGdTsdT | | | ACGUcAdTsdT | |
| 693 | XD- | X29146 | ugcAAAAAGcuGAa | 673 | X29147 | AGcACUUcAGCUU | 811 |
| | 09094 | | GuGcudTsdT | | | UUUGcAdTsdT | |
| 698 | XD- | X29148 | aaAGcuGAAGuGcu | 674 | X29149 | AGUcAAGcACUUcA | 812 |
| | 09095 | | uGAcudTsdT | | | GCUUUdTsdT | |
| 751 | XD- | X29154 | ugcuuuuucuuAuuAcu | 677 | X29155 | AGAGuAAuAAGAA | 815 |
| | 09098 | | cudTsdT | | | AAAGcAdTsdT | |
| 808 | XD- | X29172 | uuGGGAAuGcAAug | 686 | X29173 | UUGcAcAUUGcAU | 824 |
| | 09107 | | uGcAAdTsdT | | | UCCcAAdTsdT | |
| 838 | XD- | X29184 | ggcuGuAucAcAucG | 692 | X29185 | uAACCGAUGUGAu | 830 |
| | 09113 | | GuuAdTsdT | | | AcAGCCdTsdT | |
| 840 | XD- | X29188 | cuGuAucAcAucGgu | 694 | X29189 | AAuAACCGAUGUG | 832 |
| | 09115 | | uAuudTsdT | | | AuAcAGdTsdT | |
| 842 | XD- | X29192 | guAucAcAucGGuuA | 696 | X29193 | AAAAuAACCGAUG | 834 |
| | 09117 | | uuuudTsdT | | | UGAuACdTsdT | |
| 848 | XD- | X29200 | caucGGuuAuuuugGc | 700 | X29201 | UCCGCcAAAAuAA | 838 |
| | 09121 | | GGAdTsdT | | | CCGAUGdTsdT | |
| 855 | XD- | X29212 | uauuuuGGcGGAAuc | 706 | X29213 | AGAAGAUUCCGCc | 844 |
| | 09127 | | uucudTsdT | | | AAAAuAdTsdT | |
| 1012 | XD- | X29234 | ucAucuuuAcuAAau | 717 | X29235 | UGGcAUUuAGuAA | 855 |
| | 09138 | | GccAdTsdT | | | AGAUGAdTsdT | |
| 1057 | XD- | X29244 | gcccuuAuuuuccacGA | 722 | X29245 | CCUCGUGGAAAAu | 860 |
| | 09143 | | GGdTsdT | | | AAGGGCdTsdT | |
| 1067 | XD- | X29256 | uccAcGAGGAuGGa | 728 | X29257 | AUuAUUCcAUCCU | 866 |
| | 09149 | | AuAAudTsdT | | | CGUGGAdTsdT | |
| 1096 | XD- | X29266 | uaAuGAGGAAcAuu | 733 | X29267 | CCcAAAAUGUUCC | 871 |
| | 09154 | | uuGGGdTsdT | | | UcAUuAdTsdT | |
| 495 | XD- | X32257 | acAucuCGUucUcgG | 961 | X32336 | dTAAACCGAGAAC | 1002 |
| var1 | 10302 | | uUusa | | | gAGAUGUusu | |
| 498 | XD- | X32258 | ucUcGuUCUcGGuu | 962 | X32337 | dTGAuAAACCGAGa | 1003 |
| var1 | 10303 | | uAUcsa | | | ACGAGAusu | |
| 751 | XD- | X32259 | ugCuuuUUCuuAuuA | 963 | X32338 | dTGAGuAAuAAGAa | 1004 |
| var1 | 10304 | | cUcsa | | | AAAGcAusu | |
| 842 | XD- | X32260 | guAucACAUcGGuu | 964 | X32339 | dTAAAuAACCGAUg | 1005 |
| var1 | 10305 | | AuUusa | | | UGAuACusu | |
| 855 | XD- | X32261 | uaUuuuGGCgGAAu | 965 | X32340 | dTGAAGAUUCCGC | 1006 |
| var1 | 10306 | | cuUcsa | | | cAAAAuAusu | |
| 495 | XD- | X32262 | acAucucGuucucgGu | 966 | X32336 | dTAAACCGAGAAC | 1007 |
| var2 | 10307 | | uusa | | | gAGAUGUusu | |
| 498 | XD- | X32263 | ucucGuucucGGuuuA | 967 | X32337 | dTGAuAAACCGAGa | 1008 |
| var2 | 10308 | | ucsa | | | ACGAGAusu | |
| 751 | XD- | X32264 | ugcuuuuucuuAuuAcu | 968 | X32338 | dTGAGuAAuAAGAa | 1009 |
| var2 | 10309 | | csa | | | AAAGcAusu | |
| 842 | XD- | X32265 | guAucAcAucGGuuA | 969 | X32339 | dTAAAuAACCGAUg | 1010 |
| var2 | 10310 | | uuusa | | | UGAuACusu | |
| 855 | XD- | X32266 | uauuuuGGcgGAAuc | 970 | X32340 | dTGAAGAUUCCGC | 1011 |
| var2 | 10311 | | uucsa | | | cAAAAuAusu | |
| 495 | XD- | X32267 | acAucuCGUucUcgG | 971 | X32341 | UAAACCGAGAACg | 1012 |
| var3 | 10312 | | uUua(C6) | | | AGAUGUusu | |
| 498 | XD- | X32268 | ucUcGuUCUcGGuu | 972 | X32342 | UGAuAAACCGAGa | 1013 |
| var3 | 10313 | | uAUca(C6) | | | ACGAGAusu | |
| 751 | XD- | X32269 | ugCuuuUUCuuAuuA | 973 | X32343 | UGAGuAAuAAGAa | 1014 |
| var3 | 10314 | | cUca(C6) | | | AAAGcAusu | |
| 842 | XD- | X32270 | guAucACAUcGGuu | 974 | X32344 | UAAAuAACCGAUg | 1015 |
| var3 | 10315 | | AuUua(C6) | | | UGAuACusu | |
| 855 | XD- | X32271 | uaUuuuGGCgGAAu | 975 | X32345 | UGAAGAUUCCGCc | 1016 |
| var3 | 10316 | | cuUca(C6) | | | AAAAuAusu | |
| 489 | XD- | X32272 | cuGuccACAucUcguu | 976 | X32346 | dTAGAACGAGAUG | 1017 |
| var1 | 10317 | | Cusa | | | uGGAcAGusu | |
| 492 | XD- | X32273 | ucCAcAUCUcGUuc | 977 | X32347 | dTCCGAGAACGAG | 1018 |
| var1 | 10318 | | ucGgsa | | | aUGUGGAusu | |
| 533 | XD- | X32274 | cgGuGAAGAaGUca | 978 | X32348 | dTGUGGUGACUUC | 1019 |
| var1 | 10319 | | ccAcsa | | | uUcACCGusu | |
| 544 | XD- | X32275 | ucAccACCUuuUuuG | 979 | X32349 | dTAAUcAAAAAAG | 1020 |
| var1 | 10320 | | AUusa | | | gUGGUGAusu | |
| 590 | XD- | X32276 | ugAcGuGAAgcAAa | 980 | X32350 | dTCcAAUUUGCUUc | 1021 |
| var1 | 10321 | | uuGgsa | | | ACGUcAusu | |
| 693 | XD- | X32277 | ugCAAAAAGcuGA | 981 | X32351 | dTGcACUUcAGCUu | 1022 |
| var1 | 10322 | | aGuGcsa | | | UUUGcAusu | |
| 698 | XD- | X32278 | aaAGcuGAAguGcuu | 982 | X32352 | dTGUcAAGcACUUc | 1023 |
| var1 | 10323 | | GAcsa | | | AGCUUUusu | |
| 808 | XD- | X32279 | uuGGGAAUGcAAu | 983 | X32353 | dTUGcAcAUUGcAu | 1024 |
| var1 | 10324 | | guGCasa | | | UCCcAAusu | |
| 836 | XD- | X32280 | agGGcuGUAucAcau | 984 | X32354 | dTCCGAUGUGAuAc | 1025 |
| var1 | 10325 | | cGgsa | | | AGCCCUusu | |
| 838 | XD- | X32281 | ggCuGuAUCacAucG | 985 | X32355 | dTAACCGAUGUGA | 1026 |
| var1 | 10326 | | GUusa | | | uAcAGCCusu | |
| 840 | XD- | X32282 | cuGuAuCACauCGgu | 986 | X32356 | dTAuAACCGAUGUg | 1027 |
| var1 | 10327 | | uAusa | | | AuAcAGusu | |
| 848 | XD- | X32283 | caUcGGUUAuuUug | 987 | X32357 | dTCCGCcAAAAuAa | 1028 |
| var1 | 10328 | | GcGgsa | | | CCGAUGusu | |
| 1012 | XD- | X32284 | ucAucuUUAcuAAau | 988 | X32358 | dTGGcAUUuAGuAa | 1029 |
| var1 | 10329 | | GCcsa | | | AGAUGAusu | |
| 1057 | XD- | X32285 | gcCcuuAUUuuCcac | 989 | X32359 | dTCUCGUGGAAAA | 1030 |
| var1 | 10330 | | GAgsa | | | uAAGGGCusu | |
| 1067 | XD- | X32286 | ucCAcGAGGauGGa | 990 | X32360 | dTUuAUUCcAUCCu | 1031 |
| var1 | 10331 | | AuAasa | | | CGUGGAusu | |
| 508 | XD- | X32287 | ggUuuAUCAgAAAu | 991 | X32361 | dTUGGuAUUUCUGa | 1032 |
| var1 | 10332 | | AcCasa | | | UAAACCusu | |
| 545 | XD- | X32288 | caCcAcCUUuuUugA | 992 | X32362 | dTUAAUcAAAAAA | 1033 |
| var1 | 10333 | | uUasa | | | gGUGGUGusu | |
| 547 | XD- | X32289 | ccAccuUUUuuGAuu | 993 | X32363 | dTCAuAAUcAAAAa | 1034 |
| var1 | 10334 | | AUgsa | | | AGGUGGusu | |
| 569 | XD- | X32290 | cgGuGcUCCcuGucA | 994 | X32364 | dTUuAUGAcAGGGa | 1035 |
| var1 | 10335 | | uAasa | | | GcACCGusu | |
| 574 | XD- | X32291 | cuCccuGUCauAAau | 995 | X32365 | dTCAAAUUuAUGAc | 1036 |
| var1 | 10336 | | uUgsa | | | AGGGAGusu | |
| 580 | XD- | X32292 | guCAuAAAUuuGAc | 996 | X32366 | dTUcACGUcAAAUu | 1037 |
| var1 | 10337 | | GuGasa | | | UAUGACusu | |
| 1006 | XD- | X32293 | caGGAAUCAucUuu | 997 | X32367 | dTUAGuAAAGAUG | 1038 |
| var1 | 10338 | | AcUasa | | | aUUCCUGusu | |
| 1007 | XD- | X32294 | agGAAuCAUcuUuac | 998 | X32368 | dTUuAGuAAAGAUg | 1039 |
| var1 | 10339 | | uAasa | | | AUUCCUusu | |
| 1069 | XD- | X32295 | caCGAGGAUgGAA | 999 | X32369 | dTAAUuAUUCcAUc | 1040 |
| var1 | 10340 | | uAAUusa | | | CUCGUGusu | |
| 1079 | XD- | X32296 | gaAuAAUUUccAcac | 1000 | X32370 | dTAUUGUGUGGAA | 1041 |
| var1 | 10341 | | AAusa | | | aUuAUUCusu | |
| 1089 | XD- | X32297 | caCAcAAUAauGAg | 1001 | X32371 | dTGUUCCUcAUuAu | 1042 |
| var1 | 10342 | | GAAcsa | | | UGUGUGusu | |

After incubating for 24 hours, the treated cells were harvested and the remaining CCR2 mRNA level was measured in each condition and ranked. The remaining CCR2 mRNA level after the 24 hours treatment with each siRNA duplex was listed in Table 19. Among the 61 duplexes tested, 5 siRNAs and the variants thereof that have the most reduced CCR2 mRNA level were selected, including duplex XD-09048 and its variants, XD-10302, XD-10307 and CD-10321; duplex XD-09050 and its variants, XD-10303, XD-10308 and XD-10313; duplex XD-09098 and its variants, XD-10304, XD-10309 and XD-10314; duplex XD-09117 and its variants, XD-10305, XD-10310 and XD-10315; and duplex XD-09127 and its variants, XD-10306, XD-10311 and XD-10316. The top 20 siRNA duplexes their modification variants were also ranked, including XD-09043 and its variant XD-10317; XD-09045 and its variant XD-10318; XD-09060 and its variant XD-10319; XD-09062 and its variant XD-10320; XD-09086 and its variant XD-10321; XD-09094 and its variant XD-10322; XD-09095 and its variant XD-10323; XD-09107 and its variant XD-10324; XD-09112 and its variant XD-10325; XD-09113 and XD-10326; XD-09115 and its XD-10327; XD-09121 and its variant XD-10328; XD-09138 and its variant XD-10329; XD-09143 and its variant XD-10330; and XD-09149 and its variant XD-10331 (Table 18). The data also indicate several other siRNA modifications that result in significant reduction of expression, such as XD-10332, XD-10333, XD-10334, XD-10335, XD-10335, XD-10336, XD-10337, XD-10338, XD-10339, XD-10340, XD-10341 and XD-10342.

TABLE 19
CCR2 mRNA expression after siRNA treatment

| | Remaining | | Remaining | |
|---|---|---|---|---|
| | mRNA | | mRNA | |
| Duplex ID | (20 nM siRNA) | SD | (0.2 nM siRNA) | SD |
| XD-09048 | 0.0828811 | 0.01621 | 0.26202 | 0.02301 |
| XD-10302 | 0.0458746 | 0.00603 | 0.09770 | 0.01263 |
| XD-10307 | 0.0572924 | 0.00596 | 0.09821 | 0.00772 |
| XD-10312 | 0.05 | 0.00919 | 0.07835 | 0.00144 |
| XD-09050 | 0.08619 | 0.00859 | 0.15136 | 0.02523 |
| XD-10303 | 0.05225 | 0.00315 | 0.09107 | 0.01053 |
| XD-10308 | 0.09851 | 0.01143 | 0.09575 | 0.00460 |
| XD-10313 | 0.05177 | 0.00743 | 0.10762 | 0.01369 |
| XD-09098 | 0.11996 | 0.01069 | 0.22805 | 0.01252 |
| XD-10304 | 0.10572 | 0.02569 | 0.15910 | 0.02864 |
| XD-10309 | 0.107998 | 0.02183 | 0.16562 | 0.02625 |
| XD-10314 | 0.0771379 | 0.00892 | 0.14788 | 0.01315 |
| XD-09117 | 0.1027451 | 0.02382 | 0.21454 | 0.03092 |
| XD-10305 | 0.08 | 0.01474 | 0.13802 | 0.00828 |
| XD-10310 | 0.10160 | 0.00876 | 0.19002 | 0.01845 |
| XD-10315 | 0.06324 | 0.01587 | 0.12922 | 0.01160 |
| XD-09127 | 0.04650 | 0.00482 | 0.19543 | 0.05751 |
| XD-10306 | 0.04222 | 0.00768 | 0.14426 | 0.02314 |
| XD-10311 | 0.04622 | 0.00548 | 0.14122 | 0.02280 |
| XD-10316 | 0.04534 | 0.00900 | 0.12270 | 0.01968 |
| XD-09043 | 0.1212235 | 0.01143 | 0.50504 | 0.04412 |
| XD-10317 | 0.0625109 | 0.01082 | 0.97832 | 0.05761 |
| XD-09045 | 0.0968615 | 0.00805 | 0.54488 | 0.02852 |
| XD-10318 | 0.07 | 0.01043 | 0.21755 | 0.01514 |
| XD-09060 | 0.05680 | 0.00704 | 0.38861 | 0.01861 |
| XD-10319 | 0.05437 | 0.00452 | 0.33435 | 0.04273 |
| XD-09062 | 0.08611 | 0.00674 | 0.32090 | 0.02100 |
| XD-10320 | 0.05999 | 0.00241 | 0.12294 | 0.01148 |
| XD-09086 | 0.07023 | 0.00615 | 0.39757 | 0.03588 |
| XD-10321 | 0.07552 | 0.00698 | 0.23543 | 0.03086 |
| XD-09094 | 0.0856153 | 0.00829 | 0.20789 | 0.01513 |
| XD-10322 | 0.0760854 | 0.00779 | 0.17054 | 0.02017 |
| XD-09095 | 0.0900298 | 0.01120 | 0.21249 | 0.01983 |
| XD-10323 | 0.08 | 0.01189 | 0.17035 | 0.01813 |
| XD-09107 | 0.07630 | 0.00240 | 0.15302 | 0.01313 |
| XD-10324 | 0.08814 | 0.01035 | 0.14028 | 0.03762 |
| XD-09112 | 0.09623 | 0.00753 | 0.15433 | 0.02567 |
| XD-10325 | 0.10806 | 0.01872 | 0.14031 | 0.01675 |
| XD-09113 | 0.08627 | 0.01356 | 0.33789 | 0.07231 |
| XD-10326 | 0.07559 | 0.01524 | 0.12001 | 0.01492 |
| XD-09115 | 0.1418658 | 0.02075 | 0.18826 | 0.02735 |
| XD-10327 | 0.0584942 | 0.00428 | 0.13135 | 0.01794 |
| XD-09121 | 0.0745102 | 0.00592 | 0.11849 | 0.00961 |
| XD-10328 | 0.06 | 0.00448 | 0.09767 | 0.00895 |
| XD-09138 | 0.06331 | 0.01094 | 0.16493 | 0.02036 |
| XD-10329 | 0.05851 | 0.00823 | 0.11598 | 0.02222 |
| XD-09143 | 0.13552 | 0.01133 | 0.38856 | 0.04295 |
| XD-10330 | 0.08985 | 0.01329 | 0.16521 | 0.03149 |
| XD-09149 | 0.11750 | 0.02240 | 0.24068 | 0.02158 |
| XD-10331 | 0.10518 | 0.02421 | 0.24268 | 0.01657 |
| XD-10332 | 0.0479462 | 0.00567 | 0.28390 | 0.03031 |
| XD-10333 | 0.1228451 | 0.00433 | 0.19917 | 0.01957 |
| XD-10334 | 0.0515203 | 0.00568 | 0.09184 | 0.00562 |
| XD-10335 | 0.07 | 0.00693 | 0.26358 | 0.02275 |
| XD-10336 | 0.13570 | 0.01136 | 0.23667 | 0.01347 |
| XD-10337 | 0.06058 | 0.01142 | 0.19522 | 0.02186 |
| XD-10338 | 0.05468 | 0.00741 | 0.12157 | 0.01297 |
| XD-10339 | 0.07645 | 0.00934 | 0.13829 | 0.02922 |
| XD-10340 | 0.12908 | 0.01606 | 0.21778 | 0.02301 |
| XD-10341 | 0.07806 | 0.01851 | 0.14420 | 0.01470 |
| XD-10342 | 0.1353099 | 0.0149615 | 0.3257299 | 0.0421325 |
| XD-00194 | 0.983 | 0.126 | 0.99921 | 0.13987 |
| (Neg. control) | | | | |
| XD-00379 | 0.006 | 0.002 | 0.04388 | 0.01031 |
| (Luc; Post. | | | | |
| Control) | | | | |

Dose Response of Selected CCR2 siRNA Duplexes and Variants

CSF1R siRNA duplexes and variants that resulted in a significant reduction of CSF1R mRNA level in the dual dose screening were selected and further tested for dose responses. Hepa 1-6 cells were cultured and maintained in 96-well plates at a density of 15,000 cells per well. Hepa 1-6 cells were transfected with CCR2 siRNA duplexes and variants selected from the previous dual dose screening, at various concentrations using Lipofectomine® 2000 (0.5 μl/well). The doses for each CCR2 siRNA duplex included 50 nM, 10 nM, 2.0 nM, 0.40 nM, 0.08 nM, 1.6×10⁻² nM, 3.2×10⁻³ nM, 6.4×10⁻⁴ nM, 1.28×10⁻⁴ nM, and 2.6×⁻⁵ nM. Following incubation of 24 hours, the treated cells were harvested and the remaining CCR2 mRNA level was measured in each condition. The IC₅₀ value of each CCR2 duplex was determined as shown in Table 20 and each dose response curve is shown in FIG. 1D.

TABLE 20
IC₅₀ and IC₈₀ of CCR2 siRNA duplexes

| siRNA Duplex ID | IC₅₀ (nM) | IC₈₀ (nM) | Maximal KD |
|---|---|---|---|
| XD-10311 | 0.007 | 0.056 | 93% |
| XD-10305 | 0.007 | 0.045 | 91% |
| XD-09098 | 0.028 | 0.246 | 87% |
| XD-10309 | 0.014 | 0.111 | 90% |
| XD-10303 | 0.004 | 0.030 | 94% |
| XD-10308 | 0.005 | 0.021 | 90% |
| XD-10302 | 0.006 | 0.030 | 94% |
| XD-10307 | 0.007 | 0.032 | 93% |
| XD-10312 | 0.005 | 0.035 | 95% |
| XD-10329 | 0.006 | 0.041 | 92% |
| XD-09121 | 0.009 | 0.062 | 93% |
| XD-10328 | 0.006 | 0.038 | 94% |
| XD-10320 | 0.012 | 0.097 | 93% |
| XD-10326 | 0.010 | 0.087 | 91% |

### Example 7: Dose Response of Combined CSF1R and CCR2 siRNAs

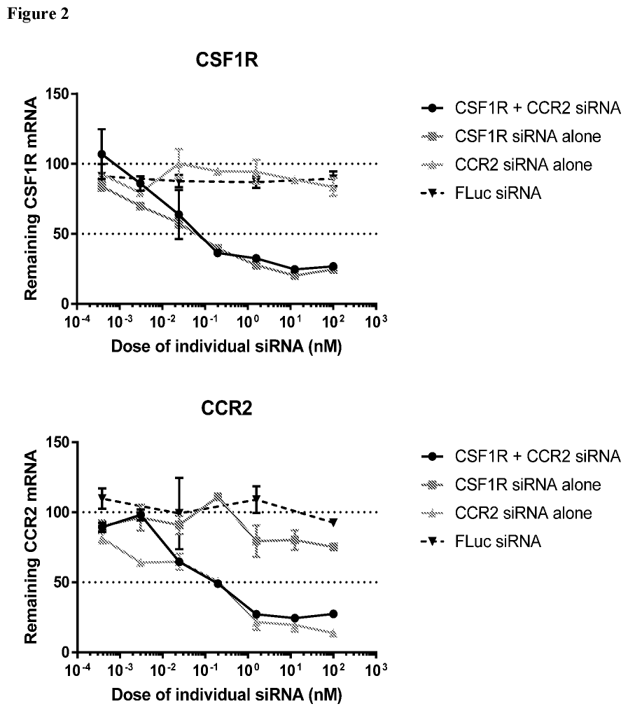

Human monocytic THP-1 cells were cultured and maintained in 96-well plates at a density of 25,000 cells per well. THP-1 monocytes were transfected with a combination of CSF1R duplex (XD-09016) and CCR2 duplex (XD-09098), CSF1R duplex (XD-09016) alone, CCR2 duplex (XD-09098) alone, or Firefly luciferase siRNA (FLuc, XD-00194) using Lipofectamine® 2000 (0.5 μl/well). After incubating for 24 hours, the cells were harvested and the remaining CSF1R, CCR2, and GAPDH mRNA levels were measured using a branched DNA (bDNA) assay according to the manufacturer's instructions (QuantiGene® SinglePlex Gene Expression Assay, ThermoFisher; CSF1R probe: SA-14861; CCR2 probe: SA-3020026; GAPDH probe: SA-10001). The data are shown in FIG. 2 plotted as individual siRNA concentration (nM) vs. remaining mRNA (ratio of target gene to GAPDH, normalized to mock-transfected cells) and represents the mean of duplicates, +/−standard deviation. The results demonstrated that silencing of CSF1R or CCR2 using a combination of siRNAs was as effective as silencing CSF1R or CCR2 using individual siRNAs because no significant difference was observed in the silencing of CSF1R or CCR2 when the siRNAs were transfected in combination with each other as opposed to transfection alone.

### Example 8: LNP-Formulated CSF1R and CCR2 siRNAs Administered Intraperitoneally in Mice

C57BL/6 mice (7-8 weeks old, female, n=5 per group) were dosed intraperitoneally (2 mg/kg by total siRNA) of LNPs on Days −4, −1, and 1. LNPs were synthesized at a composition of 50:10:38.5: 1.5 molar ratio of C12-200:1,2-distearoyl-sn-glycero-3-phosphocholine (DSPC): cholesterol: 1,2-dimyristoyl-sn-glycero-3-phosphoethanolamine-N-[methoxy(polyethylene glycol)-2000] (C14 PEG-2000) and a total lipid: total siRNA weight ratio of 9:1. LNPs were formulated with equimolar ratios of either: a) mCSF1R+Luc siRNAs, b) mCCR2+Luc siRNAs, c) mCSF1R+mCCR2 siRNAs, or d) Luc siRNA. Mouse siRNA sequences are listed in Table 21.

TABLE 21
Mouse siRNA sequences

| Target | Sense Sequence | Antisense Sequence |
|---|---|---|
| mCSF1R | cuAcucAAcuuucuccGAAdTsdT | UUCGGAGAAAGUUGAGuAGdTsdT |
| mCCR2 | uGcuAAAcGucucuGcAAAdTsdT | UUUGcAGAGACGUUuAGcAdTsdT |
| Luc | cuuAcGcuGAGuAcuucGAdTsdT | UCGAAGuACUcAGCGuAAGdTsdT |

On Day 0, mice were injected intraperitoneally with 2 mL of 3% thioglycollate broth (Difco Fluid Thioglycollate medium, BD 225650) to induce macrophage migration to the peritoneum. On Day 3, mice were sacrificed and their peritoneal macrophages were collected. Single cell suspensions were generated and analyzed via flow cytometry (Table 22).

TABLE 22
Flow cytometry antibodies used for assays

| Color | Target | Clone | Manufacturer |
|---|---|---|---|
| BV421 | mCCR2 | SA203G11 | Biolegend |
| BV510 | Viability | n/a | ThermoFisher |
| BV605 | mTCR-B, mCD19, | H57-597, 6D5, | Biolegend (for all |
| | mNK1.1 | PK136 | clones) |
| BV711 | mCD45 | 30-F11 | Biolegend |
| PerCP/Cy5.5 | mLy-6C | HK1.4 | Biolegend |
| PE | mCSF1R | AFS98 | Biolegend |
| PE/Dazzle594 | mLy-6G | 1A8 | Biolegend |
| AF700 | mF4/80 | CI:A3-1 | Bio Rad |
| APC/Fire750 | mCD11b | M1/70 | Biolegend |

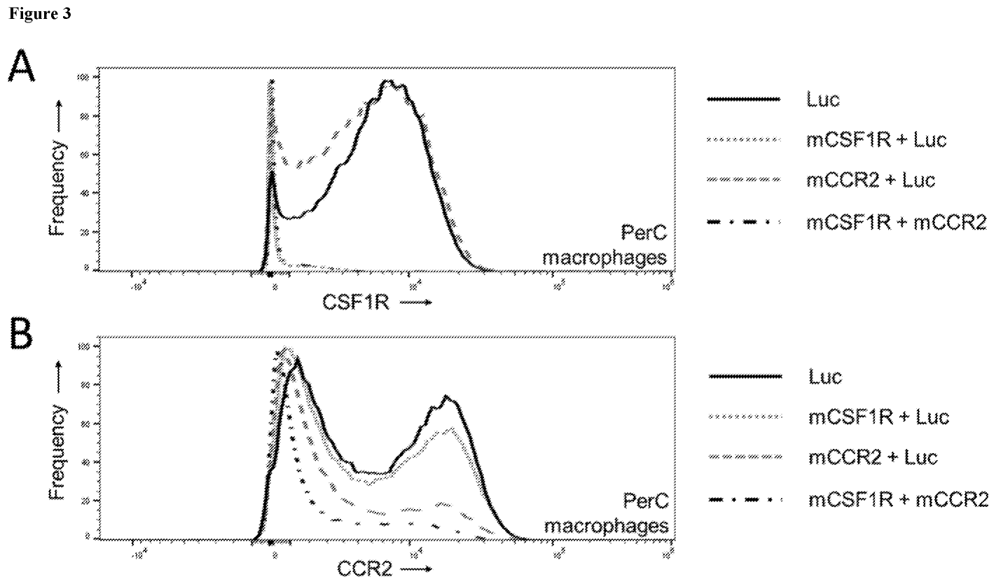

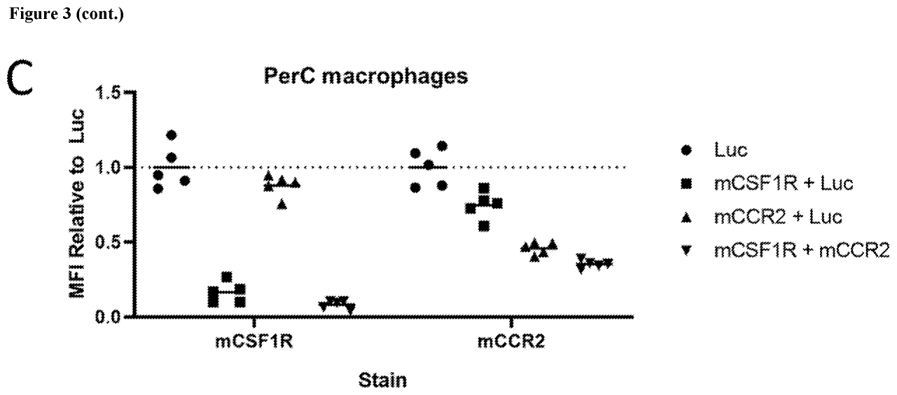

Peritoneal macrophages were gated by singlet, live, mCD45+, mTCR-B−, mCD19−, mNK1.1−, mLy-6G−, mCD11b+, and mF4/80+ criteria. Then, mCSF1R/mCCR2 expression was graphed and quantified (FIG. 3). The results demonstrated that silencing of mCSF1R and mCCR2 were simultaneously achieved in peritoneal macrophages in mice following intraperitoneal administration of siRNA-LNPs. Additionally, FIG. 3C demonstrates that using a combination of siRNAs was as at least as effective as, and believed to be more effective than, silencing mCSF1R or mCCR2 using individual siRNAs.

### Example 9: LNP-Formulated CSF1R and CCR2 siRNAs Administered Intravenously in Mice

C57BL/7 mice (7-8 weeks old, female, n=3 per group) were dosed intravenously (1.5 mg/kg by total siRNA) of LNPs on Days −4, −1, and 1. LNPs were synthesized at a composition of 50:10: 38.5:1.5 molar ratio of C12-200:1,2-distearoyl-sn-glycero-3-phosphocholine (DSPC): cholesterol: 1,2-dimyristoyl-sn-glycero-3-phosphoethanolamine-N-[methoxy(polyethylene glycol)-2000] (C14 PEG-2000) and a total lipid: total siRNA weight ratio of 9:1. LNPs were formulated with equimolar ratios of either: a) mCSF1R+mCCR2 siRNAs or b) Luc siRNA. Mouse siRNA sequences are listed in Table 21 described above.

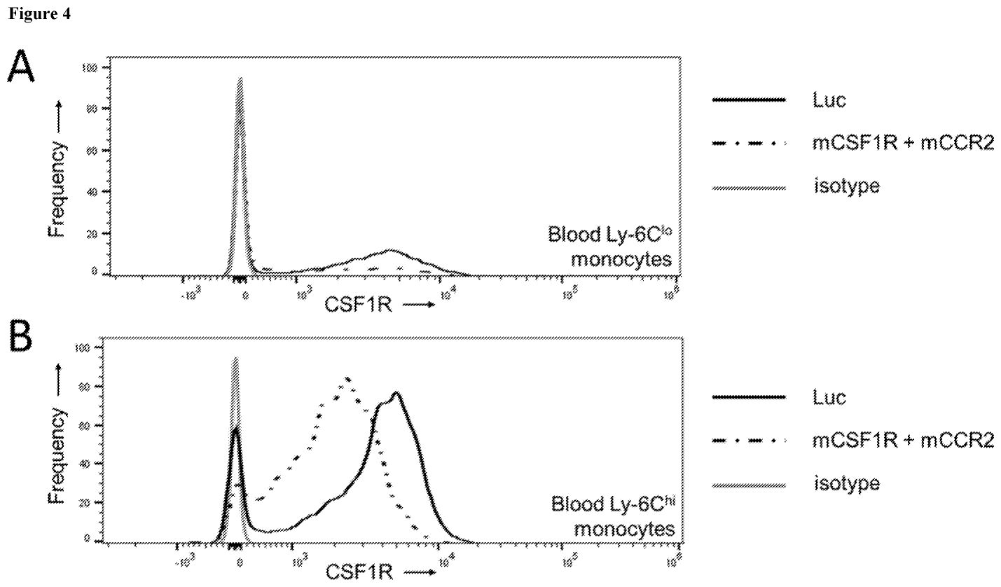

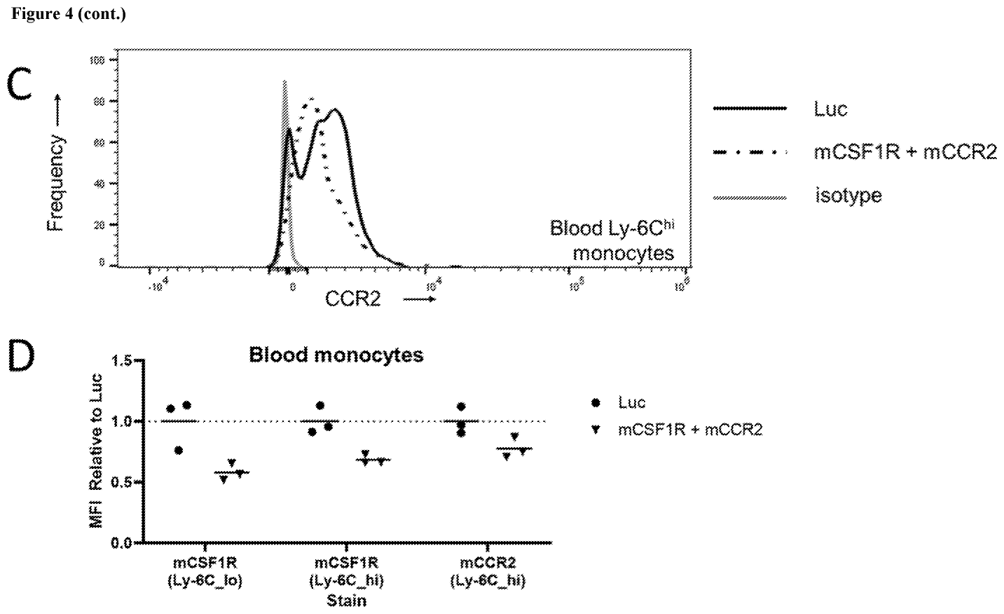

On Day 0, mice were injected intraperitoneally with 2 mL of 3% thioglycollate broth (Difco Fluid Thioglycollate medium, BD 225650) to induce macrophage migration to the peritoneum (e.g., thyglycollate peritonitis model because it was determined that a standard lipopolysaccharide (LPS) peritonitis model did not produce macrophages expressing both CCR2 and CSF1R as CCR2 is quickly downregulated). On Day 3, mice were sacrificed and their blood was collected. Single cell suspensions were generated and analyzed via flow cytometry (Table 22; described above). Blood monocytes were gated by singlet, live, mCD45+, mTCR-B−, mCD19-, mNK1.1-, mCD11b+, mLy-6G-criteria and were then gated separately as Ly-6C^{hi} and Ly-6C^{lo} monocytes (as well-known gating criteria and described, for example, in Leuschner et al. (2012) Nat. Biotechnol. 29:1005-1010 and Rose et al. (2012) Cytometry A 81:343-350) since mCCR2 expression is associated with pro-inflammatory Ly-6C^{hi} monocytes but not Ly-6C^{lo} monocytes. Then, mCSF1R/mCCR2 expression was graphed and quantified (FIG. 4). The results demonstrated that silencing of mCSF1R on blood monocytes and mCCR2 on Ly-6C^{hi} blood monocytes was simultaneously achieved using the combination of siRNAs in mice following intravenous administration of siRNA-LNPs.

### Example 10: Synergistic Silencing of CSF1R and CCR2 in a Model In Vitro Reporter System

Regions of CSF1R (NM_005211.3, nucleotide regions: 1030-1108, 2844-2922, 3019-3097, 3887-3965) and CCR2 (NM_001123396.2, nucleotide regions: 465-546, 721-799, 818-896, 982-1060) were cloned into a psiCHECK™-2 (Promega) vector downstream of the Renilla luciferase (RLuc) reporter gene. This vector also contains a secondary Firefly luciferase (FLuc) reporter cassette as an internal control. Silencing of either CSF1R or CCR2 in cells expressing this plasmid results in proportional silencing of RLuc and can be normalized for cell count by FLuc. Thus, with this reporter system, both CSF1R and CCR2 silencing can be measured together with a single readout to quantify potential synergistic silencing of the combination of CSF1R and CCR2 siRNAs.

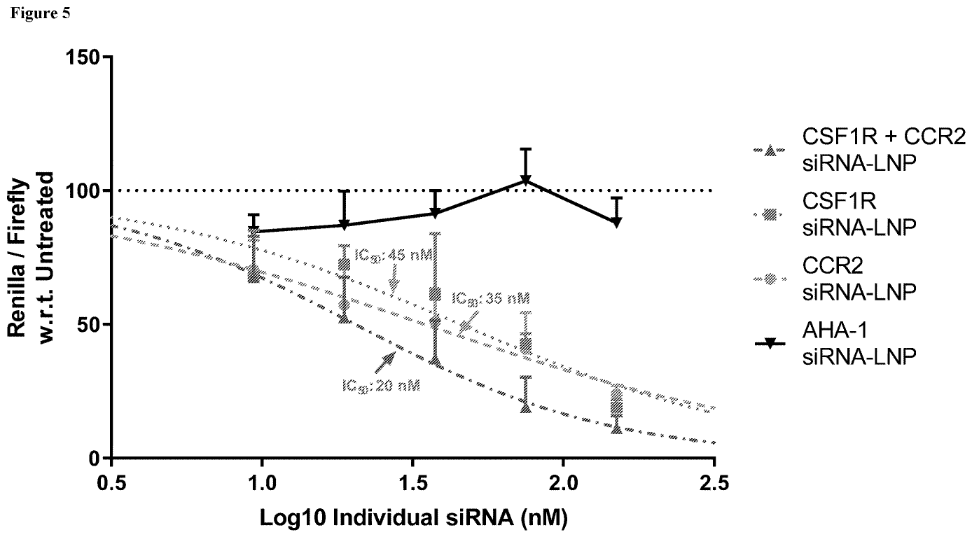

CHO cells were plated at 30,000 cells/well in a 96-well plate and transfected with 200 ng of the psiCHECK™-2 plasmid and 0.5 uL of Lipofectamine® 2000. After 24 hr of incubation at 37° C., the media was replaced. LNPs were synthesized at a composition of 50: 10:38.5:1.5 molar ratio of C12-200:1,2-distearoyl-sn-glycero-3-phosphocholine (DSPC): cholesterol: 1,2-dimyristoyl-sn-glycero-3-phosphoethanolamine-N-[methoxy(polyethylene glycol)-2000] (C14 PEG-2000) and a total lipid: total siRNA weight ratio of 9:1.; then, LNPs were added to each well with varying individual siRNA concentrations as shown in FIG. 5. LNPs contained either CSF1R siRNA (XD-09016), CCR2 siRNA (XD-09098), CSF1R+CCR2 siRNA, or AHA-1 siRNA. AHA-1 siRNA (Table 23), which targets the housekeeping gene AHA-1, was used as a negative control.

TABLE 23
AHA-1 siRNA sequence

| Target | Sense Sequence | Antisense Sequence |
|---|---|---|
| ABA-1 | ggAuGAAGuGGAGauuAGudTsdT | ACuAAUCUCcACUUcAUCCdTsdT |

After 24 hr, a Dual-Glo® Luciferase Assay (Promega) was performed according to the manufacturer's instructions. The Renilla luminescence was normalized by the Firefly luminescence, and this ratio was then normalized to plasmid-transfected untreated cells on the y-axis; the individual (not total) CSF1R or CCR2 siRNA concentration of each LNP was plotted on the x-axis (FIG. 5).

The results demonstrated that dose-dependent silencing of CSF1R and CCR2 was achieved with the single and combination siRNA-LNPs. A four-point sigmoidal curve (GraphPad Prism) was fitted to the data to determine IC50s. CSF1R siRNA-LNPs and CCR2 siRNA-LNPs had IC₅₀s of approximately 45 nM and 35 nM, respectively. The combination CSF1R+CCR2 siRNA-LNPs had an IC50 of approximately 20 nM. The IC50 of the combination siRNAs is approximately half of the single siRNAs, thereby demonstrating synergistic silencing of CSF1R and CCR2 in this model in vitro reporter system with a single endpoint.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web and/or the National Center for Biotechnology Information (NCBI) on the World Wide Web.

### EQUIVALENTS AND SCOPE

The details of one or more embodiments encompassed by the present invention are set forth in the description above. Although the preferred materials and methods have been described above, any materials and methods similar or equivalent to those described herein can be used in the practice or testing of embodiments encompassed by the present invention. Other features, objects and advantages related to the present invention are apparent from the description. Unless defined otherwise, all technical and scientific terms used herein have the same meaning as commonly understood by one of ordinary skill in the art to which this invention belongs. In the case of conflict, the present description provided above will control.

Those skilled in the art will recognize, or be able to ascertain using no more than routine experimentation, many equivalents to the specific embodiments encompassed by the present invention described herein. The scope of the present invention is not intended to be limited to the description provided herein and such equivalents are intended to be encompassed by the appended claims.

The articles “a” and “an” are used herein to refer to one or to more than one (i.e. to at least one) of the grammatical object of the article unless indicated to the contrary or otherwise evident from the context. By way of example, “an element” means one element or more than one element. Claims or descriptions that include “or” between one or more members of a group are considered satisfied if one, more than one, or all of the group members are present in, employed in, or otherwise relevant to a given product or process unless indicated to the contrary or otherwise evident from the context. The present invention includes embodiments in which exactly one member of the group is present in, employed in, or otherwise relevant to a given product or process. The present invention also includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments encompassed by the present invention, to the tenth of the unit of the lower limit of the range, unless the context clearly dictates otherwise.

In addition, it is to be understood that any particular embodiment of the present invention that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions encompassed by the present invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes can be made within the purview of the appended claims without departing from the true scope and spirit encompassed by the present invention in its broader aspects.

While the present invention has been described at some length and with some particularity with respect to several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present invention.

What is claimed is:

1. A composition comprising a) at least one siRNA molecule that hybridizes to a nucleic acid molecule encoding CCR2, b) at least one siRNA molecule that hybridizes to a nucleic acid molecule encoding CSF1R, or c) a combination of a) and b).

2. The composition of claim 1, wherein the at least one siRNA molecule that hybridizes to the nucleic acid molecule encoding CCR2 comprises a sense strand having a nucleic acid sequence selected from SEQ ID NO: 6 to SEQ ID NO: 67 and an anti-sense strand having a nucleic acid sequence selected from SEQ ID NO: 68 to SEQ ID NO: 129.

3. The composition of claim 1 or 2, wherein the at least one siRNA molecule that hybridizes to the nucleic acid molecule encoding CSF1R comprises a sense strand having a nucleic acid sequence selected from SEQ ID NO: 130 to SEQ ID NO 248 and an anti-sense strand having a nucleic acid sequence selected from SEQ ID NO: 249 to SEQ ID NO: 367.

4. The composition of any one of claims 1-3, wherein the at least one siRNA molecule that hybridizes to the nucleic acid molecule encoding CCR2 or CSF1R further comprise at least one modification.

5. The composition of claim 4, wherein the modification is a modification to the sugar moiety of the nucleic acid sequence, a nucleobase modification, an internucleoside linker modification, an artificial nucleotide, an end cap modification, or any combinations thereof.

6. The composition of claim 4 or 5, wherein the modification locates in the sense strand of the at least one siRNA molecule.

7. The composition of claim 4 or 5, wherein the modification locates in the anti-sense strand of the at least one siRNA molecule.

8. The composition of claim 4 or 5, wherein the modification locates in the sense and anti-sense strands of the at least one siRNA molecule.

9. The composition of claim 4 or 5, wherein the at least one siRNA molecule that hybridizes to the nucleic acid molecule encoding CCR2 comprises a sense strand having a modified nucleic acid sequence selected from SEQ ID NO: 368 to SEQ ID NO: 486 and SEQ ID NO: 883 to SEQ ID NO: 921, and an anti-sense strand having a modified nucleic acid sequence selected from SEQ ID NO: 487 to SEQ ID NO: 605 and SEQ ID NO: 922 to SEQ ID NO: 960.

10. The composition of claim 4, 5, or 9, wherein the at least one siRNA molecule that hybridizes to the nucleic acid molecule encoding CCR2 comprises a sense strand having a modified nucleic acid sequence selected from SEQ ID NO: 606 to SEQ ID NO: 743 and SEQ ID NO: 961 to SEQ ID NO: 1001, and an anti-sense strand having a modified nucleic acid sequence selected from SEQ ID NO: 744 to SEQ ID NO: 881 and SEQ ID NO: 1002 to SEQ ID NO: 1042.

11. A composition comprising a) at least one siRNA duplex that hybridizes to a nucleic acid molecule encoding CCR2, b) at least one siRNA duplex that hybridizes to a nucleic acid molecule encoding CSF1R, or c) a combination of a) and b),
wherein the at least one siRNA duplex that hybridizes to the nucleic acid molecule encoding CCR2 comprises a sense strand having a nucleic acid sequence selected from SEQ ID NO: 6 to SEQ ID NO: 67, or a modified nucleic acid sequence selected from SEQ ID NO: 606 to SEQ ID NO: 743, or a modification variant selected from SEQ ID NO: 961 to SEQ ID NO: 1001, and an anti-sense strand having a nucleic acid sequence selected from SEQ ID NO: 68 to SEQ ID NO: 129, or a modified nucleic acid sequence selected from SEQ ID NO: 744 to SEQ ID NO: 881, or a modification variant selected from SEQ ID NO: 1002 to SEQ ID NO: 1042; and/or
wherein the at least one siRNA duplex that hybridizes to the nucleic acid molecule encoding CSF1R comprises a sense strand having a nucleic acid sequence selected from SEQ ID NO: 130 to SEQ ID NO: 248, or a modified nucleic acid sequence selected from SEQ ID NO: 368 to SEQ ID NO: 486, or a modification variant selected from SEQ ID NO: 883 to SEQ ID NO: 921, and an anti-sense strand having a nucleic acid sequence selected from SEQ ID NO: 249 to SEQ ID NO: 367, or a modified nucleic acid sequence selected from SEQ ID NO: 487 to SEQ ID NO: 605, or a modification variant selected from SEQ ID NO: 922 to SEQ ID NO: 960.

12. The composition of claim 11, wherein the at least one siRNA duplex that hybridizes to the nucleic acid molecule encoding CCR2 is duplex XD-09048, XD-09050, XD-09098, XD-09117, XD-09127, XD-09043, XD-09045, XD-09060, XD-09062, XD-09086, XD-09094, XD-09095, XD-09107, XD-09112, XD-09113, XD-09115, XD-09121, XD-09138, XD-09143, or XD-09149, or variants thereof.

13. The composition of claim 12, wherein the at least one siRNA duplex that hybridizes to the nucleic acid molecule encoding CCR2 is duplex XD-09048, XD-09050, XD-09098, XD-09117 or XD-09127, or variants thereof.

14. The composition of any one of claims 11-13, wherein the at least one siRNA duplex that hybridizes to the nucleic acid molecule encoding CSF1R is duplex XD-08944, XD-08947, XD-08988, XD-08993 or XD-08916, XD-08917, XD-08922, XD-08923, XD-08936, XD-08963, XD-08969, XD-08975, XD-08982, XD-08985, XD-08986, XD-08989, XD-09003, XD-09006, XD-09015, or XD-09021, or variants thereof.

15. The composition of claim 14, wherein the at least one siRNA duplex that hybridizes to the nucleic acid molecule encoding CSF1R is duplex XD-08944, XD-08947, XD-08988, XD-08993 or XD-08916, or variants thereof.

16. The composition of any one of claims 1-15, wherein the composition further comprises a lipid and/or a lipidoid.

17. The composition of claim 16, wherein the lipidoid is of Formula (VI): 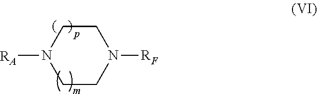
wherein:
p is an integer between 1 and 3, inclusive;
m is an integer between 1 and 3, inclusive;
R_{A} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl; 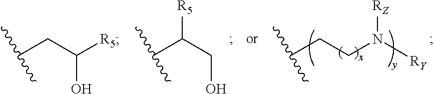
R_{F} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl; 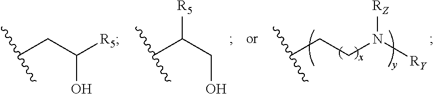
each occurrence of R₅ is independently hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; or substituted or unsubstituted heteroaryl;
wherein, at least one of R_{A}, R_{F}, R_{Y}, and R_{Z} is 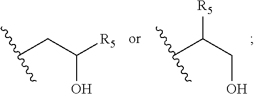
each occurrence of x is an integer between 1 and 10, inclusive;
each occurrence of y is an integer between 1 and 10, inclusive;
each occurrence of R_{Y} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl; 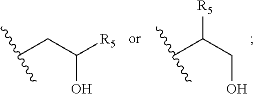
each occurrence of R_{Z} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl: 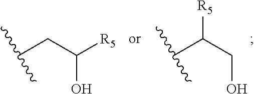
or a pharmaceutically acceptable salt thereof.

18. The composition of claim 17, wherein p is 1.

19. The composition of claim 17 or 18, wherein m is 1.

20. The composition of any one of claims 17-19, wherein each of p and m is 1.

21. The composition of any one of claims 17-20, wherein R_{F} is

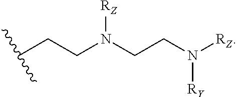

22. The composition of any one of claims 17-21, wherein R_{A} is

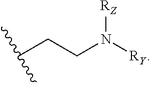

23. The composition of claim 17, wherein the compound of Formula (VI) is of the formula: 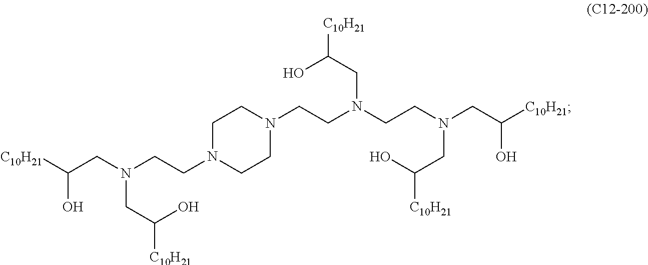
or a salt thereof.

24. The composition of any one of claims 17-23, wherein the composition is in the form a lipid nanoparticle.

25. The composition of claim 24, wherein the lipid nanoparticle comprises about 1.0% to about 60.0% by mole of C12-200.

26. The composition of claim 24 or 25, wherein the lipid nanoparticle further comprises one or more co-lipids.

27. The composition of claim 26, wherein each co-lipid is selected from disteroylphosphatidyl choline (DSPC), cholesterol, and DMG-PEG.

28. The composition of claim 27, wherein the concentration of DSPC is about 1.0% to about 20.0% by mole.

29. The composition of claim 27 or 28, wherein the concentration of cholesterol is about 10.0% to about 50.0% by mole.

30. The composition of any one of claims 27-29, wherein the concentration of DMG-PEG is about 0.1% to about 5.0% by mole.

31. The composition of any one of claims 27-30, wherein DSPC is present a concentration of about 1.0% to about 20.0% by mole; cholesterol is present at a concentration of about 10.0% to about 50.0% by mole; and DMG-PEG is present a concentration of about 0.1% to about 5.0% by mole.

32. The composition of claim 31, wherein C12-200, DSPC, cholesterol, and DMG-PEG are present at a ratio of 50%:10%:38.5%:1.5%, respectively.

33. The composition of any one of claims claim 16-32, wherein the lipids and lipidoids of the LNP compared to the siRNA molecules are present at a ratio from about 20:1 to about 5:1 by weight.

34. The composition of claim 33, wherein the lipids and lipidoids of the LNP compared to the siRNA molecules are present at a ratio of 9:1 by weight.

35. The composition of any one of claims 1-34, wherein the composition is in a pharmaceutically acceptable formulation.

36. A method of generating a myeloid-derived cell having an increased inflammatory phenotype after contact with at least one composition of any one of claims 1-35, comprising contacting the myeloid-derived cell with an effective amount of the at least one composition.

37. The method of claim 36, wherein the myeloid-derived cell having an increased inflammatory phenotype exhibits one or more of the following after contact with the at least one composition:
a) increased expression of cluster of differentiation 80 (CD80), CD86, WICK MEM, interleukin 1-beta (IL-1β), IL-6, CCL3, CCL4, CXCL10, CXCL9, GM-CSF and/or tumor necrosis factor alpha (TNF-α);
b) decreased expression of CD206, CD163, CD16, CD53, VSIG4, PSGL-1, TGFb and/or IL-10;
c) increased secretion of at least one cytokine or chemokine selected from the group consisting of IL-1β, TNF-α, IL-12, IL-18, GM-CSF, CCL3, CCL4, and IL-23;
d) increased ratio of expression of IL-1β, IL-6, and/or TNF-α to expression of IL-10;
e) increased CD8+ cytotoxic T cell activation;
f) increased recruitment of CD8+ cytotoxic T cell activation;
g) increased CD4+ helper T cell activity;
h) increased recruitment of CD4+ helper T cell activity;
i) increased NK cell activity;
j) increased recruitment of NK cell;
k) increased neutrophil activity;
l) increased macrophage activity; and/or
m) increased spindle-shaped morphology, flatness of appearance, and/or number of dendrites, as assessed by microscopy.

38. The method of claim 36 or 37, wherein the myeloid-derived cell contacted with the at least one composition are comprised within a population of cells and the at least one composition increases the number of Type 1 and/or M1 macrophages, and/or decreases the number of Type 2 and/or M2 macrophages, in the population of cells.

39. The method of any one of claims 36-38, wherein the myeloid-derived cell contacted with the at least one composition is comprised within a population of cells and the at least one composition increases the ratio of i) to ii), wherein i) is Type 1 and/or M1 macrophages and ii) is Type 2 and/or M2 macrophages in the population of cells.

40. The method of any one of claims 36-39, wherein the myeloid-derived cell is contacted in vitro or ex vivo.

41. The method of claim 40, wherein the myeloid-derived cell is a primary myeloid-derived cell.

42. The method of any one of claims 36-41, wherein the myeloid-derived cell is purified and/or cultured prior to contact with the at least one composition.

43. The method of any one of claims 36-39, wherein the myeloid-derived cell is contacted in vivo.

44. The method of claim 43, wherein the myeloid-derived cell is contacted in vivo by systemic, peritumoral, or intratumoral administration of the composition.

45. The method of claim 43 or 44, wherein the myeloid-derived cell is contacted in a subject in need thereof, optionally wherein the contact is in a tissue microenvironment.

46. The method of any one of claims 36-45, further comprising contacting the myeloid-derived cell with at least one additional therapeutic agent.

47. The method of claim 46, wherein the at least one additional therapeutic agent is an antagonist of CCL2 and/or an antagonist of CSF1.

48. The method of claim 46 or 47, wherein the at least one additional therapeutic agent comprises an immunotherapeutic agent that modulates the inflammatory phenotype, optionally wherein the immunotherapeutic agent comprises an immune checkpoint inhibitor, immune-stimulatory agonist, inflammatory agent, cells, a cancer vaccine, and/or a virus.

49. A method of increasing an inflammatory phenotype of myeloid-derived cells in a subject after contact with at least one composition of any one of claims 1-35, comprising administering to the subject an effective amount of the at least one composition that contacts the myeloid-derived cells.

50. The method of claim 49, wherein the myeloid-derived cells having the increased inflammatory phenotype exhibit one or more of the following after contact with the at least one composition:
a) increased expression of cluster of differentiation 80 (CD80), CD86, WICK MEM, interleukin 1-beta (IL-1β), IL-6, CCL3, CCL4, CXCL10, CXCL9, GM-CSF and/or tumor necrosis factor alpha (TNF-α);
b) decreased expression of CD206, CD163, CD16, CD53, VSIG4, PSGL-1 and/or IL-10;
c) increased secretion of at least one cytokine selected from the group consisting of IL-1β, TNF-α, IL-12, IL-18, and IL-23;
d) increased ratio of expression of IL-1β, IL-6, and/or TNF-α to expression of IL-10;
e) increased CD8+ cytotoxic T cell activation;
f) increased CD4+ helper T cell activity;
g) increased NK cell activity;
h) increased neutrophil activity;
i) increased macrophage activity; and/or
j) increased spindle-shaped morphology, flatness of appearance, and/or number of dendrites, as assessed by microscopy.

51. The method of claim 49 or 50, wherein the at least one composition increases the number of Type 1 and/or M1 macrophages, decreases the number of Type 2 and/or M2 macrophages, and/or increases the ratio of i) to ii), wherein i) is Type 1 and/or M1 macrophages and ii) is Type 2 and/or M2 macrophages, in the subject.

52. The method of any one of claims 49-51, wherein the number and/or activity of cytotoxic CD8+ T cells in the subject is increased after administration of the at least one composition.

53. The method of any one of claims 49-52, wherein the at least one composition is administered systemically, peritumorally, or intratumorally.

54. The method of any one of claims 49-53, wherein the at least one composition contacts the myeloid-derived cells in a tissue microenvironment.

55. The method of any one of claims 49-54, further comprising contacting the myeloid-derived cells with at least one additional therapeutic agent.

56. The method of claim 55, wherein the at least one additional therapeutic agent is an antagonist of CCL2 and/or an antagonist of CSF1.

57. The method of claim 55 or 56, wherein the at least one additional therapeutic agent comprises an immunotherapeutic agent that modulates the inflammatory phenotype, optionally wherein the immunotherapeutic agent comprises an immune checkpoint inhibitor, immune-stimulatory agonist, inflammatory agent, cells, a cancer vaccine, and/or a virus.

58. The method of claim 57, wherein the immune checkpoint is selected from the group consisting of PD-1, PD-L1, PD-L2, and CTLA-4.

59. The method of claim 58, wherein the immune checkpoint is PD-1.

60. The method of any one of claims 55-59, wherein the at least one additional therapeutic agent or regimen is administered before, concurrently with, or after the at least one composition.

61. A method of sensitizing cancer cells in a subject to cytotoxic CD8+ T cell-mediated killing and/or immune checkpoint therapy comprising administering to the subject a therapeutically effective amount of at least one composition of any one of claims 1-35 for contacting myeloid-derived cells in the subject.

62. The method of claim 61, wherein the at least one composition is administered systemically, peritumorally, or intratumorally.

63. The method of claim 61 or 62, further comprising treating the cancer in the subject by administering to the subject an effective amount of at least one additional therapeutic agent.

64. The method of claim 63, wherein the at least one additional therapeutic agent is an antagonist of CCL2 and/or an antagonist of CSF1.

65. The method of claim 63 or 64, wherein the at least one additional therapeutic agent comprises an immunotherapeutic agent that modulates the inflammatory phenotype of the myeloid-derived cells, optionally wherein the immunotherapeutic agent comprises an immune checkpoint inhibitor, immune-stimulatory agonist, inflammatory agent, cells, a cancer vaccine, and/or a virus.

66. The method of claim 65, wherein the immune checkpoint is selected from the group consisting of PD-1, PD-L1, PD-L2, and CTLA-4.

67. The method of claim 66, wherein the immune checkpoint is PD-1.

68. The method of claim 67, wherein the at least one additional therapeutic agent or regimen is administered before, concurrently with, or after the at least one composition.

69. The method of any one of claims 61-68, wherein the at least one composition reduces the number of proliferating cells in the cancer and/or reduce the volume or size of a tumor comprising the cancer cells.

70. The method of any one of claims 61-69, wherein the at least one composition increases the amount and/or activity of CD8+ T cells infiltrating a tumor comprising the cancer cells.

71. The method of any one of claims 61-70, wherein the at least one composition a) increases the amount and/or activity of M1 macrophages infiltrating a tumor comprising the cancer cells and/or b) decreases the amount and/or activity of M2 macrophages infiltrating a tumor comprising the cancer cells.

72. The method of any one of claims 36-71, wherein the myeloid-derived cells contacted with the at least one composition have a modulated inflammatory phenotype exhibiting one or more of the following:
a) decreased expression of CCR2 and/or CSF1R receptors by monocytes and/or macrophages;
b) increased expression of cluster of differentiation 80 (CD80), CD86, WICK MHCI, interleukin 1-beta (IL-1β), IL-6, CCL3, CCL4, CXCL10, CXCL9, GM-CSF and/or tumor necrosis factor alpha (TNF-α) by monocytes and/or macrophages;
c) decreased expression of CD206, CD163, CD16, CD53, VSIG4, PSGL-1, TGFb and/or IL-10 by monocytes and/or macrophages;
d) increased secretion of at least one cytokine or chemokine selected from the group consisting of IL-1β, TNF-α, IL-12, IL-18, GM-CSF, CCL3, CCL4, and IL-2 by monocytes and/or macrophages;
e) increased ratio of expression of IL-1β, IL-6, and/or TNF-α to expression of IL-10 by monocytes and/or macrophages;
f) increased CD8+ cytotoxic T cell activation;
g) increased recruitment of CD8+ cytotoxic T cell activation;
h) increased CD4+ helper T cell activity;
i) increased recruitment of CD4+ helper T cell activity;
j) increased NK cell activity;
k) increased recruitment of NK cells;
l) increased neutrophil activity;
m) increased macrophage activity; and/or
n) increased spindle-shaped morphology, flatness of appearance, and/or number of dendrites, as assessed by microscopy.

73. The method of any one of claims 36-72, wherein the myeloid-derived cell is a macrophage, a monocyte, a circulating bone marrow derived monocyte, a tissue resident macrophage, a macrophage associated with a clinical condition, a Type 1 macrophage, a M1 macrophage, a Type 2 macrophage, a M2 macrophage, a M2c macrophage, a M2d macrophage, and/or a tumor-associated macrophages (TAM).

74. The method of any one of claims 36-73, wherein the cancer is selected from the group consisting of mesothelioma, kidney renal clear cell carcinoma, glioblastoma, lung adenocarcinoma, lung squamous cell carcinoma, pancreatic adenocarcinoma, breast invasive carcinoma, acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous, cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, and uveal melanoma.

75. The method of any one of claims 36-74, wherein the myeloid-derived cells are comprised within a human tumor model, an animal model of cancer, and/or a thyglycollate peritonitis model.

76. The method of any one of claims 36-75, wherein the subject is a mammal.

77. The method of claim 76, wherein the mammal is a human.

78. The method of claim 77, wherein the human is afflicted with a cancer.


SEQUENCE LISTING (1050 sequences)

SEQ ID NO: 1 - DNA, 2689 bp, Homo sapiens
tttattctctggaacatgaaacattctgttgtgctcatatcatgcaaattatcactagtaggagagcagagagtggaaatgttccaggtataaagacccacaagataaagaagctcagagtcgttagaaacaggagcagatgtacagggtttgcctgactcacactcaaggttgcataagcaagatttcaaaattaatcctattctggagacctcaacccaatgtacaatgttcctgactggaaaagaagaactatatttttctgattttttttttcaaatctttaccattagttgccctgtatctccgccttcactttctgcaggaaactttatttcctacttctgcatgccaagtttctacctctagatctgtttggttcagttgctgagaagcctgacataccaggactgcctgagacaagccacaagctgaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataatattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcctagagacagaaatgacagatctctgctttggaaatcacacgtctggcttcacagatgtgtgattcacagtgtgaatcttggtgtctacgttaccaggcaggaaggctgagaggagagagactccagctgggttggaaaacagtattttccaaactaccttccagttcctcatttttgaatacaggcatagagttcagactttttttaaatagtaaaaataaaattaaagctgaaaactgcaacttgtaaatgtggtaaagagttagtttgagttactatcatgtcaaacgtgaaaatgctgtattagtcacagagataattctagctttgagcttaagaattttgagcaggtggtatgtttgggagactgctgagtcaacccaatagttgttgattggcaggagttggaagtgtgtgatctgtgggcacattagcctatgtgcatgcagcatctaagtaatgatgtcgtttgaatcacagtatacgctccatcgctgtcatctcagctggatctccattctctcaggcttgctgccaaaagccttttgtgttttgttttgtatcattatgaagtcatgcgtttaatcacattcgagtgtttcagtgcttcgcagatgtccttgatgctcatattgttccctattttgccagtgggaactcctaaatcaagttggcttctaatcaaagcttttaaaccctattggtaaagaatggaaggtggagaagctccctgaagtaagcaaagactttcctcttagtcgagccaagttaagaatgttcttatgttgcccagtgtgtttctgatctgatgcaagcaagaaacactgggcttctagaaccaggcaacttgggaactagactcccaagctggactatggctctactttcaggccacatggctaaagaaggtttcagaaagaagtggggacagagcagaactttcaccttcatatatttgtatgatcctaatgaatgcataaaatgttaagttgatggtgatgaaatgtaaatactgtttttaacaactatgatttggaaaataaatcaatgctataactatgttgaaaaaaaaaaaaaaaaaa

SEQ ID NO: 2 - DNA, 4006 bp, Homo sapiens
gaagggcagacagagtgtccaaaagcgtgagagcacgaagtgaggagaaggtggagaagagagaagaggaagaggaagaggaagagaggaagcggagggaactgcggccaggctaaaaggggaagaagaggatcagcccaaggaggaggaagaggaaaacaagacaaacagccagtgcagaggagaggaacgtgtgtccagtgtcccgatccctgcggagctagtagctgagagctctgtgccctgggcaccttgcagccctgcacctgcctgccacttccccaccgaggccatgggcccaggagttctgctgctcctgctggtggccacagcttggcatggtcagggaatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacaccaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccagtggtggtcgcctgcatgtccatcatggccttgctgctgctgctgctcctgctgctattgtacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaaggctgaggccatgctgggacccagcctgagccccggccaggaccccgagggaggcgtcgactataagaacatccacctcgagaagaaatatgtccgcagggacagtggcttctccagccagggtgtggacacctatgtggagatgaggcctgtctccacttcttcaaatgactccttctctgagcaagacctggacaaggaggatggacggcccctggagctccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactataccaatctgccgagcagcagcagaagcggtggcagcggcagcagcagcagtgagctggaggaggagagctctagtgagcacctgacctgctgcgagcaaggggatatcgcccagcccttgctgcagcccaacaactatcagttctgctgaggagttgacgacagggagtaccactctcccctcccacaaacttcaactcctccatggatggggcgacacggggagaacatacaaactctgccttcggtcatttcactcaacagctcggcccagctctgaaacttgggaaggtgagggattcaggggaggtcagaggatcccacttcctgagcatgggccatcactgccagtcaggggctgggggctgagccctcacccccccctcccctactgttctcatggtgttggcctcgtgtttgctatgccaactagtagaaccttctttcctaatccccttatcttcatggaaatggactgactttatgcctatgaagtccccaggagctacactgatactgagaaaaccaggctctttggggctagacagactggcagagagtgagatctccctctctgagaggagcagcagatgctcacagaccacactcagctcaggccccttggagcaggatggctcctctaagaatctcacaggacctcttagtctctgccctatacgccgccttcactccacagcctcacccctcccacccccatactggtactgctgtaatgagccaagtggcagctaaaagttgggggtgttctgcccagtcccgtcattctgggctagaaggcaggggaccttggcatgtggctggccacaccaagcaggaagcacaaactcccccaagctgactcatcctaactaacagtcacgccgtgggatgtctctgtccacattaaactaacagcattaatgcagtcaaaaaaaaaaaaaaaaaa

SEQ ID NO: 3 - DNA, 2335 bp, Homo sapiens
tttattctctggaacatgaaacattctgttgtgctcatatcatgcaaattatcactagtaggagagcagagagtggaaatgttccaggtataaagacccacaagataaagaagctcagagtcgttagaaacaggagcagatgtacagggtttgcctgactcacactcaaggttgcataagcaagatttcaaaattaatcctattctggagacctcaacccaatgtacaatgttcctgactggaaaagaagaactatatttttctgattttttttttcaaatctttaccattagttgccctgtatctccgccttcactttctgcaggaaactttatttcctacttctgcatgccaagtttctacctctagatctgtttggttcagttgctgagaagcctgacataccaggactgcctgagacaagccacaagctgaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataatattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaaggtatctctcggtgttcttccgaaagcacatcaccaagcgcttctgcaaacaatgtccagttttctacagggagacagtggatggagtgacttcaacaaacacgccttccactggggagcaggaagtctcggctggtttataaaacgaggagcagtttgattgttgtttataaagggagataacaatctgtatataacaacaaacttcaagggtttgttgaacaatagaaacctgtaaagcaggtgcccaggaacctcagggctgtgtgtactaatacagactatgtcacccaatgcatatccaacatgtgctcagggaataatccagaaaaactgtgggtagagactttgactctccagaaagctcatctcagctcctgaaaaatgcctcattaccttgtgctaatcctctttttctagtcttcataatttcttcactcaatctctgattctgtcaatgtcttgaaatcaagggccagctggaggtgaagaagagaatgtgacaggcacagatgaatgggagtgagggatagtggggtcagggctgagaggagaaggagggagacatgagcatggctgagcctggacaaagacaaaggtgagcaaagggctcacgcattcagccaggagatgatactggtccttagccccatctgccacgtgtatttaaccttgaagggttcaccaggtcagggagagtttgggaactgcaataacctgggagttttggtggagtccgatgattctcttttgcataagtgcatgacatatttttgctttattacagtttatctatggcacccatgcaccttacatttgaaatctatgaaatatcatgctccattgttcagatgcttcttaggccacatccccctgtctaaaaattcagaaaatttttgtttataaaaga

SEQ ID NO: 4 - DNA, 3986 bp, Homo sapiens
ccctgccccagactgcgacccctccctcttgggttcaaggctttgttttcttcttaaagacccaagatttccaaactctgtggttgccttgcctagctaaaaggggaagaagaggatcagcccaaggaggaggaagaggaaaacaagacaaacagccagtgcagaggagaggaacgtgtgtccagtgtcccgatccctgcggagctagtagctgagagctctgtgccctgggcaccttgcagccctgcacctgcctgccacttccccaccgaggccatgggcccaggagttctgctgctcctgctggtggccacagcttggcatggtcagggaatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacaccaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccagtggtggtcgcctgcatgtccatcatggccttgctgctgctgctgctcctgctgctattgtacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaaggctgaggccatgctgggacccagcctgagccccggccaggaccccgagggaggcgtcgactataagaacatccacctcgagaagaaatatgtccgcagggacagtggcttctccagccagggtgtggacacctatgtggagatgaggcctgtctccacttcttcaaatgactccttctctgagcaagacctggacaaggaggatggacggcccctggagctccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactataccaatctgccgagcagcagcagaagcggtggcagcggcagcagcagcagtgagctggaggaggagagctctagtgagcacctgacctgctgcgagcaaggggatatcgcccagcccttgctgcagcccaacaactatcagttctgctgaggagttgacgacagggagtaccactctcccctcccacaaacttcaactcctccatggatggggcgacacggggagaacatacaaactctgccttcggtcatttcactcaacagctcggcccagctctgaaacttgggaaggtgagggattcaggggaggtcagaggatcccacttcctgagcatgggccatcactgccagtcaggggctgggggctgagccctcacccccccctcccctactgttctcatggtgttggcctcgtgtttgctatgccaactagtagaaccttctttcctaatccccttatcttcatggaaatggactgactttatgcctatgaagtccccaggagctacactgatactgagaaaaccaggctctttggggctagacagactggcagagagtgagatctccctctctgagaggagcagcagatgctcacagaccacactcagctcaggccccttggagcaggatggctcctctaagaatctcacaggacctcttagtctctgccctatacgccgccttcactccacagcctcacccctcccacccccatactggtactgctgtaatgagccaagtggcagctaaaagttgggggtgttctgcccagtcccgtcattctgggctagaaggcaggggaccttggcatgtggctggccacaccaagcaggaagcacaaactcccccaagctgactcatcctaactaacagtcacgccgtgggatgtctctgtccacattaaactaacagcattaatgcagtcaaaaaaaaaaaaaa

SEQ ID NO: 5 - DNA, 4087 bp, Homo sapiens
ctcttttgcctgccatcatgttggatgtgattctgctcctcctttgccttccactatgattctgaggcctcctcagccatgctgaactgtttacctgttctggatgtttcatatagatggagtcgtatgacattttgctactggcttcattgacttaacacagtgttttcaaggttcatccacagtgtagcagctaaaaggggaagaagaggatcagcccaaggaggaggaagaggaaaacaagacaaacagccagtgcagaggagaggaacgtgtgtccagtgtcccgatccctgcggagctagtagctgagagctctgtgccctgggcaccttgcagccctgcacctgcctgccacttccccaccgaggccatgggcccaggagttctgctgctcctgctggtggccacagcttggcatggtcagggaatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacaccaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccagtggtggtcgcctgcatgtccatcatggccttgctgctgctgctgctcctgctgctattgtacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaaggctgaggccatgctgggacccagcctgagccccggccaggaccccgagggaggcgtcgactataagaacatccacctcgagaagaaatatgtccgcagggacagtggcttctccagccagggtgtggacacctatgtggagatgaggcctgtctccacttcttcaaatgactccttctctgagcaagacctggacaaggaggatggacggcccctggagctccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactataccaatctgccgagcagcagcagaagcggtggcagcggcagcagcagcagtgagctggaggaggagagctctagtgagcacctgacctgctgcgagcaaggggatatcgcccagcccttgctgcagcccaacaactatcagttctgctgaggagttgacgacagggagtaccactctcccctcccacaaacttcaactcctccatggatggggcgacacggggagaacatacaaactctgccttcggtcatttcactcaacagctcggcccagctctgaaacttgggaaggtgagggattcaggggaggtcagaggatcccacttcctgagcatgggccatcactgccagtcaggggctgggggctgagccctcacccccccctcccctactgttctcatggtgttggcctcgtgtttgctatgccaactagtagaaccttctttcctaatccccttatcttcatggaaatggactgactttatgcctatgaagtccccaggagctacactgatactgagaaaaccaggctctttggggctagacagactggcagagagtgagatctccctctctgagaggagcagcagatgctcacagaccacactcagctcaggccccttggagcaggatggctcctctaagaatctcacaggacctcttagtctctgccctatacgccgccttcactccacagcctcacccctcccacccccatactggtactgctgtaatgagccaagtggcagctaaaagttgggggtgttctgcccagtcccgtcattctgggctagaaggcaggggaccttggcatgtggctggccacaccaagcaggaagcacaaactcccccaagctgactcatcctaactaacagtcacgccgtgggatgtctctgtccacattaaactaacagcattaatgcagtcaaaaaaaaaaaaaaaaaa

SEQ ID NO: 6 - RNA, 19 nt, Artificial Sequence
  Description of Artificial Sequence Synthetic oligonucleotide
cauuuccccaguacaucca

SEQ ID NO: 7 - RNA, 19 nt, Artificial Sequence
  Description of Artificial Sequence Synthetic oligonucleotide
auuuccccaguacauccac

SEQ ID NO: 8 - RNA, 19 nt, Artificial Sequence
  Description of Artificial Sequence Synthetic oligonucleotide
augcuguccacaucucguu

SEQ ID NO: 9 - RNA, 19 nt, Artificial Sequence
  Description of Artificial Sequence Synthetic oligonucleotide
ugcuguccacaucucguuc

SEQ ID NO: 10 - RNA, 19 nt, Artificial Sequence
  Description of Artificial Sequence Synthetic oligonucleotide
gcuguccacaucucguucu

SEQ ID NO: 11 - RNA, 19 nt, Artificial Sequence
  Description of Artificial Sequence Synthetic oligonucleotide
cuguccacaucucguucuc

SEQ ID NO: 12 - RNA, 19 nt, Artificial Sequence
  Description of Artificial Sequence Synthetic oligonucleotide
guccacaucucguucucgg

SEQ ID NO: 13 - RNA, 19 nt, Artificial Sequence
  Description of Artificial Sequence Synthetic oligonucleotide
agagcggugaagaagucac

SEQ ID NO: 14 - RNA, 19 nt, Artificial Sequence
  Description of Artificial Sequence Synthetic oligonucleotide
cggugaagaagucaccacc

SEQ ID NO: 15 - RNA, 19 nt, Artificial Sequence
  Description of Artificial Sequence Synthetic oligonucleotide
aagucaccaccuuuuuuga

SEQ ID NO: 16 - RNA, 19 nt, Artificial Sequence
  Description of Artificial Sequence Synthetic oligonucleotide
ucaccaccuuuuuugauua

SEQ ID NO: 17 - RNA, 19 nt, Artificial Sequence
  Description of Artificial Sequence Synthetic oligonucleotide
caccaccuuuuuugauuau

SEQ ID NO: 18 - RNA, 19 nt, Artificial Sequence
  Description of Artificial Sequence Synthetic oligonucleotide
accaccuuuuuugauuaug

SEQ ID NO: 19 - RNA, 19 nt, Artificial Sequence
  Description of Artificial Sequence Synthetic oligonucleotide
gugcucccugucauaaauu

SEQ ID NO: 20 - RNA, 19 nt, Artificial Sequence
  Description of Artificial Sequence Synthetic oligonucleotide
ugcucccugucauaaauuu

SEQ ID NO: 21 - RNA, 19 nt, Artificial Sequence
  Description of Artificial Sequence Synthetic oligonucleotide
cucccugucauaaauuuga

SEQ ID NO: 22 - RNA, 19 nt, Artificial Sequence
  Description of Artificial Sequence Synthetic oligonucleotide
ccugucauaaauuugacgu

SEQ ID NO: 23 - RNA, 19 nt, Artificial Sequence
  Description of Artificial Sequence Synthetic oligonucleotide
ugucauaaauuugacguga

SEQ ID NO: 24 - RNA, 19 nt, Artificial Sequence
  Description of Artificial Sequence Synthetic oligonucleotide
gucauaaauuugacgugaa

SEQ ID NO: 25 - RNA, 19 nt, Artificial Sequence
  Description of Artificial Sequence Synthetic oligonucleotide
ucauaaauuugacgugaag

SEQ ID NO: 26 - RNA, 19 nt, Artificial Sequence
  Description of Artificial Sequence Synthetic oligonucleotide
auaaauuugacgugaagca

SEQ ID NO: 27 - RNA, 19 nt, Artificial Sequence
  Description of Artificial Sequence Synthetic oligonucleotide
aauuugacgugaagcaaau

SEQ ID NO: 28 - RNA, 19 nt, Artificial Sequence
  Description of Artificial Sequence Synthetic oligonucleotide
auuugacgugaagcaaauu

SEQ ID NO: 29 - RNA, 19 nt, Artificial Sequence
  Description of Artificial Sequence Synthetic oligonucleotide
cacucugcugcaaaugagu

SEQ ID NO: 30 - RNA, 19 nt, Artificial Sequence
  Description of Artificial Sequence Synthetic oligonucleotide
agugggucuuugggaaugc

SEQ ID NO: 31 - RNA, 19 nt, Artificial Sequence
  Description of Artificial Sequence Synthetic oligonucleotide
gugggucuuugggaaugca

SEQ ID NO: 32 - RNA, 19 nt, Artificial Sequence
  Description of Artificial Sequence Synthetic oligonucleotide
gggucuuugggaaugcaau

SEQ ID NO: 33 - RNA, 19 nt, Artificial Sequence
  Description of Artificial Sequence Synthetic oligonucleotide
uugggaaugcaaugugcaa

SEQ ID NO: 34 - RNA, 19 nt, Artificial Sequence
  Description of Artificial Sequence Synthetic oligonucleotide
gugcaaauuauucacaggg

SEQ ID NO: 35 - RNA, 19 nt, Artificial Sequence
  Description of Artificial Sequence Synthetic oligonucleotide
cacagggcuguaucacauc

SEQ ID NO: 36 - RNA, 19 nt, Artificial Sequence
  Description of Artificial Sequence Synthetic oligonucleotide
cgguuauuuuggcggaauc

SEQ ID NO: 37 - RNA, 19 nt, Artificial Sequence
  Description of Artificial Sequence Synthetic oligonucleotide
guuauuuuggcggaaucuu

SEQ ID NO: 38 - RNA, 19 nt, Artificial Sequence
  Description of Artificial Sequence Synthetic oligonucleotide
uuauuuuggcggaaucuuc

SEQ ID NO: 39 - RNA, 19 nt, Artificial Sequence
  Description of Artificial Sequence Synthetic oligonucleotide
uauuuuggcggaaucuucu

SEQ ID NO: 40 - RNA, 19 nt, Artificial Sequence
  Description of Artificial Sequence Synthetic oligonucleotide
auuuuggcggaaucuucuu

SEQ ID NO: 41 - RNA, 19 nt, Artificial Sequence
  Description of Artificial Sequence Synthetic oligonucleotide
uuuuggcggaaucuucuuc

SEQ ID NO: 42 - RNA, 19 nt, Artificial Sequence
  Description of Artificial Sequence Synthetic oligonucleotide
uuuggcggaaucuucuuca

SEQ ID NO: 43 - RNA, 19 nt, Artificial Sequence
  Description of Artificial Sequence Synthetic oligonucleotide
aagugugaucaccugguug

SEQ ID NO: 44 - RNA, 19 nt, Artificial Sequence
  Description of Artificial Sequence Synthetic oligonucleotide
ugugaucaccugguuggug

SEQ ID NO: 45 - RNA, 19 nt, Artificial Sequence
  Description of Artificial Sequence Synthetic oligonucleotide
gugaucaccugguuggugg

SEQ ID NO: 46 - RNA, 19 nt, Artificial Sequence
  Description of Artificial Sequence Synthetic oligonucleotide
ugaucaccugguugguggc

SEQ ID NO: 47 - RNA, 19 nt, Artificial Sequence
  Description of Artificial Sequence Synthetic oligonucleotide
caccugguugguggcugug

SEQ ID NO: 48 - RNA, 19 nt, Artificial Sequence
  Description of Artificial Sequence Synthetic oligonucleotide
aggaaucaucuuuacuaaa

SEQ ID NO: 49 - RNA, 19 nt, Artificial Sequence
  Description of Artificial Sequence Synthetic oligonucleotide
uggcccuuauuuuccacga

SEQ ID NO: 50 - RNA, 19 nt, Artificial Sequence
  Description of Artificial Sequence Synthetic oligonucleotide
ggcccuuauuuuccacgag

SEQ ID NO: 51 - RNA, 19 nt, Artificial Sequence
  Description of Artificial Sequence Synthetic oligonucleotide
gcccuuauuuuccacgagg

SEQ ID NO: 52 - RNA, 19 nt, Artificial Sequence
  Description of Artificial Sequence Synthetic oligonucleotide
cuuauuuuccacgaggaug

SEQ ID NO: 53 - RNA, 19 nt, Artificial Sequence
  Description of Artificial Sequence Synthetic oligonucleotide
uuauuuuccacgaggaugg

SEQ ID NO: 54 - RNA, 19 nt, Artificial Sequence
  Description of Artificial Sequence Synthetic oligonucleotide
uauuuuccacgaggaugga

SEQ ID NO: 55 - RNA, 19 nt, Artificial Sequence
  Description of Artificial Sequence Synthetic oligonucleotide
uuuccacgaggauggaaua

SEQ ID NO: 56 - RNA, 19 nt, Artificial Sequence
  Description of Artificial Sequence Synthetic oligonucleotide
uuccacgaggauggaauaa

SEQ ID NO: 57 - RNA, 19 nt, Artificial Sequence
  Description of Artificial Sequence Synthetic oligonucleotide
uccacgaggauggaauaau

SEQ ID NO: 58 - RNA, 19 nt, Artificial Sequence
  Description of Artificial Sequence Synthetic oligonucleotide
cacgaggauggaauaauuu

SEQ ID NO: 59 - RNA, 19 nt, Artificial Sequence
  Description of Artificial Sequence Synthetic oligonucleotide
aauuuccacacaauaauga

SEQ ID NO: 60 - RNA, 19 nt, Artificial Sequence
  Description of Artificial Sequence Synthetic oligonucleotide
uuuccacacaauaaugagg

SEQ ID NO: 61 - RNA, 19 nt, Artificial Sequence
  Description of Artificial Sequence Synthetic oligonucleotide
cacacaauaaugaggaaca

SEQ ID NO: 62 - RNA, 19 nt, Artificial Sequence
  Description of Artificial Sequence Synthetic oligonucleotide
aacauuuuggggcuggucc

SEQ ID NO: 63 - RNA, 19 nt, Artificial Sequence
  Description of Artificial Sequence Synthetic oligonucleotide
aauucuucggccugaguaa

SEQ ID NO: 64 - RNA, 19 nt, Artificial Sequence
  Description of Artificial Sequence Synthetic oligonucleotide
auucuucggccugaguaac

SEQ ID NO: 65 - RNA, 19 nt, Artificial Sequence
  Description of Artificial Sequence Synthetic oligonucleotide
uucuucggccugaguaacu

SEQ ID NO: 66 - RNA, 19 nt, Artificial Sequence
  Description of Artificial Sequence Synthetic oligonucleotide
uucggccugaguaacugug

SEQ ID NO: 67 - RNA, 19 nt, Artificial Sequence
  Description of Artificial Sequence Synthetic oligonucleotide
ugaguaacugugaaagcac

SEQ ID NO: 68 - RNA, 19 nt, Artificial Sequence
  Description of Artificial Sequence Synthetic oligonucleotide
uggauguacuggggaaaug

SEQ ID NO: 69 - RNA, 19 nt, Artificial Sequence
  Description of Artificial Sequence Synthetic oligonucleotide
guggauguacuggggaaau

SEQ ID NO: 70 - RNA, 19 nt, Artificial Sequence
  Description of Artificial Sequence Synthetic oligonucleotide
aacgagauguggacagcau

SEQ ID NO: 71 - RNA, 19 nt, Artificial Sequence
  Description of Artificial Sequence Synthetic oligonucleotide
gaacgagauguggacagca

SEQ ID NO: 72 - RNA, 19 nt, Artificial Sequence
  Description of Artificial Sequence Synthetic oligonucleotide
agaacgagauguggacagc

SEQ ID NO: 73 - RNA, 19 nt, Artificial Sequence
  Description of Artificial Sequence Synthetic oligonucleotide
gagaacgagauguggacag

SEQ ID NO: 74 - RNA, 19 nt, Artificial Sequence
  Description of Artificial Sequence Synthetic oligonucleotide
ccgagaacgagauguggac

SEQ ID NO: 75 - RNA, 19 nt, Artificial Sequence
  Description of Artificial Sequence Synthetic oligonucleotide
gugacuucuucaccgcucu

SEQ ID NO: 76 - RNA, 19 nt, Artificial Sequence
  Description of Artificial Sequence Synthetic oligonucleotide
gguggugacuucuucaccg

SEQ ID NO: 77 - RNA, 19 nt, Artificial Sequence
  Description of Artificial Sequence Synthetic oligonucleotide
ucaaaaaagguggugacuu

SEQ ID NO: 78 - RNA, 19 nt, Artificial Sequence
  Description of Artificial Sequence Synthetic oligonucleotide
uaaucaaaaaaggugguga

SEQ ID NO: 79 - RNA, 19 nt, Artificial Sequence
  Description of Artificial Sequence Synthetic oligonucleotide
auaaucaaaaaagguggug

SEQ ID NO: 80 - RNA, 19 nt, Artificial Sequence
  Description of Artificial Sequence Synthetic oligonucleotide
cauaaucaaaaaagguggu

SEQ ID NO: 81 - RNA, 19 nt, Artificial Sequence
  Description of Artificial Sequence Synthetic oligonucleotide
aauuuaugacagggagcac

SEQ ID NO: 82 - RNA, 19 nt, Artificial Sequence
  Description of Artificial Sequence Synthetic oligonucleotide
aaauuuaugacagggagca

SEQ ID NO: 83 - RNA, 19 nt, Artificial Sequence
  Description of Artificial Sequence Synthetic oligonucleotide
ucaaauuuaugacagggag

SEQ ID NO: 84 - RNA, 19 nt, Artificial Sequence
  Description of Artificial Sequence Synthetic oligonucleotide
acgucaaauuuaugacagg

SEQ ID NO: 85 - RNA, 19 nt, Artificial Sequence
  Description of Artificial Sequence Synthetic oligonucleotide
ucacgucaaauuuaugaca

SEQ ID NO: 86 - RNA, 19 nt, Artificial Sequence
  Description of Artificial Sequence Synthetic oligonucleotide
uucacgucaaauuuaugac

SEQ ID NO: 87 - RNA, 19 nt, Artificial Sequence
  Description of Artificial Sequence Synthetic oligonucleotide
cuucacgucaaauuuauga

SEQ ID NO: 88 - RNA, 19 nt, Artificial Sequence
  Description of Artificial Sequence Synthetic oligonucleotide
ugcuucacgucaaauuuau

SEQ ID NO: 89 - RNA, 19 nt, Artificial Sequence
  Description of Artificial Sequence Synthetic oligonucleotide
auuugcuucacgucaaauu

SEQ ID NO: 90 - RNA, 19 nt, Artificial Sequence
  Description of Artificial Sequence Synthetic oligonucleotide
aauuugcuucacgucaaau

SEQ ID NO: 91 - RNA, 19 nt, Artificial Sequence
  Description of Artificial Sequence Synthetic oligonucleotide
acucauuugcagcagagug

SEQ ID NO: 92 - RNA, 19 nt, Artificial Sequence
  Description of Artificial Sequence Synthetic oligonucleotide
gcauucccaaagacccacu

SEQ ID NO: 93 - RNA, 19 nt, Artificial Sequence
  Description of Artificial Sequence Synthetic oligonucleotide
ugcauucccaaagacccac

SEQ ID NO: 94 - RNA, 19 nt, Artificial Sequence
  Description of Artificial Sequence Synthetic oligonucleotide
auugcauucccaaagaccc

SEQ ID NO: 95 - RNA, 19 nt, Artificial Sequence
  Description of Artificial Sequence Synthetic oligonucleotide
uugcacauugcauucccaa

SEQ ID NO: 96 - RNA, 19 nt, Artificial Sequence
  Description of Artificial Sequence Synthetic oligonucleotide
cccugugaauaauuugcac

SEQ ID NO: 97 - RNA, 19 nt, Artificial Sequence
  Description of Artificial Sequence Synthetic oligonucleotide
gaugugauacagcccugug

SEQ ID NO: 98 - RNA, 19 nt, Artificial Sequence
  Description of Artificial Sequence Synthetic oligonucleotide
gauuccgccaaaauaaccg

SEQ ID NO: 99 - RNA, 19 nt, Artificial Sequence
  Description of Artificial Sequence Synthetic oligonucleotide
aagauuccgccaaaauaac

SEQ ID NO: 100 - RNA, 19 nt, Artificial Sequence
  Description of Artificial Sequence Synthetic oligonucleotide
gaagauuccgccaaaauaa

SEQ ID NO: 101 - RNA, 19 nt, Artificial Sequence
  Description of Artificial Sequence Synthetic oligonucleotide
agaagauuccgccaaaaua

SEQ ID NO: 102 - RNA, 19 nt, Artificial Sequence
  Description of Artificial Sequence Synthetic oligonucleotide
aagaagauuccgccaaaau

SEQ ID NO: 103 - RNA, 19 nt, Artificial Sequence
  Description of Artificial Sequence Synthetic oligonucleotide
gaagaagauuccgccaaaa

SEQ ID NO: 104 - RNA, 19 nt, Artificial Sequence
  Description of Artificial Sequence Synthetic oligonucleotide
ugaagaagauuccgccaaa

SEQ ID NO: 105 - RNA, 19 nt, Artificial Sequence
  Description of Artificial Sequence Synthetic oligonucleotide
caaccaggugaucacacuu

SEQ ID NO: 106 - RNA, 19 nt, Artificial Sequence
  Description of Artificial Sequence Synthetic oligonucleotide
caccaaccaggugaucaca

SEQ ID NO: 107 - RNA, 19 nt, Artificial Sequence
  Description of Artificial Sequence Synthetic oligonucleotide
ccaccaaccaggugaucac

SEQ ID NO: 108 - RNA, 19 nt, Artificial Sequence
  Description of Artificial Sequence Synthetic oligonucleotide
gccaccaaccaggugauca

SEQ ID NO: 109 - RNA, 19 nt, Artificial Sequence
  Description of Artificial Sequence Synthetic oligonucleotide
cacagccaccaaccaggug

SEQ ID NO: 110 - RNA, 19 nt, Artificial Sequence
  Description of Artificial Sequence Synthetic oligonucleotide
uuuaguaaagaugauuccu

SEQ ID NO: 111 - RNA, 19 nt, Artificial Sequence
  Description of Artificial Sequence Synthetic oligonucleotide
ucguggaaaauaagggcca

SEQ ID NO: 112 - RNA, 19 nt, Artificial Sequence
  Description of Artificial Sequence Synthetic oligonucleotide
cucguggaaaauaagggcc

SEQ ID NO: 113 - RNA, 19 nt, Artificial Sequence
  Description of Artificial Sequence Synthetic oligonucleotide
ccucguggaaaauaagggc

SEQ ID NO: 114 - RNA, 19 nt, Artificial Sequence
  Description of Artificial Sequence Synthetic oligonucleotide
cauccucguggaaaauaag

SEQ ID NO: 115 - RNA, 19 nt, Artificial Sequence
  Description of Artificial Sequence Synthetic oligonucleotide
ccauccucguggaaaauaa

SEQ ID NO: 116 - RNA, 19 nt, Artificial Sequence
  Description of Artificial Sequence Synthetic oligonucleotide
uccauccucguggaaaaua

SEQ ID NO: 117 - RNA, 19 nt, Artificial Sequence
  Description of Artificial Sequence Synthetic oligonucleotide
uauuccauccucguggaaa

SEQ ID NO: 118 - RNA, 19 nt, Artificial Sequence
  Description of Artificial Sequence Synthetic oligonucleotide
uuauuccauccucguggaa

SEQ ID NO: 119 - RNA, 19 nt, Artificial Sequence
  Description of Artificial Sequence Synthetic oligonucleotide
auuauuccauccucgugga

SEQ ID NO: 120 - RNA, 19 nt, Artificial Sequence
  Description of Artificial Sequence Synthetic oligonucleotide
aaauuauuccauccucgug

SEQ ID NO: 121 - RNA, 19 nt, Artificial Sequence
  Description of Artificial Sequence Synthetic oligonucleotide
ucauuauuguguggaaauu

SEQ ID NO: 122 - RNA, 19 nt, Artificial Sequence
  Description of Artificial Sequence Synthetic oligonucleotide
ccucauuauuguguggaaa

SEQ ID NO: 123 - RNA, 19 nt, Artificial Sequence
  Description of Artificial Sequence Synthetic oligonucleotide
uguuccucauuauugugug

SEQ ID NO: 124 - RNA, 19 nt, Artificial Sequence
  Description of Artificial Sequence Synthetic oligonucleotide
ggaccagccccaaaauguu

SEQ ID NO: 125 - RNA, 19 nt, Artificial Sequence
  Description of Artificial Sequence Synthetic oligonucleotide
uuacucaggccgaagaauu

SEQ ID NO: 126 - RNA, 19 nt, Artificial Sequence
  Description of Artificial Sequence Synthetic oligonucleotide
guuacucaggccgaagaau

SEQ ID NO: 127 - RNA, 19 nt, Artificial Sequence
  Description of Artificial Sequence Synthetic oligonucleotide
aguuacucaggccgaagaa

SEQ ID NO: 128 - RNA, 19 nt, Artificial Sequence
  Description of Artificial Sequence Synthetic oligonucleotide
cacaguuacucaggccgaa

SEQ ID NO: 129 - RNA, 19 nt, Artificial Sequence
  Description of Artificial Sequence Synthetic oligonucleotide
gugcuuucacaguuacuca

SEQ ID NO: 130 - RNA, 19 nt, Artificial Sequence
  Description of Artificial Sequence Synthetic oligonucleotide
gggaaucccagugauagag

SEQ ID NO: 131 - RNA, 19 nt, Artificial Sequence
  Description of Artificial Sequence Synthetic oligonucleotide
uugcgaugugugggcaaug

SEQ ID NO: 132 - RNA, 19 nt, Artificial Sequence
  Description of Artificial Sequence Synthetic oligonucleotide
gcgaugugugggcaauggc

SEQ ID NO: 133 - RNA, 19 nt, Artificial Sequence
  Description of Artificial Sequence Synthetic oligonucleotide
caacgcuaccuuccaaaac

SEQ ID NO: 134 - RNA, 19 nt, Artificial Sequence
  Description of Artificial Sequence Synthetic oligonucleotide
acgcuaccuuccaaaacac

SEQ ID NO: 135 - RNA, 19 nt, Artificial Sequence
  Description of Artificial Sequence Synthetic oligonucleotide
cgcuaccuuccaaaacacg

SEQ ID NO: 136 - RNA, 19 nt, Artificial Sequence
  Description of Artificial Sequence Synthetic oligonucleotide
ugcccggcccuggaacgug

SEQ ID NO: 137 - RNA, 19 nt, Artificial Sequence
  Description of Artificial Sequence Synthetic oligonucleotide
ugcucacagacccggugcu

SEQ ID NO: 138 - RNA, 19 nt, Artificial Sequence
  Description of Artificial Sequence Synthetic oligonucleotide
aggcgucucgcuggugcgu

SEQ ID NO: 139 - RNA, 19 nt, Artificial Sequence
  Description of Artificial Sequence Synthetic oligonucleotide
ucaaugcagugcccugaug

SEQ ID NO: 140 - RNA, 19 nt, Artificial Sequence
  Description of Artificial Sequence Synthetic oligonucleotide
cagcauccggcugaaagug

SEQ ID NO: 141 - RNA, 19 nt, Artificial Sequence
  Description of Artificial Sequence Synthetic oligonucleotide
cagagcuggugcggauucg

SEQ ID NO: 142 - RNA, 19 nt, Artificial Sequence
  Description of Artificial Sequence Synthetic oligonucleotide
gagcuggugcggauucgag

SEQ ID NO: 143 - RNA, 19 nt, Artificial Sequence
  Description of Artificial Sequence Synthetic oligonucleotide
ugcggauucgaggggaggc

SEQ ID NO: 144 - RNA, 19 nt, Artificial Sequence
  Description of Artificial Sequence Synthetic oligonucleotide
cugcccagaucgugugcuc

SEQ ID NO: 145 - RNA, 19 nt, Artificial Sequence
  Description of Artificial Sequence Synthetic oligonucleotide
gcccagaucgugugcucag

SEQ ID NO: 146 - RNA, 19 nt, Artificial Sequence
  Description of Artificial Sequence Synthetic oligonucleotide
cagaucgugugcucagcca

SEQ ID NO: 147 - RNA, 19 nt, Artificial Sequence
  Description of Artificial Sequence Synthetic oligonucleotide
auaaccguuaccaaaaagu

SEQ ID NO: 148 - RNA, 19 nt, Artificial Sequence
  Description of Artificial Sequence Synthetic oligonucleotide
aaccguuaccaaaaagucc

SEQ ID NO: 149 - RNA, 19 nt, Artificial Sequence
  Description of Artificial Sequence Synthetic oligonucleotide
accguuaccaaaaaguccu

SEQ ID NO: 150 - RNA, 19 nt, Artificial Sequence
  Description of Artificial Sequence Synthetic oligonucleotide
uuaccaaaaaguccugacc

SEQ ID NO: 151 - RNA, 19 nt, Artificial Sequence
  Description of Artificial Sequence Synthetic oligonucleotide
agguuuuaacuggaccuac

SEQ ID NO: 152 - RNA, 19 nt, Artificial Sequence
  Description of Artificial Sequence Synthetic oligonucleotide
gguuuuaacuggaccuacc

SEQ ID NO: 153 - RNA, 19 nt, Artificial Sequence
  Description of Artificial Sequence Synthetic oligonucleotide
uuuaacuggaccuaccugg

SEQ ID NO: 154 - RNA, 19 nt, Artificial Sequence
  Description of Artificial Sequence Synthetic oligonucleotide
uuaacuggaccuaccuggg

SEQ ID NO: 155 - RNA, 19 nt, Artificial Sequence
  Description of Artificial Sequence Synthetic oligonucleotide
gaccuaccugggacccuuu

SEQ ID NO: 156 - RNA, 19 nt, Artificial Sequence
  Description of Artificial Sequence Synthetic oligonucleotide
ugacguuugagcucacccu

SEQ ID NO: 157 - RNA, 19 nt, Artificial Sequence
  Description of Artificial Sequence Synthetic oligonucleotide
gacguuugagcucacccuu

SEQ ID NO: 158 - RNA, 19 nt, Artificial Sequence
  Description of Artificial Sequence Synthetic oligonucleotide
uugagcucacccuucgaua

SEQ ID NO: 159 - RNA, 19 nt, Artificial Sequence
  Description of Artificial Sequence Synthetic oligonucleotide
agcucacccuucgauaccc

SEQ ID NO: 160 - RNA, 19 nt, Artificial Sequence
  Description of Artificial Sequence Synthetic oligonucleotide
uucgauaccccccagaggu

SEQ ID NO: 161 - RNA, 19 nt, Artificial Sequence
  Description of Artificial Sequence Synthetic oligonucleotide
gauaccccccagagguaag

SEQ ID NO: 162 - RNA, 19 nt, Artificial Sequence
  Description of Artificial Sequence Synthetic oligonucleotide
gagaccuuagagcacaacc

SEQ ID NO: 163 - RNA, 19 nt, Artificial Sequence
  Description of Artificial Sequence Synthetic oligonucleotide
gaccuuagagcacaaccaa

SEQ ID NO: 164 - RNA, 19 nt, Artificial Sequence
  Description of Artificial Sequence Synthetic oligonucleotide
cagcguggggaguggcucc

SEQ ID NO: 165 - RNA, 19 nt, Artificial Sequence
  Description of Artificial Sequence Synthetic oligonucleotide
aucccccggaugaguuccu

SEQ ID NO: 166 - RNA, 19 nt, Artificial Sequence
  Description of Artificial Sequence Synthetic oligonucleotide
cccggaugaguuccucuuc

SEQ ID NO: 167 - RNA, 19 nt, Artificial Sequence
  Description of Artificial Sequence Synthetic oligonucleotide
guggucgccugcaugucca

SEQ ID NO: 168 - RNA, 19 nt, Artificial Sequence
  Description of Artificial Sequence Synthetic oligonucleotide
ugcuauuguacaaguauaa

SEQ ID NO: 169 - RNA, 19 nt, Artificial Sequence
  Description of Artificial Sequence Synthetic oligonucleotide
cagguccgcuggaagauca

SEQ ID NO: 170 - RNA, 19 nt, Artificial Sequence
  Description of Artificial Sequence Synthetic oligonucleotide
gguccgcuggaagaucauc

SEQ ID NO: 171 - RNA, 19 nt, Artificial Sequence
  Description of Artificial Sequence Synthetic oligonucleotide
uccgcuggaagaucaucga

SEQ ID NO: 172 - RNA, 19 nt, Artificial Sequence
  Description of Artificial Sequence Synthetic oligonucleotide
ccgcuggaagaucaucgag

SEQ ID NO: 173 - RNA, 19 nt, Artificial Sequence
  Description of Artificial Sequence Synthetic oligonucleotide
aucaucgagagcuaugagg

SEQ ID NO: 174 - RNA, 19 nt, Artificial Sequence
  Description of Artificial Sequence Synthetic oligonucleotide
caucgagagcuaugagggc

SEQ ID NO: 175 - RNA, 19 nt, Artificial Sequence
  Description of Artificial Sequence Synthetic oligonucleotide
uaugagggcaacaguuaua

SEQ ID NO: 176 - RNA, 19 nt, Artificial Sequence
  Description of Artificial Sequence Synthetic oligonucleotide
agggcaacaguuauacuuu

SEQ ID NO: 177 - RNA, 19 nt, Artificial Sequence
  Description of Artificial Sequence Synthetic oligonucleotide
ggcaacaguuauacuuuca

SEQ ID NO: 178 - RNA, 19 nt, Artificial Sequence
  Description of Artificial Sequence Synthetic oligonucleotide
cacgcagcugccuuacaac

SEQ ID NO: 179 - RNA, 19 nt, Artificial Sequence
  Description of Artificial Sequence Synthetic oligonucleotide
uucccccggaacaaccugc

SEQ ID NO: 180 - RNA, 19 nt, Artificial Sequence
  Description of Artificial Sequence Synthetic oligonucleotide
ccggaacaaccugcaguuu

SEQ ID NO: 181 - RNA, 19 nt, Artificial Sequence
  Description of Artificial Sequence Synthetic oligonucleotide
uuugguaagacccucggag

SEQ ID NO: 182 - RNA, 19 nt, Artificial Sequence
  Description of Artificial Sequence Synthetic oligonucleotide
ugguaagacccucggagcu

SEQ ID NO: 183 - RNA, 19 nt, Artificial Sequence
  Description of Artificial Sequence Synthetic oligonucleotide
ugaaguccacggcccaugc

SEQ ID NO: 184 - RNA, 19 nt, Artificial Sequence
  Description of Artificial Sequence Synthetic oligonucleotide
gaaguccacggcccaugcu

SEQ ID NO: 185 - RNA, 19 nt, Artificial Sequence
  Description of Artificial Sequence Synthetic oligonucleotide
ccuguacuggucaucacgg

SEQ ID NO: 186 - RNA, 19 nt, Artificial Sequence
  Description of Artificial Sequence Synthetic oligonucleotide
cuguacuggucaucacgga

SEQ ID NO: 187 - RNA, 19 nt, Artificial Sequence
  Description of Artificial Sequence Synthetic oligonucleotide
uguacuggucaucacggag

SEQ ID NO: 188 - RNA, 19 nt, Artificial Sequence
  Description of Artificial Sequence Synthetic oligonucleotide
uacuggucaucacggagua

SEQ ID NO: 189 - RNA, 19 nt, Artificial Sequence
  Description of Artificial Sequence Synthetic oligonucleotide
ggucaucacggaguacugu

SEQ ID NO: 190 - RNA, 19 nt, Artificial Sequence
  Description of Artificial Sequence Synthetic oligonucleotide
aucacggaguacuguugcu

SEQ ID NO: 191 - RNA, 19 nt, Artificial Sequence
  Description of Artificial Sequence Synthetic oligonucleotide
ucacggaguacuguugcua

SEQ ID NO: 192 - RNA, 19 nt, Artificial Sequence
  Description of Artificial Sequence Synthetic oligonucleotide
acggaguacuguugcuaug

SEQ ID NO: 193 - RNA, 19 nt, Artificial Sequence
  Description of Artificial Sequence Synthetic oligonucleotide
aguacuguugcuauggcga

SEQ ID NO: 194 - RNA, 19 nt, Artificial Sequence
  Description of Artificial Sequence Synthetic oligonucleotide
uguugcuauggcgaccugc

SEQ ID NO: 195 - RNA, 19 nt, Artificial Sequence
  Description of Artificial Sequence Synthetic oligonucleotide
gcuauggcgaccugcucaa

SEQ ID NO: 196 - RNA, 19 nt, Artificial Sequence
  Description of Artificial Sequence Synthetic oligonucleotide
auggcgaccugcucaacuu

SEQ ID NO: 197 - RNA, 19 nt, Artificial Sequence
  Description of Artificial Sequence Synthetic oligonucleotide
gcucaacuuucugcgaagg

SEQ ID NO: 198 - RNA, 19 nt, Artificial Sequence
  Description of Artificial Sequence Synthetic oligonucleotide
caacuuucugcgaaggaag

SEQ ID NO: 199 - RNA, 19 nt, Artificial Sequence
  Description of Artificial Sequence Synthetic oligonucleotide
uucugcgaaggaaggcuga

SEQ ID NO: 200 - RNA, 19 nt, Artificial Sequence
  Description of Artificial Sequence Synthetic oligonucleotide
caggguguggacaccuaug

SEQ ID NO: 201 - RNA, 19 nt, Artificial Sequence
  Description of Artificial Sequence Synthetic oligonucleotide
ggacaaggaggauggacgg

SEQ ID NO: 202 - RNA, 19 nt, Artificial Sequence
  Description of Artificial Sequence Synthetic oligonucleotide
gcuuccaagaauugcaucc

SEQ ID NO: 203 - RNA, 19 nt, Artificial Sequence
  Description of Artificial Sequence Synthetic oligonucleotide
acuucgggcuggcuaggga

SEQ ID NO: 204 - RNA, 19 nt, Artificial Sequence
  Description of Artificial Sequence Synthetic oligonucleotide
cuggcuagggacaucauga

SEQ ID NO: 205 - RNA, 19 nt, Artificial Sequence
  Description of Artificial Sequence Synthetic oligonucleotide
ggcuagggacaucaugaau

SEQ ID NO: 206 - RNA, 19 nt, Artificial Sequence
  Description of Artificial Sequence Synthetic oligonucleotide
uuugacugugucuacacgg

SEQ ID NO: 207 - RNA, 19 nt, Artificial Sequence
  Description of Artificial Sequence Synthetic oligonucleotide
ugacugugucuacacgguu

SEQ ID NO: 208 - RNA, 19 nt, Artificial Sequence
  Description of Artificial Sequence Synthetic oligonucleotide
acugugucuacacgguuca

SEQ ID NO: 209 - RNA, 19 nt, Artificial Sequence
  Description of Artificial Sequence Synthetic oligonucleotide
gugucuacacgguucagag

SEQ ID NO: 210 - RNA, 19 nt, Artificial Sequence
  Description of Artificial Sequence Synthetic oligonucleotide
cuacacgguucagagcgac

SEQ ID NO: 211 - RNA, 19 nt, Artificial Sequence
  Description of Artificial Sequence Synthetic oligonucleotide
cacgguucagagcgacguc

SEQ ID NO: 212 - RNA, 19 nt, Artificial Sequence
  Description of Artificial Sequence Synthetic oligonucleotide
gguucagagcgacgucugg

SEQ ID NO: 213 - RNA, 19 nt, Artificial Sequence
  Description of Artificial Sequence Synthetic oligonucleotide
uucagagcgacgucugguc

SEQ ID NO: 214 - RNA, 19 nt, Artificial Sequence
  Description of Artificial Sequence Synthetic oligonucleotide
cuugggcugaaucccuacc

SEQ ID NO: 215 - RNA, 19 nt, Artificial Sequence
  Description of Artificial Sequence Synthetic oligonucleotide
ugaacagcaaguucuauaa

SEQ ID NO: 216 - RNA, 19 nt, Artificial Sequence
  Description of Artificial Sequence Synthetic oligonucleotide
gcaaguucuauaaacuggu

SEQ ID NO: 217 - RNA, 19 nt, Artificial Sequence
  Description of Artificial Sequence Synthetic oligonucleotide
agaauauauacagcaucau

SEQ ID NO: 218 - RNA, 19 nt, Artificial Sequence
  Description of Artificial Sequence Synthetic oligonucleotide
agagagcgggacuauacca

SEQ ID NO: 219 - RNA, 19 nt, Artificial Sequence
  Description of Artificial Sequence Synthetic oligonucleotide
gagagcgggacuauaccaa

SEQ ID NO: 220 - RNA, 19 nt, Artificial Sequence
  Description of Artificial Sequence Synthetic oligonucleotide
agagcgggacuauaccaau

SEQ ID NO: 221 - RNA, 19 nt, Artificial Sequence
  Description of Artificial Sequence Synthetic oligonucleotide
gcgggacuauaccaaucug

SEQ ID NO: 222 - RNA, 19 nt, Artificial Sequence
  Description of Artificial Sequence Synthetic oligonucleotide
ggacuauaccaaucugccg

SEQ ID NO: 223 - RNA, 19 nt, Artificial Sequence
  Description of Artificial Sequence Synthetic oligonucleotide
ugcugcgagcaaggggaua

SEQ ID NO: 224 - RNA, 19 nt, Artificial Sequence
  Description of Artificial Sequence Synthetic oligonucleotide
gcgagcaaggggauaucgc

SEQ ID NO: 225 - RNA, 19 nt, Artificial Sequence
  Description of Artificial Sequence Synthetic oligonucleotide
cgagcaaggggauaucgcc

SEQ ID NO: 226 - RNA, 19 nt, Artificial Sequence
  Description of Artificial Sequence Synthetic oligonucleotide
gauaucgcccagcccuugc

SEQ ID NO: 227 - RNA, 19 nt, Artificial Sequence
  Description of Artificial Sequence Synthetic oligonucleotide
agcccaacaacuaucaguu

SEQ ID NO: 228 - RNA, 19 nt, Artificial Sequence
  Description of Artificial Sequence Synthetic oligonucleotide
gcccaacaacuaucaguuc

SEQ ID NO: 229 - RNA, 19 nt, Artificial Sequence
  Description of Artificial Sequence Synthetic oligonucleotide
cccaacaacuaucaguucu

SEQ ID NO: 230 - RNA, 19 nt, Artificial Sequence
  Description of Artificial Sequence Synthetic oligonucleotide
acaaacucugccuucgguc

SEQ ID NO: 231 - RNA, 19 nt, Artificial Sequence
  Description of Artificial Sequence Synthetic oligonucleotide
ucugccuucggucauuuca

SEQ ID NO: 232 - RNA, 19 nt, Artificial Sequence
  Description of Artificial Sequence Synthetic oligonucleotide
gccuucggucauuucacuc

SEQ ID NO: 233 - RNA, 19 nt, Artificial Sequence
  Description of Artificial Sequence Synthetic oligonucleotide
ucggucauuucacucaaca

SEQ ID NO: 234 - RNA, 19 nt, Artificial Sequence
  Description of Artificial Sequence Synthetic oligonucleotide
cucaugguguuggccucgu

SEQ ID NO: 235 - RNA, 19 nt, Artificial Sequence
  Description of Artificial Sequence Synthetic oligonucleotide
ucaugguguuggccucgug

SEQ ID NO: 236 - RNA, 19 nt, Artificial Sequence
  Description of Artificial Sequence Synthetic oligonucleotide
caugguguuggccucgugu

SEQ ID NO: 237 - RNA, 19 nt, Artificial Sequence
  Description of Artificial Sequence Synthetic oligonucleotide
gguguuggccucguguuug

SEQ ID NO: 238 - RNA, 19 nt, Artificial Sequence
  Description of Artificial Sequence Synthetic oligonucleotide
cguguuugcuaugccaacu

SEQ ID NO: 239 - RNA, 19 nt, Artificial Sequence
  Description of Artificial Sequence Synthetic oligonucleotide
agcuaaaaguugggggugu

SEQ ID NO: 240 - RNA, 19 nt, Artificial Sequence
  Description of Artificial Sequence Synthetic oligonucleotide
ccaagcugacucauccuaa

SEQ ID NO: 241 - RNA, 19 nt, Artificial Sequence
  Description of Artificial Sequence Synthetic oligonucleotide
agcugacucauccuaacua

SEQ ID NO: 242 - RNA, 19 nt, Artificial Sequence
  Description of Artificial Sequence Synthetic oligonucleotide
cugacucauccuaacuaac

SEQ ID NO: 243 - RNA, 19 nt, Artificial Sequence
  Description of Artificial Sequence Synthetic oligonucleotide
ugacucauccuaacuaaca

SEQ ID NO: 244 - RNA, 19 nt, Artificial Sequence
  Description of Artificial Sequence Synthetic oligonucleotide
gacucauccuaacuaacag

SEQ ID NO: 245 - RNA, 19 nt, Artificial Sequence
  Description of Artificial Sequence Synthetic oligonucleotide
uccuaacuaacagucacgc

SEQ ID NO: 246 - RNA, 19 nt, Artificial Sequence
  Description of Artificial Sequence Synthetic oligonucleotide
ccuaacuaacagucacgcc

SEQ ID NO: 247 - RNA, 19 nt, Artificial Sequence
  Description of Artificial Sequence Synthetic oligonucleotide
uccacauuaaacuaacagc

SEQ ID NO: 248 - RNA, 19 nt, Artificial Sequence
  Description of Artificial Sequence Synthetic oligonucleotide
ccacauuaaacuaacagca

SEQ ID NO: 249 - RNA, 19 nt, Artificial Sequence
  Description of Artificial Sequence Synthetic oligonucleotide
cucuaucacugggauuccc

SEQ ID NO: 250 - RNA, 19 nt, Artificial Sequence
  Description of Artificial Sequence Synthetic oligonucleotide
cauugcccacacaucgcaa

SEQ ID NO: 251 - RNA, 19 nt, Artificial Sequence
  Description of Artificial Sequence Synthetic oligonucleotide
gccauugcccacacaucgc

SEQ ID NO: 252 - RNA, 19 nt, Artificial Sequence
  Description of Artificial Sequence Synthetic oligonucleotide
guuuuggaagguagcguug

SEQ ID NO: 253 - RNA, 19 nt, Artificial Sequence
  Description of Artificial Sequence Synthetic oligonucleotide
guguuuuggaagguagcgu

SEQ ID NO: 254 - RNA, 19 nt, Artificial Sequence
  Description of Artificial Sequence Synthetic oligonucleotide
cguguuuuggaagguagcg

SEQ ID NO: 255 - RNA, 19 nt, Artificial Sequence
  Description of Artificial Sequence Synthetic oligonucleotide
cacguuccagggccgggca

SEQ ID NO: 256 - RNA, 19 nt, Artificial Sequence
  Description of Artificial Sequence Synthetic oligonucleotide
agcaccgggucugugagca

SEQ ID NO: 257 - RNA, 19 nt, Artificial Sequence
  Description of Artificial Sequence Synthetic oligonucleotide
acgcaccagcgagacgccu

SEQ ID NO: 258 - RNA, 19 nt, Artificial Sequence
  Description of Artificial Sequence Synthetic oligonucleotide
caucagggcacugcauuga

SEQ ID NO: 259 - RNA, 19 nt, Artificial Sequence
  Description of Artificial Sequence Synthetic oligonucleotide
cacuuucagccggaugcug

SEQ ID NO: 260 - RNA, 19 nt, Artificial Sequence
  Description of Artificial Sequence Synthetic oligonucleotide
cgaauccgcaccagcucug

SEQ ID NO: 261 - RNA, 19 nt, Artificial Sequence
  Description of Artificial Sequence Synthetic oligonucleotide
cucgaauccgcaccagcuc

SEQ ID NO: 262 - RNA, 19 nt, Artificial Sequence
  Description of Artificial Sequence Synthetic oligonucleotide
gccuccccucgaauccgca

SEQ ID NO: 263 - RNA, 19 nt, Artificial Sequence
  Description of Artificial Sequence Synthetic oligonucleotide
gagcacacgaucugggcag

SEQ ID NO: 264 - RNA, 19 nt, Artificial Sequence
  Description of Artificial Sequence Synthetic oligonucleotide
cugagcacacgaucugggc

SEQ ID NO: 265 - RNA, 19 nt, Artificial Sequence
  Description of Artificial Sequence Synthetic oligonucleotide
uggcugagcacacgaucug

SEQ ID NO: 266 - RNA, 19 nt, Artificial Sequence
  Description of Artificial Sequence Synthetic oligonucleotide
acuuuuugguaacgguuau

SEQ ID NO: 267 - RNA, 19 nt, Artificial Sequence
  Description of Artificial Sequence Synthetic oligonucleotide
ggacuuuuugguaacgguu

SEQ ID NO: 268 - RNA, 19 nt, Artificial Sequence
  Description of Artificial Sequence Synthetic oligonucleotide
aggacuuuuugguaacggu

SEQ ID NO: 269 - RNA, 19 nt, Artificial Sequence
  Description of Artificial Sequence Synthetic oligonucleotide
ggucaggacuuuuugguaa

SEQ ID NO: 270 - RNA, 19 nt, Artificial Sequence
  Description of Artificial Sequence Synthetic oligonucleotide
guagguccaguuaaaaccu

SEQ ID NO: 271 - RNA, 19 nt, Artificial Sequence
  Description of Artificial Sequence Synthetic oligonucleotide
gguagguccaguuaaaacc

SEQ ID NO: 272 - RNA, 19 nt, Artificial Sequence
  Description of Artificial Sequence Synthetic oligonucleotide
ccagguagguccaguuaaa

SEQ ID NO: 273 - RNA, 19 nt, Artificial Sequence
  Description of Artificial Sequence Synthetic oligonucleotide
cccagguagguccaguuaa

SEQ ID NO: 274 - RNA, 19 nt, Artificial Sequence
  Description of Artificial Sequence Synthetic oligonucleotide
aaagggucccagguagguc

SEQ ID NO: 275 - RNA, 19 nt, Artificial Sequence
  Description of Artificial Sequence Synthetic oligonucleotide
agggugagcucaaacguca

SEQ ID NO: 276 - RNA, 19 nt, Artificial Sequence
  Description of Artificial Sequence Synthetic oligonucleotide
aagggugagcucaaacguc

SEQ ID NO: 277 - RNA, 19 nt, Artificial Sequence
  Description of Artificial Sequence Synthetic oligonucleotide
uaucgaagggugagcucaa

SEQ ID NO: 278 - RNA, 19 nt, Artificial Sequence
  Description of Artificial Sequence Synthetic oligonucleotide
ggguaucgaagggugagcu

SEQ ID NO: 279 - RNA, 19 nt, Artificial Sequence
  Description of Artificial Sequence Synthetic oligonucleotide
accucugggggguaucgaa

SEQ ID NO: 280 - RNA, 19 nt, Artificial Sequence
  Description of Artificial Sequence Synthetic oligonucleotide
cuuaccucugggggguauc

SEQ ID NO: 281 - RNA, 19 nt, Artificial Sequence
  Description of Artificial Sequence Synthetic oligonucleotide
gguugugcucuaaggucuc

SEQ ID NO: 282 - RNA, 19 nt, Artificial Sequence
  Description of Artificial Sequence Synthetic oligonucleotide
uugguugugcucuaagguc

SEQ ID NO: 283 - RNA, 19 nt, Artificial Sequence
  Description of Artificial Sequence Synthetic oligonucleotide
ggagccacuccccacgcug

SEQ ID NO: 284 - RNA, 19 nt, Artificial Sequence
  Description of Artificial Sequence Synthetic oligonucleotide
aggaacucauccgggggau

SEQ ID NO: 285 - RNA, 19 nt, Artificial Sequence
  Description of Artificial Sequence Synthetic oligonucleotide
gaagaggaacucauccggg

SEQ ID NO: 286 - RNA, 19 nt, Artificial Sequence
  Description of Artificial Sequence Synthetic oligonucleotide
uggacaugcaggcgaccac

SEQ ID NO: 287 - RNA, 19 nt, Artificial Sequence
  Description of Artificial Sequence Synthetic oligonucleotide
uuauacuuguacaauagca

SEQ ID NO: 288 - RNA, 19 nt, Artificial Sequence
  Description of Artificial Sequence Synthetic oligonucleotide
ugaucuuccagcggaccug

SEQ ID NO: 289 - RNA, 19 nt, Artificial Sequence
  Description of Artificial Sequence Synthetic oligonucleotide
gaugaucuuccagcggacc

SEQ ID NO: 290 - RNA, 19 nt, Artificial Sequence
  Description of Artificial Sequence Synthetic oligonucleotide
ucgaugaucuuccagcgga

SEQ ID NO: 291 - RNA, 19 nt, Artificial Sequence
  Description of Artificial Sequence Synthetic oligonucleotide
cucgaugaucuuccagcgg

SEQ ID NO: 292 - RNA, 19 nt, Artificial Sequence
  Description of Artificial Sequence Synthetic oligonucleotide
ccucauagcucucgaugau

SEQ ID NO: 293 - RNA, 19 nt, Artificial Sequence
  Description of Artificial Sequence Synthetic oligonucleotide
gcccucauagcucucgaug

SEQ ID NO: 294 - RNA, 19 nt, Artificial Sequence
  Description of Artificial Sequence Synthetic oligonucleotide
uauaacuguugcccucaua

SEQ ID NO: 295 - RNA, 19 nt, Artificial Sequence
  Description of Artificial Sequence Synthetic oligonucleotide
aaaguauaacuguugcccu

SEQ ID NO: 296 - RNA, 19 nt, Artificial Sequence
  Description of Artificial Sequence Synthetic oligonucleotide
ugaaaguauaacuguugcc

SEQ ID NO: 297 - RNA, 19 nt, Artificial Sequence
  Description of Artificial Sequence Synthetic oligonucleotide
guuguaaggcagcugcgug

SEQ ID NO: 298 - RNA, 19 nt, Artificial Sequence
  Description of Artificial Sequence Synthetic oligonucleotide
gcagguuguuccgggggaa

SEQ ID NO: 299 - RNA, 19 nt, Artificial Sequence
  Description of Artificial Sequence Synthetic oligonucleotide
aaacugcagguuguuccgg

SEQ ID NO: 300 - RNA, 19 nt, Artificial Sequence
  Description of Artificial Sequence Synthetic oligonucleotide
cuccgagggucuuaccaaa

SEQ ID NO: 301 - RNA, 19 nt, Artificial Sequence
  Description of Artificial Sequence Synthetic oligonucleotide
agcuccgagggucuuacca

SEQ ID NO: 302 - RNA, 19 nt, Artificial Sequence
  Description of Artificial Sequence Synthetic oligonucleotide
gcaugggccguggacuuca

SEQ ID NO: 303 - RNA, 19 nt, Artificial Sequence
  Description of Artificial Sequence Synthetic oligonucleotide
agcaugggccguggacuuc

SEQ ID NO: 304 - RNA, 19 nt, Artificial Sequence
  Description of Artificial Sequence Synthetic oligonucleotide
ccgugaugaccaguacagg

SEQ ID NO: 305 - RNA, 19 nt, Artificial Sequence
  Description of Artificial Sequence Synthetic oligonucleotide
uccgugaugaccaguacag

SEQ ID NO: 306 - RNA, 19 nt, Artificial Sequence
  Description of Artificial Sequence Synthetic oligonucleotide
cuccgugaugaccaguaca

SEQ ID NO: 307 - RNA, 19 nt, Artificial Sequence
  Description of Artificial Sequence Synthetic oligonucleotide
uacuccgugaugaccagua

SEQ ID NO: 308 - RNA, 19 nt, Artificial Sequence
  Description of Artificial Sequence Synthetic oligonucleotide
acaguacuccgugaugacc

SEQ ID NO: 309 - RNA, 19 nt, Artificial Sequence
  Description of Artificial Sequence Synthetic oligonucleotide
agcaacaguacuccgugau

SEQ ID NO: 310 - RNA, 19 nt, Artificial Sequence
  Description of Artificial Sequence Synthetic oligonucleotide
uagcaacaguacuccguga

SEQ ID NO: 311 - RNA, 19 nt, Artificial Sequence
  Description of Artificial Sequence Synthetic oligonucleotide
cauagcaacaguacuccgu

SEQ ID NO: 312 - RNA, 19 nt, Artificial Sequence
  Description of Artificial Sequence Synthetic oligonucleotide
ucgccauagcaacaguacu

SEQ ID NO: 313 - RNA, 19 nt, Artificial Sequence
  Description of Artificial Sequence Synthetic oligonucleotide
gcaggucgccauagcaaca

SEQ ID NO: 314 - RNA, 19 nt, Artificial Sequence
  Description of Artificial Sequence Synthetic oligonucleotide
uugagcaggucgccauagc

SEQ ID NO: 315 - RNA, 19 nt, Artificial Sequence
  Description of Artificial Sequence Synthetic oligonucleotide
aaguugagcaggucgccau

SEQ ID NO: 316 - RNA, 19 nt, Artificial Sequence
  Description of Artificial Sequence Synthetic oligonucleotide
ccuucgcagaaaguugagc

SEQ ID NO: 317 - RNA, 19 nt, Artificial Sequence
  Description of Artificial Sequence Synthetic oligonucleotide
cuuccuucgcagaaaguug

SEQ ID NO: 318 - RNA, 19 nt, Artificial Sequence
  Description of Artificial Sequence Synthetic oligonucleotide
ucagccuuccuucgcagaa

SEQ ID NO: 319 - RNA, 19 nt, Artificial Sequence
  Description of Artificial Sequence Synthetic oligonucleotide
cauagguguccacacccug

SEQ ID NO: 320 - RNA, 19 nt, Artificial Sequence
  Description of Artificial Sequence Synthetic oligonucleotide
ccguccauccuccuugucc

SEQ ID NO: 321 - RNA, 19 nt, Artificial Sequence
  Description of Artificial Sequence Synthetic oligonucleotide
ggaugcaauucuuggaagc

SEQ ID NO: 322 - RNA, 19 nt, Artificial Sequence
  Description of Artificial Sequence Synthetic oligonucleotide
ucccuagccagcccgaagu

SEQ ID NO: 323 - RNA, 19 nt, Artificial Sequence
  Description of Artificial Sequence Synthetic oligonucleotide
ucaugaugucccuagccag

SEQ ID NO: 324 - RNA, 19 nt, Artificial Sequence
  Description of Artificial Sequence Synthetic oligonucleotide
auucaugaugucccuagcc

SEQ ID NO: 325 - RNA, 19 nt, Artificial Sequence
  Description of Artificial Sequence Synthetic oligonucleotide
ccguguagacacagucaaa

SEQ ID NO: 326 - RNA, 19 nt, Artificial Sequence
  Description of Artificial Sequence Synthetic oligonucleotide
aaccguguagacacaguca

SEQ ID NO: 327 - RNA, 19 nt, Artificial Sequence
  Description of Artificial Sequence Synthetic oligonucleotide
ugaaccguguagacacagu

SEQ ID NO: 328 - RNA, 19 nt, Artificial Sequence
  Description of Artificial Sequence Synthetic oligonucleotide
cucugaaccguguagacac

SEQ ID NO: 329 - RNA, 19 nt, Artificial Sequence
  Description of Artificial Sequence Synthetic oligonucleotide
gucgcucugaaccguguag

SEQ ID NO: 330 - RNA, 19 nt, Artificial Sequence
  Description of Artificial Sequence Synthetic oligonucleotide
gacgucgcucugaaccgug

SEQ ID NO: 331 - RNA, 19 nt, Artificial Sequence
  Description of Artificial Sequence Synthetic oligonucleotide
ccagacgucgcucugaacc

SEQ ID NO: 332 - RNA, 19 nt, Artificial Sequence
  Description of Artificial Sequence Synthetic oligonucleotide
gaccagacgucgcucugaa

SEQ ID NO: 333 - RNA, 19 nt, Artificial Sequence
  Description of Artificial Sequence Synthetic oligonucleotide
gguagggauucagcccaag

SEQ ID NO: 334 - RNA, 19 nt, Artificial Sequence
  Description of Artificial Sequence Synthetic oligonucleotide
uuauagaacuugcuguuca

SEQ ID NO: 335 - RNA, 19 nt, Artificial Sequence
  Description of Artificial Sequence Synthetic oligonucleotide
accaguuuauagaacuugc

SEQ ID NO: 336 - RNA, 19 nt, Artificial Sequence
  Description of Artificial Sequence Synthetic oligonucleotide
augaugcuguauauauucu

SEQ ID NO: 337 - RNA, 19 nt, Artificial Sequence
  Description of Artificial Sequence Synthetic oligonucleotide
ugguauagucccgcucucu

SEQ ID NO: 338 - RNA, 19 nt, Artificial Sequence
  Description of Artificial Sequence Synthetic oligonucleotide
uugguauagucccgcucuc

SEQ ID NO: 339 - RNA, 19 nt, Artificial Sequence
  Description of Artificial Sequence Synthetic oligonucleotide
auugguauagucccgcucu

SEQ ID NO: 340 - RNA, 19 nt, Artificial Sequence
  Description of Artificial Sequence Synthetic oligonucleotide
cagauugguauagucccgc

SEQ ID NO: 341 - RNA, 19 nt, Artificial Sequence
  Description of Artificial Sequence Synthetic oligonucleotide
cggcagauugguauagucc

SEQ ID NO: 342 - RNA, 19 nt, Artificial Sequence
  Description of Artificial Sequence Synthetic oligonucleotide
uauccccuugcucgcagca

SEQ ID NO: 343 - RNA, 19 nt, Artificial Sequence
  Description of Artificial Sequence Synthetic oligonucleotide
gcgauauccccuugcucgc

SEQ ID NO: 344 - RNA, 19 nt, Artificial Sequence
  Description of Artificial Sequence Synthetic oligonucleotide
ggcgauauccccuugcucg

SEQ ID NO: 345 - RNA, 19 nt, Artificial Sequence
  Description of Artificial Sequence Synthetic oligonucleotide
gcaagggcugggcgauauc

SEQ ID NO: 346 - RNA, 19 nt, Artificial Sequence
  Description of Artificial Sequence Synthetic oligonucleotide
aacugauaguuguugggcu

SEQ ID NO: 347 - RNA, 19 nt, Artificial Sequence
  Description of Artificial Sequence Synthetic oligonucleotide
gaacugauaguuguugggc

SEQ ID NO: 348 - RNA, 19 nt, Artificial Sequence
  Description of Artificial Sequence Synthetic oligonucleotide
agaacugauaguuguuggg

SEQ ID NO: 349 - RNA, 19 nt, Artificial Sequence
  Description of Artificial Sequence Synthetic oligonucleotide
gaccgaaggcagaguuugu

SEQ ID NO: 350 - RNA, 19 nt, Artificial Sequence
  Description of Artificial Sequence Synthetic oligonucleotide
ugaaaugaccgaaggcaga

SEQ ID NO: 351 - RNA, 19 nt, Artificial Sequence
  Description of Artificial Sequence Synthetic oligonucleotide
gagugaaaugaccgaaggc

SEQ ID NO: 352 - RNA, 19 nt, Artificial Sequence
  Description of Artificial Sequence Synthetic oligonucleotide
uguugagugaaaugaccga

SEQ ID NO: 353 - RNA, 19 nt, Artificial Sequence
  Description of Artificial Sequence Synthetic oligonucleotide
acgaggccaacaccaugag

SEQ ID NO: 354 - RNA, 19 nt, Artificial Sequence
  Description of Artificial Sequence Synthetic oligonucleotide
cacgaggccaacaccauga

SEQ ID NO: 355 - RNA, 19 nt, Artificial Sequence
  Description of Artificial Sequence Synthetic oligonucleotide
acacgaggccaacaccaug

SEQ ID NO: 356 - RNA, 19 nt, Artificial Sequence
  Description of Artificial Sequence Synthetic oligonucleotide
caaacacgaggccaacacc

SEQ ID NO: 357 - RNA, 19 nt, Artificial Sequence
  Description of Artificial Sequence Synthetic oligonucleotide
aguuggcauagcaaacacg

SEQ ID NO: 358 - RNA, 19 nt, Artificial Sequence
  Description of Artificial Sequence Synthetic oligonucleotide
acacccccaacuuuuagcu

SEQ ID NO: 359 - RNA, 19 nt, Artificial Sequence
  Description of Artificial Sequence Synthetic oligonucleotide
uuaggaugagucagcuugg

SEQ ID NO: 360 - RNA, 19 nt, Artificial Sequence
  Description of Artificial Sequence Synthetic oligonucleotide
uaguuaggaugagucagcu

SEQ ID NO: 361 - RNA, 19 nt, Artificial Sequence
  Description of Artificial Sequence Synthetic oligonucleotide
guuaguuaggaugagucag

SEQ ID NO: 362 - RNA, 19 nt, Artificial Sequence
  Description of Artificial Sequence Synthetic oligonucleotide
uguuaguuaggaugaguca

SEQ ID NO: 363 - RNA, 19 nt, Artificial Sequence
  Description of Artificial Sequence Synthetic oligonucleotide
cuguuaguuaggaugaguc

SEQ ID NO: 364 - RNA, 19 nt, Artificial Sequence
  Description of Artificial Sequence Synthetic oligonucleotide
gcgugacuguuaguuagga

SEQ ID NO: 365 - RNA, 19 nt, Artificial Sequence
  Description of Artificial Sequence Synthetic oligonucleotide
ggcgugacuguuaguuagg

SEQ ID NO: 366 - RNA, 19 nt, Artificial Sequence
  Description of Artificial Sequence Synthetic oligonucleotide
gcuguuaguuuaaugugga

SEQ ID NO: 367 - RNA, 19 nt, Artificial Sequence
  Description of Artificial Sequence Synthetic oligonucleotide
ugcuguuaguuuaaugugg

SEQ ID NO: 368 - DNA, 21 bp, Artificial Sequence
  Description of Artificial Sequence Synthetic oligonucleotide
  Description of Combined DNA/RNA Molecule Synthetic oligonucleotide
gggaaucccagugauagagtt

SEQ ID NO: 369 - DNA, 21 bp, Artificial Sequence
  Description of Artificial Sequence Synthetic oligonucleotide
  Description of Combined DNA/RNA Molecule Synthetic oligonucleotide
uugcgaugugugggcaaugtt

SEQ ID NO: 370 - DNA, 21 bp, Artificial Sequence
  Description of Artificial Sequence Synthetic oligonucleotide
  Description of Combined DNA/RNA Molecule Synthetic oligonucleotide
gcgaugugugggcaauggctt

SEQ ID NO: 371 - DNA, 21 bp, Artificial Sequence
  Description of Artificial Sequence Synthetic oligonucleotide
  Description of Combined DNA/RNA Molecule Synthetic oligonucleotide
caacgcuaccuuccaaaactt

SEQ ID NO: 372 - DNA, 21 bp, Artificial Sequence
  Description of Artificial Sequence Synthetic oligonucleotide
  Description of Combined DNA/RNA Molecule Synthetic oligonucleotide
acgcuaccuuccaaaacactt

SEQ ID NO: 373 - DNA, 21 bp, Artificial Sequence
  Description of Artificial Sequence Synthetic oligonucleotide
  Description of Combined DNA/RNA Molecule Synthetic oligonucleotide
cgcuaccuuccaaaacacgtt

SEQ ID NO: 374 - DNA, 21 bp, Artificial Sequence
  Description of Artificial Sequence Synthetic oligonucleotide
  Description of Combined DNA/RNA Molecule Synthetic oligonucleotide
ugcccggcccuggaacgugtt

SEQ ID NO: 375 - DNA, 21 bp, Artificial Sequence
  Description of Artificial Sequence Synthetic oligonucleotide
  Description of Combined DNA/RNA Molecule Synthetic oligonucleotide
ugcucacagacccggugcutt

SEQ ID NO: 376 - DNA, 21 bp, Artificial Sequence
  Description of Artificial Sequence Synthetic oligonucleotide
  Description of Combined DNA/RNA Molecule Synthetic oligonucleotide
aggcgucucgcuggugcgutt

SEQ ID NO: 377 - DNA, 21 bp, Artificial Sequence
  Description of Artificial Sequence Synthetic oligonucleotide
  Description of Combined DNA/RNA Molecule Synthetic oligonucleotide
ucaaugcagugcccugaugtt

SEQ ID NO: 378 - DNA, 21 bp, Artificial Sequence
  Description of Artificial Sequence Synthetic oligonucleotide
  Description of Combined DNA/RNA Molecule Synthetic oligonucleotide
cagcauccggcugaaagugtt

SEQ ID NO: 379 - DNA, 21 bp, Artificial Sequence
  Description of Artificial Sequence Synthetic oligonucleotide
  Description of Combined DNA/RNA Molecule Synthetic oligonucleotide
cagagcuggugcggauucgtt

SEQ ID NO: 380 - DNA, 21 bp, Artificial Sequence
  Description of Artificial Sequence Synthetic oligonucleotide
  Description of Combined DNA/RNA Molecule Synthetic oligonucleotide
gagcuggugcggauucgagtt

SEQ ID NO: 381 - DNA, 21 bp, Artificial Sequence
  Description of Artificial Sequence Synthetic oligonucleotide
  Description of Combined DNA/RNA Molecule Synthetic oligonucleotide
ugcggauucgaggggaggctt

SEQ ID NO: 382 - DNA, 21 bp, Artificial Sequence
  Description of Artificial Sequence Synthetic oligonucleotide
  Description of Combined DNA/RNA Molecule Synthetic oligonucleotide
cugcccagaucgugugcuctt

SEQ ID NO: 383 - DNA, 21 bp, Artificial Sequence
  Description of Artificial Sequence Synthetic oligonucleotide
  Description of Combined DNA/RNA Molecule Synthetic oligonucleotide
gcccagaucgugugcucagtt

SEQ ID NO: 384 - DNA, 21 bp, Artificial Sequence
  Description of Artificial Sequence Synthetic oligonucleotide
  Description of Combined DNA/RNA Molecule Synthetic oligonucleotide
cagaucgugugcucagccatt

SEQ ID NO: 385 - DNA, 21 bp, Artificial Sequence
  Description of Artificial Sequence Synthetic oligonucleotide
  Description of Combined DNA/RNA Molecule Synthetic oligonucleotide
auaaccguuaccaaaaagutt

SEQ ID NO: 386 - DNA, 21 bp, Artificial Sequence
  Description of Artificial Sequence Synthetic oligonucleotide
  Description of Combined DNA/RNA Molecule Synthetic oligonucleotide
aaccguuaccaaaaagucctt

SEQ ID NO: 387 - DNA, 21 bp, Artificial Sequence
  Description of Artificial Sequence Synthetic oligonucleotide
  Description of Combined DNA/RNA Molecule Synthetic oligonucleotide
accguuaccaaaaaguccutt

SEQ ID NO: 388 - DNA, 21 bp, Artificial Sequence
  Description of Artificial Sequence Synthetic oligonucleotide
  Description of Combined DNA/RNA Molecule Synthetic oligonucleotide
uuaccaaaaaguccugacctt

SEQ ID NO: 389 - DNA, 21 bp, Artificial Sequence
  Description of Artificial Sequence Synthetic oligonucleotide
  Description of Combined DNA/RNA Molecule Synthetic oligonucleotide
agguuuuaacuggaccuactt

SEQ ID NO: 390 - DNA, 21 bp, Artificial Sequence
  Description of Artificial Sequence Synthetic oligonucleotide
  Description of Combined DNA/RNA Molecule Synthetic oligonucleotide
gguuuuaacuggaccuacctt

SEQ ID NO: 391 - DNA, 21 bp, Artificial Sequence
  Description of Artificial Sequence Synthetic oligonucleotide
  Description of Combined DNA/RNA Molecule Synthetic oligonucleotide
uuuaacuggaccuaccuggtt

SEQ ID NO: 392 - DNA, 21 bp, Artificial Sequence
  Description of Artificial Sequence Synthetic oligonucleotide
  Description of Combined DNA/RNA Molecule Synthetic oligonucleotide
uuaacuggaccuaccugggtt

SEQ ID NO: 393 - DNA, 21 bp, Artificial Sequence
  Description of Artificial Sequence Synthetic oligonucleotide
  Description of Combined DNA/RNA Molecule Synthetic oligonucleotide
gaccuaccugggacccuuutt

SEQ ID NO: 394 - DNA, 21 bp, Artificial Sequence
  Description of Artificial Sequence Synthetic oligonucleotide
  Description of Combined DNA/RNA Molecule Synthetic oligonucleotide
ugacguuugagcucacccutt

SEQ ID NO: 395 - DNA, 21 bp, Artificial Sequence
  Description of Artificial Sequence Synthetic oligonucleotide
  Description of Combined DNA/RNA Molecule Synthetic oligonucleotide
gacguuugagcucacccuutt

SEQ ID NO: 396 - DNA, 21 bp, Artificial Sequence
  Description of Artificial Sequence Synthetic oligonucleotide
  Description of Combined DNA/RNA Molecule Synthetic oligonucleotide
uugagcucacccuucgauatt

SEQ ID NO: 397 - DNA, 21 bp, Artificial Sequence
  Description of Artificial Sequence Synthetic oligonucleotide
  Description of Combined DNA/RNA Molecule Synthetic oligonucleotide
agcucacccuucgauaccctt

SEQ ID NO: 398 - DNA, 21 bp, Artificial Sequence
  Description of Artificial Sequence Synthetic oligonucleotide
  Description of Combined DNA/RNA Molecule Synthetic oligonucleotide
uucgauaccccccagaggutt

SEQ ID NO: 399 - DNA, 21 bp, Artificial Sequence
  Description of Artificial Sequence Synthetic oligonucleotide
  Description of Combined DNA/RNA Molecule Synthetic oligonucleotide
gauaccccccagagguaagtt

SEQ ID NO: 400 - DNA, 21 bp, Artificial Sequence
  Description of Artificial Sequence Synthetic oligonucleotide
  Description of Combined DNA/RNA Molecule Synthetic oligonucleotide
gagaccuuagagcacaacctt

SEQ ID NO: 401 - DNA, 21 bp, Artificial Sequence
  Description of Artificial Sequence Synthetic oligonucleotide
  Description of Combined DNA/RNA Molecule Synthetic oligonucleotide
gaccuuagagcacaaccaatt

SEQ ID NO: 402 - DNA, 21 bp, Artificial Sequence
  Description of Artificial Sequence Synthetic oligonucleotide
  Description of Combined DNA/RNA Molecule Synthetic oligonucleotide
cagcguggggaguggcucctt

SEQ ID NO: 403 - DNA, 21 bp, Artificial Sequence
  Description of Artificial Sequence Synthetic oligonucleotide
  Description of Combined DNA/RNA Molecule Synthetic oligonucleotide
aucccccggaugaguuccutt

SEQ ID NO: 404 - DNA, 21 bp, Artificial Sequence
  Description of Artificial Sequence Synthetic oligonucleotide
  Description of Combined DNA/RNA Molecule Synthetic oligonucleotide
cccggaugaguuccucuuctt

SEQ ID NO: 405 - DNA, 21 bp, Artificial Sequence
  Description of Artificial Sequence Synthetic oligonucleotide
  Description of Combined DNA/RNA Molecule Synthetic oligonucleotide
guggucgccugcauguccatt

SEQ ID NO: 406 - DNA, 21 bp, Artificial Sequence
  Description of Artificial Sequence Synthetic oligonucleotide
  Description of Combined DNA/RNA Molecule Synthetic oligonucleotide
ugcuauuguacaaguauaatt

SEQ ID NO: 407 - DNA, 21 bp, Artificial Sequence
  Description of Artificial Sequence Synthetic oligonucleotide
  Description of Combined DNA/RNA Molecule Synthetic oligonucleotide
cagguccgcuggaagaucatt

SEQ ID NO: 408 - DNA, 21 bp, Artificial Sequence
  Description of Artificial Sequence Synthetic oligonucleotide
  Description of Combined DNA/RNA Molecule Synthetic oligonucleotide
gguccgcuggaagaucauctt

SEQ ID NO: 409 - DNA, 21 bp, Artificial Sequence
  Description of Artificial Sequence Synthetic oligonucleotide
  Description of Combined DNA/RNA Molecule Synthetic oligonucleotide
uccgcuggaagaucaucgatt

SEQ ID NO: 410 - DNA, 21 bp, Artificial Sequence
  Description of Artificial Sequence Synthetic oligonucleotide
  Description of Combined DNA/RNA Molecule Synthetic oligonucleotide
ccgcuggaagaucaucgagtt

SEQ ID NO: 411 - DNA, 21 bp, Artificial Sequence
  Description of Artificial Sequence Synthetic oligonucleotide
  Description of Combined DNA/RNA Molecule Synthetic oligonucleotide
aucaucgagagcuaugaggtt

SEQ ID NO: 412 - DNA, 21 bp, Artificial Sequence
  Description of Artificial Sequence Synthetic oligonucleotide
  Description of Combined DNA/RNA Molecule Synthetic oligonucleotide
caucgagagcuaugagggctt

SEQ ID NO: 413 - DNA, 21 bp, Artificial Sequence
  Description of Artificial Sequence Synthetic oligonucleotide
  Description of Combined DNA/RNA Molecule Synthetic oligonucleotide
uaugagggcaacaguuauatt

SEQ ID NO: 414 - DNA, 21 bp, Artificial Sequence
  Description of Artificial Sequence Synthetic oligonucleotide
  Description of Combined DNA/RNA Molecule Synthetic oligonucleotide
agggcaacaguuauacuuutt

SEQ ID NO: 415 - DNA, 21 bp, Artificial Sequence
  Description of Artificial Sequence Synthetic oligonucleotide
  Description of Combined DNA/RNA Molecule Synthetic oligonucleotide
ggcaacaguuauacuuucatt

SEQ ID NO: 416 - DNA, 21 bp, Artificial Sequence
  Description of Artificial Sequence Synthetic oligonucleotide
  Description of Combined DNA/RNA Molecule Synthetic oligonucleotide
cacgcagcugccuuacaactt

SEQ ID NO: 417 - DNA, 21 bp, Artificial Sequence
  Description of Artificial Sequence Synthetic oligonucleotide
  Description of Combined DNA/RNA Molecule Synthetic oligonucleotide
uucccccggaacaaccugctt

SEQ ID NO: 418 - DNA, 21 bp, Artificial Sequence
  Description of Artificial Sequence Synthetic oligonucleotide
  Description of Combined DNA/RNA Molecule Synthetic oligonucleotide
ccggaacaaccugcaguuutt

SEQ ID NO: 419 - DNA, 21 bp, Artificial Sequence
  Description of Artificial Sequence Synthetic oligonucleotide
  Description of Combined DNA/RNA Molecule Synthetic oligonucleotide
uuugguaagacccucggagtt

SEQ ID NO: 420 - DNA, 21 bp, Artificial Sequence
  Description of Artificial Sequence Synthetic oligonucleotide
  Description of Combined DNA/RNA Molecule Synthetic oligonucleotide
ugguaagacccucggagcutt

SEQ ID NO: 421 - DNA, 21 bp, Artificial Sequence
  Description of Artificial Sequence Synthetic oligonucleotide
  Description of Combined DNA/RNA Molecule Synthetic oligonucleotide
ugaaguccacggcccaugctt

SEQ ID NO: 422 - DNA, 21 bp, Artificial Sequence
  Description of Artificial Sequence Synthetic oligonucleotide
  Description of Combined DNA/RNA Molecule Synthetic oligonucleotide
gaaguccacggcccaugcutt

SEQ ID NO: 423 - DNA, 21 bp, Artificial Sequence
  Description of Artificial Sequence Synthetic oligonucleotide
  Description of Combined DNA/RNA Molecule Synthetic oligonucleotide
ccuguacuggucaucacggtt

SEQ ID NO: 424 - DNA, 21 bp, Artificial Sequence
  Description of Artificial Sequence Synthetic oligonucleotide
  Description of Combined DNA/RNA Molecule Synthetic oligonucleotide
cuguacuggucaucacggatt

SEQ ID NO: 425 - DNA, 21 bp, Artificial Sequence
  Description of Artificial Sequence Synthetic oligonucleotide
  Description of Combined DNA/RNA Molecule Synthetic oligonucleotide
uguacuggucaucacggagtt

SEQ ID NO: 426 - DNA, 21 bp, Artificial Sequence
  Description of Artificial Sequence Synthetic oligonucleotide
  Description of Combined DNA/RNA Molecule Synthetic oligonucleotide
uacuggucaucacggaguatt

SEQ ID NO: 427 - DNA, 21 bp, Artificial Sequence
  Description of Artificial Sequence Synthetic oligonucleotide
  Description of Combined DNA/RNA Molecule Synthetic oligonucleotide
ggucaucacggaguacugutt

SEQ ID NO: 428 - DNA, 21 bp, Artificial Sequence
  Description of Artificial Sequence Synthetic oligonucleotide
  Description of Combined DNA/RNA Molecule Synthetic oligonucleotide
aucacggaguacuguugcutt

SEQ ID NO: 429 - DNA, 21 bp, Artificial Sequence
  Description of Artificial Sequence Synthetic oligonucleotide
  Description of Combined DNA/RNA Molecule Synthetic oligonucleotide
ucacggaguacuguugcuatt

SEQ ID NO: 430 - DNA, 21 bp, Artificial Sequence
  Description of Artificial Sequence Synthetic oligonucleotide
  Description of Combined DNA/RNA Molecule Synthetic oligonucleotide
acggaguacuguugcuaugtt

SEQ ID NO: 431 - DNA, 21 bp, Artificial Sequence
  Description of Artificial Sequence Synthetic oligonucleotide
  Description of Combined DNA/RNA Molecule Synthetic oligonucleotide
aguacuguugcuauggcgatt

SEQ ID NO: 432 - DNA, 21 bp, Artificial Sequence
  Description of Artificial Sequence Synthetic oligonucleotide
  Description of Combined DNA/RNA Molecule Synthetic oligonucleotide
uguugcuauggcgaccugctt

SEQ ID NO: 433 - DNA, 21 bp, Artificial Sequence
  Description of Artificial Sequence Synthetic oligonucleotide
  Description of Combined DNA/RNA Molecule Synthetic oligonucleotide
gcuauggcgaccugcucaatt

SEQ ID NO: 434 - DNA, 21 bp, Artificial Sequence
  Description of Artificial Sequence Synthetic oligonucleotide
  Description of Combined DNA/RNA Molecule Synthetic oligonucleotide
auggcgaccugcucaacuutt

SEQ ID NO: 435 - DNA, 21 bp, Artificial Sequence
  Description of Artificial Sequence Synthetic oligonucleotide
  Description of Combined DNA/RNA Molecule Synthetic oligonucleotide
gcucaacuuucugcgaaggtt

SEQ ID NO: 436 - DNA, 21 bp, Artificial Sequence
  Description of Artificial Sequence Synthetic oligonucleotide
  Description of Combined DNA/RNA Molecule Synthetic oligonucleotide
caacuuucugcgaaggaagtt

SEQ ID NO: 437 - DNA, 21 bp, Artificial Sequence
  Description of Artificial Sequence Synthetic oligonucleotide
  Description of Combined DNA/RNA Molecule Synthetic oligonucleotide
uucugcgaaggaaggcugatt

SEQ ID NO: 438 - DNA, 21 bp, Artificial Sequence
  Description of Artificial Sequence Synthetic oligonucleotide
  Description of Combined DNA/RNA Molecule Synthetic oligonucleotide
caggguguggacaccuaugtt

SEQ ID NO: 439 - DNA, 21 bp, Artificial Sequence
  Description of Artificial Sequence Synthetic oligonucleotide
  Description of Combined DNA/RNA Molecule Synthetic oligonucleotide
ggacaaggaggauggacggtt

SEQ ID NO: 440 - DNA, 21 bp, Artificial Sequence
  Description of Artificial Sequence Synthetic oligonucleotide
  Description of Combined DNA/RNA Molecule Synthetic oligonucleotide
gcuuccaagaauugcaucctt

SEQ ID NO: 441 - DNA, 21 bp, Artificial Sequence
  Description of Artificial Sequence Synthetic oligonucleotide
  Description of Combined DNA/RNA Molecule Synthetic oligonucleotide
acuucgggcuggcuagggatt

SEQ ID NO: 442 - DNA, 21 bp, Artificial Sequence
  Description of Artificial Sequence Synthetic oligonucleotide
  Description of Combined DNA/RNA Molecule Synthetic oligonucleotide
cuggcuagggacaucaugatt

SEQ ID NO: 443 - DNA, 21 bp, Artificial Sequence
  Description of Artificial Sequence Synthetic oligonucleotide
  Description of Combined DNA/RNA Molecule Synthetic oligonucleotide
ggcuagggacaucaugaautt

SEQ ID NO: 444 - DNA, 21 bp, Artificial Sequence
  Description of Artificial Sequence Synthetic oligonucleotide
  Description of Combined DNA/RNA Molecule Synthetic oligonucleotide
uuugacugugucuacacggtt

SEQ ID NO: 445 - DNA, 21 bp, Artificial Sequence
  Description of Artificial Sequence Synthetic oligonucleotide
  Description of Combined DNA/RNA Molecule Synthetic oligonucleotide
ugacugugucuacacgguutt

SEQ ID NO: 446 - DNA, 21 bp, Artificial Sequence
  Description of Artificial Sequence Synthetic oligonucleotide
  Description of Combined DNA/RNA Molecule Synthetic oligonucleotide
acugugucuacacgguucatt

SEQ ID NO: 447 - DNA, 21 bp, Artificial Sequence
  Description of Artificial Sequence Synthetic oligonucleotide
  Description of Combined DNA/RNA Molecule Synthetic oligonucleotide
gugucuacacgguucagagtt

SEQ ID NO: 448 - DNA, 21 bp, Artificial Sequence
  Description of Artificial Sequence Synthetic oligonucleotide
  Description of Combined DNA/RNA Molecule Synthetic oligonucleotide
cuacacgguucagagcgactt

SEQ ID NO: 449 - DNA, 21 bp, Artificial Sequence
  Description of Artificial Sequence Synthetic oligonucleotide
  Description of Combined DNA/RNA Molecule Synthetic oligonucleotide
cacgguucagagcgacguctt

SEQ ID NO: 450 - DNA, 21 bp, Artificial Sequence
  Description of Artificial Sequence Synthetic oligonucleotide
  Description of Combined DNA/RNA Molecule Synthetic oligonucleotide
gguucagagcgacgucuggtt

SEQ ID NO: 451 - DNA, 21 bp, Artificial Sequence
  Description of Artificial Sequence Synthetic oligonucleotide
  Description of Combined DNA/RNA Molecule Synthetic oligonucleotide
uucagagcgacgucugguctt

SEQ ID NO: 452 - DNA, 21 bp, Artificial Sequence
  Description of Artificial Sequence Synthetic oligonucleotide
  Description of Combined DNA/RNA Molecule Synthetic oligonucleotide
cuugggcugaaucccuacctt

SEQ ID NO: 453 - DNA, 21 bp, Artificial Sequence
  Description of Artificial Sequence Synthetic oligonucleotide
  Description of Combined DNA/RNA Molecule Synthetic oligonucleotide
ugaacagcaaguucuauaatt

SEQ ID NO: 454 - DNA, 21 bp, Artificial Sequence
  Description of Artificial Sequence Synthetic oligonucleotide
  Description of Combined DNA/RNA Molecule Synthetic oligonucleotide
gcaaguucuauaaacuggutt

SEQ ID NO: 455 - DNA, 21 bp, Artificial Sequence
  Description of Artificial Sequence Synthetic oligonucleotide
  Description of Combined DNA/RNA Molecule Synthetic oligonucleotide
agaauauauacagcaucautt

SEQ ID NO: 456 - DNA, 21 bp, Artificial Sequence
  Description of Artificial Sequence Synthetic oligonucleotide
  Description of Combined DNA/RNA Molecule Synthetic oligonucleotide
agagagcgggacuauaccatt

SEQ ID NO: 457 - DNA, 21 bp, Artificial Sequence
  Description of Artificial Sequence Synthetic oligonucleotide
  Description of Combined DNA/RNA Molecule Synthetic oligonucleotide
gagagcgggacuauaccaatt

SEQ ID NO: 458 - DNA, 21 bp, Artificial Sequence
  Description of Artificial Sequence Synthetic oligonucleotide
  Description of Combined DNA/RNA Molecule Synthetic oligonucleotide
agagcgggacuauaccaautt

SEQ ID NO: 459 - DNA, 21 bp, Artificial Sequence
  Description of Artificial Sequence Synthetic oligonucleotide
  Description of Combined DNA/RNA Molecule Synthetic oligonucleotide
gcgggacuauaccaaucugtt

SEQ ID NO: 460 - DNA, 21 bp, Artificial Sequence
  Description of Artificial Sequence Synthetic oligonucleotide
  Description of Combined DNA/RNA Molecule Synthetic oligonucleotide
ggacuauaccaaucugccgtt

SEQ ID NO: 461 - DNA, 21 bp, Artificial Sequence
  Description of Artificial Sequence Synthetic oligonucleotide
  Description of Combined DNA/RNA Molecule Synthetic oligonucleotide
ugcugcgagcaaggggauatt

SEQ ID NO: 462 - DNA, 21 bp, Artificial Sequence
  Description of Artificial Sequence Synthetic oligonucleotide
  Description of Combined DNA/RNA Molecule Synthetic oligonucleotide
gcgagcaaggggauaucgctt

SEQ ID NO: 463 - DNA, 21 bp, Artificial Sequence
  Description of Artificial Sequence Synthetic oligonucleotide
  Description of Combined DNA/RNA Molecule Synthetic oligonucleotide
cgagcaaggggauaucgcctt

SEQ ID NO: 464 - DNA, 21 bp, Artificial Sequence
  Description of Artificial Sequence Synthetic oligonucleotide
  Description of Combined DNA/RNA Molecule Synthetic oligonucleotide
gauaucgcccagcccuugctt

SEQ ID NO: 465 - DNA, 21 bp, Artificial Sequence
  Description of Artificial Sequence Synthetic oligonucleotide
  Description of Combined DNA/RNA Molecule Synthetic oligonucleotide
agcccaacaacuaucaguutt

SEQ ID NO: 466 - DNA, 21 bp, Artificial Sequence
  Description of Artificial Sequence Synthetic oligonucleotide
  Description of Combined DNA/RNA Molecule Synthetic oligonucleotide
gcccaacaacuaucaguuctt

SEQ ID NO: 467 - DNA, 21 bp, Artificial Sequence
  Description of Artificial Sequence Synthetic oligonucleotide
  Description of Combined DNA/RNA Molecule Synthetic oligonucleotide
cccaacaacuaucaguucutt

SEQ ID NO: 468 - DNA, 21 bp, Artificial Sequence
  Description of Artificial Sequence Synthetic oligonucleotide
  Description of Combined DNA/RNA Molecule Synthetic oligonucleotide
acaaacucugccuucgguctt

SEQ ID NO: 469 - DNA, 21 bp, Artificial Sequence
  Description of Artificial Sequence Synthetic oligonucleotide
  Description of Combined DNA/RNA Molecule Synthetic oligonucleotide
ucugccuucggucauuucatt

SEQ ID NO: 470 - DNA, 21 bp, Artificial Sequence
  Description of Artificial Sequence Synthetic oligonucleotide
  Description of Combined DNA/RNA Molecule Synthetic oligonucleotide
gccuucggucauuucacuctt

SEQ ID NO: 471 - DNA, 21 bp, Artificial Sequence
  Description of Artificial Sequence Synthetic oligonucleotide
  Description of Combined DNA/RNA Molecule Synthetic oligonucleotide
ucggucauuucacucaacatt

SEQ ID NO: 472 - DNA, 21 bp, Artificial Sequence
  Description of Artificial Sequence Synthetic oligonucleotide
  Description of Combined DNA/RNA Molecule Synthetic oligonucleotide
cucaugguguuggccucgutt

SEQ ID NO: 473 - DNA, 21 bp, Artificial Sequence
  Description of Artificial Sequence Synthetic oligonucleotide
  Description of Combined DNA/RNA Molecule Synthetic oligonucleotide
ucaugguguuggccucgugtt

SEQ ID NO: 474 - DNA, 21 bp, Artificial Sequence
  Description of Artificial Sequence Synthetic oligonucleotide
  Description of Combined DNA/RNA Molecule Synthetic oligonucleotide
caugguguuggccucgugutt

SEQ ID NO: 475 - DNA, 21 bp, Artificial Sequence
  Description of Artificial Sequence Synthetic oligonucleotide
  Description of Combined DNA/RNA Molecule Synthetic oligonucleotide
gguguuggccucguguuugtt

SEQ ID NO: 476 - DNA, 21 bp, Artificial Sequence
  Description of Artificial Sequence Synthetic oligonucleotide
  Description of Combined DNA/RNA Molecule Synthetic oligonucleotide
cguguuugcuaugccaacutt

SEQ ID NO: 477 - DNA, 21 bp, Artificial Sequence
  Description of Artificial Sequence Synthetic oligonucleotide
  Description of Combined DNA/RNA Molecule Synthetic oligonucleotide
agcuaaaaguugggggugutt

SEQ ID NO: 478 - DNA, 21 bp, Artificial Sequence
  Description of Artificial Sequence Synthetic oligonucleotide
  Description of Combined DNA/RNA Molecule Synthetic oligonucleotide
ccaagcugacucauccuaatt

SEQ ID NO: 479 - DNA, 21 bp, Artificial Sequence
  Description of Artificial Sequence Synthetic oligonucleotide
  Description of Combined DNA/RNA Molecule Synthetic oligonucleotide
agcugacucauccuaacuatt

SEQ ID NO: 480 - DNA, 21 bp, Artificial Sequence
  Description of Artificial Sequence Synthetic oligonucleotide
  Description of Combined DNA/RNA Molecule Synthetic oligonucleotide
cugacucauccuaacuaactt

SEQ ID NO: 481 - DNA, 21 bp, Artificial Sequence
  Description of Artificial Sequence Synthetic oligonucleotide
  Description of Combined DNA/RNA Molecule Synthetic oligonucleotide
ugacucauccuaacuaacatt

SEQ ID NO: 482 - DNA, 21 bp, Artificial Sequence
  Description of Artificial Sequence Synthetic oligonucleotide
  Description of Combined DNA/RNA Molecule Synthetic oligonucleotide
gacucauccuaacuaacagtt

SEQ ID NO: 483 - DNA, 21 bp, Artificial Sequence
  Description of Artificial Sequence Synthetic oligonucleotide
  Description of Combined DNA/RNA Molecule Synthetic oligonucleotide
uccuaacuaacagucacgctt

SEQ ID NO: 484 - DNA, 21 bp, Artificial Sequence
  Description of Artificial Sequence Synthetic oligonucleotide
  Description of Combined DNA/RNA Molecule Synthetic oligonucleotide
ccuaacuaacagucacgcctt

SEQ ID NO: 485 - DNA, 21 bp, Artificial Sequence
  Description of Artificial Sequence Synthetic oligonucleotide
  Description of Combined DNA/RNA Molecule Synthetic oligonucleotide
uccacauuaaacuaacagctt

SEQ ID NO: 486 - DNA, 21 bp, Artificial Sequence
  Description of Artificial Sequence Synthetic oligonucleotide
  Description of Combined DNA/RNA Molecule Synthetic oligonucleotide
ccacauuaaacuaacagcatt

SEQ ID NO: 487 - DNA, 21 bp, Artificial Sequence
  Description of Artificial Sequence Synthetic oligonucleotide
  Description of Combined DNA/RNA Molecule Synthetic oligonucleotide
cucuaucacugggauuccctt

SEQ ID NO: 488 - DNA, 21 bp, Artificial Sequence
  Description of Artificial Sequence Synthetic oligonucleotide
  Description of Combined DNA/RNA Molecule Synthetic oligonucleotide
cauugcccacacaucgcaatt

SEQ ID NO: 489 - DNA, 21 bp, Artificial Sequence
  Description of Artificial Sequence Synthetic oligonucleotide
  Description of Combined DNA/RNA Molecule Synthetic oligonucleotide
gccauugcccacacaucgctt

SEQ ID NO: 490 - DNA, 21 bp, Artificial Sequence
  Description of Artificial Sequence Synthetic oligonucleotide
  Description of Combined DNA/RNA Molecule Synthetic oligonucleotide
guuuuggaagguagcguugtt

SEQ ID NO: 491 - DNA, 21 bp, Artificial Sequence
  Description of Artificial Sequence Synthetic oligonucleotide
  Description of Combined DNA/RNA Molecule Synthetic oligonucleotide
guguuuuggaagguagcgutt

SEQ ID NO: 492 - DNA, 21 bp, Artificial Sequence
  Description of Artificial Sequence Synthetic oligonucleotide
  Description of Combined DNA/RNA Molecule Synthetic oligonucleotide
cguguuuuggaagguagcgtt

SEQ ID NO: 493 - DNA, 21 bp, Artificial Sequence
  Description of Artificial Sequence Synthetic oligonucleotide
  Description of Combined DNA/RNA Molecule Synthetic oligonucleotide
cacguuccagggccgggcatt

SEQ ID NO: 494 - DNA, 21 bp, Artificial Sequence
  Description of Artificial Sequence Synthetic oligonucleotide
  Description of Combined DNA/RNA Molecule Synthetic oligonucleotide
agcaccgggucugugagcatt

SEQ ID NO: 495 - DNA, 21 bp, Artificial Sequence
  Description of Artificial Sequence Synthetic oligonucleotide
  Description of Combined DNA/RNA Molecule Synthetic oligonucleotide
acgcaccagcgagacgccutt

SEQ ID NO: 496 - DNA, 21 bp, Artificial Sequence
  Description of Artificial Sequence Synthetic oligonucleotide
  Description of Combined DNA/RNA Molecule Synthetic oligonucleotide
caucagggcacugcauugatt

SEQ ID NO: 497 - DNA, 21 bp, Artificial Sequence
  Description of Artificial Sequence Synthetic oligonucleotide
  Description of Combined DNA/RNA Molecule Synthetic oligonucleotide
cacuuucagccggaugcugtt

SEQ ID NO: 498 - DNA, 21 bp, Artificial Sequence
  Description of Artificial Sequence Synthetic oligonucleotide
  Description of Combined DNA/RNA Molecule Synthetic oligonucleotide
cgaauccgcaccagcucugtt

SEQ ID NO: 499 - DNA, 21 bp, Artificial Sequence
  Description of Artificial Sequence Synthetic oligonucleotide
  Description of Combined DNA/RNA Molecule Synthetic oligonucleotide
cucgaauccgcaccagcuctt

SEQ ID NO: 500 - DNA, 21 bp, Artificial Sequence
  Description of Artificial Sequence Synthetic oligonucleotide
  Description of Combined DNA/RNA Molecule Synthetic oligonucleotide
gccuccccucgaauccgcatt

SEQ ID NO: 501 - DNA, 21 bp, Artificial Sequence
  Description of Artificial Sequence Synthetic oligonucleotide
  Description of Combined DNA/RNA Molecule Synthetic oligonucleotide
gagcacacgaucugggcagtt

SEQ ID NO: 502 - DNA, 21 bp, Artificial Sequence
  Description of Artificial Sequence Synthetic oligonucleotide
  Description of Combined DNA/RNA Molecule Synthetic oligonucleotide
cugagcacacgaucugggctt

SEQ ID NO: 503 - DNA, 21 bp, Artificial Sequence
  Description of Artificial Sequence Synthetic oligonucleotide
  Description of Combined DNA/RNA Molecule Synthetic oligonucleotide
uggcugagcacacgaucugtt

SEQ ID NO: 504 - DNA, 21 bp, Artificial Sequence
  Description of Artificial Sequence Synthetic oligonucleotide
  Description of Combined DNA/RNA Molecule Synthetic oligonucleotide
acuuuuugguaacgguuautt

SEQ ID NO: 505 - DNA, 21 bp, Artificial Sequence
  Description of Artificial Sequence Synthetic oligonucleotide
  Description of Combined DNA/RNA Molecule Synthetic oligonucleotide
ggacuuuuugguaacgguutt

SEQ ID NO: 506 - DNA, 21 bp, Artificial Sequence
  Description of Artificial Sequence Synthetic oligonucleotide
  Description of Combined DNA/RNA Molecule Synthetic oligonucleotide
aggacuuuuugguaacggutt

SEQ ID NO: 507 - DNA, 21 bp, Artificial Sequence
  Description of Artificial Sequence Synthetic oligonucleotide
  Description of Combined DNA/RNA Molecule Synthetic oligonucleotide
ggucaggacuuuuugguaatt

SEQ ID NO: 508 - DNA, 21 bp, Artificial Sequence
  Description of Artificial Sequence Synthetic oligonucleotide
  Description of Combined DNA/RNA Molecule Synthetic oligonucleotide
guagguccaguuaaaaccutt

SEQ ID NO: 509 - DNA, 21 bp, Artificial Sequence
  Description of Artificial Sequence Synthetic oligonucleotide
  Description of Combined DNA/RNA Molecule Synthetic oligonucleotide
gguagguccaguuaaaacctt

SEQ ID NO: 510 - DNA, 21 bp, Artificial Sequence
  Description of Artificial Sequence Synthetic oligonucleotide
  Description of Combined DNA/RNA Molecule Synthetic oligonucleotide
ccagguagguccaguuaaatt

SEQ ID NO: 511 - DNA, 21 bp, Artificial Sequence
  Description of Artificial Sequence Synthetic oligonucleotide
  Description of Combined DNA/RNA Molecule Synthetic oligonucleotide
cccagguagguccaguuaatt

SEQ ID NO: 512 - DNA, 21 bp, Artificial Sequence
  Description of Artificial Sequence Synthetic oligonucleotide
  Description of Combined DNA/RNA Molecule Synthetic oligonucleotide
aaagggucccagguagguctt

SEQ ID NO: 513 - DNA, 21 bp, Artificial Sequence
  Description of Artificial Sequence Synthetic oligonucleotide
  Description of Combined DNA/RNA Molecule Synthetic oligonucleotide
agggugagcucaaacgucatt

SEQ ID NO: 514 - DNA, 21 bp, Artificial Sequence
  Description of Artificial Sequence Synthetic oligonucleotide
  Description of Combined DNA/RNA Molecule Synthetic oligonucleotide
aagggugagcucaaacguctt

SEQ ID NO: 515 - DNA, 21 bp, Artificial Sequence
  Description of Artificial Sequence Synthetic oligonucleotide
  Description of Combined DNA/RNA Molecule Synthetic oligonucleotide
uaucgaagggugagcucaatt

SEQ ID NO: 516 - DNA, 21 bp, Artificial Sequence
  Description of Artificial Sequence Synthetic oligonucleotide
  Description of Combined DNA/RNA Molecule Synthetic oligonucleotide
ggguaucgaagggugagcutt

SEQ ID NO: 517 - DNA, 21 bp, Artificial Sequence
  Description of Artificial Sequence Synthetic oligonucleotide
  Description of Combined DNA/RNA Molecule Synthetic oligonucleotide
accucugggggguaucgaatt

SEQ ID NO: 518 - DNA, 21 bp, Artificial Sequence
  Description of Artificial Sequence Synthetic oligonucleotide
  Description of Combined DNA/RNA Molecule Synthetic oligonucleotide
cuuaccucugggggguauctt

SEQ ID NO: 519 - DNA, 21 bp, Artificial Sequence
  Description of Artificial Sequence Synthetic oligonucleotide
  Description of Combined DNA/RNA Molecule Synthetic oligonucleotide
gguugugcucuaaggucuctt

SEQ ID NO: 520 - DNA, 21 bp, Artificial Sequence
  Description of Artificial Sequence Synthetic oligonucleotide
  Description of Combined DNA/RNA Molecule Synthetic oligonucleotide
uugguugugcucuaagguctt

SEQ ID NO: 521 - DNA, 21 bp, Artificial Sequence
  Description of Artificial Sequence Synthetic oligonucleotide
  Description of Combined DNA/RNA Molecule Synthetic oligonucleotide
ggagccacuccccacgcugtt

SEQ ID NO: 522 - DNA, 21 bp, Artificial Sequence
  Description of Artificial Sequence Synthetic oligonucleotide
  Description of Combined DNA/RNA Molecule Synthetic oligonucleotide
aggaacucauccgggggautt

SEQ ID NO: 523 - DNA, 21 bp, Artificial Sequence
  Description of Artificial Sequence Synthetic oligonucleotide
  Description of Combined DNA/RNA Molecule Synthetic oligonucleotide
gaagaggaacucauccgggtt

SEQ ID NO: 524 - DNA, 21 bp, Artificial Sequence
  Description of Artificial Sequence Synthetic oligonucleotide
  Description of Combined DNA/RNA Molecule Synthetic oligonucleotide
uggacaugcaggcgaccactt

SEQ ID NO: 525 - DNA, 21 bp, Artificial Sequence
  Description of Artificial Sequence Synthetic oligonucleotide
  Description of Combined DNA/RNA Molecule Synthetic oligonucleotide
uuauacuuguacaauagcatt

SEQ ID NO: 526 - DNA, 21 bp, Artificial Sequence
  Description of Artificial Sequence Synthetic oligonucleotide
  Description of Combined DNA/RNA Molecule Synthetic oligonucleotide
ugaucuuccagcggaccugtt

SEQ ID NO: 527 - DNA, 21 bp, Artificial Sequence
  Description of Artificial Sequence Synthetic oligonucleotide
  Description of Combined DNA/RNA Molecule Synthetic oligonucleotide
gaugaucuuccagcggacctt

SEQ ID NO: 528 - DNA, 21 bp, Artificial Sequence
  Description of Artificial Sequence Synthetic oligonucleotide
  Description of Combined DNA/RNA Molecule Synthetic oligonucleotide
ucgaugaucuuccagcggatt

SEQ ID NO: 529 - DNA, 21 bp, Artificial Sequence
  Description of Artificial Sequence Synthetic oligonucleotide
  Description of Combined DNA/RNA Molecule Synthetic oligonucleotide
cucgaugaucuuccagcggtt

SEQ ID NO: 530 - DNA, 21 bp, Artificial Sequence
  Description of Artificial Sequence Synthetic oligonucleotide
  Description of Combined DNA/RNA Molecule Synthetic oligonucleotide
ccucauagcucucgaugautt

SEQ ID NO: 531 - DNA, 21 bp, Artificial Sequence
  Description of Artificial Sequence Synthetic oligonucleotide
  Description of Combined DNA/RNA Molecule Synthetic oligonucleotide
gcccucauagcucucgaugtt

SEQ ID NO: 532 - DNA, 21 bp, Artificial Sequence
  Description of Artificial Sequence Synthetic oligonucleotide
  Description of Combined DNA/RNA Molecule Synthetic oligonucleotide
uauaacuguugcccucauatt

SEQ ID NO: 533 - DNA, 21 bp, Artificial Sequence
  Description of Artificial Sequence Synthetic oligonucleotide
  Description of Combined DNA/RNA Molecule Synthetic oligonucleotide
aaaguauaacuguugcccutt

SEQ ID NO: 534 - DNA, 21 bp, Artificial Sequence
  Description of Artificial Sequence Synthetic oligonucleotide
  Description of Combined DNA/RNA Molecule Synthetic oligonucleotide
ugaaaguauaacuguugcctt

SEQ ID NO: 535 - DNA, 21 bp, Artificial Sequence
  Description of Artificial Sequence Synthetic oligonucleotide
  Description of Combined DNA/RNA Molecule Synthetic oligonucleotide
guuguaaggcagcugcgugtt

SEQ ID NO: 536 - DNA, 21 bp, Artificial Sequence
  Description of Artificial Sequence Synthetic oligonucleotide
  Description of Combined DNA/RNA Molecule Synthetic oligonucleotide
gcagguuguuccgggggaatt

SEQ ID NO: 537 - DNA, 21 bp, Artificial Sequence
  Description of Artificial Sequence Synthetic oligonucleotide
  Description of Combined DNA/RNA Molecule Synthetic oligonucleotide
aaacugcagguuguuccggtt

SEQ ID NO: 538 - DNA, 21 bp, Artificial Sequence
  Description of Artificial Sequence Synthetic oligonucleotide
  Description of Combined DNA/RNA Molecule Synthetic oligonucleotide
cuccgagggucuuaccaaatt

SEQ ID NO: 539 - DNA, 21 bp, Artificial Sequence
  Description of Artificial Sequence Synthetic oligonucleotide
  Description of Combined DNA/RNA Molecule Synthetic oligonucleotide
agcuccgagggucuuaccatt

SEQ ID NO: 540 - DNA, 21 bp, Artificial Sequence
  Description of Artificial Sequence Synthetic oligonucleotide
  Description of Combined DNA/RNA Molecule Synthetic oligonucleotide
gcaugggccguggacuucatt

SEQ ID NO: 541 - DNA, 21 bp, Artificial Sequence
  Description of Artificial Sequence Synthetic oligonucleotide
  Description of Combined DNA/RNA Molecule Synthetic oligonucleotide
agcaugggccguggacuuctt

SEQ ID NO: 542 - DNA, 21 bp, Artificial Sequence
  Description of Artificial Sequence Synthetic oligonucleotide
  Description of Combined DNA/RNA Molecule Synthetic oligonucleotide
ccgugaugaccaguacaggtt

SEQ ID NO: 543 - DNA, 21 bp, Artificial Sequence
  Description of Artificial Sequence Synthetic oligonucleotide
  Description of Combined DNA/RNA Molecule Synthetic oligonucleotide
uccgugaugaccaguacagtt

SEQ ID NO: 544 - DNA, 21 bp, Artificial Sequence
  Description of Artificial Sequence Synthetic oligonucleotide
  Description of Combined DNA/RNA Molecule Synthetic oligonucleotide
cuccgugaugaccaguacatt

SEQ ID NO: 545 - DNA, 21 bp, Artificial Sequence
  Description of Artificial Sequence Synthetic oligonucleotide
  Description of Combined DNA/RNA Molecule Synthetic oligonucleotide
uacuccgugaugaccaguatt

SEQ ID NO: 546 - DNA, 21 bp, Artificial Sequence
  Description of Artificial Sequence Synthetic oligonucleotide
  Description of Combined DNA/RNA Molecule Synthetic oligonucleotide
acaguacuccgugaugacctt

SEQ ID NO: 547 - DNA, 21 bp, Artificial Sequence
  Description of Artificial Sequence Synthetic oligonucleotide
  Description of Combined DNA/RNA Molecule Synthetic oligonucleotide
agcaacaguacuccgugautt

SEQ ID NO: 548 - DNA, 21 bp, Artificial Sequence
  Description of Artificial Sequence Synthetic oligonucleotide
  Description of Combined DNA/RNA Molecule Synthetic oligonucleotide
uagcaacaguacuccgugatt

SEQ ID NO: 549 - DNA, 21 bp, Artificial Sequence
  Description of Artificial Sequence Synthetic oligonucleotide
  Description of Combined DNA/RNA Molecule Synthetic oligonucleotide
cauagcaacaguacuccgutt

SEQ ID NO: 550 - DNA, 21 bp, Artificial Sequence
  Description of Artificial Sequence Synthetic oligonucleotide
  Description of Combined DNA/RNA Molecule Synthetic oligonucleotide
ucgccauagcaacaguacutt

SEQ ID NO: 551 - DNA, 21 bp, Artificial Sequence
  Description of Artificial Sequence Synthetic oligonucleotide
  Description of Combined DNA/RNA Molecule Synthetic oligonucleotide
gcaggucgccauagcaacatt

SEQ ID NO: 552 - DNA, 21 bp, Artificial Sequence
  Description of Artificial Sequence Synthetic oligonucleotide
  Description of Combined DNA/RNA Molecule Synthetic oligonucleotide
uugagcaggucgccauagctt

SEQ ID NO: 553 - DNA, 21 bp, Artificial Sequence
  Description of Artificial Sequence Synthetic oligonucleotide
  Description of Combined DNA/RNA Molecule Synthetic oligonucleotide
aaguugagcaggucgccautt

SEQ ID NO: 554 - DNA, 21 bp, Artificial Sequence
  Description of Artificial Sequence Synthetic oligonucleotide
  Description of Combined DNA/RNA Molecule Synthetic oligonucleotide
ccuucgcagaaaguugagctt

SEQ ID NO: 555 - DNA, 21 bp, Artificial Sequence
  Description of Artificial Sequence Synthetic oligonucleotide
  Description of Combined DNA/RNA Molecule Synthetic oligonucleotide
cuuccuucgcagaaaguugtt

SEQ ID NO: 556 - DNA, 21 bp, Artificial Sequence
  Description of Artificial Sequence Synthetic oligonucleotide
  Description of Combined DNA/RNA Molecule Synthetic oligonucleotide
ucagccuuccuucgcagaatt

SEQ ID NO: 557 - DNA, 21 bp, Artificial Sequence
  Description of Artificial Sequence Synthetic oligonucleotide
  Description of Combined DNA/RNA Molecule Synthetic oligonucleotide
cauagguguccacacccugtt

SEQ ID NO: 558 - DNA, 21 bp, Artificial Sequence
  Description of Artificial Sequence Synthetic oligonucleotide
  Description of Combined DNA/RNA Molecule Synthetic oligonucleotide
ccguccauccuccuugucctt

SEQ ID NO: 559 - DNA, 21 bp, Artificial Sequence
  Description of Artificial Sequence Synthetic oligonucleotide
  Description of Combined DNA/RNA Molecule Synthetic oligonucleotide
ggaugcaauucuuggaagctt

SEQ ID NO: 560 - DNA, 21 bp, Artificial Sequence
  Description of Artificial Sequence Synthetic oligonucleotide
  Description of Combined DNA/RNA Molecule Synthetic oligonucleotide
ucccuagccagcccgaagutt

SEQ ID NO: 561 - DNA, 21 bp, Artificial Sequence
  Description of Artificial Sequence Synthetic oligonucleotide
  Description of Combined DNA/RNA Molecule Synthetic oligonucleotide
ucaugaugucccuagccagtt

SEQ ID NO: 562 - DNA, 21 bp, Artificial Sequence
  Description of Artificial Sequence Synthetic oligonucleotide
  Description of Combined DNA/RNA Molecule Synthetic oligonucleotide
auucaugaugucccuagcctt

SEQ ID NO: 563 - DNA, 21 bp, Artificial Sequence
  Description of Artificial Sequence Synthetic oligonucleotide
  Description of Combined DNA/RNA Molecule Synthetic oligonucleotide
ccguguagacacagucaaatt

SEQ ID NO: 564 - DNA, 21 bp, Artificial Sequence
  Description of Artificial Sequence Synthetic oligonucleotide
  Description of Combined DNA/RNA Molecule Synthetic oligonucleotide
aaccguguagacacagucatt

SEQ ID NO: 565 - DNA, 21 bp, Artificial Sequence
  Description of Artificial Sequence Synthetic oligonucleotide
  Description of Combined DNA/RNA Molecule Synthetic oligonucleotide
ugaaccguguagacacagutt

SEQ ID NO: 566 - DNA, 21 bp, Artificial Sequence
  Description of Artificial Sequence Synthetic oligonucleotide
  Description of Combined DNA/RNA Molecule Synthetic oligonucleotide
cucugaaccguguagacactt

SEQ ID NO: 567 - DNA, 21 bp, Artificial Sequence
  Description of Artificial Sequence Synthetic oligonucleotide
  Description of Combined DNA/RNA Molecule Synthetic oligonucleotide
gucgcucugaaccguguagtt

SEQ ID NO: 568 - DNA, 21 bp, Artificial Sequence
  Description of Artificial Sequence Synthetic oligonucleotide
  Description of Combined DNA/RNA Molecule Synthetic oligonucleotide
gacgucgcucugaaccgugtt

SEQ ID NO: 569 - DNA, 21 bp, Artificial Sequence
  Description of Artificial Sequence Synthetic oligonucleotide
  Description of Combined DNA/RNA Molecule Synthetic oligonucleotide
ccagacgucgcucugaacctt

SEQ ID NO: 570 - DNA, 21 bp, Artificial Sequence
  Description of Artificial Sequence Synthetic oligonucleotide
  Description of Combined DNA/RNA Molecule Synthetic oligonucleotide
gaccagacgucgcucugaatt

SEQ ID NO: 571 - DNA, 21 bp, Artificial Sequence
  Description of Artificial Sequence Synthetic oligonucleotide
  Description of Combined DNA/RNA Molecule Synthetic oligonucleotide
gguagggauucagcccaagtt

SEQ ID NO: 572 - DNA, 21 bp, Artificial Sequence
  Description of Artificial Sequence Synthetic oligonucleotide
  Description of Combined DNA/RNA Molecule Synthetic oligonucleotide
uuauagaacuugcuguucatt

SEQ ID NO: 573 - DNA, 21 bp, Artificial Sequence
  Description of Artificial Sequence Synthetic oligonucleotide
  Description of Combined DNA/RNA Molecule Synthetic oligonucleotide
accaguuuauagaacuugctt

SEQ ID NO: 574 - DNA, 21 bp, Artificial Sequence
  Description of Artificial Sequence Synthetic oligonucleotide
  Description of Combined DNA/RNA Molecule Synthetic oligonucleotide
augaugcuguauauauucutt

SEQ ID NO: 575 - DNA, 21 bp, Artificial Sequence
  Description of Artificial Sequence Synthetic oligonucleotide
  Description of Combined DNA/RNA Molecule Synthetic oligonucleotide
ugguauagucccgcucucutt

SEQ ID NO: 576 - DNA, 21 bp, Artificial Sequence
  Description of Artificial Sequence Synthetic oligonucleotide
  Description of Combined DNA/RNA Molecule Synthetic oligonucleotide
uugguauagucccgcucuctt

SEQ ID NO: 577 - DNA, 21 bp, Artificial Sequence
  Description of Artificial Sequence Synthetic oligonucleotide
  Description of Combined DNA/RNA Molecule Synthetic oligonucleotide
auugguauagucccgcucutt

SEQ ID NO: 578 - DNA, 21 bp, Artificial Sequence
  Description of Artificial Sequence Synthetic oligonucleotide
  Description of Combined DNA/RNA Molecule Synthetic oligonucleotide
cagauugguauagucccgctt

SEQ ID NO: 579 - DNA, 21 bp, Artificial Sequence
  Description of Artificial Sequence Synthetic oligonucleotide
  Description of Combined DNA/RNA Molecule Synthetic oligonucleotide
cggcagauugguauagucctt

SEQ ID NO: 580 - DNA, 21 bp, Artificial Sequence
  Description of Artificial Sequence Synthetic oligonucleotide
  Description of Combined DNA/RNA Molecule Synthetic oligonucleotide
uauccccuugcucgcagcatt

SEQ ID NO: 581 - DNA, 21 bp, Artificial Sequence
  Description of Artificial Sequence Synthetic oligonucleotide
  Description of Combined DNA/RNA Molecule Synthetic oligonucleotide
gcgauauccccuugcucgctt

SEQ ID NO: 582 - DNA, 21 bp, Artificial Sequence
  Description of Artificial Sequence Synthetic oligonucleotide
  Description of Combined DNA/RNA Molecule Synthetic oligonucleotide
ggcgauauccccuugcucgtt

SEQ ID NO: 583 - DNA, 21 bp, Artificial Sequence
  Description of Artificial Sequence Synthetic oligonucleotide
  Description of Combined DNA/RNA Molecule Synthetic oligonucleotide
gcaagggcugggcgauauctt

SEQ ID NO: 584 - DNA, 21 bp, Artificial Sequence
  Description of Artificial Sequence Synthetic oligonucleotide
  Description of Combined DNA/RNA Molecule Synthetic oligonucleotide
aacugauaguuguugggcutt

SEQ ID NO: 585 - DNA, 21 bp, Artificial Sequence
  Description of Artificial Sequence Synthetic oligonucleotide
  Description of Combined DNA/RNA Molecule Synthetic oligonucleotide
gaacugauaguuguugggctt

SEQ ID NO: 586 - DNA, 21 bp, Artificial Sequence
  Description of Artificial Sequence Synthetic oligonucleotide
  Description of Combined DNA/RNA Molecule Synthetic oligonucleotide
agaacugauaguuguugggtt

SEQ ID NO: 587 - DNA, 21 bp, Artificial Sequence
  Description of Artificial Sequence Synthetic oligonucleotide
  Description of Combined DNA/RNA Molecule Synthetic oligonucleotide
gaccgaaggcagaguuugutt

SEQ ID NO: 588 - DNA, 21 bp, Artificial Sequence
  Description of Artificial Sequence Synthetic oligonucleotide
  Description of Combined DNA/RNA Molecule Synthetic oligonucleotide
ugaaaugaccgaaggcagatt

SEQ ID NO: 589 - DNA, 21 bp, Artificial Sequence
  Description of Artificial Sequence Synthetic oligonucleotide
  Description of Combined DNA/RNA Molecule Synthetic oligonucleotide
gagugaaaugaccgaaggctt

SEQ ID NO: 590 - DNA, 21 bp, Artificial Sequence
  Description of Artificial Sequence Synthetic oligonucleotide
  Description of Combined DNA/RNA Molecule Synthetic oligonucleotide
uguugagugaaaugaccgatt

SEQ ID NO: 591 - DNA, 21 bp, Artificial Sequence
  Description of Artificial Sequence Synthetic oligonucleotide
  Description of Combined DNA/RNA Molecule Synthetic oligonucleotide
acgaggccaacaccaugagtt

SEQ ID NO: 592 - DNA, 21 bp, Artificial Sequence
  Description of Artificial Sequence Synthetic oligonucleotide
  Description of Combined DNA/RNA Molecule Synthetic oligonucleotide
cacgaggccaacaccaugatt

SEQ ID NO: 593 - DNA, 21 bp, Artificial Sequence
  Description of Artificial Sequence Synthetic oligonucleotide
  Description of Combined DNA/RNA Molecule Synthetic oligonucleotide
acacgaggccaacaccaugtt

SEQ ID NO: 594 - DNA, 21 bp, Artificial Sequence
  Description of Artificial Sequence Synthetic oligonucleotide
  Description of Combined DNA/RNA Molecule Synthetic oligonucleotide
caaacacgaggccaacacctt

SEQ ID NO: 595 - DNA, 21 bp, Artificial Sequence
  Description of Artificial Sequence Synthetic oligonucleotide
  Description of Combined DNA/RNA Molecule Synthetic oligonucleotide
aguuggcauagcaaacacgtt

SEQ ID NO: 596 - DNA, 21 bp, Artificial Sequence
  Description of Artificial Sequence Synthetic oligonucleotide
  Description of Combined DNA/RNA Molecule Synthetic oligonucleotide
acacccccaacuuuuagcutt

SEQ ID NO: 597 - DNA, 21 bp, Artificial Sequence
  Description of Artificial Sequence Synthetic oligonucleotide
  Description of Combined DNA/RNA Molecule Synthetic oligonucleotide
uuaggaugagucagcuuggtt

SEQ ID NO: 598 - DNA, 21 bp, Artificial Sequence
  Description of Artificial Sequence Synthetic oligonucleotide
  Description of Combined DNA/RNA Molecule Synthetic oligonucleotide
uaguuaggaugagucagcutt

SEQ ID NO: 599 - DNA, 21 bp, Artificial Sequence
  Description of Artificial Sequence Synthetic oligonucleotide
  Description of Combined DNA/RNA Molecule Synthetic oligonucleotide
guuaguuaggaugagucagtt

SEQ ID NO: 600 - DNA, 21 bp, Artificial Sequence
  Description of Artificial Sequence Synthetic oligonucleotide
  Description of Combined DNA/RNA Molecule Synthetic oligonucleotide
uguuaguuaggaugagucatt

SEQ ID NO: 601 - DNA, 21 bp, Artificial Sequence
  Description of Artificial Sequence Synthetic oligonucleotide
  Description of Combined DNA/RNA Molecule Synthetic oligonucleotide
cuguuaguuaggaugaguctt

SEQ ID NO: 602 - DNA, 21 bp, Artificial Sequence
  Description of Artificial Sequence Synthetic oligonucleotide
  Description of Combined DNA/RNA Molecule Synthetic oligonucleotide
gcgugacuguuaguuaggatt

SEQ ID NO: 603 - DNA, 21 bp, Artificial Sequence
  Description of Artificial Sequence Synthetic oligonucleotide
  Description of Combined DNA/RNA Molecule Synthetic oligonucleotide
ggcgugacuguuaguuaggtt

SEQ ID NO: 604 - DNA, 21 bp, Artificial Sequence
  Description of Artificial Sequence Synthetic oligonucleotide
  Description of Combined DNA/RNA Molecule Synthetic oligonucleotide
gcuguuaguuuaauguggatt

SEQ ID NO: 605 - DNA, 21 bp, Artificial Sequence
  Description of Artificial Sequence Synthetic oligonucleotide
  Description of Combined DNA/RNA Molecule Synthetic oligonucleotide
ugcuguuaguuuaauguggtt

SEQ ID NO: 606 - DNA, 21 bp, Artificial Sequence
  Description of Artificial Sequence Synthetic oligonucleotide
  Description of Combined DNA/RNA Molecule Synthetic oligonucleotide
caaggacgcauuuccccagtt

SEQ ID NO: 607 - DNA, 21 bp, Artificial Sequence
  Description of Artificial Sequence Synthetic oligonucleotide
  Description of Combined DNA/RNA Molecule Synthetic oligonucleotide
aaggacgcauuuccccagutt

SEQ ID NO: 608 - DNA, 21 bp, Artificial Sequence
  Description of Artificial Sequence Synthetic oligonucleotide
  Description of Combined DNA/RNA Molecule Synthetic oligonucleotide
ggacgcauuuccccaguactt

SEQ ID NO: 609 - DNA, 21 bp, Artificial Sequence
  Description of Artificial Sequence Synthetic oligonucleotide
  Description of Combined DNA/RNA Molecule Synthetic oligonucleotide
gacgcauuuccccaguacatt

SEQ ID NO: 610 - DNA, 21 bp, Artificial Sequence
  Description of Artificial Sequence Synthetic oligonucleotide
  Description of Combined DNA/RNA Molecule Synthetic oligonucleotide
acgcauuuccccaguacautt

SEQ ID NO: 611 - DNA, 21 bp, Artificial Sequence
  Description of Artificial Sequence Synthetic oligonucleotide
  Description of Combined DNA/RNA Molecule Synthetic oligonucleotide
cgcauuuccccaguacauctt

SEQ ID NO: 612 - DNA, 21 bp, Artificial Sequence
  Description of Artificial Sequence Synthetic oligonucleotide
  Description of Combined DNA/RNA Molecule Synthetic oligonucleotide
cauuuccccaguacauccatt

SEQ ID NO: 613 - DNA, 21 bp, Artificial Sequence
  Description of Artificial Sequence Synthetic oligonucleotide
  Description of Combined DNA/RNA Molecule Synthetic oligonucleotide
auuuccccaguacauccactt

SEQ ID NO: 614 - DNA, 21 bp, Artificial Sequence
  Description of Artificial Sequence Synthetic oligonucleotide
  Description of Combined DNA/RNA Molecule Synthetic oligonucleotide
uuuccccaguacauccacatt

SEQ ID NO: 615 - DNA, 21 bp, Artificial Sequence
  Description of Artificial Sequence Synthetic oligonucleotide
  Description of Combined DNA/RNA Molecule Synthetic oligonucleotide
uccccaguacauccacaactt

SEQ ID NO: 616 - DNA, 21 bp, Artificial Sequence
  Description of Artificial Sequence Synthetic oligonucleotide
  Description of Combined DNA/RNA Molecule Synthetic oligonucleotide
caguacauccacaacaugctt

SEQ ID NO: 617 - DNA, 21 bp, Artificial Sequence
  Description of Artificial Sequence Synthetic oligonucleotide
  Description of Combined DNA/RNA Molecule Synthetic oligonucleotide
guacauccacaacaugcugtt

SEQ ID NO: 618 - DNA, 21 bp, Artificial Sequence
  Description of Artificial Sequence Synthetic oligonucleotide
  Description of Combined DNA/RNA Molecule Synthetic oligonucleotide
acauccacaacaugcuguctt

SEQ ID NO: 619 - DNA, 21 bp, Artificial Sequence
  Description of Artificial Sequence Synthetic oligonucleotide
  Description of Combined DNA/RNA Molecule Synthetic oligonucleotide
augcuguccacaucucguutt

SEQ ID NO: 620 - DNA, 21 bp, Artificial Sequence
  Description of Artificial Sequence Synthetic oligonucleotide
  Description of Combined DNA/RNA Molecule Synthetic oligonucleotide
ugcuguccacaucucguuctt

SEQ ID NO: 621 - DNA, 21 bp, Artificial Sequence
  Description of Artificial Sequence Synthetic oligonucleotide
  Description of Combined DNA/RNA Molecule Synthetic oligonucleotide
gcuguccacaucucguucutt

SEQ ID NO: 622 - DNA, 21 bp, Artificial Sequence
  Description of Artificial Sequence Synthetic oligonucleotide
  Description of Combined DNA/RNA Molecule Synthetic oligonucleotide
cuguccacaucucguucuctt

SEQ ID NO: 623 - DNA, 21 bp, Artificial Sequence
  Description of Artificial Sequence Synthetic oligonucleotide
  Description of Combined DNA/RNA Molecule Synthetic oligonucleotide
guccacaucucguucucggtt

SEQ ID NO: 624 - DNA, 21 bp, Artificial Sequence
  Description of Artificial Sequence Synthetic oligonucleotide
  Description of Combined DNA/RNA Molecule Synthetic oligonucleotide
uccacaucucguucucggutt

SEQ ID NO: 625 - DNA, 21 bp, Artificial Sequence
  Description of Artificial Sequence Synthetic oligonucleotide
  Description of Combined DNA/RNA Molecule Synthetic oligonucleotide
ccacaucucguucucgguutt

SEQ ID NO: 626 - DNA, 21 bp, Artificial Sequence
  Description of Artificial Sequence Synthetic oligonucleotide
  Description of Combined DNA/RNA Molecule Synthetic oligonucleotide
cacaucucguucucgguuutt

SEQ ID NO: 627 - DNA, 21 bp, Artificial Sequence
  Description of Artificial Sequence Synthetic oligonucleotide
  Description of Combined DNA/RNA Molecule Synthetic oligonucleotide
acaucucguucucgguuuatt

SEQ ID NO: 628 - DNA, 21 bp, Artificial Sequence
  Description of Artificial Sequence Synthetic oligonucleotide
  Description of Combined DNA/RNA Molecule Synthetic oligonucleotide
caucucguucucgguuuautt

SEQ ID NO: 629 - DNA, 21 bp, Artificial Sequence
  Description of Artificial Sequence Synthetic oligonucleotide
  Description of Combined DNA/RNA Molecule Synthetic oligonucleotide
ucucguucucgguuuaucatt

SEQ ID NO: 630 - DNA, 21 bp, Artificial Sequence
  Description of Artificial Sequence Synthetic oligonucleotide
  Description of Combined DNA/RNA Molecule Synthetic oligonucleotide
cucguucucgguuuaucagtt

SEQ ID NO: 631 - DNA, 21 bp, Artificial Sequence
  Description of Artificial Sequence Synthetic oligonucleotide
  Description of Combined DNA/RNA Molecule Synthetic oligonucleotide
ucguucucgguuuaucagatt

SEQ ID NO: 632 - DNA, 21 bp, Artificial Sequence
  Description of Artificial Sequence Synthetic oligonucleotide
  Description of Combined DNA/RNA Molecule Synthetic oligonucleotide
cguucucgguuuaucagaatt

SEQ ID NO: 633 - DNA, 21 bp, Artificial Sequence
  Description of Artificial Sequence Synthetic oligonucleotide
  Description of Combined DNA/RNA Molecule Synthetic oligonucleotide
uucucgguuuaucagaaautt

SEQ ID NO: 634 - DNA, 21 bp, Artificial Sequence
  Description of Artificial Sequence Synthetic oligonucleotide
  Description of Combined DNA/RNA Molecule Synthetic oligonucleotide
ucucgguuuaucagaaauatt

SEQ ID NO: 635 - DNA, 21 bp, Artificial Sequence
  Description of Artificial Sequence Synthetic oligonucleotide
  Description of Combined DNA/RNA Molecule Synthetic oligonucleotide
cucgguuuaucagaaauactt

SEQ ID NO: 636 - DNA, 21 bp, Artificial Sequence
  Description of Artificial Sequence Synthetic oligonucleotide
  Description of Combined DNA/RNA Molecule Synthetic oligonucleotide
ucgguuuaucagaaauacctt

SEQ ID NO: 637 - DNA, 21 bp, Artificial Sequence
  Description of Artificial Sequence Synthetic oligonucleotide
  Description of Combined DNA/RNA Molecule Synthetic oligonucleotide
uuuaucagaaauaccaacgtt

SEQ ID NO: 638 - DNA, 21 bp, Artificial Sequence
  Description of Artificial Sequence Synthetic oligonucleotide
  Description of Combined DNA/RNA Molecule Synthetic oligonucleotide
agagcggugaagaagucactt

SEQ ID NO: 639 - DNA, 21 bp, Artificial Sequence
  Description of Artificial Sequence Synthetic oligonucleotide
  Description of Combined DNA/RNA Molecule Synthetic oligonucleotide
cggugaagaagucaccacctt

SEQ ID NO: 640 - DNA, 21 bp, Artificial Sequence
  Description of Artificial Sequence Synthetic oligonucleotide
  Description of Combined DNA/RNA Molecule Synthetic oligonucleotide
aagucaccaccuuuuuugatt

SEQ ID NO: 641 - DNA, 21 bp, Artificial Sequence
  Description of Artificial Sequence Synthetic oligonucleotide
  Description of Combined DNA/RNA Molecule Synthetic oligonucleotide
ucaccaccuuuuuugauuatt

SEQ ID NO: 642 - DNA, 21 bp, Artificial Sequence
  Description of Artificial Sequence Synthetic oligonucleotide
  Description of Combined DNA/RNA Molecule Synthetic oligonucleotide
caccaccuuuuuugauuautt

SEQ ID NO: 643 - DNA, 21 bp, Artificial Sequence
  Description of Artificial Sequence Synthetic oligonucleotide
  Description of Combined DNA/RNA Molecule Synthetic oligonucleotide
accaccuuuuuugauuaugtt

SEQ ID NO: 644 - DNA, 21 bp, Artificial Sequence
  Description of Artificial Sequence Synthetic oligonucleotide
  Description of Combined DNA/RNA Molecule Synthetic oligonucleotide
uuuugauuaugauuacggutt

SEQ ID NO: 645 - DNA, 21 bp, Artificial Sequence
  Description of Artificial Sequence Synthetic oligonucleotide
  Description of Combined DNA/RNA Molecule Synthetic oligonucleotide
ugauuaugauuacggugcutt

SEQ ID NO: 646 - DNA, 21 bp, Artificial Sequence
  Description of Artificial Sequence Synthetic oligonucleotide
  Description of Combined DNA/RNA Molecule Synthetic oligonucleotide
uuaugauuacggugcuccctt

SEQ ID NO: 647 - DNA, 21 bp, Artificial Sequence
  Description of Artificial Sequence Synthetic oligonucleotide
  Description of Combined DNA/RNA Molecule Synthetic oligonucleotide
uaugauuacggugcucccutt

SEQ ID NO: 648 - DNA, 21 bp, Artificial Sequence
  Description of Artificial Sequence Synthetic oligonucleotide
  Description of Combined DNA/RNA Molecule Synthetic oligonucleotide
ugauuacggugcucccugutt

SEQ ID NO: 649 - DNA, 21 bp, Artificial Sequence
  Description of Artificial Sequence Synthetic oligonucleotide
  Description of Combined DNA/RNA Molecule Synthetic oligonucleotide
gauuacggugcucccuguctt

SEQ ID NO: 650 - DNA, 21 bp, Artificial Sequence
  Description of Artificial Sequence Synthetic oligonucleotide
  Description of Combined DNA/RNA Molecule Synthetic oligonucleotide
auuacggugcucccugucatt

SEQ ID NO: 651 - DNA, 21 bp, Artificial Sequence
  Description of Artificial Sequence Synthetic oligonucleotide
  Description of Combined DNA/RNA Molecule Synthetic oligonucleotide
uuacggugcucccugucautt

SEQ ID NO: 652 - DNA, 21 bp, Artificial Sequence
  Description of Artificial Sequence Synthetic oligonucleotide
  Description of Combined DNA/RNA Molecule Synthetic oligonucleotide
acggugcucccugucauaatt

SEQ ID NO: 653 - DNA, 21 bp, Artificial Sequence
  Description of Artificial Sequence Synthetic oligonucleotide
  Description of Combined DNA/RNA Molecule Synthetic oligonucleotide
gugcucccugucauaaauutt

SEQ ID NO: 654 - DNA, 21 bp, Artificial Sequence
  Description of Artificial Sequence Synthetic oligonucleotide
  Description of Combined DNA/RNA Molecule Synthetic oligonucleotide
ugcucccugucauaaauuutt

SEQ ID NO: 655 - DNA, 21 bp, Artificial Sequence
  Description of Artificial Sequence Synthetic oligonucleotide
  Description of Combined DNA/RNA Molecule Synthetic oligonucleotide
cucccugucauaaauuugatt

SEQ ID NO: 656 - DNA, 21 bp, Artificial Sequence
  Description of Artificial Sequence Synthetic oligonucleotide
  Description of Combined DNA/RNA Molecule Synthetic oligonucleotide
ccugucauaaauuugacgutt

SEQ ID NO: 657 - DNA, 21 bp, Artificial Sequence
  Description of Artificial Sequence Synthetic oligonucleotide
  Description of Combined DNA/RNA Molecule Synthetic oligonucleotide
ugucauaaauuugacgugatt

SEQ ID NO: 658 - DNA, 21 bp, Artificial Sequence
  Description of Artificial Sequence Synthetic oligonucleotide
  Description of Combined DNA/RNA Molecule Synthetic oligonucleotide
gucauaaauuugacgugaatt

SEQ ID NO: 659 - DNA, 21 bp, Artificial Sequence
  Description of Artificial Sequence Synthetic oligonucleotide
  Description of Combined DNA/RNA Molecule Synthetic oligonucleotide
ucauaaauuugacgugaagtt

SEQ ID NO: 660 - DNA, 21 bp, Artificial Sequence
  Description of Artificial Sequence Synthetic oligonucleotide
  Description of Combined DNA/RNA Molecule Synthetic oligonucleotide
auaaauuugacgugaagcatt

SEQ ID NO: 661 - DNA, 21 bp, Artificial Sequence
  Description of Artificial Sequence Synthetic oligonucleotide
  Description of Combined DNA/RNA Molecule Synthetic oligonucleotide
aauuugacgugaagcaaautt

SEQ ID NO: 662 - DNA, 21 bp, Artificial Sequence
  Description of Artificial Sequence Synthetic oligonucleotide
  Description of Combined DNA/RNA Molecule Synthetic oligonucleotide
auuugacgugaagcaaauutt

SEQ ID NO: 663 - DNA, 21 bp, Artificial Sequence
  Description of Artificial Sequence Synthetic oligonucleotide
  Description of Combined DNA/RNA Molecule Synthetic oligonucleotide
uuugacgugaagcaaauugtt

SEQ ID NO: 664 - DNA, 21 bp, Artificial Sequence
  Description of Artificial Sequence Synthetic oligonucleotide
  Description of Combined DNA/RNA Molecule Synthetic oligonucleotide
uugacgugaagcaaauuggtt

SEQ ID NO: 665 - DNA, 21 bp, Artificial Sequence
  Description of Artificial Sequence Synthetic oligonucleotide
  Description of Combined DNA/RNA Molecule Synthetic oligonucleotide
ugacgugaagcaaauugggtt

SEQ ID NO: 666 - DNA, 21 bp, Artificial Sequence
  Description of Artificial Sequence Synthetic oligonucleotide
  Description of Combined DNA/RNA Molecule Synthetic oligonucleotide
gacgugaagcaaauuggggtt

SEQ ID NO: 667 - DNA, 21 bp, Artificial Sequence
  Description of Artificial Sequence Synthetic oligonucleotide
  Description of Combined DNA/RNA Molecule Synthetic oligonucleotide
acgugaagcaaauuggggctt

SEQ ID NO: 668 - DNA, 21 bp, Artificial Sequence
  Description of Artificial Sequence Synthetic oligonucleotide
  Description of Combined DNA/RNA Molecule Synthetic oligonucleotide
cgugaagcaaauuggggcctt

SEQ ID NO: 669 - DNA, 21 bp, Artificial Sequence
  Description of Artificial Sequence Synthetic oligonucleotide
  Description of Combined DNA/RNA Molecule Synthetic oligonucleotide
gaagcaaauuggggcccaatt

SEQ ID NO: 670 - DNA, 21 bp, Artificial Sequence
  Description of Artificial Sequence Synthetic oligonucleotide
  Description of Combined DNA/RNA Molecule Synthetic oligonucleotide
aagcaaauuggggcccaactt

SEQ ID NO: 671 - DNA, 21 bp, Artificial Sequence
  Description of Artificial Sequence Synthetic oligonucleotide
  Description of Combined DNA/RNA Molecule Synthetic oligonucleotide
agcaaauuggggcccaacutt

SEQ ID NO: 672 - DNA, 21 bp, Artificial Sequence
  Description of Artificial Sequence Synthetic oligonucleotide
  Description of Combined DNA/RNA Molecule Synthetic oligonucleotide
gcaaauuggggcccaacuctt

SEQ ID NO: 673 - DNA, 21 bp, Artificial Sequence
  Description of Artificial Sequence Synthetic oligonucleotide
  Description of Combined DNA/RNA Molecule Synthetic oligonucleotide
ugcaaaaagcugaagugcutt

SEQ ID NO: 674 - DNA, 21 bp, Artificial Sequence
  Description of Artificial Sequence Synthetic oligonucleotide
  Description of Combined DNA/RNA Molecule Synthetic oligonucleotide
aaagcugaagugcuugacutt

SEQ ID NO: 675 - DNA, 21 bp, Artificial Sequence
  Description of Artificial Sequence Synthetic oligonucleotide
  Description of Combined DNA/RNA Molecule Synthetic oligonucleotide
ugcuugacugacauuuacctt

SEQ ID NO: 676 - DNA, 21 bp, Artificial Sequence
  Description of Artificial Sequence Synthetic oligonucleotide
  Description of Combined DNA/RNA Molecule Synthetic oligonucleotide
cuugacugacauuuaccugtt

SEQ ID NO: 677 - DNA, 21 bp, Artificial Sequence
  Description of Artificial Sequence Synthetic oligonucleotide
  Description of Combined DNA/RNA Molecule Synthetic oligonucleotide
ugcuuuuucuuauuacucutt

SEQ ID NO: 678 - DNA, 21 bp, Artificial Sequence
  Description of Artificial Sequence Synthetic oligonucleotide
  Description of Combined DNA/RNA Molecule Synthetic oligonucleotide
uucuuauuacucucccauutt

SEQ ID NO: 679 - DNA, 21 bp, Artificial Sequence
  Description of Artificial Sequence Synthetic oligonucleotide
  Description of Combined DNA/RNA Molecule Synthetic oligonucleotide
cucccauugugggcucacutt

SEQ ID NO: 680 - DNA, 21 bp, Artificial Sequence
  Description of Artificial Sequence Synthetic oligonucleotide
  Description of Combined DNA/RNA Molecule Synthetic oligonucleotide
cccauugugggcucacucutt

SEQ ID NO: 681 - DNA, 21 bp, Artificial Sequence
  Description of Artificial Sequence Synthetic oligonucleotide
  Description of Combined DNA/RNA Molecule Synthetic oligonucleotide
cacucugcugcaaaugagutt

SEQ ID NO: 682 - DNA, 21 bp, Artificial Sequence
  Description of Artificial Sequence Synthetic oligonucleotide
  Description of Combined DNA/RNA Molecule Synthetic oligonucleotide
acucugcugcaaaugagugtt

SEQ ID NO: 683 - DNA, 21 bp, Artificial Sequence
  Description of Artificial Sequence Synthetic oligonucleotide
  Description of Combined DNA/RNA Molecule Synthetic oligonucleotide
agugggucuuugggaaugctt

SEQ ID NO: 684 - DNA, 21 bp, Artificial Sequence
  Description of Artificial Sequence Synthetic oligonucleotide
  Description of Combined DNA/RNA Molecule Synthetic oligonucleotide
gugggucuuugggaaugcatt

SEQ ID NO: 685 - DNA, 21 bp, Artificial Sequence
  Description of Artificial Sequence Synthetic oligonucleotide
  Description of Combined DNA/RNA Molecule Synthetic oligonucleotide
gggucuuugggaaugcaautt

SEQ ID NO: 686 - DNA, 21 bp, Artificial Sequence
  Description of Artificial Sequence Synthetic oligonucleotide
  Description of Combined DNA/RNA Molecule Synthetic oligonucleotide
uugggaaugcaaugugcaatt

SEQ ID NO: 687 - DNA, 21 bp, Artificial Sequence
  Description of Artificial Sequence Synthetic oligonucleotide
  Description of Combined DNA/RNA Molecule Synthetic oligonucleotide
gugcaaauuauucacagggtt

SEQ ID NO: 688 - DNA, 21 bp, Artificial Sequence
  Description of Artificial Sequence Synthetic oligonucleotide
  Description of Combined DNA/RNA Molecule Synthetic oligonucleotide
cacagggcuguaucacauctt

SEQ ID NO: 689 - DNA, 21 bp, Artificial Sequence
  Description of Artificial Sequence Synthetic oligonucleotide
  Description of Combined DNA/RNA Molecule Synthetic oligonucleotide
acagggcuguaucacaucgtt

SEQ ID NO: 690 - DNA, 21 bp, Artificial Sequence
  Description of Artificial Sequence Synthetic oligonucleotide
  Description of Combined DNA/RNA Molecule Synthetic oligonucleotide
cagggcuguaucacaucggtt

SEQ ID NO: 691 - DNA, 21 bp, Artificial Sequence
  Description of Artificial Sequence Synthetic oligonucleotide
  Description of Combined DNA/RNA Molecule Synthetic oligonucleotide
agggcuguaucacaucggutt

SEQ ID NO: 692 - DNA, 21 bp, Artificial Sequence
  Description of Artificial Sequence Synthetic oligonucleotide
  Description of Combined DNA/RNA Molecule Synthetic oligonucleotide
ggcuguaucacaucgguuatt

SEQ ID NO: 693 - DNA, 21 bp, Artificial Sequence
  Description of Artificial Sequence Synthetic oligonucleotide
  Description of Combined DNA/RNA Molecule Synthetic oligonucleotide
gcuguaucacaucgguuautt

SEQ ID NO: 694 - DNA, 21 bp, Artificial Sequence
  Description of Artificial Sequence Synthetic oligonucleotide
  Description of Combined DNA/RNA Molecule Synthetic oligonucleotide
cuguaucacaucgguuauutt

SEQ ID NO: 695 - DNA, 21 bp, Artificial Sequence
  Description of Artificial Sequence Synthetic oligonucleotide
  Description of Combined DNA/RNA Molecule Synthetic oligonucleotide
uguaucacaucgguuauuutt

SEQ ID NO: 696 - DNA, 21 bp, Artificial Sequence
  Description of Artificial Sequence Synthetic oligonucleotide
  Description of Combined DNA/RNA Molecule Synthetic oligonucleotide
guaucacaucgguuauuuutt

SEQ ID NO: 697 - DNA, 21 bp, Artificial Sequence
  Description of Artificial Sequence Synthetic oligonucleotide
  Description of Combined DNA/RNA Molecule Synthetic oligonucleotide
uaucacaucgguuauuuugtt

SEQ ID NO: 698 - DNA, 21 bp, Artificial Sequence
  Description of Artificial Sequence Synthetic oligonucleotide
  Description of Combined DNA/RNA Molecule Synthetic oligonucleotide
ucacaucgguuauuuuggctt

SEQ ID NO: 699 - DNA, 21 bp, Artificial Sequence
  Description of Artificial Sequence Synthetic oligonucleotide
  Description of Combined DNA/RNA Molecule Synthetic oligonucleotide
acaucgguuauuuuggcggtt

SEQ ID NO: 700 - DNA, 21 bp, Artificial Sequence
  Description of Artificial Sequence Synthetic oligonucleotide
  Description of Combined DNA/RNA Molecule Synthetic oligonucleotide
caucgguuauuuuggcggatt

SEQ ID NO: 701 - DNA, 21 bp, Artificial Sequence
  Description of Artificial Sequence Synthetic oligonucleotide
  Description of Combined DNA/RNA Molecule Synthetic oligonucleotide
aucgguuauuuuggcggaatt

SEQ ID NO: 702 - DNA, 21 bp, Artificial Sequence
  Description of Artificial Sequence Synthetic oligonucleotide
  Description of Combined DNA/RNA Molecule Synthetic oligonucleotide
ucgguuauuuuggcggaautt

SEQ ID NO: 703 - DNA, 21 bp, Artificial Sequence
  Description of Artificial Sequence Synthetic oligonucleotide
  Description of Combined DNA/RNA Molecule Synthetic oligonucleotide
cgguuauuuuggcggaauctt

SEQ ID NO: 704 - DNA, 21 bp, Artificial Sequence
  Description of Artificial Sequence Synthetic oligonucleotide
  Description of Combined DNA/RNA Molecule Synthetic oligonucleotide
guuauuuuggcggaaucuutt

SEQ ID NO: 705 - DNA, 21 bp, Artificial Sequence
  Description of Artificial Sequence Synthetic oligonucleotide
  Description of Combined DNA/RNA Molecule Synthetic oligonucleotide
uuauuuuggcggaaucuuctt

SEQ ID NO: 706 - DNA, 21 bp, Artificial Sequence
  Description of Artificial Sequence Synthetic oligonucleotide
  Description of Combined DNA/RNA Molecule Synthetic oligonucleotide
uauuuuggcggaaucuucutt

SEQ ID NO: 707 - DNA, 21 bp, Artificial Sequence
  Description of Artificial Sequence Synthetic oligonucleotide
  Description of Combined DNA/RNA Molecule Synthetic oligonucleotide
auuuuggcggaaucuucuutt

SEQ ID NO: 708 - DNA, 21 bp, Artificial Sequence
  Description of Artificial Sequence Synthetic oligonucleotide
  Description of Combined DNA/RNA Molecule Synthetic oligonucleotide
uuuuggcggaaucuucuuctt

SEQ ID NO: 709 - DNA, 21 bp, Artificial Sequence
  Description of Artificial Sequence Synthetic oligonucleotide
  Description of Combined DNA/RNA Molecule Synthetic oligonucleotide
uuuggcggaaucuucuucatt

SEQ ID NO: 710 - DNA, 21 bp, Artificial Sequence
  Description of Artificial Sequence Synthetic oligonucleotide
  Description of Combined DNA/RNA Molecule Synthetic oligonucleotide
aagugugaucaccugguugtt

SEQ ID NO: 711 - DNA, 21 bp, Artificial Sequence
  Description of Artificial Sequence Synthetic oligonucleotide
  Description of Combined DNA/RNA Molecule Synthetic oligonucleotide
ugugaucaccugguuggugtt

SEQ ID NO: 712 - DNA, 21 bp, Artificial Sequence
  Description of Artificial Sequence Synthetic oligonucleotide
  Description of Combined DNA/RNA Molecule Synthetic oligonucleotide
gugaucaccugguugguggtt

SEQ ID NO: 713 - DNA, 21 bp, Artificial Sequence
  Description of Artificial Sequence Synthetic oligonucleotide
  Description of Combined DNA/RNA Molecule Synthetic oligonucleotide
ugaucaccugguugguggctt

SEQ ID NO: 714 - DNA, 21 bp, Artificial Sequence
  Description of Artificial Sequence Synthetic oligonucleotide
  Description of Combined DNA/RNA Molecule Synthetic oligonucleotide
ucaccugguugguggcugutt

SEQ ID NO: 715 - DNA, 21 bp, Artificial Sequence
  Description of Artificial Sequence Synthetic oligonucleotide
  Description of Combined DNA/RNA Molecule Synthetic oligonucleotide
caccugguugguggcugugtt

SEQ ID NO: 716 - DNA, 21 bp, Artificial Sequence
  Description of Artificial Sequence Synthetic oligonucleotide
  Description of Combined DNA/RNA Molecule Synthetic oligonucleotide
aggaaucaucuuuacuaaatt

SEQ ID NO: 717 - DNA, 21 bp, Artificial Sequence
  Description of Artificial Sequence Synthetic oligonucleotide
  Description of Combined DNA/RNA Molecule Synthetic oligonucleotide
ucaucuuuacuaaaugccatt

SEQ ID NO: 718 - DNA, 21 bp, Artificial Sequence
  Description of Artificial Sequence Synthetic oligonucleotide
  Description of Combined DNA/RNA Molecule Synthetic oligonucleotide
gaagauucuguuuaugucutt

SEQ ID NO: 719 - DNA, 21 bp, Artificial Sequence
  Description of Artificial Sequence Synthetic oligonucleotide
  Description of Combined DNA/RNA Molecule Synthetic oligonucleotide
cuguggcccuuauuuuccatt

SEQ ID NO: 720 - DNA, 21 bp, Artificial Sequence
  Description of Artificial Sequence Synthetic oligonucleotide
  Description of Combined DNA/RNA Molecule Synthetic oligonucleotide
uggcccuuauuuuccacgatt

SEQ ID NO: 721 - DNA, 21 bp, Artificial Sequence
  Description of Artificial Sequence Synthetic oligonucleotide
  Description of Combined DNA/RNA Molecule Synthetic oligonucleotide
ggcccuuauuuuccacgagtt

SEQ ID NO: 722 - DNA, 21 bp, Artificial Sequence
  Description of Artificial Sequence Synthetic oligonucleotide
  Description of Combined DNA/RNA Molecule Synthetic oligonucleotide
gcccuuauuuuccacgaggtt

SEQ ID NO: 723 - DNA, 21 bp, Artificial Sequence
  Description of Artificial Sequence Synthetic oligonucleotide
  Description of Combined DNA/RNA Molecule Synthetic oligonucleotide
cuuauuuuccacgaggaugtt

SEQ ID NO: 724 - DNA, 21 bp, Artificial Sequence
  Description of Artificial Sequence Synthetic oligonucleotide
  Description of Combined DNA/RNA Molecule Synthetic oligonucleotide
uuauuuuccacgaggauggtt

SEQ ID NO: 725 - DNA, 21 bp, Artificial Sequence
  Description of Artificial Sequence Synthetic oligonucleotide
  Description of Combined DNA/RNA Molecule Synthetic oligonucleotide
uauuuuccacgaggauggatt

SEQ ID NO: 726 - DNA, 21 bp, Artificial Sequence
  Description of Artificial Sequence Synthetic oligonucleotide
  Description of Combined DNA/RNA Molecule Synthetic oligonucleotide
uuuccacgaggauggaauatt

SEQ ID NO: 727 - DNA, 21 bp, Artificial Sequence
  Description of Artificial Sequence Synthetic oligonucleotide
  Description of Combined DNA/RNA Molecule Synthetic oligonucleotide
uuccacgaggauggaauaatt

SEQ ID NO: 728 - DNA, 21 bp, Artificial Sequence
  Description of Artificial Sequence Synthetic oligonucleotide
  Description of Combined DNA/RNA Molecule Synthetic oligonucleotide
uccacgaggauggaauaautt

SEQ ID NO: 729 - DNA, 21 bp, Artificial Sequence
  Description of Artificial Sequence Synthetic oligonucleotide
  Description of Combined DNA/RNA Molecule Synthetic oligonucleotide
cacgaggauggaauaauuutt

SEQ ID NO: 730 - DNA, 21 bp, Artificial Sequence
  Description of Artificial Sequence Synthetic oligonucleotide
  Description of Combined DNA/RNA Molecule Synthetic oligonucleotide
aauuuccacacaauaaugatt

SEQ ID NO: 731 - DNA, 21 bp, Artificial Sequence
  Description of Artificial Sequence Synthetic oligonucleotide
  Description of Combined DNA/RNA Molecule Synthetic oligonucleotide
uuuccacacaauaaugaggtt

SEQ ID NO: 732 - DNA, 21 bp, Artificial Sequence
  Description of Artificial Sequence Synthetic oligonucleotide
  Description of Combined DNA/RNA Molecule Synthetic oligonucleotide
cacacaauaaugaggaacatt

SEQ ID NO: 733 - DNA, 21 bp, Artificial Sequence
  Description of Artificial Sequence Synthetic oligonucleotide
  Description of Combined DNA/RNA Molecule Synthetic oligonucleotide
uaaugaggaacauuuugggtt

SEQ ID NO: 734 - DNA, 21 bp, Artificial Sequence
  Description of Artificial Sequence Synthetic oligonucleotide
  Description of Combined DNA/RNA Molecule Synthetic oligonucleotide
aacauuuuggggcuggucctt

SEQ ID NO: 735 - DNA, 21 bp, Artificial Sequence
  Description of Artificial Sequence Synthetic oligonucleotide
  Description of Combined DNA/RNA Molecule Synthetic oligonucleotide
gccgcugcucaucaugguctt

SEQ ID NO: 736 - DNA, 21 bp, Artificial Sequence
  Description of Artificial Sequence Synthetic oligonucleotide
  Description of Combined DNA/RNA Molecule Synthetic oligonucleotide
aauucuucggccugaguaatt

SEQ ID NO: 737 - DNA, 21 bp, Artificial Sequence
  Description of Artificial Sequence Synthetic oligonucleotide
  Description of Combined DNA/RNA Molecule Synthetic oligonucleotide
auucuucggccugaguaactt

SEQ ID NO: 738 - DNA, 21 bp, Artificial Sequence
  Description of Artificial Sequence Synthetic oligonucleotide
  Description of Combined DNA/RNA Molecule Synthetic oligonucleotide
uucuucggccugaguaacutt

SEQ ID NO: 739 - DNA, 21 bp, Artificial Sequence
  Description of Artificial Sequence Synthetic oligonucleotide
  Description of Combined DNA/RNA Molecule Synthetic oligonucleotide
uucggccugaguaacugugtt

SEQ ID NO: 740 - DNA, 21 bp, Artificial Sequence
  Description of Artificial Sequence Synthetic oligonucleotide
  Description of Combined DNA/RNA Molecule Synthetic oligonucleotide
ugaguaacugugaaagcactt

SEQ ID NO: 741 - DNA, 21 bp, Artificial Sequence
  Description of Artificial Sequence Synthetic oligonucleotide
  Description of Combined DNA/RNA Molecule Synthetic oligonucleotide
caccagucaacuggaccaatt

SEQ ID NO: 742 - DNA, 21 bp, Artificial Sequence
  Description of Artificial Sequence Synthetic oligonucleotide
  Description of Combined DNA/RNA Molecule Synthetic oligonucleotide
caacuggaccaagccacgctt

SEQ ID NO: 743 - DNA, 21 bp, Artificial Sequence
  Description of Artificial Sequence Synthetic oligonucleotide
  Description of Combined DNA/RNA Molecule Synthetic oligonucleotide
aacuggaccaagccacgcatt

SEQ ID NO: 744 - DNA, 21 bp, Artificial Sequence
  Description of Artificial Sequence Synthetic oligonucleotide
  Description of Combined DNA/RNA Molecule Synthetic oligonucleotide
cuggggaaaugcguccuugtt

SEQ ID NO: 745 - DNA, 21 bp, Artificial Sequence
  Description of Artificial Sequence Synthetic oligonucleotide
  Description of Combined DNA/RNA Molecule Synthetic oligonucleotide
acuggggaaaugcguccuutt

SEQ ID NO: 746 - DNA, 21 bp, Artificial Sequence
  Description of Artificial Sequence Synthetic oligonucleotide
  Description of Combined DNA/RNA Molecule Synthetic oligonucleotide
guacuggggaaaugcgucctt

SEQ ID NO: 747 - DNA, 21 bp, Artificial Sequence
  Description of Artificial Sequence Synthetic oligonucleotide
  Description of Combined DNA/RNA Molecule Synthetic oligonucleotide
uguacuggggaaaugcguctt

SEQ ID NO: 748 - DNA, 21 bp, Artificial Sequence
  Description of Artificial Sequence Synthetic oligonucleotide
  Description of Combined DNA/RNA Molecule Synthetic oligonucleotide
auguacuggggaaaugcgutt

SEQ ID NO: 749 - DNA, 21 bp, Artificial Sequence
  Description of Artificial Sequence Synthetic oligonucleotide
  Description of Combined DNA/RNA Molecule Synthetic oligonucleotide
gauguacuggggaaaugcgtt

SEQ ID NO: 750 - DNA, 21 bp, Artificial Sequence
  Description of Artificial Sequence Synthetic oligonucleotide
  Description of Combined DNA/RNA Molecule Synthetic oligonucleotide
uggauguacuggggaaaugtt

SEQ ID NO: 751 - DNA, 21 bp, Artificial Sequence
  Description of Artificial Sequence Synthetic oligonucleotide
  Description of Combined DNA/RNA Molecule Synthetic oligonucleotide
guggauguacuggggaaautt

SEQ ID NO: 752 - DNA, 21 bp, Artificial Sequence
  Description of Artificial Sequence Synthetic oligonucleotide
  Description of Combined DNA/RNA Molecule Synthetic oligonucleotide
uguggauguacuggggaaatt

SEQ ID NO: 753 - DNA, 21 bp, Artificial Sequence
  Description of Artificial Sequence Synthetic oligonucleotide
  Description of Combined DNA/RNA Molecule Synthetic oligonucleotide
guuguggauguacuggggatt

SEQ ID NO: 754 - DNA, 21 bp, Artificial Sequence
  Description of Artificial Sequence Synthetic oligonucleotide
  Description of Combined DNA/RNA Molecule Synthetic oligonucleotide
gcauguuguggauguacugtt

SEQ ID NO: 755 - DNA, 21 bp, Artificial Sequence
  Description of Artificial Sequence Synthetic oligonucleotide
  Description of Combined DNA/RNA Molecule Synthetic oligonucleotide
cagcauguuguggauguactt

SEQ ID NO: 756 - DNA, 21 bp, Artificial Sequence
  Description of Artificial Sequence Synthetic oligonucleotide
  Description of Combined DNA/RNA Molecule Synthetic oligonucleotide
gacagcauguuguggaugutt

SEQ ID NO: 757 - DNA, 21 bp, Artificial Sequence
  Description of Artificial Sequence Synthetic oligonucleotide
  Description of Combined DNA/RNA Molecule Synthetic oligonucleotide
aacgagauguggacagcautt

SEQ ID NO: 758 - DNA, 21 bp, Artificial Sequence
  Description of Artificial Sequence Synthetic oligonucleotide
  Description of Combined DNA/RNA Molecule Synthetic oligonucleotide
gaacgagauguggacagcatt

SEQ ID NO: 759 - DNA, 21 bp, Artificial Sequence
  Description of Artificial Sequence Synthetic oligonucleotide
  Description of Combined DNA/RNA Molecule Synthetic oligonucleotide
agaacgagauguggacagctt

SEQ ID NO: 760 - DNA, 21 bp, Artificial Sequence
  Description of Artificial Sequence Synthetic oligonucleotide
  Description of Combined DNA/RNA Molecule Synthetic oligonucleotide
gagaacgagauguggacagtt

SEQ ID NO: 761 - DNA, 21 bp, Artificial Sequence
  Description of Artificial Sequence Synthetic oligonucleotide
  Description of Combined DNA/RNA Molecule Synthetic oligonucleotide
ccgagaacgagauguggactt

SEQ ID NO: 762 - DNA, 21 bp, Artificial Sequence
  Description of Artificial Sequence Synthetic oligonucleotide
  Description of Combined DNA/RNA Molecule Synthetic oligonucleotide
accgagaacgagauguggatt

SEQ ID NO: 763 - DNA, 21 bp, Artificial Sequence
  Description of Artificial Sequence Synthetic oligonucleotide
  Description of Combined DNA/RNA Molecule Synthetic oligonucleotide
aaccgagaacgagauguggtt

SEQ ID NO: 764 - DNA, 21 bp, Artificial Sequence
  Description of Artificial Sequence Synthetic oligonucleotide
  Description of Combined DNA/RNA Molecule Synthetic oligonucleotide
aaaccgagaacgagaugugtt

SEQ ID NO: 765 - DNA, 21 bp, Artificial Sequence
  Description of Artificial Sequence Synthetic oligonucleotide
  Description of Combined DNA/RNA Molecule Synthetic oligonucleotide
uaaaccgagaacgagaugutt

SEQ ID NO: 766 - DNA, 21 bp, Artificial Sequence
  Description of Artificial Sequence Synthetic oligonucleotide
  Description of Combined DNA/RNA Molecule Synthetic oligonucleotide
auaaaccgagaacgagaugtt

SEQ ID NO: 767 - DNA, 21 bp, Artificial Sequence
  Description of Artificial Sequence Synthetic oligonucleotide
  Description of Combined DNA/RNA Molecule Synthetic oligonucleotide
ugauaaaccgagaacgagatt

SEQ ID NO: 768 - DNA, 21 bp, Artificial Sequence
  Description of Artificial Sequence Synthetic oligonucleotide
  Description of Combined DNA/RNA Molecule Synthetic oligonucleotide
cugauaaaccgagaacgagtt

SEQ ID NO: 769 - DNA, 21 bp, Artificial Sequence
  Description of Artificial Sequence Synthetic oligonucleotide
  Description of Combined DNA/RNA Molecule Synthetic oligonucleotide
ucugauaaaccgagaacgatt

SEQ ID NO: 770 - DNA, 21 bp, Artificial Sequence
  Description of Artificial Sequence Synthetic oligonucleotide
  Description of Combined DNA/RNA Molecule Synthetic oligonucleotide
uucugauaaaccgagaacgtt

SEQ ID NO: 771 - DNA, 21 bp, Artificial Sequence
  Description of Artificial Sequence Synthetic oligonucleotide
  Description of Combined DNA/RNA Molecule Synthetic oligonucleotide
auuucugauaaaccgagaatt

SEQ ID NO: 772 - DNA, 21 bp, Artificial Sequence
  Description of Artificial Sequence Synthetic oligonucleotide
  Description of Combined DNA/RNA Molecule Synthetic oligonucleotide
uauuucugauaaaccgagatt

SEQ ID NO: 773 - DNA, 21 bp, Artificial Sequence
  Description of Artificial Sequence Synthetic oligonucleotide
  Description of Combined DNA/RNA Molecule Synthetic oligonucleotide
guauuucugauaaaccgagtt

SEQ ID NO: 774 - DNA, 21 bp, Artificial Sequence
  Description of Artificial Sequence Synthetic oligonucleotide
  Description of Combined DNA/RNA Molecule Synthetic oligonucleotide
gguauuucugauaaaccgatt

SEQ ID NO: 775 - DNA, 21 bp, Artificial Sequence
  Description of Artificial Sequence Synthetic oligonucleotide
  Description of Combined DNA/RNA Molecule Synthetic oligonucleotide
cguugguauuucugauaaatt

SEQ ID NO: 776 - DNA, 21 bp, Artificial Sequence
  Description of Artificial Sequence Synthetic oligonucleotide
  Description of Combined DNA/RNA Molecule Synthetic oligonucleotide
gugacuucuucaccgcucutt

SEQ ID NO: 777 - DNA, 21 bp, Artificial Sequence
  Description of Artificial Sequence Synthetic oligonucleotide
  Description of Combined DNA/RNA Molecule Synthetic oligonucleotide
gguggugacuucuucaccgtt

SEQ ID NO: 778 - DNA, 21 bp, Artificial Sequence
  Description of Artificial Sequence Synthetic oligonucleotide
  Description of Combined DNA/RNA Molecule Synthetic oligonucleotide
ucaaaaaagguggugacuutt

SEQ ID NO: 779 - DNA, 21 bp, Artificial Sequence
  Description of Artificial Sequence Synthetic oligonucleotide
  Description of Combined DNA/RNA Molecule Synthetic oligonucleotide
uaaucaaaaaagguggugatt

SEQ ID NO: 780 - DNA, 21 bp, Artificial Sequence
  Description of Artificial Sequence Synthetic oligonucleotide
  Description of Combined DNA/RNA Molecule Synthetic oligonucleotide
auaaucaaaaaagguggugtt

SEQ ID NO: 781 - DNA, 21 bp, Artificial Sequence
  Description of Artificial Sequence Synthetic oligonucleotide
  Description of Combined DNA/RNA Molecule Synthetic oligonucleotide
cauaaucaaaaaagguggutt

SEQ ID NO: 782 - DNA, 21 bp, Artificial Sequence
  Description of Artificial Sequence Synthetic oligonucleotide
  Description of Combined DNA/RNA Molecule Synthetic oligonucleotide
accguaaucauaaucaaaatt

SEQ ID NO: 783 - DNA, 21 bp, Artificial Sequence
  Description of Artificial Sequence Synthetic oligonucleotide
  Description of Combined DNA/RNA Molecule Synthetic oligonucleotide
agcaccguaaucauaaucatt

SEQ ID NO: 784 - DNA, 21 bp, Artificial Sequence
  Description of Artificial Sequence Synthetic oligonucleotide
  Description of Combined DNA/RNA Molecule Synthetic oligonucleotide
gggagcaccguaaucauaatt

SEQ ID NO: 785 - DNA, 21 bp, Artificial Sequence
  Description of Artificial Sequence Synthetic oligonucleotide
  Description of Combined DNA/RNA Molecule Synthetic oligonucleotide
agggagcaccguaaucauatt

SEQ ID NO: 786 - DNA, 21 bp, Artificial Sequence
  Description of Artificial Sequence Synthetic oligonucleotide
  Description of Combined DNA/RNA Molecule Synthetic oligonucleotide
acagggagcaccguaaucatt

SEQ ID NO: 787 - DNA, 21 bp, Artificial Sequence
  Description of Artificial Sequence Synthetic oligonucleotide
  Description of Combined DNA/RNA Molecule Synthetic oligonucleotide
gacagggagcaccguaauctt

SEQ ID NO: 788 - DNA, 21 bp, Artificial Sequence
  Description of Artificial Sequence Synthetic oligonucleotide
  Description of Combined DNA/RNA Molecule Synthetic oligonucleotide
ugacagggagcaccguaautt

SEQ ID NO: 789 - DNA, 21 bp, Artificial Sequence
  Description of Artificial Sequence Synthetic oligonucleotide
  Description of Combined DNA/RNA Molecule Synthetic oligonucleotide
augacagggagcaccguaatt

SEQ ID NO: 790 - DNA, 21 bp, Artificial Sequence
  Description of Artificial Sequence Synthetic oligonucleotide
  Description of Combined DNA/RNA Molecule Synthetic oligonucleotide
uuaugacagggagcaccgutt

SEQ ID NO: 791 - DNA, 21 bp, Artificial Sequence
  Description of Artificial Sequence Synthetic oligonucleotide
  Description of Combined DNA/RNA Molecule Synthetic oligonucleotide
aauuuaugacagggagcactt

SEQ ID NO: 792 - DNA, 21 bp, Artificial Sequence
  Description of Artificial Sequence Synthetic oligonucleotide
  Description of Combined DNA/RNA Molecule Synthetic oligonucleotide
aaauuuaugacagggagcatt

SEQ ID NO: 793 - DNA, 21 bp, Artificial Sequence
  Description of Artificial Sequence Synthetic oligonucleotide
  Description of Combined DNA/RNA Molecule Synthetic oligonucleotide
ucaaauuuaugacagggagtt

SEQ ID NO: 794 - DNA, 21 bp, Artificial Sequence
  Description of Artificial Sequence Synthetic oligonucleotide
  Description of Combined DNA/RNA Molecule Synthetic oligonucleotide
acgucaaauuuaugacaggtt

SEQ ID NO: 795 - DNA, 21 bp, Artificial Sequence
  Description of Artificial Sequence Synthetic oligonucleotide
  Description of Combined DNA/RNA Molecule Synthetic oligonucleotide
ucacgucaaauuuaugacatt

SEQ ID NO: 796 - DNA, 21 bp, Artificial Sequence
  Description of Artificial Sequence Synthetic oligonucleotide
  Description of Combined DNA/RNA Molecule Synthetic oligonucleotide
uucacgucaaauuuaugactt

SEQ ID NO: 797 - DNA, 21 bp, Artificial Sequence
  Description of Artificial Sequence Synthetic oligonucleotide
  Description of Combined DNA/RNA Molecule Synthetic oligonucleotide
cuucacgucaaauuuaugatt

SEQ ID NO: 798 - DNA, 21 bp, Artificial Sequence
  Description of Artificial Sequence Synthetic oligonucleotide
  Description of Combined DNA/RNA Molecule Synthetic oligonucleotide
ugcuucacgucaaauuuautt

SEQ ID NO: 799 - DNA, 21 bp, Artificial Sequence
  Description of Artificial Sequence Synthetic oligonucleotide
  Description of Combined DNA/RNA Molecule Synthetic oligonucleotide
auuugcuucacgucaaauutt

SEQ ID NO: 800 - DNA, 21 bp, Artificial Sequence
  Description of Artificial Sequence Synthetic oligonucleotide
  Description of Combined DNA/RNA Molecule Synthetic oligonucleotide
aauuugcuucacgucaaautt

SEQ ID NO: 801 - DNA, 21 bp, Artificial Sequence
  Description of Artificial Sequence Synthetic oligonucleotide
  Description of Combined DNA/RNA Molecule Synthetic oligonucleotide
caauuugcuucacgucaaatt

SEQ ID NO: 802 - DNA, 21 bp, Artificial Sequence
  Description of Artificial Sequence Synthetic oligonucleotide
  Description of Combined DNA/RNA Molecule Synthetic oligonucleotide
ccaauuugcuucacgucaatt

SEQ ID NO: 803 - DNA, 21 bp, Artificial Sequence
  Description of Artificial Sequence Synthetic oligonucleotide
  Description of Combined DNA/RNA Molecule Synthetic oligonucleotide
cccaauuugcuucacgucatt

SEQ ID NO: 804 - DNA, 21 bp, Artificial Sequence
  Description of Artificial Sequence Synthetic oligonucleotide
  Description of Combined DNA/RNA Molecule Synthetic oligonucleotide
ccccaauuugcuucacguctt

SEQ ID NO: 805 - DNA, 21 bp, Artificial Sequence
  Description of Artificial Sequence Synthetic oligonucleotide
  Description of Combined DNA/RNA Molecule Synthetic oligonucleotide
gccccaauuugcuucacgutt

SEQ ID NO: 806 - DNA, 21 bp, Artificial Sequence
  Description of Artificial Sequence Synthetic oligonucleotide
  Description of Combined DNA/RNA Molecule Synthetic oligonucleotide
ggccccaauuugcuucacgtt

SEQ ID NO: 807 - DNA, 21 bp, Artificial Sequence
  Description of Artificial Sequence Synthetic oligonucleotide
  Description of Combined DNA/RNA Molecule Synthetic oligonucleotide
uugggccccaauuugcuuctt

SEQ ID NO: 808 - DNA, 21 bp, Artificial Sequence
  Description of Artificial Sequence Synthetic oligonucleotide
  Description of Combined DNA/RNA Molecule Synthetic oligonucleotide
guugggccccaauuugcuutt

SEQ ID NO: 809 - DNA, 21 bp, Artificial Sequence
  Description of Artificial Sequence Synthetic oligonucleotide
  Description of Combined DNA/RNA Molecule Synthetic oligonucleotide
aguugggccccaauuugcutt

SEQ ID NO: 810 - DNA, 21 bp, Artificial Sequence
  Description of Artificial Sequence Synthetic oligonucleotide
  Description of Combined DNA/RNA Molecule Synthetic oligonucleotide
gaguugggccccaauuugctt

SEQ ID NO: 811 - DNA, 21 bp, Artificial Sequence
  Description of Artificial Sequence Synthetic oligonucleotide
  Description of Combined DNA/RNA Molecule Synthetic oligonucleotide
agcacuucagcuuuuugcatt

SEQ ID NO: 812 - DNA, 21 bp, Artificial Sequence
  Description of Artificial Sequence Synthetic oligonucleotide
  Description of Combined DNA/RNA Molecule Synthetic oligonucleotide
agucaagcacuucagcuuutt

SEQ ID NO: 813 - DNA, 21 bp, Artificial Sequence
  Description of Artificial Sequence Synthetic oligonucleotide
  Description of Combined DNA/RNA Molecule Synthetic oligonucleotide
gguaaaugucagucaagcatt

SEQ ID NO: 814 - DNA, 21 bp, Artificial Sequence
  Description of Artificial Sequence Synthetic oligonucleotide
  Description of Combined DNA/RNA Molecule Synthetic oligonucleotide
cagguaaaugucagucaagtt

SEQ ID NO: 815 - DNA, 21 bp, Artificial Sequence
  Description of Artificial Sequence Synthetic oligonucleotide
  Description of Combined DNA/RNA Molecule Synthetic oligonucleotide
agaguaauaagaaaaagcatt

SEQ ID NO: 816 - DNA, 21 bp, Artificial Sequence
  Description of Artificial Sequence Synthetic oligonucleotide
  Description of Combined DNA/RNA Molecule Synthetic oligonucleotide
aaugggagaguaauaagaatt

SEQ ID NO: 817 - DNA, 21 bp, Artificial Sequence
  Description of Artificial Sequence Synthetic oligonucleotide
  Description of Combined DNA/RNA Molecule Synthetic oligonucleotide
agugagcccacaaugggagtt

SEQ ID NO: 818 - DNA, 21 bp, Artificial Sequence
  Description of Artificial Sequence Synthetic oligonucleotide
  Description of Combined DNA/RNA Molecule Synthetic oligonucleotide
agagugagcccacaaugggtt

SEQ ID NO: 819 - DNA, 21 bp, Artificial Sequence
  Description of Artificial Sequence Synthetic oligonucleotide
  Description of Combined DNA/RNA Molecule Synthetic oligonucleotide
acucauuugcagcagagugtt

SEQ ID NO: 820 - DNA, 21 bp, Artificial Sequence
  Description of Artificial Sequence Synthetic oligonucleotide
  Description of Combined DNA/RNA Molecule Synthetic oligonucleotide
cacucauuugcagcagagutt

SEQ ID NO: 821 - DNA, 21 bp, Artificial Sequence
  Description of Artificial Sequence Synthetic oligonucleotide
  Description of Combined DNA/RNA Molecule Synthetic oligonucleotide
gcauucccaaagacccacutt

SEQ ID NO: 822 - DNA, 21 bp, Artificial Sequence
  Description of Artificial Sequence Synthetic oligonucleotide
  Description of Combined DNA/RNA Molecule Synthetic oligonucleotide
ugcauucccaaagacccactt

SEQ ID NO: 823 - DNA, 21 bp, Artificial Sequence
  Description of Artificial Sequence Synthetic oligonucleotide
  Description of Combined DNA/RNA Molecule Synthetic oligonucleotide
auugcauucccaaagaccctt

SEQ ID NO: 824 - DNA, 21 bp, Artificial Sequence
  Description of Artificial Sequence Synthetic oligonucleotide
  Description of Combined DNA/RNA Molecule Synthetic oligonucleotide
uugcacauugcauucccaatt

SEQ ID NO: 825 - DNA, 21 bp, Artificial Sequence
  Description of Artificial Sequence Synthetic oligonucleotide
  Description of Combined DNA/RNA Molecule Synthetic oligonucleotide
cccugugaauaauuugcactt

SEQ ID NO: 826 - DNA, 21 bp, Artificial Sequence
  Description of Artificial Sequence Synthetic oligonucleotide
  Description of Combined DNA/RNA Molecule Synthetic oligonucleotide
gaugugauacagcccugugtt

SEQ ID NO: 827 - DNA, 21 bp, Artificial Sequence
  Description of Artificial Sequence Synthetic oligonucleotide
  Description of Combined DNA/RNA Molecule Synthetic oligonucleotide
cgaugugauacagcccugutt

SEQ ID NO: 828 - DNA, 21 bp, Artificial Sequence
  Description of Artificial Sequence Synthetic oligonucleotide
  Description of Combined DNA/RNA Molecule Synthetic oligonucleotide
ccgaugugauacagcccugtt

SEQ ID NO: 829 - DNA, 21 bp, Artificial Sequence
  Description of Artificial Sequence Synthetic oligonucleotide
  Description of Combined DNA/RNA Molecule Synthetic oligonucleotide
accgaugugauacagcccutt

SEQ ID NO: 830 - DNA, 21 bp, Artificial Sequence
  Description of Artificial Sequence Synthetic oligonucleotide
  Description of Combined DNA/RNA Molecule Synthetic oligonucleotide
uaaccgaugugauacagcctt

SEQ ID NO: 831 - DNA, 21 bp, Artificial Sequence
  Description of Artificial Sequence Synthetic oligonucleotide
  Description of Combined DNA/RNA Molecule Synthetic oligonucleotide
auaaccgaugugauacagctt

SEQ ID NO: 832 - DNA, 21 bp, Artificial Sequence
  Description of Artificial Sequence Synthetic oligonucleotide
  Description of Combined DNA/RNA Molecule Synthetic oligonucleotide
aauaaccgaugugauacagtt

SEQ ID NO: 833 - DNA, 21 bp, Artificial Sequence
  Description of Artificial Sequence Synthetic oligonucleotide
  Description of Combined DNA/RNA Molecule Synthetic oligonucleotide
aaauaaccgaugugauacatt

SEQ ID NO: 834 - DNA, 21 bp, Artificial Sequence
  Description of Artificial Sequence Synthetic oligonucleotide
  Description of Combined DNA/RNA Molecule Synthetic oligonucleotide
aaaauaaccgaugugauactt

SEQ ID NO: 835 - DNA, 21 bp, Artificial Sequence
  Description of Artificial Sequence Synthetic oligonucleotide
  Description of Combined DNA/RNA Molecule Synthetic oligonucleotide
caaaauaaccgaugugauatt

SEQ ID NO: 836 - DNA, 21 bp, Artificial Sequence
  Description of Artificial Sequence Synthetic oligonucleotide
  Description of Combined DNA/RNA Molecule Synthetic oligonucleotide
gccaaaauaaccgaugugatt

SEQ ID NO: 837 - DNA, 21 bp, Artificial Sequence
  Description of Artificial Sequence Synthetic oligonucleotide
  Description of Combined DNA/RNA Molecule Synthetic oligonucleotide
ccgccaaaauaaccgaugutt

SEQ ID NO: 838 - DNA, 21 bp, Artificial Sequence
  Description of Artificial Sequence Synthetic oligonucleotide
  Description of Combined DNA/RNA Molecule Synthetic oligonucleotide
uccgccaaaauaaccgaugtt

SEQ ID NO: 839 - DNA, 21 bp, Artificial Sequence
  Description of Artificial Sequence Synthetic oligonucleotide
  Description of Combined DNA/RNA Molecule Synthetic oligonucleotide
uuccgccaaaauaaccgautt

SEQ ID NO: 840 - DNA, 21 bp, Artificial Sequence
  Description of Artificial Sequence Synthetic oligonucleotide
  Description of Combined DNA/RNA Molecule Synthetic oligonucleotide
auuccgccaaaauaaccgatt

SEQ ID NO: 841 - DNA, 21 bp, Artificial Sequence
  Description of Artificial Sequence Synthetic oligonucleotide
  Description of Combined DNA/RNA Molecule Synthetic oligonucleotide
gauuccgccaaaauaaccgtt

SEQ ID NO: 842 - DNA, 21 bp, Artificial Sequence
  Description of Artificial Sequence Synthetic oligonucleotide
  Description of Combined DNA/RNA Molecule Synthetic oligonucleotide
aagauuccgccaaaauaactt

SEQ ID NO: 843 - DNA, 21 bp, Artificial Sequence
  Description of Artificial Sequence Synthetic oligonucleotide
  Description of Combined DNA/RNA Molecule Synthetic oligonucleotide
gaagauuccgccaaaauaatt

SEQ ID NO: 844 - DNA, 21 bp, Artificial Sequence
  Description of Artificial Sequence Synthetic oligonucleotide
  Description of Combined DNA/RNA Molecule Synthetic oligonucleotide
agaagauuccgccaaaauatt

SEQ ID NO: 845 - DNA, 21 bp, Artificial Sequence
  Description of Artificial Sequence Synthetic oligonucleotide
  Description of Combined DNA/RNA Molecule Synthetic oligonucleotide
aagaagauuccgccaaaautt

SEQ ID NO: 846 - DNA, 21 bp, Artificial Sequence
  Description of Artificial Sequence Synthetic oligonucleotide
  Description of Combined DNA/RNA Molecule Synthetic oligonucleotide
gaagaagauuccgccaaaatt

SEQ ID NO: 847 - DNA, 21 bp, Artificial Sequence
  Description of Artificial Sequence Synthetic oligonucleotide
  Description of Combined DNA/RNA Molecule Synthetic oligonucleotide
ugaagaagauuccgccaaatt

SEQ ID NO: 848 - DNA, 21 bp, Artificial Sequence
  Description of Artificial Sequence Synthetic oligonucleotide
  Description of Combined DNA/RNA Molecule Synthetic oligonucleotide
caaccaggugaucacacuutt

SEQ ID NO: 849 - DNA, 21 bp, Artificial Sequence
  Description of Artificial Sequence Synthetic oligonucleotide
  Description of Combined DNA/RNA Molecule Synthetic oligonucleotide
caccaaccaggugaucacatt

SEQ ID NO: 850 - DNA, 21 bp, Artificial Sequence
  Description of Artificial Sequence Synthetic oligonucleotide
  Description of Combined DNA/RNA Molecule Synthetic oligonucleotide
ccaccaaccaggugaucactt

SEQ ID NO: 851 - DNA, 21 bp, Artificial Sequence
  Description of Artificial Sequence Synthetic oligonucleotide
  Description of Combined DNA/RNA Molecule Synthetic oligonucleotide
gccaccaaccaggugaucatt

SEQ ID NO: 852 - DNA, 21 bp, Artificial Sequence
  Description of Artificial Sequence Synthetic oligonucleotide
  Description of Combined DNA/RNA Molecule Synthetic oligonucleotide
acagccaccaaccaggugatt

SEQ ID NO: 853 - DNA, 21 bp, Artificial Sequence
  Description of Artificial Sequence Synthetic oligonucleotide
  Description of Combined DNA/RNA Molecule Synthetic oligonucleotide
cacagccaccaaccaggugtt

SEQ ID NO: 854 - DNA, 21 bp, Artificial Sequence
  Description of Artificial Sequence Synthetic oligonucleotide
  Description of Combined DNA/RNA Molecule Synthetic oligonucleotide
uuuaguaaagaugauuccutt

SEQ ID NO: 855 - DNA, 21 bp, Artificial Sequence
  Description of Artificial Sequence Synthetic oligonucleotide
  Description of Combined DNA/RNA Molecule Synthetic oligonucleotide
uggcauuuaguaaagaugatt

SEQ ID NO: 856 - DNA, 21 bp, Artificial Sequence
  Description of Artificial Sequence Synthetic oligonucleotide
  Description of Combined DNA/RNA Molecule Synthetic oligonucleotide
agacauaaacagaaucuuctt

SEQ ID NO: 857 - DNA, 21 bp, Artificial Sequence
  Description of Artificial Sequence Synthetic oligonucleotide
  Description of Combined DNA/RNA Molecule Synthetic oligonucleotide
uggaaaauaagggccacagtt

SEQ ID NO: 858 - DNA, 21 bp, Artificial Sequence
  Description of Artificial Sequence Synthetic oligonucleotide
  Description of Combined DNA/RNA Molecule Synthetic oligonucleotide
ucguggaaaauaagggccatt

SEQ ID NO: 859 - DNA, 21 bp, Artificial Sequence
  Description of Artificial Sequence Synthetic oligonucleotide
  Description of Combined DNA/RNA Molecule Synthetic oligonucleotide
cucguggaaaauaagggcctt

SEQ ID NO: 860 - DNA, 21 bp, Artificial Sequence
  Description of Artificial Sequence Synthetic oligonucleotide
  Description of Combined DNA/RNA Molecule Synthetic oligonucleotide
ccucguggaaaauaagggctt

SEQ ID NO: 861 - DNA, 21 bp, Artificial Sequence
  Description of Artificial Sequence Synthetic oligonucleotide
  Description of Combined DNA/RNA Molecule Synthetic oligonucleotide
cauccucguggaaaauaagtt

SEQ ID NO: 862 - DNA, 21 bp, Artificial Sequence
  Description of Artificial Sequence Synthetic oligonucleotide
  Description of Combined DNA/RNA Molecule Synthetic oligonucleotide
ccauccucguggaaaauaatt

SEQ ID NO: 863 - DNA, 21 bp, Artificial Sequence
  Description of Artificial Sequence Synthetic oligonucleotide
  Description of Combined DNA/RNA Molecule Synthetic oligonucleotide
uccauccucguggaaaauatt

SEQ ID NO: 864 - DNA, 21 bp, Artificial Sequence
  Description of Artificial Sequence Synthetic oligonucleotide
  Description of Combined DNA/RNA Molecule Synthetic oligonucleotide
uauuccauccucguggaaatt

SEQ ID NO: 865 - DNA, 21 bp, Artificial Sequence
  Description of Artificial Sequence Synthetic oligonucleotide
  Description of Combined DNA/RNA Molecule Synthetic oligonucleotide
uuauuccauccucguggaatt

SEQ ID NO: 866 - DNA, 21 bp, Artificial Sequence
  Description of Artificial Sequence Synthetic oligonucleotide
  Description of Combined DNA/RNA Molecule Synthetic oligonucleotide
auuauuccauccucguggatt

SEQ ID NO: 867 - DNA, 21 bp, Artificial Sequence
  Description of Artificial Sequence Synthetic oligonucleotide
  Description of Combined DNA/RNA Molecule Synthetic oligonucleotide
aaauuauuccauccucgugtt

SEQ ID NO: 868 - DNA, 21 bp, Artificial Sequence
  Description of Artificial Sequence Synthetic oligonucleotide
  Description of Combined DNA/RNA Molecule Synthetic oligonucleotide
ucauuauuguguggaaauutt

SEQ ID NO: 869 - DNA, 21 bp, Artificial Sequence
  Description of Artificial Sequence Synthetic oligonucleotide
  Description of Combined DNA/RNA Molecule Synthetic oligonucleotide
ccucauuauuguguggaaatt

SEQ ID NO: 870 - DNA, 21 bp, Artificial Sequence
  Description of Artificial Sequence Synthetic oligonucleotide
  Description of Combined DNA/RNA Molecule Synthetic oligonucleotide
uguuccucauuauugugugtt

SEQ ID NO: 871 - DNA, 21 bp, Artificial Sequence
  Description of Artificial Sequence Synthetic oligonucleotide
  Description of Combined DNA/RNA Molecule Synthetic oligonucleotide
cccaaaauguuccucauuatt

SEQ ID NO: 872 - DNA, 21 bp, Artificial Sequence
  Description of Artificial Sequence Synthetic oligonucleotide
  Description of Combined DNA/RNA Molecule Synthetic oligonucleotide
ggaccagccccaaaauguutt

SEQ ID NO: 873 - DNA, 21 bp, Artificial Sequence
  Description of Artificial Sequence Synthetic oligonucleotide
  Description of Combined DNA/RNA Molecule Synthetic oligonucleotide
gaccaugaugagcagcggctt

SEQ ID NO: 874 - DNA, 21 bp, Artificial Sequence
  Description of Artificial Sequence Synthetic oligonucleotide
  Description of Combined DNA/RNA Molecule Synthetic oligonucleotide
uuacucaggccgaagaauutt

SEQ ID NO: 875 - DNA, 21 bp, Artificial Sequence
  Description of Artificial Sequence Synthetic oligonucleotide
  Description of Combined DNA/RNA Molecule Synthetic oligonucleotide
guuacucaggccgaagaautt

SEQ ID NO: 876 - DNA, 21 bp, Artificial Sequence
  Description of Artificial Sequence Synthetic oligonucleotide
  Description of Combined DNA/RNA Molecule Synthetic oligonucleotide
aguuacucaggccgaagaatt

SEQ ID NO: 877 - DNA, 21 bp, Artificial Sequence
  Description of Artificial Sequence Synthetic oligonucleotide
  Description of Combined DNA/RNA Molecule Synthetic oligonucleotide
cacaguuacucaggccgaatt

SEQ ID NO: 878 - DNA, 21 bp, Artificial Sequence
  Description of Artificial Sequence Synthetic oligonucleotide
  Description of Combined DNA/RNA Molecule Synthetic oligonucleotide
gugcuuucacaguuacucatt

SEQ ID NO: 879 - DNA, 21 bp, Artificial Sequence
  Description of Artificial Sequence Synthetic oligonucleotide
  Description of Combined DNA/RNA Molecule Synthetic oligonucleotide
uugguccaguugacuggugtt

SEQ ID NO: 880 - DNA, 21 bp, Artificial Sequence
  Description of Artificial Sequence Synthetic oligonucleotide
  Description of Combined DNA/RNA Molecule Synthetic oligonucleotide
gcguggcuugguccaguugtt

SEQ ID NO: 881 - DNA, 21 bp, Artificial Sequence
  Description of Artificial Sequence Synthetic oligonucleotide
  Description of Combined DNA/RNA Molecule Synthetic oligonucleotide
ugcguggcuugguccaguutt

SEQ ID NO: 882 - protein, 7 aa, Artificial Sequence
  Description of Artificial Sequence Synthetic peptide
LGTFLKC

SEQ ID NO: 883 - RNA, 19 nt, Artificial Sequence
  Description of Artificial Sequence Synthetic oligonucleotide
uccgcuggaagaucaucga

SEQ ID NO: 884 - RNA, 19 nt, Artificial Sequence
  Description of Artificial Sequence Synthetic oligonucleotide
caucgagagcuaugaggga

SEQ ID NO: 885 - RNA, 19 nt, Artificial Sequence
  Description of Artificial Sequence Synthetic oligonucleotide
ugaacagcaaguucuauaa

SEQ ID NO: 886 - RNA, 19 nt, Artificial Sequence
  Description of Artificial Sequence Synthetic oligonucleotide
agagcgggacuauaccaaa

SEQ ID NO: 887 - RNA, 19 nt, Artificial Sequence
  Description of Artificial Sequence Synthetic oligonucleotide
ugacucauccuaacuaaca

SEQ ID NO: 888 - RNA, 19 nt, Artificial Sequence
  Description of Artificial Sequence Synthetic oligonucleotide
uccgcuggaagaucaucga

SEQ ID NO: 889 - RNA, 19 nt, Artificial Sequence
  Description of Artificial Sequence Synthetic oligonucleotide
caucgagagcuaugaggga

SEQ ID NO: 890 - RNA, 19 nt, Artificial Sequence
  Description of Artificial Sequence Synthetic oligonucleotide
ugaacagcaaguucuauaa

SEQ ID NO: 891 - RNA, 19 nt, Artificial Sequence
  Description of Artificial Sequence Synthetic oligonucleotide
agagcgggacuauaccaaa

SEQ ID NO: 892 - RNA, 19 nt, Artificial Sequence
  Description of Artificial Sequence Synthetic oligonucleotide
ugacucauccuaacuaaca

SEQ ID NO: 893 - RNA, 19 nt, Artificial Sequence
  Description of Artificial Sequence Synthetic oligonucleotide
uccgcuggaagaucaucga

SEQ ID NO: 894 - RNA, 19 nt, Artificial Sequence
  Description of Artificial Sequence Synthetic oligonucleotide
caucgagagcuaugaggga

SEQ ID NO: 895 - RNA, 19 nt, Artificial Sequence
  Description of Artificial Sequence Synthetic oligonucleotide
ugaacagcaaguucuauaa

SEQ ID NO: 896 - RNA, 19 nt, Artificial Sequence
  Description of Artificial Sequence Synthetic oligonucleotide
agagcgggacuauaccaaa

SEQ ID NO: 897 - RNA, 19 nt, Artificial Sequence
  Description of Artificial Sequence Synthetic oligonucleotide
ugacucauccuaacuaaca

SEQ ID NO: 898 - RNA, 19 nt, Artificial Sequence
  Description of Artificial Sequence Synthetic oligonucleotide
cugcccagaucgugugcua

SEQ ID NO: 899 - RNA, 19 nt, Artificial Sequence
  Description of Artificial Sequence Synthetic oligonucleotide
accguuaccaaaaagucca

SEQ ID NO: 900 - RNA, 19 nt, Artificial Sequence
  Description of Artificial Sequence Synthetic oligonucleotide
uuaccaaaaaguccugaca

SEQ ID NO: 901 - RNA, 19 nt, Artificial Sequence
  Description of Artificial Sequence Synthetic oligonucleotide
gaccuuagagcacaaccaa

SEQ ID NO: 902 - RNA, 19 nt, Artificial Sequence
  Description of Artificial Sequence Synthetic oligonucleotide
aucacggaguacuguugca

SEQ ID NO: 903 - RNA, 19 nt, Artificial Sequence
  Description of Artificial Sequence Synthetic oligonucleotide
auggcgaccugcucaacua

SEQ ID NO: 904 - RNA, 19 nt, Artificial Sequence
  Description of Artificial Sequence Synthetic oligonucleotide
gcuuccaagaauugcauca

SEQ ID NO: 905 - RNA, 19 nt, Artificial Sequence
  Description of Artificial Sequence Synthetic oligonucleotide
gugucuacacgguucagaa

SEQ ID NO: 906 - RNA, 19 nt, Artificial Sequence
  Description of Artificial Sequence Synthetic oligonucleotide
gguucagagcgacgucuga

SEQ ID NO: 907 - RNA, 19 nt, Artificial Sequence
  Description of Artificial Sequence Synthetic oligonucleotide
uucagagcgacgucuggua

SEQ ID NO: 908 - RNA, 19 nt, Artificial Sequence
  Description of Artificial Sequence Synthetic oligonucleotide
gcaaguucuauaaacugga

SEQ ID NO: 909 - RNA, 19 nt, Artificial Sequence
  Description of Artificial Sequence Synthetic oligonucleotide
acaaacucugccuucggua

SEQ ID NO: 910 - RNA, 19 nt, Artificial Sequence
  Description of Artificial Sequence Synthetic oligonucleotide
ucggucauuucacucaaca

SEQ ID NO: 911 - RNA, 19 nt, Artificial Sequence
  Description of Artificial Sequence Synthetic oligonucleotide
cugacucauccuaacuaaa

SEQ ID NO: 912 - RNA, 19 nt, Artificial Sequence
  Description of Artificial Sequence Synthetic oligonucleotide
ccacauuaaacuaacagca

SEQ ID NO: 913 - RNA, 19 nt, Artificial Sequence
  Description of Artificial Sequence Synthetic oligonucleotide
cagcguugauguuaacuua

SEQ ID NO: 914 - RNA, 19 nt, Artificial Sequence
  Description of Artificial Sequence Synthetic oligonucleotide
cgaucaaguagauuuccaa

SEQ ID NO: 915 - RNA, 19 nt, Artificial Sequence
  Description of Artificial Sequence Synthetic oligonucleotide
ggaagaucaucgagagcua

SEQ ID NO: 916 - RNA, 19 nt, Artificial Sequence
  Description of Artificial Sequence Synthetic oligonucleotide
ggcaacaguuauacuuuca

SEQ ID NO: 917 - RNA, 19 nt, Artificial Sequence
  Description of Artificial Sequence Synthetic oligonucleotide
ggcuagggacaucaugaaa

SEQ ID NO: 918 - RNA, 19 nt, Artificial Sequence
  Description of Artificial Sequence Synthetic oligonucleotide
ggugaaggauggauaccaa

SEQ ID NO: 919 - RNA, 19 nt, Artificial Sequence
  Description of Artificial Sequence Synthetic oligonucleotide
ggaaauggacugacuuuaa

SEQ ID NO: 920 - RNA, 19 nt, Artificial Sequence
  Description of Artificial Sequence Synthetic oligonucleotide
ccaagcugacucauccuaa

SEQ ID NO: 921 - RNA, 19 nt, Artificial Sequence
  Description of Artificial Sequence Synthetic oligonucleotide
cauuaaacuaacagcauua

SEQ ID NO: 922 - DNA, 21 bp, Artificial Sequence
  Description of Artificial Sequence Synthetic oligonucleotide
  Description of Combined DNA/RNA Molecule Synthetic oligonucleotide
tcgaugaucuuccagcggauu

SEQ ID NO: 923 - DNA, 21 bp, Artificial Sequence
  Description of Artificial Sequence Synthetic oligonucleotide
  Description of Combined DNA/RNA Molecule Synthetic oligonucleotide
tcccucauagcucucgauguu

SEQ ID NO: 924 - DNA, 21 bp, Artificial Sequence
  Description of Artificial Sequence Synthetic oligonucleotide
  Description of Combined DNA/RNA Molecule Synthetic oligonucleotide
tuauagaacuugcuguucauu

SEQ ID NO: 925 - DNA, 21 bp, Artificial Sequence
  Description of Artificial Sequence Synthetic oligonucleotide
  Description of Combined DNA/RNA Molecule Synthetic oligonucleotide
tuugguauagucccgcucuuu

SEQ ID NO: 926 - DNA, 21 bp, Artificial Sequence
  Description of Artificial Sequence Synthetic oligonucleotide
  Description of Combined DNA/RNA Molecule Synthetic oligonucleotide
tguuaguuaggaugagucauu

SEQ ID NO: 927 - DNA, 21 bp, Artificial Sequence
  Description of Artificial Sequence Synthetic oligonucleotide
  Description of Combined DNA/RNA Molecule Synthetic oligonucleotide
tcgaugaucuuccagcggauu

SEQ ID NO: 928 - DNA, 21 bp, Artificial Sequence
  Description of Artificial Sequence Synthetic oligonucleotide
  Description of Combined DNA/RNA Molecule Synthetic oligonucleotide
tcccucauagcucucgauguu

SEQ ID NO: 929 - DNA, 21 bp, Artificial Sequence
  Description of Artificial Sequence Synthetic oligonucleotide
  Description of Combined DNA/RNA Molecule Synthetic oligonucleotide
tuauagaacuugcuguucauu

SEQ ID NO: 930 - DNA, 21 bp, Artificial Sequence
  Description of Artificial Sequence Synthetic oligonucleotide
  Description of Combined DNA/RNA Molecule Synthetic oligonucleotide
tuugguauagucccgcucuuu

SEQ ID NO: 931 - DNA, 21 bp, Artificial Sequence
  Description of Artificial Sequence Synthetic oligonucleotide
  Description of Combined DNA/RNA Molecule Synthetic oligonucleotide
tguuaguuaggaugagucauu

SEQ ID NO: 932 - DNA, 21 bp, Artificial Sequence
  Description of Artificial Sequence Synthetic oligonucleotide
  Description of Combined DNA/RNA Molecule Synthetic oligonucleotide
ucgaugaucuuccagcggauu

SEQ ID NO: 933 - DNA, 21 bp, Artificial Sequence
  Description of Artificial Sequence Synthetic oligonucleotide
  Description of Combined DNA/RNA Molecule Synthetic oligonucleotide
ucccucauagcucucgauguu

SEQ ID NO: 934 - DNA, 21 bp, Artificial Sequence
  Description of Artificial Sequence Synthetic oligonucleotide
  Description of Combined DNA/RNA Molecule Synthetic oligonucleotide
uuauagaacuugcuguucauu

SEQ ID NO: 935 - DNA, 21 bp, Artificial Sequence
  Description of Artificial Sequence Synthetic oligonucleotide
  Description of Combined DNA/RNA Molecule Synthetic oligonucleotide
uuugguauagucccgcucuuu

SEQ ID NO: 936 - DNA, 21 bp, Artificial Sequence
  Description of Artificial Sequence Synthetic oligonucleotide
  Description of Combined DNA/RNA Molecule Synthetic oligonucleotide
uguuaguuaggaugagucauu

SEQ ID NO: 937 - DNA, 21 bp, Artificial Sequence
  Description of Artificial Sequence Synthetic oligonucleotide
  Description of Combined DNA/RNA Molecule Synthetic oligonucleotide
tagcacacgaucugggcaguu

SEQ ID NO: 938 - DNA, 21 bp, Artificial Sequence
  Description of Artificial Sequence Synthetic oligonucleotide
  Description of Combined DNA/RNA Molecule Synthetic oligonucleotide
tggacuuuuugguaacgguuu

SEQ ID NO: 939 - DNA, 21 bp, Artificial Sequence
  Description of Artificial Sequence Synthetic oligonucleotide
  Description of Combined DNA/RNA Molecule Synthetic oligonucleotide
tgucaggacuuuuugguaauu

SEQ ID NO: 940 - DNA, 21 bp, Artificial Sequence
  Description of Artificial Sequence Synthetic oligonucleotide
  Description of Combined DNA/RNA Molecule Synthetic oligonucleotide
tugguugugcucuaaggucuu

SEQ ID NO: 941 - DNA, 21 bp, Artificial Sequence
  Description of Artificial Sequence Synthetic oligonucleotide
  Description of Combined DNA/RNA Molecule Synthetic oligonucleotide
tgcaacaguacuccgugauuu

SEQ ID NO: 942 - DNA, 21 bp, Artificial Sequence
  Description of Artificial Sequence Synthetic oligonucleotide
  Description of Combined DNA/RNA Molecule Synthetic oligonucleotide
taguugagcaggucgccauuu

SEQ ID NO: 943 - DNA, 21 bp, Artificial Sequence
  Description of Artificial Sequence Synthetic oligonucleotide
  Description of Combined DNA/RNA Molecule Synthetic oligonucleotide
tgaugcaauucuuggaagcuu

SEQ ID NO: 944 - DNA, 21 bp, Artificial Sequence
  Description of Artificial Sequence Synthetic oligonucleotide
  Description of Combined DNA/RNA Molecule Synthetic oligonucleotide
tucugaaccguguagacacuu

SEQ ID NO: 945 - DNA, 21 bp, Artificial Sequence
  Description of Artificial Sequence Synthetic oligonucleotide
  Description of Combined DNA/RNA Molecule Synthetic oligonucleotide
tcagacgucgcucugaaccuu

SEQ ID NO: 946 - DNA, 21 bp, Artificial Sequence
  Description of Artificial Sequence Synthetic oligonucleotide
  Description of Combined DNA/RNA Molecule Synthetic oligonucleotide
taccagacgucgcucugaauu

SEQ ID NO: 947 - DNA, 21 bp, Artificial Sequence
  Description of Artificial Sequence Synthetic oligonucleotide
  Description of Combined DNA/RNA Molecule Synthetic oligonucleotide
tccaguuuauagaacuugcuu

SEQ ID NO: 948 - DNA, 21 bp, Artificial Sequence
  Description of Artificial Sequence Synthetic oligonucleotide
  Description of Combined DNA/RNA Molecule Synthetic oligonucleotide
taccgaaggcagaguuuguuu

SEQ ID NO: 949 - DNA, 21 bp, Artificial Sequence
  Description of Artificial Sequence Synthetic oligonucleotide
  Description of Combined DNA/RNA Molecule Synthetic oligonucleotide
tguugagugaaaugaccgauu

SEQ ID NO: 950 - DNA, 21 bp, Artificial Sequence
  Description of Artificial Sequence Synthetic oligonucleotide
  Description of Combined DNA/RNA Molecule Synthetic oligonucleotide
tuuaguuaggaugagucaguu

SEQ ID NO: 951 - DNA, 21 bp, Artificial Sequence
  Description of Artificial Sequence Synthetic oligonucleotide
  Description of Combined DNA/RNA Molecule Synthetic oligonucleotide
tgcuguuaguuuaaugugguu

SEQ ID NO: 952 - DNA, 21 bp, Artificial Sequence
  Description of Artificial Sequence Synthetic oligonucleotide
  Description of Combined DNA/RNA Molecule Synthetic oligonucleotide
taaguuaacaucaacgcuguu

SEQ ID NO: 953 - DNA, 21 bp, Artificial Sequence
  Description of Artificial Sequence Synthetic oligonucleotide
  Description of Combined DNA/RNA Molecule Synthetic oligonucleotide
tuggaaaucuacuugaucguu

SEQ ID NO: 954 - DNA, 21 bp, Artificial Sequence
  Description of Artificial Sequence Synthetic oligonucleotide
  Description of Combined DNA/RNA Molecule Synthetic oligonucleotide
tagcucucgaugaucuuccuu

SEQ ID NO: 955 - DNA, 21 bp, Artificial Sequence
  Description of Artificial Sequence Synthetic oligonucleotide
  Description of Combined DNA/RNA Molecule Synthetic oligonucleotide
tgaaaguauaacuguugccuu

SEQ ID NO: 956 - DNA, 21 bp, Artificial Sequence
  Description of Artificial Sequence Synthetic oligonucleotide
  Description of Combined DNA/RNA Molecule Synthetic oligonucleotide
tuucaugaugucccuagccuu

SEQ ID NO: 957 - DNA, 21 bp, Artificial Sequence
  Description of Artificial Sequence Synthetic oligonucleotide
  Description of Combined DNA/RNA Molecule Synthetic oligonucleotide
tugguauccauccuucaccuu

SEQ ID NO: 958 - DNA, 21 bp, Artificial Sequence
  Description of Artificial Sequence Synthetic oligonucleotide
  Description of Combined DNA/RNA Molecule Synthetic oligonucleotide
tuaaagucaguccauuuccuu

SEQ ID NO: 959 - DNA, 21 bp, Artificial Sequence
  Description of Artificial Sequence Synthetic oligonucleotide
  Description of Combined DNA/RNA Molecule Synthetic oligonucleotide
tuaggaugagucagcuugguu

SEQ ID NO: 960 - DNA, 21 bp, Artificial Sequence
  Description of Artificial Sequence Synthetic oligonucleotide
  Description of Combined DNA/RNA Molecule Synthetic oligonucleotide
taaugcuguuaguuuaauguu

SEQ ID NO: 961 - RNA, 19 nt, Artificial Sequence
  Description of Artificial Sequence Synthetic oligonucleotide
acaucucguucucgguuua

SEQ ID NO: 962 - RNA, 19 nt, Artificial Sequence
  Description of Artificial Sequence Synthetic oligonucleotide
ucucguucucgguuuauca

SEQ ID NO: 963 - RNA, 19 nt, Artificial Sequence
  Description of Artificial Sequence Synthetic oligonucleotide
ugcuuuuucuuauuacuca

SEQ ID NO: 964 - RNA, 19 nt, Artificial Sequence
  Description of Artificial Sequence Synthetic oligonucleotide
guaucacaucgguuauuua

SEQ ID NO: 965 - RNA, 19 nt, Artificial Sequence
  Description of Artificial Sequence Synthetic oligonucleotide
uauuuuggcggaaucuuca

SEQ ID NO: 966 - RNA, 19 nt, Artificial Sequence
  Description of Artificial Sequence Synthetic oligonucleotide
acaucucguucucgguuua

SEQ ID NO: 967 - RNA, 19 nt, Artificial Sequence
  Description of Artificial Sequence Synthetic oligonucleotide
ucucguucucgguuuauca

SEQ ID NO: 968 - RNA, 19 nt, Artificial Sequence
  Description of Artificial Sequence Synthetic oligonucleotide
ugcuuuuucuuauuacuca

SEQ ID NO: 969 - RNA, 19 nt, Artificial Sequence
  Description of Artificial Sequence Synthetic oligonucleotide
guaucacaucgguuauuua

SEQ ID NO: 970 - RNA, 19 nt, Artificial Sequence
  Description of Artificial Sequence Synthetic oligonucleotide
uauuuuggcggaaucuuca

SEQ ID NO: 971 - RNA, 19 nt, Artificial Sequence
  Description of Artificial Sequence Synthetic oligonucleotide
acaucucguucucgguuua

SEQ ID NO: 972 - RNA, 19 nt, Artificial Sequence
  Description of Artificial Sequence Synthetic oligonucleotide
ucucguucucgguuuauca

SEQ ID NO: 973 - RNA, 19 nt, Artificial Sequence
  Description of Artificial Sequence Synthetic oligonucleotide
ugcuuuuucuuauuacuca

SEQ ID NO: 974 - RNA, 19 nt, Artificial Sequence
  Description of Artificial Sequence Synthetic oligonucleotide
guaucacaucgguuauuua

SEQ ID NO: 975 - RNA, 19 nt, Artificial Sequence
  Description of Artificial Sequence Synthetic oligonucleotide
uauuuuggcggaaucuuca

SEQ ID NO: 976 - RNA, 19 nt, Artificial Sequence
  Description of Artificial Sequence Synthetic oligonucleotide
cuguccacaucucguucua

SEQ ID NO: 977 - RNA, 19 nt, Artificial Sequence
  Description of Artificial Sequence Synthetic oligonucleotide
uccacaucucguucucgga

SEQ ID NO: 978 - RNA, 19 nt, Artificial Sequence
  Description of Artificial Sequence Synthetic oligonucleotide
cggugaagaagucaccaca

SEQ ID NO: 979 - RNA, 19 nt, Artificial Sequence
  Description of Artificial Sequence Synthetic oligonucleotide
ucaccaccuuuuuugauua

SEQ ID NO: 980 - RNA, 19 nt, Artificial Sequence
  Description of Artificial Sequence Synthetic oligonucleotide
ugacgugaagcaaauugga

SEQ ID NO: 981 - RNA, 19 nt, Artificial Sequence
  Description of Artificial Sequence Synthetic oligonucleotide
ugcaaaaagcugaagugca

SEQ ID NO: 982 - RNA, 19 nt, Artificial Sequence
  Description of Artificial Sequence Synthetic oligonucleotide
aaagcugaagugcuugaca

SEQ ID NO: 983 - RNA, 19 nt, Artificial Sequence
  Description of Artificial Sequence Synthetic oligonucleotide
uugggaaugcaaugugcaa

SEQ ID NO: 984 - RNA, 19 nt, Artificial Sequence
  Description of Artificial Sequence Synthetic oligonucleotide
agggcuguaucacaucgga

SEQ ID NO: 985 - RNA, 19 nt, Artificial Sequence
  Description of Artificial Sequence Synthetic oligonucleotide
ggcuguaucacaucgguua

SEQ ID NO: 986 - RNA, 19 nt, Artificial Sequence
  Description of Artificial Sequence Synthetic oligonucleotide
cuguaucacaucgguuaua

SEQ ID NO: 987 - RNA, 19 nt, Artificial Sequence
  Description of Artificial Sequence Synthetic oligonucleotide
caucgguuauuuuggcgga

SEQ ID NO: 988 - RNA, 19 nt, Artificial Sequence
  Description of Artificial Sequence Synthetic oligonucleotide
ucaucuuuacuaaaugcca

SEQ ID NO: 989 - RNA, 19 nt, Artificial Sequence
  Description of Artificial Sequence Synthetic oligonucleotide
gcccuuauuuuccacgaga

SEQ ID NO: 990 - RNA, 19 nt, Artificial Sequence
  Description of Artificial Sequence Synthetic oligonucleotide
uccacgaggauggaauaaa

SEQ ID NO: 991 - RNA, 19 nt, Artificial Sequence
  Description of Artificial Sequence Synthetic oligonucleotide
gguuuaucagaaauaccaa

SEQ ID NO: 992 - RNA, 19 nt, Artificial Sequence
  Description of Artificial Sequence Synthetic oligonucleotide
caccaccuuuuuugauuaa

SEQ ID NO: 993 - RNA, 19 nt, Artificial Sequence
  Description of Artificial Sequence Synthetic oligonucleotide
ccaccuuuuuugauuauga

SEQ ID NO: 994 - RNA, 19 nt, Artificial Sequence
  Description of Artificial Sequence Synthetic oligonucleotide
cggugcucccugucauaaa

SEQ ID NO: 995 - RNA, 19 nt, Artificial Sequence
  Description of Artificial Sequence Synthetic oligonucleotide
cucccugucauaaauuuga

SEQ ID NO: 996 - RNA, 19 nt, Artificial Sequence
  Description of Artificial Sequence Synthetic oligonucleotide
gucauaaauuugacgugaa

SEQ ID NO: 997 - RNA, 19 nt, Artificial Sequence
  Description of Artificial Sequence Synthetic oligonucleotide
caggaaucaucuuuacuaa

SEQ ID NO: 998 - RNA, 19 nt, Artificial Sequence
  Description of Artificial Sequence Synthetic oligonucleotide
aggaaucaucuuuacuaaa

SEQ ID NO: 999 - RNA, 19 nt, Artificial Sequence
  Description of Artificial Sequence Synthetic oligonucleotide
cacgaggauggaauaauua

SEQ ID NO: 1000 - RNA, 19 nt, Artificial Sequence
  Description of Artificial Sequence Synthetic oligonucleotide
gaauaauuuccacacaaua

SEQ ID NO: 1001 - RNA, 19 nt, Artificial Sequence
  Description of Artificial Sequence Synthetic oligonucleotide
cacacaauaaugaggaaca

SEQ ID NO: 1002 - DNA, 21 bp, Artificial Sequence
  Description of Artificial Sequence Synthetic oligonucleotide
  Description of Combined DNA/RNA Molecule Synthetic oligonucleotide
taaaccgagaacgagauguuu

SEQ ID NO: 1003 - DNA, 21 bp, Artificial Sequence
  Description of Artificial Sequence Synthetic oligonucleotide
  Description of Combined DNA/RNA Molecule Synthetic oligonucleotide
tgauaaaccgagaacgagauu

SEQ ID NO: 1004 - DNA, 21 bp, Artificial Sequence
  Description of Artificial Sequence Synthetic oligonucleotide
  Description of Combined DNA/RNA Molecule Synthetic oligonucleotide
tgaguaauaagaaaaagcauu

SEQ ID NO: 1005 - DNA, 21 bp, Artificial Sequence
  Description of Artificial Sequence Synthetic oligonucleotide
  Description of Combined DNA/RNA Molecule Synthetic oligonucleotide
taaauaaccgaugugauacuu

SEQ ID NO: 1006 - DNA, 21 bp, Artificial Sequence
  Description of Artificial Sequence Synthetic oligonucleotide
  Description of Combined DNA/RNA Molecule Synthetic oligonucleotide
tgaagauuccgccaaaauauu

SEQ ID NO: 1007 - DNA, 21 bp, Artificial Sequence
  Description of Artificial Sequence Synthetic oligonucleotide
  Description of Combined DNA/RNA Molecule Synthetic oligonucleotide
taaaccgagaacgagauguuu

SEQ ID NO: 1008 - DNA, 21 bp, Artificial Sequence
  Description of Artificial Sequence Synthetic oligonucleotide
  Description of Combined DNA/RNA Molecule Synthetic oligonucleotide
tgauaaaccgagaacgagauu

SEQ ID NO: 1009 - DNA, 21 bp, Artificial Sequence
  Description of Artificial Sequence Synthetic oligonucleotide
  Description of Combined DNA/RNA Molecule Synthetic oligonucleotide
tgaguaauaagaaaaagcauu

SEQ ID NO: 1010 - DNA, 21 bp, Artificial Sequence
  Description of Artificial Sequence Synthetic oligonucleotide
  Description of Combined DNA/RNA Molecule Synthetic oligonucleotide
taaauaaccgaugugauacuu

SEQ ID NO: 1011 - DNA, 21 bp, Artificial Sequence
  Description of Artificial Sequence Synthetic oligonucleotide
  Description of Combined DNA/RNA Molecule Synthetic oligonucleotide
tgaagauuccgccaaaauauu

SEQ ID NO: 1012 - DNA, 21 bp, Artificial Sequence
  Description of Artificial Sequence Synthetic oligonucleotide
  Description of Combined DNA/RNA Molecule Synthetic oligonucleotide
uaaaccgagaacgagauguuu

SEQ ID NO: 1013 - DNA, 21 bp, Artificial Sequence
  Description of Artificial Sequence Synthetic oligonucleotide
  Description of Combined DNA/RNA Molecule Synthetic oligonucleotide
ugauaaaccgagaacgagauu

SEQ ID NO: 1014 - DNA, 21 bp, Artificial Sequence
  Description of Artificial Sequence Synthetic oligonucleotide
  Description of Combined DNA/RNA Molecule Synthetic oligonucleotide
ugaguaauaagaaaaagcauu

SEQ ID NO: 1015 - DNA, 21 bp, Artificial Sequence
  Description of Artificial Sequence Synthetic oligonucleotide
  Description of Combined DNA/RNA Molecule Synthetic oligonucleotide
uaaauaaccgaugugauacuu

SEQ ID NO: 1016 - DNA, 21 bp, Artificial Sequence
  Description of Artificial Sequence Synthetic oligonucleotide
  Description of Combined DNA/RNA Molecule Synthetic oligonucleotide
ugaagauuccgccaaaauauu

SEQ ID NO: 1017 - DNA, 21 bp, Artificial Sequence
  Description of Artificial Sequence Synthetic oligonucleotide
  Description of Combined DNA/RNA Molecule Synthetic oligonucleotide
tagaacgagauguggacaguu

SEQ ID NO: 1018 - DNA, 21 bp, Artificial Sequence
  Description of Artificial Sequence Synthetic oligonucleotide
  Description of Combined DNA/RNA Molecule Synthetic oligonucleotide
tccgagaacgagauguggauu

SEQ ID NO: 1019 - DNA, 21 bp, Artificial Sequence
  Description of Artificial Sequence Synthetic oligonucleotide
  Description of Combined DNA/RNA Molecule Synthetic oligonucleotide
tguggugacuucuucaccguu

SEQ ID NO: 1020 - DNA, 21 bp, Artificial Sequence
  Description of Artificial Sequence Synthetic oligonucleotide
  Description of Combined DNA/RNA Molecule Synthetic oligonucleotide
taaucaaaaaagguggugauu

SEQ ID NO: 1021 - DNA, 21 bp, Artificial Sequence
  Description of Artificial Sequence Synthetic oligonucleotide
  Description of Combined DNA/RNA Molecule Synthetic oligonucleotide
tccaauuugcuucacgucauu

SEQ ID NO: 1022 - DNA, 21 bp, Artificial Sequence
  Description of Artificial Sequence Synthetic oligonucleotide
  Description of Combined DNA/RNA Molecule Synthetic oligonucleotide
tgcacuucagcuuuuugcauu

SEQ ID NO: 1023 - DNA, 21 bp, Artificial Sequence
  Description of Artificial Sequence Synthetic oligonucleotide
  Description of Combined DNA/RNA Molecule Synthetic oligonucleotide
tgucaagcacuucagcuuuuu

SEQ ID NO: 1024 - DNA, 21 bp, Artificial Sequence
  Description of Artificial Sequence Synthetic oligonucleotide
  Description of Combined DNA/RNA Molecule Synthetic oligonucleotide
tugcacauugcauucccaauu

SEQ ID NO: 1025 - DNA, 21 bp, Artificial Sequence
  Description of Artificial Sequence Synthetic oligonucleotide
  Description of Combined DNA/RNA Molecule Synthetic oligonucleotide
tccgaugugauacagcccuuu

SEQ ID NO: 1026 - DNA, 21 bp, Artificial Sequence
  Description of Artificial Sequence Synthetic oligonucleotide
  Description of Combined DNA/RNA Molecule Synthetic oligonucleotide
taaccgaugugauacagccuu

SEQ ID NO: 1027 - DNA, 21 bp, Artificial Sequence
  Description of Artificial Sequence Synthetic oligonucleotide
  Description of Combined DNA/RNA Molecule Synthetic oligonucleotide
tauaaccgaugugauacaguu

SEQ ID NO: 1028 - DNA, 21 bp, Artificial Sequence
  Description of Artificial Sequence Synthetic oligonucleotide
  Description of Combined DNA/RNA Molecule Synthetic oligonucleotide
tccgccaaaauaaccgauguu

SEQ ID NO: 1029 - DNA, 21 bp, Artificial Sequence
  Description of Artificial Sequence Synthetic oligonucleotide
  Description of Combined DNA/RNA Molecule Synthetic oligonucleotide
tggcauuuaguaaagaugauu

SEQ ID NO: 1030 - DNA, 21 bp, Artificial Sequence
  Description of Artificial Sequence Synthetic oligonucleotide
  Description of Combined DNA/RNA Molecule Synthetic oligonucleotide
tcucguggaaaauaagggcuu

SEQ ID NO: 1031 - DNA, 21 bp, Artificial Sequence
  Description of Artificial Sequence Synthetic oligonucleotide
  Description of Combined DNA/RNA Molecule Synthetic oligonucleotide
tuuauuccauccucguggauu

SEQ ID NO: 1032 - DNA, 21 bp, Artificial Sequence
  Description of Artificial Sequence Synthetic oligonucleotide
  Description of Combined DNA/RNA Molecule Synthetic oligonucleotide
tugguauuucugauaaaccuu

SEQ ID NO: 1033 - DNA, 21 bp, Artificial Sequence
  Description of Artificial Sequence Synthetic oligonucleotide
  Description of Combined DNA/RNA Molecule Synthetic oligonucleotide
tuaaucaaaaaaggugguguu

SEQ ID NO: 1034 - DNA, 21 bp, Artificial Sequence
  Description of Artificial Sequence Synthetic oligonucleotide
  Description of Combined DNA/RNA Molecule Synthetic oligonucleotide
tcauaaucaaaaaaggugguu

SEQ ID NO: 1035 - DNA, 21 bp, Artificial Sequence
  Description of Artificial Sequence Synthetic oligonucleotide
  Description of Combined DNA/RNA Molecule Synthetic oligonucleotide
tuuaugacagggagcaccguu

SEQ ID NO: 1036 - DNA, 21 bp, Artificial Sequence
  Description of Artificial Sequence Synthetic oligonucleotide
  Description of Combined DNA/RNA Molecule Synthetic oligonucleotide
tcaaauuuaugacagggaguu

SEQ ID NO: 1037 - DNA, 21 bp, Artificial Sequence
  Description of Artificial Sequence Synthetic oligonucleotide
  Description of Combined DNA/RNA Molecule Synthetic oligonucleotide
tucacgucaaauuuaugacuu

SEQ ID NO: 1038 - DNA, 21 bp, Artificial Sequence
  Description of Artificial Sequence Synthetic oligonucleotide
  Description of Combined DNA/RNA Molecule Synthetic oligonucleotide
tuaguaaagaugauuccuguu

SEQ ID NO: 1039 - DNA, 21 bp, Artificial Sequence
  Description of Artificial Sequence Synthetic oligonucleotide
  Description of Combined DNA/RNA Molecule Synthetic oligonucleotide
tuuaguaaagaugauuccuuu

SEQ ID NO: 1040 - DNA, 21 bp, Artificial Sequence
  Description of Artificial Sequence Synthetic oligonucleotide
  Description of Combined DNA/RNA Molecule Synthetic oligonucleotide
taauuauuccauccucguguu

SEQ ID NO: 1041 - DNA, 21 bp, Artificial Sequence
  Description of Artificial Sequence Synthetic oligonucleotide
  Description of Combined DNA/RNA Molecule Synthetic oligonucleotide
tauuguguggaaauuauucuu

SEQ ID NO: 1042 - DNA, 21 bp, Artificial Sequence
  Description of Artificial Sequence Synthetic oligonucleotide
  Description of Combined DNA/RNA Molecule Synthetic oligonucleotide
tguuccucauuauuguguguu

SEQ ID NO: 1043 - DNA, 21 bp, Artificial Sequence
  Description of Artificial Sequence Synthetic oligonucleotide
  Description of Combined DNA/RNA Molecule Synthetic oligonucleotide
cuacucaacuuucuccgaatt

SEQ ID NO: 1044 - DNA, 21 bp, Artificial Sequence
  Description of Artificial Sequence Synthetic oligonucleotide
  Description of Combined DNA/RNA Molecule Synthetic oligonucleotide
ugcuaaacgucucugcaaatt

SEQ ID NO: 1045 - DNA, 21 bp, Artificial Sequence
  Description of Artificial Sequence Synthetic oligonucleotide
  Description of Combined DNA/RNA Molecule Synthetic oligonucleotide
cuuacgcugaguacuucgatt

SEQ ID NO: 1046 - DNA, 21 bp, Artificial Sequence
  Description of Artificial Sequence Synthetic oligonucleotide
  Description of Combined DNA/RNA Molecule Synthetic oligonucleotide
uucggagaaaguugaguagtt

SEQ ID NO: 1047 - DNA, 21 bp, Artificial Sequence
  Description of Artificial Sequence Synthetic oligonucleotide
  Description of Combined DNA/RNA Molecule Synthetic oligonucleotide
uuugcagagacguuuagcatt

SEQ ID NO: 1048 - DNA, 21 bp, Artificial Sequence
  Description of Artificial Sequence Synthetic oligonucleotide
  Description of Combined DNA/RNA Molecule Synthetic oligonucleotide
ucgaaguacucagcguaagtt

SEQ ID NO: 1049 - DNA, 21 bp, Artificial Sequence
  Description of Artificial Sequence Synthetic oligonucleotide
  Description of Combined DNA/RNA Molecule Synthetic oligonucleotide
ggaugaaguggagauuagutt

SEQ ID NO: 1050 - DNA, 21 bp, Artificial Sequence
  Description of Artificial Sequence Synthetic oligonucleotide
  Description of Combined DNA/RNA Molecule Synthetic oligonucleotide
acuaaucuccacuucaucctt